SEQUENCE LISTING (5 sequences)

SEQ ID NO: 1 - protein, 519 aa, Artificial Sequence
  Modified Sequence
GKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTGSSELYRQSLEIISRYLREQATGAADTAPMGASGATSRKALETLRRVGDGVQRNHETAFQGMLRKLDIKNEDDVKSLSRVMIHVFSDGVTNWGRIVTLISFGAFVAKHLKTINQESCIEPLAESITDVLVRTKRDWLVKQRGWDGFVEFFHV

SEQ ID NO: 2 - protein, 26 aa, Artificial Sequence
  MOD_RES 1: Biotin-PEG-PEG-PEG-PEG
  Modified Sequence
  MOD_RES 8: Norvalin
  MOD_RES 26: AMIDATION
PAELEVEVATQLRRFGDKLNFRQKLL

SEQ ID NO: 3 - protein, 212 aa, Artificial Sequence
  Modified Sequence
  MOD_RES 212: HHHHHH
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNNAAAESRKGQERFNR

SEQ ID NO: 4 - protein, 25 aa, Artificial Sequence
  MOD_RES 1: Biotin-PEG-PEG-PEG-PEG
  Modofied Sequence
  MOD_RES 15: Norleucin
  MOD_RES 25: AMIDATION
NLWAAQRYGRELRRLSDEFVDSFKK

SEQ ID NO: 5 - protein, 211 aa, Artificial Sequence
  Modfied Sequence
  MOD_RES 211: HHHHHH
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFD

(19) United States
(12) Patent Application Publication
(10) Pub. No.: US 2021/0253598 A1
(43) Pub. Date: Aug. 19, 2021

(54) ARYL ANNULATED MACROCYCLIC INDOLE DERIVATIVES

(71) Applicants: Bayer Aktiengesellschaft, Leverkusen (DE); Bayer Pharma Aktiengesellschaft, Berlin (DE); The Broad Institute, Inc., Cambridge, MA (US)

(72) Inventors: Kai Thede, Leverkusen (DE); Anne Mengel, Leverkusen (DE); Clara Christ, Leverkusen (DE); Joachim Kuhnke, Leverkusen (DE); Sarah Anna Liesa Johannes, Leverkusen (DE); Philipp Buchgraber, Leverkusen (DE); Ulrich Klar, Leverkusen (DE); Ulrike Sack, Leverkusen (DE); Stefan Kaulfuss, Leverkusen (DE); Amaury Ernesto Fernandez-Montalvan, Leverkusen (DE); Nicolas Werbeck, Leverkusen (DE); Ursula Mönning, Leverkusen (DE); Steven James Ferrara, Cambridge, MA (US); Michael H. Serrano-Wu, Cambridge, MA (US); Christopher Lemke, Cambridge, MA (US); David McKinney, Cambridge, MA (US); Mark Fitzgerald, Cambridge, MA (US); Christopher Nasveschuk, Cambridge, MA (US); Kiel Lazarski, Cambridge, MA (US); Laura Furst, Cambridge, MA (US); Guo Wei, Cambridge, MA (US); Patrick Ryan MacCarren, Cambridge, MA (US); Rebecca Ann Harvey, Lancashire (GB); Craig Wilson, Buxton (GB)

(21) Appl. No.: 16/764,560

(22) PCT Filed: Nov. 15, 2018
(86) PCT No.: PCT/EP2018/081374

§ 371 Date: May 15, 2020

Related U.S. Application Data
(60) Provisional application No. 62/587,689, filed on Nov. 17, 2017.

(51) Int. Cl. C07D 498/14; A61K 45/06; C07D 498/22; A61K 31/437; A61K 31/4162; A61K 31/4985; C07D 487/14; A61K 31/424; A61P 35/00; A61P 35/02

(52) U.S. Cl. CPC C07D 498/14; A61K 45/06; C07D 498/22; A61K 31/437; A61P 35/02; A61K 31/4985; C07D 487/14; A61K 31/424; A61P 35/00; A61K 31/4162

(57) ABSTRACT

The present invention relates to aryl annulated macrocyclic indole derivatives of general formula (I):

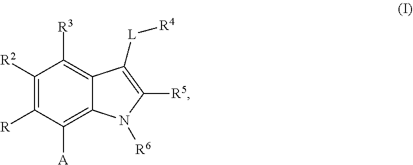

in which R¹, R², R³, R⁴, R⁵, R⁶, A and L are as defined herein, methods of preparing said compounds, intermediate compounds useful for preparing said compounds, pharmaceutical compositions and combinations comprising said compounds, and the use of said compounds for manufacturing pharmaceutical compositions for the treatment or prophylaxis of diseases, in particular of hyperproliferative disorders, as a sole agent or in combination with other active ingredients.

### BACKGROUND

The present invention covers macrocyclic indole derivatives of general formula (I) which inhibit the antiapoptotic activity of MCL-1 by inhibiting its interaction with proapoptotic proteins.

Apoptosis, also called programmed cell death, is a natural process which allows a damaged or unwanted cell to die in a controlled manner. Deregulation of this process leads to unrestrained cell proliferation and is thus a hallmark of cancer (Hanahan and Weinberg, 2011).

Apoptosis is highly controlled by proteins of the B-cell lymphoma 2 (BCL-2) family. These proteins are characterized by their conserved regions known as BCL-2 homology (BH) domains (BH1-BH4) (Korsmeyer, 1999) through which they interact with each other. The BCL-2 family can be divided into pro-apoptotic members including BAX, BAK, BAD, BID, BIM, BMF, NOXA, and PUMA, which induce cell death, and anti-apoptotic members such as BCL-2, BCL-XL, BCL-w, Bfl1-AI, and myeloid cell leukemia-1 (MCL-1) which block apoptosis (Adams and Cory, 2007). The relative expression level of these two opponent groups of the BCL-2 family will decide if a cell will go into apoptosis or not.

MCL-1 has been identified as an important therapeutic target in cancer. MCL-1 is highly expressed in a variety of human cancers, and amplification of the MCL-1 locus is one of the most frequent somatic genetic events in human cancer, further pointing to its centrality in the pathogenesis of malignancy (Beroukhim et al., 2010). Its expression has been linked to deregulated anti-apoptotic pathways in cancer, thus leading to increased cancer cell survival, tumor development (Zhou et al., 2001) and resistance to anticancer therapies (Wertz et al., 2011). MCL-1 protein has been shown to mediate survival in models of acute myeloid leukemia (Glaser et al., 2012), lymphomas (Kelly et al., 2014) and multiple myeloma (Zhang et al., 2002). Many chemotherapeutics as well as radiation aim at inducing apoptosis in cancer cells. However, in malignant cells, apoptotic signaling is often deregulated, leading to uncontrolled growth and therapeutic resistance. One key resistance mechanism to apoptosis is to upregulate or genetically amplify MCL-1.

MCI-1 is a major inhibitor of apoptosis in cancer. MCL-1 is the largest member of the anti-apoptotic BCI-2 proteins. Its expression is tightly controlled with a half-life of only 1-4 h. With its BH-3 domain, MCL-1 tightly binds to BH-3 only containing pro-apoptotic proteins such as BAK or BAX and hinders them from inducing pores in the mitochondrial membrane, thereby blocking the intrinsic apoptotic pathway.

Thus, the specific inhibition of the interaction of MCL-1 with BH-3 only containing pro-apoptotic proteins like BAK or BAX represents a very attractive therapeutic principle to induce apoptosis in cancer cells and to address resistance against chemotherapeutics, radiation and new targeted agents. However, from WO 2015/148854, US 2016/0106731, WO 2008/130970, some indole derivatives are known as MCL-1 inhibitors. As no inhibitors have shown effectivity in the clinic yet, there is still a need for further MCL-1 inhibitors to be provided.

### SUMMARY

It has now been found that the compounds of the present invention effectively inhibit the activity of the anti-apoptotic BCL-2 family member Myeloid cell leukemia-1 (MCL-1) protein for which data are given in the biological experimental section and may therefore be used for the treatment or prophylaxis of hyperproliferative disorders, such as cancer disorders.

In accordance with a first aspect, the present invention provides compounds of general formula (I):

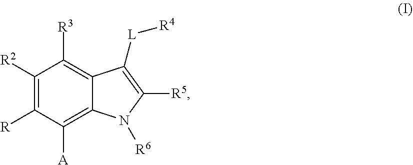

wherein
A is

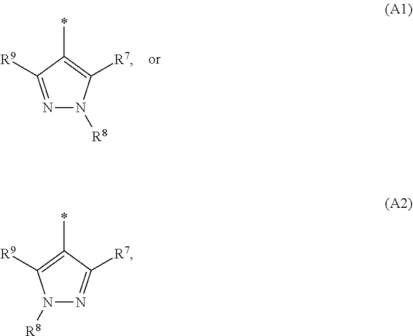

wherein R⁶ and R⁷, together with two carbon atoms of the pyrazole ring, two carbon atoms of the indole moiety and the nitrogen atom to which R⁶ is attached, form a 9- to 16-membered ring and * is the point of attachment of these moieties to the indole carbon atom bearing the A substituent
or
A is

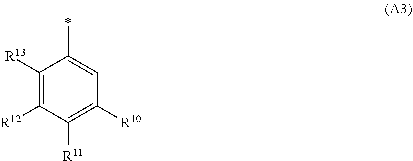

wherein optionally one or two of the groups selected from CR¹¹, CR¹² and CR¹³ are replaced by a nitrogen atom,
wherein R⁶ and R¹⁰, together with three carbon atoms of the phenyl ring, two carbon atoms of the indole moiety and the nitrogen atom to which R⁶ is attached, form a 9- to 16-membered ring and * is the point of attachment of these moieties to the indole carbon atom bearing the A substituent;
R¹ and R² are each independently selected from a hydrogen atom, a halogen atom, a cyano group, a C₁-C₃-alkyl group, and a C₁-C₃-alkoxy group;
R³ is selected from a hydrogen atom, a halogen atom, a cyano group, a C₁-C₃-alkyl group, a C₁-C₃-haloalkyl group, a C₁-C₃-alkoxy group, a C₁-C₃-alkylthio group, a —S(O)—(C₁-C₃-alkyl) group, a —S(O)₂—(C₁-C₃-alkyl) group, a C₁-C₃-haloalkoxy group, a C₁-C₃-haloalkylthio group, and a C₃-C₅-cycloalkyl group;
R⁴ is selected from an aryl group and a heteroaryl group, each of which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group, a C₁-C₃-thioalkyl group, a C₁-C₃-haloalkoxy group, a (C₁-C₃)-haloalkyl-S— group, and a C₃-C₅-cycloalkyl group;
L is a group —(CH₂)ₘ-E- wherein any CH₂ group is unsubstituted or substituted with one or two substituents and each substituent is independently selected from a halogen atom, a cyano group, a hydroxyl group, a C₁-C₃-alkyl group, and a C₁-C₃-alkoxy group, or two substituents are optionally taken together with their intervening atoms to form a saturated or partially unsaturated 3-6-membered cycloalkyl ring, or a 3-8 membered saturated or partially unsaturated heterocyclic ring having 1-2 heteroatoms independently selected from an oxygen atom, a sulfur atom, a —S(O)— group, a —S(O)₂— group, and a —NR¹⁴— group;
E is a bond, an oxygen atom, a sulfur atom, a —S(O)— group, a —S(O)₂— group or a —NR¹⁴— group and constitutes the connecting element to R⁴,
m is 2, 3, or 4;
R⁵ is selected from a COOH group, a

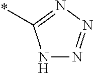 [SMILES: *c1nnn[nH]1]

group, a —C(O)—NHS(O)₂(C₁-C₆-alkyl) group, a —C(O)—NHS(O)₂(C₃-C₆-cycloalkyl) group, a —C(O)—NHS(O)₂(aryl) group, a —C(O)—NHS(O)₂(CH₂)ₛNHCO(C₁-C₆-alkyl) group, a —C(O)—NHS(O)₂(CH₂)ₛNHCO(C₃-C₆-cycloalkyl) group, and a —C(O)—NHS(O)₂(CH₂)ₛNHCO(aryl) group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, a C₁-C₃-alkoxy group, a C₁-C₃-haloalkoxy group, and a (heterocycloalkyl)-(C₁-C₃-alkylene)- group, and where a —CH═CH— group in any alkenylene group can be replaced by a 1,2-cyclopropylene group, and said 1,2-cyclopropylene group is unsubstituted or substituted one or two times with a halogen atom or a C₁-C₂-alkyl group, wherein ^{#} is the point of attachment with the indole nitrogen atom and ^{##} is the point of attachment with the pyrazole carbon atom bearing the R⁷ substituent;
—R⁶-R¹⁰— is ^{#}—(CH₂)ₙ-(G)-(CH₂)ᵣ—(B)ₜ—(CH₂)ₚ—^{##}, where one or more CH₂ groups are unsubstituted or substituted with one or more substituents selected from a halogen atom, a hydroxyl group, a NR¹⁷R¹⁸ group, a C₁-C₃-alkyl group, a C₁-C₃-haloalkyl group, a C₁-C₃-alkoxy group, a C₁-C₃-haloalkoxy group and a (heterocycloalkyl)-(C₁-C₃-alkylene)- group, wherein ^{#} is the point of attachment with the indole nitrogen atom and ^{##} is the point of attachment with the carbon atom of the phenyl moiety bearing the R¹⁰ substituent;
n is 1, 2, 3, 4, 5, 6, 7, 8, 9, or 10;
t is 0 or 1;
r is 0, 1, 2, or 3;
s is 0, 1, 2, or 3;
p is 0, 1, 2, 3, 4, 5, or 6;
where the integers selected for variables n, t, r, s, and p result in forming a 9- to 16-membered ring independently from the selection of variable A1, A2 or A3;
B is independently selected from a —C(O)NR¹⁵— group, a —NR¹⁵C(O)— group, a —N(R¹⁵)— group, a —N(R¹⁵)—C(═O)—N(R¹⁵)— group, a —O—C(═O)—N(R¹⁵)— group, a —N(R¹⁵)—C(═O)—O— group, —O—, —S—, —S(O)—, —S(O)₂—, a —S(O)NR¹⁵— group, a —NR¹⁵S(O)— group, a —S(O)₂NR¹⁵— group, a —NR¹⁵S(O)₂— group, a

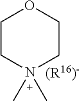

group and a —[N⁺(R²¹R²²)—(R¹⁶)⁻]— group,
G is a 1,2-arylene group or
   a mono- or bicyclic heteroarylene group wherein two vicinal carbon atoms thereof are each bound to one of the adjacent alkylene groups,
   which are unsubstituted or substituted with one or more substituents independently selected from a halogen atom, a cyano group, a C₁-C₃-alkyl group, and a C₁-C₃-alkoxy group;
R⁸ is selected from a hydrogen atom,
   a C₁-C₆-alkyl group, which is unsubstituted or substituted with one or more substituents independently selected from
      a halogen atom, a hydroxyl group, a C₁-C₃-alkoxy group, a C₁-C₃-haloalkoxy group, a C₃-C₆-cycloalkyl group, a heterocycloalkyl group, and a NR²¹R²² group, or
   a C₁-C₃-haloalkyl group, a C₃-C₆-cycloalkyl group, or a C₁-C₆-alkyl group in which one or two not directly adjacent carbon atoms are independently replaced by a heteroatom selected from —O— and —NH—;
R⁹ is selected from a hydrogen atom,
   a C₁-C₄-alkyl group,
   a C₁-C₃-hydroxyalkyl group,
   a C₁-C₄-haloalkyl group,
   a C₁-C₄-haloalkyl-NH—C(O)—O—(C₁-C₃-alkylene)- group,
   a C₂-C₆-haloalkenyl group,
   a C₁-C₆-alkyl-O— group,
   a C₁-C₄-haloalkoxy group,
   a C₁-C₆-alkyl-O—(C₁-C₃-alkylene)- group,
   a (C₃-C₇)-cycloalkyl group,
   a (C₃-C₇)-cycloalkyl-O—(C₁-C₃-alkylene)- group,
   a phenyl-O—(C₁-C₃-alkylene)- group,
   a phenyl-(C₁-C₃-alkylene)-O—(C₁-C₃-alkylene)- group,
   a R¹⁹-(phenylene)-(C₁-C₃-alkylene)-O—(C₁-C₃-alkylene)- group,
   a R¹⁹-(phenylene)-O—(C₁-C₃-alkylene)- group,
   a R¹⁹-(phenylene)-(heteroarylene)-O—(C₁-C₃-alkylene) group,
   a (R¹⁹)-(heterocycloalkylene)-(C₁-C₃-alkylene)- group,
   a (R¹⁹)-(heterocycloalkylene)-(phenylene)-O—(C₁-C₃-alkylene)- group,
   a (heterocycloalkenyl)-(phenylene)-O—(C₁-C₃-alkylene)- group,
   a (R¹⁹)-(heteroarylene)-(C₁-C₃-alkylene)-O—(C₁-C₃-alkylene)- group,
   a (R¹⁹)-(heteroarylene)-(phenylene)-O—(C₁-C₃-alkylene)- group,
   a (R²⁰)—S(O)₂-(phenylene)-O—(C₁-C₃-alkylene)- group,
   a (R²⁰)—S(O)₂—NH-(phenylene)-O—(C₁-C₃-alkylene)- group,
   a (R²⁰)—S(O)₂—NH-(phenylene)-O—(C₁-C₃-alkylene)- group,
   a (R²⁰)—S(O)₂—N(C₁-C₆-alkyl)-(phenylene)-O—(C₁-C₃-alkylene)- group,
   a (R²⁰)—S(O)₂-(heterocycloalkylene)-(phenylene)-O—(C₁-C₃-alkylene)- group,
   a (R¹⁹)-(heterocycloalkylene)-(heteroarylene)-O—(C₁-C₃-alkylene)- group,
   a (R²⁰)-(heteroarylene)-O—(C₁-C₃-alkylene)- group,
   a (R¹⁹)-(heteroarylene)-O—(C₁-C₃-alkylene)- group,
   a (R²⁰)—S(O)₂-(heteroarylene)-O—(C₁-C₃-alkylene)- group,
   a (R²⁰)—S(O)₂-(heterocycloalkylene)-(heteroarylene)-O—(C₁-C₃-alkylene)- group,
   a NR²¹R²²—(C₁-C₃-alkylene)- group,
   a (C₁-C₃-alkyl)-NH—(C₁-C₃-alkylene)- group,
   a (C₁-C₃-haloalkyl)-(C₁-C₃-alkylene)-NH—(C₁-C₃-alkylene)- group,
   a (C₁-C₃-haloalkyl)-NH—(C₁-C₃-alkylene)- group,
   a (C₁-C₃-alkyl)-NH—C(O)—(C₁-C₃-alkylene)- group,
   a (C₁-C₃-alkyl)-NR¹⁵—C(O)—(C₁-C₃-alkylene)- group,
   a (C₁-C₃-alkyl)-C(O)—NH—(C₁-C₃-alkylene)- group,
   a (C₁-C₃-alkyl)-C(O)—NR¹⁵—(C₁-C₃-alkylene)- group,
   a

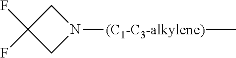

group, and a

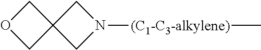

group, where the phenyl ring is unsubstituted or substituted with a halogen atom, a hydroxyl group, or a C₁-C₃-alkoxy group and
the heterocycloalkyl group is unsubstituted or substituted with an oxo (═O) group or is unsubstituted or substituted with one or more substituents independently selected from a halogen atom, a hydroxyl group, a C₁-C₃-alkyl group, and a C₁-C₃-alkoxy group,
or R⁸ and R⁹ together form a 5- or 6-membered ring optionally comprising one or two heteroatoms independently selected from —O— and —NR¹⁴—;
R¹¹ and R¹³ are each independently selected from a hydrogen atom, a halogen atom, a C₁-C₃-alkyl group, and a C₁-C₃-alkoxy group;
R¹² is selected from a hydrogen atom, a C₁-C₃-alkoxy group, a C₁-C₃-alkyl group, a C₁-C₃-haloalkyl group, a C₁-C₃-haloalkoxy group, and a NR¹⁷R¹⁸ group;
R¹⁴ is a hydrogen atom or a C₁-C₃-alkyl group;
R¹⁵ is independently selected from a hydrogen atom,
   a C₁-C₆-alkyl group
      which is unsubstituted or substituted with one or more substituents selected from a halogen atom, a C₁-C₃-alkyl group, a C₁-C₃-haloalkyl group, a C₁-C₃-hydroxyalkyl group, a C₁-C₃-alkoxy group, a C₁-C₃-haloalkoxy group, a heterocycloalkyl group, an aryl group, a (R¹⁹)-(heterocycloalkylene)-(arylene)-O— group, a (heterocycloalkyl)-(arylene)-O— group, an aryl-O— group, an aryl-(C₁-C₃-alkylene)-O— group, a (R²⁰)—S(O)₂-arylene-O— group, a (R²⁰)S(O)₂-heterocycloalkylene-arylene-O— group, an aryl-heteroarylene-O— group, an aryl-heteroarylene-O—(C₁-C₃-alkylene)- group, a heterocycloalkyl-(C₁-C₃-alkylene)-C(O)— group, a heterocycloalkyl-NH—C(O)— group, an aryl-(C₁-C₃-alkylene)-NH—C(O)— group, a heterocycloalkylene-(C₁-C₃-alkylene)-S(O)₂— group, and a heterocycloalkylene-heteroarylene-S(O)₂— group;
   a C₁-C₃-alkylene-C(O)— group,
   a heterocycloalkyl-(C₁-C₃-alkylene)-C(O)— group,
   a heterocycloalkyl-(C₁-C₃-alkylene)-S(O)₂— group,
   a heterocyclyl-NH—C(O)— group,
   a heterocycloalkyl-(C₁-C₃-alkylene)-NH—C(O)— group,
   an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group,
   a heterocycloalkyl-heteroarylene-S(O)₂— group,
   a phenyl group,
   a group

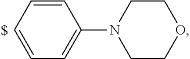

a group

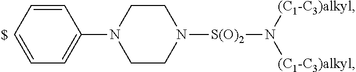

and
a group

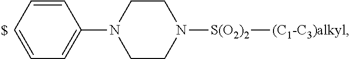

where $ is the point of attachment to the nitrogen atom, to which R¹⁵ is attached;
R¹⁶ is a pharmaceutically acceptable anion;
R¹⁷ and R¹⁸ are each independently selected from a hydrogen atom, a C₁-C₆-alkyl group, a C₁-C₆-haloalkyl group, a C₃-C₅-cycloalkyl group, a C₁-C₃-alkyl-C(O)— group, a C₁-C₃-alkylS(O)₂— group, and a C₁-C₃-alkyl-O—C(═O)— group;
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
R²⁰ is selected from a C₁-C₃-alkyl group, a C₃-C₆-cycloalkyl group, and a NR²¹R²² group; and
R²¹ and R²² are independently selected from a hydrogen atom or a C₁-C₆-alkyl group;
or a tautomer, an N-oxide, or a salt thereof, or a salt of a tautomer, or a salt of an N-oxide, or a mixture of same.

### DETAILED DESCRIPTION

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “unsubstituted or substituted” means that the number of substituents can be equal to or different from zero. Unless otherwise indicated, it is possible that substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two, three, four or five identical or different substituents, particularly with one, two or three substituents.

Oxo, an oxo group or an oxo substituent means a doubly attached oxygen atom ═O. Oxo may be attached to atoms of suitable valency, for example to a saturated carbon atom or to a sulfur atom. For example, but without limitation, one oxo group is can be attached to a carbon atom, resulting in the formation of a carbonyl group C(═O), or two oxo groups are can be attached to one sulfur atom, resulting in the formation of a sulfonyl group —S(═O)₂. The term “ring substituent” means a substituent attached to an aromatic or nonaromatic ring which replaces an available hydrogen atom on the ring.

Should a composite substituent be composed of more than one parts, e.g., (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of” but does not have to be the scope indicated by “consisting of.

If within the present text any item is referred to as “as mentioned herein”, it means that it may be mentioned anywhere in the present text.

If within the present text any item is referred to as “supra” within the description it indicates any of the respective disclosures made within the specification in any of the preceding pages, or above on the same page.

If within the present text any item is referred to as “infra” within the description it indicates any of the respective disclosures made within the specification in any of the subsequent pages, or below on the same page.

The terms as mentioned in the present text have the following meanings:

The term “halogen atom” means a fluorine, chlorine, bromine or iodine atom, particularly a fluorine, chlorine or bromine atom.

The term “C₁-C₈-alkyl-” means a linear or branched, saturated hydrocarbon group having 1, 2, 3, 4, 5, 6, 7 or 8 carbon atoms, e.g., a methyl-, ethyl-, propyl-, iso-propyl-, n-butyl-, iso-butyl-, sec-butyl-, tert-butyl-, n-pentyl-, iso-pentyl-, 2-methylbutyl-, 1-methylbutyl-, 1-ethylpropyl-, 1,2-dimethylpropyl-, neo-pentyl-, 1,1-dimethylpropyl-, n-hexyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, 1,2-dimethylbutyl-, n-heptyl-, 5-methylhexyl-, 4-methylhexyl-, 2-methylhexyl-, 1-methylhexyl-, 2-ethylpentyl-, 1-ethylpentyl-, 3,3-dimethylpentyl-, 2,2-dimethylpentyl-, 1,1-dimethylpentyl-, 2,3-dimethylpentyl-, 1,3-dimethylpentyl-, 1,2-dimethylpentyl-, n-octyl-, 6-methylheptyl-, 4-methylheptyl-, 2-methylheptyl-, 1-methylheptyl-, 2-ethylhexyl-, 1-ethylhexyl-, 3,3-dimethylhexyl-, 2,2-dimethylhexyl-, 1,1-dimethylhexyl-, 2,3-dimethylhexyl-, 1,3-dimethylhexyl-, 1,2-dimethylhexyl- group, or an isomer thereof. Preferably, said group has 1, 2, 3, 4, 5 or 6 carbon atoms (“C₁-C₆-alkyl-”), e.g., a methyl-, ethyl-, n-propyl-, iso-propyl-, n-butyl-, iso-butyl-, sec-butyl-, tert-butyl-, n-pentyl-, iso-pentyl-, 2-methylbutyl-, 1-methylbutyl-, 1-ethylpropyl-, 1,2-dimethylpropyl-, neo-pentyl-, 1,1-dimethylpropyl-, n-hexyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl- or a 1,2-dimethylbutyl group, or an isomer thereof. More preferably, said group has 1, 2, 3 or 4 carbon atoms (“C₁-C₄-alkyl-”), e.g., a methyl-, ethyl-, n-propyl-, iso-propyl-, n-butyl-, iso-butyl-, sec-butyl- or a tert-butyl- group, 1, 2 or 3 carbon atoms (“C₁-C₃-alkyl-”), e.g., a methyl-, ethyl-, n-propyl- or iso-propyl group, or 1 or 2 carbon atoms (“C₁-C₂-alkyl-”), e.g., a methyl group, an ethyl group.

The same definitions can be applied should the alkyl group be placed within a chain as a bivalent “C₁-C₆-alkylene” moiety. All names as mentioned above then will bear an “ene” added to the end, thus e.g., a “pentyl” becomes a bivalent “pentylene” group. In addition, the term “C₁-C₆-heteroalkyl” refers to a C₁-C₆-alkyl group in which one or more of the carbon atoms have been replaced with an atom selected from N, O, S, or P, which are substituted as mentioned herein to satisfy atom valency requirements.

The term “C₂-C₆-alkylene” means a linear or branched, saturated, divalent hydrocarbon chain (or “tether”) having 2, 3, 4, 5 or 6 carbon atoms, e.g., —CH₂—CH₂— (“ethylene” or “C₂-alkylene”), —CH₂—CH₂—CH₂—, —C(H)(CH₃)—CH₂— or —C(CH₃)₂— (“propylene” or “C₃-alkylene”), or, for example —CH₂—C(H)(CH₃)—CH₂—, —CH₂—C(CH₃)₂—, —CH₂—CH₂—CH₂—CH₂— (“butylene” or “C₄-alkylene”), “C₅-alkylene”, e.g., —CH₂—CH₂—CH₂—CH₂—CH₂— (“n-pentylene”), or “—C₆-alkylene-”, e.g., —CH₂—CH₂—CH₂—CH₂—CH₂—CH₂— (“n-hexylene”) or a —C(CH₃)₂—C(CH₃)₂ group.

The term “hydroxy-(C₁-C₆-alkyl)-” means a linear or branched, saturated, hydrocarbon group in which one or more hydrogen atoms of a “C₁-C₆-alkyl-” as defined supra are each replaced by a hydroxy group, e.g., a hydroxymethyl-, 1-hydroxyethyl-, 2-hydroxyethyl-, 1,2-dihydroxyethyl-, 3-hydroxypropyl-, 2-hydroxypropyl-, 2,3-dihydroxypropyl-, 1,3-dihydroxypropan-2-yl-, 3-hydroxy-2-methyl-propyl-, 2-hydroxy-2-methyl-propyl-, or a 1-hydroxy-2-methyl-propyl- group. Particularly the hydroxyalkyl group means a linear or branched, saturated, monovalent hydrocarbon group has 1, 2 or 3 carbon atoms in which 1 hydrogen atom is replaced with a hydroxy group e.g. a hydroxymethyl-, 1-hydroxyethyl-, 2-hydroxyethyl-, 3-hydroxypropyl-, 2-hydroxypropyl-, 1-hydroxypropyl-, 2-hydroxy-2-methyl-ethyl group.

The term “C₁-C₆-haloalkyl” means a linear or branched, saturated, monovalent hydrocarbon group in which the term “C₁-C₆-alkyl” is as defined supra and in which one or more of the hydrogen atoms are replaced, identically or differently, with a halogen atom. Preferably, said halogen atom is a fluorine atom. Said C₁-C₆-haloalkyl, particularly a C₁-C₃-haloalkyl group is, for example, fluoromethyl-, difluoromethyl-, trifluoromethyl-, 2-fluoroethyl-, 2,2-difluoroethyl-, 2,2,2-trifluoroethyl-, pentafluoroethyl-, 3,3,3-trifluoropropyl- or a 1,3-difluoropropan-2-yl group.

The term “C₁-C₆-alkoxy” means a linear or branched, saturated, monovalent group of formula (C₁-C₆-alkyl)-O—, in which the term “C₁-C₆-alkyl” group is as defined supra, e.g. methoxy-, ethoxy-, n-propoxy-, isopropoxy-, n-butoxy-, sec-butoxy-, isobutoxy-, tert-butoxy-, pentyloxy-, isopentyloxy- or a n-hexyloxy group, or an isomer thereof.

The term “C₁-C₆-alkylthio” or “C₁-C₆-thioalkyl” means a linear or branched, saturated, monovalent group of formula (C₁-C₆-alkyl)-S—, in which the term “C₁-C₆-alkyl” is as defined supra, e.g. methylthio-, ethylthio-, n-propylthio-, isopropylthio-, n-butylthio-, sec-butylthio-, isobutylthio-, tert-butylthio-, pentylthio-, isopentylthio- or a n-hexylthio group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Preferably, said halogen atom in “C₁-C₆-haloalkoxy-” is fluorine, resulting in a group referred herein as “C₁-C₆-fluoroalkoxy-”. Representative C₁-C₆-fluoroalkoxy- groups include, for example, —OCF₃, —OCHF₂, —OCH₂F, —OCF₂CF₃ and —OCH₂CF₃.

The term “C₁-C₆-haloalkylthio” or “C₁-C₆-halothioalkyl” or “C₁-C₆-haloalkyl-S—” means a linear or branched, saturated, monovalent C₁-C₆-alkylthio group, as defined supra, in which one or more of the hydrogen atoms is replaced, identically or differently, with a halogen atom. Preferably, said halogen atom in “C₁-C₆-haloalkylthio-” is fluorine.

The term “C₂-C₆-alkenyl-” means a linear or branched, monovalent hydrocarbon group, which contains one or more double bonds and which has 2, 3, 4, 5 or 6 carbon atoms, preferably 2, 3 or 4 carbon atoms (“C₂-C₄-alkenyl-”) or 2 or 3 carbon atoms (“C₂-C₃-alkenyl-”), it being understood that in the case in which said alkenyl- group contains more than one double bond, then said double bonds may be isolated from, or conjugated with, each other. Representative alkenyl groups include, for example, an ethenyl-, prop-2-enyl-, (E)-prop-1-enyl-, (Z)-prop-1-enyl-, iso-propenyl-, but-3-enyl-, (E)-but-2-enyl-, (Z)-but-2-enyl-, (E)-but-1-enyl-, (Z)-but-1-enyl-, 2-methylprop-2-enyl-, 1-methylprop-2-enyl-, 2-methylprop-1-enyl-, (E)-1-methylprop-1-enyl-, (Z)-1-methylprop-1-enyl-, buta-1,3-dienyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, (E)-3,3-dimethylprop-1-enyl-, (Z)-3,3-dimethylprop-1-enyl-, penta-1,4-dienyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, hexa-1,5-dienyl- and a 1-(1,1-dimethylethyl-)ethenyl group. Particularly, said group is an ethenyl- or a prop-2-enyl group.

The same definitions can be applied should the alkenyl group be placed within a chain as a bivalent “C₂-C₆-alkenylene” moiety. All names as mentioned above then will bear a “ene” added to their end, thus e.g., a “pentenyl” becomes a bivalent “pentenylene” group.

The term “C₂-C₆-haloalkenyl-” means a linear or branched hydrocarbon group in which one or more of the hydrogen atoms of a “C₂-C₆-alkenyl-” as defined supra are each replaced, identically or differently, by a halogen atom. Preferably, said halogen atom is fluorine, resulting in a group referred herein as “C₂-C₆-fluoroalkenyl-”. Representative C₂-C₆-fluoroalkenyl- groups include, for example, —CH═CF₂, —CF═CH₂, —CF═CF₂, —C(CH₃)═CF₂, —CH═C(F)—CH₃, —CH₂—CF═CF₂ and —CF₂—CH═CH₂.

The term “C₂-C₆-alkynyl-” means a linear or branched, monovalent hydrocarbon group which contains one or more triple bonds, and which contains 2, 3, 4, 5 or 6 carbon atoms, preferably 2, 3 or 4 carbon atoms (“C₂-C₄-alkynyl-”) or 2 or 3 carbon atoms (“C₂-C₃-alkynyl-”). Representative C₂-C₆-alkynyl- groups include, for example, an ethynyl-, prop-1-ynyl-, prop-2-ynyl-, but-1-ynyl-, but-2-ynyl-, but-3-ynyl-, pent-1-ynyl-, pent-2-ynyl, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methyl-pent-4-ynyl-, 2-methylpent-3-ynyl-, 1-methylpent-3-ynyl-, 4-methylpent-2-ynyl-, 1-methyl-pent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl- and a 3,3-dimethylbut-1-ynyl- group. Particularly, said alkynyl- group is an ethynyl-, a prop-1-ynyl- or a prop-2-ynyl group.

The term “C₃-C₇-cycloalkyl-” means a saturated mono- or bicyclic hydrocarbon ring which contains 3, 4, 5, 6, or 7 carbon atoms (“C₃-C₇-cycloalkyl-”). Said C₃-C₇-cycloalkyl- group may be, for example, a monocyclic hydrocarbon ring, e.g., a cyclopropyl-, cyclobutyl-, cyclopentyl-, cyclohexyl- or cycloheptyl- group, or a bicyclic hydrocarbon ring, such as a decalinyl group. Particularly, said hydrocarbon ring is monocyclic and contains 3, 4, 5, 6 or 7 carbon atoms (“C₃-C₇-cycloalkyl-”), e.g., a cyclopropyl-, cyclobutyl-, cyclopentyl-, cyclohexyl- or cycloheptyl- group, or 3, 4, 5 or 6 carbon atoms (“C₃-C₆-cycloalkyl-”), e.g., a cyclopropyl-, cyclobutyl-, cyclopentyl- or cyclohexyl- group or even 3, 4, or 5 carbon atoms (“C₃-C₆-cycloalkyl-”), e.g., a cyclopropyl-, cyclobutyl- or a cyclopentyl group. A cycloalkyl group may be unsubstituted or substituted as defined at the respective part wherein such term is used.

The term “1,2-cyclopropylene is used in the definition of —R⁶-R⁷— and means

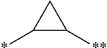

where * and ** are the points of attachment to the adjacent carbon atoms of the alkenylene group.

The term “C₄-C₈-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₈-cycloalkenyl group is for example, a monocyclic hydrocarbon ring, e.g., a cyclobutenyl-, cyclopentenyl-, cyclohexenyl-, cycloheptenyl- or a cyclooctenyl group, or a bicyclic hydrocarbon ring, e.g., a bicyclo[2.2.1]hept-2-enyl- or a bicyclo[2.2.2]oct-2-enyl group.

If the term “heterocycloalkyl” is used without specifying a number of atoms it is meant to be a “4- to 10-membered heterocycloalkyl-” group, more particularly a 5- to 6-membered heterocycloalkyl group. The term “4- to 10-membered heterocycloalkyl-” means a saturated mono- or bicyclic hydrocarbon ring which contains 3, 4, 5, 6, 7, 8 or 9 carbon atoms, and which contains 1, 2, 3 or 4 heteroatoms which may be identical or different, said heteroatoms are preferably selected from oxygen, nitrogen or sulfur, and wherein carbon atoms and heteroatoms add up to 4, 5, 6, 7, 8, 9 or 10 ring atoms in total, it being possible for said heterocycloalkyl- group to be attached to the rest of the molecule via any one of the carbon atoms or, if present, a nitrogen atom. “Heterospirocycloalkyl-”, “heterobicycloalkyl-” and “bridged heterocycloalkyl-”, as defined infra, are also included within the scope of this definition.

Preferably, said “4-membered to 10-membered heterocycloalkyl-” group is monocyclic and contains 3, 4, 5 or 6 carbon atoms, and one or two of the above-mentioned heteroatoms, adding up to 4, 5, 6 or 7 ring atoms in total (a “4-membered to 7-membered monocyclic heterocycloalkyl-”), or contains 3, 4 or 5 carbon atoms, and one or two of the above-mentioned heteroatoms, adding up to 4, 5 or 6 ring atoms in total (a “4-membered to 6-membered monocyclic heterocycloalkyl-”), or contains 3, 4 or 5 carbon atoms, and one or two of the above-mentioned heteroatoms, adding up to 5 or 6 ring atoms in total (a “5-membered to 6-membered monocyclic heterocycloalkyl-”); it being possible for said heterocycloalkyl- group to be attached to the rest of the molecule via any one of the carbon atoms or the nitrogen atoms, if present.

Exemplarily, without being limited thereto, said “4-membered to 7-membered monocyclic heterocycloalkyl-”, can be a 4-membered ring, a “4-membered heterocycloalkyl-” group, such as an azetidinyl- or an oxetanyl group; or a 5-membered ring, a “5-membered heterocycloalkyl-” group, such as a tetrahydrofuranyl-, dioxolinyl-, pyrrolidinyl-, imidazolidinyl-, pyrazolidinyl- or a pyrrolinyl group; or a 6-membered ring, a “6-membered heterocycloalkyl-” group, such as a tetrahydropyranyl-, piperidinyl-, morpholinyl-, 3-oxomorpholin-4-yl, dithianyl-, thiomorpholinyl- or a piperazinyl group; or a 7-membered ring, a “7-membered heterocycloalkyl-” group, such as an azepanyl-, diazepanyl- or an oxazepanyl group, for example. The heterocycloalkyl groups may be one or more times substituted with C₁-C₃-alkyl, C₁-C₃-alkoxy, hydroxy, halogen or a carbonyl group.

The term “heterocycloalkenyl” or “5- to 7-membered heterocycloalkenyl” means a monocyclic, unsaturated, non-aromatic heterocycle with 5, 6, or 7 ring atoms in total, which contains one or two double bonds and one or two identical or different ring heteroatoms from the series: N, O, S; it being possible for said heterocycloalkenyl group to be attached to the rest of the molecule via any one of the carbon atoms or, if present, a nitrogen atom.

Said heterocycloalkenyl group is, for example, a 4H-pyranyl-, 3,6-dihydro-2H-pyran-4-yl-, 2H-pyranyl-, dihydropyridinyl-, tetrahydropyridinyl-, 2-oxopyridin-1(2H)-yl-, 2,5-dihydro-1H-pyrrolyl-, [1,3]dioxolyl-, 4H-[1,3,4]thiadiazinyl-, 2,5-dihydrofuranyl-, 2,3-dihydrofuranyl-, 2,5-dihydrothiophenyl-, 2,3-dihydrothiophenyl-, 4,5-dihydrooxazolyl- or a 4H-[1,4]thiazinyl group. Those heterocycloalkenyl groups may be substituted with a hydroxy group or a methoxy group.

The term “fused heterocycloalkyl” or “heterobicycloalkyl-” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl or “heterobicycloalkyl-” group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]-nonyl or azabicyclo[4.4.0]decyl.

The term “aryl” means a phenyl-, naphthyl-, 5,6-dihydronaphthyl-, 7,8-dihydronaphthyl-, 5,6,7,8-tetrahydronaphthyl-, indanyl-, or an indenyl group, which is unsubstituted or substituted with one, two, three, four or five substituents, each substituent independently selected from halogen, cyano, C₁-C₃-alkyl, C₁-C₃-haloalkyl, C₁-C₃-alkoxy, C₁-C₃-thioalkyl, C₁-C₃-haloalkoxy, C₁-C₃-halothioalkyl, C₃-C₅-cycloalkyl, particularly halogen, C₁-C₃-alkyl, C₁-C₃-haloalkyl, C₁-C₃-alkoxy, C₁-C₃-haloalkoxy, particularly halogen, C₁-C₃-alkyl and C₁-C₃-haloalkyl.

The term “heteroaryl-” means a monocyclic, bicyclic or tricyclic aromatic ring system having 5, 6, 7, 8, 9, 10, 11, 12, 13 or 14 ring atoms (a “5- to 14-membered heteroaryl-” group), preferably 5, 6, 9 or 10 ring atoms and which contains 1, 2, 3 or 4 heteroatoms which may be identical or different, said heteroatoms being selected from oxygen, nitrogen and sulfur. Said heteroaryl- group can be a 5-membered heteroaryl group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl- or a tetrazolyl group; or a 6-membered heteroaryl group, such as, for example, a pyridyl-, pyridazinyl-, pyrimidyl-, pyrazinyl- or a triazinyl group; or a benzo-fused 5-membered heteroaryl- group, such as, for example, a benzofuranyl-, benzothienyl-, benzoxazolyl-, benzisoxazolyl-, benzimidazolyl-, benzothiazolyl-, benzotriazolyl-, indazolyl-, indolyl- or a isoindolyl group; or a benzo-fused 6-membered heteroaryl group, such as, for example, a quinolinyl-, quinazolinyl-, isoquinolinyl-, cinnolinyl-, phthalazinyl- or quinoxalinyl-; or another bicyclic group, such as, for example, indolizinyl-, purinyl- or a pteridinyl group.

Preferably, “heteroaryl-” is a monocyclic aromatic ring system having 5 or 6 ring atoms and which contains at least one heteroatom, if more than one, they may be identical or different, said heteroatom being selected from oxygen, nitrogen and sulfur, a (“5- to 6-membered monocyclic heteroaryl-”) group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, tetrazolyl-, pyridyl-, pyridazinyl-, pyrimidyl-, pyrazinyl- or a triazinyl group.

In general, and unless otherwise mentioned, said heteroaryl- groups include all the possible isomeric forms thereof, e.g., the positional isomers thereof. Thus, for some illustrative non-restricting example, the term pyridyl- includes pyridin-2-yl-, pyridin-3-yl- and pyridin-4-yl-; the term thienyl- includes thien-2-yl- and thien-3-yl-. Furthermore, said heteroaryl- groups can be attached to the rest of the molecule via any one of the carbon atoms, or, if applicable, a nitrogen atom, e.g., a pyrrol-1-yl-, a pyrazol-1-yl- or an imidazol-1-yl- group.

In general, and unless otherwise mentioned, the heteroaryl or heteroarylene groups include all possible isomeric forms thereof, e.g., tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl, and heteroarylene group may be inserted into a chain also in the inverse way such as e.g. a 2,3-pyridinylene includes pyridine-2,3-yl as well as pyridine-3,2-yl.

Particularly, the heteroaryl group is a pyridyl- or pyrimidyl group or a imidazolyl group. including a hydroxy substitution of the pyridyl group leading e.g. to a 2-hydroxy-pyridine which is the tautomeric form to a 2-oxo-2(1H)-pyridine. In some embodiments, the heteroaryl group is an oxazolyl group.

The term “C₁-C₆”, as used throughout this text, e.g., in the context of the definition of “C₁-C₆-alkyl-”, “C₁-C₆-haloalkyl-”, “C₁-C₆-alkoxy-” or “C₁-C₆-haloalkoxy-” is to be understood as meaning an alkyl group having a whole number of carbon atoms from 1 to 6, i.e., 1, 2, 3, 4, 5 or 6 carbon atoms. It is to be understood further that said term “C₁-C₆” is to be interpreted as disclosing any sub-range comprised therein, e.g. C₁-C₆, C₂-C₅, C₃-C₄, C₁-C₂, C₁-C₃, C₁-C₄, C₁-C₅, C₁-C₆; preferably C₁-C₂, C₁-C₃, C₁-C₄, C₁-C₅, C₁-C₆ more preferably C₁-C₄ in the case of “C₁-C₆-haloalkyl-” or “C₁-C₆-haloalkoxy-” even more preferably C₁-C₂.

Similarly, as used herein, the term “C₂-C₆”, as used throughout this text, e.g., in the context of the definitions of “C₂-C₆-alkenyl-” and “C₂-C₆-alkynyl-”, is to be understood as meaning an alkenyl- group or an alkynyl group having a whole number of carbon atoms from 2 to 6, i.e., 2, 3, 4, 5 or 6 carbon atoms. It is to be understood further that said term “C₂-C₆” is to be interpreted as disclosing any sub-range comprised therein, e.g., C₂-C₆, C₃-C₅, C₃-C₄, C₂-C₃, C₂-C₄, C₂-C₅; preferably C₂-C₃.

Further, as used herein, the term “C₃-C₇”, as used throughout this text, e.g., in the context of the definition of “C₃-C₇-cycloalkyl-”, is to be understood as meaning a cycloalkyl- group having a whole number of carbon atoms of 3 to 7, i.e., 3, 4, 5, 6 or 7 carbon atoms. It is to be understood further that said term “C₃-C₇” is to be interpreted as disclosing any sub-range comprised therein, e.g., C₃-C₆, C₄-C₅, C₃-C₅, C₃-C₄, C₄-C₆, C₅-C₇; preferably C₃-C₆.

As used herein, the term “leaving group” refers to an atom or a group of atoms that is displaced in a chemical reaction as stable species taking with it the bonding electrons, e.g., typically forming an anion. Preferably, a leaving group is selected from the group comprising: halo, in particular a chloro, bromo or iodo, (methylsulfonyl)oxy-, [(4-methylphenyl)sulfonyl]oxy-, [(trifluoromethyl)sulfonyl]oxy-, [(nonafluorobutyl)sulfonyl]oxy-, [(4-bromophenyl)sulfonyl]oxy-, [(4-nitrophenyl)sulfonyl]oxy-, [(2-nitrophenyl)sulfonyl]oxy-, [(4-isopropylphenyl)sulfonyl]oxy-, [(2,4,6-triisopropylphenyl)sulfonyl]oxy-, [(2,4,6-trimethylphenyl)sulfonyl]oxy-, [(4-tert-butylphenyl)sulfonyl]oxy-, (phenylsulfonyl)oxy-, and a [(4-methoxyphenyl)sulfonyl]oxy group.

As used herein, the term “protective group” is a protective group attached to an oxygen or nitrogen atom in intermediates used for the preparation of compounds of the general formula (I). Such groups are introduced e.g., by chemical modification of the respective hydroxy or amino group in order to obtain chemoselectivity in a subsequent chemical reaction. Protective groups for hydroxy and amino groups are described for example in T. W. Greene and P. G. M. Wuts in Protective Groups in Organic Synthesis, 4^{th} edition, Wiley 2006; more specifically, protective groups for amino groups can be selected from substituted sulfonyl groups, such as a mesyl-, tosyl- or a phenylsulfonyl group, acyl groups such as a benzoyl-, acetyl- or a tetrahydropyranoyl group, or carbamate based groups, such as a tert-butoxycarbonyl group (Boc). Protective groups for hydroxy groups can be selected from acyl groups such as a benzoyl-, acetyl, pivaloyl- or a tetrahydropyranoyl group, or can include silicon, as in e.g., a tert-butyldimethylsilyl-, tert-butyldiphenylsilyl-, triethylsilyl- or a triisopropylsilyl group.

The term “substituent” refers to a group “substituted” on, e.g., an alkyl-, haloalkyl-, cycloalkyl-, heterocyclyl-, heterocycloalkenyl-, cycloalkenyl-, aryl-, or a heteroaryl group at any atom of that group, replacing one or more hydrogen atoms therein. In one aspect, the substituent(s) on a group are independently any one single, or any combination of two or more of the permissible atoms or groups of atoms delineated for that substituent. In another aspect, a substituent may itself be substituted with any one of the above substituents. Further, as used herein, the phrase “optionally substituted” means unsubstituted (e.g., substituted with an H) or substituted.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon.

By “subject” is meant a mammal, including, but not limited to, a human or non-human mammal, such as a bovine, equine, canine, ovine, rodent, or feline.

It is possible for the compounds of general formula (I) to exist as isotopic variants. The invention therefore includes one or more isotopic variant(s) of the compounds of general formula (I), particularly deuterium-containing compounds of general formula (I).

The invention also includes all suitable isotopic variations of a compound of the invention.

The term “isotopic variant” of a compound or a reagent is defined as a compound exhibiting an unnatural proportion of one or more of the isotopes that constitute such a compound.

The expression “unnatural proportion” in relation to an isotope means a proportion of such isotope which is higher than its natural abundance. The natural abundances of isotopes to be applied in this context are described in “Isotopic Compositions of the Elements 1997”, Pure Appl. Chem., 70(1), 217-235, 1998.

An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

With respect to the treatment and/or prophylaxis of the disorders specified herein, the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful, e.g., in drug and/or substrate tissue distribution studies. These isotopes are particularly preferred for the ease of their incorporation and detectability. Positron-emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g., Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g., Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g., lower number of doses or lower dosage to achieve the desired effect) and/or may produce lower metabolite loads.

A compound of general formula (I) may have multiple potential sites of vulnerability to metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g., cytochrome P₄₅₀.

For example, in some embodiments, the present invention concerns a deuterium-containing compound of general formula (I), e.g.:

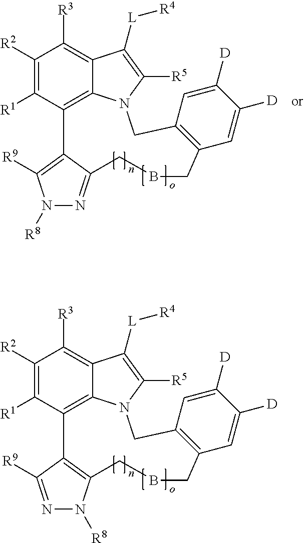

Such deuterium-containing compounds can be prepared by methods well-known to the person skilled in the art. Particularly, such deuterium-containing compounds can be prepared from the corresponding olefins, which are available by methods known to the person skilled in the art, such as ring closing metathesis reactions, as discussed e.g., in the general description of the synthesis of compounds of general formula (I), infra, in the context of Schemes 2c and 2j, respectively.

Where the plural form of the word compounds, salts, polymorphs, hydrates, solvates and the like, is used herein, this is taken to mean also a single compound, salt, polymorph, isomer, hydrate, solvate or the like. The terms “a” or “an,” as used in herein means one or more.

By “stable compound’ or “stable structure” is meant a compound that is sufficiently robust to survive isolation to a useful degree of purity from a reaction mixture, and formulation into an efficacious therapeutic agent.

Compounds of the present invention, as well as the corresponding macrocyclic intermediates of formula (II), are typically chiral merely as a result of restricted rotation around at least one single bond, which is due to limited conformational flexibility of their macrocyclic core as a whole or even of open chain precursors. Hence, compounds of the present invention as well as the corresponding macrocyclic intermediates of formula (II), can exist as atropisomers. Atropisomers represent a subclass of conformers which arise from restricted rotation around a single bond. The conformers (called atropisomers) can be isolated as separated species (IUPAC Gold book, http://goldbook.iupac.org/A00511.html; Pure and Appl. Chem., 2009, 68, 2193-2222). This induced chirality belongs to the axial type of chirality. The compounds of the present invention as well as the corresponding macrocyclic intermediates of formula (II), furthermore optionally contain one or more asymmetric centers, depending upon the location and nature of the various substituents desired. It is possible that one or more asymmetric carbon atoms are present in the (R) or (S) configuration, which can result in racemic mixtures in the case of a single asymmetric center, and in diastereomeric mixtures in the case of multiple asymmetric centers. Hence, compounds of the present invention, as well as the corresponding macrocyclic intermediates of formula (II), featuring the abovementioned atropisomerism and an additional asymmetric centre can also exist as diasteromeric mixtures as described supra.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

If only one isomer (enantiomer) displays the desired biological activity, and the second isomer (enantiomer) is inactive, the preferred isomer is the one which produces the more desirable biological activity. Should one isomer (enantiomer/diastereomer) display better activity than the other isomer (enantiomer/diastreromer) the preferred isomer is the one which produces the better biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials, enantioselective catalytic reactions, and other suitable methods.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, in any ratio. Isolation of a single stereoisomer, e.g., a single enantiomer or a single diastereomer, of a compound of the present invention may be achieved by any suitable method, such as chromatography, especially chiral chromatography, for example.

Further, it is possible for the compounds of the present invention to exist as tautomers. For example, any compound of the present invention which contains an pyrazol moiety as a heteroaryl group for example can exist as a 1H tautomer, or a 2H tautomer, or even a mixture in any amount of the two tautomers, namely:

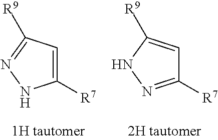

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also includes useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention form a crystal that contains molecules of polar solvents, in particular water, methanol or ethanol, for example, as structural element of the crystal lattice of the compounds. The molecules of polar solvents, in particular water, may be present in a stoichiometric or non-stoichiometric ratio with the molecules of the compound. In the case of stoichiometric solvates, e.g., a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g., as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19. It includes any physiologically acceptable salt as referred to below.

Physiologically acceptable salts of the compounds according to the invention encompass acid addition salts of mineral acids, carboxylic acids and sulfonic acids, for example salts of hydrochloric acid, hydrobromic acid, hydroiodic acid, sulfuric acid, bisulfuric acid, phosphoric acid, nitric acid or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalenedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

A “pharmaceutically acceptable anion” refers to the deprotonated form of a conventional acid, such as, for example, a hydroxide, a carboxylate, a sulfate, a halide, a phosphate, or a nitrate.

Physiologically acceptable salts of the compounds according to the invention also comprise salts of conventional bases, such as, by way of example and by preference, alkali metal salts (for example lithium, sodium and potassium salts), alkaline earth metal salts (for example calcium, strontium and magnesium salts) and ammonium salts derived from ammonia or organic amines with 1 to 16 C atoms, such as, by way of example and by preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base, and 1-amino-2,3,4-butanetriol.

Additionally, the compounds according to the invention may form salts with a quaternary ammonium ion obtainable, e.g., by quaternisation of a basic nitrogen-containing group with agents such as lower alkylhalides such as methyl-, ethyl-, propyl-, and butylchlorides, -bromides and -iodides; dialkylsulfates such as dimethyl-, diethyl-, dibutyl- and diamylsulfates, long chain halides such as decyl-, lauryl-, myristyl- and stearylchlorides, -bromides and -iodides, aralkylhalides such as benzyl- and phenethylbromides and others. Examples of suitable quaternary ammonium ions are tetramethylammonium, tetraethylammonium, tetra(n-propyl)ammonium, tetra (n-butyl)ammonium, or N-benzyl-N,N,N-trimethylammonium.

The present invention includes all possible salts of the compounds of the present invention as single salts, or as any mixture of said salts, in any ratio.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

Solvates and hydrates of disclosed intermediates or example compounds, or salts thereof, which have been obtained, by the preparation and/or purification processes described herein, may be formed in any ratio.

Furthermore, the present invention includes all possible crystalline forms, or polymorphs, of the compounds of the present invention, either as a single polymorph, or as a mixture of more than one polymorph, in any ratio.

Moreover, the present invention also includes prodrugs of the compounds according to the invention. The term “prodrugs” designates compounds which themselves can be biologically active or inactive, but are converted (for example metabolically or hydrolytically) into compounds according to the invention during their residence time in the body. For example, a prodrug may be in the form of an in vivo hydrolysable ester of the specified compound. Derivatives of the compounds of formula (I) and the salts thereof which are converted into a compound of formula (I) or a salt thereof in a biological system (bioprecursors or pro-drugs) are covered by the invention. Said biological system may be, for example, a mammalian organism, particularly a human subject. The bioprecursor is, for example, converted into the compound of formula (I) or a salt thereof by metabolic processes.

### Further Embodiments of the First Aspect of the Present Invention

In accordance with a further aspect, the present invention provides compounds of general formula (I): wherein
A is

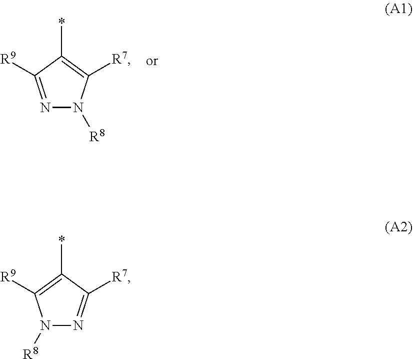

wherein R⁶ and R⁷, together with two carbon atoms of the pyrazole ring, two carbon atoms of the indole moiety and the nitrogen atom to which R⁶ is attached, form a 9- to 13-membered ring and * is the point of attachment of these moieties to the indole carbon atom bearing the A substituent;
R¹ and R² are each independently selected from a hydrogen atom, a halogen atom, a cyano group, a C₁-C₃-alkyl group, and a C₁-C₃-alkoxy group;
R³ is selected from a hydrogen atom, a halogen atom, a cyano group, a C₁-C₃-alkyl group, a C₁-C₃-haloalkyl group, a C₁-C₃-alkoxy group and a C₃-C₅-cycloalkyl group;
R⁴ is selected from an aryl group and a heteroaryl group, each of which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group and a C₃-C₅-cycloalkyl group;
L is a group —(CH₂)ₘ-E- wherein any CH₂ group is unsubstituted or substituted with one or two substituents and each substituent is independently selected from a halogen atom, a cyano group, a hydroxyl group, a C₁-C₃-alkyl group and a C₁-C₃-alkoxy group;
E is a bond, an oxygen atom, a sulfur atom, or a —NR¹⁴— group and constitutes the connecting element to R⁴;
m is 2, 3, or 4;
R⁵ is selected from a COOH group and a

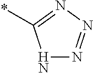 [SMILES: *c1nnn[nH]1]

group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, and a C₁-C₃-alkoxy group, wherein ^{#} is the point of attachment with the indole nitrogen atom and ^{##} is the point of attachment with the pyrazole carbon atom bearing the R⁷ substituent;
n is 1, 2, or 3;
t is 1;
r is 1, 2, or 3;
p is 1, 2, or 3;
where the integers selected for variables n, t, r, and p result in forming a 9- to 13-membered ring independently from the selection of variable A1 or A2;
B is independently selected from a —C(O)NR¹⁵— group, a —NR¹⁵C(O)— group, a —N(R¹⁵)— group, and —O—;
G is a 1,2-arylene group or
   a mono- or bicyclic heteroarylene group wherein two vicinal carbon atoms thereof are each bound to one of the adjacent alkylene groups,
   which are unsubstituted or substituted with one or more substituents independently selected from a halogen atom, a cyano group, a C₁-C₃-alkyl group, and a C₁-C₃-alkoxy group;
R⁸ is selected from a hydrogen atom, and
   a C₁-C₆-alkyl group, which is unsubstituted or substituted with one or more substituents independently selected from
      a halogen atom, a hydroxy group, a C₁-C₃-alkoxy group, a C₁-C₃-haloalkoxy group, a C₃-C₆-cycloalkyl group, a heterocycloalkyl group, and a NR²¹R²² group,
R⁹ is selected from a hydrogen atom,
   a C₁-C₄-alkyl group,
   a C₁-C₃-hydroxyalkyl group,
   a C₁-C₄-haloalkyl group,
   a C₁-C₄-haloalkyl-NH—C(O)—O—(C₁-C₃-alkylene)- group,
   a C₂-C₆-haloalkenyl group,
   a C₁-C₆-alkyl-O— group,
   a C₁-C₄-haloalkoxy group,
   a C₁-C₆-alkyl-O—(C₁-C₃-alkylene)- group,
   a (C₃-C₇)-cycloalkyl group,
   a (C₃-C₇)-cycloalkyl-O—(C₁-C₃-alkylene)- group,
   a phenyl-O—(C₁-C₃-alkylene)- group,
   a phenyl-(C₁-C₃-alkylene)-O—(C₁-C₃-alkylene)- group,
   a R¹⁹-(phenylene)-(C₁-C₃-alkylene)-O—(C₁-C₃-alkylene)- group,
   a R¹⁹-(phenylene)-O—(C₁-C₃-alkylene)- group,
   a R¹⁹-(phenylene)-(heteroarylene)-O—(C₁-C₃-alkylene)- group,
   a (R¹⁹)-(heterocycloalkylene)-(C₁-C₃-alkylene)- group,
   a (R¹⁹)-(heterocycloalkylene)-(phenylene)-O—(C₁-C₃-alkylene)- group,
   a (heterocycloalkenyl)-(phenylene)-O—(C₁-C₃-alkylene)- group,
   a (R¹⁹)-(heteroarylene)-(C₁-C₃-alkylene)-O—(C₁-C₃-alkylene)- group,
   a (R¹⁹)-(heteroarylene)-(phenylene)-O—(C₁-C₃-alkylene)- group,
   a (R²⁰)—S(O)₂-(phenylene)-O—(C₁-C₃-alkylene)- group,
   a (R²⁰)—S(O)₂—NH-(phenylene)-O—(C₁-C₃-alkylene)- group,
   a (R²⁰)—S(O)₂—NH-(phenylene)-O—(C₁-C₃-alkylene)- group,
   a (R²⁰)—S(O)₂—N(C₁-C₆-alkyl)-(phenylene)-O—(C₁-C₃-alkylene)- group,
   a (R²⁰)—S(O)₂-(heterocycloalkylene)-(phenylene)-O—(C₁-C₃-alkylene)- group,
   a (R¹⁹)-(heterocycloalkylene)-(heteroarylene)-O—(C₁-C₃-alkylene)- group,
   a (R²⁰)-(heteroarylene)-O—(C₁-C₃-alkylene)- group,
   a (R¹⁹)-(heteroarylene)-O—(C₁-C₃-alkylene)- group,
   a (R²⁰)—S(O)₂-(heteroarylene)-O—(C₁-C₃-alkylene)- group,
   a (R²⁰)—S(O)₂-(heterocycloalkylene)-(heteroarylene)-O—(C₁-C₃-alkylene)- group,
   a NR²¹R²²—(C₁-C₃-alkylene)- group,
   a (C₁-C₃-alkyl)-NH—(C₁-C₃-alkylene)- group,
   a (C₁-C₃-haloalkyl)-(C₁-C₃-alkylene)-NH—(C₁-C₃-alkylene)- group,
   a (C₁-C₃-haloalkyl)-NH—(C₁-C₃-alkylene)- group,
   a (C₁-C₃-alkyl)-NH—C(O)—(C₁-C₃-alkylene)- group,
   a (C₁-C₃-alkyl)-NR¹⁵—C(O)—(C₁-C₃-alkylene)- group,
   a (C₁-C₃-alkyl)-C(O)—NH—(C₁-C₃-alkylene)- group,
   a (C₁-C₃-alkyl)-C(O)—NR¹⁵—(C₁-C₃-alkylene)- group,
   a

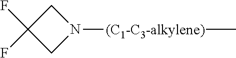

group, and a

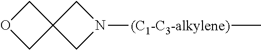

group, where the phenyl ring is unsubstituted or substituted with a halogen atom, a hydroxyl group, or a C₁-C₃-alkoxy group and
the heterocycloalkyl group is unsubstituted or substituted with an oxo (═O) group or is unsubstituted or substituted with one or more substituents independently selected from a halogen atom, a hydroxyl group, a C₁-C₃-alkyl group, and a C₁-C₃-alkoxy group,
R¹⁴ is a hydrogen atom or a C₁-C₃-alkyl group;
R¹⁵ is independently selected from a hydrogen atom,
   a C₁-C₆-alkyl group
      which is unsubstituted or substituted with one or more substituents selected from a C₁-C₃-alkyl group, a heterocycloalkyl group, and an aryl group;
   a C₁-C₃-alkylene-C(O)— group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)—,
   an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group,
   a heterocycloalkyl-heteroarylene-S(O)₂— group,
   a phenyl group,
   a group

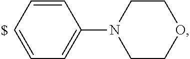

a group

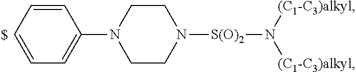

and
a group

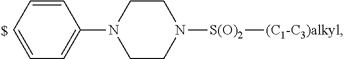

where $ is the point of attachment to the nitrogen atom, to which R¹⁵ is attached,
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
R²⁰ is selected from a C₁-C₃-alkyl group, a C₃-C₆-cycloalkyl group, and a NR²¹R²² group; and
R²¹ and R²² are independently selected from a hydrogen atom and a C₁-C₆-alkyl group;
or a tautomer, an N-oxide, or a salt thereof, or a salt of a tautomer, or a salt of an N-oxide, or a mixture of same.

In accordance with a further aspect, the present invention provides compounds of general formula (I)
wherein
A is

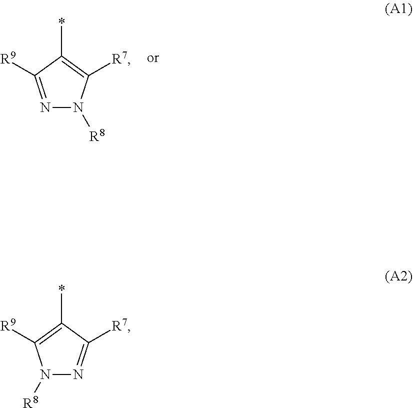

wherein R⁶ and R⁷, together with two carbon atoms of the pyrazole ring, two carbon atoms of the indole moiety and the nitrogen atom to which R⁶ is attached, form a 10- to 12-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three, substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 2, 3, or 4;
R⁵ is a COOH group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, and a C₁-C₃-alkoxy group, wherein ^{#} is the point of attachment with the indole nitrogen atom and ^{##} is the point of attachment with the pyrazole carbon atom bearing the R⁷ substituent;
n is 1 or 2;
t is 1;
r is 1 or 2;
p is 1 or 2;
where the integers selected for variables n, t, r, and p result in forming a 10- to 12-membered ring independently from the selection of variable A1 or A2;
B is selected from a —N(R¹⁵)— group and —O—,
G is a 1,2-arylene group or
   a monocyclic heteroarylene group wherein two vicinal carbon atoms thereof are each bound to one of the adjacent alkylene groups,
   which are unsubstituted or substituted with one or more substituents independently selected from a halogen atom, a cyano group, a C₁-C₃-alkyl group, and a C₁-C₃-alkoxy group;
R⁸ is selected from a hydrogen atom, and
   a C₁-C₆-alkyl group, which is unsubstituted or substituted with one or more substituents independently selected from
      a halogen atom, a hydroxy group, a C₃-C₆-cycloalkyl group and a heterocycloalkyl group;
R⁹ is selected from a hydrogen atom,
   a C₁-C₄-alkyl group,
   a C₁-C₃-hydroxyalkyl group,
   a C₁-C₄-haloalkyl group,
   a C₁-C₆-alkyl-O— group,
   a C₁-C₄-haloalkoxy group,
   a C₁-C₆-alkyl-O—(C₁-C₃-alkylene)- group,
   a (C₃-C₇)-cycloalkyl group,
   a R¹⁹-(phenylene)-O—(C₁-C₃-alkylene)- group,
   a NR²¹R²²—(C₁-C₃-alkylene)- group, and
   a (C₁-C₃-alkyl)-NH—(C₁-C₃-alkylene)- group;
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group, and
   a heterocycloalkyl-heteroarylene-S(O)₂— group;
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
and
R²¹ and R²² are independently selected from a hydrogen atom and a C₁-C₆-alkyl group;
or a tautomer, an N-oxide, or a salt thereof, or a salt of a tautomer, or a salt of an N-oxide, or a mixture of same.

In accordance with a further aspect, the present invention provides compounds of general formula (I)
wherein
A is

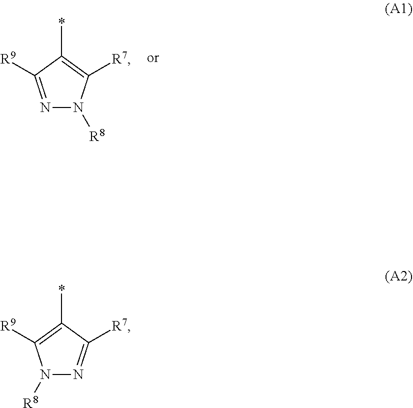

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 3;
R⁵ is a COOH group;
—R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is a 1,2-arylene group or
   a monocyclic heteroarylene group wherein two vicinal carbon atoms thereof are each bound to one of the adjacent alkylene groups,
   which are each independently unsubstituted or substituted with one or more substituents selected from a halogen atom and a C₁-C₃-alkyl group;
R⁸ is selected from a hydrogen atom, and
   a C₁-C₆-alkyl group, which is unsubstituted or substituted with a heterocycloalkyl group;
R⁹ is selected from a hydrogen atom,
   a C₁-C₄-alkyl group,
   a C₁-C₆-alkyl-O—(C₁-C₃-alkylene)- group,
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected from a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I) according to claim 1,
wherein
A is

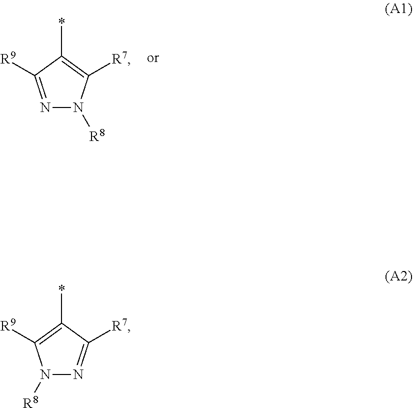

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from an aryl group and a heteroaryl group, each of which is unsubstituted or substituted with one, two, or three, substituents and each substituent is independently selected from a halogen atom and a C₁-C₃-alkyl group;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p, result in forming a 11-membered ring independently from the selection of variable A1, or A2;
B is independently selected from a —N(R¹⁵)— group and —O—;
G is an 1,2-arylene group or
   a monocyclic heteroarylene group whereby two vicinal carbon atoms thereof are each bound to one of the adjacent alkylene groups, and which each are unsubstituted;
R⁸ is selected from a hydrogen atom, a methyl group, and a —CH₂—CH₂—(N-morpholino) group;
R⁹ is selected from a hydrogen atom and a C₁-C₃-alkyl group;
R¹⁵ is a hydrogen atom,
   a C₁-C₃-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected form a halogen atom, a C₁-C₃-alkyl group and/or a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I)
wherein
A is

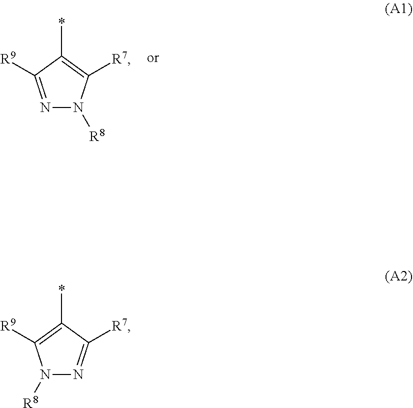

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from an aryl group and a heteroaryl group, each of which is unsubstituted or substituted with one, two, or three, substituents and each substituent is independently selected from a halogen atom and a C₁-C₃-alkyl group;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p, result in forming a 11-membered ring independently from the selection of variable A1, or A2;
B is independently selected from a —N(R¹⁵)— group and —O—;
G is an 1,2-arylene group or
   a monocyclic heteroarylene group having 5 or 6 ring atoms which contains at least one heteroatom and wherein two vicinal carbon atoms thereof are each bound to one of the adjacent alkylene groups, and which each are unsubstituted;
R⁸ is selected from a hydrogen atom, a methyl group, and a —CH₂—CH₂—(N-morpholino) group;
R⁹ is selected from a hydrogen atom and a C₁-C₃-alkyl group;
R¹⁵ is a hydrogen atom,
   a C₁-C₃-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected form a halogen atom, a C₁-C₃-alkyl group and/or a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I): in which
wherein
A is

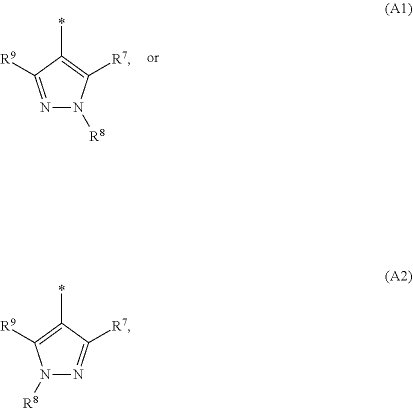

wherein R⁶ and R⁷, together with two carbon atoms of the pyrazole ring, two carbon atoms of the indole moiety and the nitrogen atom to which R⁶ is attached, form a 9- to 13-membered ring and * is the point of attachment of these moieties to the indole carbon atom bearing the A substituent;
R¹ and R² are each independently selected from a hydrogen atom, a halogen atom, a cyano group, a C₁-C₃-alkyl group, and a C₁-C₃-alkoxy group;
R³ is selected from a hydrogen atom, a halogen atom, a cyano group, a C₁-C₃-alkyl group, a C₁-C₃-haloalkyl group, a C₁-C₃-alkoxy group and a C₃-C₅-cycloalkyl group;
R⁴ is selected from an aryl group and a heteroaryl group, each of which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group and a C₃-C₅-cycloalkyl group;
L is a group —(CH₂)ₘ-E- wherein any CH₂ group is unsubstituted or substituted with one or two substituents and each substituent is independently selected from a halogen atom, a cyano group, a hydroxyl group, a C₁-C₃-alkyl group and a C₁-C₃-alkoxy group;
E is a bond, an oxygen atom, a sulfur atom, or a —NR¹⁴— group and constitutes the connecting element to R⁴,
m is 2, 3, or 4;
R⁵ is selected from a COOH group and a

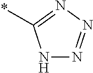 [SMILES: *c1nnn[nH]1]

group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, and a C₁-C₃-alkoxy group, wherein ^{#} is the point of attachment with the indole nitrogen atom and ^{##} is the point of attachment with the pyrazole carbon atom bearing the R⁷ substituent;
n is 1, 2, or 3;
t is 1;
r is 1, 2, or 3;
p is 1, 2, or 3;
where the integers selected for variables n, t, r, and p result in forming a 9- to 13-membered ring independently from the selection of variable A1 or A2;
B is independently selected from a —C(O)NR¹⁵— group, a —NR¹⁵C(O)— group, a —N(R¹⁵)— group, and —O—;
G is a 1,2-arylene group or
   a mono- or bicyclic heteroarylene group wherein two vicinal carbon atoms thereof are each bound to one of the adjacent alkylene groups,
   which are unsubstituted or substituted with one or more substituents independently selected from a halogen atom, a cyano group, a C₁-C₃-alkyl group, and a C₁-C₃-alkoxy group;
R⁸ is selected from a hydrogen atom, and
   a C₁-C₆-alkyl group, which is unsubstituted or substituted with one or more substituents independently selected from
      a halogen atom, a hydroxy group, a C₁-C₃-alkoxy group, a C₁-C₃-haloalkoxy group, a C₃-C₆-cycloalkyl group, a heterocycloalkyl group, and a NR²¹R²² group,
R⁹ is selected from a hydrogen atom,
   a C₁-C₄-alkyl group,
   a C₁-C₃-hydroxyalkyl group,
   a C₁-C₄-haloalkyl group,
   a C₁-C₄-haloalkyl-NH—C(O)—O—(C₁-C₃-alkylene)- group,
   a C₂-C₆-haloalkenyl group,
   a C₁-C₆-alkyl-O— group,
   a C₁-C₄-haloalkoxy group,
   a C₁-C₆-alkyl-O—(C₁-C₃-alkylene)- group,
   a (C₃-C₇)-cycloalkyl group,
   a (C₃-C₇)-cycloalkyl-O—(C₁-C₃-alkylene)- group,
   a phenyl-O—(C₁-C₃-alkylene)- group,
   a phenyl-(C₁-C₃-alkylene)-O—(C₁-C₃-alkylene)- group,
   a R¹⁹-(phenylene)-(C₁-C₃-alkylene)-O—(C₁-C₃-alkylene)- group,
   a R¹⁹-(phenylene)-O—(C₁-C₃-alkylene)- group,
   a R¹⁹-(phenylene)-(heteroarylene)-O—(C₁-C₃-alkylene)- group,
   a (R¹⁹)-(heterocycloalkylene)-(C₁-C₃-alkylene)- group,
   a (R¹⁹)-(heterocycloalkylene)-(phenylene)-O—(C₁-C₃-alkylene)- group,
   a (heterocycloalkenyl)-(phenylene)-O—(C₁-C₃-alkylene)- group,
   a (R¹⁹)-(heteroarylene)-(C₁-C₃-alkylene)-O—(C₁-C₃-alkylene)- group,
   a (R¹⁹)-(heteroarylene)-(phenylene)-O—(C₁-C₃-alkylene)- group,
   a (R²⁰)—S(O)₂-(phenylene)-O—(C₁-C₃-alkylene)- group,
   a (R²⁰)—S(O)₂—NH-(phenylene)-O—(C₁-C₃-alkylene)- group,
   a (R²⁰)—S(O)₂—NH-(phenylene)-O—(C₁-C₃-alkylene)- group,
   a (R²⁰)—S(O)₂—N(C₁-C₆-alkyl)-(phenylene)-O—(C₁-C₃-alkylene)- group,
   a (R²⁰)—S(O)₂-(heterocycloalkylene)-(phenylene)-O—(C₁-C₃-alkylene)- group,
   a (R¹⁹)-(heterocycloalkylene)-(heteroarylene)-O—(C₁-C₃-alkylene)- group,
   a (R²⁰)-(heteroarylene)-O—(C₁-C₃-alkylene)- group,
   a (R¹⁹)-(heteroarylene)-O—(C₁-C₃-alkylene)- group,
   a (R²⁰)—S(O)₂-(heteroarylene)-O—(C₁-C₃-alkylene)- group,
   a (R²⁰)—S(O)₂-(heterocycloalkylene)-(heteroarylene)-O—(C₁-C₃-alkylene)- group,
   a NR²¹R²²—(C₁-C₃-alkylene)- group,
   a (C₁-C₃-alkyl)-NH—(C₁-C₃-alkylene)- group,
   a (C₁-C₃-haloalkyl)-(C₁-C₃-alkylene)-NH—(C₁-C₃-alkylene)- group,
   a (C₁-C₃-haloalkyl)-NH—(C₁-C₃-alkylene)- group,
   a (C₁-C₃-alkyl)-NH—C(O)—(C₁-C₃-alkylene)- group,
   a (C₁-C₃-alkyl)-NR¹⁵—C(O)—(C₁-C₃-alkylene)- group,
   a (C₁-C₃-alkyl)-C(O)—NH—(C₁-C₃-alkylene)- group,
   a (C₁-C₃-alkyl)-C(O)—NR¹⁵—(C₁-C₃-alkylene)- group,
   a

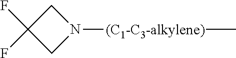

group, and a

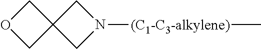

group,
where the phenyl ring is unsubstituted or substituted with a halogen atom, a hydroxyl group, or a C₁-C₃-alkoxy group and
the heterocycloalkyl group is unsubstituted or substituted with an oxo (═O) group or is unsubstituted or substituted with one or more substituents independently selected from a halogen atom, a hydroxyl group, a C₁-C₃-alkyl group, and a C₁-C₃-alkoxy group,
R¹⁴ is a hydrogen atom or a C₁-C₃-alkyl group;
R¹⁵ is independently selected from a hydrogen atom,
   a C₁-C₆-alkyl group
      which is unsubstituted or substituted with one or more substituents selected from a C₁-C₃-alkyl group, a heterocycloalkyl group, and an aryl group;
   a C₁-C₃-alkylene-C(O)— group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)—,
   an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group,
   a heterocycloalkyl-heteroarylene-S(O)₂— group,
   a phenyl group,
   a group

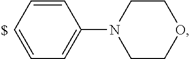

a group

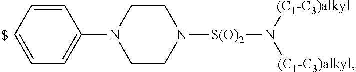

and
a group

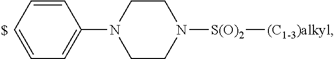

where $ is the point of attachment to the nitrogen atom, to which R¹⁵ is attached,
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
R²⁰ is selected from a C₁-C₃-alkyl group, a C₃-C₆-cycloalkyl group, and a NR²¹R²² group; and R²¹ and R²² are independently selected from a hydrogen atom and a C₁-C₆-alkyl group;
or a tautomer, an N-oxide, or a salt thereof, or a salt of a tautomer, or a salt of an N-oxide, or a mixture of same.

In accordance with a further aspect, the present invention provides compounds of general formula (I): in which
wherein
A is

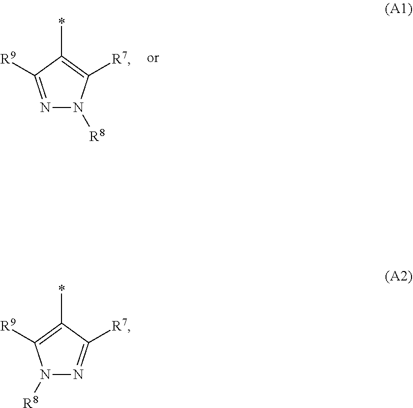

wherein R⁶ and R⁷, together with two carbon atoms of the pyrazole ring, two carbon atoms of the indole moiety and the nitrogen atom to which R⁶ is attached, form a 10- to 12-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three, substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 2, 3, or 4;
R⁵ is a COOH group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, and a C₁-C₃-alkoxy group, wherein ^{#} is the point of attachment with the indole nitrogen atom and ^{##} is the point of attachment with the pyrazole carbon atom bearing the R⁷ substituent;
n is 1 or 2;
t is 1;
r is 1 or 2;
p is 1 or 2;
where the integers selected for variables n, t, r, and p result in forming a 10- to 12-membered ring independently from the selection of variable A1 or A2;
B is selected from a —N(R¹⁵)— group and —O—,
G is a 1,2-arylene group or
   a mono- or bicyclic heteroarylene group wherein two vicinal carbon atoms thereof are each bound to one of the adjacent alkylene groups,
   which are unsubstituted or substituted with one or more substituents independently selected from a halogen atom, a cyano group, a C₁-C₃-alkyl group, and a C₁-C₃-alkoxy group;
R⁸ is selected from a hydrogen atom, and
   a C₁-C₆-alkyl group, which is unsubstituted or substituted with one or more substituents independently selected from
      a halogen atom, a hydroxy group, a C₃-C₆-cycloalkyl group and a heterocycloalkyl group;
R⁹ is selected from a hydrogen atom,
   a C₁-C₄-alkyl group,
   a C₁-C₃-hydroxyalkyl group,
   a C₁-C₄-haloalkyl group,
   a C₁-C₆-alkyl-O— group,
   a C₁-C₄-haloalkoxy group,
   a C₁-C₆-alkyl-O—(C₁-C₃-alkylene)- group,
   a (C₃-C₇)-cycloalkyl group,
   a R¹⁹-(phenylene)-O—(C₁-C₃-alkylene)- group,
   a NR²¹R²²—(C₁-C₃-alkylene)- group, and
   a (C₁-C₃-alkyl)-NH—(C₁-C₃-alkylene)- group;
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group, and
   a heterocycloalkyl-heteroarylene-S(O)₂— group;
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)-group, and a C₃-C₆-cycloalkyl-C(O)— group;
and
R²¹ and R²² are independently selected from a hydrogen atom and a C₁-C₆-alkyl group;
or a tautomer, an N-oxide, or a salt thereof or a salt of a tautomer or a salt of an N-oxide or a mixture of same.

In accordance with a further aspect, the present invention provides compounds of general formula (I): in which
wherein
A is

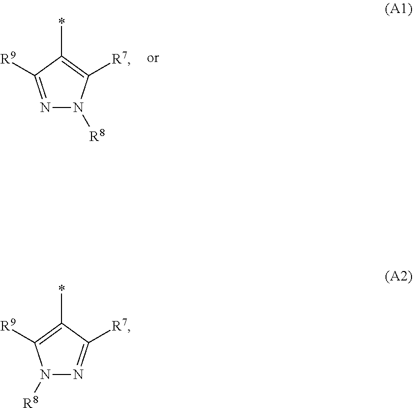

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 3;
R⁵ is a COOH group;
—R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is a 1,2-arylene group or
   a mono- or bicyclic heteroarylene group wherein two vicinal carbon atoms thereof are each bound to one of the adjacent alkylene groups,
   which are each independently unsubstituted or substituted with one or more substituents selected from a halogen atom and a C₁-C₃-alkyl group;
R⁸ is selected from a hydrogen atom, and
   a C₁-C₆-alkyl group, which is unsubstituted or substituted with a heterocycloalkyl group;
R⁹ is selected from a hydrogen atom,
   a C₁-C₄-alkyl group,
   a C₁-C₆-alkyl-O—(C₁-C₃-alkylene)- group,
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected from a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I): in which
A is

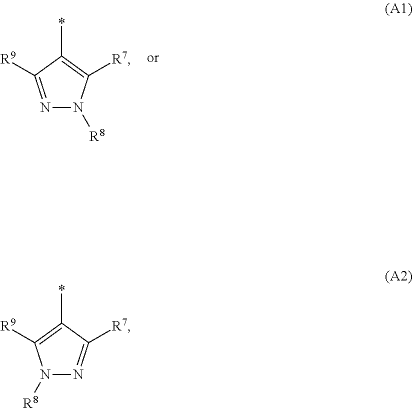

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from an aryl group and a heteroaryl group, each of which is unsubstituted or substituted with one, two, or three, substituents and each substituent is independently selected from a halogen atom and a C₁-C₃-alkyl group;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p, result in forming a 11-membered ring independently from the selection of variable A1, or A2;
B is independently selected from a —N(R¹⁵)— group and —O—;
G is an 1,2-arylene group or
   a mono- or bicyclic heteroarylene group whereby two vicinal carbon atoms thereof are each bound to one of the adjacent alkylene groups, and which each are unsubstituted;
R⁸ is selected from a hydrogen atom, a methyl group, and a —CH₂—CH₂—(N-morpholino) group;
R⁹ is selected from a hydrogen atom and a C₁-C₃-alkyl group;
R¹⁵ is a hydrogen atom,
   a C₁-C₃-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected form a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I):
wherein
A is

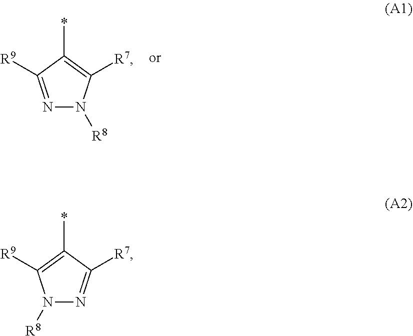

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from an aryl group and a heteroaryl group, each of which is unsubstituted or substituted with one, two, or three, substituents and each substituent is independently selected from a halogen atom and a C₁-C₃-alkyl group;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is an 1,2-arylene group or
   a mono- or bicyclic heteroarylene group wherein two vicinal carbon atoms thereof are each bound to one of the adjacent alkylene groups, and each are unsubstituted;
R⁸ is selected from a hydrogen atom, a methyl group, and a —CH₂—CH₂—(N-morpholino) group;
R⁹ is selected from a hydrogen atom and a C₁-C₃-alkyl group;
R¹⁵ is a hydrogen atom,
   a C₁-C₃-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected form a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I):
wherein
A is

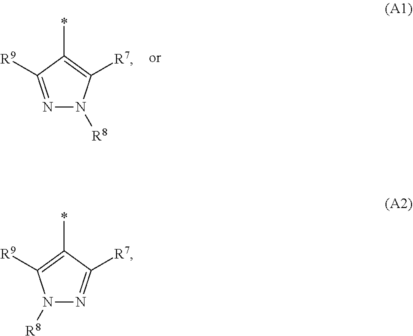

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from naphthalinyl, 6-fluoro-naphthalin-1-yl, 4-fluoro-naphthalin-1-yl, (5,6,7,8-tetrahydronaphthalen-1-yl), 2,3-dihydro-1H-inden-4-yl and 4-chloro-3,5-dimethylphen-1-yl;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1, or A2;
B is independently selected from a —N(R¹⁵)— group and —O—;
G is a *-(1,2-phenylene)-** group, a *-(2,3-quinoxalinylene)-** group, a *-(2,3-pyridinylene)-** group, a **-(2,3-pyridinylene)-* group and a *-(2,3-pyrazinylene)-** group, wherein the two vicinal carbon atoms thereof are each bound to one of the adjacent alkylene groups and * is the point of attachment to the —(CH₂)ₙ— group and the ** is the point of attachment to the —(CH₂)ᵣ— group;
R⁸ is selected from a hydrogen atom, a methyl group, and a —CH₂—CH₂—(N-morpholino) group;
R⁹ is selected from a hydrogen atom, a methyl group, and an ethyl group;
R¹⁵ is independently selected from a hydrogen atom, a methyl group, a (3,4,5-trimethoxybenzyl)carbamoyl group, a 6-(morpholin-4-yl)pyridin-3-yl]sulfonyl group, a tetrahydro-2H-pyran-4-yl-acetyl group, a tetrahydro-2H-pyran-4-ylcarbamoyl group, a tetrahydro-2H-pyran-4-ylmethyl)carbamoyl group, and a 2-(morpholin-4-yl)ethyl]sulfonyl group;
or a tautomer, an N-oxide, or a salt thereof, or a salt of a tautomer, or a salt of an N-oxide, or a mixture of same.

In accordance with a further aspect, the present invention provides compounds of general formula (I) wherein
A is

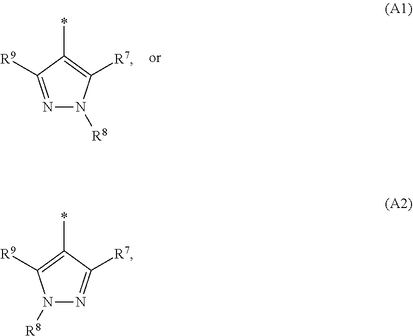

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 3;
R⁵ is a COOH group;
—R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is a 1,2-arylene group or
   a monocyclic heteroarylene group having 5 or 6 ring atoms which contains at least one heteroatom and wherein two vicinal carbon atoms thereof are each bound to one of the adjacent alkylene groups,
   which are each independently unsubstituted or substituted with one or more substituents selected from a halogen atom and a C₁-C₃-alkyl group;
R⁸ is selected from a hydrogen atom, and
   a C₁-C₆-alkyl group, which is unsubstituted or substituted with a heterocycloalkyl group;
R⁹ is selected from a hydrogen atom,
   a C₁-C₄-alkyl group,
   a C₁-C₆-alkyl-O—(C₁-C₃-alkylene)- group,
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected from a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I) wherein
A is

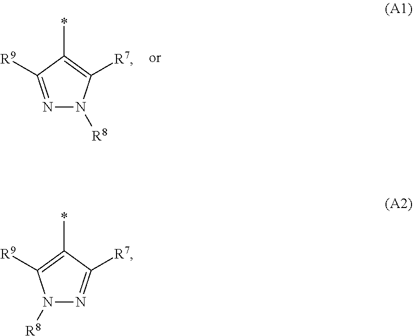

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 3;
R⁵ is a COOH group;
—R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is a 1,2-arylene group or
   a monocyclic heteroarylene group having 5 ring atoms which contains at least one heteroatom and wherein two vicinal carbon atoms thereof are each bound to one of the adjacent alkylene groups,
   which are each independently unsubstituted or substituted with one or more substituents selected from a halogen atom and a C₁-C₃-alkyl group;
R⁸ is selected from a hydrogen atom, and
   a C₁-C₆-alkyl group, which is unsubstituted or substituted with a heterocycloalkyl group;
R⁹ is selected from a hydrogen atom,
   a C₁-C₄-alkyl group,
   a C₁-C₆-alkyl-O—(C₁-C₃-alkylene)- group,
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected from a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In accordance with a further aspect, the present invention provides compounds of general formula (I) wherein
A is

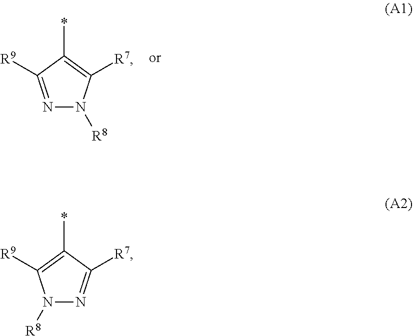

wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three substituents and each substituent is independently selected from a halogen atom, and a C₁-C₃-alkyl group;
L is a group —(CH₂)ₘ-E-;
E is a bond or oxygen atom and constitutes the connecting element to R⁴;
m is 3;
R⁵ is a COOH group;
—R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is a 1,2-arylene group or
   a monocyclic heteroarylene group having 6 ring atoms which contains at least one heteroatom and wherein two vicinal carbon atoms thereof are each bound to one of the adjacent alkylene groups,
   which are each independently unsubstituted or substituted with one or more substituents selected from a halogen atom and a C₁-C₃-alkyl group;
R⁸ is selected from a hydrogen atom, and
   a C₁-C₆-alkyl group, which is unsubstituted or substituted with a heterocycloalkyl group;
R⁹ is selected from a hydrogen atom,
   a C₁-C₄-alkyl group,
   a C₁-C₆-alkyl-O—(C₁-C₃-alkylene)- group,
R¹⁵ is independently selected from a hydrogen atom,
   a C₁-C₆-alkyl group,
   a heterocycloalkyl-(C₁-C₃-alkylene)-C(O)— group,
   a heterocycloalkyl-(C₁-C₃-alkylene)-S(O)₂— group,
   a heterocycloalkyl-NH—C(O)— group,
   a heterocycloalkyl-(C₁-C₃-alkylene)-NH—C(O)— group,
   a heterocycloalkyl-heteroarylene-S(O)₂— group, and
   an aryl-(C₁-C₃-alkylene)-NH—C(O)— group, which unsubstituted or substituted with 1, 2, or 3 substituents independently selected from a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

In some embodiments, the present invention includes compounds of general formula (I) selected from:
(rac)-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid,
4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid-N-ethylethanamine salt (enantiomer 1),
4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid,
4,5-dimethyl-17-[3-(naphthalen-1-yloxy)propyl]-5,7,9,14-tetrahydropyrazolo[4′,3′:9,10]-pyrido[3′,2′:3,4][1,6]oxazacycloundecino[8,7,6-hi]indole-16-carboxylic acid (enantiomer 1),
4,5-dimethyl-17-[3-(naphthalen-1-yloxy)propyl]-5,7,9,14-tetrahydropyrazolo[4′,3′:9,10]-pyrido[3′,2′:3,4][1,6]oxazacycloundecino[8,7,6-hi]indole-16-carboxylic acid (enantiomer 2),
(rac)-4,5-dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid,
4,5-dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid-N-ethylethanamine salt (enantiomer 1),
4,5-dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid-N-ethylethanamine salt (enantiomer 2),
(rac)-4,5-dimethyl-19-[3-(naphthalen-1-yloxy)propyl]-5,7,9,16-tetrahydroindolo[1′,7′:6,7,8]-pyrazolo[4′,3′:9,10][1,6]oxazacycloundecino[3,4-b]quinoxaline-18-carboxylic acid,
(rac)-4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-7,8,9,14-tetrahydro-5H-indolo[1,7-bc]pyrazolo-[4,3-e][2,8]benzodiazacycloundecine-16-carboxylic acid trifluoroacetate salt (enantiomer 1),
4,5-dimethyl-17-[3-(naphthalen-1-yloxy)propyl]-7,8,9,14-tetrahydro-5H-indolo[1,7-bc]pyrazolo-[4,3-e][2,8]benzodiazacycloundecine-16-carboxylic acid trifluoroacetate salt (enantiomer 2),
(rac)-4,5,8-trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid,
4,5,8-trimethyl-17-[3-(naphthalen-1-yloxy)propyl]-7,8,9,14-tetrahydro-5H-indolo[1,7-bc]-pyrazolo[4,3-e][2,8]benzodiazacycloundecine-16-carboxylic acid-N-ethylethanamine salt (enantiomer 1),
4,5,8-trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid acetate salt,
4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid (enantiomer 1),
4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-17-[3-(naphthalen-1-yloxy)propyl]-8-[(3,4,5-trimethoxybenzyl)carbamoyl]-7,8,9,14-tetrahydro-5H-indolo[1,7-bc]pyrazolo[4,3-e][2,8]benzodiazacycloundecine-16-carboxylic acid,
(rac)-4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic acid (enantiomer 1),
4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic acid (enantiomer 1),
4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-17-[3-(naphthalen-1-yloxy)propyl]-8-[(tetrahydro-2H-pyran-4-ylmethyl)-carbamoyl]-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylic acid (enantiomer 1),
4,5-dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-8-{[2-(morpholin-4-yl)ethyl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid,
4,5-dimethyl-8-[2-(morpholin-4-yl)ethanesulfonyl]-17-{3-[(naphthalen-1-yl)oxy]propyl}-7,8,9,14-tetrahydro-5H-indolo[1,7-bc]pyrazolo[4,3-e][2,8]benzodiazacycloundecine-16-carboxylic acid-N-ethylethanamine salt (enantiomer 1),
4,5-dimethyl-8-[2-(morpholin-4-yl)ethanesulfonyl]-17-{3-[(naphthalen-1-yl)oxy]propyl}-7,8,9,14-tetrahydro-5H-indolo[1,7-bc]pyrazolo[4,3-e][2,8]benzodiazacycloundecine-16-carboxylic acid-N-ethylethanamine salt (enantiomer 2),
(rac)-3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid,
3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid-N-ethylethanamine salt (enantiomer 1),
3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid-N-ethylethanamine salt (enantiomer 2),
(rac)-3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid,
3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid (enantiomer 1),
3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid (enantiomer 2),
(rac)-3-chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid,
3-chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid-N-ethylethanamine salt (enantiomer 1),
3-chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid-N-ethylethanamine salt (enantiomer 2),
(rac)-3-chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid,
3-chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid-N-ethylethanamine salt (enantiomer 1),
3-chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid-N-ethylethanamine salt (enantiomer 2),
(rac)-3-chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic acid,
3-chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic acid (enantiomer 1),
3-chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic acid (enantiomer 2),
(rac)-3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid,
3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 1),
3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic acid,
3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic acid (enantiomer 1),
3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic acid (enantiomer 2),
(rac)-3-chloro-4-ethyl-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic acid,
3-chloro-4-ethyl-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic acid N-ethylethanamine salt (enantiomer 1),
3-chloro-4-ethyl-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic acid N-ethylethanamine salt (enantiomer 2),
(rac)-3-chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid,
3-chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid-N-ethylethanamine salt (enantiomer 1),
3-chloro-4-ethyl-17-{3-[(6-fluoronaphthalen-1-yl)oxy]propyl}-6-methyl-6,7,9,14-tetrahydroindolo[7,1-fg]pyrazolo[3,4-d][2,8]benzoxazacycloundecine-16-carboxylic acid-N-ethylethanamine salt (enantiomer 2),
(rac)-1-(3-(4-chloro-3,5-dimethylphenoxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylic acid,
(rac)-1-(3-((2,3-dihydro-1H-inden-4-yl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylic acid,
(rac)-15-fluoro-13,14-dimethyl-1-(3-((5,6,7,8-tetrahydronaphthalen-1-yl)oxy)propyl)-4,9,11,13-tetrahydrobenzo[3,4]pyrazolo[4′,3′:9,10][1,6]oxaazacycloundecino[8,7,6-hi]indole-2-carboxylic acid,
(rac)-15-fluoro-1-(3-((4-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylic acid,
(rac)-15-fluoro-1-(3-((6-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylic acid,
3-fluoro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 1),
3-fluoro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-15-fluoro-13,14-dimethyl-1-(3-(naphthalen-1-yloxy)propyl)-4,9,11,13-tetrahydrobenzo[3,4]pyrazolo[4′,3′:9,10][1,6]oxaazacycloundecino[8,7,6-hi]indole-2-carboxylic acid,
3-fluoro-4,5-dimethyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 1),
3-fluoro-4,5-dimethyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-14-ethyl-15-fluoro-13-methyl-1-(3-(naphthalen-1-yloxy)propyl)-4,9,11,13-tetrahydrobenzo[3,4]pyrazolo[4′,3′:9,10][1]oxa[6]azacycloundecino[8,7,6-hi]indole-2-carboxylic acid,
4-ethyl-3-fluoro-5-methyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 1),
4-ethyl-3-fluoro-5-methyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-14-ethyl-15-fluoro-1-(3-((6-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylic acid,
4-ethyl-3-fluoro-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic acid-N-ethylethanamine salt (enantiomer 1),
4-ethyl-3-fluoro-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic acid-N-ethylethan-amine salt (enantiomer 2),
(rac)-14-ethyl-15-fluoro-1-(3-((4-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylic acid,
(rac)-14-ethyl-15-fluoro-13-methyl-1-(3-((5,6,7,8-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylic acid,
(rac)-1-(3-((2,3-dihydro-1H-inden-4-yl)oxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylic acid,
(rac)-1-(3-(4-chloro-3,5-dimethylphenoxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylic acid,
(rac)-15-fluoro-12,14-dimethyl-1-(3-(naphthalen-1-yloxy)propyl)-4,9,11,12-tetrahydrobenzo-[3,4]pyrazolo[4′,3′:9,10][1]oxa[6]azacycloundecino[8,7,6-hi]indole-2-carboxylic acid,
(rac)-15-fluoro-1-(3-((6-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-15-fluoro-1-(3-((4-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-15-fluoro-12,14-dimethyl-1-(3-((5,6,7,8-tetrahydronaphthalen-1-yl)oxy)propyl)-4,9,11,12-tetrahydrobenzo[3,4]pyrazolo[4′,3′:9,10][1]oxa[6]azacycloundecino[8,7,6-hi]indole-2-carboxylic acid,
(rac)-1-(3-((2,3-dihydro-1H-inden-4-yl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-1-(3-(4-chloro-3,5-dimethylphenoxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-1-(3-(4-chloro-3,5-dimethylphenoxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-1-(3-((2,3-dihydro-1H-inden-4-yl)oxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-12-methyl-1-(3-((5,6,7,8-tetrahydronaphthalen-1-yl)oxy)propyl)-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-1-(3-((4-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-1-(3-((6-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-12-methyl-1-(3-(naphthalen-1-yloxy)propyl)-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-13-methyl-1-(3-((1,2,3,4-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylic acid,
(rac)-3-chloro-17-{3-[(6-fluoronaphthalen-1-yl)oxy]propyl}-4,5-dimethyl-5,7,9,14-tetrahydropyrazino[2′,3′:3,4]pyrazolo[4′,3′:9,10][1,6]oxazacycloundecino[8,7,6-hi]indole-16-carboxylic acid and
(rac)-3-chloro-4,5-dimethyl-16-[3-(1-naphthyloxy)propyl]-5,7-dihydro-9H,13H-[1,2]oxazolo[3′,4′:3,4]pyrazolo[4′,3′:9,10][1,6]oxazacycloundecino[8,7,6-hi]indole-15-carboxylic acid
or a tautomer, an N-oxide, or a salt thereof, or a salt of a tautomer or a salt of an N-oxide, or a mixture of same.

In some embodiments, the present invention includes compounds of general formula (I) selected from example 1 to example 93, or a tautomer, an N-oxide, or a salt thereof, or a salt of a tautomer, or a salt of an N-oxide, or a mixture of same.

In one embodiment the invention includes the compound of example 94.

In some embodiments, the present invention includes compounds of formula (I), supra, in which R¹, R², and R³ are each selected from a hydrogen atom, a halogen atom, and a C₁-C₃-alkyl group, or a tautomer, an N-oxide, or a salt thereof, or a salt of a tautomer, or a salt of an N-oxide, or a mixture of same.

In some embodiments, the present invention includes compounds of formula (I), supra, in which R¹, R², and R³ are each selected from a hydrogen atom, a halogen atom, a C₁-C₃-alkyl group and a C₁-C₃-alkoxy group, or a tautomer, an N-oxide, or a salt thereof, or a salt of a tautomer, or a salt of an N-oxide, or a mixture of same.

In some embodiments, the present invention includes compounds of formula (I), supra, in which R¹, R², and R³ are each selected from a hydrogen atom, a fluorine atom or a chlorine atom and a C₁-C₃-alkyl group, or a tautomer, an N-oxide, or a salt thereof or a salt of a tautomer or a salt of an N-oxide or a mixture of same.

In some embodiments, the present invention includes compounds of formula (I), supra, in which R¹, R², and R³ are each selected from a hydrogen atom, a fluorine atom or a chlorine atom or a tautomer, an N-oxide, or a salt thereof, or a salt of a tautomer, or a salt of an N-oxide, or a mixture of same.

In some embodiments, the present invention includes compounds of formula (I), supra, in which R¹ and R² are each independently selected from a hydrogen atom, a halogen atom, a cyano group, and a C₁-C₃-alkyl group or a tautomer, an N-oxide, or a salt thereof, or a salt of a tautomer, or a salt of an N-oxide, or a mixture of same.

In some embodiments, the present invention includes compounds of formula (I), supra, in which R¹ and R² are a hydrogen atom or a tautomer, an N-oxide, or a salt thereof, or a salt of a tautomer, or a salt of an N-oxide, or a mixture of same.

In some embodiments, the present invention includes compounds of formula (I), supra, in which R¹ is selected from a hydrogen atom, a halogen atom and a C₁-C₃-alkyl group or a tautomer, an N-oxide, or a salt thereof, or a salt of a tautomer, or a salt of an N-oxide, or a mixture of same.

In some embodiments, the present invention includes compounds of formula (I), supra, in which R¹ is a hydrogen atom or a fluorine atom or a tautomer, an N-oxide, or a salt thereof, or a salt of a tautomer, or a salt of an N-oxide, or a mixture of same.

In some embodiments, the present invention includes compounds of formula (I), supra, in which R² is a hydrogen atom or a tautomer, an N-oxide, or a salt thereof, or a salt of a tautomer, or a salt of an N-oxide, or a mixture of same.

In some embodiments, the present invention includes compounds of formula (I), supra, in which R¹ is a hydrogen atom or a fluorine atom and R² is a hydrogen atom and R³ is a hydrogen atom or a tautomer, an N-oxide, or a salt thereof, or a salt of a tautomer, or a salt of an N-oxide, or a mixture of same.

In some embodiments, the present invention includes compounds of formula (I), supra, in which R³ is selected from a hydrogen atom, a halogen atom, a cyano group, a C₁-C₃-alkyl group, a C₁-C₃-haloalkyl group, a C₁-C₃-alkoxy group, a C₁-C₃-alkylthio group, a —S(O)—(C₁-C₃-alkyl) group, a —S(O)₂—(C₁-C₃-alkyl) group, a C₁-C₃-haloalkoxy group, a C₁-C₃-haloalkylthio group, and a C₃-C₅-cycloalkyl group or a tautomer, an N-oxide, or a salt thereof, or a salt of a tautomer, or a salt of an N-oxide, or a mixture of same.

In some embodiments, the present invention includes compounds of formula (I), supra, in which R³ is selected from a hydrogen atom, a halogen atom, a cyano group, a C₁-C₃-alkyl group, a C₁-C₃-haloalkyl group, a C₁-C₃-alkoxy group or a tautomer, an N-oxide, or a salt thereof, or a salt of a tautomer, or a salt of an N-oxide, or a mixture of same.

In some embodiments, the present invention includes compounds of formula (I), supra, in which R³ is a hydrogen atom.

In some embodiments, the present invention includes compounds of formula (I), supra, in which R⁴ is selected from an aryl group and a heteroaryl group, which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group, a C₁-C₃-thioalkyl group, a C₁-C₃-haloalkoxy group, a (C₁-C₃)-haloalkyl-S— group, and a C₃-C₅-cycloalkyl group, or a tautomer, an N-oxide, or a salt thereof, or a salt of a tautomer, or a salt of an N-oxide, or a mixture of same.

In some embodiments, the present invention includes compounds of formula (I), supra, in which R⁴ is an aryl group, which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group, a C₁-C₃-thioalkyl group, a C₁-C₃-haloalkoxy group, a (C₁-C₃)-haloalkyl-S— group, and a C₃-C₅-cycloalkyl group, or a tautomer, an N-oxide, or a salt thereof, or a salt of a tautomer, or a salt of an N-oxide, or a mixture of same.

In some embodiments, the present invention includes compounds of formula (I), supra, in which R⁴ is an aryl group, which is unsubstituted or substituted with one, two, or three substituents and each substituent is independently selected from a halogen atom, a C₁-C₃-alkyl group, and a C₁-C₃-haloalkyl group, or a tautomer, an N-oxide, or a salt thereof, or a salt of a tautomer, or a salt of an N-oxide, or a mixture of same.

In some embodiments, the present invention includes compounds of formula (I), supra, in which R⁴ is an aryl group, which is unsubstituted or substituted with one, two, or three substituents and each substituent is independently selected from a halogen atom and a C₁-C₃-alkyl group, or a tautomer, an N-oxide, or a salt thereof, or a salt of a tautomer, or a salt of an N-oxide, or a mixture of same.

In some embodiments, the present invention includes compounds of formula (I), supra, in which R⁴ is an naphthyl group, which is unsubstituted or substituted with one, two, or three substituents and each substituent is independently selected from a halogen atom and a C₁-C₃-alkyl group, or a tautomer, an N-oxide, or a salt thereof, or a salt of a tautomer, or a salt of an N-oxide, or a mixture of same.

In some embodiments, the present invention includes compounds of formula (I), supra, in which R⁴ is an naphthyl group, which is unsubstituted or substituted with a fluorine atom or a chlorine atom, or a tautomer, an N-oxide, or a salt thereof or a salt of a tautomer or a N-oxide or a mixture of same.

In other embodiments, the present invention includes compounds of formula (I), supra, in which R⁴ is a heteroaryl group, which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group, a C₁-C₃-thioalkyl group, a C₁-C₃-haloalkoxy group, and a C₁-C₃-halothioalkyl group, or a tautomer, an N-oxide, or a salt thereof, or a salt of a tautomer, or a salt of an N-oxide, or a mixture of same.

In other embodiments, the present invention includes compounds of formula (I), supra, in which R⁴ is a heteroaryl group, which is unsubstituted or substituted with one, two, three, four or five substituents and each substituent is independently selected from a halogen atom, a cyano group, a C₁-C₃-alkyl group, a C₁-C₃-haloalkyl group, and a C₁-C₃-alkoxy group, or a tautomer, an N-oxide, or a salt thereof, or a salt of a tautomer, or a salt of an N-oxide, or a mixture of same.

In further embodiments the present invention includes compounds of formula (I), supra, in which R⁴ is selected from a 1-naphthyl group, a 4-chloro-3,5-dimethyl-phenyl-1-yl group, and a 5,6,7,8-tetrahydronaphthalene-1-yl group, or a tautomer, an N-oxide, or a salt thereof, or a salt of a tautomer, or a salt of an N-oxide, or a mixture of same.

In further embodiments the present invention includes compounds of formula (I), supra, in which R⁴ is a 1-naphthyl group, which is unsubstituted or substituted one or two times with a group selected from a fluorine atom, a chlorine atom, a methyl group or a trifluoromethyl group. or a tautomer, an N-oxide, or a salt thereof, or a salt of a tautomer, or a salt of an N-oxide, or a mixture of same.

In other embodiments, the present invention includes compounds of formula (I), supra, in which R⁴ is selected from a 6-fluoro-naphthalin-1-yl group, a 4-fluoro-naphthalin-1-yl group, a (5,6,7,8-tetrahydronaphthalen-1-yl) group, a 2,3-dihydro-1H-inden-4-yl group, a 4-chloro-3,5-dimethylphen-1-yl group, each of which are unsubstituted or substituted with one, two, three, four or five substituents, particularly one, two or three substituents, and each substituent is independently selected from a halogen atom, a C₁-C₃-alkyl group, a C₁-C₃-haloalkyl group, a C₁-C₃-alkoxy group, and a C₁-C₃-haloalkoxy group, or a tautomer, an N-oxide, or a salt thereof, or a salt of a tautomer, or a salt of an N-oxide, or a mixture of same.

In other embodiments, the present invention includes compounds of formula (I), supra, in which R⁴ is selected from a 6-fluoro-naphthalin-1-yl group, a 4-fluoro-naphthalin-1-yl group, a (5,6,7,8-tetrahydronaphthalen-1-yl) group, a 2,3-dihydro-1H-inden-4-yl group, a 4-chloro-3,5-dimethylphen-1-yl group, each of which are unsubstituted or substituted with one, two or three substituents, and each substituent is independently selected from a halogen atom, a C₁-C₃-alkyl group, a C₁-C₃-haloalkyl group, a C₁-C₃-alkoxy group, and a C₁-C₃-haloalkoxy group, or a tautomer, an N-oxide, or a salt thereof, or a salt of a tautomer, or a salt of an N-oxide, or a mixture of same.

In other embodiments, the present invention includes compounds of formula (I), supra, in which R⁴ is selected from a phenyl group, a naphthyl group, and a 5,6,7,8-tetrahydronaphthyl group, each of which are unsubstituted or substituted with one substituent, which is selected from a halogen atom and a C₁-C₃-alkyl group, or a tautomer, an N-oxide, or a salt thereof, or a salt of a tautomer, or a salt of an N-oxide, or a mixture of same.

In other embodiments, the present invention includes compounds of formula (I), supra, in which R⁴ is a 4-chloro-3,5-dimethyl-phenyl-1-yl group, or a tautomer, an N-oxide, or a salt thereof, or a salt of a tautomer, or a salt of an N-oxide, or a mixture of same.

In further embodiments, the present invention includes compounds of formula (I), supra, in which R⁴ is a 5,6,7,8-tetrahydronaphthalene-1-yl group, or a tautomer, an N-oxide, or a salt thereof, or a salt of a tautomer, or a salt of an N-oxide, or a mixture of same.

In further embodiments, the present invention includes compounds of formula (I), supra, in which R⁴ is a 1-naphthyl group, or a tautomer, an N-oxide, or a salt thereof, or a salt of a tautomer, or a salt of an N-oxide, or a mixture of same.

In further embodiments, the present invention includes compounds of formula (I), supra, in which R⁴ is a 1-naphthyl group, which is unsubstituted or substituted one or two times with a group selected from a fluorine atom, a chlorine atom, a methyl group and a trifluoromethyl group, or a tautomer, an N-oxide, or a salt thereof, or a salt of a tautomer, or a salt of an N-oxide, or a mixture of same.

In some embodiments, the present invention includes compounds of formula (I), supra, in which R⁴ is a naphthyl group, which is unsubstituted or substituted with a fluorine atom or a chlorine atom or tautomer, an N-oxide, or a salt thereof or a salt of a tautomer or a salt of an N-oxide or a mixture of same.

In some embodiments, the present invention includes compounds of formula (I), supra, in which R⁴ is a naphthyl group, which is unsubstituted or substituted with a fluorine atom or a tautomer, an N-oxide, or a salt thereof, or a salt of a tautomer, or a salt of an N-oxide, or a mixture of same.

In some embodiments, the present invention includes compounds of formula (I), supra, in which R⁴ is a naphthyl group, which is unsubstituted or substituted with a chlorine atom or a tautomer, an N-oxide, or a salt thereof, or a salt of a tautomer, or a salt of an N-oxide, or a mixture of same.

In some embodiments, the present invention includes compounds of formula (I), supra, in which R⁴ is a naphth-1-yl group or a tautomer, an N-oxide, or a salt thereof, or a salt of a tautomer, or a salt of an N-oxide, or a mixture of same.

In further embodiments, the present invention includes compounds of formula (I), supra, in which R⁴ is a halo-naphthyl group, particularly 6-halo-naphthyl group, more particularly a 6-chloro-naphthaline-1-yl group or a 6-fluoro-naphthaline-1-yl group or a tautomer, an N-oxide, or a salt thereof, or a salt of a tautomer, or a salt of an N-oxide, or a mixture of same.

In further embodiments, the present invention includes compounds of formula (I), supra, in which R⁴ is selected from a 4-chloro-3,5-dimethyl-phenyl-1-yl group and a 5,6,7,8-tetrahydronaphthalene-1-yl group, or a tautomer, an N-oxide, or a salt thereof, or a salt of a tautomer, or a salt of an N-oxide, or a mixture of same.

In other embodiments, the present invention includes compounds of formula (I), supra, in which R⁴ has one, two or three optional substituents, more particularly one substituent.

In other embodiments, the present invention includes compounds of formula (I), supra, in which L is a group —(CH₂)ₘ-E- which is unsubstituted or substituted with one or two substituents and each substituent is independently selected from a halogen atom, a hydroxyl group, a C₁-C₃-alkyl group, and a C₁-C₃-alkoxy group, or two substituents are taken together with their intervening atoms to form a saturated or partially unsaturated 3-6-membered cycloalkyl ring, or a 3-8 membered saturated or partially unsaturated heterocyclic ring having 1-2 heteroatoms independently selected from an oxygen atom, a sulfur atom, a —S(O)— group, a —S(O)₂— group, and a —NR¹⁴— group, or a tautomer, an N-oxide, or a salt thereof, or a salt of a tautomer, or a salt of an N-oxide, or a mixture of same.

In other embodiments, the present invention includes compounds of formula (I), supra, in which L is a group —(CH₂)ₘ-E- which is unsubstituted or substituted with one or two substituents and each substituent is independently selected from a halogen atom, a hydroxyl group, a C₁-C₃-alkyl group, and a C₁-C₃-alkoxy group, or two substituents are taken together with their intervening atoms to form a saturated or partially unsaturated 3-6-membered ring, or a 3-8 membered saturated or partially unsaturated heterocyclic ring having 1-2 heteroatoms independently selected from an oxygen atom, a sulfur atom, a —S(O)— group, a —S(O)₂— group, and a —NR¹⁴— group, or a tautomer, an N-oxide, or a salt thereof, or a salt of a tautomer, or a salt of an N-oxide, or a mixture of same.

In other embodiments, the present invention includes compounds of formula (I), supra, in which L is a —(CH₂)ₘ-E- group which is unsubstituted or substituted with a C₁-C₃-alkyl group, particularly with a methyl group or a tautomer, an N-oxide, or a salt thereof, or a salt of a tautomer, or a salt of an N-oxide, or a mixture of same.

In other embodiments, the present invention includes compounds of formula (I), supra, in which L is an unsubstituted group —(CH₂)ₘ-E- or a tautomer, an N-oxide, or a salt thereof, or a salt of a tautomer, or a salt of an N-oxide, or a mixture of same.

In further embodiments the present invention includes compounds of formula (I), supra, in which E is an oxygen atom or a tautomer, an N-oxide, or a salt thereof, or a salt of a tautomer, or a salt of an N-oxide, or a mixture of same.

In other embodiments, the present invention includes compounds of formula (I), supra, in which L is a —(CH₂)ₘ-E- group which is unsubstituted or substituted with a C₁-C₃-alkyl group, particularly with a methyl group, and E is an oxygen atom or a tautomer, an N-oxide, or a salt thereof, or a salt of a tautomer, or a salt of an N-oxide, or a mixture of same.

In other embodiments, the present invention includes compounds of formula (I), supra, in which L is an unsubstituted group —(CH₂)ₘ-E- and E is an oxygen atom or a tautomer, an N-oxide, or a salt thereof, or a salt of a tautomer, or a salt of an N-oxide, or a mixture of same.

In yet further embodiments, the present invention includes compounds of formula (I), supra, in which R⁵ is a COOH group, a

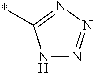 [SMILES: *c1nnn[nH]1]

group, a —C(O)—NHS(O)₂(C₁-C₆-alkyl) group, a —C(O)—NHS(O)₂(C₃-C₆-cycloalkyl) group, a —C(O)—NHS(O)₂(aryl) group, a —C(O)—NHS(O)₂(CH₂)₂NHCO(C₁-C₆-alkyl) group, a —C(O)—NHS(O)₂(CH₂)₂NHCO(C₃-C₆-cycloalkyl) group, or a —C(O)—NHS(O)₂(CH₂)₂NHCO(aryl) group, or a tautomer, an N-oxide, or a salt thereof, or a salt of a tautomer, or a salt of an N-oxide, or a mixture of same.

In further embodiments, the present invention includes compounds of formula (I), supra, in R⁵ is a COOH group, or a

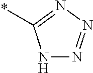 [SMILES: *c1nnn[nH]1]

group, or a tautomer, an N-oxide, or a salt thereof, or a salt of a tautomer, or a salt of an N-oxide, or a mixture of same.

In further embodiments, the present invention includes compounds of formula (I), supra, in R⁵ is a COOH group, or a tautomer, an N-oxide, or a salt thereof, or a salt of a tautomer, or a salt of an N-oxide, or a mixture of same.

In other embodiments, the present invention includes compounds of formula (I), supra, in which —R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent; or a tautomer, an N-oxide, or a salt thereof, or a salt of a tautomer, or a salt of an N-oxide, or a mixture of same.

In other embodiments, the present invention includes compounds of formula (I), supra, in which —R⁶-R¹⁰— is ^{#}—(CH₂)ₙ-(G)-(CH₂)ᵣ—(B)ₜ—(CH₂)ₚ—^{##}, where one or more CH₂ groups are unsubstituted or substituted with one or more substituents selected from a halogen atom, a hydroxyl group, a NR¹⁷R¹⁸ group, a C₁-C₃-alkyl group, a C₁-C₃-haloalkyl group, a C₁-C₃-alkoxy group, and a C₁-C₃-haloalkoxy group and a (heterocycloalkyl)-(C₁-C₃-alkylene)- group, wherein ^{#} is the point of attachment with the indole nitrogen atom and ^{##} is the point of attachment with the carbon atom of the phenyl moiety bearing the R¹⁰ substituent; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a mono- or bicyclic heteroarylene group wherein two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a mono- or bicyclic heteroarylene group wherein two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a mono- or bicyclic heteroarylene group whereby two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group
or a monocyclic heteroarylene group whereby two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group
or a monocyclic heteroarylene group having 5 or 6 ring atoms which contains at least one heteroatom whereby two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 5 ring atoms which contains at least one heteroatom whereby two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 6 ring atoms which contains at least one heteroatom whereby two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group; or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a mono- or bicyclic heteroarylene group whereby two vicinal carbon atoms thereof are each bound to one of the adjacent alkylene groups,
which are each independently unsubstituted or substituted with one or more substituents selected from a halogen atom and a C₁-C₃-alkyl group;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group whereby two vicinal carbon atoms thereof are each bound to one of the adjacent alkylene groups,
which are each independently unsubstituted or substituted with one or more substituents selected from a halogen atom and a C₁-C₃-alkyl group;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 5 or 6 ring atoms which contains at least one heteroatom whereby two vicinal carbon atoms thereof are each bound to one of the adjacent alkylene groups,
which are each independently unsubstituted or substituted with one or more substituents selected from a halogen atom and a C₁-C₃-alkyl group;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 5 ring atoms which contains at least one heteroatom whereby two vicinal carbon atoms thereof are each bound to one of the adjacent alkylene groups,
which are each independently unsubstituted or substituted with one or more substituents selected from a halogen atom and a C₁-C₃-alkyl group;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 6 ring atoms which contains at least one heteroatom whereby two vicinal carbon atoms thereof are each bound to one of the adjacent alkylene groups,
which are each independently unsubstituted or substituted with one or more substituents selected from a halogen atom and a C₁-C₃-alkyl group;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a mono- or bicyclic heteroarylene group whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 5 or 6 ring atoms which contains at least one heteroatom whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 5 ring atoms which contains at least one heteroatom whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 5 ring atoms which contains two heteroatoms whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 6 ring atoms which contains at least one heteroatom whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 6 ring atoms which contains two heteroatoms whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is an 1,2-arylene group or
a monocyclic heteroarylene group having 6 ring atoms which contains two nitrogen atoms whereby two vicinal carbon atoms thereof are each bound to one of the adjacent alkylene groups, which each are unsubstituted;
or a tautomer, an N-oxide, or a salt thereof, or a salt of a tautomer, or a salt of an N-oxide, or a mixture of same.

In other embodiments, the present invention includes compounds of formula (I), supra, in which G is a *-(1,2-phenylene)-** group, a *-(2,3-quinoxalinylene)-** group, a *-(2,3-pyridinylene)-** group, a **-(2,3-pyridinylene)-* group and a *-(2,3-pyrazinylene)-**group, wherein the two vicinal carbon atoms thereof are each bound to one of the adjacent alkylene groups and * is the point of attachment to the —(CH₂)ₙ group and the ** is the point of attachment to the —(CH₂)ₛ— group; or a tautomer, an N-oxide, or a salt thereof, or a salt of a tautomer, or a salt of an N-oxide, or a mixture of same.

In other embodiments, the present invention includes compounds of formula (I), supra, in which G is a *-(1,2-phenylene)-** group, a *-(2,3-quinoxalinylene)-** group, a *-(2,3-pyridinylene)-** group, a **-(2,3-pyridinylene)-* group, a *-(2,3-pyrazinylene)-**group and a **-(2,3-oxazolyl)-* group, wherein the two vicinal carbon atoms thereof are each bound to one of the adjacent alkylene groups and * is the point of attachment to the —(CH₂)ₙ group and the ** is the point of attachment to the —(CH₂)ₛ— group; or a tautomer, an N-oxide, or a salt thereof, or a salt of a tautomer, or a salt of an N-oxide, or a mixture of same.

In further embodiments, the present invention includes compounds of formula (I), supra, in which B is independently selected from a —C(O)NR¹⁵— group, a —NR¹⁵C(O)— group, a —N(R¹⁵)— group, a —N(R¹⁵)—C(═O)—N(R¹⁵)— group, a —O—C(═O)—N(R¹⁵)— group, a —N(R¹⁵)—C(═O)—O— group, —O—, —S—, —S(O)—, —S(O)₂—, a —S(O)NR¹⁵— group, a —NR¹⁵S(O)— group, a —S(O)₂NR¹⁵— group, a —NR¹⁵S(O)₂— group, or a tautomer, an N-oxide, or a salt thereof, or a salt of a tautomer, or a salt of an N-oxide, or a mixture of same.

In further embodiments, the present invention includes compounds of formula (I), supra, in which B is independently selected from a —C(O)NR¹⁵— group, a —NR¹⁵C(O)— group, a —N(R¹⁵)— group, —O—, —S—, —S(O)— and —S(O)₂—, or a tautomer, an N-oxide, or a salt thereof, or a salt of a tautomer, or a salt of an N-oxide, or a mixture of same.

In further embodiments, the present invention includes compounds of formula (I), supra, in which B is independently selected from a —N(R¹⁵)— group, —O—, —S—, —S(O)— and —S(O)₂—, or a tautomer, an N-oxide, or a salt thereof, or a salt of a tautomer, or a salt of an N-oxide, or a mixture of same.

In further embodiments, the present invention includes compounds of formula (I), supra, in which B is independently selected from a —C(O)NR¹⁵— group, a —NR¹⁵C(O)— group, a —N(R¹⁵)— group, —O— and —S— or a tautomer, an N-oxide, or a salt thereof, or a salt of a tautomer, or a salt of an N-oxide, or a mixture of same.

In further embodiments, the present invention includes compounds of formula (I), supra, in which B is independently selected from a —C(O)NR¹⁵— group, a —NR¹⁵C(O)— group, a —N(R¹⁵)— group, and —O— or a tautomer, an N-oxide, or a salt thereof, or a salt of a tautomer, or a salt of an N-oxide, or a mixture of same.

In further embodiments, the present invention includes compounds of formula (I), supra, in which B is independently selected from —O—, —S— and a —N(R¹⁵)— group or a tautomer, an N-oxide, or a salt thereof, or a salt of a tautomer, or a salt of an N-oxide, or a mixture of same.

In further embodiments, the present invention includes compounds of formula (I), supra, in which B is an oxygen atom or —NR¹⁵— or a tautomer, an N-oxide, or a salt thereof, or a salt of a tautomer, or a salt of an N-oxide, or a mixture of same.

In further embodiments, the present invention includes compounds of formula (I), supra, in which B is an oxygen atom or a tautomer, an N-oxide, or a salt thereof, or a salt of a tautomer, or a salt of an N-oxide, or a mixture of same.

In further embodiments, the present invention includes compounds of formula (I), supra, in which B is —NR¹⁵—, and R¹⁵ particularly is a hydrogen atom, a C₁-C₆-alkyl group, a heterocycloalkyl-(C₁-C₃-alkylene)-C(O)— group, a heterocycloalkyl-(C₁-C₃-alkylene)-S(O)₂— group, a heterocycloalkyl-NH—C(O)— group, an aryl-(C₁-C₃-alkylene)-NH—C(O)— group, which unsubstituted or substituted with 1, 2, or 3 substituents independently selected form a halogen atom a C₁-C₃-alkyl group or a C₁-C₃-alkoxy group, more particularly a hydrogen atom, a methyl group, a (3,4,5-trimethoxybenzyl)carbamoyl group, a 6-(morpholin-4-yl)pyridin-3-yl]sulfonyl group, a tetrahydro-2H-pyran-4-yl-acetyl group, a tetrahydro-2H-pyran-4-ylcarbamoyl group, a tetrahydro-2H-pyran-4-ylmethyl)carbamoyl group, 2-(morpholin-4-yl)ethyl]sulfonyl group or a tautomer, an N-oxide, or a salt thereof, or a salt of a tautomer, or a salt of an N-oxide, or a mixture of same.

In further embodiments, the present invention includes compounds of formula (I), supra, in which B is a cation with R¹⁶ as the respective anion, particularly

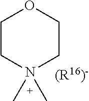

or —N⁺(R²¹R²²)(R¹⁶)⁻, more particularly

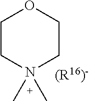

or —N⁺(CH)₂(R¹⁶)⁻ or a tautomer, an N-oxide, or a salt thereof, or a salt of a tautomer, or a salt of an N-oxide, or a mixture of same.

In other embodiments, the present invention includes compounds of formula (I), supra, in which A is A1, or a tautomer, an N-oxide, or a salt thereof, or a salt of a tautomer, or a salt of an N-oxide, or a mixture of same.

In further embodiments, the present invention includes compounds of formula (I), supra, in which A is A2, or a tautomer, an N-oxide, or a salt thereof, or a salt of a tautomer, or a salt of an N-oxide, or a mixture of same.

In yet other embodiments, the present invention includes compounds of formula (I), supra, in which A is A3, or a tautomer, an N-oxide, or a salt thereof, or a salt of a tautomer, or a salt of an N-oxide, or a mixture of same.

In yet other embodiments, the present invention includes compounds of formula (I), supra, in which A is A3 and the macrocyclic ring is a 9-membered-, a 10-membered-, a 11-membered-, a 12-membered-, a 13-membered-, a 14-membered-, a 15-membered- or a 16-membered ring, particularly a 9-membered-, a 10-membered-, a 11-membered-, or a 12-membered ring, more particularly a 12-membered ring or a tautomer, an N-oxide, or a salt thereof, or a salt of a tautomer, or a salt of an N-oxide, or a mixture of same.

In yet other embodiments, the present invention includes compounds of formula (I), supra, in which A is A3 whereby unsubstituted or one or two of the groups selected from CR¹¹, CR¹² or CR¹³ may be replaced by a nitrogen atom, wherein R⁶ and R¹⁰, together with three carbon atoms of the phenyl ring, two carbon atoms of the indole moiety and the nitrogen atom to which R⁶ is attached, form a 9- to 16-membered ring and * is the point of attachment of these moieties to the indole carbon atom bearing the A substituent; or a tautomer, an N-oxide, or a salt thereof, or a salt of a tautomer, or a salt of an N-oxide, or a mixture of same.

In yet other embodiments, the present invention includes compounds of formula (I), supra, in which A is A3 wherein R⁶ and R¹⁰, together with three carbon atoms of the phenyl ring, two carbon atoms of the indole moiety and the nitrogen atom to which R⁶ is attached, form a 9- to 16-membered ring and * is the point of attachment of these moieties to the indole carbon atom bearing the A substituent; or a tautomer, an N-oxide, or a salt thereof, or a salt of a tautomer, or a salt of an N-oxide, or a mixture of same.

In yet other embodiments, the present invention includes compounds of formula (I), supra, in which A is A3 wherein R⁶ and R¹⁰, together with three carbon atoms of the phenyl ring, two carbon atoms of the indole moiety and the nitrogen atom to which R⁶ is attached, form a 11- to 16-membered ring and * is the point of attachment of these moieties to the indole carbon atom bearing the A substituent; or a tautomer, an N-oxide, or a salt thereof, or a salt of a tautomer, or a salt of an N-oxide, or a mixture of same.

In yet other embodiments, the present invention includes compounds of formula (I), supra, in which A is A3 wherein R⁶ and R¹⁰, together with three carbon atoms of the phenyl ring, two carbon atoms of the indole moiety and the nitrogen atom to which R⁶ is attached, form a 12-membered ring and * is the point of attachment of these moieties to the indole carbon atom bearing the A substituent; or a tautomer, an N-oxide, or a salt thereof, or a salt of a tautomer, or a salt of an N-oxide, or a mixture of same.

In further embodiments, the present invention includes compounds of formula (I), supra, in which A is A1 or A2, or a tautomer, an N-oxide, or a salt thereof, or a salt of a tautomer, or a salt of an N-oxide, or a mixture of same.

In other embodiments, the present invention includes compounds of formula (I), supra, in which A is A1 or A2 and the macrocyclic ring is a 9-membered-, a 10-membered-, a 11-membered-, a 12-membered-, a 13-membered-, a 14-membered-, a 15-membered- or a 16-membered ring, particularly a 9- to 12-membered ring or a 12- or a 13-membered ring, more particularly a 10- to 11-membered ring, or a tautomer, an N-oxide, or a salt thereof, or a salt of a tautomer, or a salt of an N-oxide, or a mixture of same.

In other embodiments, the present invention includes compounds of formula (I), supra, in which A is A1 or A2, which together with the indole moiety and the R⁶-R⁷ form a 9-membered-, a 10-membered-, a 11-membered- or a 12-membered macrocyclic ring or a tautomer, an N-oxide, or a salt thereof, or a salt of a tautomer, or a salt of an N-oxide, or a mixture of same.

In yet other embodiments, the present invention includes compounds of formula (I), supra, in which A is A1, which together with the indole moiety and the R⁶-R⁷ form a 9-membered-, a 10-membered-, a 11-membered- or a 12-membered macrocyclic ring or a tautomer, an N-oxide, or a salt thereof, or a salt of a tautomer, or a salt of an N-oxide, or a mixture of same.

In yet other embodiments, the present invention includes compounds of formula (I), supra, in which A is A2, which together with the indole moiety and the R⁶-R⁷ form a 9-membered-, a 10-membered-, a 11-membered- or a 12-membered macrocyclic ring or a tautomer, an N-oxide, or a salt thereof, or a salt of a tautomer, or a salt of an N-oxide, or a mixture of same.

In yet other embodiments, the present invention includes compounds of formula (I), supra, in which A is A1 or A2, which together with the indole moiety and the R⁶-R⁷ form a 10-membered-, a 11-membered- or a 12-membered macrocyclic ring or a tautomer, an N-oxide, or a salt thereof, or a salt of a tautomer, or a salt of an N-oxide, or a mixture of same.

In yet other embodiments, the present invention includes compounds of formula (I), supra, in which A is A1 or A2, which together with the indole moiety and the R⁶-R⁷ form a 11-membered macrocyclic ring or a tautomer, an N-oxide, or a salt thereof, or a salt of a tautomer, or a salt of an N-oxide, or a mixture of same.

In yet other embodiments, the present invention includes compounds of formula (I), supra, in which A is A1 and R⁸ and R⁹ are C₁-C₃-alkyl or a tautomer, an N-oxide, or a salt thereof, or a salt of a tautomer, or a salt of an N-oxide, or a mixture of same.

In still other embodiments, the present invention includes compounds of formula (I), supra, in which A is A2 and R⁸ and R⁹ are independently selected from C₁-C₃-alkyl, particularly from methyl or ethyl or a tautomer, an N-oxide, or a salt thereof, or a salt of a tautomer, or a salt of an N-oxide, or a mixture of same.

In further embodiments, the present invention includes compounds of formula (I), supra, in which A is A1 or A2 and R⁸ and R⁹ are independently selected from C₁-C₃-alkyl or a —(C₁-C₃-alkyl)-heterocycloalkyl group or a tautomer, an N-oxide, or a salt thereof, or a salt of a tautomer, or a salt of an N-oxide, or a mixture of same.

The integers selected for variables n, t, p, q, r, s and v may result in different ring sizes but still the rings obtained have to fulfill the rule that only rings of a ring size of 9 members up to a ring size of 16 members are encompassed.

In further embodiments, the present invention includes compounds of formula (I), supra, in which
n is 1, 2, 3, 4, 5, 6, 7, 8, 9, or 10;
t is 0 or 1;
p is 0, 1, 2, 3, 4, 5, or 6;
r is 0, 1, 2, or 3;
s is 0, 1, 2, or 3;
or a tautomer, an N-oxide, or a salt thereof, or a salt of a tautomer, or a salt of an N-oxide, or a mixture of same.

In further embodiments, the present invention includes compounds of formula (I), supra, in which
n is 1, 2, or 3;
t is 1;
p is 1, 2, or 3;
r is 1, 2, or 3;
or a tautomer, an N-oxide, or a salt thereof, or a salt of a tautomer, or a salt of an N-oxide, or a mixture of same.

In further embodiments, the present invention includes compounds of formula (I), supra, in which
n is 1, 2, or 3;
t is 1;
p is 1;
r is 1, 2, or 3;
or a tautomer, an N-oxide, or a salt thereof, or a salt of a tautomer, or a salt of an N-oxide, or a mixture of same.

In further embodiments, the present invention includes compounds of formula (I), supra, in which n, t, p and r are 1.

The limitations relating to A1 and A2 are independent from the limitations relating to A3.

In further embodiments, the present invention includes compounds of formula (I), supra, in which R⁸ is selected from
a C₁-C₆-alkyl group, which is unsubstituted or substituted with one or more substituents independently selected from
   a halogen atom, a hydroxyl group, a C₁-C₃-alkoxy group, a C₁-C₃-haloalkoxy group, a C₃-C₆-cycloalkyl group, a heterocycloalkyl group, and a NR²¹R²² group; or
a C₁-C₃-haloalkyl group,
a C₃-C₆-cycloalkyl group and
a C₁-C₆-alkyl group in which one or two not directly adjacent carbon atoms are independently replaced by a hetero atom selected from —O— and —NH—, or a tautomer, an N-oxide, or a salt thereof or a salt of a tautomer or a N-oxide or a mixture of same.

In further embodiments, the present invention includes compounds of formula (I), supra, in which R⁸ is selected from
a C₁-C₃-alkyl group, which is unsubstituted or substituted with one or more substituents independently selected from
   a halogen atom, a hydroxyl group, a C₁-C₃-alkoxy group, a C₁-C₃-haloalkoxy group, a C₃-C₆-cycloalkyl group, a heterocycloalkyl group, and a NR²¹R²² group; or
or a tautomer, an N-oxide, or a salt thereof, or a salt of a tautomer, or a salt of an N-oxide, or a mixture of same.

In further embodiments, the present invention includes compounds of formula (I), supra, in which R⁸ is a C₁-C₆-alkyl group which is unsubstituted or substituted with a C₃-C₆-cycloalkyl group or a heterocycloalkyl group or a tautomer, an N-oxide, or a salt thereof, or a salt of a tautomer, or a salt of an N-oxide, or a mixture of same.

In further embodiments, the present invention includes compounds of formula (I), supra, in which R⁸ is a C₁-C₆-alkyl group which is unsubstituted or substituted with a heterocycloalkyl group or a tautomer, an N-oxide, or a salt thereof, or a salt of a tautomer, or a salt of an N-oxide, or a mixture of same.

In further embodiments, the present invention includes compounds of formula (I), supra, in which R⁸ is selected from methyl, ethyl, 1,1,1-trifluoroethyl, and morpholino-ethyl, or a tautomer, an N-oxide, or a salt thereof, or a salt of a tautomer, or a salt of an N-oxide, or a mixture of same.

In further embodiments, the present invention includes compounds of formula (I), supra, in which R⁸ is selected from methyl and morpholino-ethyl, or a tautomer, an N-oxide, or a tautomer, an N-oxide, or a salt thereof, or a salt of a tautomer, or a salt of an N-oxide, or a mixture of same.

In further embodiments, the present invention includes compounds of formula (I), supra, in which R⁹ is a hydrogen atom,
a C₁-C₄-alkyl group,
a C₁-C₃-hydroxyalkyl group,
a C₁-C₄-haloalkyl group,
a C₁-C₄-haloalkyl-NH—C(O)—O—(C₁-C₃-alkylene)- group,
a C₂-C₆-haloalkenyl group,
a C₁-C₆-alkyl-O-group,
a C₁-C₄-haloalkoxy group,
a C₁-C₆-alkyl-O—(C₁-C₃-alkylene)- group,
a (C₃-C₇)-cycloalkyl group,
a (C₃-C₇)-cycloalkyl-O—(C₁-C₃-alkylene)- group,
a phenyl-O—(C₁-C₃-alkylene)- group,
a phenyl-(C₁-C₃-alkylene)-O—(C₁-C₃-alkylene)- group,
a R¹⁹-phenyl-(C₁-C₃-alkylene)-O—(C₁-C₃-alkylene)- group,
a R¹⁹-phenylene-O—(C₁-C₃-alkylene)- group,
a R¹⁹-phenyl-heteroaryl-O—(C₁-C₃-alkylene) group,
a (R¹⁹)-(heterocycloalkyl)-(C₁-C₃-alkylene)- group,
a (R¹⁹)-(heterocycloalkylene)-(phenylene)-O—(C₁-C₃-alkylene)- group,
a (heterocycloalkenylene)-(phenylene)-O—(C₁-C₃-alkylene)- group,
a (R¹⁹)-(heteroaryl-(C₁-C₃-alkylene)-O—(C₁-C₃-alkylene)- group,
a (R¹⁹)-(heteroaryl)-(phenylene)-O—(C₁-C₃-alkylene)- group,
a (R²⁰)—S(O)₂-(phenylene)-O—(C₁-C₃-alkylene)- group,
a (R²⁰)—S(O)₂—NH-(phenylene)-O—(C₁-C₃-alkylene)- group,
a (R²⁰)—S(O)₂—NH-(phenylene)-O—(C₁-C₃-alkylene)- group,
a (R²⁰)—S(O)₂—N(C₁-C₆-alkyl)-(phenylene)-O—(C₁-C₃-alkylene)- group,
a (R²⁰)—S(O)₂-(heterocycloalkylene)-(phenylene)-O—(C₁-C₃-alkylene)- group,
a (R¹⁹)-(heterocycloalkylene)-(heteroarylene)-O—(C₁-C₃-alkylen)- group,
a (R²⁰)-(heteroarylene)-O—(C₁-C₃-alkylene)- group,
a (R¹⁹)-(heteroarylene)-O—(C₁-C₃-alkylene)- group,
a (R²⁰)—S(O)₂-(heteroarylene)-O—(C₁-C₃-alkylene)- group,
a (R²⁰)—S(O)₂-(heterocycloalkylene)-(heteroarylene)-O—(C₁-C₃-alkylene)- group,
a NR²¹R²²—(C₁-C₃-alkylene)- group,
a (C₁-C₃-alkyl)-NH—(C₁-C₃-alkylene)- group,
a (C₁-C₃-haloalkyl)-(C₁-C₃-alkylene)-NH—(C₁-C₃-alkylene)- group,
a (C₁-C₃-haloalkyl)-NH—(C₁-C₃-alkylene)- group,
a (C₁-C₃-alkyl)-NH—C(O)—(C₁-C₃-alkylene)- group,
a (C₁-C₃-alkyl)-NR¹⁵—C(O)—(C₁-C₃-alkylene)- group,
a (C₁-C₃-alkyl)-C(O)—NH—(C₁-C₃-alkylene)- group,
a (C₁-C₃-alkyl)-C(O)—NR¹⁵—(C₁-C₃-alkylene)- group,
a

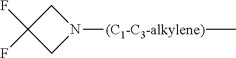

group or a

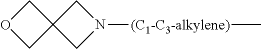

group, where the phenyl ring is unsubstituted or substituted with a halogen atom, a hydroxyl group, or a C₁-C₃-alkoxy group and
the heterocycloalkyl group is unsubstituted or substituted with an oxo (═O) group or is substituted with one or more substituents independently selected from a halogen atom, and a C₁-C₃-alkyl group, or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from hydrogen atom, a C₁-C₄-alkyl group, a C₁-C₃-hydroxyalkyl group, a C₁-C₄-haloalkyl group, a C₁-C₆-alkyl-O— group, a C₁-C₄-haloalkoxy group, a C₁-C₆-alkyl-O—(C₁-C₃-alkylene)- group, a (C₃-C₇)-cycloalkyl group, a R¹⁹-(phenylene)-O—(C₁-C₃-alkylene)- group, a NR²¹R²²—(C₁-C₃-alkylene)- group, a (C₁-C₃-alkyl)-NH—(C₁-C₃-alkylene)- group, or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from a C₁-C₄-alkyl group, a C₁-C₃-hydroxyalkyl group, a C₁-C₄-haloalkyl group, a C₁-C₆-alkyl-O— group, a C₁-C₄-haloalkoxy group, a C₁-C₆-alkyl-O—(C₁-C₃-alkylene)- group, a (C₃-C₇)-cycloalkyl group, a R¹⁹-(phenylene)-O—(C₁-C₃-alkylene)- group, a NR²¹R²²—(C₁-C₃-alkylene)- group, a (C₁-C₃-alkyl)-NH—(C₁-C₃-alkylene)- group, or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from a hydrogen atom, a C₁-C₄-alkyl group, a C₁-C₃-hydroxyalkyl group, a C₁-C₄-haloalkyl group, a C₁-C₆-alkyl-O— group, and a C₁-C₆-alkyl-O—(C₁-C₃-alkylene)- group, or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from a C₁-C₄-alkyl group, a C₁-C₃-hydroxyalkyl group, a C₁-C₄-haloalkyl group, a C₁-C₆-alkyl-O— group, and a C₁-C₆-alkyl-O—(C₁-C₃-alkylene)- group, or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from hydrogen atom, a C₁-C₄-alkyl group, and a C₁-C₆-alkyl-O—(C₁-C₃-alkylene)- group or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from a C₁-C₄-alkyl group, and a C₁-C₆-alkyl-O—(C₁-C₃-alkylene)- group or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from a methyl group, an ethyl group and a N-morpholinoethyl group or a tautomer, an N-oxide, or a salt thereof, or a salt of a tautomer, or a salt of an N-oxide, or a mixture of same.

In further embodiments, the present invention includes compounds of formula (I), supra, in which R⁹ is selected from a methyl group, and a N-morpholinoethyl group or a tautomer, an N-oxide, or a salt thereof, or a salt of a tautomer, or a salt of an N-oxide, or a mixture of same.

In further embodiments, the present invention includes compounds of formula (I), supra, in which R¹² is a methoxy group, or a tautomer, an N-oxide, or a salt thereof, or a salt of a tautomer, or a salt of an N-oxide, or a mixture of same.

In further embodiments, the present invention includes compounds of formula (I), supra, in which R¹² is hydrogen or a methoxy group, or a tautomer, an N-oxide, or a salt thereof, or a salt of a tautomer, or a salt of an N-oxide, or a mixture of same.

In further embodiments, the present invention includes compounds of formula (I), supra, in which R¹³ is hydrogen or a methyl group, or a tautomer, an N-oxide, or a salt thereof, or a salt of a tautomer, or a salt of an N-oxide, or a mixture of same.

In further embodiments, the present invention includes compounds of formula (I), supra, in which R¹⁵ is selected from a C₁-C₆-alkyl group
which is unsubstituted or substituted with one or more substituents selected from a halogen atom, a C₁-C₃-alkyl group, a C₁-C₃-haloalkyl group, a C₁-C₃-hydroxyalkyl group, a C₁-C₃-alkoxy group, a C₁-C₃-haloalkoxy group, a heterocycloalkyl group, an aryl group, a (R¹⁹)-(heterocycloalkylene)-(arylene)-O— group, a (heterocycloalkyl)-(arylene)-O— group, an aryl-O— group, an aryl-(C₁-C₃-alkylene)-O— group, a (R²⁰)—S(O)₂-arylene-O— group, a (R²⁰)S(O)₂-heterocycloalkylene-arylene-O— group, an aryl-heteroarylene-O— group, an aryl-heteroarylene-O—(C₁-C₃-alkylene)- group, a heterocycloalkyl-(C₁-C₃-alkylene)-C(O)— group, a heterocyclyl-NH—C(O) group, a aryl-(C₁-C₃-alkylene)-NH—C(O)— group, a heterocycloalkylene-(C₁-C₃-alkylene)-S(O)₂— group, and a heterocycloalkylene-heteroarylene-S(O)₂— group;
a C₁-C₃-alkylene-C(O)— group,
a heterocycloalkyl-(C₁-C₃-alkylene)-C(O)— group,
a heterocycloalkyl-(C₁-C₃-alkylene)-S(O)₂— group,
a heterocyclyl-NH—C(O)— group,
a heterocycloalkyl-(C₁-C₃-alkylene)-NH—C(O)—,
an aryl-(C₁-C₃-alkylene)-NH—C(O)— group, which unsubstituted or substituted with 1, 2, or 3 substituents independently selected form a halogen atom, a C₁-C₃-alkyl group or a C₁-C₃-alkoxy group,
a heterocycloalkyl-heteroarylene-S(O)₂— group,
a phenyl group,
a group

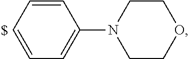

a group

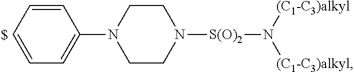

and
a group

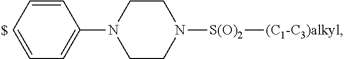

where $ is the point of attachment to the nitrogen atom, to which R¹⁵ is attached
or a tautomer, an N-oxide, or a salt thereof, or a salt of a tautomer, or a salt of an N-oxide, or a mixture of same.

In further embodiments, the present invention includes compounds of formula (I), supra, in which R¹⁵ is selected from
R¹⁵ is independently selected from a hydrogen atom, a C₁-C₆-alkyl group
   which is unsubstituted or substituted with one or more substituents selected from a C₁-C₃-alkyl group, a heterocycloalkyl group, and an aryl group;
a C₁-C₃-alkylene-C(O)— group,
a heterocycloalkyl-(C₁-C₃-alkylene)-C(O)— group,
a heterocycloalkyl-(C₁-C₃-alkylene)-S(O)₂— group,
a heterocyclyl-NH—C(O)— group,
a heterocycloalkyl-(C₁-C₃-alkylene)-NH—C(O)—,
an aryl-(C₁-C₃-alkylene)-NH—C(O)— group, which unsubstituted or substituted with 1, 2, or 3 substituents independently selected form a halogen atom, a C₁-C₃-alkyl group or a C₁-C₃-alkoxy group,
a heterocycloalkyl-heteroarylene-S(O)₂— group,
a phenyl group,
a group

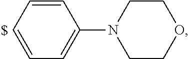

a group

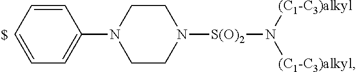

and
a group

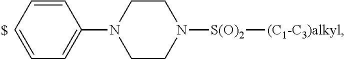

where $ is the point of attachment to the nitrogen atom, to which R¹⁵ is attached, or a tautomer, an N-oxide, or a salt thereof, or a salt of a tautomer, or a salt of an N-oxide, or a mixture of same.

In further embodiments, the present invention includes compounds of formula (I), supra, in which R¹⁵ is independently selected from a hydrogen atom, a C₁-C₆-alkyl group, a heterocycloalkyl-(C₁-C₃-alkylene)-C(O)— group, a heterocycloalkyl-(C₁-C₃-alkylene)-S(O)₂— group, a heterocycloalkyl-NH—C(O)— group, heterocycloalkyl-(C₁-C₃-alkylene)-NH—C(O)— group, a heterocycloalkyl-heteroarylene-S(O)₂— group, and
an aryl-(C₁-C₃-alkylene)-NH—C(O)— group, which is unsubstituted or substituted with 1, 2, or 3 substituents independently selected form a halogen atom a C₁-C₃-alkyl group or a C₁-C₃-alkoxy group, or a tautomer, an N-oxide, or a salt thereof, or a salt of a tautomer, or a salt of an N-oxide, or a mixture of same.

In further embodiments, the present invention includes compounds of formula (I), supra, in which R¹⁵ is independently selected from a hydrogen atom, a methyl group, a (3,4,5-trimethoxybenzyl)carbamoyl group, a 6-(morpholin-4-yl)pyridin-3-yl]sulfonyl group, a tetrahydro-2H-pyran-4-yl-acetyl group, a tetrahydro-2H-pyran-4-ylcarbamoyl group, a tetrahydro-2H-pyran-4-ylmethyl)carbamoyl group, 2-(morpholin-4-yl)ethyl]sulfonyl group, or a tautomer, an N-oxide, or a salt thereof, or a salt of a tautomer, or a salt of an N-oxide, or a mixture of same.

In further embodiments, the present invention includes compounds of formula (I), supra, in which R¹⁶ is selected from pharmaceutically acceptable anions, preferably selected from Cl⁻, Br⁻, Acetate (CH₃CO₂)⁻, trifluoroacetate (CF₃CO₂)⁻, and formate (HCO₂)⁻ or an inner salt of the anion of another portion of the same molecule, or where two molecule zwitter ions form two salt pairs or a tautomer, an N-oxide, or a salt thereof, or a salt of a tautomer, or a salt of an N-oxide, or a mixture of same.

R¹⁹ and R²⁰ are substituents which may be located at any position of the residue bearing such substituent which can be addressed by chemically suitable methods irrespective at which position such residue may bear further substituents, e.g. a morpholine ring bearing a R¹⁹ substituent can be 2-methyl-morpholine or 3-methyl-morpholine or a tetrahydropyrane bearing a R¹⁹ substituent can be 3-hydroxy-tetrahydropyrane or 4-hydroxy-tetrahydropyrane leading to e.g. R⁹ is a [4-(4-hydroxytetrahydro-2H-pyran-4-yl)phenoxy]methyl- group or a tautomer, an N-oxide, or a salt thereof, or a salt of a tautomer, or a salt of an N-oxide, or a mixture of same.

In further embodiments, the present invention includes compounds of formula (I), supra, wherein R¹⁹ is selected from a hydrogen atom, a hydroxyl group, a cyano group, a C₁-C₃-alkyl group, a C₁-C₆-hydroxyalkyl group, a C₁-C₃-alkoxy group, a C(O)OR²¹—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group; or a tautomer, an N-oxide, or a salt thereof, or a salt of a tautomer, or a salt of an N-oxide, or a mixture of same.

In yet further embodiments, the present invention includes compounds of formula (I), supra, in which R¹⁹ is a C₁-C₃-alkyl group, or a tautomer, an N-oxide, or a salt thereof, or a salt of a tautomer, or a salt of an N-oxide, or a mixture of same.

In further embodiments, the present invention includes compounds of formula (I), supra, wherein R²⁰ is selected from a C₁-C₃-alkyl group, a C₃-C₆-cycloalkyl group, and a NR²¹R²² group or a tautomer, an N-oxide, or a tautomer, an N-oxide, or a salt thereof, or a salt of a tautomer, or a salt of an N-oxide, or a mixture of same.

In further embodiments, the present invention includes compounds of formula (I), or a tautomer, an N-oxide, or a salt thereof, or a salt of a tautomer or an N-oxide, or a mixture of same.

In further embodiments, the present invention includes compounds of formula (I), or a salt thereof.

In further embodiments, the present invention includes compounds of formula (I), or a tautomer, or a salt thereof, or a salt of a tautomer, or a mixture of same.

In further embodiments, the present invention includes compounds of formula (I), which are salts.

In further embodiments, the present invention includes compounds of formula (I), which are a tautomer or a salt thereof, or a salt of a tautomer, or a mixture of same.

In further embodiments, the present invention includes compounds of formula (I), which are a an N-oxide, or a salt thereof, or a salt of an N-oxide, or a mixture of same.

In further embodiments of the first aspect, the present invention provides combinations of two or more of the above mentioned embodiments under the heading “further embodiments of the first aspect of the present invention”.

Furthermore it is understood that the invention includes any subcombination of the disclosed single embodiments herein for certain residues or subcombination of residues of formula (I).

The present invention includes any sub-combination within any embodiments or aspects of the present invention of compounds of general formula (I), supra.

The present invention includes any sub-combination within any embodiments or aspects of the present invention of compounds or intermediate compounds of general formula (I or II). The present invention includes the compounds of general formula (I) which are disclosed in the Example Section of this text, infra.

General Synthesis of Compounds of General Formula (I) of the Present Invention

A. General Synthesis Route

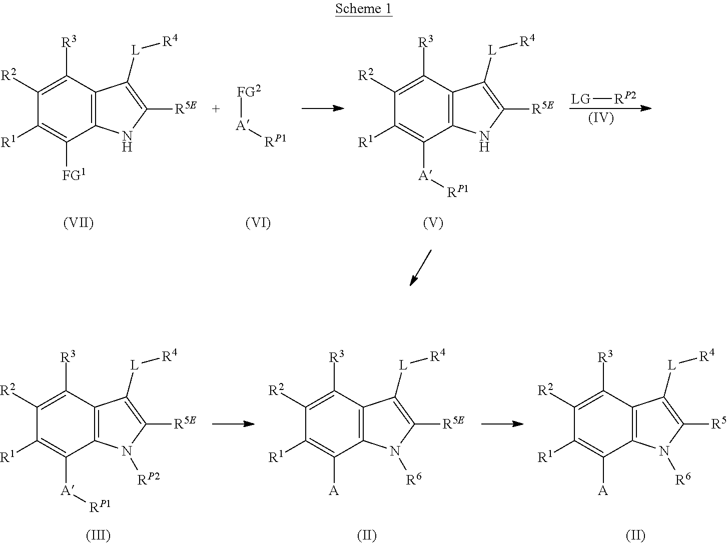

Compounds of general formula (I) can be synthesized according to the general synthesis route depicted in Scheme 1, encompassing a Suzuki coupling of starting materials of formulae (VII) and (VI) to give intermediates of formula (V), elaboration of the macrocyclic core by attachment of a group R^{p2} to the indole nitrogen present in compounds of formula (V), by reaction with compounds of formula (IV), in which LG represents a leaving group as defined herein and R^{p2} is discussed below, followed by (or together in one step with) macrocyclisation of the resulting intermediates of formula (III), e.g. by intramolecular nucleophilic substitution, to give macrocyclic intermediates of formula (II). Dependent inter alia on the nature of R^{p1} and R^{p2}, which together give rise to a group ^{#}—(CH₂)ₙ-(G)-(CH₂)ᵣ—(B)ₜ—(CH₂)ₚ—^{##} as defined for the compounds of general formula (I) after elaboration into the compounds of the present invention, the conversion of compounds of formula (V) into said macrocyclic intermediates of formula (II) may proceed with or without the intermediacy of intermediates of formula (III) e.g. directly from the compounds of formula (V) to the macrocyclic intermediates of formula (II) without requiring the use of compounds of formula (IV); for details see e.g. the Schemes 2a-2e, infra. Finally, conversion of R^{5E} into R⁵, e.g. by ester saponification, optionally followed by conversion of the resulting carboxylic acid into an acylsulfonamide according to methods known to the person skilled in the art (see for example: Bioorg. Med Chem. Lett. 2006, 16, 3639-3641; Bioorg. Med Chem. Lett. 2012, 22, 713-717; Org. Lett. 2012, 14(2), 556-559), yields the compounds of formula (I).

Said general synthesis route commences with a well-known Suzuki coupling of compounds of formula (VII), in which R¹, R², R³, R⁴ and L are as defined for the compounds of general formula (I), and in which R^{5E} represents a group suitable to act as a precursor of a —C(═O)OH or a tetrazol-5-yl group, preferably a group —C(═O)O—C₁₋₄-alkyl, with compounds of formula (VI), in which A′, together with the group R^{p1} attached to it, represents a group suitable to act as precursor of a group A as defined for the compounds of general formula (I), to give compounds of formula (V). The group R⁴, constituting the terminus of the side chain attached to C-3 of the indole core in formula (VII), can alternatively be established on later stage (see e.g. Scheme 2e and its discussion for details). Examples of groups A′ are exemplified further below in this chapter.

In formulae (VI) and (VII), FG¹ in combination with FG² represents a pair of functional groups together enabling a Suzuki coupling; either FG¹ represents chloro, bromo, iodo or a trifluoromethanesulfonyl- group, preferably bromo or iodo, and FG² represents a group —B(OR^{B})₂, or vice versa. Said group —B(OR^{B})₂ may be a boronic acid moiety (R^{B}=—H) or an alkyl ester of the boronic acid, e.g. its isopropyl ester (R^{B}=C₁-C₄-alkyl, e.g. —CH(CH₃)₂), or an ester derived from a diol such as e.g. pinacol in which the boronic acid intermediate forms a cyclic boronic ester, preferably a 4,4,5,5-tetramethyl-1,3,2-dioxaborolane (R^{B}-R^{B}=C₂-C₆-alkylene, preferably —C(CH₃)₂—C(CH₃)₂—). Many boronic acids and their esters are commercially available and their synthesis is well-known to the person skilled in the art; see e.g. D. G. Hall, Boronic Acids, 2005 WILEY-VCH Verlag GmbH & Co. KGaA, Weinheim, ISBN 3-527-30991-8 and references cited therein, and Journal of Medicinal Chemistry, 2015, 58, 2180-2194. Alternatively to boronic acid derivatives, also tetrafluoroborates, in which —BF₄⁻ replaces the —B(OR^{B})₂ moiety, can also be employed.

Said Suzuki coupling reaction can be catalysed by palladium catalysts, exemplified by but not limited to by Pd(0) catalysts such as e.g. tetrakis(triphenylphosphine)palladium(0) [Pd(PPh₃)₄], tris(dibenzylideneacetone)di-palladium(0) [Pd₂(dba)₃] in combination with a ligand, e.g. a phosphine such as e.g. triphenylphosphine, or by Pd(II) catalysts such as e.g. dichlorobis(triphenylphosphine)-palladium(II) [Pd(PPh₃)₂Cl₂], dichloropalladium-tricyclohexylphosphine (1:2), palladium(II) acetate in combination with a ligand, e.g. a phosphine such as e.g. triphenylphosphine, chloro(2-dicyclohexylphosphino-2′,4′,6′-triisopropyl-1,1′-biphenyl)[2-(2′-amino-1,1′-biphenyl)]palladium(II) (herein also referred to as XPhos Pd G2), (2-Dicyclohexylphosphino-2′,4′,6′-triisopropyl-1,1′-biphenyl)[2-(2′-amino-1,1′-biphenyl)]palladium(II) methanesulfonate (herein also referred to as XPhos Pd G3), or by [1,1′-bis(diphenylphosphino)ferrocene]-palladium dichloride, in free form [Pd(dppf)Cl₂] or as complex with dichloromethane [Pd(dppf)Cl₂×CH₂Cl₂].

The reaction is preferably carried out in solvents such as e.g. 1,2-dimethoxyethane, 1,4-dioxane, DMF, THF, or n-propanol, or mixtures thereof, optionally also in mixture with water, and in the presence of a base such as e.g. aqueous potassium carbonate, aqueous sodium carbonate or aqueous potassium triphosphate.

The reaction is performed at temperatures ranging from room temperature (i.e. 20° C.) to the boiling point of the solvent. Additionally, the reaction can be performed at temperatures above the boiling point using pressure tubes and a microwave oven. (for a review on Suzuki couplings see: D. G. Hall, Boronic Acids, 2005 WILEY-VCH Verlag GmbH & Co. KGaA, Weinheim, ISBN 3-527-30991-8 and references cited therein).

The reaction is preferably completed after 1 to 36 hours of reaction time.

Synthetic approaches to starting materials of formulae (VI) and (VII) are discussed in paragraph D. of this chapter, infra.

Compounds of formula (II) can be obtained using various methods described in more detail below, e.g. by reacting compounds of formula (V) with compounds of formula (IV) in which LG represents a leaving group, preferably bromo or iodo, and in which R^{p2} represents a group suitable to act as a precursor for the group R⁶ as defined for the compounds of general formula (I). The following paragraphs outline more specific examples of said conversion of compounds of formulae (Va) and (Ve), both constituting sub-compartments of formula (V), into compounds of (IIa), (IIc), and (IIe), all of them constituting sub-compartments of formula (II), some of which with the intermediacy of compounds of formulae (IIIb) and (IIIc), both of them constituting sub-compartments of formula (III), as discussed in the context of Scheme 1.

Said macrocyclic intermediates of formula (II) can finally be converted into the compounds of general formula (I) as described in further detail in context with Scheme 3, infra.

B. More Specific Synthesis Routes for Establishing the Macrocyclic Core, Schemes 2a-2e:

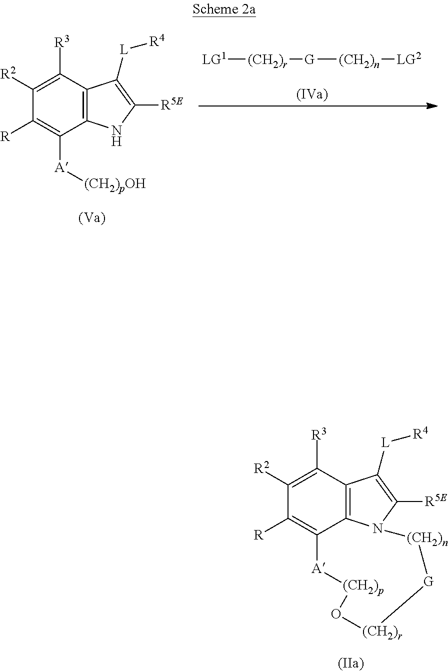

According to Scheme 2a, compounds of formula (IIa), in which R⁷ (which is a feature of group A as defined for the compounds of general formula (I)) and R⁶ together form a ^{##}—(CH₂)ₚ—O—(CH₂)ᵣ-G-(CH₂)ₙ—^{#} group, in which ^{#} represents the point of attachment to the indole nitrogen atom and ^{##} represents the point of attachment to the pyrazole carbon atom bearing the R⁷ substituent, can be obtained from compounds of formula (Va), in which R¹, R², R³, R⁴ and L are as defined for the compounds of general formula (I), in which R^{5E} represents a group suitable to act as a precursor of a —C(═O)OH or a tetrazol-5-yl group, preferably a group —C(═O)O—C₁₋₄-alkyl, in which R^{p2} represents a hydrogen atom and R^{p1} (see General Synthesis Route, Scheme 1) represents a —(CH₂)ₚ—OH group, in which index “p” is as defined for the compounds of general formula (I), by reacting with compounds of formula (IVa), in which G and indices “r” and “n”, are as defined for the compounds of general formula (I), with the proviso that the sum of the integers represented by indices “p”, “r” and “n” is at least 2 and does not exceed 9, and LG¹ and LG², independently from each other, represent a leaving group, preferably chloro, bromo or iodo, to give rise to the corresponding macrocyclic intermediates of formula (IIa).

The abovementioned transformation can be advantageously accomplished by deprotonating a compound of formula (Va) with a suitable base, such as e.g. cesium carbonate, in a suitable solvent, such as e.g. tetrahydrofuran, acetonitrile, or a mixture thereof, followed by addition of a compound of formula (IVa), preferably at a temperature between 50° C. and 80° C. In order to enhance reactivity of said compound of formula (IVa), LG¹ and/or LG², if e.g. representing chloro, can be interconverted into iodo in situ by adding an alkali iodide, such as e.g. sodium iodide, to the reaction mixture. Specific examples are given in the Experimental section, infra. The reader is referred to the fact that, whenever indices “r” and “n” are different from each other, and also dependent of the symmetry properties of the group G, mixtures of regioisomers can be formed, which can be separated by methods known to the person skilled in the art, such as preparative HPLC.

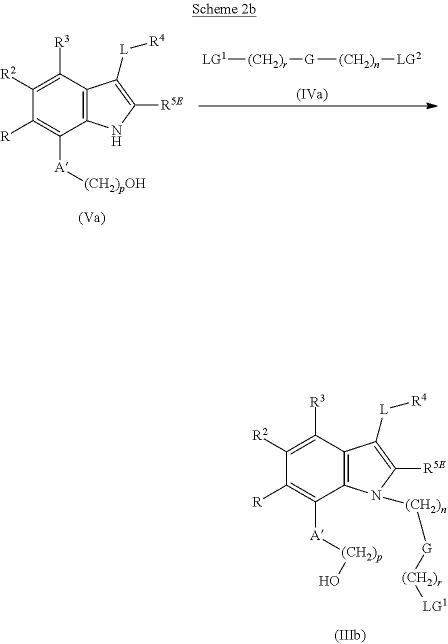

The reader is further referred to the fact, that—dependent on the reaction conditions and the reactivities of the reactive groups involved, that is, in particular, the hydroxy group attached to—(CH₂)ₚ— and LG¹, the reaction between compounds of formula (Va) with compounds of formula (IVa), as shown and discussed in context of Scheme 2a, may not proceed to macrocyclisation but results in the formation of non-macrocyclic intermediates of formula (IIIb), as shown above in Scheme 2b.

Said transformation can be advantageously accomplished by deprotonating a compound of formula (Va) with a suitable base, such as e.g. cesium carbonate, in a suitable solvent, such as e.g. THF, acetonitrile, or a mixture thereof, followed by addition of a compound of formula (IVa), in which both LG¹ and LG² preferably represent bromo, and by performing said reaction preferably at a temperature between 20° C. and 40° C., particularly at ambient temperature. Specific examples are given in the Experimental section, infra.

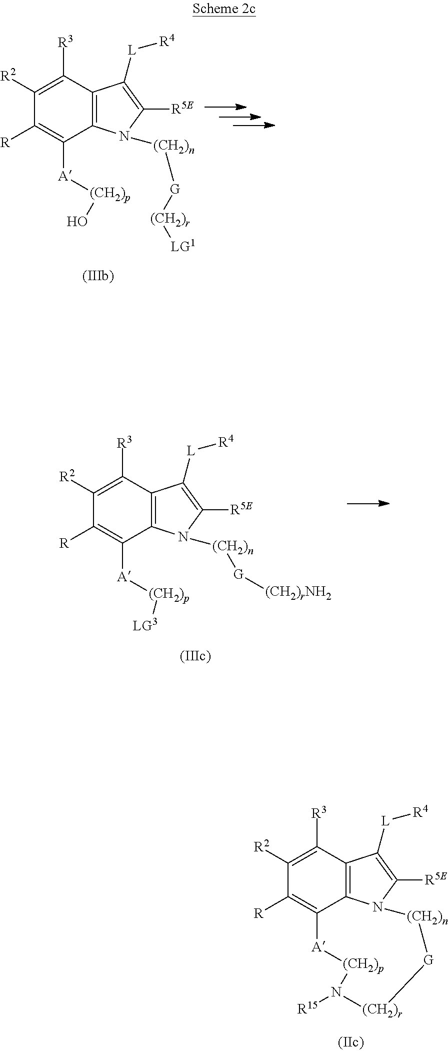

According to Scheme 2c, compounds of formula (IIc), in which R⁷ (which is a feature of group A as defined for the compounds of general formula (I)) and R⁶ together form a ^{##}—(CH₂)ₚ—N(R¹⁵)—(CH₂)ᵣ-G-(CH₂)ₙ—^{#} group, in which ^{#} represents the point of attachment to the indole nitrogen atom and ^{##} represents the point of attachment to the pyrazole carbon atom bearing the R⁷ substituent, can be obtained from compounds of formula (IIIb) described in the context of the preceding Schemes 2a and 2b, in which R¹, R², R³, R⁴, G, L, and the indices “n”, “r” and “p” are as defined for the compounds of general formula (I), with the proviso that the sum of the integers represented by indices “p”, “r” and “n” is at least 2 and does not exceed 9, and in in which R^{5E} represents a group suitable to act as a precursor of a —C(═O)OH or a tetrazol-5-yl group, preferably a group —C(═O)O—C₁₋₄-alkyl, and in which LG¹ represents a leaving group, by (i) reacting with compounds of formula HN(PG¹)₂, in which PG¹ represents a protective group for amino groups as defined herein, preferably a tert-butoxycarbonyl group, in the presence of a base such as sodium hydride, in a suitable solvent such as N,N-dimethylformamide (DMF), (ii) conversion of the —(CH₂)ₚ—OH group present in formula (IIIb) into a —(CH₂)ₚ-LG³ group, by methods well known to the person skilled in the art, and (iii), cleavage of said protective groups PG¹, to give compounds of formula (IIIc), in which R^{p1} represents a —(CH₂)ₚ-LG³ group (in which in turn LG³ represents a leaving group, preferably bromo), and in which R^{p2} represents a —(CH₂)ₙ-G-(CH₂)ᵣ—NH₂ group. Dependent on the reaction and/or work-up conditions, compounds of the formula (IIIc) can be isolated as free bases or as salts, e.g. salts with hydrochloric acid. Subsequently, said compounds of formula (IIIa) can be subjected to an intramolecular nucleophilic substitution, in the presence of a suitable base, giving rise to the corresponding macrocyclic intermediates of formula (IIc), in which R¹⁵ represents hydrogen. R¹⁵ groups different from a hydrogen atom can be introduced by various methods well known to the person in the art, to be selected according to the desired structure of said group R¹⁵.

The abovementioned sequence of transformations can be advantageously accomplished by (step i) deprotonating a compound of formula (IIIb) with a suitable base, such as e.g. sodium hydride, in a suitable solvent, such as e.g. DMF, followed by addition of a compound of formula HN(PG¹)₂, performing said reaction preferably at a temperature between 10° C. and 50° C., subsequently (step ii) by halogenation of said —(CH₂)ₚ—OH group, e.g. by treatment with tetrabromomethane and triphenylphosphine in a halogenated hydrocarbon, such as e.g. dichloromethane, as a solvent, and (step iii), cleavage of said protective groups PG¹ by an appropriate deprotection method (see e.g. T. W. Greene and P. G. M. Wuts in Protective Groups in Organic Synthesis, 4^{th} edition, Wiley 2006), such as the cleavage of a tert-butoxycarbonyl group by hydrogen chloride in dioxane or by trifluoroacetic acid. The subsequent macrocyclization is favourably accomplished by reacting a compound of formula (IIIc) in the presence of a base such as an alkali carbonate or an alkali phosphate, preferably cesium carbonate, in a solvent such as e.g. acetonitrile, N,N-dimethylformamide (DMF), N,N-dimethylacetamide or N-methyl pyrrolidin-2-one, preferably acetonitrile, preferably at a temperature between 30° C. and 70° C. Advantageous methods for the introduction of R¹⁵ groups different from a hydrogen atom in the compounds of formula (IIc) include but are not limited to reactions of a compound of formula (IIc), in which R¹⁵ represents a hydrogen atom, with
an aldehyde selected from HCHO and C₁-C₅-alkyl-CHO in the presence of sodium cyanoborohydride (to introduce R¹⁵=C₁-C₆-alkyl),
a compound C₁-C₃-alkyl-COOH in the presence of a suitable amide coupling agent and a base such as a tertiary aliphatic amine of the formula (C₁-C₃-alkyl)₃N,
an isocyanate, such as a compound heterocyclyl-N═C═O or heterocyclyl-(C₁-C₃-alkylene)-N═C═O, in the presence of a base such as a tertiary aliphatic amine of the formula (C₁-C₃-alkyl)₃N,
a sulfonyl halide, such as a compound heterocycloalkyl-(C₁-C₃-alkylene)-SO₂—Cl, in the presence of a base such as a tertiary aliphatic amine of the formula (C₁-C₃-alkyl)₃N.

Specific examples are given in the Experimental section, infra.

The reader is referred to the fact that this synthesis route, giving rise to compounds in which B represents a —NR¹⁵— group and t represents the integer 1, can be modified in various ways to enable the introduction of other groups B, inter alia by oxidising said —(CH₂)ₚ—OH to a carboxy or carboxyalkyl group (which mandates “p” to be different from 0), followed e.g. amide coupling with an amino group as present in formula (IIIc), or by replacing HN(PG¹)₂ through different reagents, e g. suitable for the introduction of —S—, —S(O)— and —S(O)₂, such as sodium sulfide, e.g. by reacting a group —SH, formed through said reaction with sodium sulfide, with a suitable reaction partner such as LG¹ or LG³, as described supra, analogously to what is discussed in context of Scheme 2c, optionally followed by oxidation to the corresponding sulfoxide and/or sulfone, by methods well known to the person skilled in the art, after accomplished macrocyclisation.

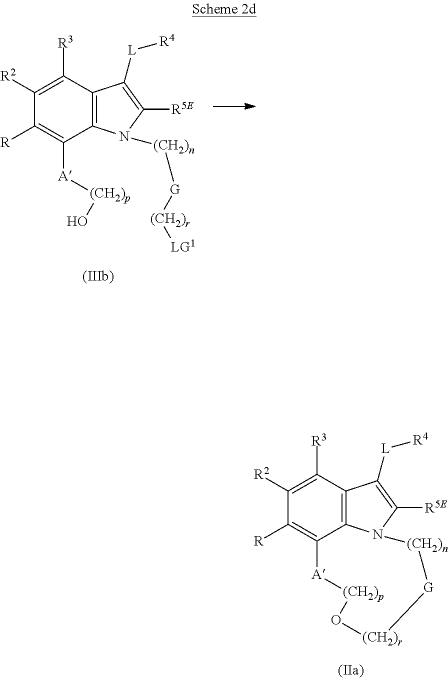

The reader is further referred to the fact that the intermediates of formula (IIIb) as described and discussed in the context of the preceding Schemes 2b and 2c, may also undergo intramolecular nucleophilic substitution e.g. when being reacted with compounds of formula HN(PG¹)₂, in which PG¹ represents a protective groups for amino groups as defined herein, preferably a tert-butoxycarbonyl group, in the presence of a base such as e.g. sodium hydride, in a solvent such as e.g. tetrahydrofurane or N,N-dimethylformamide (DMF), in competition or complete replacement of the reaction with the anion of HN(PG¹)₂, as discussed in context of Scheme 2c, giving rise to macrocyclic intermediates of the formula (IIa). Specific examples are given in the Experimental section, infra.

It is readily recognised by the person skilled in the art that said intramolecular nucleophilic substitution only mandates the presence of a base and a solvent, but not the presence of further reactants such as said compounds of formula HN(PG¹)₂.

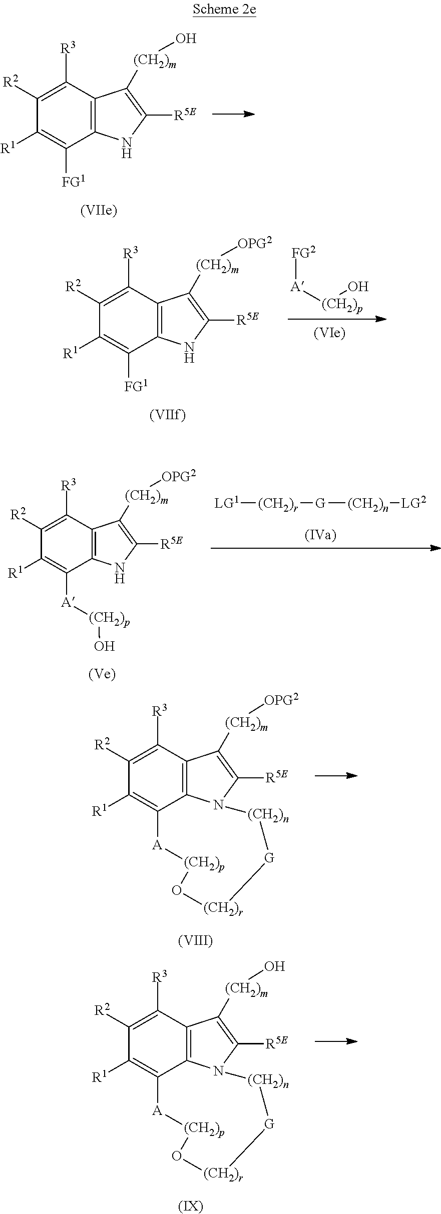

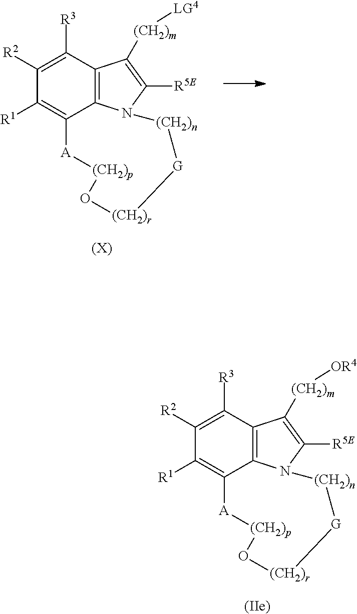

Scheme 2e outlines a modified general synthesis route for certain macrocyclic intermediates of general formula (IIe), constituting a sub-compartment of formula (II), supra, in which E represents an oxygen atom, which employs indole starting materials of formula (VIIe). The approach differs from the ones described in the preceding Schemes 2a-2d in that the group R⁴ is only introduced on late stage, after elaboration of the macrocyclic core, and hence is particularly useful for preparing multiple compounds of the present invention with many different R⁴ groups.

As shown in Scheme 2e, indole starting materials of formula (VIIe), in which R¹, R², R³, and m are as defined for the compounds of general formula (I), in which R^{5E} represents a group suitable to act as a precursor of a —C(═O)OH or a tetrazol-5-yl group, preferably a group —C(═O)O—C₁₋₄-alkyl, and in which FG¹ represents chloro, bromo, iodo, a trifluoromethanesulfonyl- group, or a group —B(OR^{B})₂, preferably bromo or iodo, more preferably a group —B(OR^{B})₂, are protected at their free hydroxy group attached to —(CH₂)ₘ— with PG², a protective group for hydroxy groups as defined herein, such as e.g. tert-butyldimethylsilyl-, by reaction with a suitable reagent such as e.g. tert-butylchlorodimethylsilane, in the presence of a base such as e.g. imidazole, using a halogenated aliphatic hydrocarbon, such as e.g. dichloromethane, as a solvent, to give indole derivatives of formula (VIIf). It is well possible to elaborate said —B(OR^{B})₂ group, if not present already in the compounds of formula (VIIe), from bromo upon introduction of the protective group PG². Specific examples are given in the Experimental Section, infra. In formulae (VIe), (VIIe) and (VIIf), FG¹ in combination with FG² represents a pair of functional groups together enabling a Suzuki coupling; either FG¹ represents chloro, bromo, iodo or a trifluoromethanesulfonyl- group, preferably bromo or iodo, and FG² represents a group —B(OR^{B})₂, or vice versa. Said group —B(OR^{B})₂ may be a boronic acid moiety (R^{B}=—H) or an alkyl ester of the boronic acid, e.g. its isopropyl ester (R^{B}=C₁-C₄-alkyl, e.g. —CH(CH₃)₂), or an ester derived from a diol such as e.g. pinacol in which the boronic acid intermediate forms a cyclic boronic ester, preferably a 4,4,5,5-tetramethyl-1,3,2-dioxaborolane (R^{B}-R^{B}=C₂-C₆-alkylene, preferably —C(CH₃)₂—C(CH₃)₂—).

Said indole derivatives of formula (VIIf) can, in analogy to the methods discussed in the context of Scheme 1, be reacted in a well-known Suzuki coupling with compounds of formula (VIe), in which p is as defined for the compound of general formula (I), in which FG² is as discussed above and in and in which A′, together with the group —(CH₂)ₚ—OH attached to it, represents a group suitable to act as precursor of a group A as defined for the compounds of general formula (I), to give compounds of formula (Ve). Said indole starting materials of formula (VIIe) are well known to the person skilled in the art and can be prepared as described infra.

In a subsequent step, the macrocyclic core can be elaborated using approaches such as those outlined and discussed in the context of Scheme 2a, by reacting said compounds of formula (Ve) with compounds of formula (IVa), in which G and indices “r” and “n”, are as defined for the compounds of general formula (I), with the proviso that the sum of the integers representing indices “p”, “r” and “n” is at least 2 and does not exceed 9, and LG¹ and LG², independently from each other, represent a leaving group, preferably chloro, bromo or iodo, to furnish macrocyclic intermediate compounds of formula (VIII). It is readily recognised by the person skilled in the art that the elaboration of the macrocycle can also follow different routes, e.g. in analogy to Scheme 2c, thus allowing to introduce groups —B—, as defined for the compounds of general formula (I), which are different from —O—.

Said macrocyclic intermediate compounds of formula (VIII) can be subsequently subjected to a cleavage of the protective group PG², according to methods known to the person skilled in the art (see e.g. T. W. Greene and P. G. M. Wuts in Protective Groups in Organic Synthesis, 4^{th} edition, Wiley 2006), e.g. by reacting with tetrabutylammonium fluoride in tetrahydrofuran in case PG² represents a tert-butyldimethylsilyl- group, to give compounds of the formula (IX). The hydroxy group present in said compounds of the formula (IX) can then be converted into LG⁴, representing a leaving group as defined herein, by methods known to the person skilled in the art, such as the reaction with tetrabromomethane in the presence of triphenylphosphane, in a suitable solvent such as a halogenated aliphatic hydrocarbon, e.g. dichloromethane, giving rise to compounds of the formula (X). The group R⁴ can finally be introduced by reaction of said compounds of the formula (X) with a compound of the formula R⁴—OH, in which R⁴ is as defined for the compounds of formula (I), in the presence of a base, such as e.g. sodium hydride or cesium carbonate, in a solvent such as e.g. tetrahydrofurane or N,N-dimethylformamide (DMF), to give compounds of formula (IIe). Specific examples are given in the Experimental section, infra.

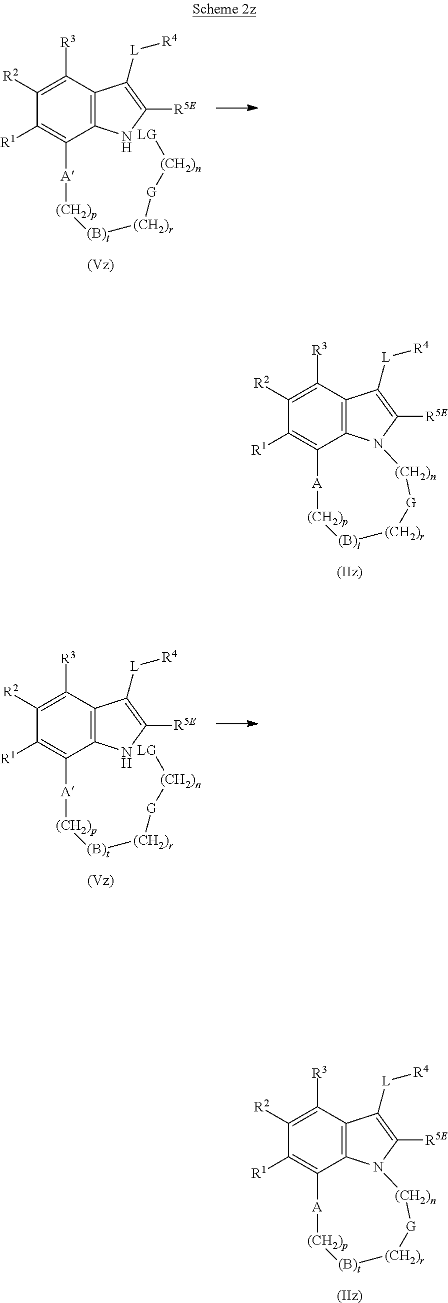

According to Scheme 2z, compounds of formula (IIz), in which R⁷ (which is a feature of group A as defined for the compounds of general formula (I)) and R⁶ together form a ^{#}—(CH₂)ₙ-(G)-(CH₂)ᵣ—(B)ₜ—(CH₂)ₚ—^{##} group, in which A, B, G, t, n, p and r are as defined for the compounds of the general formula (I), ^{#} represents the point of attachment to the indole nitrogen atom and ^{##} represents the point of attachment to the pyrazole carbon atom bearing the R⁷ substituent, can be obtained from compounds of formula (Vz), in which A′ is A or represents a group suitable to act as precursor of a group A as defined for the compounds of general formula (I), in a sense that it is only differing from A as its substituent R⁷ is not yet forming a ring together with R⁶ of the indol nitrogen atom substituent, in which R¹, R², R³, R⁴, B, G, t, n, p and r are as defined for the compounds of general formula (I), in which R^{5E} represents a group suitable to act as a precursor of a —C(═O)OH or a tetrazol-5-yl group, preferably a group —C(═O)O—C₁₋₄-alkyl, in which R^{p2} represents a hydrogen atom and R^{p1} (see General Synthesis Route, Scheme 1) represents a —(CH₂)ₚ—(B)ₜ—(CH₂)ᵣ-G-(CH₂)ₙ-LG group, in which LG represents a suitable leaving group, preferably in which LG represents —OH, —Br, —OSO₂CF₃ or —OSO₂CH₃, more preferably in which LG represents —OH, by direct alkylation of the indole nitrogen atom, such as by nucleophilic substitution or preferably by reacting in a so-called Mitsunobu reaction (see e.g. O. Mitsunobu, Synthesis 1981, 1, 1-28) with an azodicarboxylate of the formula C₁-C₄-alkyl-O₂C—N═N—CO₂—C₁-C₄-alkyl, preferably diisopropyl azodicarboxylate or di-tert-butyl azodicarboxylate, and a phosphine (R^{P})₃P, in which the three groups R^{P} are independently selected from each other from C₁-C₄-alkyl, phenyl, and furan-2-yl, whereby phenyl is optionally substituted one or two times with C₁-C₃-alkyl, C₁-C₃-alkoxy, or halogen; giving rise to the corresponding macrocyclic intermediates of formula (IIz). Optionally, cyanomethylene phosphoranes of the formula NC—C═PR^{P}₃ can be used, wherein the three groups R^{P} are as defined above, preferably wherein the three groups R^{P} are selected from C₁-C₄-alkyl, more preferably wherein the three groups R^{P} are n-butyl. Cyanomethylene phosphoranes are easily accessible to a skilled person via literature procedures (see e.g. T. Tsunoda, Tetrahedron Lett. 1994, 35, 5081) and/or commercially available.

Said reaction can be advantageously accomplished in a solvent selected from an acyclic or cyclic ether, such as e.g. tetrahydrofurane, tetrahydropyrane, 1,2-dimethoxyethane, bis-(2-methoxymethyl) ether, diethyl ether, or in a dipolar aprotic solvent, such as e.g. N,N-dimethylformamide, N,N-dimethylacetamide or acetonitrile, or an aliphatic halogenated hydrocarbon of the formula C₁-C₃-haloalkyl-H, such as e.g. dichloromethane, chloroform, or 1,2-dichloroethane, at a temperature in a range from 0° C. to 60° C. Preferably, the reaction is carried out in tetrahydrofurane at room temperature, that is, in a temperature range from 20° C. to 25° C. Phosphines (R^{P})₃P and azodicarboxylates of the formula C₁-C₄-alkyl-O₂C—N═N—CO₂—C₁-C₄-alkyl are widely commercially available.

Specific examples are given in the Experimental section, infra.

C. Conversion into Compounds of Formula (I), Scheme 3:

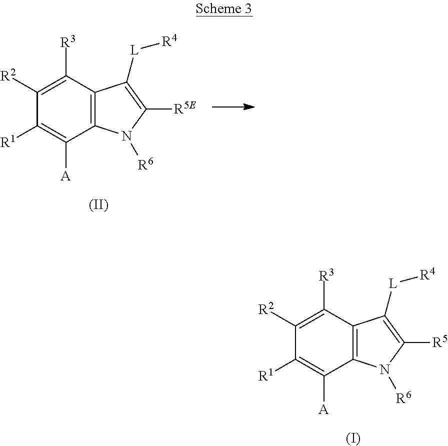

According to Scheme 3, compounds of formula (II) (such as the compounds of the formulae (IIa), (IIc) and (IIe)), in which R¹, R², R³, R⁴, R⁶, A and L are as defined for the compounds of general formula (I), and in which R^{5E} represents a group suitable to act as a precursor of a —C(═O)OH or a tetrazol-5-yl group, preferably a carboxylic ester group, such as e.g. a —C(═O)O—C₁₋₄-alkyl group or a benzyl ester, can be readily converted into compounds of formula (I) by transforming group R^{5E} into group R⁵ as defined for the compounds of general formula (I), preferably by reacting with an alkali hydroxide, such as e.g. potassium hydroxide, sodium hydroxide, lithium hydroxide, preferably lithium hydroxide, in a mixture of water with THF and/or an aliphatic alcohol of the formula C₁-C₃-alkyl-OH, preferably methanol or ethanol, at a temperature between 0° C. and 100° C., and subsequent usual workup as known by the person skilled in the art and as for example disclosed in the experimental section.

Said compounds of general formula (I) may be obtained as free acids or converted into pharmaceutically acceptable salts thereof, such as alkali salts, e.g. sodium or potassium salts, earth alkali salts, e.g. magnesium or calcium salts, and ammonium salts, e.g. ammonium (NH₄⁺), diethylammonium (herein also referred to as N-ethylethanamine salts) or triethylammonium salts, by methods known to the person skilled in the art. Compounds of the invention featuring a basic nitrogen atom, such as some of those obtainable from macrocyclic intermediates of formula (IIc), can be isolated as salts with a counteranion of the basic nitrogen, such as trifluoroacetate, and the like, or as inner carboxylate salts. Further, compounds of formula (I) in which R⁵ represents a free carboxylic acid group can be optionally converted into an acylsulfonamide according to methods known to the person skilled in the art (see for example: Bioorg. Med Chem. Lett. 2006, 16, 3639-3641; Bioorg. Med Chem. Lett. 2012, 22, 713-717; Org. Lett. 2012, 14(2), 556-559).

Further, single enantiomers of said compounds of general formula (I) may be obtained by methods known to the person skilled in the art, such as preparative HPLC on a chiral stationary phase, as described supra, and as exemplified in the Experimental Section, infra.

D. Synthesis Routes to Starting Materials of Formulae (VI) and (VII); Schemes 4a-4b:

As outlined in Schemes 4a and 4b below, several approaches, which are intended to illustrate but not to limit the synthetic routes available to the person skilled in the art for this purpose, can be followed in order to prepare starting materials of the formula (VI), as defined in the context of Scheme 1, supra, i.e. in which A′, together with the group R^{p1} attached to it, represents a group suitable to act as precursor of a group A as defined for the compounds of general formula (I), and in which FG², in combination with the group FG¹ present in formula (VII), represents a pair of functional groups together enabling a Suzuki coupling; either FG¹ represents chloro, bromo, iodo or a trifluoromethanesulfonyl- group, preferably bromo or iodo, and FG² represents a group —B(OR^{B})₂ as defined supra, or vice versa. Preferably, FG² represents bromo. Conversion of compounds, in which FG² represents bromo, into compounds in which FG² represents a group —B(OR^{B})₂, is possible on various steps of the outlined synthesis routes using methods well known to the person skilled in the art.

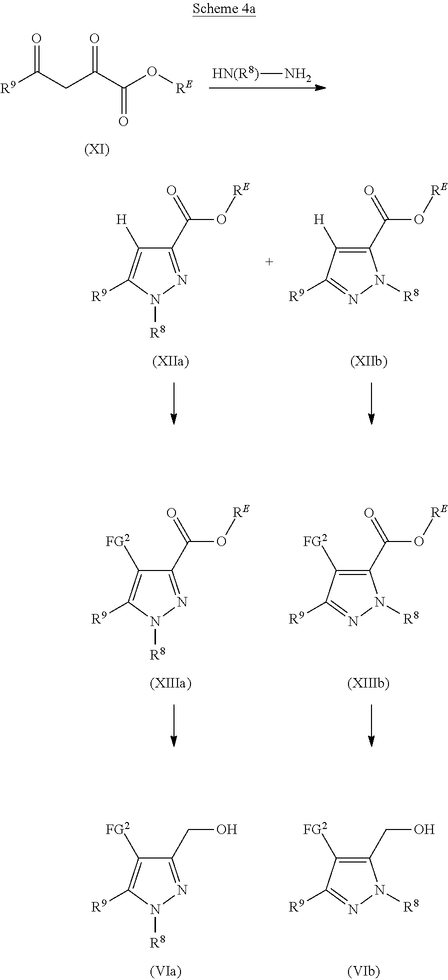

Scheme 4a illustrates the synthesis route enabling the preparation of compounds of formula (VI), in which A′ is derived from pyrazole, namely compounds of formulae (VIa) and (VIb), both of them constituting sub-compartments for formula (VI).

Said compounds of formulae (VIa) and (VIb) can be prepared from well-known α,γ-diketoesters of formula (XI), in which R⁹ is as defined for the compounds of general formula (I), and in which R^{E} represents a C₁-C₆-alkyl group, by reaction with hydrazines HN(R⁸)—NH₂, in which R⁸ is as defined for the compounds of general formula (I), to give regioisomeric mixtures of pyrazole derivatives of formulae (XIIa) and (XIIb), which can be separated on this step or on one of the steps described below. Said hydrazines of the formula HN(R⁸)—NH₂ are well known to the person skilled in the art, and are widely commercially available. If unsubstituted hydrazine (R⁸=H) is used, R⁸ groups different from a hydrogen atom can be introduced into compounds of formulae (XIIa) and (XIIb) e.g. by suitable alkylating agents such as a C₁-C₆-alkyl halide or a di(C₁-C₆-alkyl)sulfate in the presence of a base, such as sodium carbonate, in a solvent such as dichloromethane or N,N-dimethylformamide.

Said pyrazole derivatives of formulae (XIIa) and (XIIb) can subsequently be reacted with reagents suitable to introduce FG², such as N-halo succinimides or solutions of elemental halogens, to give pyrazole derivatives of formulae (XIIIa) and (XIIIb); preferably, N-bromo succinimide in a halogenated hydrocarbon, such as e.g. 1,2-dichloroethane, as a solvent, or bromine in a solvent such as e.g. glacial acetic acid or a halogenated hydrocarbon, such as dichloromethane, can be used. Said pyrazole derivatives of formulae (XIIIa) and (XIIIb) can subsequently be reduced by a suitable reducing agent not interfering with the groups FG², such as e.g. lithium borohydride, in a solvent such as e.g. tetrahydrofurane, to give pyrazolyl methanols of formulae (VIa) and (VIb). Specific examples are given in the Experimental section, infra. It is readily recognised by the person skilled in the art that the —CH₂OH group present in said pyrazolyl methanols of formulae (VIa) and (VIb) can be converted in various other R^{p1} groups (see formula (VI)).

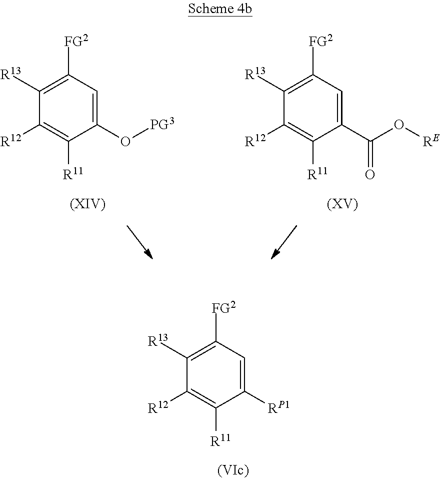

Scheme 4b illustrates synthesis routes enabling the preparation of compounds of formula (VI), in which A′ is derived from phenyl, pyridinyl, pyrimidinyl or pyridazinyl, namely compounds of formula (VIc), constituting yet another sub-compartment of formula (VI).

Starting from compounds of formula (XIV), in which R¹¹, R¹², and R¹³ are as defined for the compounds of general formula (I), and wherein one or two of the groups selected from CR¹¹, CR¹² or CR¹³ may be replaced by a nitrogen atom, and in which FG², in combination with the group FG¹ present in formula (VII), represents a pair of functional groups together enabling a Suzuki coupling; either FG¹ represents chloro, bromo, iodo or a trifluoromethanesulfonyl- group, preferably bromo or iodo, and FG² represents a group —B(OR^{B})₂ as defined supra, or vice versa, and in which PG³ represents a protective group, compounds of formula (VIc), in which R^{p1} represents a hydroxy group, can be readily obtained. Likewise, compounds of formula (XV), in which R¹¹, R¹², and R¹³ are as defined for the compounds of general formula (I), and wherein one or two of the groups selected from CR¹¹, CR¹² or CR¹³ may be replaced by a nitrogen atom, and in which FG², in combination with the group FG¹ present in formula (VII), represents a pair of functional groups together enabling a Suzuki coupling; either FG¹ represents chloro, bromo, iodo or a trifluoromethanesulfonyl- group, preferably bromo or iodo, and FG² represents a group —B(OR^{B})₂ as defined supra, or vice versa, and R^{E} represents a group —C₁-C₆-alkyl, can be converted into compounds of formula (VIc), in which R^{p1} represents a —CH₂—OH group, a —C(═O)H group, or a —CH₂-LG⁵ group, in which LG⁵ represents a leaving group, preferably bromo, in analogy to methods known to the person skilled in the art.

Compounds of formulae (XIV) and (XV) are commercially available, and known to the person skilled in the art, in considerable variety. Using known methods, groups R¹¹, R¹² and R¹³ can be broadly modified using known methods at various stages of the synthesis. Protective groups as present in compounds of formula (XIV), and methods of their removal, are well known to the person skilled in the art, see e.g. T. W. Greene and P. G. M. Wuts in Protective Groups in Organic Synthesis, 4^{th} edition, Wiley 2006.

Indole based starting materials of formula (VII), in which R¹, R², R³, R⁴ and L are as defined for the compounds of general formula (I), in which R^{5E} represents a group suitable to act as a precursor of a —C(═O)OH or a tetrazol-5-yl group, preferably a group —C(═O)O—C₁₋₄-alkyl, and in which FG¹ represents chloro, bromo, iodo, a trifluoromethanesulfonyl- group, or a group —B(OR^{B})₂, preferably bromo or iodo, more preferably a group —B(OR^{B})₂, can be prepared using methods well known to the person skilled in the art, see e.g. Journal of Medicinal Chemistry, 2015, 58, 2180-2194. Said group —B(OR^{B})₂ may be a boronic acid moiety (R^{B}=—H) or an alkyl ester of the boronic acid, e.g. its isopropyl ester (R^{B}=C₁-C₄-alkyl, e.g. —CH(CH₃)₂), or an ester derived from a diol such as pinacol in which the boronic acid intermediate forms a cyclic boronic ester, preferably a 4,4,5,5-tetramethyl-1,3,2-dioxaborolane (R^{B}-R^{B}=C₂-C₆-alkylene, preferably —C(CH₃)₂—C(CH₃)₂—). Alternatively to boronic acid derivatives, also tetrafluoroborates, in which —BF₄⁻ replaces the —B(OR^{B})₂ moiety, can also be employed.

Modification of any of the substituents, such as R¹, R², R³, R⁴, R⁵, R^{5E}, R⁶, R⁷, R⁸, R⁹, R¹⁰, R¹¹, R¹², R¹³, R^{p1} and R^{p2} can be achieved before and/or after the exemplified transformation. However, also other routes may be used to synthesise the target compounds, in accordance with common general knowledge of a person skilled in the art of organic synthesis. Also, suitable and optionally protected precursor groups of said substituents can be carried through the synthesis routes described in context of the Schemes above, to be elaborated into the actual substituents as defined for the general formula (I) on late stage, as exemplified e.g. for R⁴ in Intermediates 59 to 103 in the Experimental Section below.

Said modifications can be such as the introduction of protective groups, cleavage of protective groups, reduction or oxidation of functional groups, formation or cleavage of esters or carboxamides, halogenation, metallation, substitution or other reactions known to a person skilled in the art. These transformations include those which introduce a functionality which allows for further interconversion of substituents. Appropriate protective groups and their introduction and cleavage are well-known to a person skilled in the art (see for example T. W. Greene and P. G. M. Wuts in Protective Groups in Organic Synthesis, 4^{th} edition, Wiley 2006). Further, it is possible that two or more successive steps may be performed without work-up being performed between said steps, e.g. a “one-pot” reaction, as it is well-known to a person skilled in the art.

In accordance with a further aspect, the present invention provides a method of preparing a compound of general formula (I) according to any one of claims 1 to 6, said method comprising the step of reacting an intermediate compound of general formula (II)

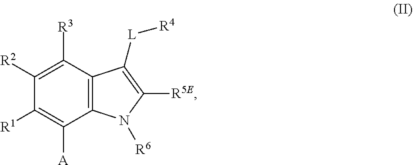

wherein R¹, R², R³, R⁴, R⁶, A and L are as defined for the compound of general formula (I) according to any one of claims 1 to 5, and R^{5E} represents a carboxylic ester group, such as e.g. a —C(═O)O—C₁₋₄-alkyl group or a benzyl ester with an alkali hydroxide in a mixture of water and tetrahydrofuran and/or an aliphatic alcohol of formula C₁-C₃-alkyl-OH, at a temperature from 0° C. to 100° C., to transform the group R^{5E} into a group R⁵ as defined for the compounds of general formula (I), isolating and optionally purifying the compound of formula (I) to obtain a compound of general formula (I)

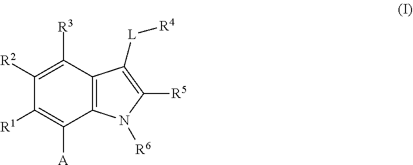

wherein R¹, R², R³, R⁴, R⁵, R⁶, A and L are as defined for the compound of general formula (I) according to any one of claims 1 to 6 or a stereoisomer, a tautomer, an N-oxide, a hydrate, a solvate, or a salt of a stereoisomer, a salt of a tautomer, or a salt of an N-oxide thereof, or a mixture of same and/or optionally converting the free acid group R⁵ into a pharmaceutically acceptable salt thereof and/or subsequently, or optionally preceding the salt formation step, optionally separating enantiomers by means of preparative HPLC on a chiral stationary phase.

In accordance with a further aspect, the present invention covers a method of preparing compounds of general formula (I) according to any one of claims 1 to 6, said method comprising the step of reacting an intermediate compound of general formula (II)

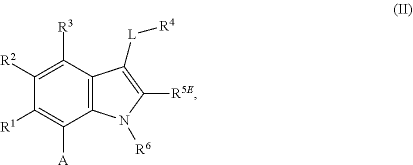

wherein R¹, R², R³, R⁴, R⁶, A and L are as defined for the compound of general formula (I) according to any one of claims 1 to 5, and R^{5E} represents a —C(═O)O—C₁₋₄-alkyl group or a benzyl ester with an alkali hydroxide such as e.g. potassium hydroxide, sodium hydroxide, lithium hydroxide, preferably lithium hydroxide, in a mixture of water and THF and/or an aliphatic alcohol of the formula C₁-C₃-alkyl-OH, preferably methanol or ethanol, at a temperature from 0° C. to 100° C., preferably from 20° C. to 60° C., to transform the group R^{5E} into a group R⁵ as defined for the compounds of general formula (I), and subsequently optionally to convert the free acid group R⁵ into a pharmaceutically acceptable salts thereof to obtain a compound of general formula (I)

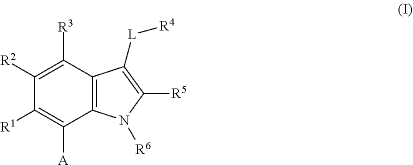

wherein R¹, R², R³, R⁴, R⁵, R⁶, A and L are as defined for the compound of general formula (I) according to any one of claims 1 to 5 or a stereoisomer, a tautomer, an N-oxide, a hydrate, a solvate, or a salt thereof, or a mixture of same and subsequently optionally separating enantiomers by means of preparative HPLC on a chiral stationary phase.

In accordance with a further aspect, the present invention provides a method of preparing a compound of general formula (I) according to any one of claims 1 to 6, said method comprising the step of reacting an intermediate compound of general formula (Va)

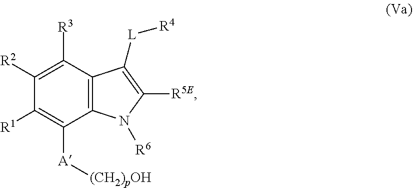

wherein R¹, R², R³, R⁴ and L as well as p are as defined for the compounds of general formula (I), in which R^{5E} represents a group suitable to act as a precursor of a —C(═O)OH or a tetrazol-5-yl group, preferably a group —C(═O)O—C₁₋₄-alkyl, and A′—(CH₂)ₚ—OH represents a group suitable to act as precursor of a group A as defined for the compounds of general formula (I) in a sense that it is only differing from A as its substituent R⁷ is not yet forming a ring together with R⁶ of the indol nitrogen atom substituent,

with a compound of formula (IVa)

LG¹-(CH₂)ᵣG-(CH₂)ₙ-LG² (IVa)

in which G and indices “r” and “n”, are as defined for the compounds of general formula (I), with the proviso that the sum of the integers represented by indices “p”, “r” and “n” is at least 2 and does not exceed 9, and LG¹ and LG², independently from each other, represent a leaving group, under basic conditions in a polar solvent or mixture of solvents at temperatures of 50° C.-80° C. (the limits being included), optionally adding an alkali iodide, subsequently isolating the compound of formula (Va), optionally separating isomers obtained and optionally subsequently saponifying the ester group R^{5E} with the method mentioned above in order to obtain a compound of general formula (I).

In accordance with a further aspect, the present invention provides the intermediate compound of formula (Va).

In accordance with a further aspect, the present invention provides the intermediate compound of formula (IVa).

The present invention provides methods of preparing compounds of the present invention of general formula (I), said methods comprising the steps as described in the Experimental Section herein.

In accordance with a further aspect, the present invention provides intermediate compounds which are useful for the preparation of the compounds of general formula (I), supra.

Particularly, the invention provides the intermediate compounds of general formula (II)

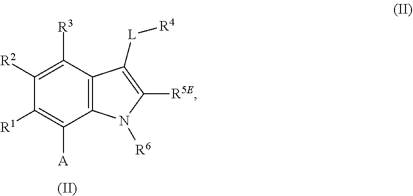

in which R¹, R², R³, R⁴, R⁶, A and L are as defined for the compound of general formula (I) according to any one of claims 1 to 5, and R^{5E} represents a carboxylic ester such as e.g. a —C(═O)O—C₁₋₄-alkyl group or a benzyl group.

In accordance with another aspect, the present invention provides the use of said intermediate compounds for the preparation of a compound of general formula (I) as defined supra.

In accordance with another aspect, the present invention provides a method of using the intermediate compound of general formula (II) for the preparation of a compound of general formula (I).

The present invention provides the intermediate compounds which are disclosed in the Example Section of this text, infra.

The present invention provides any sub-combination within any embodiment or aspect of the present invention of intermediate compounds of general formula (II), supra.

The compounds of general formula (I) of the present invention can be converted to any salt, preferably pharmaceutically acceptable salts, as described herein, by any method which is known to the person skilled in the art. Similarly, any salt of a compound of general formula (I) of the present invention can be converted into the free compound, by any method which is known to the person skilled in the art.

Methods and Administration

Compounds of general formula (I) of the present invention demonstrate a valuable pharmacological spectrum of action and pharmacokinetic profile, both of which could not have been predicted. Compounds of the present invention have surprisingly been found to effectively inhibit MCL-1 activity, and it is possible therefore that said compounds can be used for the treatment or prophylaxis of diseases, preferably hyperproliferative disorders in humans and animals.

As used herein, “prophylaxis” includes a use of the compound that, in a statistical sample, reduces the occurrence of the disorder or condition in the treated sample relative to an untreated control sample, or delays the onset or reduces the severity of one or more symptoms of the disorder or condition relative to the untreated control sample, when administered to prior to the onset of the disorder or condition.

Compounds of the present invention can be utilized to inhibit, block, reduce, and/or decrease cell proliferation and/or cell division, and/or induce apoptosis. Disclosed methods include administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalamic glioma, cerebellar and cerebral astrocytoma, medulloblastoma, ependymoma, as well as neuroectodermal and pineal tumours.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, basal cell carcinoma, squamous cell carcinoma, Kaposi's sarcoma, malignant melanoma, Merkel cell skin cancer, and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Sarcomas include, but are not limited to, sarcoma of the soft tissue, osteosarcoma, malignant fibrous histiocytoma, lymphosarcoma, and rhabdomyosarcoma.

Leukemias include, but are not limited to acute lymphoblastic leukemia, acute myeloid leukemia, (acute) T-cell leukemia, acute lymphoblastic leukemia, acute lymphocytic leukemia (ALL), acute monocytic leukemia (AML), acute promyelocytic leukemia (APL), bisphenotypic B myelomonocytic leukemia, chronic lymphocytic leukemia, chronic myelogenous leukemia, chronic myeloid leukemia (CML), chronic myelomonocytic leukemia (CMML), large granular lymphocytic leukemia, plasma cell leukemia, and also myelodysplastic syndrome (MDS), which can develop into an acute myeloid leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al, New Engl. J. Med., 1994, 331, 1480; Peer et al., Lab. Invest., 1995, 72, 638], age-related macular degeneration (AMD) [Lopez et al., Invest. Opththalmol. Vis. Sci., 1996, 37, 855], neovascular glaucoma, psoriasis, retrolental fibroplasias, angiofibroma, inflammation, rheumatoid arthritis (RA), restenosis, in-stent restenosis, and vascular graft restenosis. In addition, the increased blood supply associated with cancerous and neoplastic tissue encourages growth, leading to rapid tumour enlargement and metastasis. Moreover, the growth of new blood and lymph vessels in a tumour provides an escape route for rapidly dividing cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, and/or decreasing endothelial cell proliferation, or other pathways involved in angiogenesis, as well as causing cell death or apoptosis of such cell types.

These disorders have been well characterized in humans, but also exist with a similar etiology in other mammals, and can be treated by administering pharmaceutical compositions of the present invention.

By “subject” is meant a mammal, including, but not limited to, a human or non-human mammal, such as a bovine, equine, canine, ovine, or feline.

The term “treating” or “treatment” as stated throughout this document is used conventionally, for example the management or care of a subject for the purpose of combating, alleviating, reducing, relieving, and/or improving the condition of a disease or disorder, such as a carcinoma.

The compounds of the present invention can be used in particular in therapy and prevention, i.e., prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemotherapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In some embodiments of the present invention, the compounds of general formula (I) of the present invention may be used to sensitize a cell to radiation, i.e., treatment of a cell with a compound of the present invention prior to radiation treatment of the cell renders the cell more susceptible to DNA damage and cell death than the cell would be in the absence of any treatment with a compound of the present invention. In some embodiments, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In some embodiments, after the cell is treated with one or more compounds of general formula (I) of the present invention, the cell is treated with at least one compound, or at least one method, or a combination thereof, in order to cause DNA damage for the purpose of inhibiting the function of the normal cell or killing the cell.

In other embodiments of the present invention, a cell is killed by treating the cell with at least one DNA damaging agent, i.e., after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g., cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In some embodiments, a compound of general formula (I) of the present invention is administered to a cell prior to the radiation or other induction of DNA damage in the cell. In some embodiments, a compound of general formula (I) of the present invention is administered to a cell concomitantly with the radiation or other induction of DNA damage in the cell. In some embodiments, a compound of general formula (I) of the present invention is administered to a cell immediately after radiation or other induction of DNA damage in the cell has begun.

In some embodiments, the cell is in vitro. In some embodiments, the cell is in vivo.

Thus in some embodiments, the present invention includes a method of inhibiting proliferation of a cell and/or the induction of apoptosis in a cell, comprising contacting the cell with a compound of formula (I) according to any one of claims 1-6.

Furthermore in some embodiments, the present invention includes a method of using a compound of general formula (I) for the treatment of diseases.

Particularly in some embodiments, the present invention includes a method of treating a hyperproliferative disease, more particularly cancer, comprising administering an effective amount of at least one compound of general formula (I) according to any one of claims 1-6.

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, ovarian cancer, comprising administering an effective amount of at least one compound of formula (I) according to any one of claims 1-6.

Furthermore in some embodiments, the present invention includes a method of treating cancer, particularly multiple myeloma, ovarian carcinoma, acute monocytic leukemia, melanoma and lung cancer. comprising administering an effective amount of at least one compound of formula (I) according to any one of claims 1-6.

In some embodiments the present invention provides for compounds of general formula (I) for use in a method of treating cancer, particularly where the cancer disease is multiple myeloma, ovarian carcinoma, acute monocytic leukemia, melanoma and lung cancer.

Furthermore in some embodiments, the present invention includes a method of treating cancer, particularly breast cancer; lung cancer; lymphoma including non-Hodgkin-lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; ovarian cancer; pancreas cancer comprising administering an effective amount of at least one compound of formula (I) according to any one of claims 1-6. GC-DLBCL means Germinal B-cell Diffuse Large B-Cell Lymphoma and ** ABC-DLBCL means Activated B-cell Diffuse Large B-Cell Lymphoma.

Furthermore in some embodiments, the present invention includes a method of treating cancer, particularly breast cancer, lung cancer, diffuse large B-cell lymphoma subtype including GC-DLBCL* and ABC-DLBCL** subtypes, mantle cell lymphoma, acute monocytic leukemia, melanoma, ovarian cancer, pancreas cancer comprising administering an effective amount of at least one compound of formula (I) according to any one of claims 1-6. Furthermore in accordance with another aspect, the present invention provides a compound of formula (I) for use of treating diseases.

Furthermore in some embodiments, the present invention includes a method of treating cancer, particularly breast cancer; lymphoma, leukemia, multiple myeloma; and ovarian cancer comprising administering an effective amount of at least one compound of formula (I) according to any one of claims 1-6.

Furthermore in accordance with another aspect, the present invention provides a compound of formula (I) for use of treating diseases.

In some embodiments, the present invention includes a compound of general formula (I) for use in a method of inhibiting proliferation of a cell and/or the induction of apoptosis in a cell, comprising contacting the cell with a compound of formula (I) according to any one of claims 1-6.

Particularly in some embodiments, the present invention includes compounds of general formula (I) for use in a method of treating a hyperproliferative disease, more particularly wherein the hyperproliferative disease is cancer, and yet even more particularly wherein the cancer disease is lymphoma, non-Hodgkin-lymphoma type, diffuse large B-cell lymphoma subtype, ovarian cancer, multiple myeloma, acute leukemia, and acute myeloid leukemia.

More particularly in some embodiments, the present invention includes compounds of general formula (I) for use in a method of treating a hyperproliferative disease, more particularly wherein the hyperproliferative disease is cancer, and yet even more particularly wherein the cancer disease is breast cancer; lymphoma, leukemia, multiple myeloma; and ovarian cancer.

Particularly in some embodiments, the present invention includes compounds of general formula (I) for use in a method of treating a hyperproliferative disease, more particularly wherein the hyperproliferative disease is cancer, and yet even more particularly wherein the cancer disease breast cancer; esophageal cancer; liver cancer; lung cancer; lymphoma including non-Hodgkin-lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; melanoma; ovarian cancer; pancreas cancer.

More particularly in some embodiments, the present invention includes compounds of general formula (I) for use in a method of treating cancer whereby the cancer disease is selected from breast cancer; lymphoma, leukemia, multiple myeloma; and ovarian cancer.

In some embodiments, the present invention includes use of the compounds of general formula (I) for the manufacture of a medicament for the treatment of a hyperproliferative disease, particularly cancer and more particularly breast cancer; lymphoma, leukemia, multiple myeloma; and ovarian cancer.

In some embodiments, the present invention includes use of the compounds of general formula (I) for the manufacture of a medicament for the treatment of a hyperproliferative disease, particularly cancer and more particularly lymphoma, non-Hodgkin-lymphoma type, diffuse large B-cell lymphoma subtype, ovarian cancer, multiple myeloma, acute leukemia, and acute myeloid leukemia type.

It is possible for the compounds according to the invention to have systemic and/or local activity.

For this purpose, they can be administered in a suitable manner, such as, for example, via the oral, parenteral, pulmonary, nasal, sublingual, lingual, buccal, rectal, vaginal, dermal, transdermal, conjunctival, or otic route or as an implant or stent.

For these administration routes, it is possible for the compounds according to the invention to be administered in suitable administration forms.

For oral administration, it is possible to formulate the compounds according to the invention into dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatin capsules), sugar-coated tablets, granules, pellets, powders, emulsions, suspensions, aerosols or solutions. It is possible to incorporate the compounds according to the invention in crystalline and/or amorphous and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation (inter alia powder inhalers, nebulizers), nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example, cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example, polyethylene glycols, cacao butter, hard fat),
solvents (for example, water, ethanol, isopropanol, glycerol, propylene glycol, medium chain-length triglycerides, fatty oils, liquid polyethylene glycols, paraffins),
surfactants, emulsifiers, dispersants or wetters (for example, sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example, phosphates, carbonates, citric acid, acetic acid, hydrochloric acid, sodium hydroxide solution, ammonium carbonate, trometamol, triethanolamine),
isotonicity agents (for example, glucose, sodium chloride),
adsorbents (for example, highly-disperse silicas),
viscosity-increasing agents, gel formers, thickeners and/or binders (for example, polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatin),
disintegrants (for example, modified starch, carboxymethylcellulose-sodium, sodium starch glycolate (such as, for example, Explotab®), cross- linked polyvinylpyrrolidone, croscarmellose-sodium (such as, for example, AcDiSol®)),
flow regulators, lubricants, glidants and mould release agents (for example, magnesium stearate, stearic acid, talc, highly-disperse silicas (such as, for example, Aerosil®)),
coating materials (for example, sugar, shellac) and film formers for films or diffusion membranes which dissolve rapidly or in a modified manner (for example, polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example, gelatin, hydroxypropylmethylcellulose),
synthetic polymers (for example, polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example, polyethylene glycols, propylene glycol, glycerol, triacetine, triacetyl citrate, dibutyl phthalate),
penetration enhancers,
stabilisers (for example, antioxidants such as, for example, ascorbic acid, ascorbyl palmitate, sodium ascorbate, butylhydroxyanisole, butylhydroxytoluene, propyl gallate),
preservatives (for example, parabens, sorbic acid, thiomersal, benzalkonium chloride, chlorhexidine acetate, sodium benzoate),
colourants (for example, inorganic pigments such as, for example, iron oxides, titanium dioxide), and
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In some embodiments, the present invention includes pharmaceutical combinations, in particular medicaments, comprising at least one compound of general formula (I) of the present invention and at least one or more further active ingredients, in particular for the treatment and/or prophylaxis of a hyperproliferative disorder, particularly cancer.

Particularly, the present invention includes a pharmaceutical combination, which comprises:
one or more first active ingredients, in particular compounds of general formula (I) as defined supra, and
one or more further active ingredients, in particular for the treatment and/or prophylaxis of hyperproliferative disorder, particularly cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent, or in combination with one or more other pharmaceutically active ingredients where the combination causes no unacceptable adverse effects. The present invention also includes such pharmaceutical combinations. For example, the compounds of the present invention can be combined with known anti-cancer agents.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Based upon standard laboratory techniques known to evaluate compounds useful for the treatment of hyperproliferative diseases, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 40 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 3000 mg of active ingredient, and can be administered one or more times per day or less than once a day. The average daily dosage for administration by injection, including intravenous, intramuscular, subcutaneous and parenteral injections, and use of infusion techniques will preferably be from about 0.01 to about 200 mg/kg of total body weight. The average daily rectal dosage regimen will preferably be from about 0.01 to about 200 mg/kg of total body weight. The average daily vaginal dosage regimen will preferably be from about 0.01 to about 200 mg/kg of total body weight. The average daily topical dosage regimen will preferably be from about 0.1 to about 200 mg administered between one to four times daily. The transdermal concentration will preferably be that required to maintain a daily dose of from about 0.01 to about 200 mg/kg. The average daily inhalation dosage regimen will preferably be from about 0.01 to about 100 mg/kg of total body weight.

In one embodiment the average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from abut 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Experimental Section

### Experimental Section—NMR Spectra

To the extent NMR peak forms and multiplicities are specified, they are stated as they appear in the spectra, possible higher order effects have not been considered.

The ¹H-NMR data of selected examples are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight”<1%.

### Experimental Section—Abbreviations

The following table lists the abbreviations used in this paragraph and in the Intermediates and Examples section as far as they are not explained within the text body. Other abbreviations have their meanings customary per se to the skilled person. A comprehensive list of the abbreviations utilized by organic chemists of ordinary skill in the art appears presented in the first issue of each volume of the Journal of Organic Chemistry; this list is typically presented in a table titled “Standard List of Abbreviations”. In case of doubt, the abbreviations and/or their meaning according to the following table shall prevail.

TABLE 1
Abbreviations

| Abbreviation | Meaning |
|---|---|
| br. | broad signal (NMR) |
| BPR | Back Pressure Regulator |
| d | doublet (NMR) |
| DAD | Diode array detector |
| dd | doublet of doublet (NMR) |
| dt | doublet of triplet (NMR) |
| DMF | N,N-dimethylformamide |
| DMSO | dimethylsulfoxide |
| EDC | N-[3-(dimethylamino)propyl]-N′-ethyl- |
| | carbodiimide hydrochloride |
| EDTA | ethylenediaminetetraacetic acid |
| ee | enantiomeric excess |
| ESI | electrospray (ES) ionisation |
| h, hr (hrs) | hour(s) |
| HCl | hydrogen chloride, hydrochloric acid |
| HPLC | high performance liquid chromatography |
| HRP | horseradish peroxidase |
| LC-MS | liquid chromatography-mass spectrometry |
| m | multiplet (NMR) |
| Min | minute(s) |
| MS | mass spectrometry |
| MTP | microtiter plate |
| MWD | Multiple wavelength detector |
| Na—K-tartrate | Sodium potassium tartrate |
| NHS | N-hydroxysuccinimide |
| NMR | nuclear magnetic resonance spectroscopy: |
| | chemical shifts (δ) are given in ppm. |
| | The chemical shifts were corrected by |
| | setting the DMSO signal to 2.50 ppm using |
| | dmso-d6 unless otherwise stated. |
| NAD⁺ | nicotinamide adenine dinucleotide |
| PBS | phosphate buffered saline |
| Pd(dppf)Cl₂xCH₂Cl₂ | [1,1′-Bis-(diphenylphosphino)- |
| | ferrocen]-dichloropalladium(II), complex |
| | with dichloromethane |
| q | quartet (NMR) |
| quin | quintet (NMR) |
| rt | room temperature |
| Rt, Rt | retention time |
| s | singulet (NMR) |
| SFC | Supercritical Fluid Chromatography |
| SPA | Scintillation proximity assay |
| t | triplet (NMR) |
| td | triplet of doublet (NMR) |
| TFA | trifluoroacetic acid |
| THF | tetrahydrofuran |
| TLC | thin layer chromatography |
| UPLC | ultra performance liquid chromatography |
| UV | ultraviolet |
| wt-% | percent of weight |
| [³H]- | tritium |
| δ | chemical shift |
| XPhos Pd G2 | Chloro(2-dicyclohexylphosphino-2′-,4′-,6′- |
| | triisopropyl-1,1′-biphenyl)[2-(2′-amino- |
| | 1,1′-biphenyl)]palladium(II) (Cas No: |
| | 1310584-14-5) |
| XPhos Pd G3 | (2-Dicyclohexylphosphino-2′-,4′-,6′- |
| | triisopropyl-1,1′-biphenyl)[2-(2′-amino- |
| | 1,1′-biphenyl)]palladium(II) methane- |
| | sulfonate (Cas No: 1445085-55-1) |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art. Reactions were set up and started, e.g. by the addition of reagents, at temperatures as specified in the protocols; if no temperature is specified, the respective working step was performed at ambient temperature, i.e. between 18 and 25° C.

“Silicone filter” or “water resistant filter” refers to filter papers which are made hydrophobic (impermeable to water) by impregnation with a silicone. With the aid of these filters, water can be separated from water-immiscible organic solvents by means of a filtration (i.e. filter paper type MN 617 WA, Macherey-Nagel).

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be removed by trituration using a suitable solvent or solvent mixture. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4® or Isolera Four®) and eluents such as e.g. gradients of hexane/ethyl acetate or DCM/ethanol. In flash column chromatography, unmodified (“regular”) silica gel may be used as well as aminophase functionalized silica gel. As used herein, “Biotage SNAP cartridge silica” refers to the use of regular silica gel; “Biotage SNAP cartridge NH₂ silica” refers to the use of aminophase functionalized silica gel. If reference is made to flash column chromatography or to flash chromatography in the experimental section without specification of a stationary phase, regular silica gel was used. Further, column chromatography can also be used advantageously in the reversed-phase mode, using materials such as e.g. C18 silica gel as stationary phase, and using eluents such as e.g. gradients of water and acetonitrile which may contain additives such as e.g. trifluoroacetic acid, formic acid, diethylamine or aqueous ammonia. If reference is made to reversed phase column chromatography in the experimental section without specification of a stationary phase, C18 siliga gel was used.

In some cases, the compounds may be purified by preparative HPLC using for example a Waters autopurifier equipped with a diode array detector and/or on-line electrospray ionization mass spectrometer in combination with a suitable prepacked reverse phase column and eluents such as e.g. gradients of water and acetonitrile which may contain additives such as e.g. trifluoroacetic acid, formic acid, diethylamine or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

UPLC-MS Standard Procedures

Analytical UPLC-MS was performed as described below. The masses (m/z) are reported from the positive mode electrospray ionisation unless the negative mode is indicated (ESI−).

Analytical UPLC Methods:

Method 1:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

Method 2:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

Method 3:

Instrument: Waters Alliance HT; Column: Waters Cortecs 30 mm×3 mm×2.7 μm; eluent A: Water (MilliQ)+0.01 vol % formic acid, eluent B: acetonitrile+0.01 vol % formic acid; gradient: 0-1.7 min 3-95% B, 1.7-2.2 min 95% B; 2.3-2.5 3% B; flow: 1.75 mL/mn; temperature: 45° C.; DAD scan: 200-500 nm.

Method 4:

Instrument: Waters Alliance HT; Column: Waters Cortecs 30 mm×3 mm×2.7 μm; eluent A: Water (MilliQ)+0.01 vol % formic acid, eluent B: acetonitrile+0.01 vol % formic acid; gradient: 0-6.8 min 5-95% B, 6.8-7.3 min 95% B; 7.3-7.5 5% B; flow: 1.75 mL/mn; temperature: 45° C.; DAD scan: 200-500 nm.

Method 5: UPLC1-MS methods

Column: CSH C18 1.7 μm 2.1×50 mm; Waters Acquity UPLC system; Waters Acquity Binary pump (Flow 0.8 mL/min); Waters Acquity Autosampler; Waters Acquity QDA; Waters Acquity PDA (Total plot 210-350 nm)
UPLC1-MS (Long Acid)
Run Time: 4.60 min; Solvents: A) 0.1% formic acid in water, B) Acetonitrile+0.1% formic acid; Gradient: 2-95% B with A in 4.00 min, hold at 95% B 5% A to 4.60 min @ 0.8 ml/min, 40° C.
UPLC1-MS (Long Acid 50 to 95)
Run Time: 4.60 min; Solvents: A) 0.1% formic acid in water, B) Acetonitrile+0.1% formic acid; Gradient: 50-95% B with A in 4.00 min, hold at 95% B 5% A to 4.60 min @ 0.8 ml/min, 40° C.

Preparative HPLC Methods:

Method P1:

Instrument: Waters Autopurification MS SingleQuad; Column: Waters XBridge C18 5μ 100×30 mm; eluent A: water+0.1 vol % formic acid (99%), eluent B: acetonitrile; gradient: 0-5.5 min 5-100% B; flow 70 mL/min; temperature: 25° C.; DAD scan: 210-400 nm.

Method P2:

Instrument: Pump: Labomatic HD-5000 or HD-3000, Head HDK 280, low pressure gradient module ND-B1000; Manual injection valve: Rheodyne 3725i038; Detector: Knauer Azura UVD 2.15; Collector: Labomatic Labocol Vario-4000; Column: Chromatorex RP C-18 10 μm, 125×30 mm; solvent A: water+0.1 vol-% formic acid, solvent B: acetonitrile; gradient: 0.00-0.50 min 40% B (150 mL/min), 0.50-6.00 min 40-80% B (150 mL/min), 6.00-6.10 min 80-100% B (150 mL/min), 6.10-8.00 min 100% B (150 mL/min), UV-Detection.

Method P3:

Instrument: Pump: Labomatic HD-5000 or HD-3000, Head HDK 280, low pressure gradient module ND-B1000; Manual injection valve: Rheodyne 3725i038; Detector: Knauer Azura UVD 2.15; Collector: Labomatic Labocol Vario-4000; Column: Chromatorex R^{P} C-18 10 μm, 125×30 mm; solvent A: water+0.1 vol-% formic acid, solvent B: acetonitrile; gradient: 0.00-0.50 min 65% B (150 mL/min), 0.50-6.00 min 65-100% B (150 mL/min), 6.00-8.00 min 100% B (150 mL/min), UV-Detection.

Method P4:

Instrument: Pump: Labomatic HD-5000 or HD-3000, Head HDK 280, low pressure gradient module ND-B1000; Manual injection valve: Rheodyne 3725i038; Detector: Knauer Azura UVD 2.15; Collector: Labomatic Labocol Vario-4000; Column: XBridge, RP C18 5 μm, 100×30 mm; Solvent A: water+0.2 Vol-% ammonia (32%), Solvent B: acetonitrile, gradient: 0.00-2.00 min 10% B (60 mL/min), 2.00-14.00 min 10-50% B (60 mL/min), 14.00-14.10 min 50-100% B (60 mL/min), 14.10-17.00 min 100% B (60 mL/min), UV-Detection.

Specific Optical Rotation Methods:

Method O1:

Instrument: JASCO P2000 Polarimeter; wavelength 589 nm; temperature: 20° C.; integration time 10 s; path length 100 mm.

### INTERMEDIATES

### Intermediate 1

### Ethyl 3-[3-(naphthalen-1-yloxy)propyl]-7-(4,4,5,5-tetramethyl-1,3,2-dioxaborolan-2-yl)-1H-indole-2-carboxylate

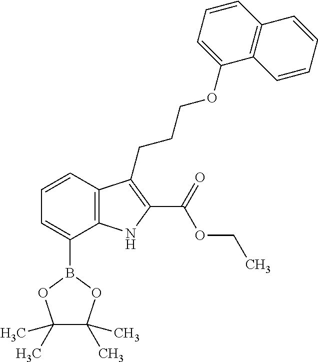 [SMILES: CCOC(=O)c1[nH]c2c(B3OC(C)(C)C(C)(C)O3)cccc2c1CCCOc1cccc2ccccc12]

Ethyl 3-[3-(naphthalen-1-yloxy)propyl]-7-(4,4,5,5-tetramethyl-1,3,2-dioxaborolan-2-yl)-1H-indole-2-carboxylate was prepared as described in the literature (Journal of Medicinal Chemistry, 2015, 58, 2180-2194).

### Intermediate 2

### Ethyl-7-bromo-6-chloro-3-(3-hydroxypropyl)-1H-indole-2-carboxylate

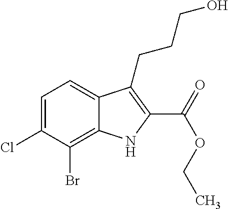 [SMILES: CCOC(=O)c1[nH]c2c(Br)c(Cl)ccc2c1CCCO]

The title compound was prepared as described in J. Med. Chem. 2015, 58, 3794-3805.

### Intermediate 3

### Ethyl 7-bromo-6-chloro-3-[3-(naphthalen-1-yloxy)propyl]-1H-indole-2-carboxylate

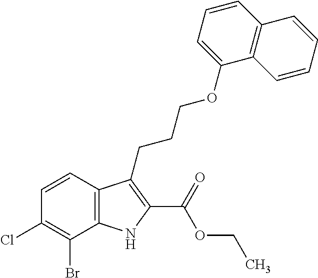 [SMILES: CCOC(=O)c1[nH]c2c(Br)c(Cl)ccc2c1CCCOc1cccc2ccccc12]

To a mixture of ethyl 7-bromo-6-chloro-3-(3-hydroxypropyl)-1H-indole-2-carboxylate (see intermediate 2, 6.62 g, 18.4 mmol), naphthalen-1-ol (CAS 90-15-3, 3.21 g, 99% purity, 22.0 mmol) and triphenylphosphine (5.84 g, 22.0 mmol) in THF (150 mL) was added diisopropyl azodicarboxylate (4.4 mL, 22 mmol) at 10° C. and the mixture was stirred for 24 hours at room temperature. For work-up, the mixture was diluted with ethyl acetate and was washed with aqueous sodium bicarbonate solution and brine. The organic phase was dried over sodium sulfate, filtered and the filtrate was concentrated under reduced pressure. The crude product was purified by flash chromatography (hexane/dichloromethane gradient 20→100% dichloromethane) to give, after subsequent trituration with methanol, the title compound (3.5 g).

### Intermediate 4

### Ethyl 7-bromo-6-chloro-3-{3-[(6-fluoronaphthalen-1-yl)oxy]propyl}-1H-indole-2-carboxylate

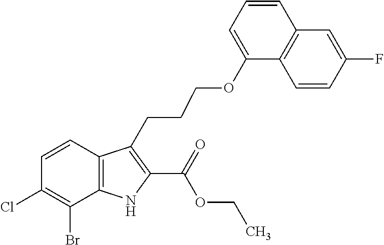 [SMILES: CCOC(=O)c1[nH]c2c(Br)c(Cl)ccc2c1CCCOc1cccc2cc(F)ccc12]

Triphenylphosphane (1.60 g, 6.10 mmol) was dissolved in 20 mL of THF and 6-fluoronaphthalen-1-ol (CAS 804498-72-4, 989 mg, 6.10 mmol) was added. This mixture was cooled to −10° C. and at this temperature diisopropyl azodicarboxylate (1.2 mL, 6.1 mmol) was added dropwise into the mixture. After complete addition it was stirred under cooling for additional 10 min. Ethyl 7-bromo-6-chloro-3-(3-hydroxypropyl)-1H-indole-2-carboxylate (see intermediate 2, 2.00 g, 5.55 mmol) was dissolved in 20 mL of THF and was added dropwise into the mixture under cooling. Afterwards the mixture was allowed to warm to rt and was stirred for overnight. The reaction mixture was concentrated under reduced pressure and the residue was triturated with methanol. The remaining solids were isolated by filtration.

In a second preparation, triphenylphosphane (1.60 g, 6.10 mmol) was dissolved in 20 mL of THF and 6-fluoronaphthalen-1-ol (CAS 804498-72-4, 989 mg, 6.10 mmol) was added. This mixture was cooled to −10° C. and at this temperature diisopropyl azodicarboxylate (1.2 mL, 6.1 mmol) was added dropwise into the mixture. After complete addition it was stirred under cooling for additional 10 min. Ethyl-7-bromo-6-chloro-3-(3-hydroxypropyl)-1H-indole-2-carboxylate (see intermediate 2, 2.00 g, 5.55 mmol) was dissolved in 20 mL of THF and was added dropwise into the mixture under cooling. Afterwards the mixture was allowed to warm to rt and was stirred for 3 days. The reaction mixture was concentrated under reduced pressure and the residue was triturated with methanol. The remaining solids were isolated by filtration.

In a third preparation, triphenylphosphane (1.60 g, 6.10 mmol) was dissolved in 20 mL of THF and 6-fluoronaphthalen-1-ol (CAS 804498-72-4, 989 mg, 6.10 mmol) was added. This mixture was cooled to −10° C. and at this temperature diisopropyl azodicarboxylate (1.2 mL, 6.1 mmol) was added dropwise into the mixture. After complete addition it was stirred under cooling for additional 10 min. Ethyl-7-bromo-6-chloro-3-(3-hydroxypropyl)-1H-indole-2-carboxylate (see intermediate 2, 2.00 g, 5.55 mmol) was dissolved in 20 mL of THF and was added dropwise into the mixture under cooling. Afterwards the mixture was allowed to warm to rt and was stirred overnight. The reaction mixture was concentrated under reduced pressure and the residue was triturated with methanol. The remaining solids were isolated by filtration. Combined with the products of the other preparations the crude material was purified by flash chromatography using silica gel (hexane/ethyl acetate gradient). The obtained material was triturated with a mixture of tert.-butyl methyl ether and petroleum ether and the remaining solids were filtered off and dried to give the title compound (2.4 g). The filtrate was concentrated and triturated with methanol. The remaining solids were filtered off and dried to give a second batch of the title compound (1.88 g).

LC-MS (Method 1): Rₜ=1.80 min, MS (ESIneg): m/z=502 [M−H]⁻

¹H-NMR (400 MHz, DMSO-d6) δ [ppm]: 1.191 (0.89), 1.208 (1.68), 1.226 (0.79), 1.275 (7.17), 1.286 (1.82), 1.293 (16.00), 1.303 (2.45), 1.310 (7.38), 1.321 (1.00), 2.177 (1.63), 2.194 (2.33), 2.211 (1.65), 2.227 (0.56), 2.518 (5.40), 2.523 (3.59), 3.280 (2.10), 3.299 (3.61), 3.317 (2.70), 4.147 (2.35), 4.162 (4.59), 4.176 (2.33), 4.190 (0.84), 4.269 (2.24), 4.286 (7.10), 4.304 (6.99), 4.322 (2.10), 5.759 (0.86), 6.834 (1.79), 6.842 (1.91), 6.848 (1.61), 6.856 (1.91), 7.168 (5.12), 7.189 (5.66), 7.240 (0.68), 7.249 (0.72), 7.261 (0.61), 7.271 (0.61), 7.316 (1.23), 7.322 (1.37), 7.333 (1.19), 7.339 (2.17), 7.345 (2.33), 7.360 (1.21), 7.367 (1.37), 7.391 (0.51), 7.412 (3.59), 7.418 (3.89), 7.426 (8.20), 7.438 (0.54), 7.579 (0.51), 7.600 (0.49), 7.624 (2.17), 7.630 (2.17), 7.650 (2.14), 7.656 (2.07), 7.721 (4.87), 7.743 (4.59), 7.757 (0.58), 7.778 (0.47), 8.046 (1.89), 8.061 (1.98), 8.069 (1.91), 8.084 (1.82), 11.517 (3.28).

On larger scale the title compound could be obtained in a similar manner with slightly modified reaction conditions in two batches: To a stirred solution of ethyl 7-bromo-6-chloro-3-(3-hydroxypropyl)-1H-indole-2-carboxylate (see Intermediate 6, 2000 g) and —N,N-diethylethanamine (1.77 kg) in dichloromethane (20.0 L) was added methanesulfonyl chloride (1.30 kg) dropwise over 3 hrs at 0-5° C. under an atmosphere of nitrogen. After addition, the reaction mixture was stirred at 25° C. for 16 hrs. The mixture was washed with water (8 L) and concentrated to give a brown solid (3.99 kg, crude). This material (697 g) was added to a stirred solution of 6-fluoronaphthalen-1-ol (CAS 804498-72-4, 214 g) and potassium carbonate (428 g) in acetonitrile (5400 mL) under an atmosphere of nitrogen, and the reaction mixture was stirred at 85° C. for 16 hrs. The mixture was filtered, and the solution was concentrated. The residue was purified by silica gel chromatography (petrol ether/dichloromethane=3/1) to obtain a crude material, which was then slurried in petrol ether/dichloromethane (800/200 mL) at 20° C. for 16 hrs, and was filtered to obtain the title compound (262 g).

### Intermediate 5

### Ethyl 6-chloro-3-[3-(naphthalen-1-yloxy)propyl]-7-(4,4,5,5-tetramethyl-1,3,2-dioxaborolan-2-yl)-1H-indole-2-carboxylate

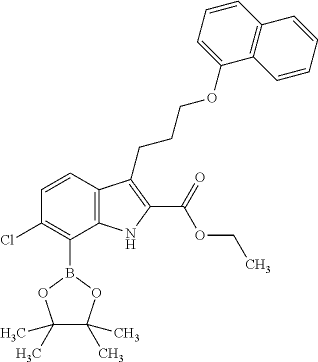 [SMILES: CCOC(=O)c1[nH]c2c(B3OC(C)(C)C(C)(C)O3)c(Cl)ccc2c1CCCOc1cccc2ccccc12]

To a degassed mixture of ethyl-7-bromo-6-chloro-3-[3-(naphthalen-1-yloxy)propyl]-1H-indole-2-carboxylate (see Intermediate 3, 5.50 g, 11.3 mmol) and 4,4,4′,4′,5,5,5′,5′-octamethyl-2,2′-bi-1,3,2-dioxaborolane (CAS 78183-34-3, 8.61 g, 33.9 mmol) in 1,4-dioxane (97 mL) was added potassium acetate (4.44 g, 45.2 mmol) and to the mixture was added 1,1′-Bis(diphenylphosphino)ferrocenpalladium(II)chloride (827 mg, 1.13 mmol), and the reaction mixture was purged with argon for 10 minutes. The mixture was stirred for 24 hours at 80° C. For work-up the mixture was concentrated and the residue was purified by flash chromatography (Biotage SNAP cartridge silica, hexane/ethyl acetate gradient, 7%->25% ethyl acetate) to give the title compound (1.5 g).

### Intermediate 6

### Ethyl 6-chloro-3-{3-[(6-fluoronaphthalen-1-yl)oxy]propyl}-7-(4,4,5,5-tetramethyl-1,3,2-dioxaborolan-2-yl)-1H-indole-2-carboxylate

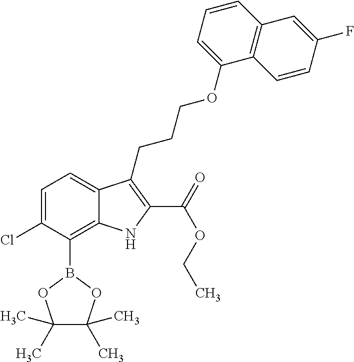 [SMILES: CCOC(=O)c1[nH]c2c(B3OC(C)(C)C(C)(C)O3)c(Cl)ccc2c1CCCOc1cccc2cc(F)ccc12]

Ethyl 7-bromo-6-chloro-3-{3-[(6-fluoronaphthalen-1-yl)oxy]propyl}-1H-indole-2-carboxylate (see intermediate 4, 200 mg, 396 μmol) was dissolved in 3 mL of DMF. 4,4,4′,4′,5,5,5′,5′-Octamethyl-2,2′-bi-1,3,2-dioxaborolane (CAS 78183-34-3151 mg, 594 μmol), potassium acetate (117 mg, 1.19 mmol) and [1,1′-Bis(diphenylphosphino)ferrocene]dichloro-palladium(II) (32.4 mg, 39.6 μmol) were added. The mixture was purged with argon for 10 min. The tube was sealed and the mixture was stirred at 95° C. for 12 hours. After cooling to rt the mixture was filtered and purified by preparative HPLC (method: P3) to give the title compound (34 mg, 12% yield).

LC-MS (Method 1): Rₜ=1.90 min, MS (ESIpos): m/z=552 [M+H]⁺

¹H-NMR (400 MHz, DMSO-d6) δ [ppm]: 1.257 (2.02), 1.275 (4.69), 1.292 (2.08), 1.324 (2.13), 1.373 (2.56), 1.383 (16.00), 2.202 (0.58), 2.219 (0.41), 2.518 (1.69), 2.522 (1.13), 3.301 (0.50), 3.320 (1.26), 4.144 (0.54), 4.159 (1.11), 4.173 (0.52), 4.235 (0.54), 4.252 (1.83), 4.270 (1.80), 4.288 (0.51), 5.758 (0.73), 6.824 (0.49), 6.832 (0.50), 6.838 (0.42), 6.846 (0.53), 7.045 (1.22), 7.067 (1.18), 7.319 (0.49), 7.326 (0.53), 7.408 (0.98), 7.414 (1.04), 7.422 (2.36), 7.622 (0.57), 7.629 (0.58), 7.648 (0.58), 7.655 (0.57), 7.839 (0.81), 7.861 (0.72), 8.010 (0.44), 8.024 (0.47), 8.033 (0.47), 8.047 (0.45), 9.978 (0.64).

On larger scale the title compound could be obtained in a similar manner with slightly modified reaction conditions: To a stirred solution of 4,4,4′,4′,5,5,5′,5′-octamethyl-2,2′-bi-1,3,2-dioxaborolane (CAS 78183-34-3151 mg, 91.7 g), sodium carbonate (76.6 g) and [1,1′-bis(diphenylphosphino)ferrocene]dichloro-palladium(II) (4.40 g) in 1,4-dioxane (700 mL) was added ethyl-7-bromo-6-chloro-3-{3-[(6-fluoronaphthalen-1-yl)oxy]propyl}-1H-indole-2-carboxylate (see Intermediate 8, 70 g) under an atmosphere of nitrogen, and the reaction mixture was stirred at 100° C. for 40 hrs. The residue was slurried in a mixture of ethanol and dichloromethane (300 and 50 mL) at 20° C. for 16 hrs, then recrystallized in dichloromethane (80 mL) from 50° C. to 0° C. for 3 hrs, and filtered to obtain the title compound (84.8 g). The filtrate was purified by silica gel column chromatography (petrol ether/ethyl acetate/dichloromethane=20/0/0-20/1/1) to obtain the title compound (21.2 g).

### Intermediate 7

### Ethyl 5-ethyl-1-[2-(morpholin-4-yl)ethyl]-1H-pyrazole-3-carboxylate

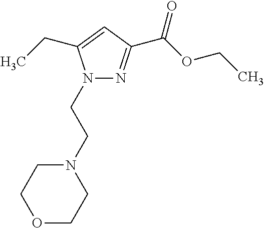 [SMILES: CCOC(=O)c1cc(CC)n(CCN2CCOCC2)n1]

To a mixture of ethyl 2,4-dioxohexanoate (CAS 13246-52-13.00 g, 17.1 mmol) in acetic acid (24 mL) was added 4-(2-hydrazinylethyl)morpholine (CAS 2154-24-7, 2.53 g, 17.1 mmol) at 0° C. and the reaction mixture was stirred at 100° C. for 3 h. Upon cooling, the mixture was concentrated. The residue was diluted with ethyl acetate and the organic phase was washed with saturated aqueous sodium bicarbonate solution and brine. The organic phase was dried over sodium sulfate. After filtration and removal of the solvents the crude product was purified by flash chromatography (hexane/ethyl acetate gradient, 50%->100% ethyl acetate) to give the title compound (1.96 g).

¹H-NMR (400 MHz, DMSO-d6) δ [ppm]: 1.192 (5.34), 1.211 (12.80), 1.230 (6.02), 1.249 (7.04), 1.267 (16.00), 1.285 (7.15), 2.385 (2.88), 2.396 (3.98), 2.408 (3.12), 2.518 (1.26), 2.523 (0.85), 2.642 (3.19), 2.658 (5.49), 2.661 (5.21), 2.663 (4.60), 2.669 (1.00), 2.675 (2.92), 2.680 (3.82), 2.682 (3.66), 2.700 (1.08), 3.519 (4.16), 3.530 (5.19), 3.542 (4.09), 4.175 (2.39), 4.192 (4.57), 4.207 (3.68), 4.225 (6.71), 4.242 (6.56), 4.260 (1.95), 6.511 (5.60).

### Intermediate 8

### Ethyl 3-ethyl-1-[2-(morpholin-4-yl)ethyl]-1H-pyrazole-5-carboxylate

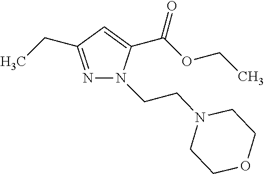 [SMILES: CCOC(=O)c1cc(CC)nn1CCN1CCOCC1]

The title compound was isolated as a side product in the synthesis of ethyl 5 ethyl-1-[2-(morpholin-4-yl)ethyl]-1H-pyrazole-3-carboxylate (see intermediate 7).

¹H-NMR (500 MHz, DMSO-d6) δ [ppm]: 0.000 (5.34), 1.145 (7.12), 1.155 (0.41), 1.160 (16.00), 1.175 (7.30), 1.283 (6.71), 1.298 (14.69), 1.312 (7.24), 1.906 (0.75), 2.367 (2.57), 2.376 (3.50), 2.384 (2.63), 2.529 (1.62), 2.544 (4.72), 2.559 (4.59), 2.574 (1.47), 2.602 (2.50), 2.615 (4.09), 2.629 (2.56), 3.329 (3.25), 3.501 (3.51), 3.510 (4.71), 3.519 (3.52), 4.255 (2.16), 4.269 (6.47), 4.284 (6.75), 4.298 (2.08), 4.507 (2.42), 4.520 (4.07), 4.534 (2.37), 6.653 (7.25).

### Intermediate 9

### Ethyl 4-bromo-3-ethyl-1-[2-(morpholin-4-yl)ethyl]-1H-pyrazole-5-carboxylate

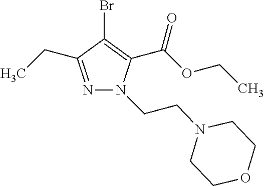 [SMILES: CCOC(=O)c1c(Br)c(CC)nn1CCN1CCOCC1]

A solution of bromine in acetic acid (8.2 mL, 1.0 M, 8.2 mmol) was added to a solution of ethyl 3-ethyl-1-[2-(morpholin-4-yl)ethyl]-1H-pyrazole-5-carboxylate (770 mg, 2.74 mmol; see intermediate 8) in acetic acid (16 mL) at 0° C., and the mixture was stirred for 4 h at room temperature. For work-up, the reaction was poured into ice water followed by the addition of a saturated aqueous sodium thiosulfate solution and the pH of the mixture was adjusted to pH >7 by the addition of saturated aqueous sodium bicarbonate solution. The mixture was extracted with ethyl acetate and the combined organic phases were dried over sodium sulfate. After filtration and removal of the solvents the crude product was purified by flash chromatography (hexane/ethyl acetate gradient, 0%->100% ethyl acetate) to give the title compound (810 mg).

LC-MS (Method 2): Rt=1.28 min; MS (ESIpos): m/z=360 [M+H]⁺

¹H-NMR (400 MHz, CDCl₃): δ [ppm]=4.61 (t, 2H), 4.39 (q, 2H), 3.75-3.53 (m, 4H), 2.71 (t, 2H), 2.64 (q, 2H), 2.51-2.36 (m, 4H), 1.59 (s, 2H), 1.43 (t, 3H), 1.23 (t, 3H)

### Intermediate 10

### {4-Bromo-3-ethyl-1-[2-(morpholin-4-yl)ethyl]-1H-pyrazol-5-yl}methanol

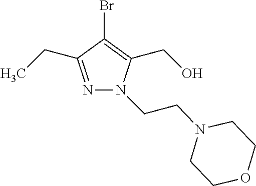 [SMILES: CCc1nn(CCN2CCOCC2)c(CO)c1Br]

To a solution of ethyl 4-bromo-3-ethyl-1-[2-(morpholin-4-yl)ethyl]-1H-pyrazole-5-carboxylate (810 mg, 2.25 mmol; see intermediate 9) in THF (9 mL) was added a solution of lithium borohydride in THF (1.3 mL, 2.0 M, 2.7 mmol) and the mixture was stirred at 60° C. for 24 hours. For work-up, sodium sulfate hydrate was added and the mixture was stirred for 1 hour at room temperature. The mixture was filtered and the filtrate was concentrated under reduced pressure and the crude product was purified by flash chromatography (dichloromethane/acetone gradient, 0%->40% acetone) to give the title compound (650 mg).

### Intermediate 11

### Ethyl 4-bromo-1,5-dimethyl-1H-pyrazole-3-carboxylate

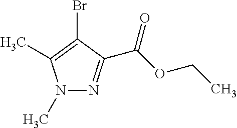

N-Bromosuccinimide (16.3 g, 90.5 mmol) was added to a solution of ethyl 1,5-dimethyl-1H-pyrazole-3-carboxylate (7.25 g, 43.1 mmol, CAS No 5744-51-4) in 1,2-dichloroethane (150 mL) and the mixture was stirred for 15 h at 80° C. For work-up, the mixture was diluted with dichloromethane, washed with water and the organic phase was filtered through a silicone filter and concentrated. The residue was purified by flash chromatography (Biotage SNAP cartridge silica 340 g, hexane/dichloromethane gradient, 0->100% dichloromethane) to give the title compound (6.49 g, 61% yield).

¹H-NMR (400 MHz, DMSO-d6) δ [ppm]: 1.261 (4.14), 1.278 (8.78), 1.296 (4.21), 2.268 (14.94), 2.518 (0.74), 2.523 (0.49), 3.857 (16.00), 4.229 (1.31), 4.247 (4.03), 4.264 (3.94), 4.282 (1.24).

### Intermediate 12

### (4-Bromo-1,5-dimethyl-1H-pyrazol-3-yl)methanol

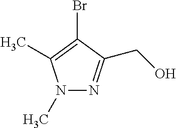

Lithium borohydride (711 mg, 32.6 mmol) was added to a solution of ethyl 4-bromo-1,5-dimethyl-1H-pyrazole-3-carboxylate (see intermediate 11, 6.45 g, 26.1 mmol) in THF (150 mL) and the mixture was stirred for 1 h at room temperature, followed by stirring for 7 h at 60° C. The reaction was stopped by addition of saturated aqueous ammonium chloride solution and the mixture was extracted with ethyl acetate. The organic phase was filtered through a silicone filter and concentrated. The residue was purified by flash chromatography (hexane/ethyl acetate gradient, 50%->100% ethyl acetate) to give the title compound (4.07 g, 76% yield).

¹H-NMR (400 MHz, DMSO-d6) δ [ppm]: 2.205 (16.00), 2.518 (0.43), 3.330 (10.35), 4.285 (3.97), 4.299 (4.13), 4.933 (1.00), 4.946 (2.22), 4.960 (0.93).

### Intermediate 13

### Ethyl 5-ethyl-1-methyl-1H-pyrazole-3-carboxylate

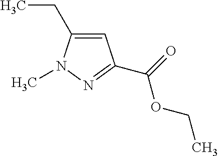

Ethyl-2,4-dioxohexanoate (CAS 13246-52-1, 5.00 g, 29.0 mmol) was dissolved in 20 mL of acetic acid. Under ice cooling, methylhydrazine (1.5 mL, 29.0 mmol) was added and the mixture was stirred at rt for 23 hours. Methylhydrazine (0.5 mL, 10.0 mmol) was added and stirring was continued at rt for 24 hours. The reaction mixture was poured into ice water and extracted with ethylacetate. The combined organic layers were washed with brine, dried using a water resistant filter, and concentrated under reduced pressure. The crude material was purified by flash chromatography using silica gel (gradient hexane/ethylacetate) to obtain the title compound (2.13 g, 40% yield).

LC-MS (Method 1): Rₜ=0.92 min, MS (ESIpos): m/z=183 [M+H]⁺

¹H-NMR (400 MHz, DMSO-d6) δ [ppm]: 1.170 (6.09), 1.188 (12.55), 1.208 (6.57), 1.245 (7.14), 1.263 (16.00), 1.280 (7.23), 2.601 (1.10), 2.602 (1.08), 2.619 (3.24), 2.621 (3.35), 2.638 (3.29), 2.640 (3.34), 2.657 (1.02), 2.659 (1.03), 3.331 (8.78), 4.200 (1.95), 4.218 (6.25), 4.236 (6.29), 4.254 (1.95), 5.759 (0.98), 6.518 (4.92).

### Intermediate 14

### Ethyl 4-bromo-5-ethyl-1-methyl-1H-pyrazole-3-carboxylate

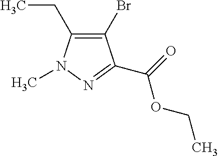

Ethyl-5-ethyl-1-methyl-1H-pyrazole-3-carboxylate (see intermediate 13, 2.10 g, 11.5 mmol) was dissolved in 15 mL of acetic acid. A solution of bromine in acetic acid (23 mL, 1.0 M, 23 mmol) was added dropwise and the reaction mixture was stirred for 18 hours at rt. The mixture was poured into ice water and aqueous sodium thiosulfate solution (10%) was added. The mixture was extracted with ethylacetate and the combined organic layers were washed with brine, dried using a water resistant filter and concentrated under reduced pressure to obtain 2.97 g of the title compound. The crude material was used without further purification in the next step.

LC-MS (Method 1): Rₜ=1.08 min, MS (ESIpos): m/z=261 [M+H]⁺

¹H-NMR (400 MHz, DMSO-d6) δ [ppm]: 1.077 (2.69), 1.096 (6.29), 1.115 (2.81), 1.260 (3.48), 1.278 (7.87), 1.295 (3.68), 1.907 (1.63), 2.518 (0.62), 2.523 (0.41), 2.673 (0.89), 2.692 (2.71), 2.711 (2.65), 2.730 (0.75), 3.894 (16.00), 4.231 (1.11), 4.249 (3.60), 4.266 (3.59), 4.284 (1.10).

### Intermediate 15

### (4-Bromo-5-ethyl-1-methyl-1H-pyrazol-3-yl)methanol

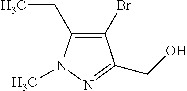

Ethyl 4-bromo-5-ethyl-1-methyl-1H-pyrazole-3-carboxylate (see intermediate 14, 2.97 g) was dissolved in 45 mL of THF and lithium borohydride (310 mg, 14.2 mmol) was added portionwise. This mixture was stirred for 20 hours at rt and for 22 hours at 60° C. Lithium borohydride (50 mg, 2.3 mmol) was added and stirring was continued for 24 hours at rt and 3 hours at 60° C. The reaction mixture was diluted with saturated aqueous chloride solution and extracted with ethylacetate. The combined organic layers were washed with brine, dried using a water resistant filter and concentrated under reduced pressure to obtain the title compound (2.18 g). The crude material was used without further purification in the next step.

LC-MS (Method 1): Rₜ=0.79 min, MS (ESIpos): m/z=219 [M+H]⁺

¹H-NMR (400 MHz, DMSO-d6) δ [ppm]: 1.068 (3.21), 1.087 (7.19), 1.105 (3.37), 2.518 (0.44), 2.609 (1.02), 2.628 (3.36), 2.647 (3.29), 2.666 (1.04), 3.761 (16.00), 4.287 (4.77), 4.301 (4.91), 4.941 (1.34), 4.955 (2.69), 4.969 (1.21).

### Intermediate 16

### Ethyl 3-ethyl-1H-pyrazole-5-carboxylate

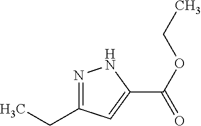

To a solution of ethyl-2,4-dioxohexanoate (CAS 13246-52-1, 942 g, 5.47 mol) in 3520 mL of ethanol, acetic acid (328.72 g, 5.47 mol, 313.07 mL, 1 eq) was added dropwise, and the resulting solution was stirred for 30 min at 0° C. Subsequently, hydrazine hydrate (279.63 g, 5.47 mol, 271.49 mL) was added dropwise, then the reaction mixture was allowed to warm to 20° C. and was then maintained at that temperature over a period of 16 h, before being evaporated under reduced pressure. The residue was diluted with water (1 L) and extracted with dichloromethane (500 mL) thrice. The combined organic layers were washed with brine (500 mL), dried over sodium sulfate, filtered, and concentrated under reduced pressure. The crude material was purified by flash silica gel chromatography, eluent of 0-50% ethyl acetate/petroleum ether, to give the title compound (1800 g, 38% yield, 98% purity) as a red oil.

### Intermediate 17

### Ethyl 3-ethyl-1-methyl-1H-pyrazole-5-carboxylate

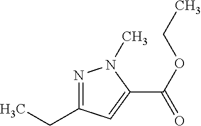

To a solution of ethyl 3-ethyl-1H-pyrazole-5-carboxylate (see intermediate 16, 400 g, 2.38 mol) in dichloromethane (400 mL) was added sodium carbonate (360 g, 3.40 mol, 1.43 eq). It was heated to 40° C. and then dimethylsulfate (420 g, 3.33 mol, 315.76 mL, 1.4 eq) was added. The reaction mixture was heated to 90° C. and stirred for 2 hours. The reaction was stopped by addition of water (500 mL) at 10° C., and the mixture was then extracted with ethylacetate (500 mL) thrice. The combined organic layers were washed with brine (200 mL), dried over sodium sulfate, filtered and concentrated under reduced pressure. The crude material was purified by flash silica gel chromatography, eluent of 0-5% ethyl acetate/petroleum ether, to give the title compound (226 g, 52% yield) as a light yellow oil.

¹H-NMR (CDCl3, 400 MHz) δ [ppm]: 6.63 (s, 1H), 4.32 (q, J=7.15 Hz, 2H), 4.11 (s, 3H), 2.63 (q, J=7.61 Hz, 2H), 1.36 (t, J=7.15 Hz, 3H), 1.21-1.25 (m, 3H)

### Intermediate 18

### Ethyl 4-bromo-3-ethyl-1-methyl-1H-pyrazole-5-carboxylate

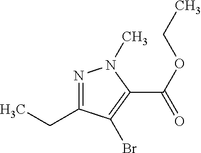

To a solution of ethyl 3-ethyl-1-methyl-1H-pyrazole-5-carboxylate (see intermediate 17, 250 g, 1.37 mol, 1 eq) in dichloromethane (3800 mL) was added bromine (658 g, 4.12 mol, 212.18 mL, 3 eq) dropwise at 0° C. The mixture was stirred at 10° C. for 3 hours. The reaction was stopped by the addition of an aqueous sodium hydrosulfite solution (1500 mL), and the mixture was then diluted with water (500 mL), and was extracted with dichloromethane (800 mL) thrice. The combined organic layers were washed with brine (800 mL), dried over sodium sulfate, filtered and concentrated under reduced pressure to give the title compound (300 g, 80% yield, 96% purity) as a yellow solid.

¹H NMR (MeOD, 400 MHz) δ [ppm]: 4.33-4.43 (m, 2H), 4.07 (s, 3H), 2.61 (q, J=7.65 Hz, 2H), 1.40 (t, J=7.15 Hz, 3H), 1.17-1.24 (m, 3H)

### Intermediate 19

### (4-Bromo-3-ethyl-1-methyl-1H-pyrazol-5-yl)methanol

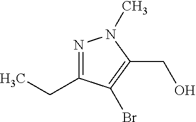

To a solution of ethyl 4-bromo-3-ethyl-1-methyl-1H-pyrazole-5-carboxylate (see intermediate 18, 200 g, 766 mmol, 1 eq) in tetrahydrofurane (2000 mL) was added lithium borohydride (83.4 g, 3.83 mol, 5 eq) slowly. The mixture was stirred at 60° C. for 3 hours. The reaction was stopped by addition of water (2000 mL) at 20° C., and the mixture was then extracted with ethyl acetate (1000 mL) thrice. The combined organic layers were washed with brine (800 mL), dried over sodium sulfate, filtered and concentrated under reduced pressure. The crude material was purified by triturating with petroleum ether (2000 mL) for three times to give the title compound (112 g, 66% yield, 99% purity) as a white solid.

¹H NMR (MeOD, 400 MHz) δ [ppm]: 4.60 (s, 2H), 3.86 (s, 3H), 2.58 (q, J=7.57 Hz, 2H), 1.20 (t, J=7.59 Hz, 3H)

### Intermediate 20

### Ethyl 4-bromo-1,3-dimethyl-1H-pyrazole-5-carboxylate

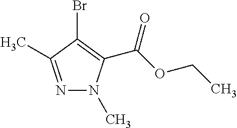

N-Bromosuccinimide (11.2 g, 99% purity, 62.4 mmol) was added to a solution of ethyl 1,3-dimethyl-1H-pyrazole-5-carboxylate (CAS: 5744-40-1, 5.00 g, 29.7 mmol) in 1,2-dichloroethane (100 mL) and the mixture was stirred for 4 hours at 65° C., 8 hours at 80° C. and finally at rt for 72 hours. For work-up, the mixture was diluted with ethyl acetate, washed with water thrice and the organic phase was filtered through a silicone filter and concentrated. The residue was purified by flash chromatography (Biotage SNAP cartridge silica 340 g, hexane/dichloromethane gradient, 0->100% dichloromethane) to give the title compound (6.69 g, 89% yield).

LC-MS (Method 2): Rₜ=1.15 min, MS (ESIpos): m/z=249 [M+H]⁺

¹H-NMR (400 MHz, DMSO-d6) δ [ppm]: 1.308 (4.21), 1.325 (8.89), 1.343 (4.18), 2.155 (14.47), 3.862 (1.45), 4.008 (16.00), 4.302 (1.34), 4.320 (4.19), 4.337 (4.07), 4.355 (1.24).

### Intermediate 21

### (4-Bromo-1,3-dimethyl-1H-pyrazol-5-yl)methanol

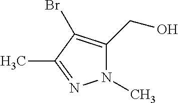

Lithium borohydride solution (23 mL, 1.0 M, 23 mmol, in THF) was added to a solution of ethyl 4-bromo-1,3-dimethyl-1H-pyrazole-5-carboxylate (see intermediate 20, 5.58 g, 22.6 mmol) in THF (200 mL), and the mixture was stirred for 48 hours at room temperature and 4 hours at 60° C. Lithium borohydride solution (11.5 mL, 1.0 M, 11.5 mmol) was added and stirring was continued at 60° C. overnight. The reaction was stopped by addition of saturated aqueous ammonium chloride solution, and the mixture was extracted with ethyl acetate. The organic phase was filtered through a silicone filter and concentrated to give the title compound (5.30 g).

LC-MS (Method 2): Rₜ=0.69 min; MS (ESIpos): m/z=207 [M+H]⁺

¹H-NMR (400 MHz, DMSO-d6) δ [ppm]: 2.084 (16.00), 2.518 (0.57), 3.331 (11.43), 4.422 (5.42), 4.436 (5.83), 5.309 (1.44), 5.322 (3.28), 5.336 (1.37), 6.552 (0.56).

### Intermediate 22

### Ethyl 7-[3-(hydroxymethyl)-1,5-dimethyl-1H-pyrazol-4-yl]-3-[3-(naphthalen-1-yloxy)propyl]-1H-indole-2-carboxylate

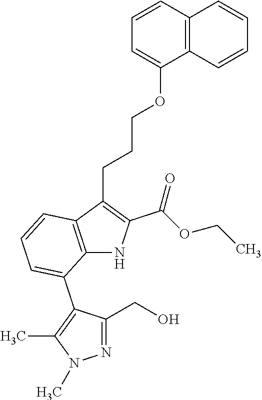 [SMILES: CCOC(=O)c1[nH]c2c(-c3c(CO)nn(C)c3C)cccc2c1CCCOc1cccc2ccccc12]

XPhos Pd G2 (see abbreviations; 483 mg, 613 μmol) was added to a degassed mixture of ethyl 3-[3-(naphthalen-1-yloxy)propyl]-7-(4,4,5,5-tetramethyl-1,3,2-dioxaborolan-2-yl)-1H-indole-2-carboxylate (see intermediate 1; 10.0 g, 20.0 mmol), (4-bromo-1,5-dimethyl-1H-pyrazol-3-yl)methanol (3.73 g, 18.2 mmol, see intermediate 12), aqueous potassium phosphate solution (73 mL, 0.50 M, 36 mmol) and THF (220 mL). The mixture was stirred for 2 h at 45° C. For work-up, ethyl acetate was added, the mixture was filtered through a pad of celite, eluted with ethyl acetate and the organic phase was washed with brine, filtered through a silicone filter and concentrated. The residue was purified by flash chromatography (Biotage SNAP cartridge silica 340 g, hexane/ethyl acetate gradient, 50%->100% ethyl acetate) to give the title compound (6.26 g, 63% yield).

LC-MS (Method 1): Rₜ=1.53 min, MS (ESIpos): m/z=498 [M+H]⁺

¹H-NMR (400 MHz, DMSO-d6) δ [ppm]: 1.155 (0.41), 1.173 (0.87), 1.190 (0.43), 1.256 (4.82), 1.273 (10.89), 1.291 (4.91), 1.988 (1.61), 2.164 (15.76), 2.205 (5.25), 2.213 (1.06), 2.231 (1.35), 2.250 (1.03), 2.518 (4.03), 2.523 (2.82), 3.355 (2.05), 3.373 (1.25), 3.726 (4.77), 3.802 (16.00), 4.199 (1.49), 4.214 (3.13), 4.222 (2.24), 4.229 (1.83), 4.240 (5.74), 4.249 (2.82), 4.258 (5.54), 4.275 (1.50), 4.286 (1.45), 4.300 (1.40), 4.947 (0.67), 5.705 (1.59), 6.907 (1.76), 6.925 (1.90), 7.060 (0.72), 7.077 (2.44), 7.090 (2.89), 7.096 (5.04), 7.108 (0.79), 7.373 (1.28), 7.394 (2.46), 7.413 (2.02), 7.450 (2.56), 7.471 (1.40), 7.492 (0.58), 7.505 (1.56), 7.509 (1.42), 7.514 (1.62), 7.521 (3.30), 7.529 (1.74), 7.533 (1.54), 7.538 (1.62), 7.551 (0.63), 7.656 (1.49), 7.662 (1.33), 7.674 (1.37), 7.679 (1.33), 7.861 (1.49), 7.868 (0.80), 7.879 (1.35), 7.884 (1.25), 8.230 (1.30), 8.236 (1.23), 8.254 (1.25), 11.324 (1.73).

### Intermediate 23

### (rac)-Ethyl 4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylate

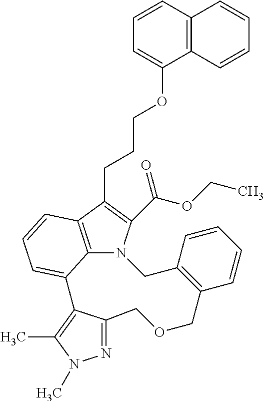 [SMILES: CCOC(=O)c1c(CCCOc2cccc3ccccc23)c2cccc3c2n1Cc1ccccc1COCc1nn(C)c(C)c1-3]

To a mixture of ethyl 7-[3-(hydroxymethyl)-1,5-dimethyl-1H-pyrazol-4-yl]-3-[3-(naphthalen-1-yloxy)propyl]-1H-indole-2-carboxylate (see intermediate 22, 200 mg, 402 μmol), acetonitrile (10 mL) and tetrahydrofurane (5 mL) cesium carbonate (655 mg, 2.01 mmol) was added and the mixture stirred for 10 minutes. After addition of 1,2-bis(bromomethyl)benzene (CAS 91-13-4, 123 mg, 95% purity, 442 μmol) the mixture was stirred 15 hours at ambient temperature followed by six hours at 60° C. and two hours at 80° C. After cooling the mixture was evaporated to dryness, and the residue was subjected to flash chromatography (Biotage SNAP cartridge silica, dichloromethane/acetone gradient, 0%->20% acetone) to give the title compound (100 mg).

LC-MS (Method 2): Rₜ=1.66 min, MS (ESIpos): m/z=601 [M+H]⁻

¹H-NMR (400 MHz, DMSO-d6) δ [ppm]: 1.143 (4.70), 1.161 (10.68), 1.178 (4.78), 1.232 (0.47), 1.952 (15.57), 2.084 (11.59), 2.104 (1.30), 2.121 (0.95), 2.518 (4.16), 2.523 (2.93), 3.069 (0.50), 3.085 (0.56), 3.103 (0.82), 3.202 (0.85), 3.219 (0.58), 3.237 (0.54), 3.810 (16.00), 3.887 (1.36), 3.912 (2.00), 4.008 (1.98), 4.034 (1.81), 4.045 (0.68), 4.060 (1.09), 4.076 (0.49), 4.090 (0.50), 4.105 (1.15), 4.117 (2.58), 4.130 (0.89), 4.138 (4.33), 4.149 (2.47), 4.155 (4.35), 4.173 (1.22), 4.371 (2.27), 4.404 (1.96), 5.140 (1.46), 5.177 (1.83), 5.401 (1.73), 5.438 (1.48), 5.759 (0.82), 6.783 (1.73), 6.801 (1.88), 6.988 (1.77), 6.991 (1.88), 7.006 (2.35), 7.009 (2.23), 7.072 (1.09), 7.076 (1.26), 7.090 (1.57), 7.095 (1.81), 7.100 (2.31), 7.105 (1.20), 7.109 (1.17), 7.120 (2.76), 7.123 (1.86), 7.128 (1.75), 7.138 (1.57), 7.146 (0.62), 7.150 (0.80), 7.165 (1.67), 7.169 (1.46), 7.182 (1.42), 7.186 (2.17), 7.191 (1.63), 7.204 (1.40), 7.209 (1.24), 7.223 (0.45), 7.354 (1.40), 7.374 (2.49), 7.393 (1.94), 7.442 (2.50), 7.463 (1.50), 7.471 (0.52), 7.476 (0.64), 7.489 (1.48), 7.493 (1.30), 7.503 (1.63), 7.509 (2.35), 7.513 (1.63), 7.523 (1.44), 7.527 (1.61), 7.540 (0.72), 7.544 (0.49), 7.722 (1.92), 7.725 (1.98), 7.742 (1.83), 7.744 (1.67), 7.850 (1.48), 7.856 (0.91), 7.869 (1.65), 7.874 (1.28), 8.172 (1.32), 8.176 (1.32), 8.194 (1.26), 8.196 (1.24).

### Intermediate 24

### (rac)-Ethyl 4,5-dimethyl-17-[3-(naphthalen-1-yloxy)propyl]-5,7,9,14-tetrahydropyrazolo[4′,3′:9,10]pyrido[3′,2′:3,4][1,6]oxazacycloundecino[8,7,6-hi]indole-16-carboxylate

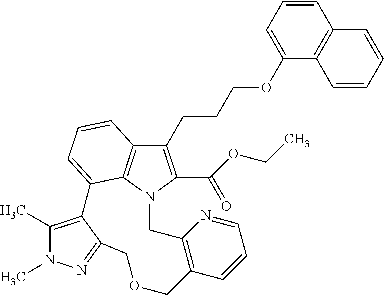 [SMILES: CCOC(=O)c1c(CCCOc2cccc3ccccc23)c2cccc3c2n1Cc1ncccc1COCc1nn(C)c(C)c1-3]

To a mixture of ethyl 7-[3-(hydroxymethyl)-1,5-dimethyl-1H-pyrazol-4-yl]-3-[3-(naphthalen-1-yloxy)propyl]-1H-indole-2-carboxylate (see intermediate 22, 200 mg, 402 μmol) in acetonitrile (5.5 mL) was added cesium carbonate (786 mg, 2.41 mmol), and the mixture was stirred for 10 minutes. 2,3-Bis(chloromethyl)pyridine hydrochloride (CAS 27221-49-4, 128 mg, 603 μmol) and sodium iodide (120 mg, 804 μmol) were added and the reaction mixture was stirred for 72 hours at 75° C. The mixture was poured into water and the mixture was extracted with ethyl acetate. The organic layer was dried over sodium sulfate, filtered and concentrated under reduced pressure. The crude product was purified by preparative HPLC (Method P3) to give the title compound (71 mg, 30% yield). In addition, ethyl 4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino-[8,7,6-hi]indole-16-carboxylate was isolated (49 mg, 20% yield, see next intermediate).

LC-MS (Method 1): Rₜ=1.53 min, MS (ESIpos): m/z=601 [M+H]⁺

¹H-NMR (400 MHz, DMSO-d6) δ [ppm]: 1.269 (3.61), 1.286 (8.05), 1.304 (3.74), 1.877 (11.94), 2.084 (16.00), 2.147 (0.79), 2.164 (1.30), 2.181 (0.86), 2.331 (1.33), 2.336 (0.60), 2.518 (7.00), 2.523 (4.72), 2.673 (1.30), 2.678 (0.57), 3.185 (0.48), 3.204 (0.89), 3.214 (0.51), 3.223 (0.54), 3.231 (0.86), 3.249 (0.54), 3.854 (11.91), 4.046 (1.24), 4.072 (1.71), 4.091 (0.79), 4.184 (0.79), 4.201 (0.48), 4.208 (0.63), 4.255 (0.67), 4.266 (1.52), 4.272 (0.86), 4.282 (1.62), 4.290 (2.63), 4.299 (1.58), 4.308 (1.55), 4.320 (1.90), 4.325 (1.58), 4.335 (0.57), 4.352 (0.51), 4.616 (1.65), 4.648 (1.39), 5.278 (2.15), 5.287 (2.06), 6.830 (1.27), 6.847 (1.39), 6.927 (1.36), 6.930 (1.43), 6.945 (1.87), 6.947 (1.71), 7.030 (1.58), 7.049 (1.84), 7.067 (1.20), 7.084 (1.20), 7.096 (1.24), 7.103 (1.24), 7.115 (1.27), 7.382 (0.95), 7.403 (1.90), 7.422 (1.65), 7.450 (2.06), 7.470 (1.01), 7.492 (0.48), 7.504 (1.27), 7.511 (1.74), 7.520 (2.76), 7.529 (3.23), 7.533 (2.06), 7.548 (1.62), 7.552 (1.43), 7.597 (1.43), 7.600 (1.55), 7.617 (1.39), 7.619 (1.30), 7.857 (1.14), 7.866 (0.60), 7.875 (0.86), 7.880 (0.98), 8.221 (1.01), 8.228 (0.86), 8.237 (0.48), 8.245 (0.95), 8.293 (1.49), 8.297 (1.49), 8.305 (1.43), 8.309 (1.30)

### Intermediate 25

### (rac)-Ethyl 4,5-dimethyl-17-[3-(naphthalen-1-yloxy)propyl]-5,7,9,14-tetrahydropyrazolo[4′,3′:9,10]pyrido[2′,3′:3,4][1,6]oxazacycloundecino[8,7,6-hi]indole-16-carboxylate

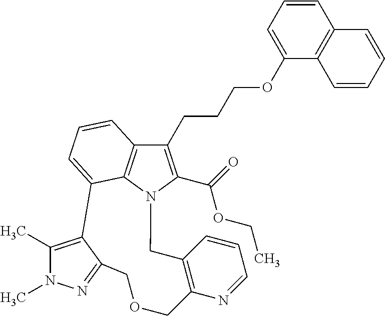 [SMILES: CCOC(=O)c1c(CCCOc2cccc3ccccc23)c2cccc3c2n1Cc1cccnc1COCc1nn(C)c(C)c1-3]

The title compound was isolated as side product in the preparation of intermediate 24.

LC-MS (Method 1): Rₜ=1.61 min, MS (ESIpos): m/z=601 [M+H]⁺

¹H-NMR (400 MHz, DMSO-d6) δ [ppm]: 1.133 (3.01), 1.151 (6.79), 1.168 (3.10), 1.255 (0.49), 1.273 (1.04), 1.291 (0.52), 1.954 (9.80), 2.084 (16.00), 2.095 (0.68), 2.113 (0.95), 2.129 (0.64), 2.164 (1.47), 2.331 (1.29), 2.336 (0.58), 2.518 (6.69), 2.523 (4.55), 2.539 (0.71), 2.673 (1.26), 2.678 (0.55), 3.138 (0.55), 3.223 (0.61), 3.240 (0.43), 3.257 (0.43), 3.794 (10.17), 3.801 (1.93), 3.973 (1.01), 3.998 (1.32), 4.058 (0.43), 4.066 (0.46), 4.083 (0.74), 4.099 (0.49), 4.117 (1.54), 4.134 (2.98), 4.152 (2.79), 4.160 (1.66), 4.169 (1.01), 4.177 (1.35), 4.184 (1.29), 4.209 (1.50), 4.239 (0.61), 4.257 (0.58), 4.402 (1.54), 4.434 (1.26), 5.227 (0.95), 5.264 (1.26), 5.424 (1.11), 5.461 (0.89), 6.799 (1.14), 6.817 (1.26), 7.015 (1.17), 7.018 (1.17), 7.032 (1.57), 7.035 (1.44), 7.096 (0.49), 7.117 (1.35), 7.137 (1.47), 7.155 (1.01), 7.233 (1.01), 7.245 (1.04), 7.252 (1.14), 7.264 (1.14), 7.358 (0.89), 7.379 (1.63), 7.398 (1.35), 7.443 (1.69), 7.464 (1.07), 7.476 (0.40), 7.479 (0.46), 7.493 (1.14), 7.497 (1.14), 7.500 (1.44), 7.505 (2.09), 7.511 (2.15), 7.517 (1.41), 7.524 (1.90), 7.529 (1.38), 7.542 (0.61), 7.546 (0.43), 7.743 (1.20), 7.747 (1.26), 7.764 (1.20), 7.767 (1.11), 7.852 (1.07), 7.859 (0.71), 7.870 (1.17), 7.875 (0.92), 8.177 (0.86), 8.182 (0.86), 8.201 (0.83), 8.327 (1.17), 8.330 (1.23), 8.338 (1.17), 8.342 (1.11)

### Intermediate 26

### (rac)-Ethyl 4,5-dimethyl-19-[3-(naphthalen-1-yloxy)propyl]-5,7,9,16-tetrahydroindolo[1′,7′:6,7,8]pyrazolo[4′,3′:9,10][1,6]oxazacycloundecino[3,4-b]quinoxaline-18-carboxylate

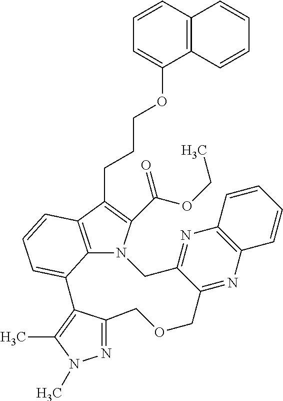 [SMILES: CCOC(=O)c1c(CCCOc2cccc3ccccc23)c2cccc3c2n1Cc1nc2ccccc2nc1COCc1nn(C)c(C)c1-3]

To a mixture of ethyl 7-[3-(hydroxymethyl)-1,5-dimethyl-1H-pyrazol-4-yl]-3-[3-(naphthalen-1-yloxy)propyl]-1H-indole-2-carboxylate (see intermediate 22, 200 mg, 402 μmol), acetonitrile (10 mL) and tetrahydrofurane (10 mL), cesium carbonate (655 mg, 2.01 mmol) was added and the mixture was stirred for 10 minutes. After addition of 2,3-bis(bromomethyl)quinoxaline (CAS 3138-86-1, 143 mg, 98% purity, 442 μmol) the mixture was stirred 23 hours at ambient temperature followed by four hours at 80° C. After cooling, the mixture was evaporated to dryness and the residue was subjected to flash chromatography (Biotage SNAP cartridge silica, dichloromethane/ethanol gradient, 0%->10% ethanol) to give the title compound (53 mg).

LC-MS (Method 2): Rₜ=1.65 min, MS (ESIpos): m/z=653 [M+H]⁺

¹H-NMR (400 MHz, DMSO-d6) δ [ppm]: 0.797 (0.76), 0.803 (0.44), 0.814 (1.14), 0.821 (0.93), 0.828 (0.54), 0.840 (0.54), 0.850 (0.87), 0.870 (0.54), 0.884 (1.96), 0.901 (4.03), 0.920 (2.18), 0.991 (1.25), 1.009 (1.31), 1.024 (0.44), 1.035 (0.82), 1.041 (0.93), 1.053 (1.47), 1.070 (0.87), 1.088 (0.60), 1.136 (0.49), 1.231 (5.06), 1.273 (0.54), 1.305 (0.44), 1.321 (4.95), 1.339 (10.50), 1.357 (4.84), 1.740 (1.03), 1.748 (0.98), 1.756 (2.88), 1.765 (0.98), 1.773 (0.98), 1.916 (15.56), 1.987 (0.49), 2.056 (0.65), 2.065 (5.66), 2.084 (4.14), 2.091 (1.09), 2.127 (0.87), 2.145 (1.41), 2.158 (1.69), 2.178 (1.31), 2.195 (0.71), 2.223 (1.90), 2.336 (1.14), 2.401 (1.36), 2.407 (0.82), 2.420 (1.63), 2.422 (1.63), 2.437 (1.31), 2.443 (0.76), 2.459 (0.76), 2.464 (1.03), 2.518 (13.22), 2.522 (8.93), 2.534 (1.09), 2.538 (0.93), 2.544 (0.65), 2.549 (0.60), 2.565 (0.87), 2.678 (1.14), 2.777 (0.44), 3.230 (1.31), 3.249 (2.56), 3.268 (1.31), 3.582 (1.09), 3.592 (0.98), 3.598 (2.50), 3.604 (0.93), 3.615 (1.03), 3.642 (0.71), 3.796 (2.23), 3.825 (0.82), 3.871 (16.00), 4.039 (0.44), 4.055 (0.93), 4.063 (1.14), 4.079 (1.14), 4.095 (0.54), 4.150 (0.98), 4.166 (1.41), 4.183 (0.87), 4.190 (1.03), 4.207 (0.60), 4.240 (1.52), 4.258 (2.34), 4.275 (1.25), 4.301 (0.44), 4.318 (2.39), 4.328 (1.36), 4.342 (2.45), 4.355 (1.74), 4.373 (3.43), 4.390 (0.54), 4.405 (2.23), 4.431 (0.49), 4.449 (1.58), 4.458 (0.44), 4.466 (1.47), 4.476 (1.09), 4.485 (0.54), 4.494 (1.09), 4.685 (1.85), 4.708 (1.63), 4.744 (2.12), 4.776 (1.85), 5.421 (0.76), 5.459 (2.50), 5.482 (2.56), 5.520 (0.93), 5.759 (1.74), 6.735 (1.90), 6.754 (2.07), 6.872 (0.44), 6.970 (0.49), 6.993 (1.69), 6.996 (1.80), 7.010 (2.78), 7.013 (2.61), 7.036 (0.44), 7.053 (2.45), 7.072 (2.88), 7.091 (1.96), 7.240 (0.44), 7.305 (1.80), 7.325 (2.94), 7.345 (2.29), 7.364 (0.71), 7.376 (0.71), 7.396 (0.76), 7.428 (3.32), 7.442 (1.41), 7.449 (2.72), 7.477 (1.20), 7.482 (1.25), 7.494 (2.56), 7.498 (2.23), 7.503 (2.72), 7.511 (4.41), 7.518 (2.61), 7.522 (2.45), 7.527 (2.39), 7.539 (1.20), 7.544 (0.87), 7.602 (2.23), 7.605 (2.29), 7.622 (2.01), 7.625 (1.96), 7.685 (0.44), 7.706 (0.38), 7.738 (0.71), 7.742 (0.87), 7.755 (1.52), 7.759 (1.58), 7.774 (2.12), 7.780 (2.45), 7.797 (1.90), 7.801 (2.18), 7.814 (1.36), 7.818 (1.25), 7.838 (2.88), 7.841 (2.67), 7.844 (2.83), 7.858 (2.23), 7.863 (2.83), 7.869 (2.01), 7.931 (1.90), 7.934 (2.23), 7.949 (1.47), 7.951 (1.47), 7.954 (1.63), 7.964 (0.60), 7.970 (0.87), 7.981 (2.23), 7.985 (2.39), 7.987 (2.29), 7.995 (3.54), 8.005 (2.83), 8.010 (2.29), 8.021 (0.76), 8.027 (0.44), 8.136 (0.44), 8.190 (1.47), 8.196 (1.41), 8.214 (1.58), 8.228 (1.90), 8.234 (1.41), 8.238 (1.36), 8.247 (1.25), 8.252 (1.63), 8.307 (1.69), 8.312 (1.36), 8.319 (1.03), 8.322 (1.14), 8.326 (1.20), 8.331 (1.41), 10.025 (6.80).

### Intermediate 27

### Ethyl 1-[2-(bromomethyl)benzyl]-7-[3-(hydroxymethyl)-1,5-dimethyl-1H-pyrazol-4-yl]-3-[3-(naphthalen-1-yloxy)propyl]-1H-indole-2-carboxylate

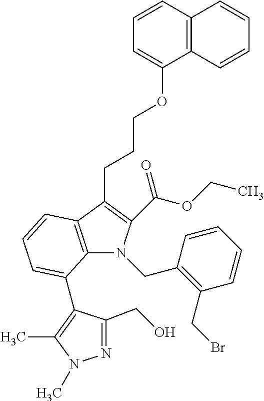 [SMILES: CCOC(=O)c1c(CCCOc2cccc3ccccc23)c2cccc(-c3c(CO)nn(C)c3C)c2n1Cc1ccccc1CBr]

To a mixture of ethyl 7-[3-(hydroxymethyl)-1,5-dimethyl-1H-pyrazol-4-yl]-3-[3-(naphthalen-1-yloxy)propyl]-1H-indole-2-carboxylate (see intermediate 22, 18.1 g, 36.4 mmol), acetonitrile (600 mL) and tetrahydrofurane (400 mL), cesium carbonate (59.3 g, 182 mmol) was added and the mixture was stirred for 10 minutes. After addition of 1,2-bis(bromomethyl)benzene (CAS 91-13-4, 12.1 g, 95% purity, 43.7 mmol) the mixture was stirred 2 hours and 15 minutes at ambient temperature. After filtration over a bed of celite the filtrate was evaporated to dryness to yield the crude title compound (27.8 g).

LC-MS (Method 2): Rₜ=1.63 min, MS (ESIpos): m/z=681 [M+H]⁺

### Intermediate 28

### Ethyl 1-(2-{[bis(tert-butoxycarbonyl)amino]methyl}benzyl)-7-[3-(hydroxymethyl)-1,5-dimethyl-1H-pyrazol-4-yl]-3-[3-(naphthalen-1-yloxy)propyl]-1H-indole-2-carboxylate

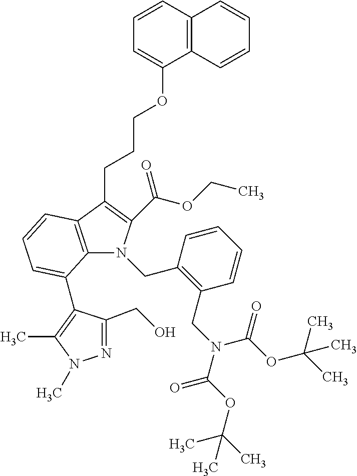 [SMILES: CCOC(=O)c1c(CCCOc2cccc3ccccc23)c2cccc(-c3c(CO)nn(C)c3C)c2n1Cc1ccccc1CN(C(=O)OC(C)(C)C)C(=O)OC(C)(C)C]

To a solution of di-tert-butyl imidodicarbonate (CAS 51779-32-9, 16.7 g, 95% purity, 72.9 mmol) in DMF (300 mL) sodium hydride (2.19 g, 60% purity, 54.6 mmol) was added in portions and the mixture was stirred for one hour at ambient temperature. Then a solution of ethyl 1-[2-(bromomethyl)benzyl]-7-[3-(hydroxymethyl)-1,5-dimethyl-1H-pyrazol-4-yl]-3-[3-(naphthalen-1-yloxy)propyl]-1H-indole-2-carboxylate (see intermediate 27, 24.8 g, 36.4 mmol) in DMF (300 mL) was added and the resulting mixture was stirred for 15 hours at ambient temperature, followed by stirring for an additional hour at 50° C. After cooling, the reaction mixture was poured into ice water and the organic matter was extracted with ethyl acetate. Then the organic phase was washed with a saturated aqueous sodium chloride solution, dried over sodium sulfate and filtered. The filtrate was evaporated and the residue was subjected to flash chromatography (Biotage SNAP cartridge silica, dichloromethane/acetone gradient, 0%->100% acetone) to give the title compound and mixed fractions, which were subjected to a second flash chromatography (Biotage SNAP cartridge silica, dichloromethane/acetone gradient, 0%->30% acetone). Overall 14.62 g of the title compound were obtained.

LC-MS (Method 2): Rₜ=1.80 min, MS (ESIpos): m/z=818 [M+H]⁺

¹H-NMR (400 MHz, DMSO-d6) δ [ppm]: 1.129 (0.84), 1.147 (1.87), 1.164 (0.87), 1.333 (0.73), 1.386 (16.00), 2.084 (1.66), 2.286 (0.22), 2.305 (0.17), 2.518 (0.47), 2.523 (0.33), 3.368 (0.19), 3.389 (0.27), 3.406 (0.18), 3.644 (2.20), 4.086 (0.16), 4.127 (0.39), 4.136 (0.34), 4.148 (0.39), 4.154 (0.37), 4.166 (0.33), 4.189 (0.32), 4.229 (0.17), 4.259 (0.23), 4.275 (0.47), 4.290 (0.23), 4.606 (0.19), 4.619 (0.41), 4.632 (0.18), 5.518 (0.28), 5.538 (0.22), 5.548 (0.32), 5.568 (0.26), 6.866 (0.25), 6.903 (0.31), 6.906 (0.32), 6.921 (0.36), 6.924 (0.35), 6.937 (0.31), 6.956 (0.47), 6.977 (0.31), 7.056 (0.18), 7.079 (0.47), 7.097 (0.40), 7.099 (0.42), 7.117 (0.28), 7.387 (0.24), 7.408 (0.45), 7.427 (0.37), 7.464 (0.46), 7.485 (0.25), 7.522 (0.42), 7.526 (0.30), 7.532 (0.35), 7.535 (0.38), 7.540 (0.32), 7.546 (0.44), 7.808 (0.32), 7.811 (0.34), 7.829 (0.31), 7.832 (0.30), 7.870 (0.26), 7.874 (0.17), 7.882 (0.24), 7.887 (0.17), 7.893 (0.22), 8.262 (0.23), 8.274 (0.19), 8.287 (0.21).

### Intermediate 29

### Ethyl 1-(2-{[bis(tert-butoxycarbonyl)amino]methyl}benzyl)-7-[3-(bromomethyl)-1,5-dimethyl-1H-pyrazol-4-yl]-3-[3-(naphthalen-1-yloxy)propyl]-1H-indole-2-carboxylate

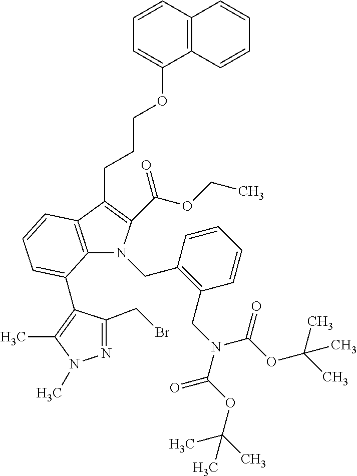 [SMILES: CCOC(=O)c1c(CCCOc2cccc3ccccc23)c2cccc(-c3c(CBr)nn(C)c3C)c2n1Cc1ccccc1CN(C(=O)OC(C)(C)C)C(=O)OC(C)(C)C]

A solution of ethyl-1-(2-{[bis(tert-butoxycarbonyl)amino]methyl}benzyl)-7-[3-(hydroxymethyl)-1,5-dimethyl-1H-pyrazol-4-yl]-3-[3-(naphthalen-1-yloxy)propyl]-1H-indole-2-carboxylate (see intermediate 28, 14.6 g, 17.9 mmol) in dichloromethane (330 mL) was cooled to 0° C. Then, triphenylphosphane (10.3 g, 39.3 mmol) was added and the mixture was stirred at 0° C. for 10 minutes before tetrabromomethane (10.7 g, 32.2 mmol) was added. After stirring at ambient temperature for 75 minutes, the reaction mixture was evaporated and the residue was subjected to flash chromatography (Biotage SNAP cartridge silica, hexane/ethyl acetate gradient, 0%->75% ethyl acetate) to give the title compound (13 g).

LC-MS (Method 1): Rₜ=1.92 min, MS (ESIpos): m/z=880 [M+H]⁺

¹H-NMR (400 MHz, DMSO-d6) δ [ppm]: 1.131 (0.81), 1.137 (0.18), 1.148 (1.81), 1.154 (0.42), 1.161 (0.44), 1.166 (0.86), 1.172 (0.60), 1.178 (0.18), 1.190 (0.28), 1.382 (16.00), 1.390 (3.67), 1.400 (1.03), 1.510 (0.63), 1.987 (0.93), 2.283 (0.21), 2.302 (0.27), 2.318 (0.24), 2.327 (0.21), 2.518 (0.55), 2.523 (0.38), 3.382 (0.25), 3.401 (0.37), 3.419 (0.23), 3.719 (2.18), 3.755 (0.38), 4.017 (0.21), 4.035 (0.21), 4.089 (0.18), 4.130 (0.54), 4.149 (0.72), 4.166 (0.73), 4.176 (0.39), 4.183 (0.36), 4.216 (0.16), 4.265 (0.28), 4.280 (0.54), 4.295 (0.27), 5.421 (0.17), 5.448 (0.20), 5.522 (0.32), 5.541 (0.27), 6.873 (0.28), 6.891 (0.19), 6.933 (0.35), 6.950 (0.39), 6.961 (0.37), 6.963 (0.37), 6.979 (0.40), 6.981 (0.38), 6.995 (0.23), 7.014 (0.35), 7.074 (0.19), 7.092 (0.30), 7.111 (0.16), 7.139 (0.35), 7.157 (0.40), 7.159 (0.40), 7.177 (0.28), 7.384 (0.24), 7.404 (0.47), 7.423 (0.38), 7.463 (0.49), 7.483 (0.27), 7.512 (0.31), 7.517 (0.57), 7.527 (0.63), 7.536 (0.60), 7.541 (0.39), 7.867 (0.29), 7.871 (0.24), 7.877 (0.52), 7.891 (0.31), 7.898 (0.41), 7.900 (0.43), 8.243 (0.24), 8.250 (0.21), 8.267 (0.24).

### Intermediate 30

### Ethyl 1-[2-(aminomethyl)benzyl]-7-[3-(bromomethyl)-1,5-dimethyl-1H-pyrazol-4-yl]-3-[3-(naphthalen-1-yloxy)propyl]-1H-indole-2-carboxylate-hydrochloric Acid Salt

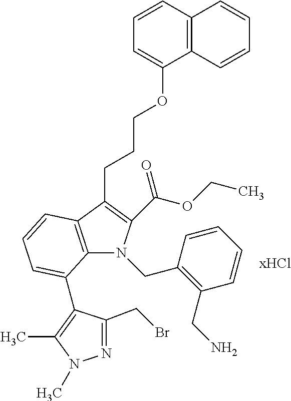 [SMILES: CCOC(=O)c1c(CCCOc2cccc3ccccc23)c2cccc(-c3c(CBr)nn(C)c3C)c2n1Cc1ccccc1CN]

To a solution of ethyl 1-(2-{[bis(tert-butoxycarbonyl)amino]methyl}benzyl)-7-[3-(bromomethyl)-1,5-dimethyl-1H-pyrazol-4-yl]-3-[3-(naphthalen-1-yloxy)propyl]-1H-indole-2-carboxylate (see intermediate 29, 13.3 g, 15.1 mmol) in ethanol (250 mL), a solution of hydrochloric acid in 1,4-dioxane (47 mL, 4.0 M, 190 mmol) was added at ambient temperature and the resulting reaction mixture was stirred at 50° C. for 90 minutes. Then, the reaction mixture was evaporated to dryness to give the crude title compound (11.97 g), which was further used without purification and analytics.

### Intermediate 31

### (rac)-Ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate

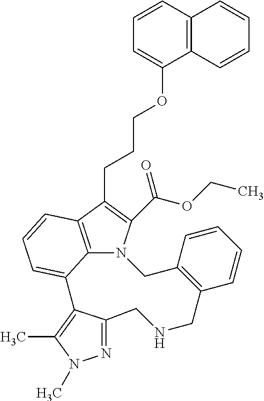 [SMILES: CCOC(=O)c1c(CCCOc2cccc3ccccc23)c2cccc3c2n1Cc1ccccc1CNCc1nn(C)c(C)c1-3]

To a solution of crude ethyl 1-[2-(aminomethyl)benzyl]-7-[3-(bromomethyl)-1,5-dimethyl-1H-pyrazol-4-yl]-3-[3-(naphthalen-1-yloxy)propyl]-1H-indole-2-carboxylate-hydrochloric acid salt (see intermediate 30, 12.5 g) in acetonitrile (570 mL), cesium carbonate (27.0 g, 82.8 mmol) was added and the reaction mixture was stirred for 15 hours at 50° C. Then, the reaction mixture was evaporated to dryness and subjected to flash chromatography (Biotage SNAP cartridge silica, dichloromethane/ethanol gradient, 0%->40% ethanol) to give the title compound and mixed fractions, which were subjected to a second flash chromatography (Biotage SNAP cartridge silica, dichloromethane/ethanol gradient, 0%->40% ethanol). Overall 4.94 g of the title compound were obtained.

LC-MS (Method 2): Rₜ=1.61 min, MS (ESIpos): m/z=600

¹H-NMR (400 MHz, DMSO-d6) δ [ppm]: 1.054 (0.50), 1.210 (5.08), 1.228 (11.69), 1.246 (5.29), 1.811 (15.27), 2.171 (1.03), 2.188 (1.53), 2.204 (1.08), 2.332 (0.55), 2.518 (2.89), 2.523 (2.06), 2.673 (0.53), 3.188 (0.55), 3.204 (0.66), 3.222 (1.02), 3.247 (1.01), 3.264 (0.70), 3.280 (1.91), 3.298 (2.14), 3.356 (1.68), 3.387 (1.02), 3.610 (1.49), 3.645 (1.25), 3.798 (16.00), 4.105 (0.70), 4.114 (0.70), 4.121 (0.55), 4.130 (1.19), 4.145 (0.56), 4.154 (0.58), 4.170 (1.18), 4.179 (0.57), 4.185 (0.71), 4.194 (0.70), 4.216 (1.19), 4.234 (3.27), 4.251 (2.97), 4.268 (0.97), 5.281 (1.06), 5.319 (1.94), 5.390 (1.88), 5.428 (1.14), 5.759 (8.75), 6.811 (1.25), 6.831 (3.13), 6.851 (2.07), 6.908 (1.88), 6.911 (2.06), 6.926 (2.36), 6.929 (2.35), 6.999 (0.67), 7.003 (0.70), 7.017 (1.34), 7.021 (1.40), 7.036 (0.94), 7.040 (1.01), 7.048 (2.20), 7.062 (0.99), 7.066 (2.58), 7.068 (2.65), 7.080 (1.76), 7.083 (2.00), 7.086 (2.12), 7.098 (1.13), 7.101 (1.11), 7.112 (2.02), 7.116 (1.98), 7.131 (0.92), 7.135 (0.77), 7.367 (1.45), 7.388 (2.68), 7.407 (2.20), 7.451 (2.76), 7.471 (1.61), 7.486 (0.50), 7.490 (0.70), 7.503 (1.74), 7.508 (1.58), 7.512 (1.86), 7.520 (3.75), 7.527 (1.95), 7.532 (1.73), 7.536 (1.89), 7.549 (0.72), 7.553 (0.46), 7.692 (2.00), 7.694 (2.12), 7.712 (1.90), 7.715 (1.86), 7.859 (1.60), 7.867 (0.91), 7.877 (1.49), 7.882 (1.37), 8.221 (1.43), 8.226 (1.32), 8.238 (0.75), 8.243 (1.27), 8.245 (1.35).

### Intermediate 32

### (rac)-Ethyl 4,5,8-trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate

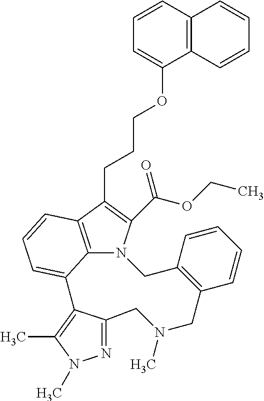 [SMILES: CCOC(=O)c1c(CCCOc2cccc3ccccc23)c2cccc3c2n1Cc1ccccc1CN(C)Cc1nn(C)c(C)c1-3]

A mixture of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 150 mg, 251 μmol) and formaldehyde (37.6 mg, 1.25 mmol) in methanol (5.0 mL) was stirred for 30 minutes at ambient temperature. Then sodium cyano borohydride (63.0 mg, 1.00 mmol) was added and stirring was continued for one hour. After addition of an aqueous sodium hydroxide solution (2 mL, 1.0 M, 2 mmol), followed by stirring for 5 minutes, the reaction mixture was partioned between water and dichloromethane. The organic phase then was filtered through a hydrophobic filter, the filtrate was evaporated and subjected to flash chromatography (Biotage SNAP cartridge silica, dichloromethane/ethanol gradient, 0.5%->10% ethanol) to give the title compound (118 mg).

LC-MS (Method 2): Rₜ=1.73 min, MS (ESIpos): m/z=613 [M+H]⁺

¹H-NMR (400 MHz, DMSO-d6) δ [ppm]: 0.884 (0.54), 0.902 (1.16), 0.920 (0.59), 1.035 (3.31), 1.052 (6.01), 1.070 (3.51), 1.207 (4.83), 1.225 (10.95), 1.243 (5.19), 1.850 (15.00), 2.066 (1.81), 2.085 (10.83), 2.111 (1.75), 2.128 (1.13), 2.327 (1.31), 2.331 (0.93), 2.336 (0.41), 2.420 (0.45), 2.438 (0.68), 2.518 (4.53), 2.523 (3.26), 2.669 (1.36), 2.673 (0.95), 2.678 (0.43), 2.894 (0.82), 2.926 (1.00), 3.088 (0.66), 3.103 (0.61), 3.122 (1.02), 3.143 (1.52), 3.176 (1.04), 3.207 (0.45), 3.226 (0.95), 3.242 (0.63), 3.259 (0.70), 3.361 (2.15), 3.405 (0.54), 3.417 (0.61), 3.423 (1.43), 3.435 (1.54), 3.440 (1.63), 3.452 (3.01), 3.470 (0.59), 3.488 (1.29), 3.783 (16.00), 4.042 (0.70), 4.051 (0.66), 4.066 (1.13), 4.082 (0.48), 4.098 (0.52), 4.113 (1.13), 4.129 (0.61), 4.137 (0.66), 4.207 (1.29), 4.225 (4.19), 4.243 (4.10), 4.260 (1.20), 4.344 (1.13), 4.356 (2.18), 4.369 (1.04), 5.166 (1.36), 5.203 (1.81), 5.373 (1.84), 5.410 (1.50), 5.760 (3.58), 6.783 (1.86), 6.801 (1.97), 6.936 (1.29), 6.942 (2.99), 6.944 (2.52), 6.959 (3.35), 6.962 (2.47), 7.055 (2.02), 7.067 (0.93), 7.075 (2.97), 7.083 (1.63), 7.093 (2.81), 7.097 (2.47), 7.105 (2.49), 7.112 (3.51), 7.121 (1.29), 7.127 (0.88), 7.357 (1.36), 7.377 (2.56), 7.396 (2.04), 7.444 (2.65), 7.465 (1.56), 7.478 (0.48), 7.482 (0.63), 7.495 (1.59), 7.499 (1.43), 7.507 (1.65), 7.513 (3.33), 7.519 (1.63), 7.526 (1.45), 7.530 (1.61), 7.543 (0.66), 7.547 (0.43), 7.650 (1.95), 7.653 (2.02), 7.670 (1.86), 7.672 (1.72), 7.853 (1.59), 7.860 (0.86), 7.871 (1.59), 7.877 (1.29), 8.192 (1.38), 8.198 (1.31), 8.217 (1.27).

### Intermediate 33

### (rac)-Ethyl 4,5-dimethyl-17-[3-(naphthalen-1-yloxy)propyl]-8-[(3,4,5-trimethoxybenzyl)carbamoyl]-7,8,9,14-tetrahydro-5H-indolo[1,7-bc]pyrazolo[4,3-e][2,8]benzodiazacycloundecine-16-carboxylate

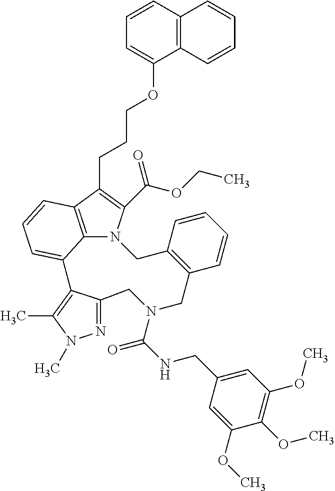

To a solution of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 150 mg, 251 μmol) in dichloromethane (1.6 mL), 5-(isocyanatomethyl)-1,2,3-trimethoxybenzene (CAS 351003-01-5, 55.9 mg, 251 μmol) and N,N-diisopropylethylamine (170 μl, 1.0 mmol) were added. After 17 hours stirring at ambient temperature, another portion of 5-(isocyanatomethyl)-1,2,3-trimethoxybenzene (11.2 mg, 50 μmol) was added and stirring was continued at 50° C. for one hour. After removal of all volatiles the residue was subjected to flash chromatography (Biotage SNAP cartridge silica, dichloromethane/ethanol gradient, 0%->10% ethanol) to give the title compound (157 mg).

LC-MS (Method 2): Rₜ=1.65 min, MS (ESIpos): m/z=823 [M+H]⁺

¹H-NMR (400 MHz, DMSO-d6) δ [ppm]: 1.053 (0.62), 1.219 (1.66), 1.237 (3.62), 1.255 (1.70), 1.739 (3.91), 2.294 (0.45), 2.305 (0.46), 2.322 (0.67), 2.327 (0.71), 2.331 (0.49), 2.518 (2.00), 2.523 (1.36), 2.669 (0.53), 3.351 (0.77), 3.369 (0.67), 3.628 (11.05), 3.685 (0.58), 3.724 (0.44), 3.754 (16.00), 3.788 (5.70), 4.216 (0.83), 4.227 (1.17), 4.234 (1.11), 4.245 (1.16), 4.251 (1.38), 4.264 (1.14), 5.413 (0.70), 5.449 (0.55), 5.760 (4.87), 6.475 (0.41), 6.495 (0.43), 6.631 (3.94), 6.808 (0.80), 6.811 (0.81), 6.826 (0.93), 6.829 (0.88), 6.905 (0.80), 6.917 (0.40), 6.923 (0.91), 6.932 (0.62), 7.024 (0.44), 7.029 (0.83), 7.042 (0.77), 7.049 (1.00), 7.059 (0.40), 7.067 (0.65), 7.205 (0.76), 7.222 (0.60), 7.380 (0.57), 7.400 (1.06), 7.420 (0.85), 7.460 (1.14), 7.481 (0.64), 7.511 (0.68), 7.516 (0.65), 7.519 (0.78), 7.528 (1.48), 7.535 (0.82), 7.539 (0.71), 7.543 (0.72), 7.732 (0.80), 7.734 (0.81), 7.752 (0.76), 7.754 (0.72), 7.867 (0.66), 7.885 (0.59), 7.890 (0.56), 8.255 (0.58), 8.260 (0.53), 8.278 (0.53).

### Intermediate 34

### (rac)-Ethyl 4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate

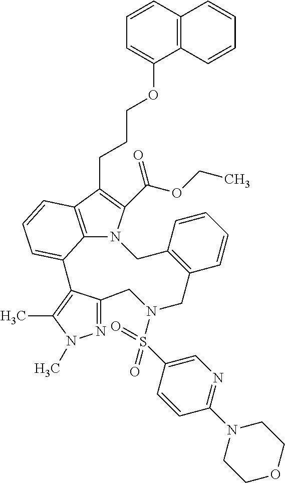 [SMILES: CCOC(=O)c1c(CCCOc2cccc3ccccc23)c2cccc3c2n1Cc1ccccc1CN(S(=O)(=O)c1ccc(N2CCOCC2)nc1)Cc1nn(C)c(C)c1-3]

To a solution of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 150 mg, 251 μmol) in acetonitrile (4.0 mL), 6-(morpholin-4-yl)pyridine-3-sulfonyl chloride (CAS 337508-68-6, 79.0 mg, 301 μmol) and N,N-diisopropylethylamine (87 μl, 500 μmol) were added. The mixture was then stirred for 17 hours at ambient temperature. After removal of all volatiles, the residue was subjected to flash chromatography (Biotage SNAP cartridge silica, dichloromethane/ethanol gradient, 0%->10% ethanol) to give the title compound (185 mg).

LC-MS (Method 2): Rₜ=1.68 min, MS (ESIpos): m/z=826 [M+H]⁺

¹H-NMR (400 MHz, DMSO-d6) δ [ppm]: 0.884 (1.05), 0.902 (2.28), 0.920 (1.10), 1.218 (4.51), 1.236 (9.99), 1.253 (4.64), 1.640 (11.15), 2.066 (2.83), 2.084 (1.68), 2.266 (1.15), 2.274 (1.15), 2.331 (1.13), 2.336 (0.52), 2.419 (0.71), 2.438 (0.79), 2.456 (0.81), 2.518 (5.53), 2.523 (3.99), 2.678 (0.50), 3.166 (0.45), 3.293 (0.39), 3.308 (0.76), 3.326 (1.60), 3.346 (1.70), 3.364 (1.13), 3.450 (2.23), 3.488 (2.02), 3.551 (1.44), 3.592 (16.00), 3.642 (4.62), 3.655 (4.38), 3.680 (0.76), 3.697 (4.80), 3.710 (4.88), 3.721 (2.44), 4.165 (0.63), 4.173 (0.76), 4.189 (1.26), 4.203 (1.29), 4.215 (2.26), 4.224 (1.34), 4.232 (1.50), 4.242 (2.65), 4.255 (2.60), 4.260 (2.33), 4.273 (1.76), 4.282 (0.63), 4.291 (0.52), 4.299 (0.45), 4.646 (1.31), 4.684 (1.23), 5.091 (1.29), 5.132 (1.68), 5.392 (1.23), 5.434 (1.02), 5.760 (11.10), 6.445 (0.94), 6.464 (0.97), 6.801 (1.91), 6.803 (1.94), 6.819 (2.23), 6.821 (2.10), 6.874 (1.94), 6.892 (2.10), 6.936 (1.00), 6.943 (2.02), 6.953 (1.73), 6.966 (2.15), 7.004 (1.99), 7.024 (2.41), 7.042 (1.60), 7.094 (1.00), 7.113 (1.73), 7.132 (0.89), 7.363 (1.65), 7.374 (1.84), 7.381 (1.50), 7.395 (2.89), 7.414 (2.31), 7.457 (2.96), 7.478 (1.70), 7.493 (0.45), 7.497 (0.68), 7.510 (1.78), 7.514 (1.68), 7.518 (2.05), 7.526 (3.93), 7.534 (2.12), 7.538 (1.78), 7.542 (1.94), 7.555 (0.71), 7.559 (0.45), 7.703 (2.18), 7.705 (2.20), 7.723 (2.05), 7.726 (1.97), 7.865 (1.73), 7.873 (0.89), 7.883 (1.50), 7.889 (1.44), 7.953 (1.05), 7.959 (1.08), 7.976 (1.00), 7.981 (0.97), 8.247 (1.50), 8.253 (1.36), 8.263 (0.73), 8.271 (1.36), 8.511 (1.89), 8.516 (1.84).

### Intermediate 35

### (rac)-Ethyl 4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylate

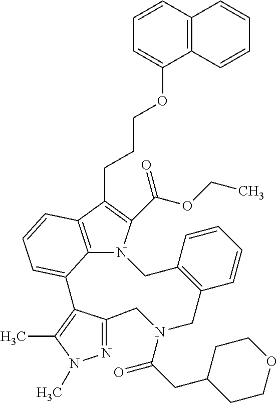 [SMILES: CCOC(=O)c1c(CCCOc2cccc3ccccc23)c2cccc3c2n1Cc1ccccc1CN(C(=O)CC1CCOCC1)Cc1nn(C)c(C)c1-3]

A mixture of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 150 mg, 251 μmol), (1H-benzotriazol-1-yloxy)(tripyrrolidin-1-yl)phosphonium hexafluorophosphate (143 mg, 276 μmol), tetrahydro-2H-pyran-4-ylacetic acid (CAS 85064-61-5, 39.7 mg, 276 μmol) and N,N-diisopropylethylamine (87 μl, 500 μmol) in DMF (3 mL) was stirred at ambient temperature for 90 minutes. After removal of all volatiles, the residue was subjected to flash chromatography (Biotage SNAP cartridge silica, dichloromethane/ethanol gradient, 0%->10% ethanol) to give the title compound (177 mg).

LC-MS (Method 2): Rₜ=1.70 min, MS (ESIpos): m/z=726 [M+H]⁺

¹H-NMR (400 MHz, DMSO-d6) δ [ppm]: 0.884 (1.91), 0.902 (3.95), 0.920 (1.91), 0.992 (0.60), 1.009 (0.64), 1.035 (7.16), 1.052 (16.00), 1.070 (8.00), 1.165 (1.17), 1.185 (1.27), 1.196 (1.17), 1.220 (3.92), 1.237 (7.67), 1.255 (3.51), 1.564 (0.90), 1.598 (0.84), 1.626 (0.94), 1.658 (0.80), 1.726 (9.47), 1.791 (0.44), 1.985 (0.67), 2.065 (5.29), 2.084 (1.07), 2.092 (0.50), 2.308 (1.64), 2.322 (2.91), 2.327 (3.21), 2.331 (2.48), 2.336 (1.74), 2.388 (1.10), 2.402 (1.07), 2.420 (1.54), 2.438 (1.67), 2.456 (1.37), 2.518 (7.67), 2.523 (4.99), 2.665 (1.44), 2.669 (1.97), 2.673 (1.44), 3.245 (0.97), 3.257 (1.07), 3.274 (1.81), 3.281 (1.67), 3.370 (1.51), 3.388 (2.11), 3.405 (2.18), 3.417 (1.57), 3.422 (3.62), 3.435 (3.62), 3.440 (3.45), 3.452 (3.48), 3.457 (1.17), 3.469 (1.10), 3.499 (1.07), 3.535 (1.24), 3.547 (1.04), 3.589 (0.97), 3.716 (1.24), 3.771 (10.64), 3.802 (1.64), 3.816 (1.37), 4.202 (0.57), 4.217 (0.97), 4.226 (1.27), 4.242 (2.74), 4.260 (3.51), 4.274 (2.61), 4.277 (2.54), 4.344 (2.38), 4.356 (4.52), 4.369 (2.18), 4.582 (0.87), 4.623 (0.80), 5.052 (0.54), 5.087 (0.50), 5.345 (0.94), 5.387 (1.44), 5.522 (1.51), 5.564 (0.94), 5.759 (4.02), 6.446 (1.17), 6.466 (1.24), 6.804 (0.40), 6.821 (0.54), 6.837 (1.71), 6.855 (1.87), 6.916 (2.01), 6.934 (2.64), 6.949 (1.14), 6.969 (0.67), 7.028 (1.77), 7.048 (2.71), 7.066 (1.84), 7.075 (1.71), 7.090 (3.25), 7.108 (0.67), 7.168 (0.77), 7.381 (1.94), 7.402 (3.65), 7.421 (2.88), 7.461 (3.92), 7.482 (2.18), 7.504 (0.60), 7.517 (1.77), 7.523 (2.78), 7.531 (3.15), 7.541 (2.91), 7.546 (1.87), 7.559 (0.74), 7.727 (0.50), 7.746 (2.11), 7.765 (1.64), 7.868 (2.14), 7.878 (0.90), 7.885 (1.74), 7.892 (1.71), 8.267 (1.41), 8.273 (1.14), 8.290 (1.10).

### Intermediate 36

### (rac)-Ethyl 4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylate

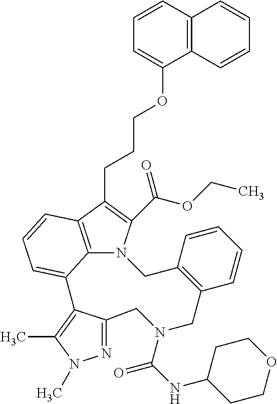 [SMILES: CCOC(=O)c1c(CCCOc2cccc3ccccc23)c2cccc3c2n1Cc1ccccc1CN(C(=O)NC1CCOCC1)Cc1nn(C)c(C)c1-3]

To a solution of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 150 mg, 251 μmol) in dichloromethane (1.6 mL), 4-isocyanatotetrahydro-2H-pyran (CAS 53035-92-0, 38.2 mg, 301 μmol) and N,N-diisopropylethylamine (170 μl, 1.0 mmol) were added. After 17 hours stirring at ambient temperature, all volatiles were removed by evaporation and the residue was subjected to flash chromatography (Biotage SNAP cartridge silica, dichloromethane/ethanol gradient, 0%->10% ethanol) to give the title compound (180 mg).

LC-MS (Method 2): Rₜ=1.63 min, MS (ESIpos): m/z=727 [M+H]⁺

¹H-NMR (400 MHz, DMSO-d6) δ [ppm]: 0.902 (0.76), 1.035 (8.24), 1.053 (16.00), 1.070 (7.74), 1.142 (0.90), 1.159 (0.90), 1.195 (2.08), 1.213 (4.44), 1.230 (2.65), 1.416 (0.44), 1.437 (0.61), 1.445 (0.61), 1.465 (0.50), 1.475 (0.42), 1.708 (0.80), 1.740 (0.69), 1.770 (4.60), 2.066 (0.88), 2.260 (0.40), 2.276 (0.61), 2.287 (0.61), 2.518 (4.25), 2.522 (2.77), 3.305 (0.88), 3.311 (0.80), 3.363 (1.14), 3.404 (1.35), 3.418 (1.20), 3.422 (3.76), 3.435 (3.78), 3.440 (3.32), 3.452 (3.45), 3.457 (1.13), 3.469 (1.11), 3.683 (0.61), 3.719 (0.53), 3.793 (9.19), 3.819 (0.71), 3.829 (0.78), 3.836 (0.74), 3.847 (0.57), 4.195 (0.93), 4.213 (1.39), 4.227 (1.13), 4.238 (0.97), 4.253 (0.59), 4.342 (2.42), 4.355 (4.65), 4.368 (2.25), 5.405 (1.85), 6.523 (0.42), 6.542 (0.46), 6.814 (1.26), 6.816 (1.28), 6.831 (1.43), 6.834 (1.35), 6.898 (1.13), 6.915 (1.24), 6.930 (0.40), 6.950 (0.78), 6.968 (0.46), 7.032 (1.26), 7.050 (1.30), 7.052 (1.43), 7.057 (0.63), 7.070 (1.14), 7.079 (0.99), 7.095 (0.53), 7.197 (1.11), 7.200 (1.13), 7.217 (0.84), 7.219 (0.80), 7.380 (0.92), 7.400 (1.72), 7.419 (1.45), 7.459 (1.74), 7.480 (0.95), 7.497 (0.42), 7.509 (1.05), 7.514 (0.95), 7.519 (1.20), 7.527 (2.42), 7.534 (1.14), 7.538 (1.05), 7.543 (1.13), 7.555 (0.44), 7.724 (1.24), 7.727 (1.32), 7.744 (1.20), 7.747 (1.11), 7.866 (0.99), 7.874 (0.53), 7.884 (0.92), 7.889 (0.84), 8.246 (0.84), 8.252 (0.78), 8.263 (0.42), 8.271 (0.78).

### Intermediate 37

### Ethyl 4,5-dimethyl-17-[3-(naphthalen-1-yloxy) propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylate

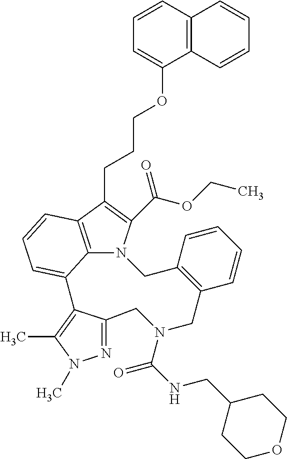 [SMILES: CCOC(=O)c1c(CCCOc2cccc3ccccc23)c2cccc3c2n1Cc1ccccc1CN(C(=O)NCC1CCOCC1)Cc1nn(C)c(C)c1-3]

To a solution of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 150 mg, 251 μmol) in dichloromethane (1.6 mL), 4-(isocyanatomethyl)-tetrahydro-2H-pyran (CAS 934570-48-6, 42.4 mg, 301 μmol) and N,N-diisopropylethylamine (170 μl, 1.0 mmol) were added. After 4 hours stirring at ambient temperature, all volatiles were removed by evaporation and the residue was subjected to flash chromatography (Biotage SNAP cartridge silica, dichloromethane/ethanol gradient, 0%->10% ethanol) to give the title compound (173 mg).

LC-MS (Method 2): Rₜ=1.62 min, MS (ESIpos): m/z=741 [M+H]⁺

¹H-NMR (400 MHz, DMSO-d6) δ [ppm]: 0.884 (0.89), 0.902 (1.88), 0.920 (0.94), 1.009 (0.45), 1.035 (8.27), 1.053 (16.00), 1.070 (8.40), 1.107 (0.42), 1.125 (1.03), 1.137 (1.41), 1.154 (1.65), 1.165 (1.14), 1.194 (4.69), 1.212 (9.21), 1.230 (5.21), 1.571 (0.89), 1.598 (1.34), 1.625 (0.78), 1.662 (0.45), 1.680 (0.54), 1.689 (0.63), 1.697 (0.49), 1.716 (0.40), 1.760 (10.46), 2.066 (2.55), 2.259 (0.80), 2.275 (1.23), 2.289 (1.25), 2.304 (0.85), 2.322 (1.27), 2.327 (1.45), 2.331 (1.03), 2.336 (0.51), 2.419 (0.67), 2.438 (0.78), 2.456 (0.85), 2.518 (4.40), 2.523 (3.11), 2.660 (0.45), 2.665 (0.96), 2.669 (1.32), 2.673 (0.92), 2.865 (0.54), 2.883 (0.92), 2.900 (0.89), 2.914 (0.54), 2.967 (0.49), 2.981 (0.80), 2.998 (0.72), 3.015 (0.51), 3.231 (1.34), 3.236 (0.89), 3.260 (2.46), 3.289 (1.72), 3.307 (1.05), 3.346 (1.72), 3.360 (1.43), 3.381 (0.63), 3.398 (1.09), 3.405 (1.70), 3.417 (1.52), 3.423 (3.69), 3.435 (3.82), 3.440 (3.62), 3.452 (3.62), 3.457 (1.27), 3.470 (1.21), 3.681 (1.12), 3.716 (1.21), 3.776 (15.40), 3.831 (1.65), 3.836 (1.52), 3.854 (1.36), 3.860 (1.45), 4.168 (0.47), 4.177 (0.69), 4.196 (2.10), 4.207 (2.37), 4.213 (3.04), 4.225 (2.44), 4.238 (1.74), 4.252 (1.09), 4.262 (0.67), 4.344 (2.68), 4.357 (5.14), 4.370 (2.50), 4.418 (0.42), 4.449 (0.40), 4.577 (0.58), 4.615 (0.56), 5.413 (3.84), 5.760 (0.80), 6.518 (1.12), 6.537 (1.23), 6.568 (0.87), 6.809 (2.17), 6.811 (2.17), 6.827 (2.46), 6.829 (2.30), 6.898 (2.06), 6.916 (2.28), 6.929 (0.85), 6.949 (1.61), 6.966 (0.92), 7.034 (2.01), 7.054 (3.33), 7.072 (3.42), 7.090 (0.96), 7.181 (1.99), 7.183 (1.99), 7.200 (1.54), 7.379 (1.54), 7.400 (2.91), 7.419 (2.32), 7.459 (3.15), 7.479 (1.74), 7.491 (0.51), 7.495 (0.69), 7.509 (1.77), 7.512 (1.68), 7.518 (1.97), 7.525 (3.78), 7.533 (1.94), 7.538 (1.74), 7.542 (1.85), 7.555 (0.72), 7.559 (0.45), 7.728 (2.17), 7.731 (2.21), 7.749 (2.08), 7.751 (1.97), 7.866 (1.83), 7.873 (0.98), 7.883 (1.70), 7.889 (1.50), 8.245 (1.52), 8.251 (1.47), 8.269 (1.43).

### Intermediate 38

### 2-(Morpholin-4-yl)ethane-1-sulfonyl chloride-hydrochloric Acid Salt

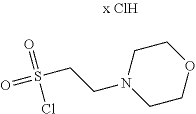 [SMILES: O=S(=O)(Cl)CCN1CCOCC1]

A mixture of 2-(morpholin-4-yl)ethane-1-sulfonic acid (CAS 4432-31-9, 2.20 g, 100% purity, 11.2 mmol) in 100 μL DMF and thionyl chloride (5.3 mL, 73 mmol) was stirred for 3 hours at 75 C. Subsequently, the mixture was concentrated under reduced pressure. The residue was triturated with acetonitrile. The precipitated product was isolated by vacuum filtration to give 1.57 g of the title compound.

### Intermediate 39

### (rac)-Ethyl 4,5-dimethyl-8-{[2-(morpholin-4-yl)ethyl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate

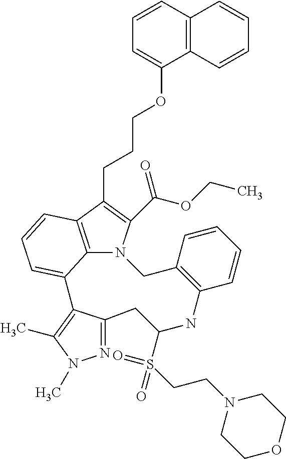 [SMILES: CCOC(=O)c1c(CCCOc2cccc3ccccc23)c2cccc3c2n1Cc1ccccc1NC(S(=O)(=O)CCN1CCOCC1)Cc1nn(C)c(C)c1-3]

2-(Morpholin-4-yl)ethanesulfonyl chloride-hydrochloric acid salt (see intermediate 38, 84.6 mg) and N,N-diisopropylethylamine (110 μl) were added to a solution of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 158 mg) in acetonitrile (3.7 mL). After stirring for 15 hours at ambient temperature, the reaction mixture was evaporated to dryness and the residue was subjected to flash chromatography (Biotage SNAP cartridge silica, dichloromethane/ethanol gradient, 0%->10% ethanol) to give the title compound (149 mg).

LC-MS (Method 2): Rₜ=1.70 min, MS (ESIpos): m/z=777 [M+H]⁺

¹H-NMR (400 MHz, DMSO-d6) δ [ppm]: 0.884 (0.73), 0.902 (1.65), 0.920 (0.82), 1.052 (0.67), 1.070 (0.43), 1.232 (1.46), 1.250 (4.97), 1.267 (9.97), 1.285 (4.75), 1.702 (11.89), 2.066 (2.01), 2.282 (0.85), 2.299 (1.31), 2.310 (1.31), 2.318 (1.55), 2.322 (2.04), 2.327 (2.62), 2.331 (1.86), 2.420 (0.94), 2.439 (1.92), 2.456 (3.81), 2.468 (4.45), 2.518 (6.31), 2.523 (4.51), 2.659 (0.61), 2.665 (1.31), 2.669 (1.83), 2.673 (1.31), 2.678 (0.61), 2.780 (0.67), 2.801 (0.61), 2.824 (0.70), 3.346 (1.01), 3.363 (1.58), 3.381 (1.58), 3.397 (0.79), 3.413 (0.49), 3.429 (1.83), 3.469 (1.80), 3.578 (3.87), 3.589 (6.46), 3.601 (3.99), 3.632 (1.07), 3.820 (16.00), 3.863 (1.52), 4.199 (0.70), 4.207 (0.85), 4.223 (1.46), 4.237 (1.37), 4.250 (2.32), 4.261 (1.49), 4.273 (1.83), 4.279 (2.50), 4.291 (2.59), 4.296 (2.29), 4.308 (1.77), 4.326 (0.55), 4.336 (0.43), 4.613 (1.71), 4.653 (1.58), 5.259 (1.37), 5.300 (1.92), 5.492 (1.31), 5.534 (0.98), 5.760 (5.91), 6.412 (0.79), 6.430 (0.82), 6.821 (2.01), 6.823 (2.04), 6.839 (2.35), 6.898 (2.16), 6.916 (2.38), 6.934 (0.85), 6.952 (1.55), 6.970 (0.88), 7.021 (2.10), 7.041 (2.59), 7.059 (1.71), 7.106 (1.07), 7.126 (1.95), 7.144 (1.01), 7.358 (2.04), 7.376 (1.80), 7.379 (1.80), 7.382 (2.13), 7.403 (3.23), 7.422 (2.68), 7.462 (3.35), 7.483 (1.86), 7.498 (0.46), 7.503 (0.76), 7.515 (2.01), 7.520 (2.04), 7.523 (2.68), 7.531 (4.36), 7.540 (2.80), 7.547 (2.29), 7.559 (0.79), 7.564 (0.46), 7.733 (2.47), 7.735 (2.50), 7.753 (2.35), 7.755 (2.22), 7.869 (1.92), 7.878 (1.01), 7.887 (1.52), 7.892 (1.65), 8.262 (1.65), 8.268 (1.43), 8.277 (0.79), 8.285 (1.52).

### Intermediate 40

### Ethyl 6-chloro-7-{3-ethyl-5-(hydroxymethyl)-1-[2-(morpholin-4-yl)ethyl]-1H-pyrazol-4-yl}-3-[3-(naphthalen-1-yloxy)propyl]-1H-indole-2-carboxylate

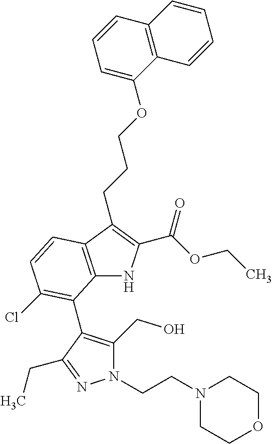 [SMILES: CCOC(=O)c1[nH]c2c(-c3c(CC)nn(CCN4CCOCC4)c3CO)c(Cl)ccc2c1CCCOc1cccc2ccccc12]

The following procedure was performed three times, each reaction with a third of the given amount of material.

{4-Bromo-3-ethyl-1-[2-(morpholin-4-yl)ethyl]-1H-pyrazol-5-yl}methanol (see intermediate 10, 1.00 g, 3.14 mmol) and ethyl 6-chloro-3-[3-(naphthalen-1-yloxy)propyl]-7-(4,4,5,5-tetramethyl-1,3,2-dioxaborolan-2-yl)-1H-indole-2-carboxylate (see intermediate 5, 2.01 g, 3.77 mmol) were dissolved in 1,4 dioxane (39 mL, 460 mmol). The mixture then was purged with argon for three minutes. Afterwards XPhos Pd G3 (326 mg, 98% purity, 377 μmol) and an aqueous solution of potassium phosphate (15 mL, 0.50 M, 7.5 mmol) were added and the mixture was heated to 100° C. for one hour in a microwave reactor. After cooling, the mixture was diluted with ethyl acetate and was washed with a saturated aqueous sodium chloride solution. After drying of the organic phase with sodium sulfate and filtration the filtrate was evaporated to dryness and subjected to flash chromatography (Biotage SNAP cartridge silica, dichloromethane/acetone gradient, 6%->40% acetone) to give the title compound (1.22 g).

LC-MS (Method 2): Rt=1.63 min, MS (ESIpos): m/z=646 [M+H]⁺

¹H-NMR (400 MHz, DMSO-d6) δ [ppm]: 0.920 (6.67), 0.939 (16.00), 0.958 (7.01), 1.066 (1.33), 1.112 (1.56), 1.131 (3.72), 1.138 (0.41), 1.150 (1.56), 1.156 (0.48), 1.239 (6.83), 1.256 (15.78), 1.274 (6.96), 2.084 (0.45), 2.202 (1.09), 2.223 (1.50), 2.231 (1.20), 2.238 (1.21), 2.250 (1.37), 2.268 (1.62), 2.287 (2.23), 2.306 (2.16), 2.321 (1.13), 2.325 (1.00), 2.332 (0.51), 2.340 (2.18), 2.359 (2.24), 2.378 (1.62), 2.397 (1.92), 2.408 (1.29), 2.415 (1.28), 2.426 (1.66), 2.438 (3.04), 2.453 (2.73), 2.460 (1.86), 2.465 (1.60), 2.479 (2.40), 2.518 (2.52), 2.523 (1.51), 2.632 (0.54), 2.648 (1.10), 2.665 (0.89), 2.669 (0.68), 2.673 (0.45), 2.773 (1.49), 2.789 (3.27), 2.807 (1.78), 3.306 (1.46), 3.344 (1.45), 3.530 (0.94), 3.542 (1.28), 3.559 (4.13), 3.571 (7.38), 3.583 (3.99), 4.055 (1.25), 4.088 (1.46), 4.199 (2.08), 4.214 (3.80), 4.223 (1.21), 4.233 (2.35), 4.250 (3.53), 4.252 (4.11), 4.268 (3.81), 4.270 (4.02), 4.280 (0.72), 4.289 (2.39), 4.297 (1.23), 4.305 (0.96), 4.314 (1.72), 4.331 (0.99), 4.349 (0.80), 4.359 (1.40), 4.392 (1.15), 4.450 (0.64), 4.461 (0.64), 5.628 (0.74), 5.759 (3.80), 6.908 (2.16), 6.925 (2.34), 7.167 (5.23), 7.188 (5.20), 7.375 (1.81), 7.395 (3.29), 7.414 (2.82), 7.452 (3.20), 7.473 (1.79), 7.484 (0.61), 7.489 (0.84), 7.501 (2.02), 7.506 (1.81), 7.508 (0.94), 7.513 (2.16), 7.519 (4.36), 7.525 (2.15), 7.532 (1.90), 7.537 (2.16), 7.549 (0.86), 7.553 (0.55), 7.711 (4.17), 7.732 (3.74), 7.860 (1.87), 7.868 (1.06), 7.879 (2.00), 7.884 (1.61), 8.208 (1.68), 8.213 (1.59), 8.226 (0.87), 8.230 (1.48), 8.232 (1.59), 10.637 (2.11).

### Intermediate 41

### (rac)-Ethyl 3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylate

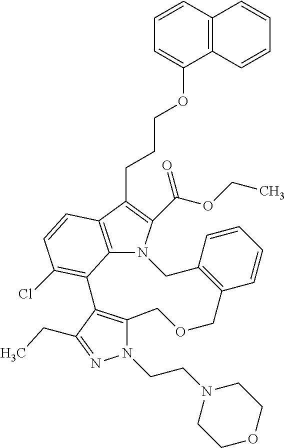 [SMILES: CCOC(=O)c1c(CCCOc2cccc3ccccc23)c2ccc(Cl)c3c2n1Cc1ccccc1COCc1c-3c(CC)nn1CCN1CCOCC1]

To a mixture of ethyl 6-chloro-7-{3-ethyl-5-(hydroxymethyl)-1-[2-(morpholin-4-yl)ethyl]-1H-pyrazol-4-yl}-3-[3-(naphthalen-1-yloxy)propyl]-1H-indole-2-carboxylate (see intermediate 40, 250 mg, 387 μmol), acetonitrile (9.6 mL) and tetrahydrofurane (9.6 mL), cesium carbonate (631 mg, 1.94 mmol) was added and the mixture was stirred for 10 minutes. After addition of 1,2-bis(bromomethyl)benzene (CAS 91-13-4, 118 mg, 95% purity, 426 μmol), the mixture was stirred 15 hours at ambient temperature followed by six hours at 60° C. and two hours at 80° C. After cooling, the mixture was evaporated to dryness and the residue was subjected to flash chromatography (Biotage SNAP cartridge silica, dichloromethane/acetone gradient, 0%->20% acetone) to give the title compound (226 mg).

LC-MS (Method 2): Rₜ=1.77 min, MS (ESIpos): m/z=748 [M+H]⁺

¹H-NMR (400 MHz, DMSO-d6) δ [ppm]: 0.878 (6.76), 0.897 (16.00), 0.916 (7.18), 1.066 (0.59), 1.132 (7.00), 1.150 (15.91), 1.168 (7.31), 1.230 (0.54), 2.083 (6.03), 2.099 (2.34), 2.116 (3.36), 2.127 (3.92), 2.137 (6.09), 2.154 (5.12), 2.172 (2.15), 2.322 (1.21), 2.326 (1.73), 2.331 (1.67), 2.336 (1.43), 2.347 (1.63), 2.375 (1.43), 2.518 (11.43), 2.522 (8.37), 2.551 (1.82), 2.565 (0.87), 2.634 (0.67), 2.652 (1.52), 2.659 (0.82), 2.664 (1.56), 2.669 (2.15), 2.673 (1.54), 2.678 (0.80), 2.684 (1.11), 2.703 (0.50), 3.111 (0.69), 3.128 (0.91), 3.145 (1.19), 3.165 (0.63), 3.205 (0.67), 3.224 (1.26), 3.241 (0.93), 3.258 (0.80), 3.276 (0.41), 3.377 (4.47), 3.388 (8.17), 3.399 (4.66), 3.870 (2.56), 3.890 (2.06), 3.905 (2.78), 3.918 (2.73), 4.047 (0.41), 4.063 (1.08), 4.073 (3.34), 4.086 (1.95), 4.101 (3.08), 4.122 (2.36), 4.138 (4.12), 4.144 (4.08), 4.156 (3.69), 4.162 (3.51), 4.174 (1.34), 4.180 (1.02), 4.189 (0.59), 4.241 (1.17), 4.261 (2.23), 4.276 (1.56), 4.764 (2.49), 4.799 (2.43), 5.167 (0.95), 5.206 (3.30), 5.225 (3.17), 5.263 (1.02), 5.759 (0.72), 6.797 (2.73), 6.815 (2.97), 6.881 (1.56), 6.901 (2.19), 7.124 (0.76), 7.130 (0.89), 7.139 (1.89), 7.146 (3.01), 7.150 (1.84), 7.162 (7.35), 7.169 (3.64), 7.181 (2.34), 7.193 (0.52), 7.200 (0.50), 7.246 (6.09), 7.267 (6.57), 7.353 (2.21), 7.374 (3.88), 7.393 (3.12), 7.445 (3.90), 7.466 (2.58), 7.472 (1.26), 7.486 (2.32), 7.489 (2.15), 7.493 (1.19), 7.505 (4.64), 7.510 (4.83), 7.524 (2.34), 7.528 (2.64), 7.542 (1.30), 7.545 (1.02), 7.775 (5.38), 7.796 (5.01), 7.853 (2.38), 7.872 (2.67), 7.876 (2.17), 8.169 (2.08), 8.172 (2.21), 8.189 (1.78), 8.191 (2.12).

### Intermediate 42

### Ethyl 6-chloro-7-[3-(hydroxymethyl)-1,5-dimethyl-1H-pyrazol-4-yl]-3-[3-(naphthalen-1-yloxy)propyl]-1H-indole-2-carboxylate

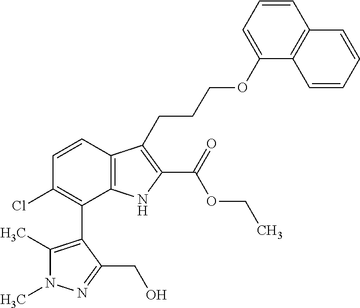 [SMILES: CCOC(=O)c1[nH]c2c(-c3c(CO)nn(C)c3C)c(Cl)ccc2c1CCCOc1cccc2ccccc12]

The reaction was performed in four identical preparations using a quarter of all materials.

Ethyl 6-chloro-3-[3-(naphthalen-1-yloxy)propyl]-7-(4,4,5,5-tetramethyl-1,3,2-dioxaborolan-2-yl)-1H-indole-2-carboxylate (see intermediate 5, 10.0 g, 18.7 mmol) and (4-bromo-1,5-dimethyl-1H-pyrazol-3-yl)methanol (see intermediate 12, 3.84 g, 18.7 mmol) were dissolved in 40 mL of 1,4-dioxane and 20 mL of water and purged with argon for 5 min. Potassium phosphate (9.54 g, 45.0 mmol) and Xphos Pd G3 (1.90 g, 2.25 mmol) were added and the mixture was purged with argon for 5 min and heated for 30 min. to 100° C. in a microwave reactor. The four reaction mixtures was diluted with water and extracted with ethyl acetate. The combined organic phases were washed with brine, dried using a water resistant filter, and concentrated under reduced pressure. The crude product was purified by flash chromatography using silica gel (gradient dichloromethane/ethanol) to obtain the title compound (6.58 g, 59% yield).

LC-MS (Method 1): Rₜ=1.57 min, MS (ESIpos): m/z=532 [M+H]⁺

¹H-NMR (400 MHz, DMSO-d6) δ [ppm]: 1.066 (16.00), 1.138 (1.09), 1.241 (1.06), 1.259 (2.41), 1.277 (1.08), 1.997 (3.38), 2.116 (0.49), 2.205 (0.91), 2.518 (0.47), 3.565 (3.58), 3.725 (0.74), 3.800 (3.23), 3.939 (2.88), 4.214 (0.62), 4.241 (0.56), 4.244 (0.63), 4.259 (0.59), 4.261 (0.56), 7.162 (0.91), 7.183 (0.92), 7.398 (0.52), 7.417 (0.43), 7.453 (0.53), 7.521 (0.74), 7.693 (0.59), 7.715 (0.55), 10.885 (0.44).

### Intermediate 43

### Ethyl 1-[2-(bromomethyl)benzyl]-6-chloro-7-[3-(hydroxymethyl)-1,5-dimethyl-1H-pyrazol-4-yl]-3-[3-(naphthalen-1-yloxy)propyl]-1H-indole-2-carboxylate

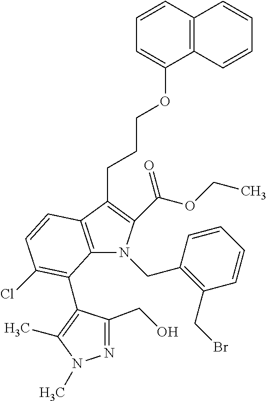 [SMILES: CCOC(=O)c1c(CCCOc2cccc3ccccc23)c2ccc(Cl)c(-c3c(CO)nn(C)c3C)c2n1Cc1ccccc1CBr]

To a solution of ethyl 6-chloro-7-[3-(hydroxymethyl)-1,5-dimethyl-1H-pyrazol-4-yl]-3-[3-(naphthalen-1-yloxy)propyl]-1H-indole-2-carboxylate (see intermediate 42, 2.00 g, 3.76 mmol) in 40 mL of THF and 60 mL of acetonitrile, cesium carbonate (6.12 g, 18.8 mmol) was added and the reaction mixture was stirred for 10 min. at rt. Afterwards, 1,2-bis(bromomethyl)benzene (CAS 91-13-4, 1.19 g, 4.51 mmol) was added and the mixture was stirred for 2 h at rt and was then filtered through celite. The filter cake was washed with acetonitrile and the filtrate was concentrated under reduced pressure to give 2.85 g (75% purity) of the title compound. The crude material was used without further purification in the next step.

LC-MS (Method 1): Rₜ=1.71 min, MS (ESIpos): m/z=714 [M+H]⁺

¹H-NMR (400 MHz, DMSO-d6) δ [ppm]: 0.899 (1.75), 1.066 (16.00), 1.107 (0.75), 1.124 (1.59), 1.137 (0.98), 1.142 (0.89), 1.152 (0.57), 1.169 (1.21), 1.187 (0.54), 1.901 (1.56), 2.074 (0.70), 2.115 (0.47), 2.205 (0.94), 2.518 (1.00), 2.522 (0.65), 3.456 (2.20), 3.725 (0.87), 3.835 (1.60), 3.938 (1.12), 4.145 (0.47), 4.163 (0.72), 4.181 (0.51), 4.284 (0.48), 4.832 (4.82), 7.240 (0.64), 7.262 (0.66), 7.269 (0.43), 7.290 (0.47), 7.338 (0.63), 7.347 (0.63), 7.352 (0.53), 7.361 (0.88), 7.418 (0.41), 7.471 (0.92), 7.481 (0.60), 7.486 (0.73), 7.495 (0.59), 7.520 (0.45), 7.523 (0.47), 7.544 (0.42), 7.855 (0.64), 7.869 (0.42), 7.877 (0.55).

### Intermediate 44

### (rac)-Ethyl 3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylate

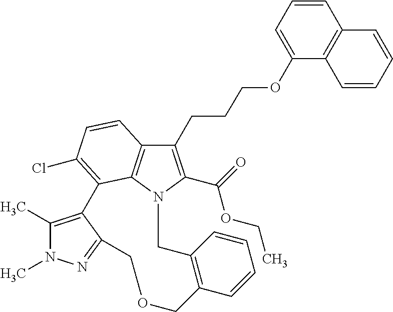 [SMILES: CCOC(=O)c1c(CCCOc2cccc3ccccc23)c2ccc(Cl)c3c2n1Cc1ccccc1COCc1nn(C)c(C)c1-3]

Di-tert-butyl imidodicarbonate (CAS 51779-32-9, 1.73 g, 7.97 mmol) was dissolved in 100 mL of THF. Sodium hydride (261 mg, 55% in mineral oil, 5.98 mmol) was added portionwise and the mixture was stirred for 1 hour at rt. Ethyl 1-[2-(bromomethyl)benzyl]-6-chloro-7-[3-(hydroxymethyl)-1,5-dimethyl-1H-pyrazol-4-yl]-3-[3-(naphthalen-1-yloxy)propyl]-1H-indole-2-carboxylate (see intermediate 43, 2.85 g, 75% purity, 2.99 mmol) was added and the mixture was stirred at rt for 72 hours. The reaction mixture was diluted with saturated, aqueous ammonium chloride solution and extracted with ethyl acetate. The combined organic layers were dried and concentrated under reduced pressure. The crude material was purified by flash chromatography using silica gel (gradient dichloromethane/acetone) to obtain 1.48 g (76% yield) of the title compound.

LC-MS (Method 1): Rₜ=1.70 min, MS (ESIpos): m/z=634 [M+H]⁺

¹H-NMR (400 MHz, DMSO-d6) δ [ppm]: 1.152 (4.97), 1.169 (11.50), 1.187 (5.04), 1.232 (0.69), 1.249 (0.72), 1.363 (12.07), 1.403 (1.31), 1.901 (15.55), 2.080 (0.87), 2.097 (1.34), 2.114 (0.94), 2.518 (3.78), 2.522 (2.42), 3.066 (0.49), 3.083 (0.54), 3.100 (0.84), 3.190 (0.87), 3.207 (0.57), 3.224 (0.54), 3.835 (16.00), 3.874 (1.36), 3.899 (1.81), 4.033 (0.67), 4.047 (2.32), 4.056 (2.92), 4.072 (1.95), 4.088 (2.30), 4.100 (1.11), 4.108 (0.49), 4.115 (0.59), 4.124 (0.62), 4.145 (1.29), 4.162 (4.06), 4.181 (3.98), 4.198 (1.16), 4.408 (2.25), 4.440 (1.98), 5.140 (1.26), 5.177 (1.85), 5.297 (1.76), 5.333 (1.31), 5.758 (2.13), 6.783 (1.68), 6.801 (1.83), 7.065 (1.09), 7.069 (1.01), 7.083 (2.15), 7.086 (2.28), 7.099 (1.38), 7.103 (1.73), 7.147 (0.54), 7.152 (0.72), 7.166 (1.58), 7.170 (1.46), 7.183 (2.55), 7.188 (2.32), 7.200 (1.38), 7.205 (1.19), 7.219 (0.42), 7.269 (4.03), 7.290 (4.60), 7.356 (1.41), 7.376 (2.50), 7.395 (2.03), 7.443 (2.45), 7.464 (1.53), 7.471 (0.74), 7.485 (1.46), 7.489 (1.36), 7.492 (0.69), 7.504 (2.55), 7.508 (2.72), 7.520 (0.74), 7.523 (1.46), 7.527 (1.66), 7.540 (0.77), 7.544 (0.57), 7.752 (3.64), 7.773 (3.36), 7.850 (1.46), 7.855 (0.99), 7.869 (1.68), 7.874 (1.26), 8.151 (1.34), 8.155 (1.36), 8.171 (1.01), 8.173 (1.26), 8.175 (1.24).

### Intermediate 45

### Ethyl 3-chloro-4,5-dimethyl-17-[3-(naphthalen-1-yloxy)propyl]-5,7,9,14-tetrahydropyrazolo[4′,3′:9,10]pyrido[3′,2′:3,4][1,6]oxazacycloundecino[8,7,6-hi]indole-16-carboxylate

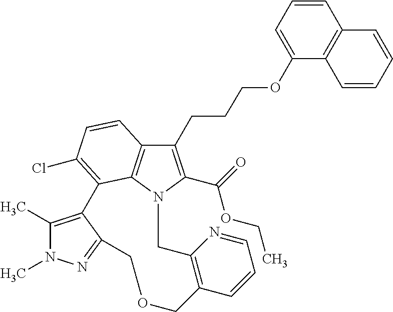 [SMILES: CCOC(=O)c1c(CCCOc2cccc3ccccc23)c2ccc(Cl)c3c2n1Cc1ncccc1COCc1nn(C)c(C)c1-3]

Ethyl 6-chloro-7-[3-(hydroxymethyl)-1,5-dimethyl-1H-pyrazol-4-yl]-3-[3-(naphthalen-1-yloxy)propyl]-1H-indole-2-carboxylate (see intermediate 42, 500 mg, 940 μmol) was dissolved in 10 mL of acetonitrile. Cesium carbonate (1.84 g, 5.64 mmol) was added and the mixture was stirred for 10 min. at rt. 2,3-Bis(chloromethyl)pyridine hydrochloride (CAS 27221-49-4, 300 mg, 1.41 mmol) and sodium iodide (282 mg, 1.88 mmol) were added and the mixture was stirred for 24 hours at 75° C. in a sealed tube. 2,3-Bis(chloromethyl)pyridine hydrochloride (100 mg, 0.47 mmol) and sodium iodide (71 mg, 0.47 mmol) were added, and the mixture was stirred for 24 hours at 75° C. in a sealed tube, was diluted with water and extracted with ethyl acetate. The combined organic layers were washed with brine, dried using a water resistant filter, and concentrated under reduced pressure. The crude product was purified by flash chromatography using silica gel (gradient dichloromethane/ethanol) to obtain the title compound (86.6 mg, 10% yield).

LC-MS (Method 1): Rₜ=1.68 min, MS (ESIpos): m/z=635 [M+H]⁺

### Intermediate 46

### Ethyl 3-chloro-4,5-dimethyl-17-[3-(1-naphthyloxy)propyl]-5,7,9,14-tetrahydropyrazolo[4′,3′:9,10]pyrido[2′,3′:3,4][1,6]oxazacycloundecino[8,7,6-hi]indole-16-carboxylate

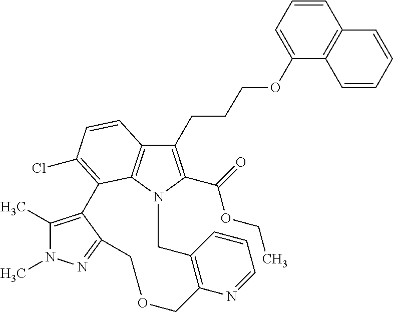 [SMILES: CCOC(=O)c1c(CCCOc2cccc3ccccc23)c2ccc(Cl)c3c2n1Cc1cccnc1COCc1nn(C)c(C)c1-3]

59.9 mg (6% yield) of the title compound were isolated as side product in the preparation of intermediate 45.

LC-MS (Method 1): Rₜ=1.60 min; MS (ESIpos): m/z=635 [M+H]⁺

### Intermediate 47

### 2,3-bis(chloromethyl)pyrazine

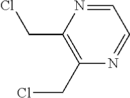

2,3-Dimethylpyrazine (CAS 5910-89-4, 4.8 mL, 45.3 mmol) was dissolved in 70 mL of tetrachloromethane. 1-Chloropyrrolidine-2,5-dione (CAS 128-09-6, 13.3 g, 99.7 mmol) and benzoyl peroxide (549 mg, 2.27 mmol) were added and the mixture was stirred for 25 hours under reflux. After cooling to rt the precipitate was filtered off. The filtrate was concentrated under reduced pressure and the crude material was purified by flash chromatography using silica gel (gradient hexane/ethyl acetate) to give the title compound (4.43 g, 44% yield).

LC-MS (Method 1): Rₜ=0.85 min; MS (ESIpos): m/z=177 [M+H]⁺

¹H NMR (400 MHz, DMSO-d₆) δ [ppm]: 4.98 (s, 4H) 8.68 (s, 2H)

### Intermediate 48

### (rac)-Ethyl 3-chloro-4,5-dimethyl-17-{3-[(naphthalen-1-yl)oxy]propyl}-5,7,9,14-tetrahydropyrazino[2′,3′:3,4]pyrazolo[4′,3′:9,10][1,6]oxazacycloundecino[8,7,6-hi]indole-16-carboxylate

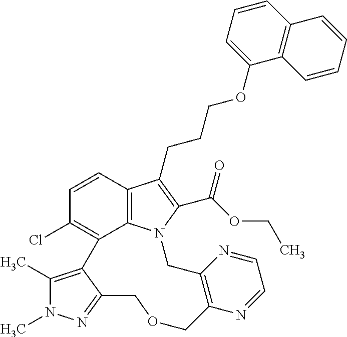 [SMILES: CCOC(=O)c1c(CCCOc2cccc3ccccc23)c2ccc(Cl)c3c2n1Cc1nccnc1COCc1nn(C)c(C)c1-3]

Ethyl 6-chloro-7-[3-(hydroxymethyl)-1,5-dimethyl-1H-pyrazol-4-yl]-3-{3-[(naphthalen-1-yl)oxy]propyl}-1H-indole-2-carboxylate (see intermediate 42, 887 mg, 1.67 mmol) was dissolved in 10 mL of acetonitrile. Cesium carbonate (2.72 g, 8.34 mmol) was added and the mixture was stirred for 10 min. at rt. 2,3-Bis(chloromethyl)pyrazine (see intermediate 47, 354 mg, 80% purity, 1.60 mmol) and sodium iodide (500 mg, 3.33 mmol) were added, and the reaction mixture was heated for 17 hours at 70° C. After cooling to rt, the reaction mixture was diluted with water and extracted with ethyl acetate. The combined organic layers were washed with brine, dried using a water resistant filter and concentrated under reduced pressure. The crude product was purified by flash chromatography using silica gel (gradient dichloromethane/ethanol) to obtain the title compound (277 mg, 50% purity, 13% yield).

LC-MS (Method 1): Rₜ=1.64 min, MS (ESIpos): m/z=636 [M+H]⁺

¹H-NMR (400 MHz, DMSO-d6) δ [ppm]: 0.977 (0.82), 1.066 (16.00), 1.245 (0.56), 1.263 (1.05), 1.281 (0.49), 1.858 (1.39), 2.022 (1.16), 2.204 (8.14), 2.518 (1.06), 2.523 (0.73), 3.725 (7.91), 3.781 (0.48), 3.786 (1.24), 3.871 (1.38), 3.942 (2.34), 4.285 (2.49), 4.299 (2.68), 4.936 (0.66), 4.950 (1.32), 4.964 (0.58), 7.376 (0.47), 7.397 (0.62), 7.452 (0.46), 7.520 (0.44), 8.360 (0.46).

### Intermediate 49

### Ethyl 6-chloro-3-{3-[(6-fluoronaphthalen-1-yl)oxy]propyl}-7-[3-(hydroxymethyl)-1,5-dimethyl-1H-pyrazol-4-yl]-1H-indole-2-carboxylate

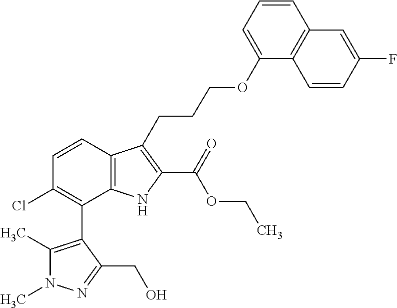 [SMILES: CCOC(=O)c1[nH]c2c(-c3c(CO)nn(C)c3C)c(Cl)ccc2c1CCCOc1cccc2cc(F)ccc12]

Ethyl 6-chloro-3-{3-[(6-fluoronaphthalen-1-yl)oxy]propyl}-7-(4,4,5,5-tetramethyl-1,3,2-dioxa-borolan-2-yl)-1H-indole-2-carboxylate (see intermediate 6, 2.00 g, 3.62 mmol), (4-bromo-1,5-dimethyl-1H-pyrazol-3-yl)methanol (see intermediate 12, 929 mg, 4.53 mmol) and potassium phosphate (2.31 g, 10.9 mmol) were dissolved in 15 mL of 1,4-dioxane and 5 mL of water and purged with argon for 5 min. XPhos Pd G3 (1.07 g, 1.27 mmol) was added, and the mixture was heated for 1 hour at 100° C. in a microwave reactor. The reaction mixture was diluted with ethyl acetate, filtered, diluted with water and extracted with ethyl acetate. The combined organic layers were dried over sodium sulfate, filtered and concentrated under reduced pressure. The crude product was purified by flash chromatography using silica gel (gradient dichloromethane/acetone) to obtain the title compound (943 mg, 46% yield).

LC-MS (Method 1): Rₜ=1.57 min, MS (ESIpos): m/z=550 [M+H]⁺

¹H-NMR (400 MHz, DMSO-d6) δ [ppm]: 1.230 (3.92), 1.248 (8.81), 1.265 (4.02), 1.994 (12.76), 2.084 (16.00), 2.194 (0.79), 2.205 (1.37), 2.210 (1.07), 2.230 (0.82), 2.518 (2.62), 2.523 (1.71), 3.298 (1.04), 3.317 (1.92), 3.725 (0.73), 3.789 (0.58), 3.800 (12.81), 4.039 (0.67), 4.053 (0.68), 4.069 (0.82), 4.082 (0.78), 4.198 (1.14), 4.204 (0.79), 4.213 (2.90), 4.231 (2.61), 4.234 (2.52), 4.248 (2.15), 4.251 (2.07), 4.266 (0.72), 4.269 (0.63), 4.284 (0.95), 4.293 (0.89), 4.299 (0.40), 4.313 (0.73), 4.322 (0.67), 5.173 (0.69), 5.184 (1.15), 5.197 (0.65), 5.759 (0.99), 6.890 (0.99), 6.899 (1.10), 6.912 (1.05), 7.167 (3.24), 7.189 (3.35), 7.369 (0.72), 7.376 (0.83), 7.391 (1.05), 7.398 (1.12), 7.414 (0.69), 7.421 (0.88), 7.440 (2.25), 7.444 (2.33), 7.453 (4.45), 7.648 (1.22), 7.655 (1.24), 7.674 (1.22), 7.681 (1.24), 7.691 (2.33), 7.712 (2.11), 8.235 (1.05), 8.249 (1.10), 8.258 (1.08), 8.273 (1.00), 10.888 (1.88).

### Intermediate 50

### (rac)-Ethyl 3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylate

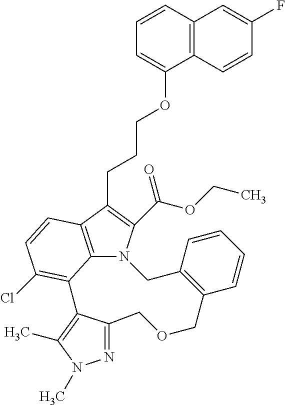 [SMILES: CCOC(=O)c1c(CCCOc2cccc3cc(F)ccc23)c2ccc(Cl)c3c2n1Cc1ccccc1COCc1nn(C)c(C)c1-3]

To a mixture of ethyl 6-chloro-3-{3-[(6-fluoronaphthalen-1-yl)oxy]propyl}-7-[3-(hydroxymethyl)-1,5-dimethyl-1H-pyrazol-4-yl]-1H-indole-2-carboxylate (see intermediate 49, 200 mg, 364 μmol) in 10 mL of acetonitrile and 5 mL of THF was added cesium carbonate (592 mg, 1.82 mmol) and the mixture was stirred for 10 minutes. 1,2-Bis(bromomethyl)benzene (CAS 91-13-4, 106 mg, 400 μmol) was added, and the reaction mixture was stirred for 5 hours at 80° C. The mixture was filtered and concentrated under reduced pressure. The crude product was purified by flash chromatography using silica gel (gradient dichloromethane/acetone) to give the title compound (178 mg, 74% yield).

LC-MS (Method 1): Rₜ=1.72 min, MS (ESIpos): m/z=652 [M+H]⁺

¹H-NMR (400 MHz, DMSO-d6) δ [ppm]: 1.149 (4.59), 1.167 (10.08), 1.184 (4.69), 1.232 (0.42), 1.899 (15.56), 2.057 (0.40), 2.076 (1.10), 2.084 (3.31), 2.093 (1.66), 2.110 (1.12), 2.518 (5.67), 2.523 (3.64), 3.061 (0.58), 3.077 (0.63), 3.095 (0.96), 3.114 (0.42), 3.161 (0.44), 3.180 (0.98), 3.197 (0.65), 3.214 (0.63), 3.835 (16.00), 3.873 (1.54), 3.898 (2.03), 4.030 (0.75), 4.051 (2.94), 4.058 (2.85), 4.076 (1.96), 4.090 (2.50), 4.098 (1.40), 4.113 (0.75), 4.122 (0.75), 4.141 (1.49), 4.159 (4.24), 4.177 (4.17), 4.194 (1.31), 4.410 (2.38), 4.442 (2.10), 5.140 (1.40), 5.177 (2.05), 5.294 (1.96), 5.332 (1.45), 5.759 (0.42), 6.759 (1.33), 6.765 (1.35), 6.775 (1.28), 6.781 (1.42), 7.063 (1.24), 7.067 (1.14), 7.082 (2.47), 7.086 (2.59), 7.101 (1.56), 7.105 (1.91), 7.147 (0.61), 7.151 (0.75), 7.165 (1.75), 7.169 (1.59), 7.182 (2.47), 7.186 (2.29), 7.199 (1.45), 7.203 (1.24), 7.217 (0.44), 7.272 (3.92), 7.293 (3.94), 7.354 (0.84), 7.360 (0.96), 7.376 (1.38), 7.382 (1.52), 7.399 (1.21), 7.405 (1.07), 7.422 (2.22), 7.438 (5.20), 7.454 (0.56), 7.640 (1.52), 7.646 (1.59), 7.666 (1.56), 7.672 (1.56), 7.749 (3.55), 7.770 (3.22), 8.182 (1.33), 8.198 (1.38), 8.206 (1.35), 8.220 (1.33).

### Intermediate 51

### (rac)-Ethyl 3-chloro-17-{3-[(6-fluoronaphthalen-1-yl)oxy]propyl}-4,5-dimethyl-5,7,9,14-tetrahydropyrazino[2′,3′:3,4]pyrazolo[4′,3′:9,10][1,6]oxazacycloundecino[8,7,6-hi]indole-16-carboxylate

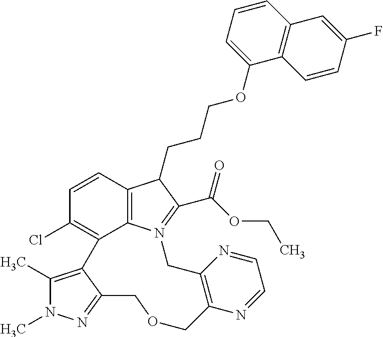

Ethyl 6-chloro-3-{3-[(6-fluoronaphthalen-1-yl)oxy]propyl}-7-[3-(hydroxymethyl)-1,5-dimethyl-1H-pyrazol-4-yl]-1H-indole-2-carboxylate (see intermediate 49, 50.0 mg, 90.9 μmol) was dissolved in 10 mL of acetonitrile. Cesium carbonate (148 mg, 455 μmol) was added and the mixture was stirred 10 min. at rt. 2,3-Bis(chloromethyl)pyrazine (see intermediate 47, 19.3 mg, 80% purity, 87.2 μmol) and sodium iodide (27.3 mg, 182 μmol) were added, and the reaction mixture was heated for 17 hours at 70° C. The reaction mixture was diluted with water and was extracted with ethyl acetate. The combined organic phases were filtered, dried and concentrated under reduced pressure to give 89.2 mg (50% purity, 90% yield) of the title compound which was used in the next step without further purification.

LC-MS (Method 1): Rₜ=1.65 min, MS (ESIpos): m/z=654 [M+H]⁺

¹H-NMR (400 MHz, DMSO-d6) δ [ppm]: 0.879 (5.88), 0.896 (6.12), 1.008 (0.94), 1.026 (1.18), 1.064 (11.06), 1.102 (1.88), 1.147 (6.59), 1.153 (4.47), 1.164 (7.53), 1.171 (5.41), 1.188 (3.53), 1.230 (12.47), 1.237 (5.41), 1.248 (11.53), 1.254 (7.53), 1.272 (3.29), 1.291 (0.94), 1.599 (4.24), 1.784 (0.71), 1.855 (8.47), 1.964 (0.94), 1.986 (6.35), 2.147 (1.18), 2.297 (0.94), 2.332 (3.06), 2.359 (1.18), 2.376 (0.71), 2.518 (16.00), 2.523 (10.59), 2.779 (0.94), 2.852 (0.47), 2.868 (0.71), 2.885 (0.47), 3.219 (0.94), 3.293 (2.35), 3.300 (2.35), 3.388 (4.00), 3.397 (1.65), 3.408 (0.71), 3.428 (0.71), 3.802 (0.47), 3.850 (1.41), 3.870 (8.47), 3.957 (0.94), 3.998 (0.47), 4.015 (1.41), 4.033 (1.41), 4.054 (1.18), 4.091 (0.94), 4.181 (1.65), 4.206 (1.65), 4.234 (0.94), 4.252 (0.94), 4.261 (1.41), 4.279 (1.41), 4.290 (1.18), 4.307 (0.94), 4.373 (1.18), 4.404 (1.41), 4.504 (0.94), 4.528 (0.94), 4.577 (1.18), 4.609 (0.94), 4.900 (0.71), 5.126 (0.71), 5.163 (1.18), 5.368 (0.94), 5.406 (0.94), 6.800 (0.71), 6.809 (0.94), 6.822 (0.71), 6.925 (5.88), 7.083 (0.71), 7.209 (2.12), 7.230 (2.35), 7.370 (0.94), 7.393 (1.41), 7.441 (3.06), 7.450 (4.47), 7.483 (0.94), 7.501 (0.94), 7.647 (3.06), 7.668 (2.82), 7.720 (0.71), 8.206 (0.94), 8.221 (0.94), 8.229 (0.94), 8.245 (0.94), 8.331 (1.65), 8.337 (2.35), 8.352 (2.82), 8.358 (1.65), 8.518 (0.71), 9.769 (0.47).

### Intermediate 52

### Ethyl 6-chloro-7-{3-ethyl-5-(hydroxymethyl)-1-[2-(morpholin-4-yl)ethyl]-1H-pyrazol-4-yl}-3-{3-[(6-fluoronaphthalen-1-yl)oxy]propyl}-1H-indole-2-carboxylate

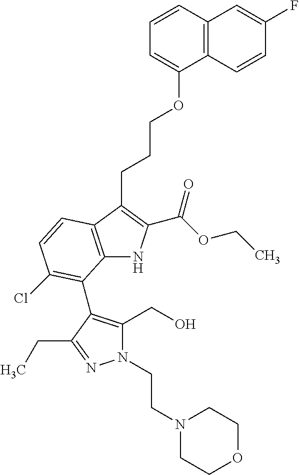 [SMILES: CCOC(=O)c1[nH]c2c(-c3c(CC)nn(CCN4CCOCC4)c3CO)c(Cl)ccc2c1CCCOc1cccc2cc(F)ccc12]

Ethyl 6-chloro-3-{3-[(6-fluoronaphthalen-1-yl)oxy]propyl}-7-(4,4,5,5-tetramethyl-1,3,2-dioxaborolan-2-yl)-1H-indole-2-carboxylate (see intermediate 6, 10.0 g, 18.1 mmol) and {4-bromo-3-ethyl-1-[2-(morpholin-4-yl)ethyl]-1H-pyrazol-5-yl}methanol (see intermediate 10, 6.06 g, 19.0 mmol) were dissolved in 220 mL of THF and an aqueous potassium phosphate solution (72 mL, 0.50 M, 36 mmol) was added. This mixture was purged with argon for 10 min. XPhos Pd G3 (713 mg, 906 μmol) was added and the mixture was heated for 2 hours at 50° C. After concentration under reduced pressure, the residue was extracted with ethyl acetate. The combined organic layers were filtered and concentrated under reduced pressure. The crude product was purified by flash chromatography using silica gel (gradient dichloromethane/ethanol) to obtain the title compound (4.96 g).

LC-MS (Method 2): Rₜ=1.67 min, MS (ESIpos): m/z=663 [M+H]⁺

¹H-NMR (400 MHz, DMSO-d6) δ [ppm]: 0.813 (0.44), 0.830 (0.64), 0.839 (0.42), 0.848 (0.55), 0.856 (0.42), 0.892 (0.65), 0.904 (0.50), 0.909 (0.69), 0.920 (7.03), 0.939 (15.63), 0.951 (1.12), 0.958 (7.08), 1.066 (0.94), 1.131 (0.73), 1.137 (1.24), 1.154 (3.47), 1.172 (5.86), 1.189 (3.03), 1.225 (7.66), 1.236 (2.69), 1.243 (16.00), 1.253 (1.93), 1.261 (7.42), 1.286 (0.58), 1.294 (0.42), 1.305 (0.55), 1.311 (0.62), 1.322 (0.65), 1.339 (0.60), 1.394 (2.41), 1.987 (10.16), 2.200 (1.40), 2.216 (1.88), 2.227 (1.43), 2.235 (1.49), 2.246 (1.54), 2.265 (1.70), 2.284 (2.46), 2.302 (2.28), 2.320 (1.54), 2.327 (1.17), 2.331 (0.92), 2.338 (2.42), 2.357 (2.34), 2.375 (1.61), 2.394 (1.43), 2.413 (0.99), 2.427 (1.96), 2.438 (3.81), 2.452 (3.56), 2.465 (2.39), 2.518 (4.28), 2.523 (2.97), 2.665 (0.90), 2.669 (1.15), 2.673 (0.83), 2.772 (1.88), 2.789 (3.95), 2.806 (2.16), 3.299 (1.93), 3.319 (3.56), 3.559 (4.87), 3.571 (8.58), 3.582 (4.69), 3.999 (0.76), 4.017 (2.25), 4.035 (2.32), 4.053 (1.66), 4.082 (1.27), 4.089 (1.19), 4.198 (2.14), 4.213 (4.30), 4.223 (2.67), 4.228 (2.51), 4.238 (4.34), 4.241 (4.55), 4.256 (4.55), 4.258 (4.11), 4.274 (2.46), 4.290 (2.11), 4.297 (1.49), 4.306 (1.31), 4.314 (2.11), 4.332 (1.27), 4.349 (1.31), 4.363 (1.24), 4.386 (0.96), 4.394 (0.87), 5.632 (1.50), 6.884 (1.88), 6.893 (1.91), 6.898 (1.58), 6.906 (1.93), 6.994 (0.53), 7.173 (5.52), 7.195 (5.58), 7.364 (1.45), 7.371 (1.61), 7.386 (2.09), 7.393 (2.28), 7.408 (1.68), 7.415 (2.07), 7.426 (0.69), 7.438 (3.96), 7.443 (4.14), 7.451 (8.11), 7.463 (0.50), 7.648 (2.34), 7.655 (2.39), 7.674 (2.21), 7.681 (2.12), 7.709 (3.95), 7.731 (3.59), 8.220 (1.96), 8.235 (2.09), 8.243 (2.05), 8.258 (1.91), 10.640 (3.50).

### Intermediate 53

### Ethyl 1-[2-(bromomethyl)benzyl]-6-chloro-7-{3-ethyl-5-(hydroxymethyl)-1-[2-(morpholin-4-yl)ethyl]-1H-pyrazol-4-yl}-3-{3-[(6-fluoronaphthalen-1-yl)oxy]propyl}-1H-indole-2-carboxylate

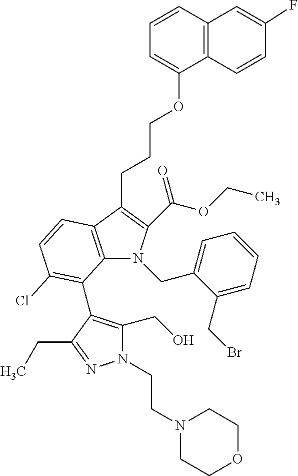 [SMILES: CCOC(=O)c1c(CCCOc2cccc3cc(F)ccc23)c2ccc(Cl)c(-c3c(CC)nn(CCN4CCOCC4)c3CO)c2n1Cc1ccccc1CBr]

Ethyl 6-chloro-7-{3-ethyl-5-(hydroxymethyl)-1-[2-(morpholin-4-yl)ethyl]-1H-pyrazol-4-yl}-3-{3-[(6-fluoronaphthalen-1-yl)oxy]propyl}-1H-indole-2-carboxylate (see intermediate 52, 3.00 g, 4.52 mmol) was dissolved in 50 mL of THF and 75 mL of acetonitrile, cesium carbonate (7.37 g, 22.6 mmol) was added and the reaction mixture was stirred for 10 min. at rt. 1,2-Bis(bromomethyl)benzene (CAS 91-13-4, 1.51 g, 95% purity, 5.43 mmol) was added and the mixture was stirred for 75 min. at 40° C. The reaction mixture was filtered and concentrated under reduced pressure to give the title compound (4.22 g). The crude material was used without further purification in the next step.

LC-MS (Method 2): Rₜ=1.76 min, MS (ESIpos): m/z=845 [M+H]⁺

¹H-NMR (400 MHz, DMSO-d6) δ [ppm]: 0.734 (1.56), 0.752 (3.27), 0.771 (1.70), 0.875 (1.03), 0.894 (2.35), 0.913 (1.08), 0.922 (0.50), 0.941 (0.80), 0.960 (0.42), 1.050 (3.61), 1.067 (8.14), 1.085 (3.77), 1.129 (1.15), 1.137 (0.71), 1.147 (2.57), 1.156 (0.62), 1.164 (1.56), 1.226 (1.17), 1.231 (1.26), 1.244 (1.52), 1.249 (0.92), 1.261 (0.74), 1.304 (0.60), 1.323 (0.81), 1.340 (0.46), 1.352 (2.34), 1.756 (1.08), 1.773 (0.48), 2.074 (3.66), 2.115 (0.51), 2.130 (0.94), 2.148 (0.81), 2.180 (0.42), 2.258 (0.74), 2.274 (1.04), 2.293 (0.83), 2.322 (0.99), 2.326 (1.26), 2.331 (0.97), 2.336 (0.64), 2.358 (0.69), 2.394 (0.88), 2.430 (0.80), 2.518 (4.88), 2.522 (3.27), 2.664 (1.27), 2.668 (1.54), 2.673 (1.20), 3.359 (1.06), 3.380 (1.79), 3.396 (1.36), 3.545 (2.42), 3.582 (0.92), 3.598 (1.13), 3.615 (0.50), 3.871 (0.71), 3.905 (0.71), 4.072 (1.17), 4.090 (2.65), 4.105 (2.76), 4.107 (2.65), 4.123 (1.49), 4.135 (1.06), 4.141 (0.97), 4.152 (0.90), 4.159 (0.94), 4.223 (0.76), 4.248 (1.98), 4.263 (2.67), 4.278 (1.65), 4.311 (0.74), 4.832 (16.00), 4.837 (2.12), 5.128 (1.17), 5.224 (0.41), 5.447 (1.43), 5.534 (0.48), 5.581 (0.67), 5.623 (0.96), 5.668 (0.42), 5.756 (1.06), 5.776 (1.08), 6.898 (0.48), 6.910 (0.97), 6.919 (1.19), 6.931 (1.03), 6.984 (0.53), 7.000 (1.12), 7.020 (0.67), 7.112 (0.65), 7.129 (1.22), 7.148 (0.76), 7.162 (0.96), 7.174 (0.51), 7.195 (0.42), 7.250 (0.87), 7.273 (2.64), 7.295 (2.48), 7.334 (1.38), 7.338 (2.57), 7.347 (2.46), 7.352 (2.99), 7.361 (3.22), 7.371 (0.94), 7.376 (1.20), 7.382 (1.08), 7.399 (1.50), 7.405 (1.75), 7.420 (1.31), 7.427 (1.19), 7.435 (1.50), 7.453 (3.12), 7.463 (4.74), 7.472 (2.69), 7.481 (2.02), 7.486 (1.89), 7.495 (1.96), 7.504 (0.51), 7.654 (1.49), 7.660 (1.43), 7.668 (0.55), 7.674 (0.85), 7.679 (1.40), 7.686 (1.26), 7.772 (0.65), 7.794 (0.58), 7.883 (2.02), 7.905 (1.89), 8.221 (0.51), 8.236 (1.40), 8.252 (1.12), 8.260 (1.22), 8.274 (0.99), 10.316 (0.60).

### Intermediate 54

### (rac)-Ethyl 3-chloro-4-ethyl-17-{3-[(6-fluoro-1-naphthyl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylate

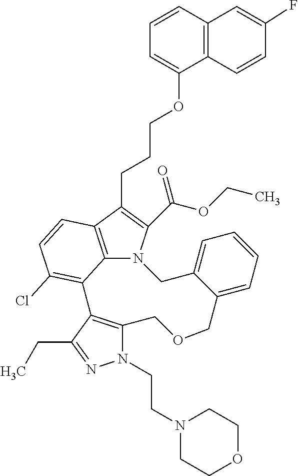 [SMILES: CCOC(=O)c1c(CCCOc2cccc3cc(F)ccc23)c2ccc(Cl)c3c2n1Cc1ccccc1COCc1c-3c(CC)nn1CCN1CCOCC1]

Di-tert-butyl imidodicarbonate (CAS 51779-32-9, 2.05 g, 95% purity, 8.98 mmol) was dissolved in 40 mL of DMF. Sodium hydride (269 mg, 60% in mineral oil, 6.73 mmol) was added portionwise and the reaction mixture was stirred for 1 hour at rt. Ethyl 1-[2-(bromomethyl)benzyl]-6-chloro-7-{3-ethyl-5-(hydroxymethyl)-1-[2-(morpholin-4-yl)ethyl]-1H-pyrazol-4-yl}-3-{3-[(6-fluoronaphthalen-1-yl)oxy]propyl}-1H-indole-2-carboxylate (see intermediate 53, 4.22 g, 90% purity, 4.49 mmol), dissolved in 40 mL of DMF, was added and the mixture was stirred at rt for 16 hours and subsequently at 50° C. for 1 hour. The reaction mixture was poured into ice water and was extracted with ethyl acetate. The combined organic layers were washed with brine, dried using a water resistant filter, and concentrated under reduced pressure. The crude material was purified by flash chromatography twice using silica gel (gradient dichloromethane/acetone and gradient hexane/ethanol) to obtain 340 mg (10% yield) of the title compound.

LC-MS (Method 2): Rₜ=1.79 min, MS (ESIpos): m/z=765 [M+H]⁺

¹H-NMR (400 MHz, DMSO-d6) δ [ppm]: 0.875 (6.91), 0.894 (15.67), 0.913 (7.37), 0.929 (0.63), 1.067 (0.45), 1.128 (7.29), 1.146 (16.00), 1.153 (2.20), 1.163 (7.62), 1.171 (2.91), 1.189 (1.32), 1.229 (0.51), 1.246 (0.66), 1.259 (2.89), 1.986 (4.65), 2.086 (1.28), 2.098 (2.60), 2.111 (4.07), 2.128 (8.03), 2.138 (3.79), 2.147 (6.31), 2.166 (2.64), 2.327 (1.31), 2.331 (1.44), 2.346 (1.91), 2.375 (1.62), 2.518 (4.81), 2.522 (3.24), 2.536 (1.47), 2.551 (1.69), 2.565 (0.93), 2.634 (0.82), 2.651 (1.76), 2.669 (1.90), 2.683 (1.20), 2.702 (0.58), 3.088 (0.43), 3.105 (0.81), 3.122 (1.06), 3.140 (1.43), 3.159 (0.67), 3.195 (0.76), 3.214 (1.50), 3.231 (1.04), 3.248 (0.89), 3.266 (0.44), 3.377 (5.29), 3.388 (9.46), 3.399 (5.26), 3.573 (0.45), 3.870 (2.86), 3.887 (2.30), 3.905 (3.15), 3.915 (3.11), 4.016 (1.09), 4.034 (1.15), 4.045 (0.51), 4.052 (0.67), 4.060 (1.26), 4.073 (3.84), 4.084 (2.34), 4.101 (3.60), 4.122 (2.98), 4.134 (4.32), 4.140 (4.26), 4.151 (3.77), 4.158 (3.57), 4.168 (1.53), 4.176 (1.04), 4.185 (0.60), 4.207 (0.40), 4.226 (0.52), 4.241 (1.61), 4.260 (2.98), 4.766 (2.78), 4.802 (2.69), 5.166 (1.07), 5.204 (3.77), 5.225 (3.57), 5.263 (1.13), 6.771 (2.24), 6.775 (2.30), 6.788 (2.28), 6.792 (2.38), 6.883 (1.99), 6.901 (2.47), 7.121 (0.84), 7.127 (1.07), 7.138 (1.92), 7.144 (2.79), 7.149 (2.06), 7.161 (8.32), 7.179 (2.31), 7.191 (0.44), 7.198 (0.49), 7.249 (5.98), 7.271 (6.12), 7.347 (1.35), 7.353 (1.61), 7.369 (2.24), 7.376 (2.45), 7.391 (1.57), 7.398 (2.49), 7.417 (3.46), 7.434 (7.79), 7.447 (0.77), 7.454 (1.29), 7.641 (2.47), 7.647 (2.58), 7.667 (2.52), 7.673 (2.56), 7.770 (5.47), 7.792 (5.00), 8.198 (2.14), 8.212 (2.24), 8.220 (2.19), 8.236 (2.12)

### Intermediate 55

### Ethyl 6-chloro-7-[5-ethyl-3-(hydroxymethyl)-1-methyl-1H-pyrazol-4-yl]-3-{3-[(6-fluoronaphthalen-1-yl)oxy]propyl}-1H-indole-2-carboxylate

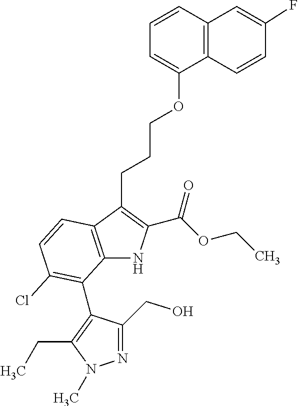 [SMILES: CCOC(=O)c1[nH]c2c(-c3c(CO)nn(C)c3CC)c(Cl)ccc2c1CCCOc1cccc2cc(F)ccc12]

The reaction was performed in four identical preparations using a quarter of all materials.

Ethyl 6-chloro-3-{3-[(6-fluoronaphthalen-1-yl)oxy]propyl}-7-(4,4,5,5-tetramethyl-1,3,2-dioxaborolan-2-yl)-1H-indole-2-carboxylate (see intermediate 6, 4.00 g, 7.25 mmol), (4-bromo-5-ethyl-1-methyl-1H-pyrazol-3-yl)methanol (see intermediate 15, 1.98 g, 9.06 mmol) and potassium phosphate (4.62 g, 21.7 mmol) were dissolved in 31 mL of 1,4-dioxane and 10 mL of water and purged with argon for 10 min. XPhos Pd G3 (2.00 g, 2.54 mmol) was added and argon was purged through the reaction mixture for 10 min. The four mixtures were heated for 20 min. to 110° C. in a microwave reactor and were then concentrated under reduced pressure. The crude product was purified by flash chromatography using silica gel (gradient dichloromethane/acetone) to obtain the title compound (1.38 g, 34% yield).

LC-MS (Method 2): Rₜ=1.60 min, MS (ESIpos): m/z=564 [M+H]⁺

¹H-NMR (400 MHz, DMSO-d6) δ [ppm]: 0.844 (3.60), 0.863 (8.25), 0.882 (3.71), 1.065 (6.61), 1.171 (0.44), 1.228 (5.07), 1.246 (10.84), 1.263 (5.03), 2.084 (11.39), 2.195 (1.05), 2.211 (1.45), 2.230 (1.09), 2.246 (0.40), 2.331 (0.93), 2.336 (0.57), 2.357 (0.80), 2.376 (1.54), 2.395 (1.56), 2.399 (1.58), 2.418 (1.49), 2.437 (0.82), 2.456 (0.51), 2.518 (5.41), 2.522 (3.43), 2.673 (0.91), 3.290 (1.24), 3.311 (2.13), 3.700 (0.78), 3.760 (0.48), 3.830 (16.00), 3.939 (1.16), 4.028 (0.76), 4.042 (0.76), 4.058 (1.01), 4.071 (0.97), 4.203 (1.60), 4.217 (4.61), 4.234 (3.85), 4.238 (3.37), 4.251 (3.26), 4.255 (3.01), 4.269 (0.88), 4.273 (0.78), 4.825 (0.78), 4.837 (1.52), 4.848 (0.76), 6.893 (1.26), 6.901 (1.37), 6.906 (1.18), 6.914 (1.35), 7.162 (3.54), 7.184 (3.66), 7.379 (0.80), 7.386 (0.95), 7.402 (1.39), 7.408 (1.49), 7.424 (0.97), 7.430 (1.05), 7.441 (2.82), 7.446 (2.97), 7.455 (5.73), 7.652 (1.54), 7.658 (1.60), 7.678 (1.62), 7.684 (1.71), 7.690 (3.01), 7.711 (2.65), 8.251 (1.37), 8.265 (1.43), 8.273 (1.39), 8.289 (1.33), 10.760 (2.00).

### Intermediate 56

### (rac)-Ethyl 3-chloro-4-ethyl-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylate

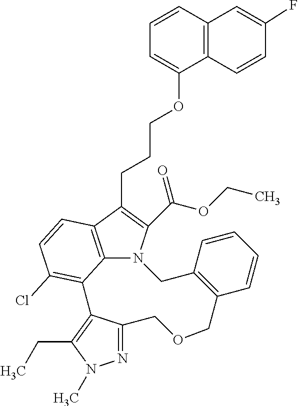 [SMILES: CCOC(=O)c1c(CCCOc2cccc3cc(F)ccc23)c2ccc(Cl)c3c2n1Cc1ccccc1COCc1nn(C)c(CC)c1-3]

Ethyl 6-chloro-7-[5-ethyl-3-(hydroxymethyl)-1-methyl-1H-pyrazol-4-yl]-3-{3-[(6-fluoronaphthalen-1-yl)oxy]propyl}-1H-indole-2-carboxylate (see intermediate 55, 341 mg, 605 μmol) was dissolved in 6.6 mL of THF and 10 mL of acetonitrile. Cesium carbonate (985 mg, 3.02 mmol) was added and the mixture was stirred for 10 minutes at rt. 1,2-Bis(bromomethyl)benzene (CAS 91-13-4, 197 mg, 97% purity, 725 μmol) was added, and the reaction mixture was stirred overnight at 50° C. The mixture was concentrated under reduced pressure. The crude product was purified by flash chromatography using silica gel (gradient dichloromethane/acetone) to give the title compound (379 mg, 94% yield).

LC-MS (Method 2): Rₜ=1.77 min, MS (ESIpos): m/z=666 [M+H]⁺

¹H-NMR (400 MHz, DMSO-d6) δ [ppm]: 0.884 (1.35), 0.903 (3.18), 0.922 (1.37), 1.155 (2.11), 1.173 (4.87), 1.190 (2.15), 2.076 (0.56), 2.084 (16.00), 2.094 (0.66), 2.110 (0.43), 2.235 (0.49), 2.314 (0.48), 2.322 (0.43), 2.327 (0.56), 2.332 (0.62), 2.518 (1.73), 2.523 (1.19), 2.669 (0.51), 3.869 (7.21), 3.886 (0.62), 3.911 (0.78), 4.033 (0.46), 4.057 (0.83), 4.076 (0.97), 4.082 (1.15), 4.108 (1.07), 4.159 (0.56), 4.177 (1.83), 4.195 (1.74), 4.213 (0.50), 4.418 (0.98), 4.450 (0.85), 5.124 (0.55), 5.161 (0.79), 5.285 (0.77), 5.321 (0.59), 5.759 (0.44), 6.735 (0.55), 6.739 (0.57), 6.751 (0.54), 6.756 (0.58), 7.071 (0.48), 7.075 (0.49), 7.080 (0.54), 7.084 (0.67), 7.088 (0.75), 7.093 (0.75), 7.098 (0.69), 7.102 (0.77), 7.164 (0.73), 7.167 (0.64), 7.182 (1.04), 7.187 (0.88), 7.200 (0.61), 7.205 (0.53), 7.271 (1.94), 7.292 (1.80), 7.360 (0.43), 7.375 (0.54), 7.383 (0.59), 7.398 (0.58), 7.405 (0.43), 7.418 (0.83), 7.433 (1.82), 7.640 (0.64), 7.647 (0.65), 7.666 (0.64), 7.673 (0.63), 7.746 (1.69), 7.768 (1.42), 8.190 (0.55), 8.205 (0.58), 8.213 (0.56), 8.228 (0.54).

### Intermediate 57

### Ethyl 6-chloro-7-[3-ethyl-5-(hydroxymethyl)-1-methyl-1H-pyrazol-4-yl]-3-{3-[(6-fluoronaphthalen-1-yl)oxy]propyl}-1H-indole-2-carboxylate

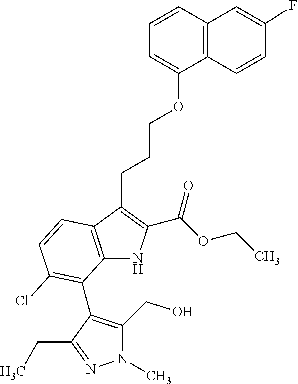 [SMILES: CCOC(=O)c1[nH]c2c(-c3c(CC)nn(C)c3CO)c(Cl)ccc2c1CCCOc1cccc2cc(F)ccc12]

The reaction was performed in four identical preparations using a quarter of all materials.

Ethyl 6-chloro-3-{3-[(6-fluoronaphthalen-1-yl)oxy]propyl}-7-(4,4,5,5-tetramethyl-1,3,2-dioxaborolan-2-yl)-1H-indole-2-carboxylate (see intermediate 6, 4.00 g, 7.25 mmol) and (4-bromo-3-ethyl-1-methyl-1H-pyrazol-5-yl)methanol (see intermediate 19, 1.67 g, 7.61 mmol) were dissolved in 88 mL of THF, and aqueous potassium phosphate solution (29 mL, 0.50 M, 14 mmol) was added. The mixture was purged with argon for 10 min. Subsequently, XPhos Pd G3 (285 mg, 362 μmol) was added and argon was purged through the reaction mixture for 10 min. The four reaction mixtures were heated for 20 min. to 110° C. in a microwave reactor and were then concentrated under reduced pressure. The crude product was purified by flash chromatography using silica gel (gradient dichloromethane/acetone) to obtain the title compound (2.72 g, 66% yield).

LC-MS (Method 2): Rₜ=1.62 min, MS (ESIpos): m/z=564 [M+H]⁺

¹H-NMR (400 MHz, DMSO-d6) δ [ppm]: 0.929 (0.88), 0.948 (2.01), 0.967 (0.89), 1.066 (16.00), 1.232 (0.88), 1.250 (1.94), 1.268 (0.90), 2.083 (1.23), 2.327 (0.41), 2.518 (0.50), 3.879 (2.74), 3.938 (2.61), 4.212 (0.49), 4.242 (0.46), 4.244 (0.52), 4.259 (0.52), 4.262 (0.55), 7.164 (0.69), 7.185 (0.70), 7.438 (0.45), 7.444 (0.46), 7.452 (0.95), 7.700 (0.53), 7.721 (0.48).

### Intermediate 58

### (rac)-Ethyl 3-chloro-4-ethyl-17-{3-[(6-fluoronaphthalen-1-yl)oxy]propyl}-6-methyl-6,7,9,14-tetrahydroindolo[7,1-fg]pyrazolo[3,4-d][2,8]benzoxazacycloundecine-16-carboxylate

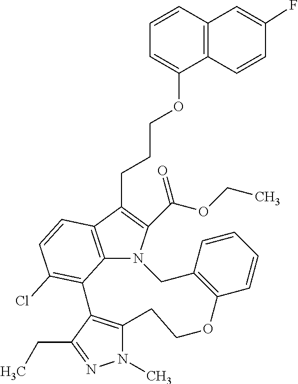 [SMILES: CCOC(=O)c1c(CCCOc2cccc3cc(F)ccc23)c2ccc(Cl)c3c2n1Cc1ccccc1OCCc1c-3c(CC)nn1C]

Ethyl 6-chloro-7-[3-ethyl-5-(hydroxymethyl)-1-methyl-1H-pyrazol-4-yl]-3-{3-[(6-fluoronaphthalen-1-yl)oxy]propyl}-1H-indole-2-carboxylate (see intermediate 57, 500 mg, 886 μmol) was dissolved in 9.7 mL of THF and 15 mL of acetonitrile. Cesium carbonate (1.44 g, 4.43 mmol) was added and the mixture was stirred for 10 minutes at rt. 1,2-Bis(bromomethyl)benzene (CAS 91-13-4, 289 mg, 97% purity, 1.06 mmol) was added and the reaction mixture was stirred overnight at 50° C. The mixture was concentrated under reduced pressure. The crude product was purified by flash chromatography using silica gel (gradient dichloromethane/acetone) to give the title compound (367 mg, 62% yield).

LC-MS (Method 2): Rₜ=1.78 min, MS (ESIpos): m/z=666 [M+H]⁺

¹H-NMR (400 MHz, DMSO-d6) δ [ppm]: 0.850 (3.49), 0.869 (8.12), 0.888 (3.64), 1.150 (3.67), 1.168 (8.20), 1.186 (3.75), 2.057 (0.53), 2.066 (0.49), 2.075 (1.36), 2.084 (16.00), 2.094 (1.32), 2.104 (1.25), 2.113 (0.46), 2.122 (0.50), 2.140 (0.41), 2.159 (0.64), 2.174 (0.86), 2.191 (0.63), 2.332 (0.56), 2.518 (2.68), 2.523 (1.88), 2.673 (0.56), 3.162 (0.44), 3.180 (0.63), 3.240 (0.65), 3.257 (0.44), 3.848 (11.80), 3.876 (1.44), 3.973 (0.82), 4.002 (1.40), 4.065 (1.59), 4.084 (0.52), 4.093 (1.34), 4.107 (0.86), 4.124 (0.65), 4.132 (0.59), 4.141 (1.16), 4.150 (1.04), 4.159 (1.73), 4.169 (1.79), 4.177 (1.72), 4.187 (1.46), 4.195 (0.63), 4.205 (0.41), 4.714 (1.51), 4.748 (1.46), 5.160 (0.67), 5.198 (1.62), 5.239 (1.49), 5.278 (0.68), 6.775 (1.01), 6.795 (2.10), 6.807 (0.99), 6.812 (1.05), 7.091 (0.46), 7.097 (0.50), 7.109 (0.84), 7.114 (0.91), 7.128 (0.64), 7.133 (0.74), 7.150 (0.50), 7.154 (0.52), 7.169 (1.20), 7.172 (1.31), 7.188 (2.45), 7.192 (1.72), 7.206 (0.55), 7.225 (3.09), 7.246 (3.34), 7.350 (0.67), 7.356 (0.75), 7.372 (0.97), 7.379 (1.05), 7.394 (0.70), 7.401 (1.04), 7.423 (1.55), 7.440 (3.18), 7.457 (0.42), 7.644 (1.12), 7.651 (1.14), 7.670 (1.13), 7.677 (1.12), 7.763 (2.92), 7.784 (2.63), 8.212 (0.97), 8.226 (1.02), 8.234 (0.99), 8.250 (0.94).

### Intermediate 59

### Ethyl 7-bromo-3-(3-ethoxy-3-oxo-propyl)-6-fluoro-1H-indole-2-carboxylate

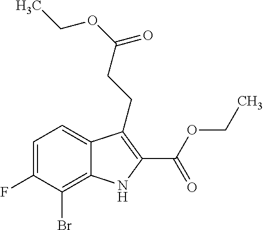 [SMILES: CCOC(=O)CCc1c(C(=O)OCC)[nH]c2c(Br)c(F)ccc12]

To a stirred suspension of 2-bromo-3-fluoroaniline (CAS 111721-75-6, 40.0 g, 210 mmol, 1.00 eq.) in an aqueous hydrochloric acid solution (53.0 mL concentrated hydrochloric acid in 339 mL of water, 630 mmol, 3.00 eq.) was added a 2.5 M solution of sodium nitrite in water (83.9 mL, 210 mmol, 1.00 eq.) via dropping funnel at a temperature of 0° C. After complete addition, 4.5 M sodium acetate (262 mL, 1.18 mol, 5.62 eq.) in water was added via dropping funnel, followed by addition of ethyl 2-oxocyclopentanecarboxylate (CAS 611-10-9, 31.0 mL, 210 mmol, 1.00 eq.). The resulting yellow suspension was maintained at 0° C. for 15 minutes and was then warmed to room temperature and stirred for 2 hours. The reaction mixture was extracted four times with dichloromethane (200 mL each) and the combined organic extracts were dried (magnesium sulfate), filtered and concentrated under reduced pressure to give the crude hydrazone as a orange solid. The residue was re-suspended in ethanol (210 mL, 1.00 M), cooled to 0° C., followed by slow addition of concentrated sulfuric acid (27.9 mL, 525 mmol, 2.50 eq.). The dark red solution was heated to 95° C. for 13 days, cooled to room temperature and partially concentrated under reduced pressure. The dark brown solution was poured onto ice/water (500 mL) and extracted with dichloromethane thrice (500 mL each). The combined organic extracts were washed with saturated aqueous sodium bicarbonate (500 mL), dried (magnesium sulfate), filtered and concentrated under reduced pressure to give a brown solid. The residue was purified by flash column chromatography (20% ethyl acetate/hexanes) and was then recrystallised from hot 10% ethyl acetate/hexanes to give the title compound as a white fluffy solid (40.8 g).

LC-MS (Method 3): Rₜ=1.62 min; MS (ESIpos): m/z=388 [M+H]⁺

¹H NMR (300 MHz, Chloroform-d) δ [ppm]: 8.81 (0.78), 7.63 (1.03), 6.98 (1.02), 4.44 (2.08), 4.08 (2.08), 3.36 (2.07), 2.66 (2.08), 1.44 (3.27), 1.20 (3.61).

### Intermediate 60

### Ethyl 7-bromo-6-fluoro-3-(3-hydroxypropyl)-1H-indole-2-carboxylate

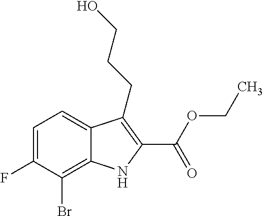 [SMILES: CCOC(=O)c1[nH]c2c(Br)c(F)ccc2c1CCCO]

To a stirred solution of ethyl 7-bromo-3-(3-ethoxy-3-oxopropyl)-6-fluoro-1H-indole-2-carboxylate (see intermediate 59, 24.0 g, 62.1 mmol, 1.00 eq.) in anhydrous tetrahydrofuran (621 mL, 0.10 M) was added borane dimethyl sulfide complex (23.4 mL, 248 mmol, 4.00 eq.) at a temperature of 0° C. The resulting mixture was stirred at 0° C. for 30 minutes and then warmed to room temperature and stirred for 2 days. Methanol was added to the mixture to decompose any remaining borane and the mixture was concentrated three times from methanol. The residue was purified by flash column chromatography (30-100% ethyl acetate/hexanes gradient) to give the title compound as a white solid (19.1 g).

LC-MS (Method 3): Rₜ=1.33 min; MS (ESIpos): m/z=346 [M+H]⁺

¹H NMR (300 MHz, Chloroform-d) δ [ppm]: 1.45 (3.12), 1.93 (2.05), 2.28 (0.95), 3.21 (2.02), 3.57 (2.08), 4.46 (2.04), 6.98 (0.99), 7.59 (1.03), 8.73 (0.72).

### Intermediate 61

### Ethyl-7-bromo-3-(3-((tert-butyldimethylsilyl)oxy)propyl)-6-fluoro-1H-indole-2-carboxylate

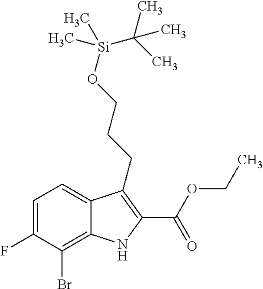 [SMILES: CCOC(=O)c1[nH]c2c(Br)c(F)ccc2c1CCCO[Si](C)(C)C(C)(C)C]

To a stirred solution of ethyl 7-bromo-6-fluoro-3-(3-hydroxypropyl)-1H-indole-2-carboxylate (see intermediate 60, 18.4 g, 53.1 mmol, 1.00 eq.) in anhydrous dichloromethane (265 mL, 0.20 M) was added imidazole (5.41 g, 79.6 mmol, 1.50 eq.) and tert-butylchlorodimethylsilane (CAS 18162-48-6, 9.60 g, 63.7 mmol, 1.20 eq.) at a temperature of 0° C. The resulting mixture was warmed to room temperature and stirred for 30 minutes. The mixture was diluted with water (200 mL) and extracted with dichloromethane thrice (100 mL each). The combined organic extracts were dried (magnesium sulfate), filtered and concentrated under reduced pressure. The residue was purified by flash column chromatography (0-20% ethyl acetate/hexanes gradient) to give the title compound as a white solid (23.2 g).

LC-MS (Method 3): Rₜ=2.21 min, MS (ESIpos): m/z=458 [M+H]⁺.

¹H-NMR (300 MHz, CHLOROFORM-d) δ [ppm]: 8.77 (s, 1H), 7.61 (dd, 1H), 6.96 (t, 1H), 4.43 (q, 2H), 3.65 (t, 2H), 3.12 (m, 2H), 1.86 (m, 2H), 1.43 (t, 3H), 0.92 (s, 9H), 0.05 (s, 6H).

### Intermediate 62

### Ethyl 3-(3-((tert-butyldimethylsilyl)oxy)propyl)-6-fluoro-7-(4,4,5,5-tetramethyl-1,3,2-dioxaborolan-2-yl)-1H-indole-2-carboxylate

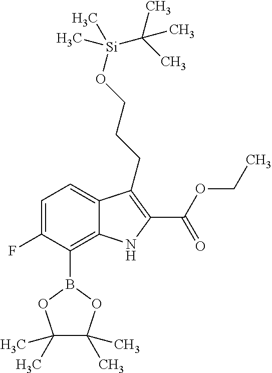 [SMILES: CCOC(=O)c1[nH]c2c(B3OC(C)(C)C(C)(C)O3)c(F)ccc2c1CCCO[Si](C)(C)C(C)(C)C]

To a mixture of ethyl 7-bromo-3-(3-((tert-butyldimethylsilyl)oxy)propyl)-6-fluoro-1H-indole-2-carboxylate (see intermediate 61, 11.0 g, 24.0 mmol, 1.00 eq.), bis(pinacolato)diboron (7.28 g, 28.7 mmol, 1.20 eq.), potassium acetate (4.71 g, 48.0 mmol, 2.00 eq.) and Pd(dppf)Cl₂×CH₂Cl₂ (979 mg, 1.20 mmol, 5.00 mol %) under a nitrogen atmosphere was added 1,4-dioxane degassed with nitrogen (48.0 mL, 0.50 M). The resulting red suspension was heated to 90° C. for 2 days, cooled to room temperature. Celite was added to the suspension, volatiles were removed under reduced pressure, and the residue was subjected to flash chromatography (5-20% ethyl acetate/hexanes gradient) to give the title compound as yellow solid. (12.1 g, >90% pure). The mixture was used directly in the next step.

LC-MS (Method 3): Rₜ=2.35 min, MS (ESIpos): m/z=506 [M+H]⁺.

¹H-NMR (300 MHz, CHLOROFORM-d) δ [ppm]: 9.67 (s, 1H), 7.76 (m, 1H), 6.86 (dd, 1H), 4.41 (q, 2H), 3.65 (t, 2H), 3.12 (m, 2H), 1.86 (m, 2H), 1.42 (d, 14H), 1.26 (s, 3H), 0.92 (s, 9H), 0.05 (s, 6H).

### Intermediate 63

### Ethyl 3-(3-((tert-butyldimethylsilyl)oxy)propyl)-6-fluoro-7-(3-(hydroxymethyl)-1,5-dimethyl-1H-pyrazol-4-yl)-1H-indole-2-carboxylate

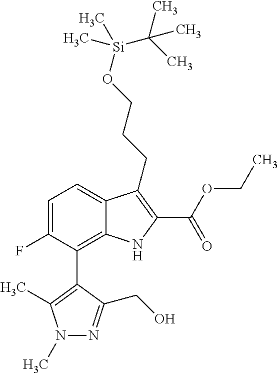 [SMILES: CCOC(=O)c1[nH]c2c(-c3c(CO)nn(C)c3C)c(F)ccc2c1CCCO[Si](C)(C)C(C)(C)C]

To a stirred suspension of (4-bromo-1,5-dimethyl-1H-pyrazol-3-yl)methanol (see intermediate 12, 3.85 g, 18.8 mmol, 1.20 eq.), XPhos Pd G3 (922 mg, 1.09 mmol, 7.00 mol %) and potassium phosphate tribasic (6.64 g, 31.3 mmol, 2.00 eq.) in a 2:1 mixture of 1,4-dioxane/water which had been degassed with argon (45 mL, 0.33 M) was slowly added dropwise (approximately over a period of 1 h) a solution of ethyl 3-(3-((tert-butyldimethylsilyl)oxy)propyl)-6-fluoro-7-(4,4,5,5-tetramethyl-1,3,2-dioxaborolan-2-yl)-1H-indole-2-carboxylate (see intermediate 62, 7.96 g, 15.7 mmol, 1.00 eq.) 1,4-dioxane degassed with argon (16 mL, 1.00 M). The resulting dark mixture was heated to 50° C. for a further 30 min, cooled to room temperature and concentrated under reduced pressure. The residue was dissolved in water (50 mL) and extracted with ethyl acetate thrice (50 mL each). The combined organic extracts were washed with brine (50 mL), dried (magnesium sulfate), filtered and concentrated under reduced pressure. The residue was purified by flash column chromatography (0-100% acetone/dichloromethane gradient) and then reverse phase column chromatography (10-100% acetonitrile/water gradient) to give the title compound as an off-white solid (5.67 g).

LC-MS (Method 3): Rₜ=1.94 min, MS (ESIpos): m/z=502 [M−H]⁻.

¹H-NMR (300 MHz, CHLOROFORM-d) δ [ppm]: 10.12 (s, 1H), 7.65 (dd, 2H), 6.99 (m, 2H), 4.70 (dt, 2H), 4.36 (m, 6H), 3.89 (s, 6H), 3.70 (m, 4H), 3.58 (m, 1H), 3.15 (dd, 4H), 2.17 (d, 6H), 1.92 (m, 4H), 1.37 (m, 6H), 0.93 (d, 19H), 0.07 (d, 12H).

### Intermediate 64

### (rac)-Ethyl-1-(3-((tert-butyldimethylsilyl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate

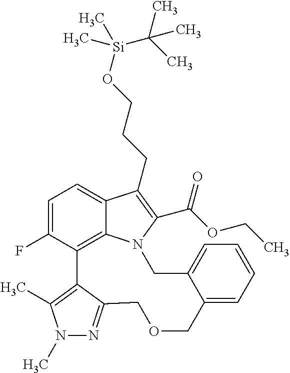 [SMILES: CCOC(=O)c1c(CCCO[Si](C)(C)C(C)(C)C)c2ccc(F)c3c2n1Cc1ccccc1COCc1nn(C)c(C)c1-3]

To stirred solution of ethyl 3-(3-((tert-butyldimethylsilyl)oxy)propyl)-6-fluoro-7-(3-(hydroxymethyl)-1,5-dimethyl-1H-pyrazol-4-yl)-1H-indole-2-carboxylate (see intermediate 63, 3.59 g, 7.12 mmol, 1.00 eq.) in a 2:1 mixture of acetonitrile/tetrahydrofuran (356 mL, 0.02 M) was added cesium carbonate (11.5 g, 35.6 mmol, 5.00 eq.) in one portion at room temperature. After stirring for a further 10 min, 1,2-bis(bromomethyl)benzene (CAS 91-13-4, 2.06 g, 7.83 mmol, 1.10 eq.) was added and the resulting mixture was then heated to 80° C. for 2 days. The mixture was cooled to room temperature and filtered through a pad of Celite. The filter cake was washed with ethyl acetate, and the combined filtrates were and concentrated under reduced pressure. The residue was purified by flash column chromatography (0-15% acetone/dichloromethane gradient) to give the title compound as a white solid (1.03 g).

LC-MS (Method 3): Rₜ=5.85 min, MS (ESIpos): m/z=606 [M+H]⁺.

¹H-NMR (300 MHz, CHLOROFORM-d) δ [ppm]: 7.60 (dd, 1H), 7.13 (dt, 4H), 6.95 (t, 1H), 5.48 (d, 1H), 5.31 (d, 1H), 4.62 (d, 1H), 4.26 (m, 5H), 3.87 (s, 3H), 3.60 (td, 2H), 2.96 (m, 2H), 2.00 (s, 3H), 1.80 (m, 2H), 1.32 (t, 3H), 0.90 (s, 9H), 0.03 (s, 7H).

### Intermediate 65

### (rac)-Ethyl 15-fluoro-1-(3-hydroxypropyl)-13,14-dimethyl-4,9,11,13-tetrahydrobenzo[3,4]pyrazolo[4′,3′:9,10][1,6]oxaazacycloundecino[8,7,6-hi]indole-2-carboxylate

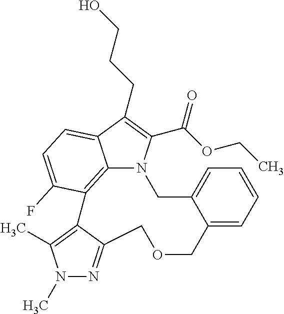 [SMILES: CCOC(=O)c1c(CCCO)c2ccc(F)c3c2n1Cc1ccccc1COCc1nn(C)c(C)c1-3]

To a stirred solution of (rac)-ethyl 1-(3-((tert-butyldimethylsilyl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 64, 1.03 g, 1.70 mmol, 1.00 eq.) in anhydrous tetrahydrofuran (33.9 mL, 0.05 M) was added a 1.0 M solution of tetrabutylammonium fluoride in tetrahydrofuran (2.03 mL, 2.03 mmol, 1.20 eq.) at a temperature of 0° C. The resulting light yellow mixture was warmed to room temperature stirred for 2 hours and was then concentrated under reduced pressure. The residue was resuspended in saturated aqueous ammonium chloride (20 mL) and extracted with ethyl acetate thrice (20 mL each). The combined organic extracts were washed with brine (20 mL), dried (magnesium sulfate), filtered. Celite was added to the resulting solution, volatiles were removed under reduced pressure, and the residue was loaded onto a silica gel cartridge which was subjected to flash chromatography (0-30% acetone/dichloromethane gradient) to give the title compound as a white solid (821 mg).

LC-MS (Method 3): Rₜ=1.31 min, MS (ESIpos): m/z=492 [M+H]⁺.

¹H-NMR (300 MHz, CHLOROFORM-d) δ [ppm]: 7.56 (dd, 1H), 7.13 (m, 4H), 6.96 (t, 1H), 5.49 (d, 1H), 5.26 (d, 1H), 4.61 (d, 1H), 4.36 (m, 3H), 4.25 (d, 1H), 4.13 (d, 1H), 3.88 (s, 3H), 3.46 (m, 2H), 3.05 (m, 2H), 2.32 (s, 1H), 2.01 (s, 5H), 1.36 (t, 3H).

### Intermediate 66

### (rac)-Ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate

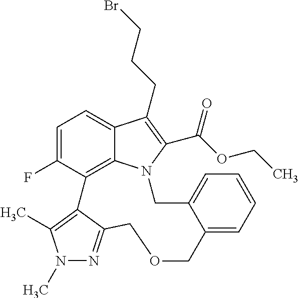 [SMILES: CCOC(=O)c1c(CCCBr)c2ccc(F)c3c2n1Cc1ccccc1COCc1nn(C)c(C)c1-3]

To a stirred solution of (rac)-ethyl 15-fluoro-1-(3-hydroxypropyl)-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 65, 820 mg, 1.66 mmol, 1.00 eq.) and triphenylphosphane (477 mg, 1.82 mmol, 1.10 eq.) in anhydrous dichloromethane (16.6 mL, 0.10 M) was added carbon tetrabromide (603 mg, 1.82 mmol, 1.10 eq.) in one portion at a temperature of 0° C. The resulting mixture was warmed to room temperature, stirred for 1 hour and was then concentrated under reduced pressure. Celite was added to the residue, volatiles were removed under reduced pressure, and the residue was loaded onto a silica gel cartridge which was subjected to flash chromatography (0-15% acetone/dichloromethane gradient) to give the title compound as a white solid (743 mg).

LC-MS (Method 3): Rₜ=1.72 min, MS (ESIpos): m/z=554 [M+H]⁺.

¹H-NMR (300 MHz, CHLOROFORM-d) δ [ppm]: 7.60 (dd, 1H), 7.13 (ddq, 4H), 6.98 (t, 1H), 5.50 (d, 1H), 5.32 (d, 1H), 4.62 (d, 1H), 4.32 (m, 3H), 4.23 (d, 1H), 4.14 (d, 1H), 3.87 (s, 3H), 3.38 (qt, 2H), 3.07 (m, 2H), 2.16 (p, 2H), 2.01 (s, 3H), 1.33 (t, 3H).

### Intermediate 67

### (rac)-Ethyl 1-(3-(4-chloro-3,5-dimethylphenoxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate

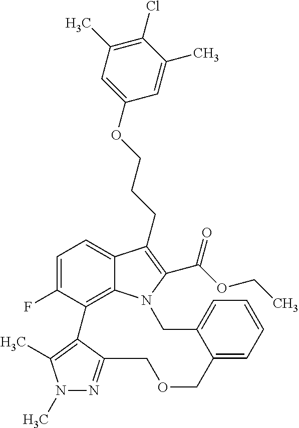 [SMILES: CCOC(=O)c1c(CCCOc2cc(C)c(Cl)c(C)c2)c2ccc(F)c3c2n1Cc1ccccc1COCc1nn(C)c(C)c1-3]

To a stirred solution of (rac)-ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 66, 55.4 mg, 0.10 mmol, 1.00 eq.) in anhydrous tetrahydrofuran (1.00 mL, 0.10 M) were added cesium carbonate (195 mg, 0.60 mmol, 6.00 eq.) and 4-chloro-3,5-dimethylphenol (CAS 88-04-0, 31.3 mg, 0.20 mmol, 2.00 eq.). The resulting suspension was heated to 55° C. for 13 hours and cooled to room temperature. Celite was added to the resulting solution, volatiles were removed under reduced pressure, and the residue was loaded onto a silica gel cartridge which was subjected to reverse phase column chromatography (10-100% acetonitrile/water with 0.1% formic acid gradient) to give the title compound as a foamy solid (61.0 mg).

LC-MS (Method 3): Rₜ=2.04 min, MS (ESIpos): m/z=630 [M+H]⁺.

¹H-NMR (300 MHz, CHLOROFORM-d) δ [ppm]: 7.56 (dd, 1H), 7.13 (dt, 4H), 6.94 (t, 1H), 6.57 (s, 2H), 5.49 (d, 1H), 5.32 (d, 1H), 4.62 (d, 1H), 4.25 (m, 5H), 3.86 (m, 5H), 3.10 (ddt, 2H), 2.32 (s, 6H), 2.01 (s, 5H), 1.31 (t, 3H).

### Intermediate 68

### (rac)-Ethyl 1-(3-((2,3-dihydro-1H-inden-4-yl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate

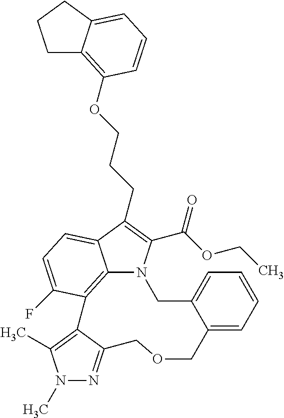 [SMILES: CCOC(=O)c1c(CCCOc2cccc3c2CCC3)c2ccc(F)c3c2n1Cc1ccccc1COCc1nn(C)c(C)c1-3]

To a stirred solution of (rac)-ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 66, 55.4 mg, 0.10 mmol, 1.00 eq.) in anhydrous tetrahydrofuran (1.00 mL, 0.10 M) were added cesium carbonate (195 mg, 0.60 mmol, 6.00 eq.) and 4-indanol (CAS 1641-41-4, 26.8 mg, 0.20 mmol, 2.00 eq.). The resulting suspension was heated to 55° C. for 13 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a foamy solid (59.8 mg).

LC-MS (Method 3): Rₜ=2.00 min, MS (ESIpos): m/z=608 [M+H]⁺.

¹H-NMR (300 MHz, CHLOROFORM-d) δ [ppm]: 7.58 (dd, 1H), 7.13 (d, 3H), 7.07 (t, 1H), 6.94 (t, 1H), 6.84 (d, 1H), 6.54 (d, 1H), 5.50 (d, 1H), 5.32 (d, 1H), 4.62 (d, 1H), 4.26 (m, 5H), 3.88 (s, 4H), 3.18 (dt, 1H), 3.06 (m, 1H), 2.91 (q, 4H), 2.03 (m, 6H), 1.30 (t, 3H).

### Intermediate 69

### (rac)-Ethyl 15-fluoro-13,14-dimethyl-1-(3-((5,6,7,8-tetrahydronaphthalen-1-yl)oxy)propyl)-4,9,11,13-tetrahydrobenzo[3,4]pyrazolo[4′,3′:9,10][1,6]oxaazacycloundecino[8,7,6-hi]indole-2-carboxylate

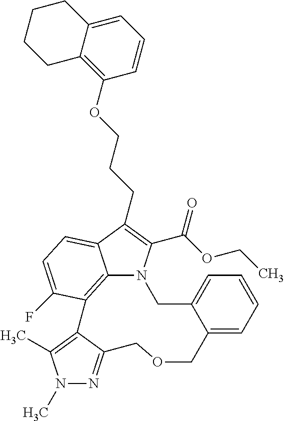 [SMILES: CCOC(=O)c1c(CCCOc2cccc3c2CCCC3)c2ccc(F)c3c2n1Cc1ccccc1COCc1nn(C)c(C)c1-3]

To a stirred solution of (rac)-ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 66, 55.4 mg, 0.10 mmol, 1.00 eq.) in anhydrous tetrahydrofuran (1.00 mL, 0.10 M) were added cesium carbonate (195 mg, 0.60 mmol, 6.00 eq.) and 5,6,7,8-tetrahydronaphthalen-1-ol (CAS 529-35-1, 29.6 mg, 0.20 mmol, 2.00 eq.). The resulting suspension was heated to 55° C. for 13 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a foamy solid (53.8 mg).

LC-MS (Method 3): Rₜ=2.08 min, MS (ESIpos): m/z=622 [M+H]⁺.

¹H-NMR (300 MHz, CHLOROFORM-d) δ [ppm]: 7.57 (dd, 1H), 7.13 (m, 4H), 7.02 (t, 1H), 6.94 (t, 1H), 6.69 (d, 1H), 6.53 (d, 1H), 5.50 (d, 1H), 5.32 (d, 1H), 4.62 (d, 1H), 4.26 (m, 5H), 3.88 (s, 4H), 3.19 (dt, 1H), 3.06 (m, 1H), 2.72 (dd, 4H), 2.01 (d, 7H), 1.78 (dd, 3H), 1.29 (t, 3H).

### Intermediate 70

### (rac)-Ethyl 15-fluoro-1-(3-((4-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylate

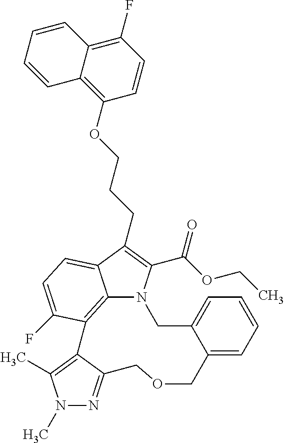 [SMILES: CCOC(=O)c1c(CCCOc2ccc(F)c3ccccc23)c2ccc(F)c3c2n1Cc1ccccc1COCc1nn(C)c(C)c1-3]

To a stirred solution of (rac)-ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 66, 138 mg, 0.25 mmol, 1.00 eq.) in anhydrous tetrahydrofuran (2.50 mL, 0.10 M) were added cesium carbonate (488 mg, 1.50 mmol, 6.00 eq.) and 4-fluoronaphthalen-1-ol (CAS 315-53-7, 81.0 mg, 0.50 mmol, 2.00 eq.). The resulting suspension was heated to 55° C. for 13 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a foamy solid (152 mg).

LC-MS (Method 3): Rₜ=1.99 min, MS (ESIpos): m/z=636 [M+H]⁺.

¹H-NMR (300 MHz, CHLOROFORM-d) δ [ppm]: 8.28 (dt, 1H), 8.04 (m, 1H), 7.56 (m, 3H), 7.13 (m, 4H), 6.95 (m, 2H), 6.53 (dd, 1H), 5.51 (d, 1H), 5.32 (d, 1H), 4.62 (d, 1H), 4.24 (m, 5H), 4.04 (d, 1H), 3.87 (s, 3H), 3.27 (m, 1H), 3.16 (m, 1H), 2.22 (t, 2H), 2.01 (d, 6H), 1.26 (t, 3H).

### Intermediate 71

### (rac)-Ethyl 15-fluoro-1-(3-((6-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylate

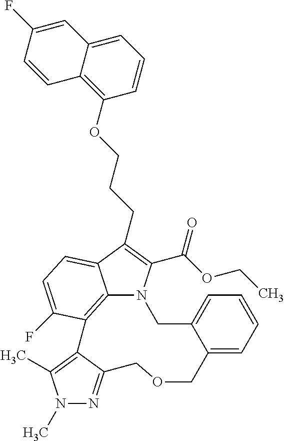 [SMILES: CCOC(=O)c1c(CCCOc2cccc3cc(F)ccc23)c2ccc(F)c3c2n1Cc1ccccc1COCc1nn(C)c(C)c1-3]

To a stirred solution of (rac)-ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 66, 138 mg, 0.25 mmol, 1.00 eq.) in anhydrous tetrahydrofuran (2.50 mL, 0.10 M) were added cesium carbonate (488 mg, 1.50 mmol, 6.00 eq.) and 6-fluoronaphthalen-1-ol (CAS 804498-72-4, 81.0 mg, 0.50 mmol, 2.00 eq.). The resulting suspension was heated to 55° C. for 18 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a yellow solid (156 mg).

LC-MS (Method 3): Rₜ=1.98 min, MS (ESIpos): m/z=636 [M+H]⁺.

¹H-NMR (300 MHz, CHLOROFORM-d) δ [ppm]: 8.30 (dd, 1H), 7.57 (dd, 1H), 7.37 (m, 3H), 7.22 (m, 1H), 7.13 (t, 4H), 6.90 (t, 1H), 6.61 (dd, 1H), 5.51 (d, 1H), 5.32 (d, 1H), 4.62 (d, 1H), 4.20 (m, 7H), 3.88 (s, 3H), 3.28 (dt, 1H), 3.15 (dt, 1H), 2.23 (t, 2H), 2.01 (s, 3H), 1.25 (t, 3H).

### Intermediate 72

### (rac)-Ethyl 15-fluoro-13,14-dimethyl-1-(3-(naphthalen-1-yloxy)propyl)-4,9,11,13-tetrahydrobenzo[3,4]pyrazolo[4′,3′:9,10][1,6]oxaazacycloundecino[8,7,6-hi]indole-2-carboxylate

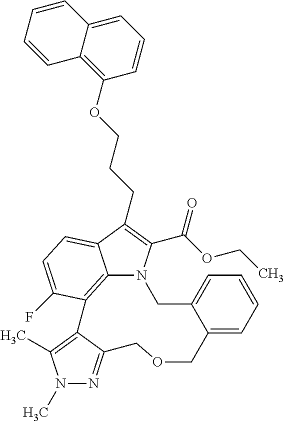 [SMILES: CCOC(=O)c1c(CCCOc2cccc3ccccc23)c2ccc(F)c3c2n1Cc1ccccc1COCc1nn(C)c(C)c1-3]

To a stirred solution of (rac)-ethyl 1-(3-bromopropyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 66, 138 mg, 0.25 mmol, 1.00 eq.) in anhydrous tetrahydrofuran (2.50 mL, 0.10 M) were added cesium carbonate (488 mg, 1.50 mmol, 6.00 eq.) and naphthalen-1-ol (CAS 90-15-3, 72.0 mg, 0.50 mmol, 2.00 eq.). The resulting suspension was heated to 55° C. for 18 hours and cooled to room temperature. Celite was added to the solution, volatiles were removed under reduced pressure, and the residue was loaded onto a silica gel cartridge which was subjected to reverse phase column chromatography (C18, 10-100% acetonitrile/water with 0.1% formic acid gradient) to give the title compound as a yellow solid (152 mg).

LC-MS (Method 3): Rₜ=1.97 min, MS (ESIpos): m/z=618 [M+H]⁺.

¹H-NMR (300 MHz, CHLOROFORM-d) δ [ppm]: 8.32 (m, 1H), 7.80 (m, 1H), 7.47 (m, 5H), 7.13 (d, 4H), 6.90 (t, 1H), 6.67 (dd, 1H), 5.51 (d, 1H), 5.32 (d, 1H), 4.62 (d, 1H), 4.20 (m, 7H), 3.88 (s, 3H), 3.30 (dt, 1H), 3.16 (dt, 1H), 2.24 (p, 2H), 2.01 (d, 3H), 1.25 (t, 3H).

### Intermediate 73

### Ethyl 3-(3-((tert-butyldimethylsilyl)oxy)propyl)-7-(5-ethyl-3-(hydroxymethyl)-1-methyl-1H-pyrazol-4-yl)-6-fluoro-1H-indole-2-carboxylate

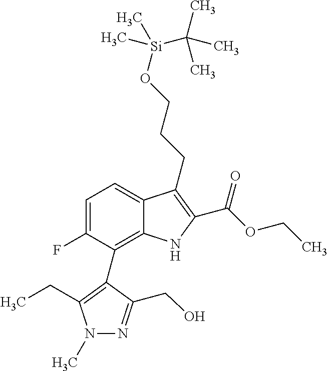 [SMILES: CCOC(=O)c1[nH]c2c(-c3c(CO)nn(C)c3CC)c(F)ccc2c1CCCO[Si](C)(C)C(C)(C)C]

To a stirred suspension of (4-bromo-5-ethyl-1-methyl-1H-pyrazol-3-yl)methanol (see intermediate 15, 3.33 g, 15.2 mmol, 1.00 eq.), XPhos Pd G3 (752 mg, 0.89 mmol, 7.00 mol %) and potassium phosphate tribasic (5.39 g, 25.4 mmol, 2.00 eq.) in a 2:1 mixture of 1,4-dioxane/water degassed with argon (38.1 mL) was slowly added dropwise (over a period of approximately 1 h) a solution of ethyl 3-(3-((tert-butyldimethylsilyl)oxy)propyl)-6-fluoro-7-(4,4,5,5-tetramethyl-1,3,2-dioxaborolan-2-yl)-1H-indole-2-carboxylate (see intermediate 62, 6.42 g, 12.7 mmol, 1.00 eq.) in 1,4-dioxane degassed with argon (12.7 mL, 1.00 M) at a temperature of 50° C. The resulting dark mixture was heated to 50° C. for a further 30 minutes, cooled to room temperature and concentrated under reduced pressure. The residue was dissolved in water (50 mL) and extracted with ethyl acetate thrice (50 mL each). The combined organic extracts were washed with brine (50 mL), dried (magnesium sulfate), and were filtered and concentrated under reduced pressure. The residue was purified by flash column chromatography (0-100% acetone/dichloromethane gradient) and then reverse phase column chromatography (10-100% acetonitrile/water gradient) to give the title compound as a light yellow solid (3.37 g).

LC-MS (Method 3): Rₜ=1.99 min, MS (ESIpos): m/z=518 [M+H]⁺.

¹H-NMR (300 MHz, CHLOROFORM-d) δ [ppm]: 9.60 (s, 1H), 7.64 (dd, 1H), 6.97 (dd, 1H), 4.63 (d, 1H), 4.36 (m, 3H), 3.92 (s, 3H), 3.70 (t, 2H), 3.15 (m, 2H), 2.57 (ddt, 2H), 1.91 (m, 2H), 1.37 (t, 3H), 1.08 (t, 3H), 0.93 (s, 9H), 0.07 (s, 6H)

### Intermediate 74

### (rac)-Ethyl 1-(3-((tert-butyldimethylsilyl)oxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate

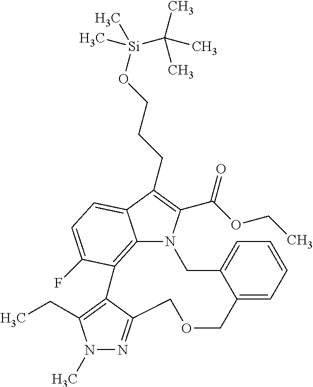 [SMILES: CCOC(=O)c1c(CCCO[Si](C)(C)C(C)(C)C)c2ccc(F)c3c2n1Cc1ccccc1COCc1nn(C)c(CC)c1-3]

To a stirred solution of ethyl 3-(3-((tert-butyldimethylsilyl)oxy)propyl)-7-(5-ethyl-3-(hydroxymethyl)-1-methyl-1H-pyrazol-4-yl)-6-fluoro-1H-indole-2-carboxylate (see intermediate 73, 1.40 g, 2.70 mmol, 1.00 eq.) in a 2:1 mixture of acetonitrile/tetrahydrofuran (135 mL, 0.02 M) was added cesium carbonate (4.39 g, 13.5 mmol, 5.00 eq.) in one portion at room temperature. After stirring for a further 10 minutes, 1,2-bis(bromomethyl)benzene (CAS 91-13-4, 783 mg, 2.97 mmol, 1.10 eq.) was added and the resulting yellow mixture was then heated to 80° C. for 18 hours. The mixture was cooled to room temperature and was filtered through a pad of Celite. The filter cake was washed with ethyl acetate, and the combined filtrates were concentrated under reduced pressure. The residue was purified by flash column chromatography (0-15% acetone/dichloromethane gradient) to give the title compound as a white solid (1.06 g).

LC-MS (Method 3): Rₜ=2.18 min, MS (ESIpos): m/z=620 [M+H]⁺.

¹H-NMR (300 MHz, CHLOROFORM-d) δ [ppm]: 7.60 (dd, 1H), 7.13 (d, 4H), 6.95 (t, 1H), 5.50 (d, 1H), 5.29 (d, 1H), 4.62 (d, 1H), 4.29 (m, 3H), 4.19 (m, 2H), 3.90 (s, 3H), 3.59 (m, 2H), 3.03 (ddd, 1H), 2.89 (ddd, 1H), 2.39 (qd, 2H), 1.79 (m, 2H), 1.31 (t, 3H), 1.03 (t, 3H), 0.90 (s, 9H), 0.02 (s, 6H).

### Intermediate 75

### (rac)-Ethyl 14-ethyl-15-fluoro-1-(3-hydroxypropyl)-13-methyl-4,9,11,13-tetrahydrobenzo[3,4]pyrazolo[4′,3′:9,10][1]oxa[6]azacycloundecino[8,7,6-hi]indole-2-carboxylate

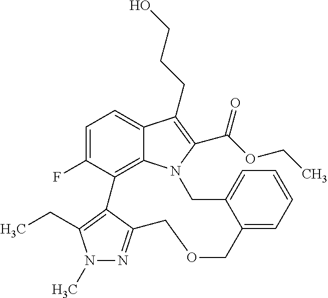 [SMILES: CCOC(=O)c1c(CCCO)c2ccc(F)c3c2n1Cc1ccccc1COCc1nn(C)c(CC)c1-3]

To a stirred solution of (rac)-ethyl 1-(3-((tert-butyldimethylsilyl)oxy)propyl)-14-ethyl-15-fluoro-13-methyl-4,9,11,13-tetrahydrobenzo[3,4]pyrazolo[4′,3′:9,10][1]oxa[6]azacycloundecino-[8,7,6-hi]indole-2-carboxylate (see intermediate 74, 1.02 g, 1.64 mmol, 1.00 eq.) in anhydrous tetrahydrofuran (32.8 mL, 0.05 M) was added a 1.0 M solution of tetrabutylammonium fluoride in tetrahydrofuran (1.96 mL, 1.96 mmol, 1.20 eq.) at a temperature of 0° C. The resulting light yellow mixture was warmed to room temperature, stirred for 3 hours, and then concentrated under reduced pressure. The residue was resuspended in saturated aqueous ammonium chloride solution (20 mL) and was extracted with ethyl acetate thrice (20 mL each). The combined organic extracts were washed with brine (20 mL), dried (magnesium sulfate), filtered and concentrated under reduced pressure. The residue was purified by flash column chromatography (0-40% acetone/dichloromethane gradient) to give the title compound as a white solid (808 mg).

LC-MS (Method 3): Rₜ=1.37 min, MS (ESIpos): m/z=506 [M+H]⁺.

¹H-NMR (300 MHz, CHLOROFORM-d) δ [ppm]: 7.56 (dd, 1H), 7.13 (m, 4H), 6.96 (t, 1H), 5.50 (d, 1H), 5.22 (d, 1H), 4.61 (d, 1H), 4.35 (m, 3H), 4.25 (d, 1H), 4.11 (d, 1H), 3.91 (s, 3H), 3.43 (m, 2H), 3.05 (m, 2H), 2.39 (m, 3H), 1.93 (m, 2H), 1.35 (t, 3H), 1.04 (t, 3H).

### Intermediate 76

### (rac)-Ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate

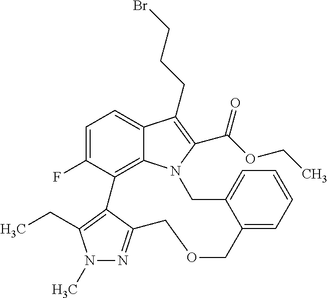 [SMILES: CCOC(=O)c1c(CCCBr)c2ccc(F)c3c2n1Cc1ccccc1COCc1nn(C)c(CC)c1-3]

To a stirred solution of (rac)-ethyl 14-ethyl-15-fluoro-1-(3-hydroxypropyl)-13-methyl-4,9,11,13-tetrahydrobenzo[3,4]pyrazolo[4′,3′:9,10][1]oxa[6]azacycloundecino[8,7,6-hi]indole-2-carboxylate (see Intermediate 75, 805 mg, 1.59 mmol, 1.00 eq.) and triphenylphosphane (456 mg, 1.74 mmol, 1.10 eq.) in anhydrous dichloromethane (15.9 mL, 0.10 M) was added carbon tetrabromide (577 mg, 1.74 mmol, 1.10 eq.) in one portion at a temperature of 0° C. The resulting mixture was warmed to room temperature, stirred for 2 hours and then concentrated under reduced pressure. Celite was added to the residue, volatiles were removed under reduced pressure, and the residue was loaded onto a silica gel cartridge which was subjected to flash column chromatography (0-20% acetone/dichloromethane gradient) to give the title compound as a white solid (800 mg).

LC-MS (Method 3): Rₜ=1.79 min, MS (ESIpos): m/z=568 [M+H]⁺.

¹H-NMR (300 MHz, CHLOROFORM-d) δ [ppm]: 7.61 (dd, 1H), 7.15 (d, 4H), 6.99 (t, 1H), 5.52 (d, 1H), 5.30 (d, 1H), 4.63 (d, 1H), 4.33 (m, 3H), 4.24 (d, 1H), 4.13 (d, 1H), 3.92 (s, 3H), 3.38 (m, 2H), 3.09 (m, 2H), 2.40 (m, 2H), 2.16 (m, 2H), 1.34 (t, 4H), 1.04 (t, 3H).

### Intermediate 77

### (rac)-Ethyl 14-ethyl-15-fluoro-13-methyl-1-(3-(naphthalen-1-yloxy)propyl)-4,9,11,13-tetrahydrobenzo[3,4]pyrazolo[4′,3′:9,10][1]oxa[6]azacycloundecino[8,7,6-hi]indole-2-carboxylate

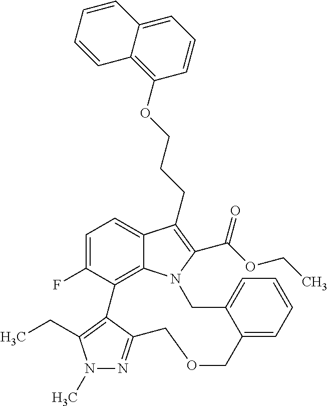 [SMILES: CCOC(=O)c1c(CCCOc2cccc3ccccc23)c2ccc(F)c3c2n1Cc1ccccc1COCc1nn(C)c(CC)c1-3]

To a stirred solution of (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 142 mg, 0.25 mmol, 1.00 eq.) in anhydrous tetrahydrofuran (2.50 mL, 0.10 M) were added cesium carbonate (488 mg, 1.50 mmol, 6.00 eq.) and naphthalen-1-ol (CAS 90-15-3, 72.0 mg, 0.50 mmol, 2.00 eq.). The resulting suspension was heated to 55° C. for 21 hours and cooled to room temperature. Celite was added to the solution, volatiles were removed under reduced pressure, and the residue was loaded onto a silica gel cartridge which was subjected to flash column chromatography (0-20% acetone/dichloromethane gradient) to give the title compound as a tan solid (149 mg).

LC-MS (Method 3): Rₜ=2.02 min, MS (ESIpos): m/z=632 [M+H]⁺.

¹H-NMR (300 MHz, CHLOROFORM-d) δ [ppm]: 8.30 (dd, 1H), 7.57 (dd, 1H), 7.37 (m, 3H), 7.22 (m, 1H), 7.14 (s, 4H), 6.90 (m, 1H), 6.60 (dd, 1H), 5.53 (d, 1H), 5.29 (d, 1H), 4.62 (d, 1H), 4.22 (m, 7H), 3.91 (s, 3H), 3.21 (m, 2H), 2.39 (m, 2H), 2.23 (m, 2H), 1.42 (s, 2H), 1.25 (t, 3H), 1.03 (t, 3H).

### Intermediate 78

### (rac)-Ethyl 14-ethyl-15-fluoro-1-(3-((6-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylate

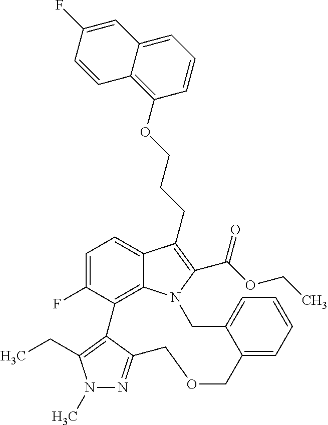 [SMILES: CCOC(=O)c1c(CCCOc2cccc3cc(F)ccc23)c2ccc(F)c3c2n1Cc1ccccc1COCc1nn(C)c(CC)c1-3]

To a stirred solution of (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 142 mg, 0.25 mmol, 1.00 eq.) in anhydrous tetrahydrofuran (2.50 mL, 0.10 M) were added cesium carbonate (488 mg, 1.50 mmol, 6.00 eq.) and 6-fluoronaphthalen-1-ol (CAS 804498-72-4, 81.0 mg, 0.50 mmol, 2.00 eq.). The resulting suspension was heated to 55° C. for 21 hours and cooled to room temperature. Celite was added to the solution, volatiles were removed under reduced pressure, and the residue was loaded onto a silica gel cartridge which was subjected to flash column chromatography (0-20% acetone/dichloromethane gradient) to give the title compound as a tan solid (159 mg).

LC-MS (Method 3): Rₜ=2.02 min, MS (ESIpos): m/z=650 [M+H]⁺.

¹H-NMR (300 MHz, CHLOROFORM-d) δ [ppm]: 8.30 (dd, 1H), 7.57 (dd, 1H), 7.37 (m, 3H), 7.22 (m, 1H), 7.14 (s, 4H), 6.90 (m, 1H), 6.60 (dd, 1H), 5.53 (d, 1H), 5.29 (d, 1H), 4.62 (d, 1H), 4.22 (m, 7H), 3.91 (s, 3H), 3.21 (m, 2H), 2.39 (m, 2H), 2.23 (m, 2H), 1.42 (s, 2H), 1.25 (t, 3H), 1.03 (t, 3H).

### Intermediate 79

### (rac)-Ethyl 14-ethyl-15-fluoro-1-(3-((4-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylate

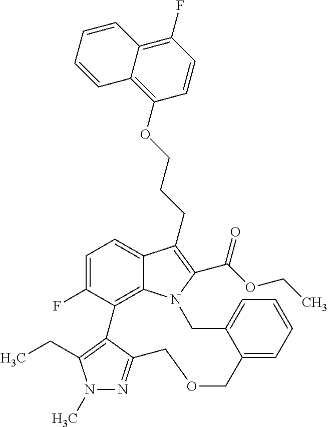 [SMILES: CCOC(=O)c1c(CCCOc2ccc(F)c3ccccc23)c2ccc(F)c3c2n1Cc1ccccc1COCc1nn(C)c(CC)c1-3]

To a stirred solution of (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 142 mg, 0.25 mmol, 1.00 eq.) in anhydrous tetrahydrofuran (2.50 mL, 0.10 M) were added cesium carbonate (488 mg, 1.50 mmol, 6.00 eq.) and 4-fluoronaphthalen-1-ol (CAS 315-53-7, 81.0 mg, 0.50 mmol, 2.00 eq.). The resulting suspension was heated to 55° C. for 2 days and cooled to room temperature. Celite was added to the solution, volatiles were removed under reduced pressure, and the residue was loaded onto a silica gel cartridge which was subjected to flash column chromatography (0-20% acetone/dichloromethane gradient) to the title compound as a tan solid (126 mg).

LC-MS (Method 3): Rₜ=2.04 min, MS (ESIpos): m/z=650 [M+H]⁺.

¹H-NMR (300 MHz, CHLOROFORM-d) δ [ppm]: 8.28 (dt, 1H), 8.04 (m, 1H), 7.56 (m, 3H), 7.14 (s, 4H), 6.95 (m, 2H), 6.51 (dd, 1H), 5.53 (d, 1H), 5.29 (d, 1H), 4.62 (d, 1H), 4.22 (m, 5H), 4.02 (tt, 2H), 3.91 (s, 3H), 3.27 (dt, 1H), 3.15 (dt, 1H), 2.39 (m, 2H), 2.22 (m, 2H), 1.26 (t, 3H), 1.03 (t, 3H).

### Intermediate 80

### (rac)-Ethyl 14-ethyl-15-fluoro-13-methyl-1-(3-((5,6,7,8-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylate

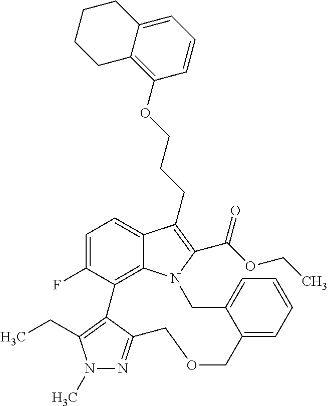 [SMILES: CCOC(=O)c1c(CCCOc2cccc3c2CCCC3)c2ccc(F)c3c2n1Cc1ccccc1COCc1nn(C)c(CC)c1-3]

To a stirred solution of (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 56.8 mg, 0.10 mmol, 1.00 eq.) in anhydrous tetrahydrofuran (1.00 mL, 0.10 M) were added cesium carbonate (195 mg, 0.60 mmol, 6.00 eq.) and 5,6,7,8-tetrahydronaphthalen-1-ol (CAS 529-35-1, 29.6 mg, 0.20 mmol, 2.00 eq.). The resulting suspension was heated to 55° C. for 17 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a light brown solid (62.9 mg).

LC-MS (Method 3): Rₜ=2.12 min, MS (ESIpos): m/z=636 [M+H]⁺.

¹H-NMR (300 MHz, CHLOROFORM-d) 7.62 (dd, 1H), 7.15 (m, 4H), 6.98 (m, 2H), 6.53 (d, 1H), 5.41 (m, 2H), 4.73 (d, 1H), 4.27 (m, 5H), 3.95 (m, 5H), 3.12 (m, 2H), 2.74 (m, 4H), 2.42 (q, 2H), 2.07 (m, 2H), 1.78 (m, 4H), 1.29 (m, 4H), 1.05 (t, 3H).

### Intermediate 81

### (rac)-Ethyl 14-ethyl-15-fluoro-13-methyl-1-(3-((1,2,3,4-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylate

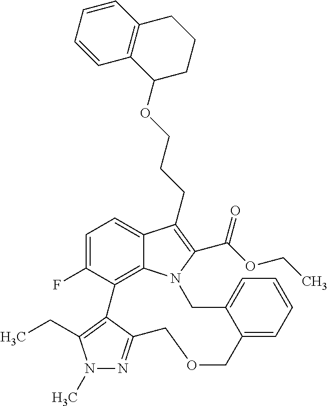 [SMILES: CCOC(=O)c1c(CCCOC2CCCc3ccccc32)c2ccc(F)c3c2n1Cc1ccccc1COCc1nn(C)c(CC)c1-3]

To a stirred solution of 1,2,3,4-tetrahydronaphthalen-1-ol (CAS 529-33-9, 29.6 mg, 0.20 mmol, 2.00 eq.) in anhydrous tetrahydrofuran (1.00 mL, 0.20 M) was added sodium hydride (60%, 6.00 mg, 0.15 mmol, 1.50 eq.). After stirring for 10 minutes, (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 56.8 mg, 0.10 mmol, 1.00 eq.) was added in one portion and the resulting suspension was heated to 55° C. for 17 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a light brown solid (13.9 mg).

LC-MS (Method 3): Rₜ=2.05 min, MS (ESIpos): m/z=636 [M+H]⁺.

### Intermediate 82

### (rac)-Ethyl 1-(3-((2,3-dihydro-1H-inden-4-yl)oxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate

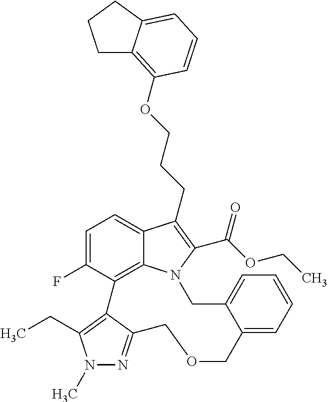 [SMILES: CCOC(=O)c1c(CCCOc2cccc3c2CCC3)c2ccc(F)c3c2n1Cc1ccccc1COCc1nn(C)c(CC)c1-3]

To a stirred solution of (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 56.8 mg, 0.10 mmol, 1.00 eq.) in anhydrous tetrahydrofuran (1.00 mL, 0.10 M) were added cesium carbonate (195 mg, 0.60 mmol, 6.00 eq.) and 4-indanol (CAS 1641-41-4, 26.8 mg, 0.20 mmol, 2.00 eq.). The resulting suspension was heated to 55° C. for 17 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as a light brown solid (51.0 mg).

LC-MS (Method 3): Rₜ=2.05 min, MS (ESIpos): m/z=622 [M+H]⁺.

¹H-NMR (300 MHz, CHLOROFORM-d) δ [ppm]: 7.58 (dd, 1H), 7.14 (m, 5H), 6.90 (m, 2H), 6.53 (d, 1H), 5.51 (d, 1H), 5.30 (d, 1H), 4.63 (d, 1H), 4.25 (m, 5H), 3.92 (m, 5H), 3.17 (dt, 1H), 3.04 (dt, 1H), 2.91 (q, 4H), 2.40 (m, 2H), 2.07 (m, 4H), 1.30 (t, 3H), 1.03 (t, 3H).

### Intermediate 83

### (rac)-Ethyl 1-(3-(4-chloro-3,5-dimethylphenoxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate

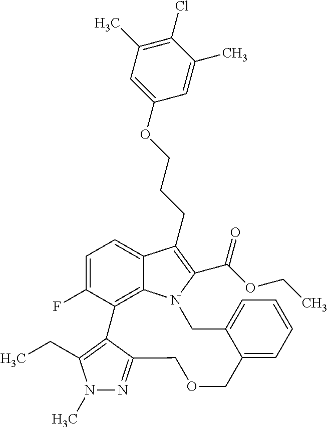 [SMILES: CCOC(=O)c1c(CCCOc2cc(C)c(Cl)c(C)c2)c2ccc(F)c3c2n1Cc1ccccc1COCc1nn(C)c(CC)c1-3]

To a stirred solution of (rac)-ethyl 1-(3-bromopropyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylate (see intermediate 76, 56.8 mg, 0.10 mmol, 1.00 eq.) in anhydrous tetrahydrofuran (1.00 mL, 0.10 M) were added cesium carbonate (195 mg, 0.60 mmol, 6.00 eq.) and 4-chloro-3,5-dimethylphenol (CAS 88-04-0, 31.3 mg, 0.20 mmol, 2.00 eq.). The resulting suspension was heated to 55° C. for 17 hours and cooled to room temperature. Celite was added to the solution, volatiles were removed under reduced pressure, and the residue was loaded onto a silica gel cartridge which was subjected to reverse phase column chromatography (10-100% acetonitrile/water with 0.1% formic acid gradient) to give the title compound as an off-white solid (55.0 mg).

LC-MS (Method 3): Rₜ=2.10 min, MS (ESIpos): m/z=644 [M+H]⁺.

¹H-NMR (300 MHz, CHLOROFORM-d) δ [ppm]: 7.56 (dd, 1H), 7.13 (d, 4H), 6.94 (t, 1H), 6.57 (s, 2H), 5.51 (d, 1H), 5.29 (d, 1H), 4.62 (d, 1H), 4.25 (m, 5H), 3.87 (m, 5H), 3.08 (m, 2H), 2.41 (dd, 1H), 2.36 (dd, 1H), 2.32 (s, 6H), 2.06 (m, 2H), 1.31 (t, 3H), 1.03 (t, 3H).

### Intermediate 84

### Ethyl 3-(3-((tert-butyldimethylsilyl)oxy)propyl)-6-fluoro-7-(5-(hydroxymethyl)-1,3-dimethyl-1H-pyrazol-4-yl)-1H-indole-2-carboxylate

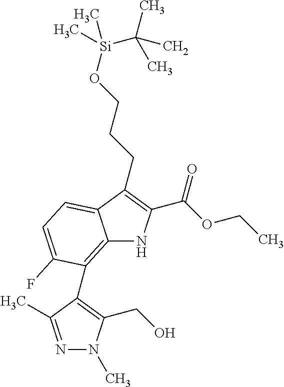 [SMILES: CCOC(=O)c1[nH]c2c(-c3c(C)nn(C)c3CO)c(F)ccc2c1CCCO[Si](C)(C)C(C)(C)C]

To a stirred suspension of (4-bromo-1,3-dimethyl-1H-pyrazol-5-yl)methanol (see intermediate 21, 3.89 g, 19.0 mmol, 1.00 eq.), XPhos Pd G3 (939 mg, 1.11 mmol, 7.00 mol %) and potassium phosphate tribasic (6.75 g, 31.8 mmol, 2.00 eq.) in a 2:1 mixture of 1,4-dioxane/water degassed with argon (47.7 mL) was slowly added dropwise (over a period of approximately 1 h) a solution of (rac)-ethyl 3-(3-((tert-butyldimethylsilyl)oxy)propyl)-6-fluoro-7-(4,4,5,5-tetramethyl-1,3,2-dioxa-borolan-2-yl)-1H-indole-2-carboxylate (see intermediate 62, 8.08 g, 15.9 mmol, 1.00 eq.) in 1,4-dioxane degassed with argon (15.9 mL, 1.00 M) at a temperature of 50° C. The resulting dark mixture was heated to 50° C. for further 30 minutes, cooled to room temperature and concentrated under reduced pressure. The residue was dissolved in water (50 mL) and extracted with ethyl acetate thrice (50 mL each). The combined organic extracts were washed with brine (50 mL), dried (magnesium sulfate) and filtered. Celite was added to the solution, volatiles were removed under reduced pressure, and the residue was loaded onto a silica gel cartridge which was subjected to flash column chromatography (0-100% acetone/dichloromethane gradient) and then reverse phase column chromatography (10-100% acetonitrile/water gradient) to give the title compound as a light yellow solid (4.14 g).

LC-MS (Method 3): Rₜ=1.94 min, MS (ESIpos): m/z=504 [M+H]⁺.

¹H-NMR (300 MHz, CHLOROFORM-d) δ [ppm]: ¹H NMR (Chloroform-d) δ: 9.00 (s, 1H), 7.65 (m, 1H), 6.99 (dd, 1H), 4.52 (m, 2H), 4.35 (m, 2H), 3.99 (s, 3H), 3.70 (t, 2H), 3.14 (m, 2H), 1.90 (m, 3H), 1.38 (t, 3H), 0.92 (s, 9H), 0.07 (s, 6H).

### Intermediate 85

### (rac)-Ethyl 1-(3-((tert-butyldimethylsilyl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate

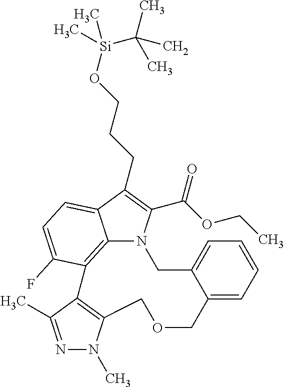 [SMILES: CCOC(=O)c1c(CCCO[Si](C)(C)C(C)(C)C)c2ccc(F)c3c2n1Cc1ccccc1COCc1c-3c(C)nn1C]

To a stirred solution of ethyl 3-(3-((tert-butyldimethylsilyl)oxy)propyl)-6-fluoro-7-(5-(hydroxymethyl)-1,3-dimethyl-1H-pyrazol-4-yl)-1H-indole-2-carboxylate (see intermediate 84, 2.04 g, 4.05 mmol, 1.00 eq.) in acetonitrile degassed with argon (40.4 mL, 0.10 M) was added cesium carbonate (6.58 g, 20.2 mmol, 5.00 eq.) in one portion at room temperature. After stirring for a further 10 min, 1,2-bis(bromomethyl)benzene (CAS 91-13-4, 1.17 g, 4.45 mmol, 1.10 eq.) and sodium iodide (1.21 g, 8.10 mmol, 2.00 eq.) were added and the resulting mixture was then heated to 40° C. for 17 hours. The mixture was cooled to room temperature and filtered through a Celite plug. Celite was added to the solution, volatiles were removed under reduced pressure, and the residue was loaded onto a silica gel cartridge which was subjected to flash column chromatography (0-100% ethyl acetate/hexanes gradient) to give the title compound as a white solid (1.88 g).

LC-MS (Method 3): Rₜ=6.01 min, MS (ESIpos): m/z=606 [M+H]⁺.

¹H-NMR (300 MHz, CHLOROFORM-d) δ [ppm]: 7.63 (dd, 1H), 7.26 (s, 2H), 7.11 (dtd, 2H), 6.92 (t, 1H), 6.76 (d, 1H), 5.46 (d, 1H), 5.34 (d, 1H), 4.60 (d, 1H), 4.24 (m, 5H), 3.94 (s, 3H), 3.66 (t, 2H), 3.13 (ddd, 1H), 3.01 (ddd, 1H), 1.90 (m, 5H), 1.33 (t, 3H), 0.92 (s, 9H), 0.05 (s, 6H).

### Intermediate 86

### (rac)-Ethyl 15-fluoro-1-(3-hydroxypropyl)-12,14-dimethyl-4,9,11,12-tetrahydrobenzo[3,4]pyrazolo[4′,3′:9,10][1]oxa[6]azacycloundecino[8,7,6-hi]indole-2-carboxylate

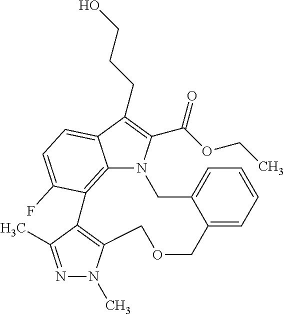 [SMILES: CCOC(=O)c1c(CCCO)c2ccc(F)c3c2n1Cc1ccccc1COCc1c-3c(C)nn1C]

To a stirred solution of (rac)-ethyl 1-(3-((tert-butyldimethylsilyl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 85, 1.88 g, 3.10 mmol, 1.00 eq.) in anhydrous tetrahydrofuran (62.0 mL, 0.05 M) was added a 1.0 M solution of tetrabutylammonium fluoride in tetrahydrofuran (3.72 mL, 3.72 mmol, 1.20 eq.) at a temperature of 0° C. The resulting light yellow mixture was warmed to room temperature, stirred for 2 hours, and then concentrated under reduced pressure. The residue was resuspended in saturated aqueous ammonium chloride solution (20 mL) and was then extracted with ethyl acetate thrice (20 mL each). The combined organic extracts were washed with brine (20 mL), dried (magnesium sulfate) and filtered. Celite was added to the solution, volatiles were removed under reduced pressure, and the residue was loaded onto a silica gel cartridge which was subjected to flash column chromatography (0-30% acetone/dichloromethane gradient) to give the title compound as a white solid (1.33 g).

LC-MS (Method 3): Rₜ=3.24 min, MS (ESIpos): m/z=492 [M+H]⁺.

¹H-NMR (300 MHz, CHLOROFORM-d) δ [ppm]: 7.60 (dd, 1H), 7.12 (dtd, 2H), 6.94 (t, 1H), 6.76 (dd, 1H), 5.37 (m, 2H), 4.61 (d, 1H), 4.24 (m, 5H), 3.94 (s, 3H), 3.59 (m, 2H), 3.14 (m, 2H), 2.41 (t, 1H), 1.94 (m, 5H), 1.33 (t, 3H).

### Intermediate 87

### (rac)-Ethyl 1-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate

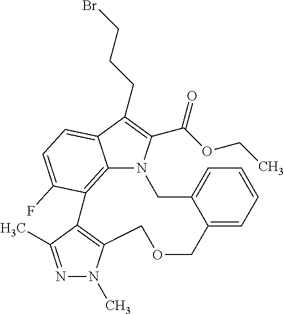 [SMILES: CCOC(=O)c1c(CCCBr)c2ccc(F)c3c2n1Cc1ccccc1COCc1c-3c(C)nn1C]

To a stirred solution of (rac)-ethyl 15-fluoro-1-(3-hydroxypropyl)-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 86, 1.32 g, 2.68 mmol, 1.00 eq.) and triphenylphosphane (771 mg, 2.94 mmol, 1.10 eq.) in anhydrous dichloromethane (26.8 mL, 0.10 M) was added carbon tetrabromide (974 mg, 2.94 mmol, 1.10 eq.) in one portion at a temperature of 0° C. The resulting mixture was warmed to room temperature and stirred for 1 hour. Celite was added to the mixture, volatiles were removed under reduced pressure, and the residue was subjected to flash column chromatography (0-15% acetone/dichloromethane gradient) to give the title compound as a white solid (1.25 g).

LC-MS (Method 3): Rₜ=4.58 min, MS (ESIpos): m/z=554 [M+H]⁺.

¹H-NMR (300 MHz, CHLOROFORM-d) δ [ppm]: 7.64 (dd, 1H), 7.27 (m, 3H), 7.16 (td, 1H), 7.08 (td, 1H), 6.96 (t, 1H), 6.76 (dd, 1H), 5.46 (d, 1H), 4.61 (d, 1H), 4.26 (m, 5H), 3.94 (s, 3H), 3.44 (m, 2H), 3.18 (m, 2H), 2.25 (m, 2H), 1.93 (s, 3H), 1.33 (t, 3H)

### Intermediate 88

### (rac)-Ethyl 15-fluoro-12,14-dimethyl-1-(3-(naphthalen-1-yloxy)propyl)-4,9,11,12-tetrahydrobenzo[3,4]pyrazolo[4′,3′:9,10][1]oxa[6]azacycloundecino[8,7,6-hi]indole-2-carboxylate

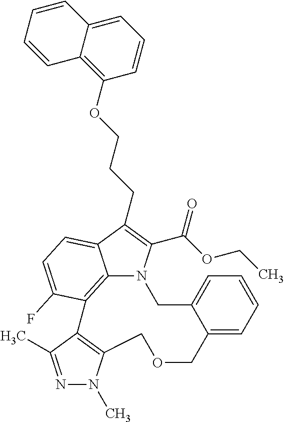 [SMILES: CCOC(=O)c1c(CCCOc2cccc3ccccc23)c2ccc(F)c3c2n1Cc1ccccc1COCc1c-3c(C)nn1C]

To a stirred solution of (rac)-ethyl 1-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 87, 170 mg, 0.30 mmol, 1.00 eq.) in anhydrous tetrahydrofuran (3.00 mL, 0.10 M) were added cesium carbonate (583 mg, 1.79 mmol, 6.00 eq.) and naphthalen-1-ol (CAS 90-15-3, 86.5 mg, 0.60 mmol, 2.00 eq.). The resulting suspension was heated to 55° C. for 20 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a brown solid (169 mg).

LC-MS (Method 3): Rₜ=5.40 min, MS (ESIpos): m/z=618 [M+H]⁺.

¹H-NMR (300 MHz, CHLOROFORM-d) δ [ppm]: 8.34 (m, 1H), 7.81 (m, 1H), 7.61 (dd, 1H), 7.49 (m, 2H), 7.42 (d, 1H), 7.34 (dd, 1H), 7.25 (m, 6H), 7.15 (td, 1H), 7.06 (td, 1H), 6.85 (t, 1H), 6.80 (d, 1H), 6.72 (dd, 1H), 5.46 (d, 1H), 5.37 (d, 1H), 4.60 (d, 1H), 4.19 (m, 7H), 3.94 (s, 3H), 3.31 (m, 2H), 2.32 (m, 2H), 1.94 (s, 3H), 1.28 (t, 3H).

### Intermediate 89

### (rac)-Ethyl 15-fluoro-1-(3-((6-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylate

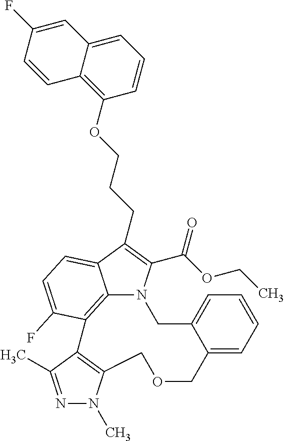 [SMILES: CCOC(=O)c1c(CCCOc2cccc3cc(F)ccc23)c2ccc(F)c3c2n1Cc1ccccc1COCc1c-3c(C)nn1C]

To a stirred solution of (rac)-ethyl 1-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 87, 170 mg, 0.30 mmol, 1.00 eq.) in anhydrous tetrahydrofuran (3.00 mL, 0.10 M) were added cesium carbonate (583 mg, 1.79 mmol, 6.00 eq.) and 6-fluoronaphthalen-1-ol (CAS 804498-72-4, 97.2 mg, 0.60 mmol, 2.00 eq.). The resulting suspension was heated to 55° C. for 20 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a brown solid (167.8 mg).

LC-MS (Method 3): Rₜ=5.46 min, MS (ESIpos): m/z=638 [M+H]⁺.

¹H-NMR (300 MHz, CHLOROFORM-d) δ [ppm]: 8.32 (dd, 1H), 7.59 (dd, 1H), 7.37 (m, 3H), 7.22 (m, 7H), 7.06 (m, 1H), 6.86 (t, 1H), 6.79 (d, 1H), 6.66 (dd, 1H), 5.39 (m, 2H), 4.60 (d, 1H), 4.18 (m, 7H), 3.93 (d, 3H), 3.32 (m, 2H), 2.32 (m, 2H), 1.93 (s, 3H), 1.27 (m, 3H).

### Intermediate 90

### (rac)-Ethyl 15-fluoro-1-(3-((4-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylate

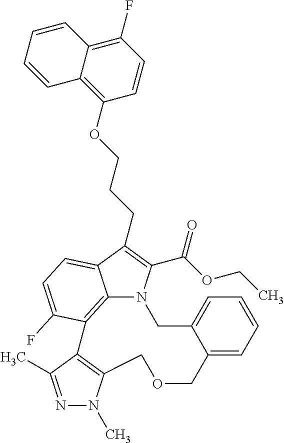 [SMILES: CCOC(=O)c1c(CCCOc2ccc(F)c3ccccc23)c2ccc(F)c3c2n1Cc1ccccc1COCc1c-3c(C)nn1C]

To a stirred solution of (rac)-ethyl-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 87, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 4-fluoronaphthalen-1-ol (CAS 315-53-7, 64.8 mg, 0.40 mmol, 2.00 eq.). The resulting suspension was heated to 55° C. for 20 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a brown solid (112.7 mg).

LC-MS (Method 3): Rₜ=5.51 min, MS (ESIpos): m/z=636 [M+H]⁺.

¹H-NMR (300 MHz, CHLOROFORM-d) δ [ppm]: 8.31 (m, 1H), 8.05 (m, 1H), 7.57 (m, 3H), 7.25 (m, 6H), 7.15 (td, 1H), 7.03 (m, 2H), 6.85 (t, 1H), 6.79 (d, 1H), 6.57 (dd, 1H), 5.45 (d, 1H), 5.37 (d, 1H), 4.60 (d, 1H), 4.18 (m, 7H), 3.94 (s, 3H), 3.31 (m, 2H), 2.32 (m, 2H), 1.94 (s, 3H), 1.28 (t, 3H).

### Intermediate 91

### (rac)-Ethyl 15-fluoro-12,14-dimethyl-1-(3-((5,6,7,8-tetrahydronaphthalen-1-yl)oxy)propyl)-4,9,11,12-tetrahydrobenzo[3,4]pyrazolo[4′,3′:9,10][1]oxa[6]azacycloundecino[8,7,6-hi]indole-2-carboxylate

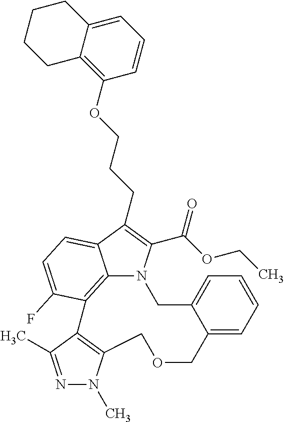 [SMILES: CCOC(=O)c1c(CCCOc2cccc3c2CCCC3)c2ccc(F)c3c2n1Cc1ccccc1COCc1c-3c(C)nn1C]

To a stirred solution of (rac)-ethyl 1-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 87, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 5,6,7,8-tetrahydronaphthalen-1-ol (CAS 529-35-1, 59.2 mg, 0.40 mmol, 2.00 eq.). The resulting suspension was heated to 55° C. for 20 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a brown solid (108 mg).

LC-MS (Method 3): Rₜ=5.80 min, MS (ESIpos): m/z=622 [M+H]⁺.

¹H-NMR (300 MHz, CHLOROFORM-d) δ [ppm]: 7.60 (dd, 1H), 7.26 (d, 4H), 7.15 (td, 1H), 7.06 (m, 2H), 6.91 (t, 1H), 6.78 (d, 1H), 6.70 (d, 1H), 6.57 (d, 1H), 5.46 (d, 1H), 5.36 (d, 1H), 4.61 (d, 1H), 4.16 (m, 10H), 3.22 (m, 2H), 2.75 (m, 4H), 2.16 (m, 2H), 1.94 (s, 3H), 1.79 (d, 4H), 1.31 (t, 3H).

### Intermediate 92

### (rac)-Ethyl 1-(3-((2,3-dihydro-1H-inden-4-yl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate

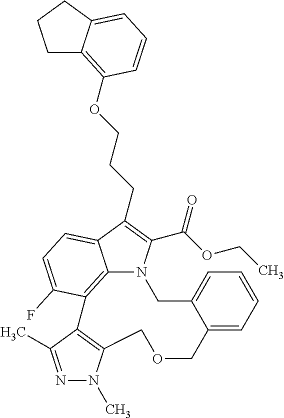 [SMILES: CCOC(=O)c1c(CCCOc2cccc3c2CCC3)c2ccc(F)c3c2n1Cc1ccccc1COCc1c-3c(C)nn1C]

To a stirred solution of (rac)-ethyl 1-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 87, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 4-indanol (CAS 1641-41-4, 53.6 mg, 0.40 mmol, 2.00 eq.). The resulting suspension was heated to 55° C. for 20 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a brown solid (97.5 mg).

LC-MS (Method 3): Rₜ=5.52 min, MS (ESIpos): m/z=608 [M+H]⁺.

¹H-NMR (300 MHz, CHLOROFORM-d) δ [ppm]: 7.60 (dd, 1H), 7.26 (dd, 4H), 7.15 (td, 1H), 7.07 (td, 2H), 6.91 (t, 1H), 6.85 (d, 1H), 6.79 (m, 1H), 6.58 (d, 1H), 5.46 (d, 1H), 5.36 (d, 1H), 4.61 (d, 1H), 4.16 (m, 8H), 3.94 (s, 3H), 3.20 (m, 2H), 2.92 (m, 4H), 2.11 (m, 4H), 1.94 (s, 3H), 1.31 (t, 3H).

### Intermediate 93

### (rac)-Ethyl 1-(3-(4-chloro-3,5-dimethylphenoxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate

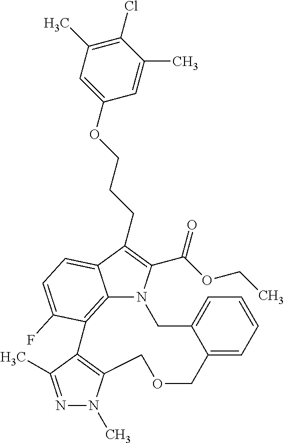 [SMILES: CCOC(=O)c1c(CCCOc2cc(C)c(Cl)c(C)c2)c2ccc(F)c3c2n1Cc1ccccc1COCc1c-3c(C)nn1C]

To a stirred solution of (rac)-ethyl 1-(3-bromopropyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 87, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 4-chloro-3,5-dimethylphenol (CAS 88-04-0, 62.6 mg, 0.40 mmol, 2.00 eq.). The resulting suspension was heated to 55° C. for 20 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a brown solid (98.9 mg).

LC-MS (Method 3): Rₜ=5.69 min, MS (ESIpos): m/z=630 [M+H]⁺.

¹H-NMR (300 MHz, CHLOROFORM-d) δ [ppm]: ¹H NMR (Chloroform-d) δ: 7.59 (dd, 1H), 7.26 (dd, 4H), 7.15 (td, 1H), 7.05 (td, 1H), 6.90 (t, 1H), 6.77 (d, 1H), 6.59 (m, 2H), 5.46 (d, 1H), 5.36 (d, 1H), 4.61 (d, 1H), 4.23 (m, 5H), 3.93 (m, 5H), 3.19 (m, 2H), 2.32 (d, 6H), 2.13 (q, 2H), 1.93 (s, 3H), 1.32 (t, 3H).

### Intermediate 94

### Ethyl 3-(3-((tert-butyldimethylsilyl)oxy)propyl)-7-(3-ethyl-5-(hydroxymethyl)-1-methyl-1H-pyrazol-4-yl)-6-fluoro-1H-indole-2-carboxylate

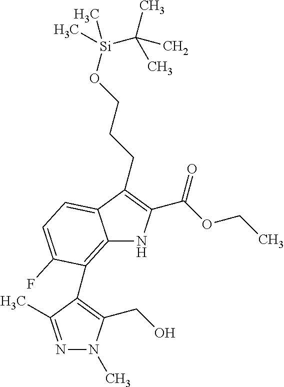 [SMILES: CCOC(=O)c1[nH]c2c(-c3c(C)nn(C)c3CO)c(F)ccc2c1CCCO[Si](C)(C)C(C)(C)C]

To a stirred suspension of (4-bromo-3-ethyl-1-methyl-1H-pyrazol-5-yl)methanol (see intermediate 19, 3.81 g, 17.4 mmol, 1.00 eq.), XPhos Pd G3 (854 mg, 1.01 mmol, 7.00 mol %) and potassium phosphate tribasic (6.15 g, 29.0 mmol, 2.00 eq.) in a 2:1 mixture of 1,4-dioxane/water degassed with argon (43.5 mL) was slowly added dropwise (over a period of approximately 1 h) a solution of ethyl 3-(3-((tert-butyldimethylsilyl)oxy)propyl)-6-fluoro-7-(4,4,5,5-tetramethyl-1,3,2-dioxaborolan-2-yl)-1H-indole-2-carboxylate (see intermediate 62, 7.38 g, 14.5 mmol, 1.00 eq.) in 1,4-dioxane degassed with argon (14.5 mL, 1.00 M) at a temperature of 50° C. The resulting dark mixture was heated to 50° C. for further 30 minutes, cooled to room temperature and concentrated under reduced pressure. The residue was dissolved in water (50 mL) and extracted with ethyl acetate thrice (50 mL each). The combined organic extracts were washed with brine (50 mL), dried (magnesium sulfate) and filtered. Celite was added to the solution, volatiles were removed under reduced pressure, and the residue was subjected to flash column chromatography (0-100% acetone/dichloromethane gradient) and then reverse phase column chromatography (10-100% acetonitrile/water gradient) to give the title compound as a yellow solid (4.79 g).

LC-MS (Method 3): Rₜ=2.01 min, MS (ESIpos): m/z=518 [M+H]⁺.

¹H-NMR (300 MHz, CHLOROFORM-d) δ [ppm]: 8.88 (s, 1H), 7.65 (m, 1H), 6.99 (dd, 1H), 4.50 (q, 2H), 4.36 (q, 2H), 4.00 (s, 3H), 3.70 (t, 2H), 3.14 (m, 2H), 2.51 (qd, 2H), 1.91 (m, 3H), 1.38 (t, 3H), 1.11 (t, 3H), 0.92 (s, 9H), 0.07 (s, 6H).

### Intermediate 95

### (rac)-Ethyl 1-(3-((tert-butyldimethylsilyl)oxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate

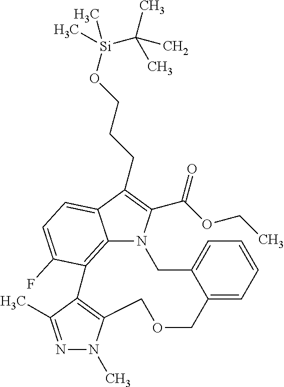 [SMILES: CCOC(=O)c1c(CCCO[Si](C)(C)C(C)(C)C)c2ccc(F)c3c2n1Cc1ccccc1COCc1c-3c(C)nn1C]

To a stirred solution of ethyl 3-(3-((tert-butyldimethylsilyl)oxy)propyl)-7-(3-ethyl-5-(hydroxymethyl)-1-methyl-1H-pyrazol-4-yl)-6-fluoro-1H-indole-2-carboxylate (see intermediate 94, 3.59 g, 7.12 mmol, 1.00 eq.) in acetonitrile (47.3 mL, 0.10 M) was added cesium carbonate (7.68 g, 23.6 mmol, 5.00 eq.) in one portion. After stirring for a further 10 min, 1,2-bis(bromomethyl)benzene (CAS 91-13-4, 1.37 g, 5.20 mmol, 1.10 eq.) and sodium iodide (1.41 g, 9.46 mmol, 2.00 eq.) was added and the resulting mixture was then heated to 40° C. for 19 hours. The mixture was cooled to room temperature and filtered through a Celite plug. Celite was added to the solution, volatiles were removed under reduced pressure, and the residue was subjected to flash column chromatography (0-100% ethyl acetate/hexanes gradient) to give the title compound as a white solid (1.93 g).

LC-MS (Method 3): Rₜ=6.20 min, MS (ESIpos): m/z=620 [M+H]⁺.

¹H-NMR (300 MHz, CHLOROFORM-d) δ [ppm]: 7.63 (dd, 1H), 7.24 (d, 1H), 7.15 (td, 1H), 7.07 (td, 1H), 6.80 (m, 1H), 5.44 (d, 1H), 5.35 (d, 1H), 4.61 (d, 1H), 4.29 (tp, 2H), 4.19 (d, 2H), 4.10 (d, 1H), 3.95 (s, 3H), 3.65 (t, 2H), 3.12 (ddd, 1H), 2.99 (ddd, 1H), 2.29 (qd, 2H), 1.87 (m, 2H), 1.32 (t, 3H), 1.00 (t, 3H), 0.92 (s, 9H), 0.05 (s, 6H).

### Intermediate 96

### (rac)-Ethyl 14-ethyl-15-fluoro-1-(3-hydroxypropyl)-12-methyl-4,9,11,12-tetrahydrobenzo[3,4]pyrazolo[4′,3′:9,10][1,6]oxaazacycloundecino[8,7,6-hi]indole-2-carboxylate

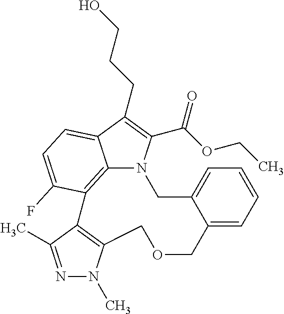 [SMILES: CCOC(=O)c1c(CCCO)c2ccc(F)c3c2n1Cc1ccccc1COCc1c-3c(C)nn1C]

To a stirred solution of (rac)-ethyl 1-(3-((tert-butyldimethylsilyl)oxy)propyl)-14-ethyl-15-fluoro-12-methyl-4,9,11,12-tetrahydrobenzo[3,4]pyrazolo[4′,3′:9,10][1,6]oxaazacycloundecino-[8,7,6-hi]indole-2-carboxylate (see intermediate 95, 1.92 g, 3.09 mmol, 1.00 eq.) in anhydrous tetrahydrofuran (61.7 mL, 0.05 M) was added a 1.0 M solution of tetrabutylammonium fluoride in tetrahydrofuran (3.70 mL, 3.70 mmol, 1.20 eq.) at a temperature of 0° C. The resulting light yellow mixture was warmed to room temperature, stirred for 2 hours, and was then concentrated under reduced pressure. The residue was resuspended in saturated aqueous ammonium chloride solution (20 mL) and extracted with ethyl acetate thrice (20 mL each). The combined organic extracts were washed with brine (20 mL), dried (magnesium sulfate) and filtered. Celite was added to the solution, volatiles were removed under reduced pressure, and the residue was subjected to flash column chromatography (0-30% acetone/dichloromethane gradient) to give the title compound as a white solid (1.42 g).

LC-MS (Method 3): Rₜ=3.43 min, MS (ESIpos): m/z=506 [M+H]⁺.

¹H-NMR (300 MHz, CHLOROFORM-d) δ [ppm]: 7.60 (dd, 1H), 7.27 (m, 3H), 7.13 (dtd, 2H), 6.94 (t, 1H), 6.80 (dd, 1H), 5.40 (d, 1H), 5.32 (d, 1H), 4.61 (d, 1H), 4.33 (q, 2H), 4.19 (s, 2H), 4.10 (d, 1H), 3.95 (s, 3H), 3.56 (s, 2H), 3.13 (m, 2H), 2.31 (m, 3H), 1.96 (m, 2H), 1.33 (t, 3H), 1.01 (t, 3H).

### Intermediate 97

### (rac)-Ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate

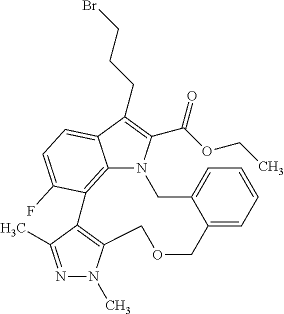 [SMILES: CCOC(=O)c1c(CCCBr)c2ccc(F)c3c2n1Cc1ccccc1COCc1c-3c(C)nn1C]

To a stirred solution of (rac)-ethyl 14-ethyl-15-fluoro-1-(3-hydroxypropyl)-12-methyl-4,9,11,12-tetrahydrobenzo[3,4]pyrazolo[4′,3′:9,10][1,6]oxaazacycloundecino[8,7,6-hi]indole-2-carboxylate (see intermediate 96, 1.40 g, 2.76 mmol, 1.00 eq.) and triphenylphosphane (794 mg, 3.03 mmol, 1.10 eq.) in anhydrous dichloromethane (27.5 mL, 0.10 M) was added carbon tetrabromide (1.00 g, 3.03 mmol, 1.10 eq.) in one portion at a temperature of 0° C. The resulting mixture was warmed to room temperature and stirred for 1 hour. Celite was added to the mixture, volatiles were removed under reduced pressure, and the residue was subjected to flash column chromatography (0-15% acetone/dichloromethane gradient) to give the title compound as a fluffy white solid (1.35 g).

LC-MS (Method 3): Rₜ=4.77 min, MS (ESIpos): m/z=568 [M+H]⁺.

¹H-NMR (300 MHz, CHLOROFORM-d) δ [ppm]: 7.64 (dd, 1H), 7.26 (dd, 2H), 7.16 (td, 1H), 7.08 (td, 1H), 6.95 (t, 1H), 6.80 (dd, 1H), 5.40 (m, 2H), 4.61 (d, 1H), 4.34 (dd, 1H), 4.29 (dd, 1H), 4.19 (s, 2H), 4.10 (d, 1H), 3.95 (s, 3H), 3.44 (m, 2H), 3.17 (m, 2H), 2.28 (m, 4H), 1.33 (t, 3H), 1.00 (t, 3H).

### Intermediate 98

### (rac)-Ethyl 1-(3-(4-chloro-3,5-dimethylphenoxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate

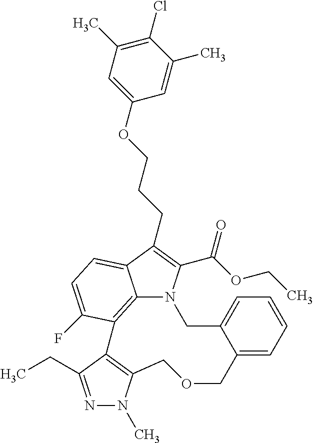 [SMILES: CCOC(=O)c1c(CCCOc2cc(C)c(Cl)c(C)c2)c2ccc(F)c3c2n1Cc1ccccc1COCc1c-3c(CC)nn1C]

To a stirred solution of (rac)-ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate (see intermediate 97, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 4-chloro-3,5-dimethylphenol (CAS 88-04-0, 62.6 mg, 0.40 mmol, 2.00 eq.). The resulting suspension was heated to 55° C. for 21 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as an off-white solid (108 mg).

LC-MS (Method 3): Rₜ=5.90 min, MS (ESIpos): m/z=644 [M+H]⁺.

¹H-NMR (300 MHz, CHLOROFORM-d) δ [ppm]: 7.59 (dd, 1H), 7.25 (d, 2H), 7.10 (dtd, 2H), 6.86 (m, 2H), 6.59 (s, 2H), 5.44 (d, 1H), 5.37 (d, 1H), 4.61 (d, 1H), 4.31 (dd, 1H), 4.26 (dd, 1H), 4.19 (m, 2H), 4.10 (d, 1H), 3.94 (m, 5H), 3.18 (m, 2H), 2.27 (m, 8H), 2.15 (m, 2H), 1.32 (t, 3H), 1.00 (t, 3H).

### Intermediate 99

### (rac)-Ethyl 1-(3-((2,3-dihydro-1H-inden-4-yl)oxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate

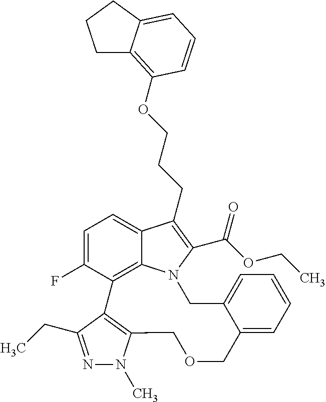 [SMILES: CCOC(=O)c1c(CCCOc2cccc3c2CCC3)c2ccc(F)c3c2n1Cc1ccccc1COCc1c-3c(CC)nn1C]

To a stirred solution of (rac)-ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate (see intermediate 97, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 4-indanol (CAS 1641-41-4, 53.6 mg, 0.40 mmol, 2.00 eq.). The resulting suspension was heated to 55° C. for 21 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as an off-white solid (102 mg).

LC-MS (Method 3): Rₜ=5.73 min, MS (ESIpos): m/z=622 [M+H]⁺.

¹H-NMR (300 MHz, CHLOROFORM-d) δ [ppm]: 7.53 (dd, 1H), 7.18 (d, 2H), 7.04 (m, 3H), 6.80 (dt, 3H), 6.50 (d, 1H), 5.37 (d, 1H), 5.30 (d, 1H), 4.54 (d, 1H), 4.21 (m, 2H), 4.12 (s, 2H), 4.03 (d, 1H), 3.94 (m, 2H), 3.88 (s, 3H), 3.11 (m, 2H), 2.85 (m, 4H), 2.23 (m, 2H), 2.04 (m, 4H), 1.23 (t, 3H), 0.93 (t, 3H).

### Intermediate 100

### (rac)-Ethyl 14-ethyl-15-fluoro-12-methyl-1-(3-((5,6,7,8-tetrahydronaphthalen-1-yl)oxy)propyl)-4,9,11,12-tetrahydrobenzo[3,4]pyrazolo[4′,3′:9,10][1,6]oxaazacycloundecino[8,7,6-hi]indole-2-carboxylate

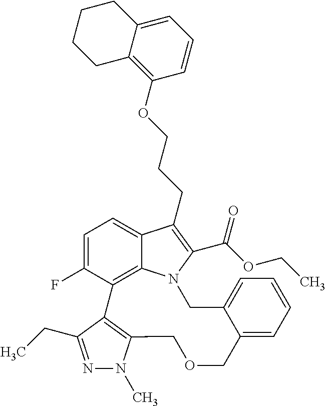 [SMILES: CCOC(=O)c1c(CCCOc2cccc3c2CCCC3)c2ccc(F)c3c2n1Cc1ccccc1COCc1c-3c(CC)nn1C]

To a stirred solution of (rac)-ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate (see intermediate 97, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 5,6,7,8-tetrahydronaphthalen-1-ol (CAS 529-35-1, 59.2 mg, 0.40 mmol, 2.00 eq.). The resulting suspension was heated to 55° C. for 21 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as an off-white solid (111 mg).

LC-MS (Method 3): Rₜ=6.00 min, MS (ESIpos): m/z=636 [M+H]⁺.

¹H-NMR (300 MHz, CHLOROFORM-d) δ [ppm]: 7.60 (dd, 1H), 7.25 (m, 4H), 7.10 (m, 3H), 6.91 (t, 1H), 6.82 (m, 1H), 6.69 (dd, 1H), 6.56 (d, 1H), 5.44 (d, 1H), 5.37 (d, 1H), 4.61 (d, 1H), 4.28 (m, 2H), 4.19 (s, 2H), 4.10 (d, 1H), 3.96 (m, 5H), 3.20 (m, 2H), 2.75 (m, 4H), 2.25 (m, 4H), 1.79 (m, 4H), 1.30 (t, 3H), 1.00 (t, 3H).

### Intermediate 101

### (rac)-Ethyl 14-ethyl-15-fluoro-1-(3-((4-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylate

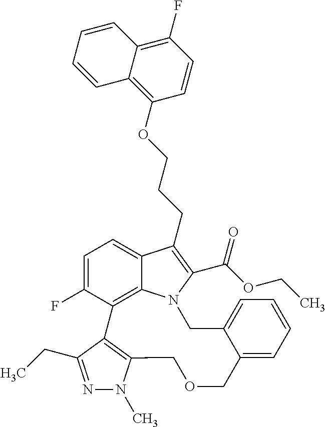 [SMILES: CCOC(=O)c1c(CCCOc2ccc(F)c3ccccc23)c2ccc(F)c3c2n1Cc1ccccc1COCc1c-3c(CC)nn1C]

To a stirred solution of (rac)-ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate (see intermediate 97, 113 mg, 0.20 mmol, 1.00 eq.) in anhydrous tetrahydrofuran (2.00 mL, 0.10 M) were added cesium carbonate (390 mg, 1.20 mmol, 6.00 eq.) and 4-fluoronaphthalen-1-ol (CAS 315-53-7, 64.8 mg, 0.40 mmol, 2.00 eq.). The resulting suspension was heated to 55° C. for 21 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a tan solid (118 mg).

LC-MS (Method 3): Rₜ=5.69 min, MS (ESIpos): m/z=650 [M+H]⁺.

¹H-NMR (300 MHz, CHLOROFORM-d) δ [ppm]: 8.31 (m, 1H), 8.05 (m, 1H), 7.57 (m, 3H), 7.24 (m, 2H), 7.08 (m, 3H), 6.84 (m, 2H), 6.56 (dd, 1H), 5.41 (m, 2H), 4.60 (d, 1H), 4.17 (m, 7H), 3.95 (s, 3H), 3.30 (m, 2H), 2.30 (m, 4H), 1.27 (t, 3H), 1.00 (t, 3H).

### Intermediate 102

### (rac)-Ethyl 14-ethyl-15-fluoro-1-(3-((6-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylate

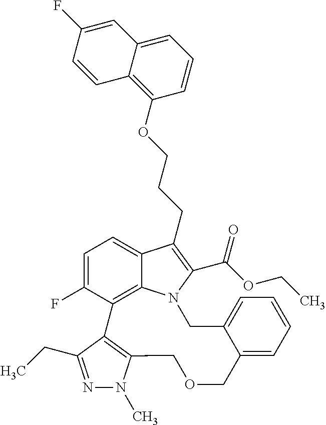 [SMILES: CCOC(=O)c1c(CCCOc2cccc3cc(F)ccc23)c2ccc(F)c3c2n1Cc1ccccc1COCc1c-3c(CC)nn1C]

To a stirred solution of (rac)-ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate (see intermediate 97, 170 mg, 0.30 mmol, 1.00 eq.) in anhydrous tetrahydrofuran (3.00 mL, 0.10 M) were added cesium carbonate (583 mg, 1.79 mmol, 6.00 eq.) and 6-fluoronaphthalen-1-ol (CAS 804498-72-4, 97.2 mg, 0.60 mmol, 2.00 eq.). The resulting suspension was heated to 55° C. for 21 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a tan solid (185 mg).

LC-MS (Method 3): Rₜ=5.64 min, MS (ESIpos): m/z=650 [M+H]⁺.

¹H-NMR (300 MHz, CHLOROFORM-d) δ [ppm]: 8.25 (dd, 1H), 7.52 (dd, 1H), 7.34 (dd, 1H), 7.28 (m, 2H), 7.12 (m, 5H), 6.77 (m, 2H), 6.58 (dd, 1H), 5.34 (m, 2H), 4.53 (d, 1H), 4.11 (m, 7H), 3.88 (s, 3H), 3.22 (m, 2H), 2.23 (m, 4H), 1.20 (t, 3H), 0.93 (t, 3H).

### Intermediate 103

### (rac)-Ethyl 14-ethyl-15-fluoro-12-methyl-1-(3-(naphthalen-1-yloxy)propyl)-4,9,11,12-tetrahydrobenzo[3,4]pyrazolo[4′,3′:9,10][1,6]oxaazacycloundecino[8,7,6-hi]indole-2-carboxylate

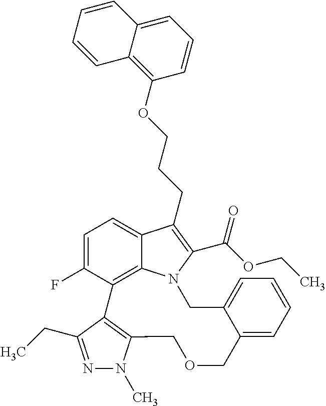 [SMILES: CCOC(=O)c1c(CCCOc2cccc3ccccc23)c2ccc(F)c3c2n1Cc1ccccc1COCc1c-3c(CC)nn1C]

To a stirred solution of (rac)-ethyl 1-(3-bromopropyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylate (see intermediate 97, 170 mg, 0.30 mmol, 1.00 eq.) in anhydrous tetrahydrofuran (3.00 mL, 0.10 M) were added cesium carbonate (583 mg, 1.79 mmol, 6.00 eq.) and naphthalen-1-ol (CAS 90-15-3, 86.5 mg, 0.60 mmol, 2.00 eq.). The resulting suspension was heated to 55° C. for 21 hours and cooled to room temperature. Celite was added to the suspension, volatiles were removed under reduced pressure, and the residue was subjected to reverse phase column chromatography (10-100% acetonitrile/water with 0.1% formic acid gradient) to give the title compound as a tan solid (173 mg).

LC-MS (Method 3): Rₜ=5.60 min, MS (ESIpos): m/z=632 [M+H]⁺.

¹H-NMR (300 MHz, CHLOROFORM-d) δ [ppm]: 8.36 (m, 1H), 7.82 (m, 1H), 7.63 (dd, 1H), 7.43 (m, 4H), 7.25 (m, 2H), 7.17 (td, 1H), 7.08 (td, 1H), 6.86 (m, 2H), 6.72 (dd, 1H), 5.42 (m, 2H), 4.61 (d, 1H), 4.20 (m, 7H), 3.96 (s, 3H), 3.33 (m, 2H), 2.32 (m, 4H), 1.28 (t, 3H), 1.02 (t, 3H).

### Intermediate 104

### tert-butoxyacetaldehyde

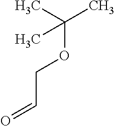

Sulfur trioxide pyridine complex (40.4 g, 254 mmol) in 250 mL of dimethyl sulfoxide was added to 2-tert-butoxyethanol (CAS 7580-85-0, 11 mL, 85 mmol) and triethylamine (35 mL, 250 mmol) dissolved in 240 mL of dichloromethane at 0° C. over 45 minutes. The reaction mixture was then warmed to room temperature and stirred overnight under argon. The reaction mixture was diluted with diethyl ether and washed with 10% aqueous citric acid followed by brine. The organic phase was dried over magnesium sulfate, passed through a plug of silica and the solvent was removed under reduced pressure to give 8.47 g of the title compound, which was taken to the next step without further purification.

### Intermediate 105

### N-(2-tert-butoxyethylidene)hydroxylamine

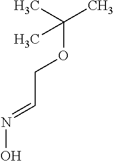

Hydroxylamine hydrochloride (6.08 g, 87.5 mmol) in a solution of aqueous sodium hydroxide (44 mL, 2.0 M, 87 mmol) was added to tert-butoxyacetaldehyde (see Intermediate 104, 8.47 g, 72.9 mmol) dissolved in 400 mL of ethanol and stirred overnight at 90° C. The reaction was concentrated, dissolved in ethyl acetate and the organic phase was washed with brine. The organic layer was dried over magnesium sulfate, the solvent was removed under reduced pressure and the residue was purified by normal phase chromatography (Biotage isolera, 30 g KP-Sil sphere cartridge) with 0-30% ethyl acetate in heptanes as eluent to afford 3.36 g of the title compound.

¹H NMR (400 MHz, d6-DMSO) δ [ppm]=1.12 (s, 9H), 3.88 (d, 1H), 4.10 (d, 1H), 6.66 (t, 0.5H), 7.24 (t, 0.5H), 10.71 (s, 0.5H), 11.00 (s, 0.5H)

### Intermediate 106

### 2-tert-butoxy-N-hydroxyethanimidoyl Chloride

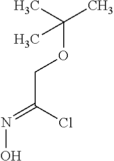

1-Chloropyrrolidine-2,5-dione (3.41 g, 25.5 mmol) was added to N-(2-tert-butoxyethylidene)hydroxylamine (see Intermediate 105, 3.35 g, 25.5 mmol) dissolved in N,N-dimethylformamide and stirred at room temperature for 6 hours. The reaction mixture was poured into diethyl ether and washed with brine. The organic layer was dried over magnesium sulfate, filtered and the solvent was removed under reduced pressure to give 4.10 g of the title compound, which was used without further purification.

¹H NMR (400 MHz, CDCl3) δ [ppm]=1.25 (s, 9H), 4.17 (s, 2H).

### Intermediate 107

### ethyl-3-(pyrrolidin-1-yl)prop-2-enoate

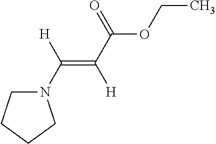

Ethyl prop-2-ynoate (CAS 623-47-2, 21 mL, 200 mmol) and pyrrolidine (CAS 123-75-1, 17 mL, 200 mmol) were dissolved in 1.0 L of acetonitrile and stirred overnight at room temperature. The solvent was removed under reduced pressure and the residue was recrystalised twice from heptane to afford 9.47 g of the desired compound.

¹H NMR (400 MHz, CDCl-3): δ [ppm]=1.24 (t, 3H), 1.91 (s, 4H), 2.97-3.60 (m, 4H), 4.11 (q, 2H), 4.46 (d, 1H), 7.63 (d, 1H).

### Intermediate 108

### ethyl 3-(tert-butoxymethyl)-1,2-oxazole-4-carboxylate

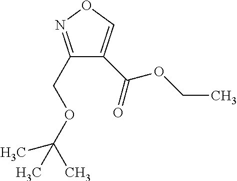 [SMILES: CCOC(=O)c1conc1COC(C)(C)C]

Ethyl-3-(pyrrolidin-1-yl)prop-2-enoate (see Intermediate 107, 4.19 g, 24.8 mmol) and 2-tert-butoxy-N-hydroxyethanimidoyl chloride (see Intermediate 106, 4.10 g, 24.8 mmol) were dissolved in 60 mL of tetrahydrofuran and stirred at reflux overnight. The reaction was quenched with water, neutralised with saturated aqueous sodium bicarbonate solution and acidified with aqueous 2M hydrochloric acid. The mixture was extracted with ethyl acetate, the phases were separated and the organic was washed with 2M aqueous hydrochloric acid, saturated aqueous sodium bicarbonate and brine. The organic phase was dried over magnesium sulfate, filtered through a 1 cm plug of silica and the solvent removed under reduced pressure to give 5.04 g of the desired compound.

¹H NMR (400 MHz, CDCl3) δ [ppm]=1.30 (s, 9H), 1.35 (t, 3H), 4.32 (q, 2H), 4.75 (s, 2H), 8.84 (s, 1H).

UPLC1-MS (Long acidic): Rt=1.95 min., MS (ESIpos): m/z=(fragment)+126.0

### Intermediate 109

### ethyl 3-(hydroxymethyl)-1,2-oxazole-4-carboxylate

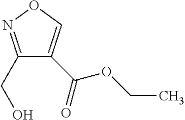

Trifluoroacetic acid (41 mL, 530 mmol) was added to ethyl 3-(tert-butoxymethyl)-1,2-oxazole-4-carboxylate (see Intermediate 108, 2.00 g, 8.80 mmol) dissolved in 40 mL of dichloromethane and stirred for 4 hours at room temperature. The mixture was washed with water and saturated aqueous sodium bicarbonate solution, followed by brine and the organic phase was dried over sodium sulfate, filtered and the solvent was removed under reduced pressure to afford 1.37 g, which appears to be a mixture of alcohol and trifluoroacetic acid ester in a 66:34 ratio by 1H NMR. It was taken on to the next step without further purification.

### Intermediate 110

### (1,2-oxazole-3,4-diyl)dimethanol

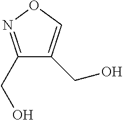

To a mixture of ethyl 3-{[(trifluoroacetyl)oxy]methyl}-1,2-oxazole-4-carboxylate and-ethyl 3-(hydroxymethyl)-1,2-oxazole-4-carboxylate (34:66) (see Intermediate 109, 1.37 g, 6.02 mmol) in 30 mL of tetrahydrofuran at 0° C. was added lithium aluminum hydride (457 mg, 12.0 mmol). After 1 hour of stirring at 0° C. a solution of aqueous sodium hydroxide (1.0 mL, 2.0 M, 2.0 mmol) and then solid sodium sulfate were added. The mixture was stirred for 30 minutes, filtered and washed with ethyl acetate. The filtrate was concentrated under vacuo to give 780 mg of the title compound. The material was taken through to the next step without purification.

### Intermediate 111

### 4-bromo-3-(bromomethyl)-1,5-dimethyl-1H-pyrazole

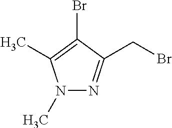

Phosphorous tribromide (CAS 7789-60-8, 12 mL, 120 mmol) was added to (4-bromo-1,5-dimethyl-1H-pyrazol-3-yl)methanol (see intermediate 12, 25.0 g, 122 mmol) dissolved in 230 mL of dichloromethane and stirred at room temperature for 4 hours. The reaction was quenched through addition to saturated aqueous sodium bicarbonate solution. The phases were separated and the organic was washed with brine. The organic phase was dried over magnesium sulfate, filtered through a plug of silica, which was washed with ethyl acetate, and the solvent was removed under reduced pressure to give 26.8 g of the desired compound without further purification.

¹H NMR (400 MHz, CDCl3) δ [ppm]=2.23 (s, 3H), 3.77 (s, 3H), 4.42 (s, 2H).

UPLC1-MS (Long acidic): Rt=1.81 min., MS (ESIpos): m/z=(M+H)+267/269/271.

### Intermediate 112

### (3-{[(4-bromo-1,5-dimethyl-1H-pyrazol-3-yl)methoxy]methyl}-1,2-oxazol-4-yl)methanol

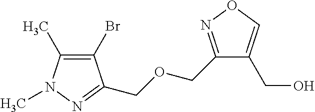 [SMILES: Cc1c(Br)c(COCc2nocc2CO)nn1C]

To a stirred suspension of sodium hydride (266 mg, 60% purity, 6.65 mmol) in 1 mL of tetrahydrofuran was added 1,2-oxazole-3,4-diyl)dimethanol (see Intermediate 110, 780 mg, 6.04 mmol) as a solution in 2 mL of tetrahydrofuran. After 10 minutes 4-bromo-3-(bromomethyl)-1,5-dimethyl-1H-pyrazole (see Intermediate 111, 1.62 g, 6.04 mmol) was added as a solution in tetrahydrofuran. The mixture was stirred at 65° C. for 6 hours under argon. It was quenched with water. The organic phase was dried and concentrated under reduced pressure. The crude material was purified by flash chromatography (column 30 g, 0-100% ethyl acetate/heptane) to give 210 mg as a mixture of regio-isomers of the desired product and (4-{[(4-bromo-1,5-dimethyl-1H-pyrazol-3-yl)methoxy]methyl}-1,2-oxazol-3-yl)methanol. H-NMR shows approximate 7:1 by the CH on the isoxazole. It was taken forward to the next step without further purification.

### Intermediate 113

### ethyl 6-chloro-7-[3-({[4-(hydroxymethyl)-1,2-oxazol-3-yl]methoxy}methyl)-1,5-dimethyl-1H-pyrazol-4-yl]-3-{3-[(naphthalen-1-yl)oxy]propyl}-1H-indole-2-carboxylate

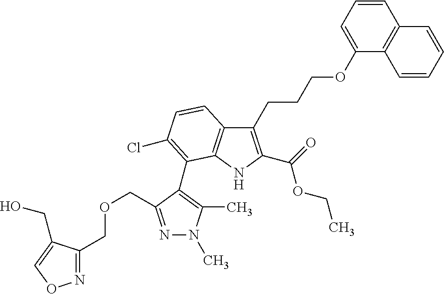 [SMILES: CCOC(=O)c1[nH]c2c(-c3c(COCc4nocc4CO)nn(C)c3C)c(Cl)ccc2c1CCCOc1cccc2ccccc12]

Ethyl-6-chloro-3-{3-[(naphthalen-1-yl)oxy]propyl}-7-(4,4,5,5-tetramethyl-1,3,2-dioxaborolan-2-yl)-1H-indole-2-carboxylate (see Intermediate 5, 332 mg, 621 μmol), (3-{[(4-bromo-1,5-dimethyl-1H-pyrazol-3-yl)methoxy]methyl}-1,2-oxazol-4-yl)methanol (see Intermediate 112, 196 mg, 621 μmol) and potassium phosphate (281 mg, 1.24 mmol) were suspended in a mixture of 5.6 mL of toluene and 560 μL of water and degassed for 20 minutes. XPhos Pd G3 (52.6 mg, 62.1 μmol) was added and the reaction was heated at 110° C. for 20 minutes by microwave irradiation. The mixture was filtered through celite, washed with ethyl acetate. Water was added and the mixture was extracted with ethyl acetate. The combined organics were washed with brine, dried over sodium sulfate and concentrated. The crude material was purified by flash chromatography (30 g ZIP Sphere silica cartridge), eluting a 0-20% methanol in dichloromethane gradient, to give 143 mg of the title compound (54% purity). The material was used in the next step without further purification.

UPLC1-MS (CSH C18 long acid 50-95%): Rt=3.24 min, MS (ESIpos): [M+H]+ 643.

### Intermediate 114

### (rac)-ethyl 3-chloro-4,5-dimethyl-16-{3-[(naphthalen-1-yl)oxy]propyl}-5,7-dihydro-9H,13H-[1,2]oxazolo[3′,4′:3,4]pyrazolo[4′,3′:9,10][1,6]oxazacycloundecino[8,7,6-hi]indole-15-carboxylate

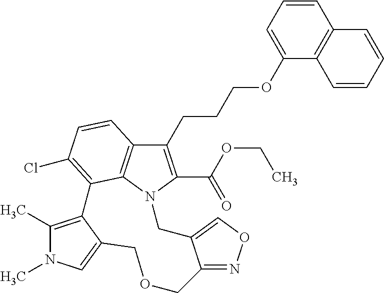 [SMILES: CCOC(=O)c1c(CCCOc2cccc3ccccc23)c2ccc(Cl)c3c2n1Cc1conc1COCc1cn(C)c(C)c1-3]

Di-tert-butyl (E)-diazene-1,2-dicarboxylate (410 mg, 1.78 mmol) was added to ethyl 6-chloro-7-[3-({[4-(hydroxymethyl)-1,2-oxazol-3-yl]methoxy}methyl)-1,5-dimethyl-1H-pyrazol-4-yl]-3-{3-[(naphthalen-1-yl)oxy]propyl}-1H-indole-2-carboxylate (see Intermediate 113, 143 mg, 222 μmol) and triphenylphosphine (467 mg, 1.78 mmol) in 15 mL of tetrahydrofuran and stirred for 3 days at room temperature. The mixture was concentrated and purified by flash chromatography (C18 ZIP Ultra 120 g), eluting with 30-100% acetonitrile in a 0.1% solution of aqueous formic acid. All fractions eluting after triphenylphosphine were combined and evaporated. The impure product was repurified by Biotage Isolera (C18 30 g), eluting with 30-100% acetonitrile in a 0.1% solution of aqueous formic acid. Appropriate fractions were lyophilised to give 35 mg (26% purity) of the title compound. It was used without further purification.

UPLC1 (CSH C18 long acid 50-95%): Rt=2.19 min, MS (ESIpos): [M+H]+ 625.

### EXAMPLES

### Example 1

### (rac)-4,5-Dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic Acid

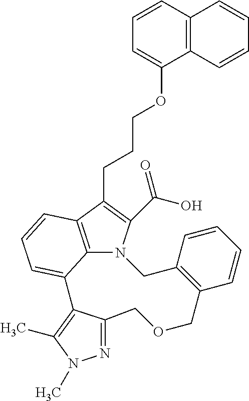 [SMILES: Cc1c2c(nn1C)COCc1ccccc1Cn1c(C(=O)O)c(CCCOc3cccc4ccccc34)c3cccc-2c31]

To a solution of (rac)-ethyl 4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylate (see intermediate 23, 99.0 mg, 165 μmol) in a mixture of THF (2.3 mL) and ethanol (1.2 mL) was added an aqueous solution of lithium hydroxide (1.2 mL, 1.0 M, 1.2 mmol). The resulting mixture was stirred at 50° C. for 1 day, followed by stirring for another day at 65° C. After removal of all volatiles, the residue was subjected to flash chromatography (Biotage SNAP cartridge silica, dichloromethane/ethanol gradient, 0%->10% ethanol) to give the title compound (91 mg).

LC-MS (Method 2): Rt=0.85 min, MS (ESIpos): m/z=573 [M+H]⁺

¹H-NMR (400 MHz, DMSO-d6) δ [ppm]: 0.815 (0.41), 0.822 (0.43), 0.852 (0.46), 0.905 (0.51), 1.036 (5.42), 1.053 (13.28), 1.071 (6.64), 1.233 (2.09), 1.910 (13.20), 2.102 (0.94), 2.119 (1.48), 2.136 (0.97), 2.318 (0.43), 2.337 (0.43), 2.518 (4.88), 2.523 (3.74), 2.660 (0.46), 2.679 (0.43), 3.131 (0.51), 3.145 (0.51), 3.164 (0.89), 3.231 (0.86), 3.250 (0.53), 3.264 (0.61), 3.423 (0.81), 3.435 (0.86), 3.441 (0.84), 3.452 (0.81), 3.810 (16.00), 3.958 (0.94), 3.983 (1.83), 4.029 (1.88), 4.055 (1.20), 4.062 (0.66), 4.071 (0.64), 4.086 (1.14), 4.101 (0.76), 4.114 (1.12), 4.129 (0.59), 4.138 (0.61), 4.173 (1.68), 4.205 (1.98), 4.355 (0.74), 4.366 (2.65), 4.399 (1.86), 5.330 (2.03), 6.793 (1.68), 6.810 (1.83), 6.945 (0.84), 6.963 (1.07), 7.052 (1.02), 7.071 (1.48), 7.081 (1.07), 7.086 (1.45), 7.100 (1.40), 7.103 (1.76), 7.133 (0.56), 7.137 (0.61), 7.151 (1.25), 7.155 (1.35), 7.168 (1.45), 7.173 (1.50), 7.186 (1.22), 7.190 (1.14), 7.204 (0.56), 7.208 (0.51), 7.244 (1.20), 7.261 (0.76), 7.351 (1.42), 7.371 (2.54), 7.390 (2.11), 7.437 (2.47), 7.457 (1.45), 7.472 (0.51), 7.476 (0.71), 7.489 (1.53), 7.493 (1.37), 7.496 (0.69), 7.502 (1.63), 7.509 (2.49), 7.513 (1.65), 7.522 (1.45), 7.527 (1.76), 7.539 (0.74), 7.544 (0.53), 7.677 (1.12), 7.697 (1.04), 7.846 (1.45), 7.852 (0.89), 7.865 (1.65), 7.869 (1.30), 8.155 (0.41), 8.200 (1.32), 8.204 (1.30), 8.222 (1.22), 8.224 (1.25).

The title compound (82 mg) was separated into enantiomers by preparative chiral HPLC to give enantiomer 1 (42 mg, see Example 2) and enantiomer 2 (43 mg, see Example 3).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; gradient: 20-50% B in 20 min.; flow 40.0 mL/min; UV 254 nm

Analytical Chiral HPLC Method

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 2

### 4,5-Dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid-N-ethylethanamine Salt (Enantiomer 1)

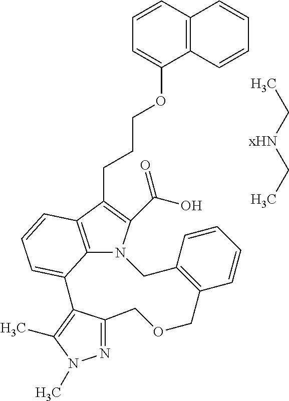

For the preparation of the racemic title compound see Example 1. Separation of enantiomers by preparative chiral HPLC (method see Example 1) gave the title compound (42 mg).

Analytical Chiral HPLC (method see Example 1): Rₜ=2.78 min.

LC-MS (Method 2): Rt=0.82 min; MS (ESIpos): m/z=573 [M+H]⁺

¹H-NMR (400 MHz, DMSO-d6) δ [ppm]: 0.776 (1.12), 0.795 (2.70), 0.814 (1.47), 0.819 (0.60), 0.836 (0.70), 0.844 (0.57), 0.852 (0.52), 0.862 (0.85), 1.006 (2.50), 1.035 (1.02), 1.084 (2.62), 1.131 (6.56), 1.150 (15.23), 1.168 (7.04), 1.204 (0.52), 1.232 (1.27), 1.259 (3.27), 1.287 (0.42), 1.406 (0.42), 1.422 (0.70), 1.425 (0.67), 1.440 (0.65), 1.444 (0.65), 1.866 (12.76), 2.122 (1.12), 2.139 (1.72), 2.155 (1.20), 2.336 (0.47), 2.518 (12.08), 2.522 (9.46), 2.835 (1.65), 2.853 (5.12), 2.872 (4.97), 2.890 (1.62), 3.131 (0.57), 3.145 (0.60), 3.164 (1.02), 3.183 (0.50), 3.198 (0.52), 3.216 (1.02), 3.233 (0.72), 3.249 (0.70), 3.266 (0.47), 3.807 (16.00), 4.028 (0.57), 4.053 (2.32), 4.066 (3.02), 4.091 (1.70), 4.107 (0.57), 4.121 (0.57), 4.138 (1.12), 4.155 (0.65), 4.162 (0.72), 4.182 (1.85), 4.214 (2.20), 4.351 (2.37), 4.383 (1.87), 5.161 (0.62), 5.196 (0.77), 5.551 (0.42), 6.781 (0.80), 6.798 (2.65), 6.815 (2.05), 6.945 (0.80), 6.964 (1.32), 6.982 (0.72), 7.088 (3.32), 7.092 (3.87), 7.100 (2.25), 7.106 (1.67), 7.115 (1.37), 7.344 (1.42), 7.364 (3.24), 7.374 (1.10), 7.384 (2.65), 7.428 (2.62), 7.449 (1.52), 7.467 (0.52), 7.471 (0.67), 7.484 (1.57), 7.488 (1.45), 7.496 (1.72), 7.502 (3.10), 7.508 (1.67), 7.516 (1.57), 7.520 (1.75), 7.533 (0.87), 7.537 (0.70), 7.548 (1.07), 7.568 (0.97), 7.841 (1.55), 7.848 (0.90), 7.860 (1.67), 7.865 (1.30), 8.207 (1.35), 8.212 (1.35), 8.229 (1.25).

### Example 3

### 4,5-Dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid-N-ethylethanamine Salt (Enantiomer 2)

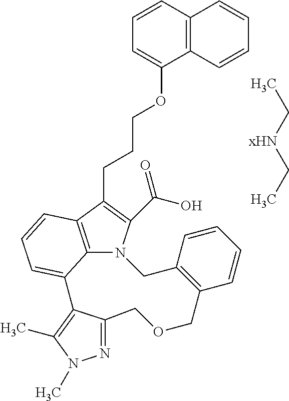

For the preparation of the racemic title compound see Example 1. Separation of enantiomers by preparative chiral HPLC (method see Example 1) gave the title compound (43 mg).

Analytical Chiral HPLC (method see Example 1): Rₜ=5.90 min.

LC-MS (Method 2): Rt=0.83 min, MS (ESIpos): m/z=573 [M+H]⁺

¹H-NMR (400 MHz, DMSO-d6) δ [ppm]: 0.776 (1.11), 0.795 (2.65), 0.814 (1.43), 0.819 (0.59), 0.836 (0.71), 0.844 (0.57), 0.852 (0.47), 0.862 (0.84), 1.006 (2.36), 1.035 (1.06), 1.084 (2.58), 1.133 (5.68), 1.151 (13.20), 1.161 (0.93), 1.169 (6.14), 1.205 (0.47), 1.232 (1.18), 1.259 (3.61), 1.406 (0.39), 1.421 (0.66), 1.425 (0.61), 1.440 (0.61), 1.444 (0.57), 1.867 (12.36), 2.121 (1.08), 2.139 (1.65), 2.155 (1.16), 2.332 (1.03), 2.336 (0.47), 2.518 (11.63), 2.522 (8.87), 2.673 (1.06), 2.678 (0.49), 2.838 (1.28), 2.856 (3.86), 2.874 (3.86), 2.893 (1.25), 3.131 (0.52), 3.145 (0.57), 3.164 (0.96), 3.183 (0.47), 3.199 (0.49), 3.216 (0.98), 3.234 (0.66), 3.249 (0.66), 3.267 (0.44), 3.807 (16.00), 4.026 (0.57), 4.052 (2.24), 4.065 (2.83), 4.090 (1.75), 4.106 (0.57), 4.120 (0.57), 4.137 (1.16), 4.154 (0.71), 4.161 (0.71), 4.181 (1.82), 4.214 (2.14), 4.352 (2.36), 4.384 (1.84), 5.166 (0.59), 5.202 (0.69), 6.798 (2.19), 6.815 (2.11), 6.949 (0.74), 6.968 (1.23), 6.986 (0.69), 7.088 (3.02), 7.094 (3.69), 7.101 (2.09), 7.108 (1.65), 7.117 (1.40), 7.343 (1.40), 7.364 (3.02), 7.383 (2.58), 7.427 (2.51), 7.448 (1.50), 7.466 (0.52), 7.470 (0.66), 7.483 (1.50), 7.487 (1.35), 7.495 (1.70), 7.502 (2.95), 7.507 (1.70), 7.515 (1.57), 7.520 (1.77), 7.532 (0.81), 7.537 (0.61), 7.553 (0.98), 7.573 (0.93), 7.841 (1.47), 7.847 (0.88), 7.859 (1.67), 7.864 (1.30), 8.206 (1.28), 8.211 (1.30), 8.229 (1.23), 8.231 (1.25).

### Example 4

### (rac)-4,5-Dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic Acid

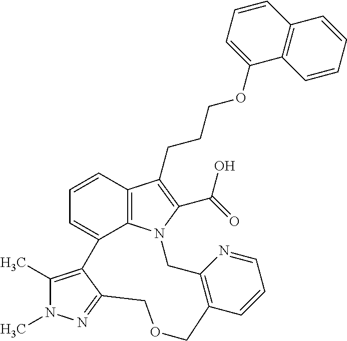 [SMILES: Cc1c2c(nn1C)COCc1cccnc1Cn1c(C(=O)O)c(CCCOc3cccc4ccccc34)c3cccc-2c31]

(rac)-Ethyl 4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylate (see intermediate 24, 49.3 mg, 82.1 μmol) was dissolved in a mixture of 1 mL of THF and 0.2 mL of ethanol and aqueous lithium hydroxide solution (820 μl, 1.0 M, 820 μmol) was added. The reaction mixture was stirred at 70° C. for 3 days and concentrated. The residue was purified by preparative HPLC (Method P2) to obtain the title compound (28.9 mg, 61% yield) as a racemic mixture.

LC-MS (Method 1): Rₜ=1.41 min; MS (ESIpos): m/z=573 [M+H]⁺

¹H-NMR (400 MHz, DMSO-d6) δ [ppm]: 1.857 (4.22), 2.083 (16.00), 2.518 (0.84), 2.523 (0.60), 3.246 (0.63), 3.846 (4.09), 4.082 (0.50), 4.260 (0.46), 4.303 (0.55), 4.335 (0.60), 4.591 (0.42), 5.237 (0.48), 6.830 (0.47), 6.848 (0.51), 6.896 (0.42), 6.898 (0.42), 6.913 (0.56), 6.916 (0.52), 6.996 (0.50), 7.015 (0.62), 7.057 (0.40), 7.069 (0.41), 7.076 (0.41), 7.088 (0.42), 7.388 (0.67), 7.407 (0.58), 7.441 (0.72), 7.500 (0.46), 7.507 (0.60), 7.516 (1.05), 7.519 (0.64), 7.524 (1.01), 7.531 (0.51), 7.538 (0.48), 7.543 (0.57), 7.564 (0.52), 7.566 (0.52), 7.583 (0.49), 7.586 (0.44), 7.849 (0.41), 8.289 (0.51), 8.293 (0.51), 8.301 (0.52), 8.305 (0.47)

The title compound (15.2 mg) was separated into enantiomers by preparative chiral HPLC to give enantiomer 1 (2.3 mg, see Example 5) and enantiomer 2 (2.9 mg, see Example 6).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: 2-propanol; isocratic 50% A+50% B; Flow 50.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: 2-propanol; Gradient: 20-50% B in 7 min; Flow 1.4 mL/min; Temperature: 25° C.; DAD 254 nm

### Example 5

### 4,5-Dimethyl-17-[3-(naphthalen-1-yloxy)propyl]-5,7,9,14-tetrahydropyrazolo[4′,3′:9,10]-pyrido[3′,2′:3,4][1,6]oxazacycloundecino[8,7,6-hi]indole-16-carboxylic Acid (Enantiomer 1)

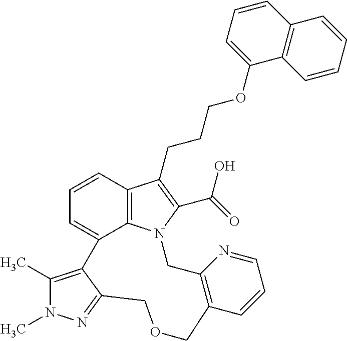 [SMILES: Cc1c2c(nn1C)COCc1cccnc1Cn1c(C(=O)O)c(CCCOc3cccc4ccccc34)c3cccc-2c31]

For the preparation of the racemic title compound see Example 4. Separation of enantiomers by preparative chiral HPLC (method see Example 4) gave the title compound (2.3 mg).

Analytical Chiral HPLC (method see Example 4): Rₜ=4.17 min.

LC-MS (Method 1): Rₜ=1.40 min; MS (ESIpos): m/z=573 [M+H]⁺

¹H-NMR (400 MHz, DMSO-d6) δ [ppm]: 1.073 (3.96), 1.092 (8.45), 1.109 (4.08), 1.230 (1.73), 1.861 (16.00), 2.173 (1.44), 2.331 (1.69), 2.518 (9.69), 2.523 (6.02), 2.539 (2.47), 2.673 (1.73), 2.717 (0.95), 2.735 (2.68), 2.753 (2.60), 2.771 (0.87), 3.249 (3.26), 3.824 (14.76), 4.082 (1.77), 4.108 (2.60), 4.127 (1.36), 4.143 (0.62), 4.175 (0.66), 4.191 (1.24), 4.208 (0.87), 4.215 (0.87), 4.244 (1.98), 4.269 (1.53), 4.295 (2.14), 4.327 (2.52), 4.501 (1.11), 4.534 (0.87), 5.173 (1.48), 5.210 (1.81), 5.537 (0.82), 5.573 (0.70), 6.832 (2.19), 6.844 (1.81), 6.851 (2.60), 6.860 (1.98), 6.968 (1.65), 6.988 (2.39), 7.006 (1.36), 7.046 (1.61), 7.058 (1.73), 7.065 (1.77), 7.077 (1.81), 7.362 (1.36), 7.382 (2.72), 7.402 (2.19), 7.436 (3.13), 7.457 (1.65), 7.484 (0.70), 7.497 (1.81), 7.504 (2.39), 7.512 (5.07), 7.521 (2.72), 7.529 (3.13), 7.534 (2.19), 7.540 (2.76), 7.559 (1.90), 7.847 (1.73), 7.856 (0.91), 7.864 (1.40), 7.871 (1.44), 8.224 (1.53), 8.230 (1.36), 8.248 (1.40), 8.267 (1.94), 8.271 (1.94), 8.278 (1.94), 8.283 (1.73).

### Example 6

### 4,5-Dimethyl-17-[3-(naphthalen-1-yloxy)propyl]-5,7,9,14-tetrahydropyrazolo[4′,3′:9,10]-pyrido[3′,2′:3,4][1,6]oxazacycloundecino[8,7,6-hi]indole-16-carboxylic Acid (Enantiomer 2)

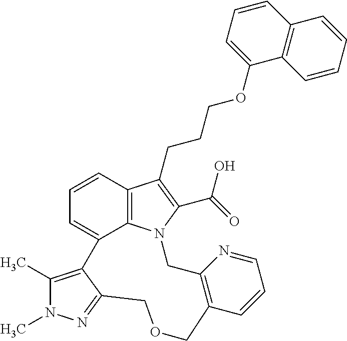 [SMILES: Cc1c2c(nn1C)COCc1cccnc1Cn1c(C(=O)O)c(CCCOc3cccc4ccccc34)c3cccc-2c31]

For the preparation of the racemic title compound see Example 4. Separation of enantiomers by preparative chiral HPLC (method see Example 4) gave the title compound (2.9 mg).

Analytical Chiral HPLC (method see Example 4): Rₜ=7.31 min.

LC-MS (Method 1): Rₜ=1.40 min; MS (ESIpos): m/z=573 [M+H]⁺

¹H-NMR (400 MHz, DMSO-d6) δ [ppm]: 1.063 (3.80), 1.080 (7.88), 1.099 (3.89), 1.231 (1.50), 1.862 (16.00), 2.174 (1.36), 2.190 (1.03), 2.518 (12.15), 2.522 (7.32), 2.692 (0.84), 2.710 (2.30), 2.729 (2.21), 2.746 (0.75), 3.249 (2.82), 3.268 (2.72), 3.821 (14.12), 4.086 (1.74), 4.112 (2.58), 4.129 (1.27), 4.145 (0.52), 4.176 (0.61), 4.192 (1.22), 4.208 (0.75), 4.216 (0.84), 4.242 (1.92), 4.268 (1.45), 4.294 (2.06), 4.326 (2.49), 4.492 (0.89), 4.523 (0.70), 5.170 (1.41), 5.207 (1.74), 5.547 (0.66), 5.583 (0.56), 6.834 (2.63), 6.853 (3.24), 6.966 (1.60), 6.985 (2.30), 7.003 (1.27), 7.046 (1.55), 7.057 (1.64), 7.064 (1.69), 7.076 (1.69), 7.362 (1.36), 7.382 (2.67), 7.402 (2.21), 7.436 (3.05), 7.457 (1.60), 7.484 (0.66), 7.497 (1.78), 7.504 (2.39), 7.512 (5.07), 7.521 (2.72), 7.528 (3.38), 7.540 (2.63), 7.557 (1.83), 7.847 (1.74), 7.856 (0.89), 7.864 (1.41), 7.871 (1.45), 8.224 (1.50), 8.230 (1.31), 8.248 (1.45), 8.265 (1.88), 8.270 (1.88), 8.276 (1.83), 8.281 (1.69).

### Example 7

### (rac)-4,5-Dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic Acid

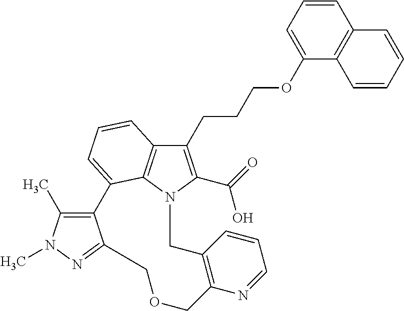 [SMILES: Cc1c2c(nn1C)COCc1ncccc1Cn1c(C(=O)O)c(CCCOc3cccc4ccccc34)c3cccc-2c31]

(rac)-Ethyl 4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylate (see intermediate 25, 71.1 mg, 118 μmol) was dissolved in a mixture of 650 μL of THF and 350 μL of ethanol, and aqueous lithium hydroxide solution (1.2 mL, 1.0 M, 1.2 mmol) was added. The reaction mixture was stirred at 70° C. for 3 days and concentrated. The residue was purified by preparative HPLC (Method P2) to obtain the title compound (47.9 mg, 71% yield) as a racemic mixture.

LC-MS (Method 1): Rₜ=1.33 min, MS (ESIpos): m/z=573 [M+H]⁺

¹H-NMR (400 MHz, DMSO-d6) δ [ppm]: 1.928 (9.47), 2.074 (0.44), 2.100 (0.71), 2.117 (1.11), 2.134 (0.71), 2.327 (0.61), 2.331 (0.43), 2.518 (2.33), 2.523 (1.60), 2.539 (0.63), 2.669 (0.61), 2.673 (0.43), 3.150 (0.53), 3.165 (0.59), 3.184 (0.89), 3.203 (0.60), 3.226 (0.74), 3.244 (1.26), 3.263 (1.30), 3.278 (1.69), 3.492 (0.43), 3.779 (0.43), 3.797 (10.06), 4.017 (1.00), 4.041 (1.41), 4.077 (0.50), 4.092 (0.93), 4.113 (0.91), 4.130 (0.45), 4.169 (1.39), 4.193 (1.16), 4.222 (1.14), 4.253 (1.46), 4.398 (1.60), 4.431 (1.23), 5.369 (1.91), 5.377 (1.73), 6.798 (1.22), 6.816 (1.30), 6.985 (1.11), 6.988 (1.14), 7.003 (1.56), 7.006 (1.45), 7.080 (1.30), 7.100 (1.54), 7.118 (0.94), 7.229 (1.07), 7.241 (1.05), 7.248 (1.07), 7.260 (1.10), 7.353 (0.86), 7.374 (1.63), 7.393 (1.32), 7.438 (1.73), 7.459 (1.05), 7.478 (0.44), 7.491 (1.02), 7.495 (0.92), 7.504 (1.08), 7.510 (1.87), 7.515 (1.17), 7.524 (1.02), 7.528 (1.16), 7.540 (0.54), 7.603 (1.03), 7.607 (1.05), 7.622 (0.96), 7.626 (0.91), 7.708 (1.23), 7.711 (1.25), 7.728 (1.18), 7.731 (1.11), 7.847 (1.04), 7.853 (0.62), 7.865 (1.16), 7.870 (0.93), 8.139 (16.00), 8.199 (0.91), 8.204 (0.92), 8.222 (0.89), 8.326 (1.23), 8.330 (1.26), 8.338 (1.25), 8.342 (1.10)

The title compound (30 mg) was separated into enantiomers by preparative chiral HPLC to give enantiomer 1 (5.7 mg, see Example 8) and enantiomer 2 (7.2 mg, see Example 9).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; isocratic 40% B; Flow 50.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 8

### 4,5-Dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid-N-ethylethanamine Salt (Enantiomer 1)

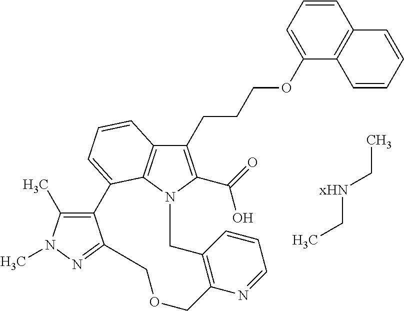

For the preparation of the racemic title compound see Example 7. Separation of enantiomers by preparative chiral HPLC (method see Example 7) gave the title compound (5.7 mg).

Analytical Chiral HPLC (method see Example 7): Rₜ=3.52 min.

LC-MS (Method 1): Rₜ=1.31 min, MS (ESIpos): m/z=573 [M+H]⁺

¹H-NMR (400 MHz, DMSO-d6) δ [ppm]: 1.134 (6.97), 1.152 (16.00), 1.170 (7.05), 1.230 (0.95), 1.891 (13.31), 2.109 (1.06), 2.126 (1.62), 2.144 (1.10), 2.332 (0.75), 2.518 (4.14), 2.522 (2.83), 2.673 (0.78), 2.851 (1.73), 2.869 (5.46), 2.888 (5.31), 2.905 (1.64), 3.121 (0.63), 3.136 (0.71), 3.154 (1.06), 3.172 (0.63), 3.195 (0.76), 3.212 (1.32), 3.230 (1.16), 3.245 (1.32), 3.800 (15.57), 4.052 (0.67), 4.060 (0.65), 4.077 (2.26), 4.102 (2.13), 4.126 (1.08), 4.143 (0.60), 4.150 (0.67), 4.180 (2.09), 4.204 (1.51), 4.239 (1.42), 4.271 (1.94), 4.381 (2.44), 4.413 (1.81), 5.175 (0.80), 5.212 (0.97), 5.592 (0.69), 5.628 (0.62), 6.788 (1.77), 6.805 (2.44), 6.819 (1.34), 6.956 (1.17), 6.975 (1.72), 6.994 (0.99), 7.136 (1.06), 7.148 (1.14), 7.156 (1.14), 7.168 (1.10), 7.342 (1.25), 7.362 (2.35), 7.382 (1.85), 7.425 (2.57), 7.446 (1.47), 7.464 (0.48), 7.468 (0.62), 7.482 (1.51), 7.485 (1.32), 7.494 (1.62), 7.500 (2.87), 7.506 (1.60), 7.514 (1.47), 7.518 (1.64), 7.531 (0.75), 7.535 (0.56), 7.548 (1.29), 7.568 (1.19), 7.789 (1.01), 7.807 (0.93), 7.839 (1.55), 7.846 (0.90), 7.858 (1.62), 7.863 (1.31), 8.203 (1.31), 8.208 (1.32), 8.227 (1.23), 8.268 (1.55), 8.272 (1.59), 8.279 (1.59), 8.283 (1.44).

### Example 9

### 4,5-Dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid-N-ethylethanamine Salt (Enantiomer 2)

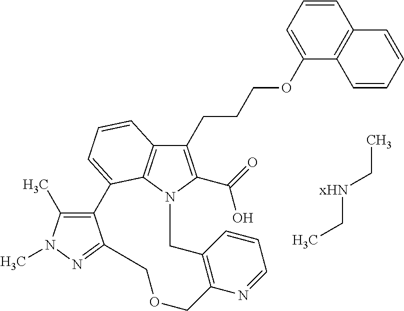

For the preparation of the racemic title compound see Example 7. Separation of enantiomers by preparative chiral HPLC (method see Example 7) gave the title compound (7.2 mg).

Analytical Chiral HPLC (method see Example 7): Rₜ=6.74 min.

LC-MS (Method 1): Rₜ=1.31 min, MS (ESIpos): m/z=573 [M+H]⁺

¹H-NMR (400 MHz, DMSO-d6) δ [ppm]: 1.128 (7.20), 1.147 (16.00), 1.164 (7.40), 1.230 (1.13), 1.256 (0.43), 1.259 (0.43), 1.890 (12.43), 1.904 (0.43), 2.109 (0.99), 2.126 (1.51), 2.144 (1.04), 2.332 (0.93), 2.336 (0.41), 2.518 (5.12), 2.522 (3.45), 2.673 (0.93), 2.678 (0.43), 2.841 (1.72), 2.859 (5.15), 2.877 (5.12), 2.895 (1.60), 3.117 (0.52), 3.131 (0.56), 3.150 (0.88), 3.169 (0.47), 3.191 (0.54), 3.209 (1.02), 3.226 (0.79), 3.242 (0.86), 3.786 (0.47), 3.802 (14.17), 4.053 (0.63), 4.060 (0.61), 4.082 (1.49), 4.093 (0.56), 4.106 (2.01), 4.128 (0.99), 4.145 (0.56), 4.152 (0.63), 4.181 (1.96), 4.206 (1.40), 4.238 (1.38), 4.270 (1.85), 4.380 (2.28), 4.413 (1.62), 5.164 (0.72), 5.200 (0.88), 5.608 (0.63), 5.644 (0.56), 6.789 (2.53), 6.807 (2.75), 6.948 (1.02), 6.967 (1.56), 6.985 (0.88), 7.134 (0.99), 7.145 (1.04), 7.153 (1.04), 7.165 (1.04), 7.344 (1.15), 7.364 (2.21), 7.383 (1.72), 7.426 (2.39), 7.446 (1.38), 7.465 (0.45), 7.469 (0.59), 7.482 (1.38), 7.486 (1.24), 7.495 (1.49), 7.501 (2.69), 7.506 (1.49), 7.514 (1.40), 7.518 (1.51), 7.532 (1.04), 7.536 (1.56), 7.556 (1.08), 7.797 (0.90), 7.816 (0.86), 7.840 (1.47), 7.846 (0.86), 7.858 (1.53), 7.863 (1.20), 8.205 (1.24), 8.209 (1.22), 8.229 (1.17), 8.266 (1.47), 8.270 (1.47), 8.278 (1.51), 8.282 (1.33).

### Example 10

### (rac)-4,5-Dimethyl-19-[3-(naphthalen-1-yloxy)propyl]-5,7,9,16-tetrahydroindolo[1′,7′:6,7,8]-pyrazolo[4′,3′:9,10][1,6]oxazacycloundecino[3,4-b]quinoxaline-18-carboxylic Acid

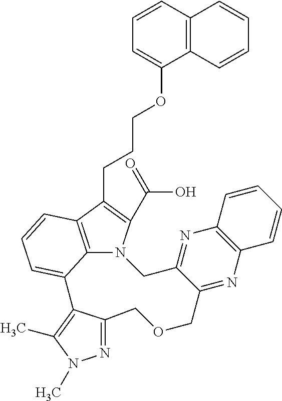 [SMILES: Cc1c2c(nn1C)COCc1nc3ccccc3nc1Cn1c(C(=O)O)c(CCCOc3cccc4ccccc34)c3cccc-2c31]

To a solution of (rac)-ethyl 4,5-dimethyl-19-[3-(naphthalen-1-yloxy)propyl]-5,7,9,16-tetrahydroindolo[1′,7′:6,7,8]pyrazolo[4′,3′:9,10][1,6]oxazacycloundecino[3,4-b]quinoxaline-18-carboxylate (see intermediate 26, 52.0 mg, 79.8 μmol) in a mixture of THF (1.6 mL) and ethanol (0.78 mL) was added an aqueous solution of lithium hydroxide (0.78 mL, 1.0 M, 0.78 mmol). The resulting mixture was stirred at 40° C. for 5 days, followed by stirring for 16 hours at 50° C. and one day at ambient temperature. After removal of all volatiles, the residue was subjected to flash chromatography (Biotage SNAP cartridge silica, dichloromethane/methanol gradient, 0%->25% methanol) to give enriched product, which was re-chromatographed on a preparative HPLC (Method P4), which was run in a basic mode with 0.05% ammonia (32%) instead of the formic acid. This procedure yielded the title compound (14 mg).

LC-MS (Method 2): Rₜ=0.82 min, MS (ESIpos): m/z=625 [M+H]⁺

¹H-NMR (400 MHz, DMSO-d6) δ [ppm]: 0.852 (0.57), 1.233 (1.70), 1.912 (16.00), 2.126 (0.76), 2.145 (1.13), 2.163 (1.20), 2.182 (0.82), 2.200 (0.57), 2.331 (2.71), 2.518 (15.37), 2.522 (9.64), 2.539 (1.20), 2.560 (0.44), 2.585 (0.76), 2.673 (2.71), 3.198 (0.50), 3.213 (0.82), 3.232 (0.94), 3.248 (0.57), 3.265 (0.63), 3.283 (1.07), 3.790 (0.44), 3.843 (4.60), 4.066 (0.88), 4.075 (0.76), 4.090 (1.26), 4.106 (0.57), 4.135 (0.63), 4.151 (1.39), 4.166 (0.88), 4.175 (0.94), 4.340 (0.76), 4.362 (2.20), 4.393 (1.70), 4.679 (1.51), 4.702 (1.45), 4.729 (0.76), 4.762 (0.63), 5.351 (1.20), 5.388 (1.70), 5.574 (1.39), 5.612 (1.07), 6.735 (2.20), 6.754 (2.33), 6.960 (1.26), 6.977 (2.02), 7.018 (2.14), 7.037 (2.71), 7.056 (1.39), 7.293 (1.83), 7.313 (3.15), 7.332 (2.20), 7.419 (3.09), 7.440 (2.27), 7.484 (0.76), 7.497 (2.02), 7.503 (2.96), 7.512 (4.41), 7.521 (3.15), 7.527 (2.46), 7.539 (0.82), 7.562 (2.27), 7.565 (2.27), 7.582 (2.08), 7.585 (2.08), 7.717 (0.57), 7.729 (1.95), 7.733 (2.58), 7.743 (3.40), 7.753 (3.02), 7.757 (2.39), 7.769 (0.76), 7.842 (1.95), 7.851 (1.07), 7.859 (1.51), 7.865 (1.70), 7.922 (2.71), 7.925 (2.83), 7.934 (1.95), 7.940 (1.89), 7.945 (2.27), 7.949 (2.02), 8.199 (1.70), 8.206 (1.39), 8.215 (0.82), 8.224 (1.64).

### Example 11

### (rac)-4,5-Dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic Acid

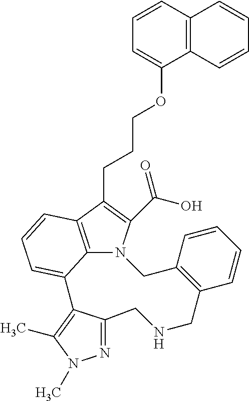 [SMILES: Cc1c2c(nn1C)CNCc1ccccc1Cn1c(C(=O)O)c(CCCOc3cccc4ccccc34)c3cccc-2c31]

To a solution of (rac)-ethyl 4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 31, 100 mg, 167 μmol) in a mixture of THF (5.0 mL) and ethanol (2.5 mL) was added an aqueous solution of lithium hydroxide (2.5 mL, 1.0 M, 2.5 mmol). The resulting mixture was stirred at 45° C. for three days. After removal of all volatiles, the residue was subjected to flash chromatography (Biotage SNAP cartridge silica, dichloromethane/ethanol gradient, 0%->25% ethanol) to give the title compound (89 mg).

LC-MS (Method 2): Rt=0.82 min, MS (ESIpos): m/z=572 [M+H]⁺

¹H-NMR (400 MHz, DMSO-d6) δ [ppm]: 0.798 (1.69), 0.803 (0.75), 0.814 (1.82), 0.821 (1.85), 0.840 (0.88), 0.886 (0.96), 0.904 (1.98), 0.922 (0.91), 1.035 (2.63), 1.052 (5.98), 1.070 (3.00), 1.231 (1.47), 1.756 (16.00), 1.907 (0.62), 2.190 (0.51), 2.202 (1.31), 2.210 (1.13), 2.221 (1.77), 2.239 (1.29), 2.255 (0.43), 2.331 (1.13), 2.336 (0.51), 2.413 (0.46), 2.518 (6.19), 2.523 (4.07), 2.673 (1.15), 2.678 (0.51), 3.271 (2.01), 3.294 (4.45), 3.308 (3.51), 3.331 (6.30), 3.374 (2.63), 3.411 (0.83), 3.428 (1.58), 3.441 (2.04), 3.445 (1.98), 3.464 (0.80), 3.471 (1.15), 3.602 (1.77), 3.638 (1.47), 4.129 (0.78), 4.137 (0.78), 4.153 (1.37), 4.170 (0.78), 4.191 (1.34), 4.207 (0.75), 4.215 (0.75), 5.229 (0.99), 5.267 (1.26), 5.623 (0.72), 5.661 (0.62), 5.760 (0.51), 6.770 (1.47), 6.787 (1.74), 6.842 (2.20), 6.860 (2.33), 6.941 (1.61), 6.948 (3.56), 6.954 (3.97), 6.967 (2.76), 6.985 (1.42), 7.013 (0.99), 7.019 (0.64), 7.032 (1.37), 7.045 (0.70), 7.053 (0.75), 7.161 (2.14), 7.179 (1.74), 7.354 (1.66), 7.374 (3.08), 7.393 (2.44), 7.436 (3.22), 7.457 (1.82), 7.479 (0.48), 7.483 (0.72), 7.496 (1.93), 7.500 (1.80), 7.504 (2.22), 7.512 (4.21), 7.520 (2.30), 7.524 (1.98), 7.528 (2.12), 7.541 (0.78), 7.545 (0.48), 7.588 (1.82), 7.607 (1.69), 7.848 (1.85), 7.856 (0.99), 7.866 (1.66), 7.871 (1.58), 8.242 (1.66), 8.248 (1.53), 8.258 (0.83), 8.266 (1.55).

The title compound (82 mg) was separated into enantiomers by preparative chiral HPLC to give enantiomer 1 (28 mg, see Example 12) and enantiomer 2 (29 mg, see Example 13).

Preparative Chiral HPLC Method:

Instrument: Labomatic HD5000, Labocord-5000; Gilson GX-241, Labcol Vario 4000, column: Chiralpak ID 5μ 250×30 mm; Eluent A: hexane+0.1 Vol-% trifluoroacetic acid; Eluent B: 2-propanol; isocratic: 60% A+40% B; flow 50.0 mL/min; UV 254 nm

Analytical Chiral HPLC Method:

Instrument: Agilent HPLC 1260; Säule: Chiralpak ID 3μ 100×4.6 mm; Eluent A: hexane+0.1 Vol-% trifluoroacetic acid; Eluent B: 2-propanol; isocratic 60% A+40% B; flow 1.4 mL/min; Temperature: 25° C.; DAD 254 nm

### Example 12

### 4,5-Dimethyl-17-[3-(naphthalen-1-yloxy)propyl]-7,8,9,14-tetrahydro-5H-indolo[1,7-bc]pyrazolo-[4,3-e][2,8]benzodiazacycloundecine-16-carboxylic acid-Trifluoroacetic Acid Salt (Enantiomer 1)

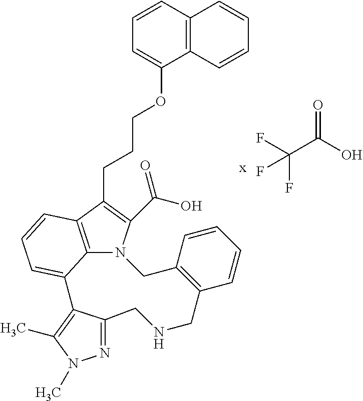 [SMILES: Cc1c2c(nn1C)CNCc1ccccc1Cn1c(C(=O)O)c(CCCOc3cccc4ccccc34)c3cccc-2c31 ; O=C(O)C(F)(F)F]

For the preparation of the racemic title compound see Example 11. Separation of enantiomers by preparative chiral HPLC (method see Example 11) gave the title compound (28 mg).

Analytical Chiral HPLC (method see Example 11): Rₜ=2.92 min.

LC-MS (Method 2): Rₜ=0.81 min; MS (ESIpos): m/z=571 [M+H]⁺

¹H-NMR (400 MHz, DMSO-d6) δ [ppm]: 0.851 (0.43), 1.137 (0.55), 1.154 (0.63), 1.233 (1.41), 1.410 (2.75), 1.754 (0.51), 2.336 (0.94), 2.518 (16.00), 2.522 (10.43), 2.539 (1.61), 3.889 (3.45), 4.173 (1.41), 4.207 (1.41), 6.879 (0.78), 7.097 (0.75), 7.235 (0.82), 7.368 (1.10), 7.388 (2.12), 7.407 (1.65), 7.453 (2.78), 7.474 (1.69), 7.490 (0.71), 7.504 (1.41), 7.508 (1.37), 7.517 (1.80), 7.523 (2.71), 7.527 (1.65), 7.537 (1.61), 7.540 (1.69), 7.553 (0.71), 7.785 (1.41), 7.806 (1.29), 7.861 (1.65), 7.879 (1.69), 7.884 (1.37), 8.250 (0.59).

### Example 13

### 4,5-Dimethyl-17-[3-(naphthalen-1-yloxy)propyl]-7,8,9,14-tetrahydro-5H-indolo[1,7-bc]pyrazolo-[4,3-e][2,8]benzodiazacycloundecine-16-carboxylic acid-Trifluoroacetic Acid Salt (Enantiomer 2)

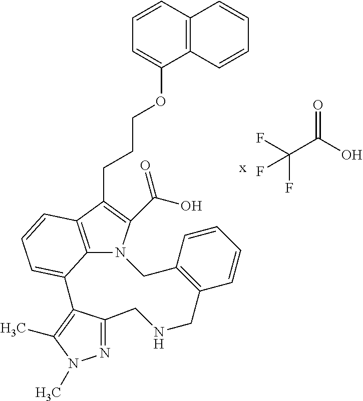 [SMILES: Cc1c2c(nn1C)CNCc1ccccc1Cn1c(C(=O)O)c(CCCOc3cccc4ccccc34)c3cccc-2c31 ; O=C(O)C(F)(F)F]

For the preparation of the racemic title compound see Example 11. Separation of enantiomers by preparative chiral HPLC (method see Example 11) gave the title compound (29 mg).

Analytical Chiral HPLC (method see Example 11): Rₜ=4.34 min.

LC-MS (Method 2): Rₜ=0.81 min; MS (ESIpos): m/z=571 [M+H]⁺

¹H-NMR (400 MHz, DMSO-d6) δ [ppm]: 0.852 (0.39), 1.155 (0.51), 1.233 (1.45), 1.411 (2.10), 1.765 (0.56), 2.269 (0.43), 2.337 (0.98), 2.518 (16.00), 2.523 (10.48), 2.540 (4.92), 2.679 (0.81), 3.890 (4.41), 4.171 (1.75), 4.206 (1.80), 5.171 (0.47), 6.880 (0.90), 7.097 (0.77), 7.234 (0.98), 7.369 (1.33), 7.389 (2.52), 7.408 (1.93), 7.454 (3.21), 7.475 (1.97), 7.491 (0.77), 7.504 (1.67), 7.508 (1.58), 7.518 (2.01), 7.523 (3.17), 7.528 (1.93), 7.537 (1.80), 7.541 (2.01), 7.554 (0.86), 7.558 (0.64), 7.787 (1.67), 7.806 (1.58), 7.861 (1.88), 7.880 (1.93), 7.884 (1.63), 8.262 (0.68).

### Example 14

### (rac)-4,5,8-Trimethyl-17-[3-(naphthalen-1-yloxy) propyl]-7,8,9,14-tetrahydro-5H-indolo[1,7-bc]pyrazolo[4,3-e][2,8]benzodiazacycloundecine-16-carboxylic Acid

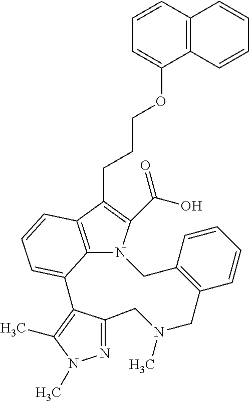 [SMILES: Cc1c2c(nn1C)CN(C)Cc1ccccc1Cn1c(C(=O)O)c(CCCOc3cccc4ccccc34)c3cccc-2c31]

To a solution of (rac)-ethyl 4,5,8-trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 32, 147 mg, 240 μmol) in a mixture of THF (6.4 mL) and ethanol (3.2 mL) was added an aqueous solution of lithium hydroxide (3.2 mL, 1.0 M, 3.2 mmol). The resulting mixture was stirred at ambient temperature for four days, then at 70° C. for seven hours followed by stirring at 60° C. for 16 hours and finally at 90° C. for four hours. After removal of all volatiles, the residue was subjected to flash chromatography (Biotage SNAP cartridge silica, dichloromethane/ethanol gradient, 0%->100% ethanol) to give enriched material which was subjected to a second flash chromatography (Biotage SNAP cartridge silica, dichloromethane/ethanol gradient, 0%->70% ethanol) to give the title compound (63 mg).

LC-MS (Method 2): Rt=0.94 min, MS (ESIneg): m/z=585 [M+H]⁺

¹H-NMR (400 MHz, DMSO-d6) δ [ppm]: 0.798 (5.44), 0.803 (2.49), 0.815 (6.04), 0.822 (6.08), 0.840 (3.06), 0.851 (0.85), 0.860 (0.53), 0.877 (0.53), 0.886 (3.13), 0.905 (6.26), 0.922 (3.20), 1.035 (0.57), 1.053 (1.28), 1.071 (0.85), 1.108 (0.78), 1.124 (0.60), 1.142 (0.78), 1.161 (0.57), 1.169 (0.68), 1.205 (0.96), 1.230 (3.95), 1.256 (1.14), 1.270 (0.60), 1.274 (0.64), 1.289 (0.43), 1.758 (13.40), 1.799 (0.53), 1.816 (0.46), 1.905 (0.75), 2.084 (0.39), 2.153 (2.70), 2.178 (16.00), 2.203 (1.49), 2.210 (1.49), 2.230 (1.00), 2.336 (0.71), 2.359 (0.75), 2.373 (0.92), 2.388 (1.14), 2.394 (1.21), 2.399 (0.60), 2.406 (1.14), 2.412 (1.53), 2.430 (0.43), 2.518 (9.21), 2.523 (6.04), 2.678 (0.68), 3.034 (0.85), 3.064 (1.14), 3.207 (1.56), 3.225 (1.74), 3.242 (1.85), 3.259 (2.17), 3.277 (1.85), 3.323 (8.85), 3.334 (9.00), 3.356 (7.15), 3.429 (0.53), 3.446 (0.43), 3.529 (2.77), 3.562 (2.13), 4.068 (0.57), 4.085 (1.32), 4.092 (1.35), 4.109 (2.10), 4.125 (1.07), 4.136 (1.07), 4.153 (1.99), 4.169 (1.28), 4.176 (1.28), 4.193 (0.53), 5.153 (0.96), 5.188 (1.14), 5.624 (0.46), 5.760 (5.83), 6.762 (1.53), 6.778 (1.81), 6.798 (2.92), 6.817 (2.92), 6.919 (1.53), 6.937 (2.67), 6.949 (2.42), 6.961 (3.20), 6.972 (3.31), 7.014 (3.41), 7.023 (3.73), 7.340 (1.71), 7.360 (4.37), 7.379 (3.38), 7.427 (5.30), 7.447 (3.20), 7.468 (1.00), 7.473 (1.35), 7.485 (3.45), 7.490 (3.59), 7.495 (5.12), 7.502 (8.53), 7.509 (4.66), 7.515 (4.94), 7.520 (5.48), 7.532 (1.85), 7.537 (1.21), 7.841 (3.45), 7.849 (1.96), 7.860 (3.34), 7.865 (2.95), 8.214 (2.77), 8.219 (2.77), 8.237 (2.77).

The title compound (55 mg) was separated into enantiomers by preparative chiral HPLC to give enantiomer 1 (20 mg, see Example 15) and enantiomer 2 (25 mg, see Example 16).

Preparative Chiral HPLC Method:

Instrument: Labomatic HD5000, Labocord-5000; Gilson GX-241, Labcol Vario 4000, column: Chiralpak ID 5μ 250×30 mm; Eluent A: hexane+0.1 Vol-% N-ethylethanamine (99%); Eluent B: ethanol; Gradient: 20-50% B in 20 min; Flow 40.0 mL/min; UV 254 nm

Analytical Chiral HPLC Method:

Instrument: Agilent HPLC 1260; column: Chiralpak ID 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 15

### 4,5,8-Trimethyl-17-[3-(naphthalen-1-yloxy)propyl]-7,8,9,14-tetrahydro-5H-indolo[1,7-bc]-pyrazolo[4,3-e][2,8]benzodiazacycloundecine-16-carboxylic acid-N-ethylethanamine Salt (Enantiomer 1)

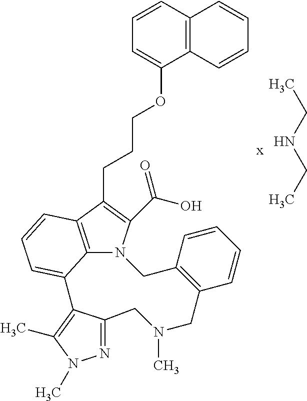

For the preparation of the racemic title compound see Example 14. Separation of enantiomers by preparative chiral HPLC (method see Example 14) gave the title compound (25 mg).

Analytical Chiral HPLC (method see Example 14): Rₜ=2.62 min.

LC-MS (Method 2): Rₜ=0.93 min, MS (ESIpos): m/z=585 [M+H]⁺

¹H-NMR (400 MHz, DMSO-d6) δ [ppm]: 0.851 (0.43), 1.107 (0.69), 1.149 (7.04), 1.167 (16.00), 1.185 (7.09), 1.232 (2.24), 1.787 (10.00), 2.125 (0.93), 2.151 (6.53), 2.176 (0.59), 2.332 (1.09), 2.336 (0.48), 2.518 (5.63), 2.523 (3.97), 2.539 (15.92), 2.678 (0.48), 2.692 (0.67), 2.862 (1.97), 2.881 (6.32), 2.899 (5.97), 2.917 (1.87), 3.095 (1.60), 3.152 (0.53), 3.166 (0.53), 3.186 (0.83), 3.204 (0.43), 3.232 (0.56), 3.250 (0.99), 3.268 (0.91), 3.283 (1.33), 3.509 (1.41), 3.542 (1.07), 3.781 (12.91), 4.076 (0.53), 4.083 (0.53), 4.100 (0.91), 4.117 (0.48), 4.138 (0.85), 4.154 (0.45), 4.162 (0.48), 5.207 (0.45), 5.244 (0.59), 6.798 (1.60), 6.816 (1.92), 6.947 (1.01), 6.955 (1.20), 6.960 (1.15), 6.970 (2.05), 6.989 (0.53), 7.043 (1.68), 7.052 (1.60), 7.057 (1.55), 7.066 (1.41), 7.320 (0.59), 7.347 (1.25), 7.368 (2.08), 7.387 (1.68), 7.432 (2.11), 7.453 (1.23), 7.476 (0.56), 7.489 (1.33), 7.494 (1.20), 7.501 (1.41), 7.507 (2.85), 7.513 (1.52), 7.520 (1.31), 7.524 (1.49), 7.537 (1.31), 7.558 (0.72), 7.844 (1.28), 7.851 (0.72), 7.862 (1.33), 7.867 (1.07), 8.215 (1.12), 8.220 (1.07), 8.239 (1.09).

### Example 16

### 4,5,8-Trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic Acid (Enantiomer 2)

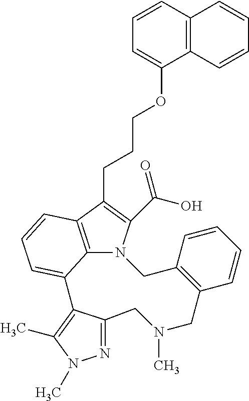 [SMILES: Cc1c2c(nn1C)CN(C)Cc1ccccc1Cn1c(C(=O)O)c(CCCOc3cccc4ccccc34)c3cccc-2c31]

For the preparation of the racemic title compound see Example 14. Separation of enantiomers by preparative chiral HPLC (method see Example 14) gave the title compound (20 mg).

Analytical Chiral HPLC (method see Example 14): Rₜ=4.14 min.

LC-MS (Method 2): Rₜ=0.92 min, MS (ESIpos): m/z=585 [M+H]⁺

¹H-NMR (400 MHz, DMSO-d6) δ [ppm]: 1.107 (1.11), 1.137 (3.15), 1.155 (6.65), 1.173 (3.24), 1.232 (1.23), 1.766 (11.60), 1.905 (0.63), 2.135 (1.05), 2.152 (1.71), 2.179 (7.79), 2.332 (1.26), 2.336 (0.57), 2.518 (6.41), 2.523 (4.46), 2.539 (4.58), 2.673 (1.20), 2.678 (0.51), 2.831 (0.69), 2.848 (2.10), 2.867 (2.10), 2.885 (0.66), 3.050 (0.51), 3.081 (0.87), 3.157 (0.93), 3.171 (0.84), 3.189 (1.17), 3.209 (0.60), 3.223 (0.66), 3.240 (1.14), 3.257 (0.96), 3.272 (1.08), 3.514 (1.74), 3.547 (1.35), 3.779 (16.00), 4.083 (0.66), 4.090 (0.60), 4.107 (1.05), 4.123 (0.45), 4.136 (0.48), 4.152 (1.02), 4.169 (0.57), 4.176 (0.60), 5.135 (0.54), 5.171 (0.63), 6.732 (0.66), 6.749 (0.72), 6.807 (1.71), 6.824 (1.86), 6.901 (0.72), 6.920 (1.14), 6.944 (1.11), 6.950 (0.75), 6.956 (1.14), 6.967 (1.59), 7.013 (1.86), 7.022 (2.13), 7.033 (1.38), 7.346 (1.41), 7.366 (2.73), 7.385 (2.70), 7.428 (2.61), 7.448 (1.50), 7.469 (0.66), 7.473 (1.14), 7.478 (1.05), 7.486 (1.98), 7.490 (1.86), 7.497 (2.52), 7.504 (3.90), 7.510 (1.83), 7.516 (1.56), 7.520 (1.68), 7.533 (0.69), 7.537 (0.42), 7.841 (1.53), 7.848 (0.84), 7.859 (1.53), 7.865 (1.26), 8.217 (1.35), 8.222 (1.26), 8.241 (1.26).

### Example 17

### (rac)-4,5-Dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic Acid Acetate Salt

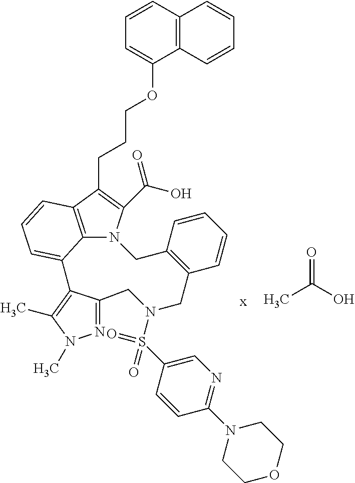

To a solution of (rac)-ethyl 4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 34, 183 mg, 222 μmol) in a mixture of THF (5.0 mL) and ethanol (2.5 mL) was added an aqueous solution of lithium hydroxide (2.5 mL, 1.0 M, 2.5 mmol). The resulting mixture was stirred at 80° C. for five hours followed by stirring at ambient temperature for three days. After addition of acetic acid (1 mL) all volatiles were removed and the residue subjected to flash chromatography (Biotage SNAP cartridge silica, dichloromethane/ethanol gradient, 0%->20% ethanol) to give the title compound (174 mg).

LC-MS (Method 2): Rₜ=0.98 min, MS (ESIpos): m/z=798 [M+H]⁺

¹H-NMR (400 MHz, DMSO-d6) δ [ppm]: 0.901 (0.74), 1.035 (0.57), 1.053 (1.03), 1.070 (0.57), 1.232 (1.10), 1.612 (5.43), 1.907 (16.00), 2.065 (1.04), 2.283 (0.54), 2.298 (0.41), 2.518 (2.70), 2.522 (1.76), 3.348 (0.76), 3.367 (1.00), 3.385 (0.52), 3.453 (0.74), 3.492 (0.70), 3.543 (0.61), 3.587 (7.20), 3.653 (2.00), 3.666 (2.11), 3.703 (2.23), 3.717 (2.13), 3.727 (1.05), 4.191 (0.71), 4.208 (0.73), 4.662 (0.56), 4.700 (0.52), 5.069 (0.53), 5.109 (0.64), 5.547 (0.41), 5.758 (0.45), 6.762 (0.76), 6.777 (0.85), 6.867 (0.88), 6.884 (0.94), 6.939 (0.67), 6.960 (0.48), 6.969 (1.70), 6.989 (1.51), 7.007 (0.70), 7.089 (0.43), 7.107 (0.76), 7.367 (0.84), 7.374 (0.76), 7.387 (1.48), 7.393 (0.71), 7.406 (1.12), 7.450 (1.32), 7.471 (0.76), 7.509 (0.84), 7.513 (0.78), 7.516 (1.00), 7.525 (1.78), 7.533 (1.05), 7.536 (0.89), 7.540 (0.91), 7.675 (0.86), 7.677 (0.89), 7.695 (0.82), 7.698 (0.80), 7.859 (0.76), 7.868 (0.42), 7.877 (0.63), 7.882 (0.67), 7.988 (0.45), 7.994 (0.47), 8.011 (0.43), 8.017 (0.43), 8.266 (0.67), 8.272 (0.62), 8.290 (0.64), 8.549 (0.79), 8.554 (0.79).

The title compound (166 mg) was separated into enantiomers by preparative chiral HPLC to give enantiomer 1 (114 mg, see Example 18) and enantiomer 2 (69 mg, see Example 19).

Preparative Chiral HPLC Method:

Instrument: Labomatic HD5000, Labocord-5000; Gilson GX-241, Labcol Vario 4000,

column: YMC Cellulose SB 250×30 mm; Eluent A: hexane+0.1 Vol-% trifluoroacetic acid (99%); Eluent B: 2-propanol; isocratic 50% A+50% B; flow 40.0 mL/min; UV 280 nm

Analytical Chiral HPLC Method:

Instrument: Waters Alliance 2695Agilent HPLC 1260; column: YMC Cellulose SB 3μ 100×4.6 mm; Eluent A: hexane+0.1 Vol-% trifluoroacetic acid (99%); Eluent B: 2-propanol; isocratic 50% B; flow 1.4 mL/min; Temperature: 25° C.; DAD 280 nm

### Example 18

### (+)-4,5-Dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic Acid (Enantiomer 1)

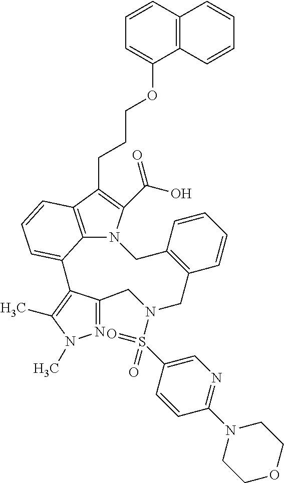 [SMILES: Cc1c2c(nn1C)CN(S(=O)(=O)c1ccc(N3CCOCC3)nc1)Cc1ccccc1Cn1c(C(=O)O)c(CCCOc3cccc4ccccc34)c3cccc-2c31]

For the preparation of the racemic title compound see Example 17. Separation of enantiomers by preparative chiral HPLC (method see Example 17) gave the title compound (114 mg).

Analytical Chiral HPLC (method see Example 17): Rₜ=3.31 min.

LC-MS (Method 2): Rₜ=0.94 min, MS (ESIpos): m/z=797 [M+H]⁺

Specific Optical Rotation (Method O1): +11.2° (c=10 mg/mL, CHCl₃)

¹H-NMR (400 MHz, DMSO-d6) δ [ppm]: 1.027 (0.58), 1.042 (0.58), 1.236 (0.71), 1.316 (10.14), 1.331 (11.22), 1.613 (11.08), 2.282 (1.15), 2.518 (16.00), 2.523 (12.17), 2.660 (0.58), 3.350 (1.08), 3.369 (2.03), 3.387 (1.12), 3.454 (1.39), 3.493 (1.49), 3.544 (1.39), 3.566 (0.54), 3.654 (4.34), 3.667 (4.64), 3.704 (4.81), 3.717 (4.75), 3.727 (2.54), 4.175 (1.66), 4.192 (2.58), 4.208 (2.64), 4.223 (1.83), 4.232 (1.66), 4.283 (1.83), 4.318 (1.59), 4.662 (1.32), 4.700 (1.22), 5.073 (1.19), 5.114 (1.49), 5.160 (0.58), 5.176 (0.75), 5.191 (0.58), 5.539 (1.08), 5.579 (0.98), 6.472 (0.78), 6.491 (0.88), 6.765 (1.66), 6.767 (1.80), 6.783 (2.00), 6.785 (2.03), 6.867 (1.80), 6.885 (1.93), 6.925 (0.75), 6.943 (1.42), 6.972 (2.98), 6.992 (4.00), 7.010 (1.63), 7.091 (0.92), 7.109 (1.63), 7.129 (0.88), 7.367 (1.59), 7.376 (1.59), 7.388 (3.08), 7.407 (2.27), 7.451 (2.71), 7.472 (1.63), 7.492 (0.41), 7.497 (0.61), 7.509 (1.59), 7.514 (1.53), 7.518 (2.07), 7.526 (3.69), 7.534 (2.14), 7.537 (1.86), 7.542 (1.97), 7.554 (0.75), 7.559 (0.51), 7.680 (1.90), 7.682 (2.07), 7.700 (1.83), 7.702 (1.86), 7.860 (1.53), 7.868 (0.88), 7.877 (1.36), 7.883 (1.42), 7.988 (0.92), 7.994 (1.02), 8.011 (0.95), 8.017 (0.98), 8.266 (1.29), 8.272 (1.25), 8.282 (0.71), 8.290 (1.39), 8.547 (1.66), 8.553 (1.73).

### Example 19

### (−)-4,5-Dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic Acid (Enantiomer 2)

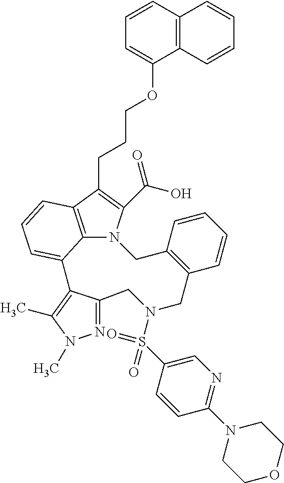 [SMILES: Cc1c2c(nn1C)CN(S(=O)(=O)c1ccc(N3CCOCC3)nc1)Cc1ccccc1Cn1c(C(=O)O)c(CCCOc3cccc4ccccc34)c3cccc-2c31]

For the preparation of the racemic title compound see Example 17. Separation of enantiomers by preparative chiral HPLC (method see Example 17) gave the title compound (69 mg).

Analytical Chiral HPLC (method see Example 17): Rₜ=5.45 min.

LC-MS (Method 2): Rₜ=0.92 min, MS (ESIpos): m/z=797 [M+H]⁺

Specific Optical Rotation (Method O1): −14.8° (c=10 mg/mL, CHCl₃)

¹H-NMR (400 MHz, DMSO-d6) δ [ppm]: 1.219 (0.49), 1.236 (1.08), 1.613 (11.04), 2.283 (1.15), 2.318 (0.94), 2.337 (0.77), 2.518 (16.00), 2.523 (12.33), 2.679 (0.66), 3.350 (1.29), 3.369 (2.24), 3.386 (1.57), 3.454 (1.96), 3.493 (2.55), 3.544 (4.40), 3.654 (5.21), 3.667 (5.28), 3.704 (5.17), 3.717 (4.93), 3.727 (2.69), 4.167 (0.52), 4.175 (0.73), 4.192 (1.57), 4.208 (1.61), 4.223 (0.77), 4.233 (0.56), 4.282 (0.80), 4.318 (0.73), 4.662 (1.19), 4.701 (1.08), 5.073 (1.19), 5.114 (1.47), 5.539 (1.08), 5.579 (0.98), 6.471 (0.80), 6.491 (0.87), 6.765 (1.75), 6.767 (1.82), 6.783 (2.06), 6.785 (2.03), 6.867 (1.82), 6.885 (1.96), 6.925 (0.73), 6.943 (1.47), 6.972 (2.69), 6.992 (4.05), 7.010 (1.61), 7.091 (0.91), 7.109 (1.61), 7.128 (0.87), 7.368 (1.75), 7.375 (1.61), 7.388 (3.11), 7.407 (2.31), 7.452 (2.76), 7.472 (1.64), 7.493 (0.42), 7.497 (0.66), 7.510 (1.68), 7.514 (1.61), 7.518 (2.06), 7.526 (3.77), 7.534 (2.20), 7.537 (1.85), 7.542 (1.92), 7.554 (0.73), 7.559 (0.45), 7.680 (1.99), 7.682 (2.06), 7.700 (1.89), 7.703 (1.85), 7.860 (1.57), 7.868 (0.87), 7.877 (1.40), 7.884 (1.40), 7.988 (0.94), 7.993 (0.98), 8.010 (0.94), 8.016 (0.94), 8.266 (1.36), 8.272 (1.29), 8.282 (0.73), 8.290 (1.36), 8.547 (1.68), 8.553 (1.71).

### Example 20

### (rac)-4,5-Dimethyl-17-[3-(naphthalen-1-yloxy)propyl]-8-[(3,4,5-trimethoxybenzyl)carbamoyl]-7,8,9,14-tetrahydro-5H-indolo[1,7-bc]pyrazolo[4,3-e][2,8]benzodiazacycloundecine-16-carboxylic Acid

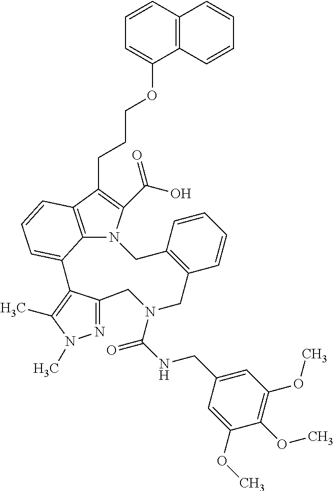

To a solution of (rac)-ethyl 4,5-dimethyl-17-[3-(naphthalen-1-yloxy)propyl]-8-[(3,4,5-trimethoxybenzyl)carbamoyl]-7,8,9,14-tetrahydro-5H-indolo[1,7-bc]pyrazolo[4,3-e][2,8]benzodiazacycloundecine-16-carboxylate (see intermediate 33, 155 mg, 189 μmol) in a mixture of THF (5.0 mL) and ethanol (2.5 mL) was added an aqueous solution of lithium hydroxide (2.5 mL, 1.0 M, 2.5 mmol). The resulting mixture was stirred at 80° C. for five hours followed by stirring at ambient temperature for 3 days. After addition of acetic acid (1 mL), all volatiles were removed and the residue was subjected to flash chromatography (Biotage SNAP cartridge silica, dichloromethane/ethanol gradient, 0%->25% ethanol) to give the title compound (105 mg).

LC-MS (Method 2): Rₜ=0.95 min, MS (ESIpos): m/z=795 [M+H]⁺

¹H-NMR (400 MHz, DMSO-d6) δ [ppm]: 1.233 (0.46), 1.700 (4.17), 1.907 (5.72), 2.318 (0.48), 2.322 (0.68), 2.326 (0.85), 2.332 (0.65), 2.518 (2.46), 2.522 (1.60), 2.664 (0.41), 2.668 (0.57), 2.673 (0.41), 3.377 (0.46), 3.396 (0.68), 3.632 (11.92), 3.765 (16.00), 3.797 (5.23), 4.225 (0.63), 4.240 (0.86), 4.255 (0.54), 5.759 (2.27), 6.649 (3.62), 6.770 (0.55), 6.786 (0.62), 6.893 (0.89), 6.910 (1.18), 6.991 (0.57), 7.010 (0.92), 7.029 (0.84), 7.195 (0.62), 7.214 (0.59), 7.373 (0.55), 7.394 (0.95), 7.413 (0.79), 7.454 (0.99), 7.475 (0.57), 7.513 (0.60), 7.517 (0.61), 7.520 (0.83), 7.528 (1.33), 7.537 (0.86), 7.539 (0.71), 7.544 (0.70), 7.705 (0.58), 7.722 (0.55), 7.862 (0.57), 7.865 (0.44), 7.880 (0.44), 7.886 (0.50), 8.276 (0.51), 8.283 (0.45), 8.300 (0.48).

### Example 21

### (rac)-4,5-Dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic Acid

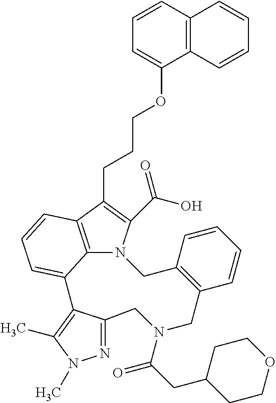 [SMILES: Cc1c2c(nn1C)CN(C(=O)CC1CCOCC1)Cc1ccccc1Cn1c(C(=O)O)c(CCCOc3cccc4ccccc34)c3cccc-2c31]

To a solution of (rac)-ethyl 4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylate (see intermediate 35, 175 mg, 241 μmol) in a mixture of THF (5.0 mL) and ethanol (2.5 mL) was added an aqueous solution of lithium hydroxide (2.5 mL, 1.0 M, 2.5 mmol). The resulting mixture was stirred at ambient temperature for three days followed by stirring at 50° C. for two hours. After addition of acetic acid (1 mL), all volatiles were removed and the residue was subjected to flash chromatography (Biotage SNAP cartridge silica, dichloromethane/ethanol gradient, 0%->30% ethanol) to give the title compound (145 mg).

LC-MS (Method 2): Rₜ=0.93 min, MS (ESIpos): m/z=698 [M+H]⁺

¹H-NMR (400 MHz, DMSO-d6) δ [ppm]: 0.798 (0.59), 0.815 (0.62), 0.821 (0.59), 0.840 (0.42), 0.851 (0.42), 0.904 (0.59), 1.035 (2.88), 1.053 (5.90), 1.070 (2.98), 1.182 (1.15), 1.203 (1.42), 1.232 (2.98), 1.256 (1.46), 1.286 (0.59), 1.589 (1.32), 1.644 (1.56), 1.702 (13.99), 1.765 (0.80), 1.907 (14.61), 2.023 (0.94), 2.322 (3.51), 2.326 (3.75), 2.373 (0.49), 2.421 (2.64), 2.437 (2.46), 2.669 (2.08), 3.259 (1.70), 3.288 (3.33), 3.406 (2.85), 3.423 (2.36), 3.452 (1.18), 3.475 (1.74), 3.510 (2.88), 3.551 (1.49), 3.730 (2.01), 3.787 (16.00), 3.810 (2.95), 3.934 (0.42), 3.967 (0.45), 4.214 (1.60), 4.229 (3.57), 4.244 (3.51), 4.258 (1.60), 4.355 (0.73), 4.622 (1.32), 4.660 (1.21), 5.137 (0.83), 5.173 (0.83), 5.328 (1.46), 5.370 (1.84), 5.682 (1.42), 5.723 (1.15), 6.482 (1.53), 6.502 (1.63), 6.774 (0.59), 6.796 (2.46), 6.815 (2.50), 6.896 (3.68), 6.915 (4.72), 6.931 (1.74), 6.946 (1.01), 6.993 (2.19), 7.012 (3.51), 7.030 (2.05), 7.043 (0.94), 7.063 (2.46), 7.079 (4.96), 7.163 (1.04), 7.373 (2.12), 7.393 (4.48), 7.413 (3.26), 7.454 (5.24), 7.475 (2.95), 7.520 (4.03), 7.530 (4.55), 7.539 (4.30), 7.556 (0.97), 7.715 (2.85), 7.735 (2.19), 7.863 (3.02), 7.879 (2.36), 7.886 (2.46), 8.281 (2.22), 8.305 (1.74).

The title compound (137 mg) was separated into enantiomers by preparative chiral HPLC to give enantiomer 1 (84 mg, see Example 22) and enantiomer 2 (83 mg, see Example 23).

Preparative Chiral HPLC Method:

Instrument: Labomatic HD5000, Labocord-5000; Gilson GX-241, Labcol Vario 4000, column: YMC Cellulose SC 250×30 mm; Eluent A: hexane+0.1 Vol-% trifluoroacetic acid (99%); Eluent B: 2-propanol; isocratic 50% A+50% B; flow 40.0 mL/min; UV 280 nm

Analytical Chiral HPLC Method:

Instrument: Waters Alliance 2695Agilent HPLC 1260; column Säule: YMC Cellulose SC 3μ 100×4.6 mm; Eluent A: hexane+0.1 Vol-% trifluoroacetic acid (99%); Eluent B: 2-propanol; isocratic 50% B; flow 1.4 mL/min; Temperature: 25° C.; DAD 280 nm

### Example 22

### (−)-4,5-Dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic Acid (Enantiomer 1)

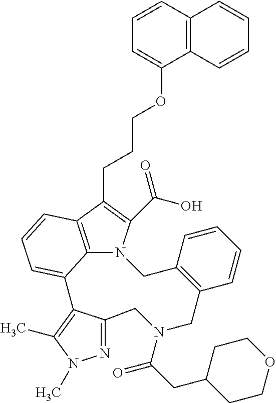 [SMILES: Cc1c2c(nn1C)CN(C(=O)CC1CCOCC1)Cc1ccccc1Cn1c(C(=O)O)c(CCCOc3cccc4ccccc34)c3cccc-2c31]

For the preparation of the racemic title compound see Example 21. Separation of enantiomers by preparative chiral HPLC (method see Example 21) gave the title compound (84 mg).

Analytical Chiral HPLC (method see Example 21): Rₜ=3.01 min.

LC-MS (Method 2): Rₜ=0.88 min, MS (ESIpos): m/z=697 [M+H]⁺

Specific Optical Rotation (Method O1): −13.4° (c=10 mg/mL, CHCl₃)

¹H-NMR (400 MHz, DMSO-d6) δ [ppm]: 1.027 (0.52), 1.042 (0.48), 1.144 (0.41), 1.171 (0.71), 1.181 (0.71), 1.202 (0.85), 1.235 (1.56), 1.256 (0.82), 1.267 (0.71), 1.316 (0.71), 1.331 (0.63), 1.363 (0.48), 1.590 (0.82), 1.622 (0.78), 1.645 (0.93), 1.678 (0.85), 1.704 (9.43), 1.762 (0.52), 2.021 (0.52), 2.318 (1.78), 2.323 (2.71), 2.327 (3.19), 2.332 (2.45), 2.336 (1.56), 2.420 (1.41), 2.437 (1.19), 2.518 (16.00), 2.523 (12.62), 2.660 (0.67), 2.665 (1.60), 2.669 (2.26), 2.673 (1.60), 2.678 (0.74), 3.257 (1.08), 3.288 (1.82), 3.320 (1.08), 3.377 (0.97), 3.394 (1.41), 3.411 (1.86), 3.428 (1.19), 3.476 (1.45), 3.512 (2.49), 3.553 (2.52), 3.566 (2.60), 3.731 (1.74), 3.787 (10.95), 3.810 (2.00), 4.215 (1.00), 4.231 (2.15), 4.244 (2.12), 4.258 (1.00), 4.620 (0.78), 4.661 (0.74), 5.133 (0.52), 5.167 (0.48), 5.337 (0.97), 5.378 (1.26), 5.666 (1.15), 5.708 (0.97), 6.469 (1.11), 6.489 (1.23), 6.808 (1.71), 6.824 (1.82), 6.898 (2.30), 6.916 (2.97), 6.937 (1.04), 6.951 (0.63), 6.958 (0.63), 6.999 (1.71), 7.019 (2.41), 7.036 (1.52), 7.048 (0.56), 7.067 (1.56), 7.083 (3.23), 7.097 (0.63), 7.166 (0.63), 7.375 (1.71), 7.395 (3.19), 7.414 (2.60), 7.456 (3.49), 7.476 (2.00), 7.504 (0.52), 7.516 (1.63), 7.521 (2.56), 7.531 (3.04), 7.540 (2.82), 7.546 (1.82), 7.558 (0.71), 7.706 (0.45), 7.725 (1.93), 7.743 (1.52), 7.863 (1.93), 7.873 (0.85), 7.881 (1.48), 7.887 (1.63), 8.282 (1.37), 8.289 (1.08), 8.305 (1.04).

### Example 23

### (+)-4,5-Dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic Acid (Enantiomer 2)

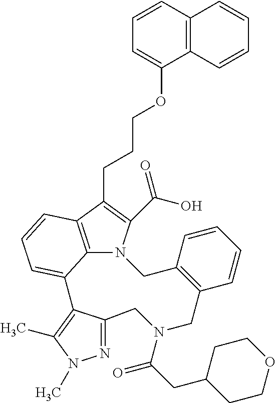 [SMILES: Cc1c2c(nn1C)CN(C(=O)CC1CCOCC1)Cc1ccccc1Cn1c(C(=O)O)c(CCCOc3cccc4ccccc34)c3cccc-2c31]

For the preparation of the racemic title compound see Example 21. Separation of enantiomers by preparative chiral HPLC (method see Example 21) gave the title compound (83 mg).

Analytical Chiral HPLC (method see Example 21): Rₜ=5.37 min.

LC-MS (Method 2): Rₜ=0.88 min, MS (ESIpos): m/z=697 [M+H]⁺

Specific Optical Rotation (Method O1): +14.4° (c=10 mg/mL, CHCl₃)

¹H-NMR (400 MHz, DMSO-d6) δ [ppm]: 0.851 (0.48), 1.026 (0.54), 1.042 (0.54), 1.142 (0.72), 1.172 (1.01), 1.179 (1.07), 1.201 (1.31), 1.234 (2.39), 1.255 (1.19), 1.265 (1.01), 1.284 (0.54), 1.315 (1.01), 1.330 (1.01), 1.588 (1.25), 1.643 (1.43), 1.677 (1.43), 1.703 (13.67), 1.767 (0.66), 2.020 (0.78), 2.322 (4.42), 2.326 (5.07), 2.331 (3.94), 2.419 (2.63), 2.436 (2.51), 2.517 (12.84), 2.522 (8.42), 2.664 (2.57), 2.669 (3.46), 2.673 (2.51), 3.259 (1.55), 3.288 (2.87), 3.319 (1.67), 3.393 (2.15), 3.409 (2.87), 3.475 (2.33), 3.511 (3.88), 3.552 (4.12), 3.730 (2.81), 3.787 (16.00), 3.809 (2.99), 3.934 (0.48), 3.968 (0.54), 4.215 (1.55), 4.230 (3.28), 4.243 (3.16), 4.258 (1.49), 4.621 (1.19), 4.661 (1.13), 5.133 (0.78), 5.169 (0.78), 5.336 (1.43), 5.378 (1.85), 5.666 (1.73), 5.707 (1.37), 6.469 (1.73), 6.489 (1.79), 6.782 (0.60), 6.807 (2.63), 6.824 (2.69), 6.897 (3.46), 6.916 (4.42), 6.937 (1.61), 6.952 (0.96), 6.998 (2.33), 7.018 (3.58), 7.036 (2.15), 7.047 (0.90), 7.067 (2.45), 7.082 (4.84), 7.096 (1.01), 7.165 (1.01), 7.375 (2.21), 7.395 (4.42), 7.414 (3.34), 7.455 (5.07), 7.476 (2.87), 7.521 (3.82), 7.531 (4.36), 7.540 (4.06), 7.545 (2.63), 7.558 (0.90), 7.707 (0.72), 7.724 (2.99), 7.744 (2.27), 7.864 (2.81), 7.873 (1.31), 7.881 (2.27), 7.887 (2.33), 8.282 (2.03), 8.305 (1.55), 13.471 (0.42).

### Example 24

### (rac)-4,5-Dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic Acid

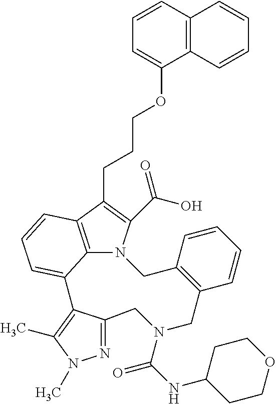 [SMILES: Cc1c2c(nn1C)CN(C(=O)NC1CCOCC1)Cc1ccccc1Cn1c(C(=O)O)c(CCCOc3cccc4ccccc34)c3cccc-2c31]

To a solution of (rac)-ethyl 4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylate (see intermediate 36, 178 mg, 245 μmol) in a mixture of THF (5.1 mL) and ethanol (2.5 mL) was added an aqueous solution of lithium hydroxide (2.5 mL, 1.0 M, 2.5 mmol). The resulting mixture was stirred at 50° C. for 22 hours. After removal of all volatiles, the residue was subjected to flash chromatography (Biotage SNAP cartridge silica, dichloromethane/ethanol gradient, 0%->22% ethanol) to give the title compound (147 mg).

LC-MS (Method 2): Rₜ=0.92 min, MS (ESIpos): m/z=699 [M+H]⁺

¹H-NMR (400 MHz, DMSO-d6) δ [ppm]: 0.814 (0.42), 0.821 (0.42), 0.851 (0.42), 0.904 (0.50), 1.035 (7.62), 1.052 (13.97), 1.070 (8.28), 1.232 (1.77), 1.434 (0.74), 1.443 (0.74), 1.464 (1.08), 1.474 (1.08), 1.496 (0.79), 1.504 (0.74), 1.712 (11.04), 1.739 (1.34), 2.296 (1.24), 2.303 (1.27), 2.318 (1.42), 2.322 (1.85), 2.327 (1.92), 2.331 (1.42), 2.336 (0.82), 2.518 (5.38), 2.523 (3.85), 2.659 (0.50), 2.665 (1.11), 2.669 (1.56), 2.673 (1.08), 2.678 (0.47), 3.308 (1.61), 3.348 (2.48), 3.362 (2.37), 3.372 (1.87), 3.382 (2.53), 3.405 (1.45), 3.417 (0.90), 3.423 (1.92), 3.435 (1.98), 3.440 (1.92), 3.452 (1.90), 3.457 (0.71), 3.469 (0.61), 3.620 (1.00), 3.655 (1.13), 3.684 (0.50), 3.693 (0.58), 3.711 (0.55), 3.799 (16.00), 3.832 (1.16), 3.843 (1.40), 3.861 (1.03), 4.196 (0.53), 4.203 (0.82), 4.219 (1.92), 4.234 (1.85), 4.248 (0.79), 4.257 (0.50), 4.344 (0.95), 4.357 (1.77), 4.370 (0.87), 4.689 (0.50), 4.725 (0.45), 5.323 (0.98), 5.364 (1.21), 5.580 (0.84), 5.622 (0.66), 6.418 (0.61), 6.431 (0.61), 6.511 (0.79), 6.530 (0.84), 6.772 (1.74), 6.788 (1.98), 6.889 (2.16), 6.900 (0.98), 6.906 (2.48), 6.917 (1.50), 6.936 (0.82), 6.992 (1.71), 7.011 (2.24), 7.029 (1.53), 7.041 (0.95), 7.060 (1.71), 7.079 (0.92), 7.186 (1.95), 7.189 (2.00), 7.206 (1.56), 7.373 (1.61), 7.394 (2.95), 7.413 (2.40), 7.454 (3.08), 7.474 (1.71), 7.496 (0.45), 7.500 (0.69), 7.513 (1.95), 7.518 (1.92), 7.520 (2.56), 7.529 (4.14), 7.537 (2.66), 7.544 (2.11), 7.557 (0.71), 7.561 (0.42), 7.698 (1.87), 7.716 (1.74), 7.863 (1.77), 7.871 (0.92), 7.880 (1.34), 7.886 (1.50), 8.274 (1.53), 8.280 (1.37), 8.290 (0.74), 8.298 (1.45), 13.433 (0.50).

The title compound (140 mg) was separated into enantiomers by preparative chiral HPLC to give enantiomer 1 (71 mg, see Example 25) and enantiomer 2 (75 mg, see Example 26).

Preparative Chiral HPLC Method:

Instrument: Labomatic HD5000, Labocord-5000; Gilson GX-241, Labcol Vario 4000, column: Chiralpak IA 250×30 mm; Eluent A: hexane+0.1 Vol-% trifluoroacetic acid (99%); Eluent B: 2-propanol; isocratic 50% A+50% B; Flow 40.0 mL/min; UV 280 nm

Analytical Chiral HPLC Method:

Instrument: Waters Alliance 2695Agilent HPLC 1260; column: Chiralpak IA 3μ 100×4.6 mm; Eluent A: hexane+0.1 Vol-% trifluoroacetic acid (99%); Eluent B: 2-propanol; isocratic 50% B; Flow 1.4 mL/min; Temperature: 25° C.; DAD 280 nm

### Example 25

### (+)-4,5-Dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic Acid (Enantiomer 1)

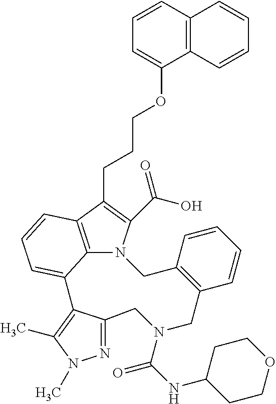 [SMILES: Cc1c2c(nn1C)CN(C(=O)NC1CCOCC1)Cc1ccccc1Cn1c(C(=O)O)c(CCCOc3cccc4ccccc34)c3cccc-2c31]

For the preparation of the racemic title compound see Example 24. Separation of enantiomers by preparative chiral HPLC (method see Example 24) gave the title compound (71 mg).

Analytical Chiral HPLC (method see Example 24): Rₜ=1.71 min.

LC-MS (Method 2): Rₜ=0.84 min, MS (ESIpos): m/z=698 [M+H]⁺

Specific Optical Rotation (Method O1): +63.9° (c=10 mg/mL, CHCl₃)

¹H-NMR (400 MHz, DMSO-d6) δ [ppm]: 1.108 (16.00), 1.715 (2.61), 2.323 (0.48), 2.327 (0.53), 2.518 (1.79), 2.523 (1.15), 2.669 (0.44), 3.332 (0.41), 3.337 (0.43), 3.343 (0.45), 3.349 (0.46), 3.365 (0.56), 3.384 (0.61), 3.832 (0.77), 3.842 (0.82), 3.860 (0.67), 4.219 (0.47), 4.233 (0.46), 6.776 (0.49), 6.779 (0.52), 6.794 (0.57), 6.796 (0.57), 6.889 (0.49), 6.906 (0.61), 6.996 (0.48), 7.015 (0.59), 7.064 (0.42), 7.191 (0.48), 7.395 (0.68), 7.414 (0.55), 7.455 (0.76), 7.476 (0.42), 7.514 (0.42), 7.521 (0.58), 7.530 (0.90), 7.538 (0.61), 7.545 (0.48), 7.701 (0.50), 7.703 (0.53), 7.721 (0.48), 7.724 (0.48), 7.863 (0.40).

### Example 26

### (−)-4,5-Dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic Acid (Enantiomer 2)

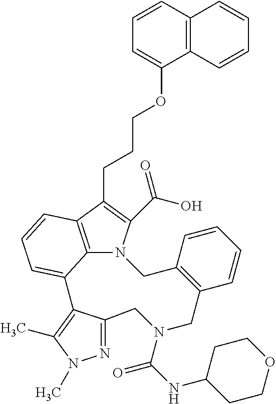 [SMILES: Cc1c2c(nn1C)CN(C(=O)NC1CCOCC1)Cc1ccccc1Cn1c(C(=O)O)c(CCCOc3cccc4ccccc34)c3cccc-2c31]

For the preparation of the racemic title compound see Example 24. Separation of enantiomers by preparative chiral HPLC (method see Example 24) gave the title compound (75 mg).

Analytical Chiral HPLC (method see Example 24): Rₜ=3.13 min.

LC-MS (Method 2): Rₜ=0.84 min, MS (ESIpos): m/z=698 [M+H]⁺

Specific Optical Rotation (Method O1): −75.2° (c=10 mg/mL, CHCl₃)

¹H-NMR (400 MHz, DMSO-d6) δ [ppm]: 1.107 (16.00), 1.715 (2.34), 2.323 (0.46), 2.327 (0.53), 2.518 (2.78), 2.523 (1.78), 2.669 (0.42), 3.349 (0.41), 3.365 (0.50), 3.384 (0.54), 4.204 (0.42), 4.219 (0.65), 4.233 (0.65), 4.247 (0.45), 6.776 (0.46), 6.779 (0.46), 6.794 (0.52), 6.796 (0.51), 6.889 (0.44), 6.906 (0.55), 6.996 (0.43), 7.015 (0.52), 7.191 (0.42), 7.394 (0.60), 7.413 (0.49), 7.455 (0.64), 7.518 (0.40), 7.521 (0.52), 7.529 (0.83), 7.538 (0.55), 7.545 (0.43), 7.701 (0.45), 7.703 (0.47), 7.721 (0.44), 7.724 (0.42).

### Example 27

### (rac)-4,5-Dimethyl-17-[3-(naphthalen-1-yloxy)propyl]-8-[(tetrahydro-2H-pyran-4-ylmethyl)-carbamoyl]-7,8,9,14-tetrahydro-5H-indolo[1,7-bc]pyrazolo[4,3-e][2,8]benzodiazacycloundecine-16-carboxylic Acid

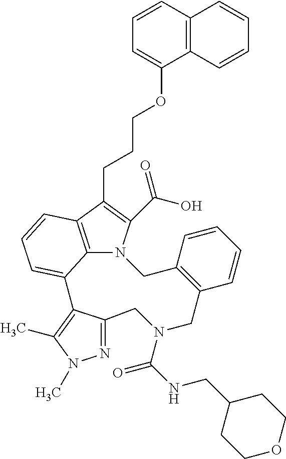 [SMILES: Cc1c2c(nn1C)CN(C(=O)NCC1CCOCC1)Cc1ccccc1Cn1c(C(=O)O)c(CCCOc3cccc4ccccc34)c3cccc-2c31]

To a solution of (rac)-ethyl 4,5-dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzo-diazacycloundecine-16-carboxylate (see intermediate 37, 170 mg, 230 μmol) in a mixture of THF (4.8 mL) and ethanol (2.4 mL) was added an aqueous solution of lithium hydroxide (2.4 mL, 1.0 M, 2.4 mmol). The resulting mixture was stirred at ambient temperature for 17 hours, followed by stirring at 65° C. for five hours. After removal of all volatiles, the residue was subjected to flash chromatography (Biotage SNAP cartridge silica, dichloromethane/ethanol gradient, 0%->22% ethanol) to give the title compound (132 mg).

LC-MS (Method 2): Rₜ=0.93 min, MS (ESIpos): m/z=713 [M+H]⁺

¹H-NMR (400 MHz, DMSO-d6) δ [ppm]: 0.814 (0.42), 0.821 (0.45), 0.904 (0.49), 1.035 (4.12), 1.052 (7.64), 1.070 (4.45), 1.142 (1.17), 1.172 (1.19), 1.194 (0.47), 1.201 (0.52), 1.211 (0.45), 1.232 (1.43), 1.590 (0.84), 1.621 (1.50), 1.653 (0.77), 1.703 (13.21), 1.722 (0.82), 2.296 (1.27), 2.304 (1.27), 2.318 (1.34), 2.322 (1.78), 2.327 (1.76), 2.331 (1.29), 2.336 (0.77), 2.518 (4.92), 2.523 (3.44), 2.659 (0.47), 2.665 (0.98), 2.669 (1.38), 2.673 (0.98), 2.678 (0.45), 2.884 (0.56), 2.902 (0.89), 2.920 (0.82), 2.934 (0.49), 3.022 (0.47), 3.037 (0.80), 3.053 (0.77), 3.070 (0.59), 3.240 (1.19), 3.247 (0.89), 3.269 (2.30), 3.293 (1.10), 3.299 (1.55), 3.305 (1.29), 3.352 (1.27), 3.365 (1.57), 3.384 (2.44), 3.404 (1.38), 3.417 (0.59), 3.423 (1.12), 3.435 (1.12), 3.440 (1.08), 3.452 (1.05), 3.457 (0.40), 3.618 (1.10), 3.654 (1.15), 3.782 (16.00), 3.840 (1.64), 3.868 (1.45), 4.196 (0.56), 4.204 (0.87), 4.219 (1.94), 4.233 (1.90), 4.248 (0.82), 4.257 (0.49), 4.344 (0.52), 4.357 (0.98), 4.369 (0.49), 4.647 (0.94), 4.684 (0.61), 5.342 (1.01), 5.384 (1.38), 5.587 (1.03), 5.627 (0.80), 6.506 (1.05), 6.525 (1.12), 6.632 (0.80), 6.766 (1.90), 6.768 (1.92), 6.784 (2.13), 6.890 (2.20), 6.901 (1.08), 6.907 (2.48), 6.918 (1.62), 6.936 (0.89), 6.994 (1.90), 7.014 (2.34), 7.032 (1.73), 7.036 (1.12), 7.056 (1.78), 7.074 (0.96), 7.168 (2.01), 7.170 (2.04), 7.187 (1.52), 7.373 (1.66), 7.394 (2.98), 7.413 (2.46), 7.454 (3.14), 7.474 (1.76), 7.495 (0.47), 7.499 (0.73), 7.512 (1.94), 7.516 (1.87), 7.519 (2.44), 7.528 (4.19), 7.536 (2.48), 7.538 (2.04), 7.543 (2.06), 7.555 (0.73), 7.561 (0.42), 7.702 (1.97), 7.704 (1.99), 7.722 (1.83), 7.862 (1.83), 7.871 (0.94), 7.879 (1.48), 7.885 (1.52), 8.273 (1.59), 8.279 (1.43), 8.289 (0.75), 8.297 (1.48), 13.433 (0.63).

The title compound (125 mg) was separated into enantiomers by preparative chiral HPLC to give enantiomer 1 (55 mg, see Example 28) and enantiomer 2 (56 mg, see Example 29).

Preparative Chiral HPLC Method:

Instrument: Labomatic HD5000, Labocord-5000; Gilson GX-241, Labcol Vario 4000, column: Chiralpak IA 250×30 mm; Eluent A: hexane+0.1 Vol-% trifluoroacetic acid (99%); Eluent B: 2-propanol; isocratic 50% A+50% B; Flow 40.0 mL/min; UV 280 nm

Analytical Chiral HPLC Method:

Instrument: Waters Alliance 2695Agilent HPLC 1260; column: Chiralpak IA 3μ 100×4.6 mm; Eluent A: hexane+0.1 Vol-% trifluoroacetic acid (99%); Eluent B: 2-propanol; isocratic 50% B; Flow 1.4 mL/min; Temperature: 25° C.; DAD 280 nm

### Example 28

### (+)-4,5-Dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylic Acid (Enantiomer 1)

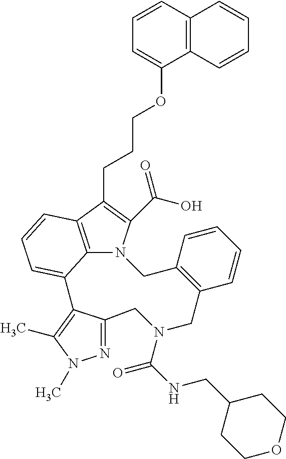 [SMILES: Cc1c2c(nn1C)CN(C(=O)NCC1CCOCC1)Cc1ccccc1Cn1c(C(=O)O)c(CCCOc3cccc4ccccc34)c3cccc-2c31]

For the preparation of the racemic title compound see Example 27. Separation of enantiomers by preparative chiral HPLC (method see Example 27) gave the title compound (55 mg).

Analytical Chiral HPLC (method see Example 27): Rₜ=1.75 min.

LC-MS (Method 2): Rₜ=0.86 min, MS (ESIpos): m/z=712 [M+H]⁺

Specific Optical Rotation (Method O1): +61.5° (c=10 mg/mL, CHCl₃)

¹H-NMR (400 MHz, DMSO-d6) δ [ppm]: 1.027 (0.70), 1.042 (0.67), 1.107 (0.42), 1.142 (1.19), 1.172 (1.23), 1.193 (0.49), 1.201 (0.49), 1.232 (0.67), 1.316 (0.49), 1.331 (0.49), 1.589 (0.81), 1.621 (1.44), 1.653 (0.77), 1.705 (12.28), 2.296 (1.19), 2.304 (1.23), 2.323 (2.14), 2.327 (2.46), 2.332 (1.79), 2.337 (1.02), 2.518 (15.65), 2.523 (12.00), 2.660 (0.60), 2.665 (1.40), 2.669 (1.96), 2.673 (1.44), 2.678 (0.67), 2.884 (0.49), 2.902 (0.77), 2.920 (0.74), 2.933 (0.49), 3.021 (0.42), 3.035 (0.70), 3.052 (0.70), 3.069 (0.53), 3.240 (1.12), 3.246 (0.84), 3.269 (2.14), 3.299 (1.33), 3.316 (0.88), 3.366 (1.51), 3.385 (2.42), 3.404 (1.30), 3.619 (1.23), 3.655 (1.37), 3.782 (16.00), 3.800 (1.79), 3.840 (3.19), 4.204 (0.95), 4.220 (2.00), 4.233 (1.96), 4.249 (0.98), 4.257 (0.63), 4.646 (0.95), 4.684 (0.63), 5.347 (1.05), 5.388 (1.47), 5.579 (1.26), 5.620 (1.02), 6.500 (1.09), 6.520 (1.19), 6.631 (0.74), 6.770 (2.07), 6.773 (2.18), 6.788 (2.39), 6.791 (2.39), 6.890 (2.00), 6.908 (2.46), 6.922 (1.61), 6.942 (0.95), 6.998 (1.96), 7.017 (2.42), 7.036 (1.89), 7.060 (1.72), 7.077 (1.16), 7.172 (1.93), 7.188 (1.47), 7.374 (1.54), 7.394 (2.81), 7.413 (2.28), 7.455 (2.95), 7.475 (1.68), 7.495 (0.42), 7.499 (0.67), 7.512 (1.72), 7.516 (1.68), 7.520 (2.18), 7.528 (3.82), 7.537 (2.28), 7.539 (2.04), 7.544 (2.00), 7.556 (0.81), 7.561 (0.49), 7.706 (2.07), 7.708 (2.18), 7.726 (1.93), 7.729 (1.93), 7.862 (1.68), 7.871 (0.95), 7.880 (1.44), 7.886 (1.51), 8.272 (1.47), 8.279 (1.37), 8.289 (0.77), 8.297 (1.44).

### Example 29

### (−)-4,5-Dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylic Acid (Enantiomer 2)

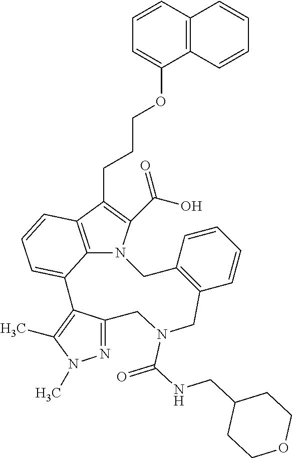 [SMILES: Cc1c2c(nn1C)CN(C(=O)NCC1CCOCC1)Cc1ccccc1Cn1c(C(=O)O)c(CCCOc3cccc4ccccc34)c3cccc-2c31]

For the preparation of the racemic title compound see Example 27. Separation of enantiomers by preparative chiral HPLC (method see Example 27) gave the title compound (56 mg).

Analytical Chiral HPLC (method see Example 27): Rₜ=3.15 min.

LC-MS (Method 2): Rₜ=0.85 min, MS (ESIpos): m/z=712 [M+H]⁺

Specific Optical Rotation (Method O1): −58.8° (c=10 mg/mL, CHCl₃)

¹H-NMR (400 MHz, DMSO-d6) δ [ppm]: 1.107 (1.29), 1.142 (1.09), 1.171 (1.09), 1.201 (0.48), 1.232 (0.56), 1.589 (0.78), 1.621 (1.40), 1.653 (0.73), 1.705 (11.32), 2.304 (1.15), 2.318 (1.32), 2.323 (1.85), 2.327 (2.02), 2.332 (1.51), 2.337 (0.87), 2.518 (13.23), 2.523 (9.64), 2.660 (0.48), 2.665 (1.09), 2.669 (1.57), 2.673 (1.12), 2.884 (0.45), 2.902 (0.76), 2.920 (0.70), 2.933 (0.45), 3.036 (0.67), 3.052 (0.67), 3.069 (0.53), 3.240 (1.06), 3.246 (0.81), 3.269 (2.02), 3.299 (1.23), 3.316 (0.84), 3.366 (1.43), 3.385 (2.24), 3.404 (1.20), 3.619 (1.35), 3.655 (1.60), 3.782 (16.00), 3.800 (2.61), 3.840 (2.61), 3.868 (2.10), 4.204 (0.78), 4.220 (1.74), 4.233 (1.74), 4.247 (0.81), 4.257 (0.53), 4.648 (0.90), 4.681 (0.59), 5.347 (0.98), 5.388 (1.37), 5.579 (1.18), 5.620 (0.92), 6.500 (1.01), 6.520 (1.12), 6.630 (0.70), 6.770 (1.88), 6.773 (2.05), 6.788 (2.19), 6.791 (2.24), 6.890 (1.88), 6.908 (2.27), 6.922 (1.51), 6.942 (0.87), 6.998 (1.79), 7.017 (2.24), 7.036 (1.77), 7.060 (1.63), 7.076 (0.98), 7.172 (1.79), 7.189 (1.37), 7.374 (1.43), 7.394 (2.61), 7.413 (2.10), 7.454 (2.72), 7.475 (1.60), 7.499 (0.64), 7.512 (1.63), 7.516 (1.60), 7.520 (2.02), 7.528 (3.53), 7.537 (2.10), 7.539 (1.91), 7.544 (1.85), 7.556 (0.70), 7.561 (0.42), 7.706 (1.91), 7.708 (2.02), 7.725 (1.79), 7.729 (1.79), 7.862 (1.54), 7.871 (0.87), 7.880 (1.35), 7.886 (1.37), 8.272 (1.35), 8.279 (1.26), 8.289 (0.70), 8.297 (1.35).

### Example 30

### (rac)-4,5-Dimethyl-8-{[2-(morpholin-4-yl)ethyl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic Acid

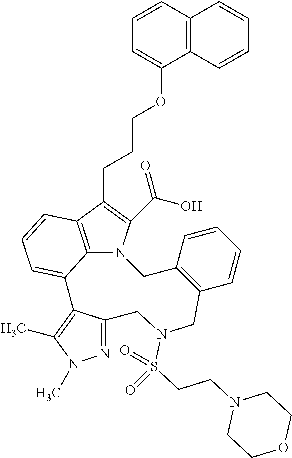 [SMILES: Cc1c2c(nn1C)CN(S(=O)(=O)CCN1CCOCC1)Cc1ccccc1Cn1c(C(=O)O)c(CCCOc3cccc4ccccc34)c3cccc-2c31]

To a solution of (rac)-ethyl 4,5-dimethyl-8-{[2-(morpholin-4-yl)ethyl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylate (see intermediate 39, 147 mg, 189 μmol) in a mixture of THF (5.0 mL) and ethanol (2.5 mL) was added an aqueous solution of lithium hydroxide (2.5 mL, 1.0 M, 2.5 mmol). The resulting mixture was stirred at 70° C. for five hours, followed by stirring at ambient temperature for four days. After removal of all volatiles, the residue was subjected to flash chromatography (Biotage SNAP cartridge silica, dichloromethane/ethanol gradient, 0%->50% ethanol) to give the title compound (133 mg).

LC-MS (Method 2): Rt=0.91 min, MS (ESIneg): m/z=746 [M+H]⁺

¹H-NMR (400 MHz, DMSO-d6) δ [ppm]: 0.821 (0.39), 0.851 (0.42), 0.904 (0.42), 1.232 (2.33), 1.681 (13.53), 2.318 (1.73), 2.323 (2.15), 2.327 (2.57), 2.331 (2.07), 2.518 (6.32), 2.523 (4.59), 2.665 (1.15), 2.669 (1.57), 2.673 (1.10), 2.799 (0.63), 2.817 (0.89), 2.838 (0.66), 2.862 (0.87), 3.379 (1.55), 3.397 (2.36), 3.426 (2.10), 3.466 (1.78), 3.591 (4.09), 3.603 (6.71), 3.615 (3.86), 3.706 (1.18), 3.813 (2.10), 3.825 (16.00), 3.848 (1.68), 4.201 (0.92), 4.218 (1.94), 4.234 (1.97), 4.250 (0.92), 4.259 (0.66), 4.299 (0.97), 4.335 (0.84), 4.615 (1.70), 4.654 (1.57), 5.225 (1.23), 5.265 (1.52), 5.659 (0.81), 5.698 (0.71), 6.452 (0.73), 6.470 (0.76), 6.775 (1.70), 6.792 (1.89), 6.885 (2.31), 6.903 (2.57), 6.915 (0.97), 6.934 (1.63), 6.952 (0.94), 6.980 (1.73), 6.999 (2.33), 7.017 (1.42), 7.096 (1.10), 7.115 (1.99), 7.134 (1.02), 7.360 (2.18), 7.373 (2.26), 7.378 (1.97), 7.393 (3.28), 7.412 (2.62), 7.454 (3.46), 7.475 (1.97), 7.497 (0.47), 7.502 (0.76), 7.514 (2.12), 7.521 (2.96), 7.530 (4.46), 7.538 (3.25), 7.545 (2.26), 7.557 (0.79), 7.562 (0.42), 7.698 (2.02), 7.717 (1.89), 7.863 (1.99), 7.872 (1.05), 7.880 (1.47), 7.886 (1.70), 8.278 (1.76), 8.285 (1.47), 8.294 (0.81), 8.302 (1.57).

The title compound (124 mg) was separated into enantiomers by preparative chiral HPLC to give enantiomer 1 (50 mg, see Example 31) and enantiomer 2 (48 mg, see Example 32).

Preparative Chiral HPLC Method:

Instrument: Labomatic HD5000, Labocord-5000; Gilson GX-241, Labcol Vario 4000, column: Chiralpak ID 5μ 250×30 mm; Eluent A: hexane+0.1 Vol-% N-ethylethanamine (99%); Eluent B: ethanol; Gradient: 20-50% B in 20 min; Flow 40.0 mL/min; UV 254 nm

Analytical Chiral HPLC Method:

Instrument: Agilent HPLC 1260; column: Chiralpak ID 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 31

### (+)-4,5-Dimethyl-8-[2-(morpholin-4-yl)ethanesulfonyl]-17-{3-[(naphthalen-1-yl)oxy]propyl}-7,8,9,14-tetrahydro-5H-indolo[1,7-bc]pyrazolo[4,3-e][2,8]benzodiazacycloundecine-16-carboxylic acid-N-ethylethanamine Salt (Enantiomer 1)

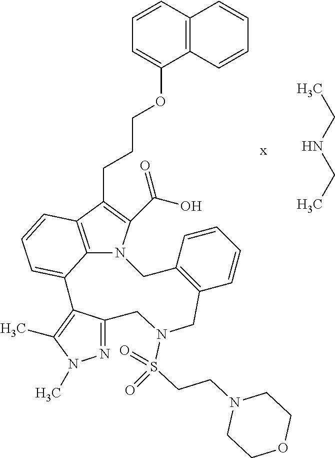

For the preparation of the racemic title compound see Example 30. Separation of enantiomers by preparative chiral HPLC (method see Example 30) gave the title compound (50 mg).

Analytical Chiral HPLC (method see Example 30): Rₜ=5.23 min.

LC-MS (Method 2): Rₜ=0.94 min, MS (ESIpos): m/z=748 [M+H]⁺

Specific Optical Rotation (Method O1): +73.8° (c=10 mg/mL, DMSO)

¹H-NMR (400 MHz, DMSO-d6) δ [ppm]: 0.798 (0.46), 0.815 (0.58), 0.822 (0.54), 0.905 (0.54), 1.131 (6.48), 1.149 (14.71), 1.167 (6.94), 1.232 (0.83), 1.259 (0.42), 1.670 (12.80), 2.296 (1.25), 2.314 (1.95), 2.318 (1.99), 2.322 (2.54), 2.327 (3.37), 2.332 (2.95), 2.518 (10.97), 2.523 (7.77), 2.539 (3.32), 2.660 (0.79), 2.664 (1.75), 2.669 (2.41), 2.673 (1.75), 2.678 (0.75), 2.829 (0.75), 2.863 (2.04), 2.881 (5.74), 2.900 (5.36), 2.917 (1.75), 3.384 (1.45), 3.401 (0.91), 3.416 (0.79), 3.430 (1.87), 3.469 (1.75), 3.595 (3.62), 3.607 (6.15), 3.618 (3.57), 3.748 (0.96), 3.822 (16.00), 3.852 (1.58), 4.188 (0.75), 4.196 (0.79), 4.213 (1.33), 4.230 (1.00), 4.247 (1.29), 4.263 (0.75), 4.271 (0.75), 4.310 (0.87), 4.345 (0.79), 4.596 (1.58), 4.635 (1.45), 5.114 (0.66), 5.153 (0.75), 6.653 (1.29), 6.846 (0.66), 6.865 (1.16), 6.882 (2.87), 6.899 (3.03), 6.915 (1.33), 6.934 (0.71), 7.051 (0.91), 7.069 (1.66), 7.088 (0.83), 7.335 (1.95), 7.352 (1.70), 7.364 (1.91), 7.385 (3.28), 7.404 (2.70), 7.444 (3.37), 7.464 (1.87), 7.490 (0.42), 7.495 (0.71), 7.507 (2.24), 7.512 (3.62), 7.522 (4.45), 7.531 (4.03), 7.537 (2.37), 7.548 (0.79), 7.554 (0.42), 7.584 (0.96), 7.603 (0.87), 7.856 (1.91), 7.860 (1.41), 7.866 (0.96), 7.872 (1.25), 7.874 (1.33), 7.879 (1.66), 8.271 (1.70), 8.278 (1.29), 8.286 (0.79), 8.295 (1.58).

### Example 32

### (−)-4,5-Dimethyl-8-[2-(morpholin-4-yl)ethanesulfonyl]-17-{3-[(naphthalen-1-yl)oxy]propyl}-7,8,9,14-tetrahydro-5H-indolo[1,7-bc]pyrazolo[4,3-e][2,8]benzodiazacycloundecine-16-carboxylic acid-N-ethylethanamine Salt (Enantiomer 2)

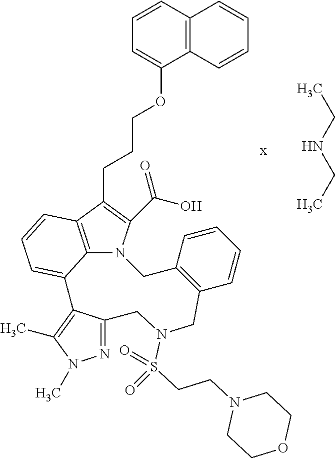

For the preparation of the racemic title compound see Example 30. Separation of enantiomers by preparative chiral HPLC (method see Example 30) gave the title compound (48 mg).

Analytical Chiral HPLC (method see Example 30): Rₜ=7.56 min.

LC-MS (Method 2): Rₜ=0.94 min, MS (ESIpos): m/z=748 [M+H]⁺

Specific Optical Rotation (Method O1): −69.0° (c=10 mg/mL, DMSO)

¹H-NMR (400 MHz, DMSO-d6) δ [ppm]: 0.795 (0.48), 0.798 (0.44), 0.815 (0.57), 0.822 (0.53), 0.905 (0.48), 1.006 (0.44), 1.084 (0.48), 1.128 (5.49), 1.146 (12.48), 1.164 (5.63), 1.205 (0.66), 1.232 (1.23), 1.259 (0.79), 1.669 (12.26), 2.297 (1.27), 2.317 (2.15), 2.322 (2.64), 2.327 (3.43), 2.332 (3.08), 2.518 (10.81), 2.523 (7.82), 2.539 (1.19), 2.660 (0.84), 2.664 (1.80), 2.669 (2.51), 2.673 (1.80), 2.678 (0.79), 2.831 (0.70), 2.855 (1.93), 2.874 (4.92), 2.891 (4.88), 2.910 (1.80), 3.382 (1.41), 3.397 (0.88), 3.414 (0.79), 3.430 (1.89), 3.470 (1.71), 3.596 (3.56), 3.608 (5.98), 3.619 (3.47), 3.754 (0.97), 3.822 (16.00), 3.853 (1.54), 4.188 (0.75), 4.195 (0.75), 4.212 (1.27), 4.230 (0.79), 4.250 (1.32), 4.266 (0.75), 4.273 (0.79), 4.290 (0.40), 4.312 (0.88), 4.347 (0.75), 4.591 (1.54), 4.631 (1.45), 5.094 (0.75), 5.134 (0.84), 6.613 (0.84), 6.630 (0.92), 6.684 (0.57), 6.834 (0.70), 6.852 (1.19), 6.882 (3.16), 6.901 (3.74), 6.920 (0.88), 7.042 (0.92), 7.062 (1.71), 7.081 (0.88), 7.331 (1.93), 7.348 (1.67), 7.364 (1.89), 7.384 (3.30), 7.403 (2.77), 7.443 (3.34), 7.463 (1.89), 7.489 (0.40), 7.494 (0.75), 7.506 (2.24), 7.511 (3.74), 7.521 (4.48), 7.530 (4.18), 7.535 (2.37), 7.547 (0.97), 7.553 (0.66), 7.563 (1.14), 7.582 (1.05), 7.855 (1.93), 7.859 (1.36), 7.866 (0.97), 7.871 (1.23), 7.873 (1.32), 7.879 (1.67), 8.269 (1.67), 8.277 (1.27), 8.285 (0.79), 8.294 (1.54).

### Example 33

### (rac)-3-Chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic Acid

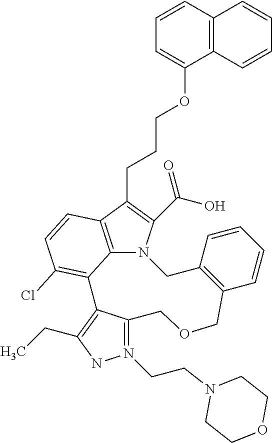

To a solution of (rac)-ethyl 3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylate (see intermediate 41, 50.0 mg, 66.9 μmol) in a mixture of THF (2 mL) and ethanol (1 mL) was added an aqueous solution of lithium hydroxide (1 mL, 1.0 M, 1 mmol). The resulting mixture was stirred at 50° C. for 17 hours, followed by stirring for one day at ambient temperature. After removal of all volatiles, the residue was subjected to flash chromatography (Biotage SNAP cartridge silica, dichloromethane/ethanol gradient, 0%->25% ethanol) to give the title compound (41 mg).

LC-MS (Method 2): Rt=0.95 min, MS (ESIpos): m/z=720 [M+H]⁺

¹H-NMR (400 MHz, DMSO-d6) δ [ppm]: 0.798 (0.60), 0.815 (0.66), 0.822 (0.73), 0.840 (7.54), 0.859 (16.00), 0.877 (7.47), 0.905 (0.73), 1.035 (2.18), 1.053 (4.89), 1.071 (2.31), 1.232 (2.58), 1.907 (0.73), 1.988 (0.40), 2.006 (0.46), 2.062 (1.65), 2.081 (4.89), 2.100 (4.76), 2.119 (1.72), 2.159 (3.77), 2.174 (5.49), 2.188 (3.44), 2.336 (1.26), 2.378 (2.12), 2.389 (1.92), 2.405 (1.79), 2.518 (15.07), 2.523 (10.64), 2.539 (1.12), 2.556 (0.79), 2.570 (1.32), 2.586 (1.39), 2.601 (1.79), 2.615 (0.86), 2.690 (1.92), 2.708 (1.19), 2.722 (1.26), 2.741 (0.53), 3.160 (0.40), 3.179 (0.86), 3.195 (1.19), 3.213 (1.85), 3.233 (1.59), 3.253 (1.92), 3.271 (1.26), 3.287 (1.26), 3.406 (6.21), 3.417 (10.91), 3.429 (6.02), 3.452 (0.79), 3.874 (3.31), 3.908 (3.31), 3.998 (1.39), 4.026 (2.38), 4.086 (4.23), 4.095 (1.85), 4.113 (5.02), 4.130 (3.11), 4.146 (1.39), 4.154 (0.99), 4.170 (0.40), 4.276 (2.05), 4.290 (4.03), 4.306 (2.38), 4.355 (0.46), 4.753 (3.17), 4.787 (3.04), 5.184 (1.32), 5.222 (1.85), 5.380 (0.79), 5.423 (0.60), 6.807 (3.64), 6.825 (3.90), 6.921 (2.38), 6.941 (2.84), 7.110 (1.72), 7.123 (1.19), 7.130 (1.26), 7.143 (1.12), 7.164 (3.64), 7.173 (5.82), 7.179 (8.53), 7.191 (1.65), 7.200 (2.84), 7.350 (2.71), 7.371 (5.02), 7.390 (3.97), 7.441 (5.22), 7.462 (3.17), 7.471 (1.26), 7.475 (1.39), 7.488 (2.98), 7.492 (2.78), 7.506 (5.16), 7.511 (5.49), 7.526 (2.91), 7.530 (3.24), 7.543 (1.45), 7.547 (1.12), 7.723 (2.18), 7.744 (1.98), 7.850 (3.24), 7.868 (3.37), 7.873 (2.78), 8.198 (2.78), 8.202 (2.91), 8.221 (2.71), 13.508 (0.40).

The title compound (36 mg) was separated into enantiomers by preparative chiral HPLC to give enantiomer 1 (14 mg, see Example 34) and enantiomer 2 (14 mg, see Example 35).

Preparative Chiral HPLC Method:

Instrument: Labomatic HD5000, Labocord-5000; Gilson GX-241, Labcol Vario 4000, column: Chiralpak IG 5μ 250×30 mm; Eluent A: tert-butyl methyl ether+0.1 Vol-% N-ethylethanamine (99%); Eluent B: ethanol; isocratic 90% A+10% B; flow 40.0 mL/min; UV 254 nm

Analytical Chiral HPLC Method

Instrument: Agilent HPLC 1260; column: Chiralpak IG 3μ 100×4.6 mm; Eluent A: tert-butyl methyl ether+0.1 Vol-% N-ethylethanamine (99%); Eluent B: ethanol; Gradient: 20-50% B in 7 min; Flow 1.4 mL/min; Temperature: 25° C.; DAD 254 nm

### Example 34

### 3-Chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid-N-ethylethanamine Salt (Enantiomer 1)

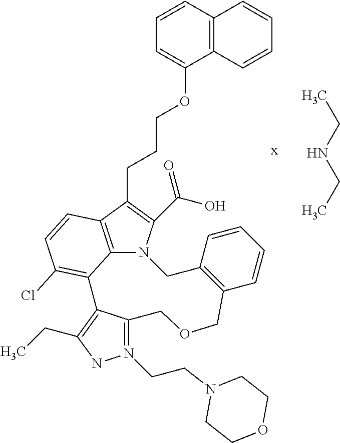

For the preparation of the racemic title compound see Example 33. Separation of enantiomers by preparative chiral HPLC (method see Example 33) gave the title compound (14 mg).

Analytical Chiral HPLC (method see Example 33): Rₜ=1.55 min.

LC-MS (Method 2): Rt=0.97 min, MS (ESIpos): m/z=720 [M+H]⁺

¹H-NMR (400 MHz, DMSO-d6) δ [ppm]: 0.803 (3.55), 0.822 (7.63), 0.841 (3.81), 0.852 (0.85), 0.860 (0.69), 0.967 (0.42), 1.130 (7.26), 1.149 (16.00), 1.166 (7.42), 1.232 (2.01), 1.256 (0.69), 1.986 (0.42), 2.007 (0.79), 2.030 (1.48), 2.040 (1.17), 2.048 (1.38), 2.066 (0.58), 2.182 (1.48), 2.199 (2.86), 2.216 (2.38), 2.230 (1.80), 2.332 (2.23), 2.336 (0.95), 2.404 (1.22), 2.433 (1.01), 2.518 (11.76), 2.523 (8.37), 2.539 (1.17), 2.611 (0.64), 2.628 (0.74), 2.644 (0.95), 2.673 (2.28), 2.678 (1.01), 2.708 (0.48), 2.727 (1.06), 2.744 (0.64), 2.758 (0.64), 2.864 (1.75), 2.883 (5.46), 2.900 (5.19), 2.919 (1.64), 3.200 (0.85), 3.219 (1.64), 3.230 (1.59), 3.248 (0.85), 3.437 (3.44), 3.449 (5.93), 3.459 (3.34), 3.874 (2.17), 3.909 (2.23), 4.094 (1.06), 4.117 (4.29), 4.133 (1.54), 4.150 (1.43), 4.167 (0.74), 4.174 (0.74), 4.292 (1.54), 4.309 (2.60), 4.324 (1.43), 4.728 (1.80), 4.763 (1.75), 5.059 (0.48), 5.100 (0.53), 6.813 (2.17), 6.831 (2.28), 6.962 (0.85), 6.980 (1.75), 7.001 (0.69), 7.018 (0.90), 7.060 (0.74), 7.092 (0.95), 7.111 (1.38), 7.131 (0.79), 7.170 (2.44), 7.188 (1.38), 7.345 (1.70), 7.366 (3.13), 7.385 (2.44), 7.434 (3.13), 7.455 (1.85), 7.473 (0.58), 7.477 (0.79), 7.490 (1.85), 7.494 (1.64), 7.503 (2.07), 7.509 (3.50), 7.514 (2.01), 7.523 (1.85), 7.527 (2.07), 7.540 (0.90), 7.544 (0.64), 7.587 (0.58), 7.847 (1.91), 7.853 (1.11), 7.865 (2.01), 7.870 (1.59), 8.215 (1.59), 8.219 (1.59), 8.239 (1.48).

### Example 35

### 3-Chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid-N-ethylethanamine Salt (Enantiomer 2)

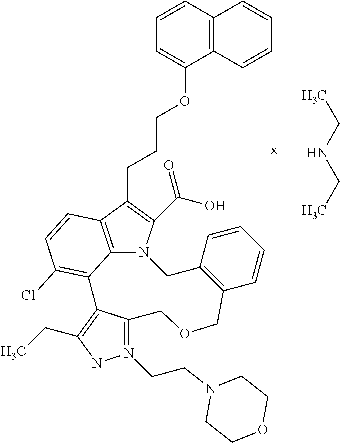

For the preparation of the racemic title compound see Example 33. Separation of enantiomers by preparative chiral HPLC (method see Example 33) gave the title compound (14 mg).

Analytical Chiral HPLC (method see Example 33): Rₜ=3.17 min.

LC-MS (Method 2): Rt=0.95 min, MS (ESIpos): m/z=720 [M+H]⁺

¹H-NMR (400 MHz, DMSO-d6) δ [ppm]: 0.808 (3.68), 0.827 (7.96), 0.846 (3.98), 0.860 (0.91), 0.967 (0.48), 1.132 (6.96), 1.150 (16.00), 1.169 (7.05), 1.232 (1.77), 1.256 (0.91), 1.296 (0.56), 2.015 (0.65), 2.036 (1.60), 2.055 (1.47), 2.179 (1.69), 2.195 (2.85), 2.211 (2.25), 2.332 (1.82), 2.336 (0.78), 2.401 (1.17), 2.428 (0.99), 2.518 (9.60), 2.523 (6.75), 2.539 (0.74), 2.605 (0.65), 2.622 (0.74), 2.637 (0.95), 2.651 (0.48), 2.678 (0.82), 2.703 (0.48), 2.722 (1.04), 2.739 (0.65), 2.753 (0.65), 2.869 (1.69), 2.887 (5.15), 2.905 (5.10), 2.923 (1.56), 3.200 (0.74), 3.218 (1.69), 3.234 (1.60), 3.250 (0.82), 3.266 (0.56), 3.433 (3.37), 3.444 (5.88), 3.455 (3.33), 3.823 (0.56), 3.874 (2.12), 3.909 (2.12), 4.077 (0.39), 4.108 (3.55), 4.131 (1.38), 4.147 (1.38), 4.163 (0.74), 4.171 (0.69), 4.289 (1.47), 4.306 (2.51), 4.322 (1.43), 4.731 (1.82), 4.766 (1.73), 5.079 (0.48), 5.115 (0.52), 6.812 (2.08), 6.829 (2.25), 6.956 (0.91), 6.977 (1.56), 7.013 (0.61), 7.031 (0.86), 7.048 (0.52), 7.077 (0.74), 7.099 (1.34), 7.118 (1.47), 7.137 (0.86), 7.169 (2.29), 7.188 (1.30), 7.345 (1.73), 7.366 (3.16), 7.385 (2.46), 7.435 (3.11), 7.456 (1.86), 7.472 (0.61), 7.476 (0.78), 7.489 (1.82), 7.494 (1.64), 7.503 (2.03), 7.509 (3.16), 7.513 (2.08), 7.523 (1.82), 7.527 (2.12), 7.540 (0.91), 7.544 (0.65), 7.606 (0.61), 7.847 (1.86), 7.853 (1.12), 7.866 (2.12), 7.870 (1.64), 8.212 (1.56), 8.217 (1.56), 8.236 (1.51).

### Example 36

### (rac)-3-Chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic Acid

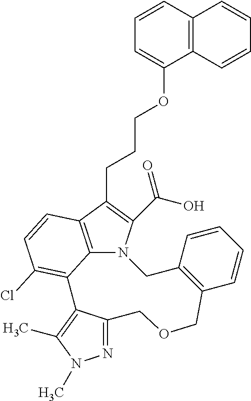 [SMILES: Cc1c2c(nn1C)COCc1ccccc1Cn1c(C(=O)O)c(CCCOc3cccc4ccccc34)c3ccc(Cl)c-2c31]

(rac)-Ethyl 3-chloro-4,5-dimethyl-17-[3-(naphthalen-1-yloxy)propyl]-5,7,9,14-tetrahydroindolo-[7,1-fg]pyrazolo[3,4-d][2,8]benzoxazacycloundecine-16-carboxylate (see intermediate 44, 400 mg, 631 μmol) was dissolved in a mixture of 9 mL of THF and 3 mL of ethanol, and aqueous lithium hydroxide solution (1.3 mL, 1.0 M, 1.3 mmol) was added. The mixture was stirred at 70° C. for 2 days, and at 80° C. for 6 hours. After concentration, water and aqueous saturated citric acid solution were added until an acidic pH value was reached. The mixture was extracted with THF. The combined organic layers were dried over sodium sulfate, filtered and concentrated under reduced pressure to give the title compound (354 mg, 98% yield).

LC-MS (Method 1): Rₜ=1.53 min, MS (ESIpos): m/z=606 [M+H]⁺

¹H-NMR (400 MHz, DMSO-d6) δ [ppm]: 0.000 (1.80), 0.835 (2.23), 1.003 (1.65), 1.020 (3.50), 1.038 (1.85), 1.117 (0.41), 1.134 (0.94), 1.200 (0.69), 1.217 (0.68), 1.745 (14.01), 2.098 (1.03), 2.115 (1.62), 2.132 (1.16), 2.182 (0.79), 2.485 (3.24), 2.490 (2.03), 3.123 (0.79), 3.141 (1.56), 3.152 (1.48), 3.170 (0.86), 3.184 (0.46), 3.379 (1.03), 3.397 (1.21), 3.414 (1.04), 3.431 (0.47), 3.802 (16.00), 4.022 (1.40), 4.030 (0.98), 4.037 (0.94), 4.048 (2.48), 4.070 (0.52), 4.110 (2.17), 4.122 (2.61), 4.135 (1.93), 4.142 (1.06), 4.155 (2.35), 4.344 (2.44), 4.377 (2.00), 4.906 (1.00), 4.942 (1.13), 5.700 (0.73), 5.737 (0.69), 6.776 (1.80), 6.794 (1.93), 6.988 (2.35), 6.999 (0.57), 7.009 (3.77), 7.016 (1.74), 7.020 (1.65), 7.027 (2.26), 7.033 (3.51), 7.042 (2.85), 7.048 (1.38), 7.055 (0.96), 7.314 (1.37), 7.334 (2.55), 7.353 (3.03), 7.363 (1.21), 7.375 (1.00), 7.392 (2.71), 7.413 (1.56), 7.437 (2.28), 7.449 (1.82), 7.453 (1.86), 7.461 (2.68), 7.467 (3.45), 7.474 (1.84), 7.481 (1.59), 7.485 (1.84), 7.498 (0.75), 7.502 (0.49), 7.807 (1.56), 7.814 (0.94), 7.825 (1.65), 7.830 (1.35), 8.170 (1.38), 8.175 (1.36), 8.192 (1.28), 8.194 (1.31)

The title compound (344 mg) was separated into enantiomers by preparative chiral HPLC to give enantiomer 1 (156 mg, see Example 37) and enantiomer 2 (209 mg, see Example 38).

Preparative Chiral HPLC Method:

Instrument: Labomatic HD5000, Labocord-5000; Gilson GX-241, Labcol Vario 4000, column: Chiralpak IE 5μ 250×30 mm; Eluent A: hexane+0.1 Vol-% trifluoroacetic acid; Eluent B: 2-propanol; isocratic 70% A+30% B; Flow 50.0 mL/min; UV 254 nm

Analytical Chiral HPLC Method:

Instrument: Agilent HPLC 1260; column: Chiralpak IE 3μ 100×4.6 mm; Eluent A: hexane+0.1 Vol-% trifluoroacetic acid; Eluent B: 2-propanol; isocratic 70% A+30% B; Flow 1.4 mL/min; Temperature: 25° C.; DAD 254 nm

### Example 37

### (−)-3-Chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic Acid (Enantiomer 1)

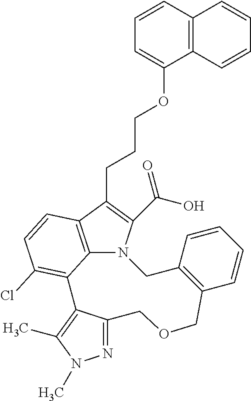 [SMILES: Cc1c2c(nn1C)COCc1ccccc1Cn1c(C(=O)O)c(CCCOc3cccc4ccccc34)c3ccc(Cl)c-2c31]

For the preparation of the racemic title compound see Example 36. Separation of enantiomers by preparative chiral HPLC (method see Example 36) gave the title compound (156 mg).

Analytical Chiral HPLC (method see Example 36): Rₜ=2.33 min.

LC-MS (Method 1): Rₜ=1.52 min; MS (ESIpos): m/z=606 [M+H]⁺

Specific Optical Rotation (Method O1): −62.2° (c=10 mg/mL, CHCl₃)

¹H-NMR (400 MHz, DMSO-d6) δ [ppm]: 1.135 (1.06), 1.153 (2.09), 1.172 (1.09), 1.232 (0.52), 1.864 (13.28), 2.095 (1.12), 2.111 (1.63), 2.128 (1.12), 2.322 (1.18), 2.326 (1.61), 2.331 (1.15), 2.522 (4.42), 2.664 (1.18), 2.668 (1.61), 2.919 (0.43), 2.933 (0.43), 3.128 (0.57), 3.143 (0.60), 3.161 (0.97), 3.181 (0.46), 3.204 (0.46), 3.222 (0.97), 3.240 (0.66), 3.255 (0.57), 3.837 (16.00), 3.941 (2.70), 3.966 (2.90), 4.070 (2.75), 4.078 (1.86), 4.095 (2.44), 4.103 (1.84), 4.113 (2.47), 4.127 (0.89), 4.145 (2.27), 4.407 (2.21), 4.439 (1.84), 5.215 (0.92), 5.252 (2.21), 5.289 (2.06), 5.326 (0.92), 5.759 (0.57), 6.790 (1.81), 6.808 (1.92), 7.097 (1.20), 7.113 (1.92), 7.140 (0.60), 7.151 (1.32), 7.163 (1.49), 7.187 (4.93), 7.196 (1.95), 7.228 (3.10), 7.249 (3.30), 7.353 (1.20), 7.374 (2.35), 7.393 (1.78), 7.440 (2.55), 7.460 (1.49), 7.472 (0.60), 7.486 (1.38), 7.505 (2.35), 7.509 (2.47), 7.524 (1.35), 7.528 (1.41), 7.541 (0.63), 7.717 (3.01), 7.739 (2.67), 7.847 (1.63), 7.866 (1.55), 8.181 (1.55), 8.201 (1.38).

### Example 38

### (+)-3-Chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic Acid (Enantiomer 2)

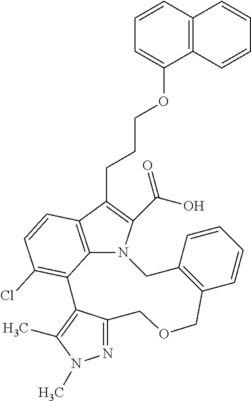 [SMILES: Cc1c2c(nn1C)COCc1ccccc1Cn1c(C(=O)O)c(CCCOc3cccc4ccccc34)c3ccc(Cl)c-2c31]

For the preparation of the racemic title compound see Example 36. Separation of enantiomers by preparative chiral HPLC (method see Example 36) gave the title compound (209 mg).

Analytical Chiral HPLC (method see Example 36): Rₜ=2.96 min.

LC-MS (Method 1): Rₜ=1.52 min, MS (ESIpos): m/z=606 [M+H]⁺

Specific Optical Rotation (Method O1): +73.3° (c=10 mg/mL, CHCl₃)

¹H-NMR (400 MHz, DMSO-d6) δ [ppm]: 1.026 (0.55), 1.042 (0.59), 1.135 (1.47), 1.154 (2.86), 1.172 (1.43), 1.234 (0.97), 1.864 (16.00), 2.095 (1.31), 2.111 (1.89), 2.128 (1.31), 2.331 (1.68), 2.518 (10.11), 2.522 (6.32), 2.673 (1.73), 2.902 (0.42), 2.920 (0.55), 2.933 (0.55), 3.128 (0.67), 3.143 (0.72), 3.162 (1.14), 3.181 (0.55), 3.204 (0.55), 3.222 (1.14), 3.240 (0.72), 3.255 (0.76), 3.880 (3.20), 3.941 (3.96), 3.966 (4.08), 4.070 (3.45), 4.078 (2.40), 4.096 (2.99), 4.103 (2.32), 4.113 (3.07), 4.145 (2.78), 4.407 (2.65), 4.439 (2.27), 5.215 (1.05), 5.252 (2.61), 5.289 (2.44), 5.326 (1.09), 6.790 (2.11), 6.808 (2.23), 7.097 (1.39), 7.114 (2.27), 7.140 (0.76), 7.151 (1.52), 7.163 (1.73), 7.188 (5.77), 7.196 (2.32), 7.228 (4.04), 7.249 (4.25), 7.353 (1.47), 7.374 (2.78), 7.393 (2.15), 7.440 (2.99), 7.460 (1.81), 7.473 (0.76), 7.486 (1.64), 7.505 (2.82), 7.509 (2.99), 7.524 (1.73), 7.528 (1.85), 7.541 (0.84), 7.717 (3.79), 7.739 (3.33), 7.847 (1.89), 7.866 (1.89), 8.181 (1.77), 8.201 (1.64).

### Example 39

### (rac)-3-Chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic Acid

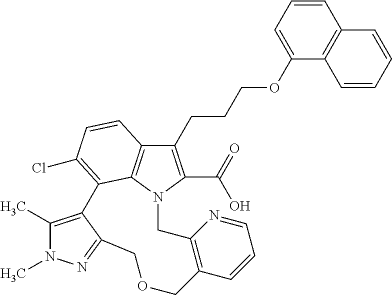 [SMILES: Cc1c2c(nn1C)COCc1cccnc1Cn1c(C(=O)O)c(CCCOc3cccc4ccccc34)c3ccc(Cl)c-2c31]

(rac)-Ethyl 3-chloro-4,5-dimethyl-17-[3-(naphthalen-1-yloxy)propyl]-5,7,9,14-tetrahydropyrazolo[4′,3′:9,10]pyrido[3′,2′:3,4][1,6]oxazacycloundecino[8,7,6-hi]indole-16-carboxylate (see intermediate 45, 86.0 mg, 135 μmol) was dissolved in a mixture of 1 mL of THF and 100 μL of ethanol, and aqueous lithium hydroxide solution (270 μL, 1.0 M, 270 μmol) was added. The reaction mixture was stirred at 70° C. for 22 hours in a sealed tube. Aqueous lithium hydroxide solution (270 μL, 1.0 M, 270 μmol) was added, and stirring was continued at 70° C. for 2 days in a sealed tube. Aqueous lithium hydroxide solution (135 μL, 1.0 M, 135 μmol) was added, and stirring was continued at 70° C. for 5 hours and additional 3 days at rt in a sealed tube. The reaction mixture was diluted with water and acidified using aqueous, saturated citric acid solution until a pH value of 3-4 was reached. The mixture was extracted with ethyl acetate and the combined organic layers were concentrated under reduced pressure. The crude material was purified by flash chromatography using a silica gel (gradient dichloromethane/ethanol). The product was dissolved in ethyl acetate and ethanol and washed with water. The aqueous phase was extracted with ethyl acetate and the combined organic phases were dried with a water resistant filter and concentrated to obtain the title compound (36.8 mg, 43% yield).

LC-MS (Method 1): Rₜ=1.47 min; MS (ESIneg): m/z=607 [M+H]⁺

¹H-NMR (400 MHz, DMSO-d6) δ [ppm]: 1.149 (2.19), 1.167 (4.36), 1.185 (2.15), 1.227 (0.83), 1.814 (16.00), 1.983 (8.10), 2.135 (1.10), 2.152 (1.63), 2.169 (1.22), 2.186 (0.49), 2.326 (0.73), 2.518 (3.12), 2.668 (0.73), 3.206 (1.07), 3.223 (1.71), 3.235 (1.85), 3.252 (1.12), 3.268 (0.83), 3.863 (14.58), 3.995 (0.66), 4.012 (1.95), 4.030 (1.92), 4.048 (0.68), 4.076 (0.93), 4.084 (0.93), 4.095 (2.25), 4.120 (2.24), 4.147 (0.59), 4.163 (1.27), 4.180 (0.76), 4.187 (0.90), 4.289 (1.93), 4.314 (1.56), 4.348 (1.97), 4.380 (2.47), 4.518 (1.59), 4.550 (1.17), 5.075 (1.49), 5.112 (1.90), 5.414 (1.08), 5.451 (0.92), 6.817 (2.08), 6.835 (2.22), 7.056 (1.53), 7.067 (1.58), 7.075 (1.64), 7.087 (1.59), 7.140 (3.17), 7.161 (3.29), 7.360 (1.34), 7.380 (2.73), 7.399 (2.12), 7.437 (3.08), 7.457 (1.66), 7.477 (0.44), 7.481 (0.63), 7.494 (1.71), 7.499 (1.81), 7.502 (2.12), 7.510 (3.59), 7.518 (2.47), 7.526 (3.44), 7.544 (2.19), 7.547 (1.81), 7.585 (3.14), 7.606 (2.85), 7.845 (1.76), 7.853 (0.98), 7.862 (1.53), 7.868 (1.51), 8.208 (1.61), 8.215 (1.46), 8.233 (1.49), 8.279 (1.95), 8.284 (2.00), 8.291 (1.97), 8.295 (1.83).

The title compound (25 mg) was separated into enantiomers by preparative chiral HPLC to give enantiomer 1 (7 mg, see Example 40) and enantiomer 2 (8 mg, see Example 41).

Preparative Chiral HPLC Method:

Instrument: Labomatic HD5000, Labocord-5000; Gilson GX-241, Labcol Vario 4000, column: Chiralpak ID 5μ 250×30 mm; Eluent A: hexane+0.1 Vol-% N-ethylethanamine (99%); Eluent B: ethanol; isocratic 50% A+50% B; Flow 50.0 mL/min; UV 254 nm

Analytical Chiral HPLC Method:

Instrument: Agilent HPLC 1260; column: Chiralpak ID 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 40

### 3-Chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid-N-ethylethanamine Salt (Enantiomer 1)

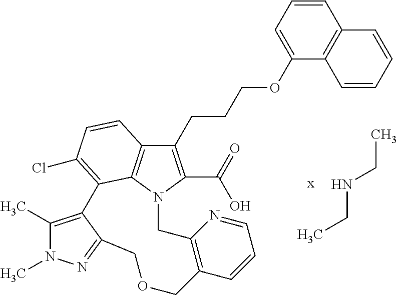

For the preparation of the racemic title compound see Example 39. Separation of enantiomers by preparative chiral HPLC (method see Example 39) gave the title compound (7 mg).

Analytical Chiral HPLC (method see Example 39): Rₜ=3.77 min.

LC-MS (Method 1): Rₜ=1.47 min, MS (ESIpos): m/z=607 [M+H]⁺

¹H-NMR (400 MHz, DMSO-d6) δ [ppm]: 1.111 (7.62), 1.129 (16.00), 1.147 (7.89), 1.820 (10.05), 2.141 (0.53), 2.157 (0.82), 2.170 (0.80), 2.186 (0.59), 2.331 (0.86), 2.518 (5.42), 2.522 (3.29), 2.818 (2.13), 2.836 (6.52), 2.855 (6.23), 2.872 (2.00), 3.210 (0.96), 3.228 (1.59), 3.247 (1.88), 3.846 (8.99), 4.091 (0.55), 4.098 (0.51), 4.118 (1.31), 4.144 (1.29), 4.178 (0.80), 4.195 (0.47), 4.202 (0.51), 4.278 (1.21), 4.303 (0.94), 4.339 (1.10), 4.370 (1.63), 4.450 (0.92), 4.482 (0.55), 5.050 (0.92), 5.088 (1.10), 5.539 (0.43), 6.822 (1.27), 6.841 (1.37), 7.046 (0.96), 7.058 (1.00), 7.065 (1.00), 7.077 (1.00), 7.113 (1.92), 7.134 (2.00), 7.360 (0.88), 7.380 (1.70), 7.399 (1.35), 7.438 (1.88), 7.459 (1.04), 7.482 (0.41), 7.495 (1.06), 7.499 (1.02), 7.505 (1.19), 7.512 (2.37), 7.519 (2.31), 7.524 (2.19), 7.528 (1.33), 7.538 (1.25), 7.542 (1.39), 7.559 (1.90), 7.580 (1.68), 7.848 (1.10), 7.856 (0.63), 7.866 (1.02), 7.871 (0.92), 8.208 (0.96), 8.214 (0.92), 8.232 (0.90), 8.260 (1.19), 8.264 (1.19), 8.271 (1.19), 8.275 (1.06).

### Example 41

### 3-Chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid-N-ethylethanamine Salt (Enantiomer 2)

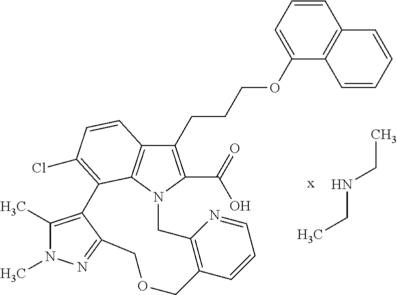

For the preparation of the racemic title compound see Example 39. Separation of enantiomers by preparative chiral HPLC (method see Example 39) gave the title compound (8 mg).

Analytical Chiral HPLC (method see Example 39): Rₜ=6.08 min.

LC-MS (Method 1): Rₜ=1.47 min, MS (ESIpos): m/z=607 [M+H]⁺

¹H-NMR (400 MHz, DMSO-d6) δ [ppm]: 1.090 (5.20), 1.108 (10.95), 1.127 (5.40), 1.229 (0.47), 1.822 (16.00), 2.128 (0.41), 2.144 (0.89), 2.161 (1.46), 2.178 (1.44), 2.195 (0.93), 2.211 (0.43), 2.331 (0.82), 2.518 (5.20), 2.522 (3.11), 2.669 (1.15), 2.673 (0.85), 2.744 (1.36), 2.761 (3.88), 2.780 (3.83), 2.798 (1.26), 3.173 (0.70), 3.188 (0.99), 3.206 (1.55), 3.223 (2.02), 3.240 (2.08), 3.254 (2.85), 3.821 (13.73), 4.101 (0.85), 4.107 (0.80), 4.124 (1.24), 4.143 (1.79), 4.170 (2.58), 4.187 (1.30), 4.204 (0.78), 4.210 (0.85), 4.262 (2.04), 4.288 (1.44), 4.320 (1.09), 4.352 (2.89), 4.375 (1.51), 4.408 (0.52), 5.019 (1.46), 5.056 (1.73), 5.644 (0.60), 5.682 (0.54), 6.825 (2.12), 6.843 (2.25), 7.033 (1.50), 7.044 (1.55), 7.052 (1.59), 7.063 (1.55), 7.083 (2.83), 7.104 (2.99), 7.354 (1.38), 7.374 (2.74), 7.393 (2.12), 7.433 (3.07), 7.454 (1.69), 7.474 (0.50), 7.478 (0.70), 7.491 (1.79), 7.495 (1.69), 7.502 (2.02), 7.509 (5.28), 7.514 (3.40), 7.521 (2.17), 7.530 (4.54), 7.542 (0.76), 7.551 (2.56), 7.845 (1.81), 7.853 (1.01), 7.864 (1.65), 7.869 (1.50), 8.206 (1.57), 8.212 (1.53), 8.230 (1.65), 8.238 (1.92), 8.242 (1.92), 8.249 (1.84), 8.253 (1.65).

### Example 42

### (rac)-3-Chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic Acid

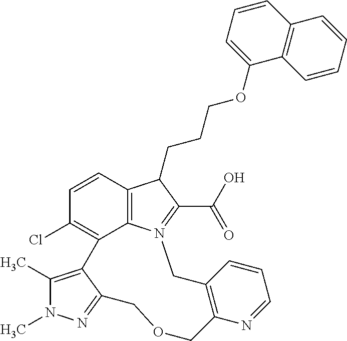

(rac)-Ethyl 3-chloro-4,5-dimethyl-17-[3-(naphthalen-1-yloxy)propyl]-5,7,9,14-tetrahydropyrazolo[4′,3′:9,10]pyrido[2′,3′:3,4][1,6]oxazacycloundecino[8,7,6-hi]indole-16-carboxylate (see intermediate 46, 59.0 mg, 92.9 μmol) was dissolved in a mixture of 1 mL of THF and 100 μL of ethanol and aqueous lithium hydroxide solution (190 μL, 1.0 M, 190 μmol) was added. The mixture was stirred at 70° C. for 23 hours in a sealed tube. The reaction mixture was diluted with water and acidified using aqueous, saturated citric acid solution until a pH value of 3-4 was reached. The mixture was extracted with ethyl acetate and the combined organic layers were concentrated under reduced pressure. The crude material was purified by flash chromatography using silica gel (gradient dichloromethane/ethanol). The product was dissolved in ethyl acetate and ethanol and washed with water. The aqueous phase was extracted with ethyl acetate and the combined organic phases were dried with a water resistant filter and concentrated to obtain the title compound (36.8 mg, 62% yield).

LC-MS (Method 1): Rₜ=1.39 min, MS (ESIpos): m/z=607 [M+H]⁺

¹H-NMR (600 MHz, DMSO-d6) δ [ppm]: −0.100 (0.45), −0.006 (3.34), 0.005 (3.40), 0.097 (0.45), 1.166 (4.29), 1.178 (8.56), 1.190 (4.25), 1.234 (0.60), 1.884 (14.14), 1.910 (2.80), 1.990 (16.00), 2.100 (0.62), 2.111 (1.81), 2.123 (2.80), 2.134 (1.92), 2.145 (0.68), 2.386 (0.91), 2.389 (1.26), 2.392 (0.95), 2.520 (3.32), 2.524 (3.22), 2.526 (2.58), 2.614 (0.93), 2.618 (1.24), 2.620 (0.91), 3.152 (0.47), 3.165 (0.97), 3.174 (0.99), 3.187 (1.65), 3.200 (0.70), 3.232 (0.74), 3.245 (1.65), 3.257 (0.99), 3.267 (1.07), 3.279 (0.52), 3.822 (15.15), 4.011 (1.34), 4.023 (3.79), 4.035 (4.16), 4.040 (1.44), 4.047 (1.71), 4.057 (1.69), 4.064 (0.72), 4.075 (1.15), 4.080 (1.18), 4.085 (0.87), 4.091 (2.06), 4.104 (1.24), 4.114 (2.06), 4.119 (0.87), 4.125 (1.15), 4.130 (1.09), 4.140 (0.47), 4.189 (2.52), 4.211 (2.78), 4.235 (3.09), 4.252 (2.80), 4.442 (3.94), 4.464 (3.46), 5.257 (2.06), 5.283 (3.26), 5.368 (2.35), 5.393 (1.71), 6.803 (3.22), 6.816 (3.32), 7.233 (2.41), 7.241 (2.43), 7.247 (7.28), 7.254 (2.68), 7.261 (5.51), 7.364 (2.19), 7.377 (4.08), 7.391 (2.97), 7.441 (4.08), 7.455 (2.72), 7.480 (1.05), 7.482 (1.18), 7.491 (2.12), 7.493 (2.45), 7.504 (2.33), 7.507 (2.29), 7.510 (2.37), 7.513 (2.39), 7.524 (2.64), 7.525 (2.56), 7.534 (1.34), 7.537 (1.18), 7.555 (2.39), 7.558 (2.45), 7.569 (2.23), 7.571 (2.16), 7.738 (5.20), 7.752 (4.74), 7.849 (2.74), 7.861 (2.64), 8.193 (2.58), 8.206 (2.41), 8.313 (2.33), 8.335 (2.80), 8.337 (2.87), 8.342 (2.68), 8.346 (2.54), 13.534 (0.49).

The title compound (25 mg) was separated into enantiomers by preparative chiral HPLC to give enantiomer 1 (12 mg, see Example 43) and enantiomer 2 (10 mg, see Example 44).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; isocratic 40% B; Flow 50.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 43

### 3-Chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid-N-ethylethanamine Salt (Enantiomer 1)

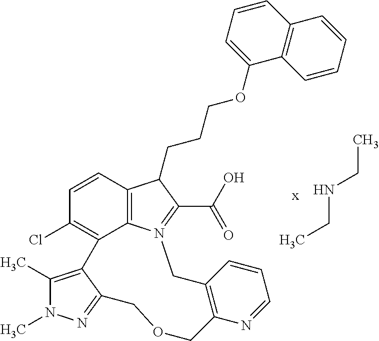

For the preparation of the racemic title compound see Example 42. Separation of enantiomers by preparative chiral HPLC (method see Example 42) gave the title compound (12 mg).

Analytical Chiral HPLC (method see Example 42): Rₜ=3.23 min.

LC-MS (Method 1): Rₜ=1.37 min; MS (ESIpos): m/z=607 [M+H]⁺

¹H-NMR (400 MHz, DMSO-d6) δ [ppm]: 1.133 (7.08), 1.152 (16.00), 1.170 (7.41), 1.230 (0.63), 1.829 (12.62), 1.905 (0.45), 2.107 (1.16), 2.125 (1.73), 2.142 (1.20), 2.332 (0.83), 2.518 (4.66), 2.522 (3.09), 2.673 (0.83), 2.858 (1.97), 2.876 (6.03), 2.894 (5.96), 2.912 (1.83), 3.119 (0.53), 3.133 (0.63), 3.152 (1.02), 3.173 (0.81), 3.192 (1.06), 3.209 (0.75), 3.225 (0.67), 3.242 (0.49), 3.826 (14.35), 4.042 (0.73), 4.049 (0.67), 4.066 (1.18), 4.081 (1.83), 4.105 (2.30), 4.121 (1.18), 4.138 (0.65), 4.145 (0.73), 4.212 (1.57), 4.244 (3.83), 4.267 (1.65), 4.412 (2.48), 4.444 (1.99), 5.019 (0.92), 5.055 (1.04), 5.673 (0.73), 5.708 (0.69), 6.787 (1.79), 6.805 (1.91), 7.082 (2.22), 7.103 (2.38), 7.119 (1.26), 7.131 (1.28), 7.138 (1.28), 7.150 (1.26), 7.345 (1.28), 7.365 (2.46), 7.384 (1.87), 7.427 (2.73), 7.448 (1.63), 7.463 (0.57), 7.467 (0.71), 7.480 (1.59), 7.484 (1.45), 7.495 (1.75), 7.501 (2.54), 7.504 (1.87), 7.515 (1.71), 7.519 (2.14), 7.525 (1.93), 7.532 (1.14), 7.536 (0.81), 7.546 (1.61), 7.782 (1.14), 7.801 (1.08), 7.841 (1.67), 7.846 (1.04), 7.859 (1.77), 7.863 (1.42), 8.191 (1.40), 8.195 (1.47), 8.213 (1.38), 8.260 (1.73), 8.264 (1.77), 8.272 (1.75), 8.276 (1.63).

### Example 44

### 3-Chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid-N-ethylethanamine Salt (Enantiomer 2)

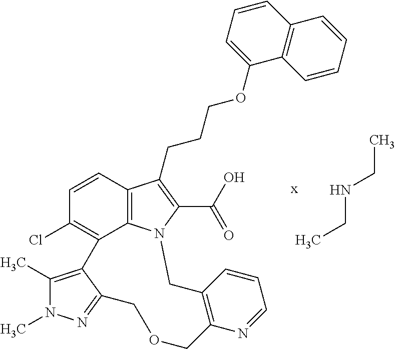

For the preparation of the racemic title compound see Example 42. Separation of enantiomers by preparative chiral HPLC (method see Example 42) gave the title compound (10 mg).

Analytical Chiral HPLC (method see Example 42): Rₜ=5.46 min.

LC-MS (Method 1): Rₜ=1.37 min, MS (ESIpos): m/z=607 [M+H]⁺

¹H-NMR (400 MHz, DMSO-d6) δ [ppm]: 1.138 (7.14), 1.156 (15.72), 1.174 (7.50), 1.230 (0.70), 1.834 (14.45), 2.106 (1.27), 2.123 (1.92), 2.140 (1.34), 2.157 (0.42), 2.332 (0.96), 2.336 (0.42), 2.518 (5.39), 2.522 (3.56), 2.673 (0.96), 2.678 (0.42), 2.866 (1.97), 2.884 (5.97), 2.902 (5.79), 2.921 (1.83), 3.119 (0.61), 3.133 (0.73), 3.152 (1.19), 3.174 (0.87), 3.193 (1.19), 3.211 (0.84), 3.226 (0.77), 3.245 (0.59), 3.826 (16.00), 4.044 (0.82), 4.051 (0.75), 4.067 (1.41), 4.077 (1.80), 4.102 (2.46), 4.119 (1.29), 4.136 (0.73), 4.143 (0.80), 4.207 (1.90), 4.240 (4.03), 4.266 (1.85), 4.413 (2.74), 4.445 (2.16), 5.035 (0.91), 5.072 (1.03), 5.648 (0.73), 5.684 (0.66), 6.789 (2.01), 6.807 (2.16), 7.093 (2.08), 7.114 (2.25), 7.127 (1.36), 7.139 (1.36), 7.147 (1.36), 7.158 (1.29), 7.346 (1.43), 7.366 (2.76), 7.385 (2.18), 7.428 (3.00), 7.449 (1.76), 7.463 (0.61), 7.468 (0.73), 7.480 (1.66), 7.484 (1.55), 7.495 (1.97), 7.501 (2.72), 7.504 (1.97), 7.515 (1.73), 7.519 (1.94), 7.532 (1.19), 7.539 (1.69), 7.560 (1.43), 7.767 (1.12), 7.786 (1.05), 7.841 (1.85), 7.846 (1.15), 7.860 (1.92), 7.864 (1.52), 8.190 (1.57), 8.195 (1.59), 8.212 (1.50), 8.265 (1.87), 8.269 (1.90), 8.277 (1.85), 8.281 (1.69).

### Example 45

### (rac)-3-Chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic Acid

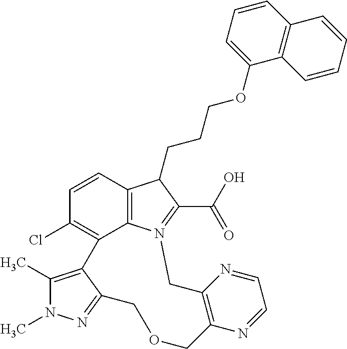

(rac)-Ethyl 3-chloro-4,5-dimethyl-17-{3-[(naphthalen-1-yl)oxy]propyl}-5,7,9,14-tetrahydropyrazino[2′,3′:3,4]pyrazolo[4′,3′:9,10][1,6]oxazacycloundecino[8,7,6-hi]indole-16-carboxylate (see intermediate 48, 275 mg, 50% purity, 216 μmol) was dissolved in a mixture of 3 mL of THF and 300 μL of ethanol and aqueous lithium hydroxide solution (430 μL, 1.0 M, 430 μmol) was added. The mixture was stirred at 70° C. for 48 hours in a sealed tube. Aqueous lithium hydroxide solution (430 μL, 1.0 M, 430 μmol) was added, and stirring was continued at 70° C. for 4 days in a sealed tube. Aqueous lithium hydroxide solution (215 μL, 1.0 M, 215 μmol) was added, and stirring was continued at 70° C. for 24 hours in a sealed tube. The reaction mixture was diluted with water and acidified using aqueous, saturated citric acid solution until a pH value of 3-4 was reached. The mixture was extracted with ethyl acetate and the combined organic layers were concentrated under reduced pressure. The crude material was purified by flash chromatography using silica gel (gradient dichloromethane/ethanol). The product was further purified by preparative HPLC (Method P1) to obtain the title compound (37.6 mg, 27% yield).

LC-MS (Method 1): Rₜ=1.43 min; MS (ESIpos): m/z=608 [M+H]⁺

¹H-NMR (400 MHz, DMSO-d6) δ [ppm]: 1.844 (16.00), 1.982 (0.61), 2.074 (0.69), 2.137 (1.04), 2.153 (1.55), 2.170 (1.11), 2.186 (0.40), 2.235 (0.61), 2.518 (2.90), 2.523 (1.82), 2.539 (0.69), 2.673 (0.54), 3.231 (1.03), 3.251 (1.63), 3.265 (0.98), 3.285 (0.44), 3.778 (0.55), 3.792 (0.64), 3.858 (14.60), 4.085 (0.73), 4.093 (0.67), 4.101 (0.52), 4.108 (1.14), 4.124 (0.48), 4.141 (0.51), 4.158 (1.15), 4.174 (0.68), 4.182 (0.86), 4.194 (1.88), 4.218 (1.87), 4.375 (1.82), 4.408 (2.18), 4.495 (1.74), 4.519 (1.53), 4.576 (1.83), 4.608 (1.44), 5.064 (1.42), 5.102 (1.79), 5.513 (1.58), 5.552 (1.48), 6.823 (1.84), 6.841 (2.00), 7.172 (4.22), 7.194 (4.15), 7.361 (1.49), 7.381 (2.69), 7.400 (2.21), 7.443 (2.74), 7.464 (1.57), 7.483 (0.47), 7.487 (0.69), 7.500 (1.68), 7.504 (1.59), 7.509 (1.92), 7.517 (3.84), 7.524 (1.96), 7.529 (1.74), 7.533 (1.93), 7.546 (0.78), 7.550 (0.46), 7.617 (3.80), 7.638 (3.27), 7.851 (1.61), 7.859 (0.93), 7.869 (1.55), 7.875 (1.45), 8.149 (0.42), 8.211 (1.42), 8.217 (1.31), 8.228 (0.73), 8.235 (1.33), 8.319 (3.16), 8.325 (3.94), 8.366 (4.72), 8.372 (3.77).

The title compound (30 mg) was separated into enantiomers by preparative chiral HPLC to give enantiomer 1 (9 mg, see Example 46) and enantiomer 2 (11 mg, see Example 47).

Preparative Chiral HPLC Method:

Instrument: Sepiatec: Prep SFC100; column: Chiralpak IG 5 μm 250×30 mm; Eluent A: CO₂, Eluent B: ethanol+0.2 Vol-% aqueous ammonia (32%); isocratic 33% B; Flow 100.0 mL/min Temperature: 40° C.; BPR: 150 bar; MWD @ 220 nm

Analytical Chiral HPLC Method:

Instrument: Agilent: 1260, Aurora SFC-Modul; column: Chiralpak IG 5 μm 100×4.6 mm; Eluent A: CO₂, Eluent B: ethanol+0.2 Vol-% aqueous ammonia (32%); isocratic 33% B; Flow 4.0 mL/min; Temperature: 37.5° C.; BPR: 100 bar; MWD @ 220 nm

### Example 46

### 3-Chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic Acid (Enantiomer 1)

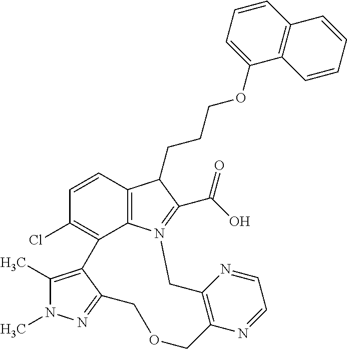

For the preparation of the racemic title compound see Example 45. Separation of enantiomers by preparative chiral HPLC (method see Example 45) gave the title compound (9 mg).

Analytical Chiral HPLC (method see Example 45): Rₜ=1.84 min.

LC-MS (Method 1): Rₜ=1.43 min, MS (ESIpos): m/z=608 [M+H]⁺

¹H-NMR (400 MHz, DMSO-d6) δ [ppm]: 0.851 (0.42), 0.859 (0.82), 0.967 (0.58), 1.108 (16.00), 1.117 (0.59), 1.144 (0.59), 1.849 (8.11), 2.138 (0.62), 2.156 (0.92), 2.171 (0.65), 2.327 (0.65), 2.331 (0.48), 2.518 (2.82), 2.523 (1.79), 2.669 (0.68), 2.673 (0.50), 3.230 (0.63), 3.249 (0.99), 3.265 (0.65), 3.844 (6.27), 4.111 (0.59), 4.159 (0.62), 4.175 (0.41), 4.183 (0.47), 4.192 (0.72), 4.214 (0.63), 4.239 (0.71), 4.361 (0.82), 4.393 (1.03), 4.483 (0.90), 4.508 (0.78), 4.549 (0.65), 4.581 (0.50), 5.048 (0.62), 5.087 (0.75), 5.533 (0.59), 5.571 (0.54), 6.823 (1.01), 6.842 (1.08), 7.164 (1.90), 7.185 (1.99), 7.360 (0.75), 7.381 (1.45), 7.400 (1.14), 7.442 (1.64), 7.463 (0.94), 7.499 (0.87), 7.504 (0.87), 7.509 (1.06), 7.516 (1.99), 7.523 (1.01), 7.528 (0.99), 7.533 (1.03), 7.609 (1.56), 7.631 (1.36), 7.851 (0.97), 7.859 (0.54), 7.869 (0.89), 7.875 (0.83), 8.211 (0.77), 8.217 (0.73), 8.235 (0.73), 8.318 (1.68), 8.324 (2.00), 8.362 (1.78), 8.368 (1.42).

### Example 47

### 3-Chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic Acid (Enantiomer 2)

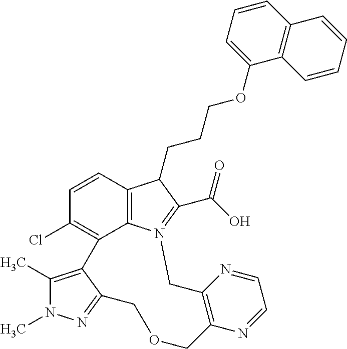

For the preparation of the racemic title compound see Example 45. Separation of enantiomers by preparative chiral HPLC (method see Example 45) gave the title compound (11 mg).

Analytical Chiral HPLC (method see Example 45): Rₜ=6.82 min.

LC-MS (Method 1): Rₜ=1.43 min, MS (ESIpos): m/z=608 [M+H]⁺

¹H-NMR (400 MHz, DMSO-d6) δ [ppm]: 0.704 (0.90), 0.722 (2.26), 0.740 (1.06), 0.765 (0.50), 0.782 (0.43), 0.794 (0.61), 0.806 (0.88), 0.831 (2.07), 0.842 (3.30), 0.850 (8.19), 0.858 (6.43), 0.869 (2.60), 0.875 (4.20), 0.890 (0.64), 0.896 (0.53), 0.902 (0.90), 0.918 (0.48), 0.960 (0.74), 0.967 (1.57), 0.977 (0.72), 1.029 (3.67), 1.039 (0.72), 1.060 (0.56), 1.070 (0.66), 1.080 (0.48), 1.085 (0.61), 1.108 (9.59), 1.125 (3.85), 1.138 (3.64), 1.140 (4.01), 1.156 (0.58), 1.172 (0.48), 1.185 (0.58), 1.208 (0.61), 1.231 (0.69), 1.409 (0.58), 1.427 (1.49), 1.446 (1.44), 1.465 (0.50), 1.856 (16.00), 1.976 (0.48), 1.996 (1.17), 2.009 (0.40), 2.015 (0.50), 2.026 (1.30), 2.093 (0.74), 2.124 (1.28), 2.141 (1.46), 2.158 (1.86), 2.175 (1.38), 2.195 (0.66), 2.252 (0.61), 2.284 (0.56), 2.301 (0.45), 2.331 (1.44), 2.518 (6.22), 2.523 (4.04), 2.539 (0.56), 2.651 (0.40), 2.673 (1.25), 3.233 (1.38), 3.249 (2.13), 3.266 (1.57), 3.828 (10.84), 4.089 (0.77), 4.097 (0.74), 4.113 (1.22), 4.129 (0.56), 4.146 (0.58), 4.162 (1.25), 4.177 (0.77), 4.185 (0.88), 4.239 (1.12), 4.264 (1.33), 4.344 (1.44), 4.375 (1.86), 4.471 (1.73), 4.496 (1.54), 4.519 (1.20), 4.550 (0.85), 5.031 (1.20), 5.069 (1.44), 5.558 (1.14), 5.596 (1.01), 6.825 (2.05), 6.843 (2.18), 7.154 (3.32), 7.176 (3.48), 7.359 (1.51), 7.379 (2.84), 7.398 (2.21), 7.441 (3.14), 7.462 (1.78), 7.481 (0.48), 7.486 (0.69), 7.498 (1.78), 7.503 (1.70), 7.508 (2.07), 7.516 (3.88), 7.523 (2.05), 7.528 (1.83), 7.532 (1.94), 7.545 (0.74), 7.549 (0.45), 7.600 (3.14), 7.622 (2.71), 7.851 (1.86), 7.858 (1.01), 7.868 (1.73), 7.874 (1.54), 8.211 (1.54), 8.217 (1.49), 8.234 (1.44), 8.317 (3.35), 8.323 (4.07), 8.356 (3.85), 8.362 (2.98).

### Example 48

### (rac)-3-Chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic Acid

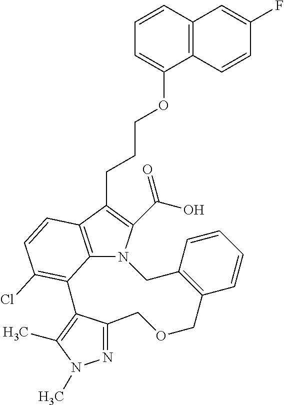 [SMILES: Cc1c2c(nn1C)COCc1ccccc1Cn1c(C(=O)O)c(CCCOc3cccc4cc(F)ccc34)c3ccc(Cl)c-2c31]

(rac)-Ethyl 3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylate (see intermediate 50, 177 mg, 271 μmol) was dissolved in a mixture of 10 mL of THF and 5 mL of ethanol, and aqueous lithium hydroxide solution (540 μL, 1.0 M, 540 μmol) was added. The mixture was stirred at 70° C. for 3 days. Aqueous lithium hydroxide solution (200 μL, 1.0 M, 200 μmol) was added, and stirring was continued at 70° C. for 72 hours. After concentration, water and aqueous saturated citric acid solution were added until a pH value of 5 was reached. The mixture was extracted with ethyl acetate and the combined organic layers were dried over sodium sulfate, filtered and concentrated under reduced pressure to give the title compound (172 mg, 99% yield).

LC-MS (Method 1): Rₜ=1.55 min, MS (ESIpos): m/z=624 [M+H]⁺

¹H-NMR (400 MHz, DMSO-d6) δ [ppm]: 0.000 (0.56), 0.884 (0.97), 1.199 (2.04), 1.217 (4.41), 1.234 (2.12), 1.277 (1.22), 1.907 (15.36), 1.952 (2.14), 2.032 (6.84), 2.136 (1.53), 2.152 (2.30), 2.168 (1.63), 2.372 (1.48), 2.715 (1.51), 3.171 (0.69), 3.186 (0.84), 3.205 (1.30), 3.224 (0.64), 3.240 (0.66), 3.260 (1.28), 3.277 (0.92), 3.293 (0.82), 3.312 (0.48), 3.883 (16.00), 3.987 (1.68), 4.013 (2.42), 4.046 (0.61), 4.063 (1.71), 4.081 (1.79), 4.099 (1.20), 4.121 (3.39), 4.147 (3.14), 4.163 (3.14), 4.194 (2.63), 4.455 (2.70), 4.487 (2.30), 5.256 (1.12), 5.294 (2.58), 5.341 (2.25), 5.377 (1.10), 6.814 (1.51), 6.820 (1.74), 6.830 (1.61), 6.835 (1.84), 7.145 (1.48), 7.161 (2.58), 7.184 (0.74), 7.195 (1.51), 7.206 (2.07), 7.230 (6.61), 7.239 (3.16), 7.277 (3.14), 7.298 (3.34), 7.382 (0.89), 7.388 (1.05), 7.404 (1.63), 7.411 (1.96), 7.427 (0.97), 7.433 (1.12), 7.444 (0.79), 7.465 (2.63), 7.481 (6.46), 7.496 (0.71), 7.681 (1.71), 7.687 (1.99), 7.707 (1.71), 7.713 (1.91), 7.757 (3.04), 7.777 (2.70), 8.253 (1.48), 8.268 (1.61), 8.276 (1.66), 8.291 (1.48).

The title compound (164 mg) was separated into enantiomers by preparative chiral HPLC to give enantiomer 1 (41 mg, see Example 49) and enantiomer 2 (56 mg, see Example 50).

Preparative Chiral HPLC Method:

Instrument: Labomatic HD5000, Labocord-5000; Gilson GX-241, Labcol Vario 4000, column: Chiralpak ID 5μ 250×30 mm; Eluent A: hexane+0.1 Vol-% N-ethylethanamine (99%); Eluent B: ethanol; Isocratic: 75% A+25% B; flow 50.0 mL/min; UV 254 nm

Analytical Chiral HPLC Method:

Instrument: Agilent HPLC 1260; column: Chiralpak ID 3μ 100×4.6 mm; Eluent A: hexane+0.1 Vol-% N-ethylethanamine (99%); Eluent B: ethanol; Gradient: 20-50% B in 7 min; flow 1.4 mL/min; Temperature: 25° C.; DAD 254 nm

### Example 49

### (−)-3-Chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine Salt (Enantiomer 1)

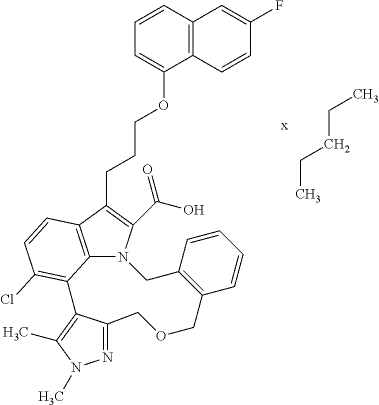

For the preparation of the racemic title compound see Example 48. Separation of enantiomers by preparative chiral HPLC (method see Example 48) gave the title compound (41 mg).

Analytical Chiral HPLC (method see Example 48): Rₜ=2.84 min.

LC-MS (Method 1): Rₜ=1.53 min; MS (ESIpos): m/z=624 [M+H]⁺

Specific Optical Rotation (Method O1): −38.7° (c=10 mg/mL, CHCl₃)

¹H-NMR (400 MHz, DMSO-d6) δ [ppm]: 0.798 (1.25), 0.803 (0.64), 0.815 (1.37), 0.822 (1.36), 0.840 (0.81), 0.852 (0.59), 0.886 (0.64), 0.904 (1.32), 0.922 (0.70), 1.035 (0.66), 1.053 (1.14), 1.070 (0.68), 1.128 (7.33), 1.145 (16.00), 1.164 (7.59), 1.236 (1.19), 1.259 (0.42), 1.792 (10.83), 2.120 (0.90), 2.137 (1.34), 2.154 (0.95), 2.170 (0.44), 2.327 (1.08), 2.332 (0.79), 2.412 (0.48), 2.518 (7.59), 2.522 (4.67), 2.669 (1.10), 2.673 (0.81), 2.831 (2.00), 2.849 (6.14), 2.867 (5.96), 2.885 (1.83), 3.129 (0.42), 3.143 (0.60), 3.162 (1.26), 3.185 (1.10), 3.203 (0.70), 3.218 (0.53), 3.237 (0.44), 3.834 (12.26), 4.037 (1.10), 4.062 (2.05), 4.080 (0.95), 4.097 (0.44), 4.128 (1.80), 4.138 (1.19), 4.148 (2.07), 4.153 (1.78), 4.180 (1.91), 4.381 (1.92), 4.413 (1.56), 4.980 (0.77), 5.016 (0.90), 5.669 (0.57), 5.705 (0.53), 6.771 (1.01), 6.778 (1.08), 6.786 (0.95), 6.793 (1.08), 7.052 (2.16), 7.060 (1.36), 7.074 (4.22), 7.078 (5.64), 7.087 (1.78), 7.341 (0.71), 7.348 (0.84), 7.364 (1.85), 7.370 (2.02), 7.386 (1.45), 7.393 (1.03), 7.409 (1.91), 7.418 (2.22), 7.425 (4.53), 7.439 (0.42), 7.495 (1.41), 7.516 (1.28), 7.627 (1.23), 7.634 (1.26), 7.653 (1.25), 7.660 (1.23), 8.231 (1.06), 8.246 (1.14), 8.254 (1.12), 8.269 (1.01)

### Example 50

### (+)-3-Chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine Salt (Enantiomer 2)

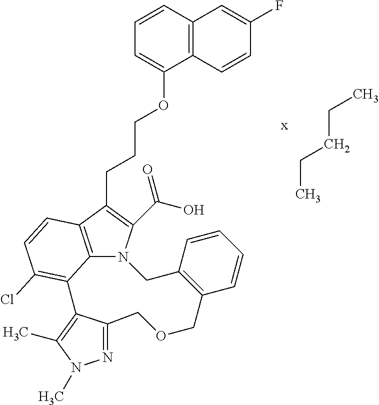

For the preparation of the racemic title compound see Example 48. Separation of enantiomers by preparative chiral HPLC (method see Example 48) gave the title compound (56 mg).

Analytical Chiral HPLC (method see Example 48): Rₜ=4.07 min.

LC-MS (Method 1): Rₜ=1.52 min, MS (ESIpos): m/z=624 [M+H]⁺

Specific Optical Rotation (Method O1): +46.5° (c=10 mg/mL, CHCl₃)

¹H-NMR (400 MHz, DMSO-d6) δ [ppm]: 0.798 (0.99), 0.803 (0.56), 0.815 (1.18), 0.822 (1.18), 0.835 (0.62), 0.840 (0.78), 0.852 (0.88), 0.886 (0.52), 0.905 (0.99), 0.922 (0.50), 1.053 (0.52), 1.134 (7.43), 1.152 (16.00), 1.170 (7.58), 1.235 (1.83), 1.259 (0.65), 1.795 (12.04), 2.117 (1.10), 2.136 (1.57), 2.153 (1.14), 2.171 (0.49), 2.518 (5.86), 2.523 (3.77), 2.843 (2.05), 2.862 (6.33), 2.880 (6.11), 2.898 (1.94), 3.129 (0.49), 3.143 (0.71), 3.166 (5.40), 3.188 (1.21), 3.205 (0.78), 3.221 (0.60), 3.238 (0.43), 3.834 (13.39), 4.033 (1.19), 4.058 (2.39), 4.080 (1.16), 4.097 (0.62), 4.124 (2.18), 4.136 (1.40), 4.147 (2.69), 4.179 (2.18), 4.383 (2.17), 4.415 (1.75), 4.993 (0.86), 5.029 (0.97), 5.650 (0.60), 5.687 (0.56), 6.770 (1.18), 6.777 (1.19), 6.786 (1.12), 6.792 (1.23), 7.062 (2.39), 7.082 (8.42), 7.090 (2.00), 7.341 (0.84), 7.347 (1.10), 7.363 (2.24), 7.369 (1.96), 7.385 (1.12), 7.392 (1.03), 7.409 (2.09), 7.419 (2.56), 7.425 (4.93), 7.439 (0.45), 7.506 (1.46), 7.527 (1.31), 7.627 (1.36), 7.634 (1.38), 7.653 (1.40), 7.660 (1.34), 8.229 (1.18), 8.245 (1.27), 8.253 (1.21), 8.268 (1.12).

### Example 51

### (rac)-3-Chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic Acid

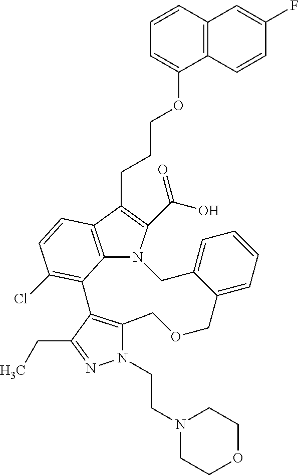 [SMILES: CCc1nn(CCN2CCOCC2)c2c1-c1c(Cl)ccc3c(CCCOc4cccc5cc(F)ccc45)c(C(=O)O)n(c13)Cc1ccccc1COC2]

To a solution of (rac)-ethyl 3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylate (see intermediate 54, 330 mg, 431 μmol) in a mixture of THF (8.8 mL) and ethanol (4.4 mL) was added an aqueous solution of lithium hydroxide (4.4 mL, 1.0 M, 4.4 mmol). The resulting mixture was stirred at rt for 10 days. After removal of all volatiles, the residue was subjected to flash chromatography (silica gel, dichloromethane/ethanol gradient) to give the title compound (302 mg, 87% yield).

LC-MS (Method 2): Rₜ=0.98 min, MS (ESIpos): m/z=737 [M+H]⁺

¹H-NMR (400 MHz, DMSO-d6) δ [ppm]: 0.841 (3.88), 0.860 (8.50), 0.879 (4.11), 0.902 (0.59), 1.035 (7.30), 1.053 (16.00), 1.071 (7.84), 1.232 (0.55), 2.065 (1.73), 2.084 (3.86), 2.102 (2.89), 2.121 (1.14), 2.132 (1.19), 2.144 (2.18), 2.169 (2.84), 2.181 (2.08), 2.323 (0.76), 2.327 (1.04), 2.332 (0.79), 2.375 (1.24), 2.387 (1.12), 2.402 (1.11), 2.438 (0.42), 2.523 (3.66), 2.551 (0.62), 2.566 (0.79), 2.582 (0.82), 2.597 (1.02), 2.611 (0.52), 2.669 (1.46), 2.673 (1.02), 2.687 (1.06), 2.705 (0.67), 2.718 (0.69), 3.175 (0.47), 3.191 (0.67), 3.209 (1.04), 3.230 (0.96), 3.249 (1.14), 3.268 (0.79), 3.283 (0.77), 3.404 (3.76), 3.415 (6.58), 3.425 (4.99), 3.441 (2.18), 3.451 (1.85), 3.469 (0.64), 3.874 (1.70), 3.909 (1.71), 3.986 (1.01), 4.014 (1.65), 4.083 (2.10), 4.095 (0.97), 4.112 (2.95), 4.128 (1.83), 4.143 (0.79), 4.152 (0.54), 4.275 (1.11), 4.289 (2.20), 4.305 (1.33), 4.356 (1.36), 4.758 (1.68), 4.793 (1.61), 5.195 (1.02), 5.234 (1.61), 5.357 (1.01), 5.396 (0.72), 6.784 (1.36), 6.789 (1.41), 6.801 (1.38), 6.805 (1.44), 6.918 (1.34), 6.937 (1.61), 7.096 (0.52), 7.103 (0.59), 7.110 (0.84), 7.117 (1.07), 7.129 (0.77), 7.137 (0.87), 7.147 (0.62), 7.169 (2.50), 7.173 (2.95), 7.181 (3.44), 7.199 (2.65), 7.221 (2.67), 7.331 (0.77), 7.338 (0.89), 7.354 (1.36), 7.361 (1.48), 7.376 (0.84), 7.383 (0.91), 7.396 (0.60), 7.417 (2.10), 7.433 (4.53), 7.452 (0.62), 7.638 (1.48), 7.644 (1.53), 7.664 (1.49), 7.670 (1.49), 7.734 (2.33), 7.755 (2.10), 8.224 (1.28), 8.239 (1.36), 8.247 (1.34), 8.262 (1.24).

The title compound (290 mg) was separated into enantiomers by preparative chiral HPLC to give enantiomer 1 (117 mg, see Example 52) and enantiomer 2 (123 mg, see Example 53).

Preparative Chiral HPLC Method:

Instrument: Labomatic HD5000, Labocord-5000; Gilson GX-241, Labcol Vario 4000, column: Chiralpak ID 5μ 250×30 mm; Eluent A: hexane+0.1 Vol-% trifluoroacetic acid; Eluent B: 2-propanol; isocratic 70% A+30% B; Flow 50.0 mL/min; UV 254 nm

Analytical Chiral HPLC Method:

Instrument: Agilent HPLC 1260; column: Chiralpak ID 3μ 100×4.6 mm; Eluent A: hexane+0.1 Vol-% trifluoroacetic acid; Eluent B: 2-propanol; Gradient: 20-50% B in 7 min; Flow 1.4 mL/min; Temperature: 25° C.; DAD 254 nm

### Example 52

### (−)-3-Chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic Acid (Enantiomer 1)

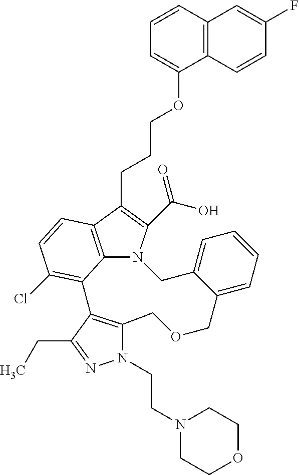 [SMILES: CCc1nn(CCN2CCOCC2)c2c1-c1c(Cl)ccc3c(CCCOc4cccc5cc(F)ccc45)c(C(=O)O)n(c13)Cc1ccccc1COC2]

For the preparation of the racemic title compound see Example 51. Separation of enantiomers by preparative chiral HPLC (method see Example 51) gave the title compound (117 mg).

Analytical Chiral HPLC (method see Example 51): Rₜ=3.67 min.

Enantiomer 1 was further purified by addition of water, subsequent lyophilisation and flash chromatography using silica gel (gradient dichloromethane/ethanol) to give the title compound (79 mg).

LC-MS (Method 2): Rₜ=1.01 min, MS (ESIpos): m/z=737 [M+H]⁺

Specific Optical Rotation (Method O1): −32.7° (c=10 mg/mL, DMSO)

¹H-NMR (400 MHz, DMSO-d6) δ [ppm]: 0.798 (1.27), 0.803 (0.55), 0.815 (1.45), 0.822 (1.45), 0.841 (7.64), 0.860 (16.00), 0.879 (7.33), 0.886 (1.15), 0.905 (1.64), 0.922 (0.79), 1.232 (1.45), 2.062 (1.64), 2.081 (4.85), 2.100 (4.73), 2.119 (1.70), 2.132 (1.76), 2.144 (3.39), 2.170 (4.42), 2.182 (3.33), 2.336 (1.15), 2.375 (2.00), 2.389 (1.82), 2.406 (1.58), 2.413 (1.27), 2.518 (14.00), 2.523 (9.45), 2.552 (0.67), 2.566 (1.09), 2.582 (1.21), 2.597 (1.58), 2.611 (0.73), 2.678 (1.45), 2.687 (1.76), 2.705 (1.09), 2.718 (1.15), 2.737 (0.48), 3.175 (0.73), 3.191 (1.03), 3.209 (1.70), 3.228 (1.52), 3.248 (1.82), 3.266 (1.15), 3.282 (1.09), 3.403 (5.39), 3.415 (9.76), 3.426 (5.39), 3.874 (3.03), 3.909 (3.03), 3.987 (1.45), 4.015 (2.42), 4.084 (3.52), 4.096 (1.58), 4.112 (4.91), 4.129 (2.97), 4.144 (1.27), 4.153 (0.91), 4.275 (1.70), 4.289 (3.58), 4.305 (2.12), 4.758 (2.91), 4.792 (2.79), 5.192 (1.39), 5.230 (2.18), 5.361 (1.09), 5.399 (0.79), 5.759 (5.33), 6.785 (2.30), 6.789 (2.42), 6.801 (2.30), 6.806 (2.48), 6.918 (2.18), 6.937 (2.61), 7.094 (0.79), 7.101 (0.85), 7.109 (1.33), 7.116 (1.64), 7.127 (1.15), 7.135 (1.27), 7.146 (0.97), 7.167 (3.76), 7.173 (4.79), 7.180 (5.76), 7.197 (3.45), 7.218 (3.39), 7.332 (1.52), 7.338 (1.70), 7.354 (2.36), 7.361 (2.61), 7.376 (1.58), 7.383 (1.76), 7.397 (1.03), 7.418 (3.64), 7.434 (7.58), 7.452 (1.03), 7.638 (2.73), 7.644 (2.85), 7.664 (2.73), 7.670 (2.79), 7.731 (2.79), 7.752 (2.61), 8.224 (2.30), 8.239 (2.42), 8.247 (2.36), 8.262 (2.24).

### Example 53

### (+)-3-Chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic Acid (Enantiomer 2)

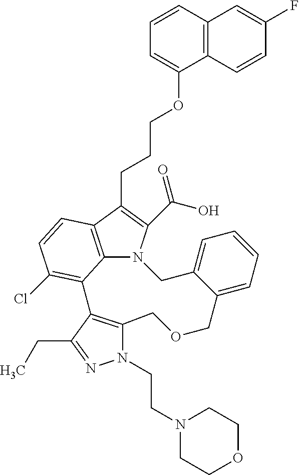 [SMILES: CCc1nn(CCN2CCOCC2)c2c1-c1c(Cl)ccc3c(CCCOc4cccc5cc(F)ccc45)c(C(=O)O)n(c13)Cc1ccccc1COC2]

For the preparation of the racemic title compound see Example 51. Separation of enantiomers by preparative chiral HPLC (method see Example 51) gave the title compound (123 mg).

Analytical Chiral HPLC (method see Example 51): Rₜ=6.42 min.

Enantiomer 2 was further purified by addition of water, subsequent lyophilisation and flash chromatography using silica gel (gradient dichloromethane/ethanol) to give the title compound (60 mg).

LC-MS (Method 2): Rₜ=0.99 min, MS (ESIpos): m/z=737 [M+H]⁺

Specific Optical Rotation (Method O1): +34.2° (c=10 mg/mL, DMSO)

¹H-NMR (400 MHz, DMSO-d6) δ [ppm]: 0.815 (0.54), 0.822 (0.60), 0.840 (7.46), 0.859 (15.94), 0.877 (7.70), 0.904 (0.54), 1.232 (2.05), 2.061 (1.80), 2.080 (5.35), 2.099 (5.23), 2.118 (1.98), 2.145 (4.27), 2.171 (5.35), 2.183 (4.15), 2.322 (2.59), 2.327 (3.55), 2.332 (2.77), 2.377 (2.41), 2.389 (2.23), 2.404 (2.11), 2.522 (16.00), 2.567 (1.74), 2.582 (1.68), 2.598 (2.05), 2.612 (1.08), 2.665 (2.89), 2.669 (4.39), 2.673 (3.31), 2.687 (2.17), 2.705 (1.32), 2.719 (1.32), 2.738 (0.60), 3.176 (0.90), 3.191 (1.26), 3.210 (1.98), 3.229 (1.86), 3.248 (2.17), 3.265 (1.50), 3.282 (1.38), 3.404 (6.56), 3.415 (11.61), 3.426 (6.74), 3.874 (3.31), 3.909 (3.37), 3.989 (1.62), 4.017 (2.77), 4.084 (4.15), 4.096 (1.98), 4.112 (5.71), 4.129 (3.61), 4.145 (1.56), 4.275 (2.17), 4.289 (4.33), 4.305 (2.65), 4.757 (3.25), 4.792 (3.13), 5.190 (1.68), 5.228 (2.47), 5.366 (1.14), 5.404 (0.90), 5.759 (6.26), 6.784 (2.59), 6.789 (2.83), 6.801 (2.71), 6.806 (2.95), 6.918 (2.59), 6.937 (3.19), 7.114 (1.92), 7.125 (1.38), 7.133 (1.44), 7.145 (1.14), 7.167 (4.27), 7.173 (5.59), 7.180 (6.92), 7.194 (3.91), 7.216 (3.55), 7.332 (1.56), 7.338 (1.74), 7.354 (2.65), 7.361 (2.95), 7.376 (1.68), 7.383 (1.86), 7.396 (1.20), 7.417 (4.09), 7.434 (8.96), 7.452 (1.26), 7.638 (2.89), 7.644 (3.13), 7.664 (2.95), 7.670 (3.07), 7.729 (2.95), 7.749 (2.71), 8.224 (2.47), 8.240 (2.65), 8.248 (2.71), 8.262 (2.47).

### Example 54

### (rac)-3-Chloro-4-ethyl-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic Acid

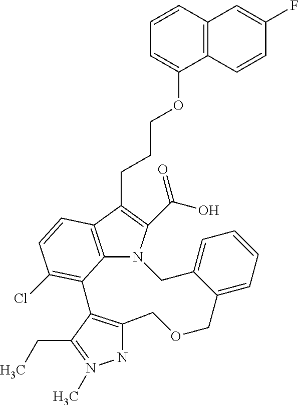

To a solution of (rac)-ethyl 3-chloro-4-ethyl-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylate (see intermediate 56, 375 mg, 563 μmol) in a mixture of THF (9.3 mL) and ethanol (4.7 mL) was added an aqueous solution of lithium hydroxide (4.7 mL, 1.0 M, 4.7 mmol). The resulting mixture was stirred at 40° C. for 40 hours, followed by stirring at 70° C. for 6 hours. The mixture was neutralized using 2-molar aqueous hydrochloric acid. After removal of all volatiles, the residue was subjected to flash chromatography (silica gel, dichloromethane/ethanol gradient) to give the title compound (369 mg, 92% yield).

LC-MS (Method 2): Rₜ=0.97 min, MS (ESIpos): m/z=638 [M+H]⁺

¹H-NMR (400 MHz, DMSO-d6) δ [ppm]: 0.860 (2.94), 0.879 (6.79), 0.898 (3.19), 0.901 (2.37), 0.920 (0.74), 1.035 (6.91), 1.052 (12.92), 1.070 (6.87), 1.172 (0.62), 1.232 (0.66), 1.757 (0.58), 2.065 (1.77), 2.086 (1.03), 2.103 (1.56), 2.119 (1.07), 2.137 (0.39), 2.166 (0.49), 2.185 (0.76), 2.203 (1.07), 2.222 (0.88), 2.242 (0.47), 2.260 (0.82), 2.279 (0.97), 2.297 (0.74), 2.518 (6.83), 2.522 (4.03), 3.126 (0.47), 3.140 (0.56), 3.160 (0.99), 3.179 (0.82), 3.198 (0.95), 3.217 (0.58), 3.231 (0.49), 3.405 (0.51), 3.417 (0.62), 3.422 (1.48), 3.434 (1.54), 3.440 (1.54), 3.452 (1.48), 3.457 (0.62), 3.469 (0.47), 3.599 (0.45), 3.868 (16.00), 3.946 (1.21), 3.971 (1.71), 4.035 (0.60), 4.044 (0.68), 4.059 (1.23), 4.080 (2.28), 4.087 (1.54), 4.105 (1.99), 4.113 (2.28), 4.145 (1.87), 4.344 (0.72), 4.357 (1.34), 4.370 (0.70), 4.414 (2.20), 4.446 (1.89), 5.220 (0.51), 5.257 (1.65), 5.282 (1.30), 5.319 (0.47), 5.759 (1.79), 6.747 (1.25), 6.752 (1.38), 6.763 (1.21), 6.768 (1.36), 7.092 (0.88), 7.097 (1.15), 7.113 (1.75), 7.139 (0.56), 7.144 (0.58), 7.155 (1.09), 7.161 (1.44), 7.169 (1.03), 7.174 (1.01), 7.179 (1.23), 7.188 (1.62), 7.192 (1.73), 7.205 (2.53), 7.228 (2.82), 7.250 (2.67), 7.335 (0.80), 7.342 (0.90), 7.358 (1.28), 7.364 (1.42), 7.380 (0.86), 7.386 (0.97), 7.393 (0.62), 7.414 (1.97), 7.431 (4.20), 7.448 (0.58), 7.634 (1.42), 7.641 (1.56), 7.660 (1.46), 7.667 (1.54), 7.707 (2.16), 7.729 (1.93), 8.210 (1.28), 8.225 (1.32), 8.233 (1.28), 8.248 (1.21).

The title compound (360 mg) was separated into enantiomers by preparative chiral HPLC to give enantiomer 1 (162 mg, see Example 55) and enantiomer 2 (133 mg, see Example 56).

Preparative Chiral HPLC Method:

Instrument: Sepiatec: Prep SFC100; column: Chiralpak IG 5 μm 250×30 mm; Eluent A: CO₂, Eluent B: 2-propanol+0.4 Vol-% N-ethylethanamine (99%); isocratic 34% B; Flow 100.0 mL/min Temperature: 40° C.; BPR: 150 bar; MWD 220 nm

Analytical Chiral HPLC Method:

Instrument: Agilent: 1260, Aurora SFC-Modul; column: Chiralpak IG 5 μm 100×4.6 mm; Eluent A: CO₂, Eluent B: 2-propanol+0.2 Vol-% N-ethylethanamine (99%); isocratic 34% B; Flow 4.0 mL/min; Temperature: 37.5° C.; BPR: 100 bar; MWD 220 nm

### Example 55

### (−)-3-Chloro-4-ethyl-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic Acid N-ethylethanamine Salt (Enantiomer 1)

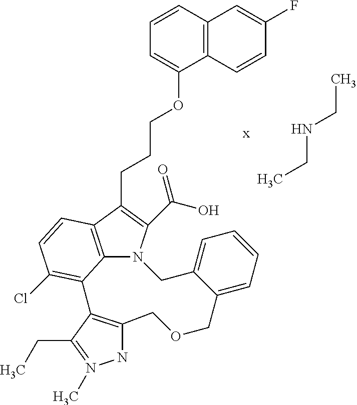

For the preparation of the racemic title compound see Example 54. Separation of enantiomers by preparative chiral HPLC (method see Example 54) gave the title compound (162 mg).

Analytical Chiral HPLC (method see Example 54): Rₜ=1.67 min.

Enantiomer 1 was further purified by addition of water and subsequent lyophilisation to give the title compound (140 mg).

LC-MS (Method 1): Rₜ=1.58 min, MS (ESIpos): m/z=638 [M+H]⁺

Specific Optical Rotation (Method O1): −86.1° (c=10 mg/mL, DMSO)

¹H-NMR (400 MHz, DMSO-d6) δ [ppm]: 0.821 (2.86), 0.840 (6.58), 0.859 (3.00), 1.026 (0.57), 1.042 (0.56), 1.142 (7.37), 1.160 (16.00), 1.178 (7.61), 2.099 (0.40), 2.118 (1.32), 2.135 (2.00), 2.154 (2.08), 2.174 (1.42), 2.197 (1.06), 2.214 (0.57), 2.518 (3.00), 2.523 (1.89), 2.855 (1.96), 2.872 (6.14), 2.891 (6.02), 2.909 (1.81), 3.157 (0.93), 3.169 (1.60), 3.175 (1.57), 3.190 (1.00), 3.207 (0.40), 3.860 (15.01), 4.031 (1.29), 4.037 (0.93), 4.045 (0.77), 4.056 (2.20), 4.078 (0.47), 4.102 (0.48), 4.119 (1.26), 4.127 (2.21), 4.143 (0.95), 4.151 (2.86), 4.183 (1.96), 4.388 (2.22), 4.420 (1.81), 5.011 (0.81), 5.048 (0.93), 5.632 (0.55), 5.668 (0.51), 6.746 (1.25), 6.751 (1.29), 6.762 (1.22), 6.767 (1.32), 7.067 (2.16), 7.075 (2.65), 7.080 (4.99), 7.086 (8.72), 7.338 (0.81), 7.344 (0.92), 7.360 (1.32), 7.367 (1.54), 7.382 (1.81), 7.389 (1.76), 7.401 (2.58), 7.417 (3.98), 7.436 (0.55), 7.514 (1.52), 7.535 (1.36), 7.626 (1.45), 7.632 (1.49), 7.652 (1.46), 7.658 (1.45), 8.227 (1.24), 8.242 (1.30), 8.250 (1.28), 8.264 (1.20).

### Example 56

### (+)-3-Chloro-4-ethyl-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic Acid N-ethylethanamine Salt (Enantiomer 2)

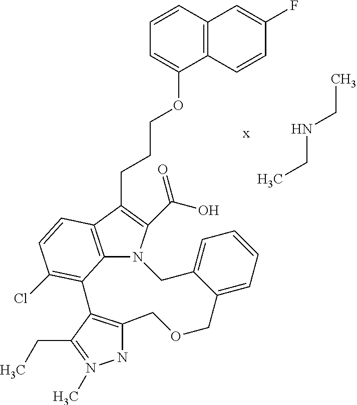

For the preparation of the racemic title compound see Example 54. Separation of enantiomers by preparative chiral HPLC (method see Example 54) gave the title compound (133 mg).

Analytical Chiral HPLC (method see Example 54): Rₜ=3.21 min.

Enantiomer 2 was further purified by addition of water and subsequent lyophilisation to give the title compound (119 mg).

LC-MS (Method 1): Rₜ=1.58 min, MS (ESIpos): m/z=638 [M+H]⁺

Specific Optical Rotation (Method O1): +92.3° (c=10 mg/mL, DMSO)

¹H-NMR (400 MHz, DMSO-d6) δ [ppm]: 0.822 (2.78), 0.841 (6.44), 0.859 (2.89), 1.141 (7.13), 1.160 (16.00), 1.177 (7.18), 2.116 (1.16), 2.134 (1.69), 2.156 (1.66), 2.175 (1.28), 2.199 (1.02), 2.216 (0.55), 2.331 (0.66), 2.518 (3.19), 2.523 (2.08), 2.673 (0.64), 2.857 (1.89), 2.875 (6.01), 2.893 (5.83), 2.911 (1.74), 3.157 (0.88), 3.169 (1.53), 3.175 (1.50), 3.189 (0.92), 3.861 (15.72), 4.028 (1.19), 4.038 (0.86), 4.053 (2.08), 4.061 (1.22), 4.078 (0.47), 4.101 (0.48), 4.118 (1.27), 4.125 (2.19), 4.143 (0.95), 4.150 (2.78), 4.182 (1.92), 4.389 (2.19), 4.420 (1.78), 5.017 (0.69), 5.054 (0.80), 5.622 (0.44), 5.658 (0.41), 6.746 (1.24), 6.751 (1.28), 6.763 (1.19), 6.768 (1.28), 7.072 (1.99), 7.082 (5.08), 7.089 (7.13), 7.338 (0.81), 7.344 (0.94), 7.360 (1.41), 7.367 (1.80), 7.382 (1.91), 7.389 (1.63), 7.402 (2.16), 7.419 (3.99), 7.436 (0.56), 7.520 (1.20), 7.541 (1.09), 7.626 (1.42), 7.633 (1.45), 7.652 (1.44), 7.659 (1.42), 8.227 (1.24), 8.241 (1.28), 8.249 (1.25), 8.264 (1.19).

### Example 57

### (rac)-3-Chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic Acid

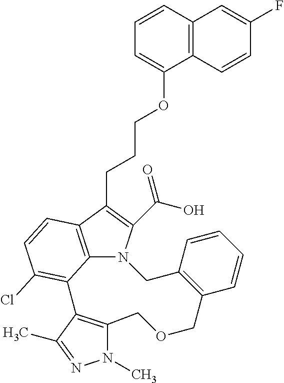 [SMILES: Cc1nn(C)c2c1-c1c(Cl)ccc3c(CCCOc4cccc5cc(F)ccc45)c(C(=O)O)n(c13)Cc1ccccc1COC2]

To a solution of (rac)-ethyl 3-chloro-4-ethyl-17-{3-[(6-fluoronaphthalen-1-yl)oxy]propyl}-6-methyl-6,7,9,14-tetrahydroindolo[7,1-fg]pyrazolo[3,4-d][2,8]benzoxazacycloundecine-16-carboxylate (see intermediate 58, 365 mg, 548 μmol) in a mixture of THF (9.1 mL) and ethanol (4.5 mL) was added an aqueous solution of lithium hydroxide (4.5 mL, 1.0 M, 4.5 mmol). The resulting mixture was stirred at 40° C. for 40 hours. The mixture was neutralized using 2-molar aqueous hydrochloric acid. After removal of all volatiles, the residue was subjected to flash chromatography (silica gel, dichloromethane/ethanol gradient) to give the title compound (361 mg, 96% yield).

LC-MS (Method 2): Rₜ=0.99 min, MS (ESIpos): m/z=638 [M+H]⁺

¹H-NMR (400 MHz, DMSO-d6) δ [ppm]: 0.816 (4.22), 0.835 (9.56), 0.853 (4.43), 0.884 (1.32), 0.902 (2.62), 0.920 (1.25), 1.035 (5.21), 1.052 (11.42), 1.070 (4.81), 1.232 (0.66), 1.907 (0.86), 2.005 (0.71), 2.022 (1.65), 2.040 (2.24), 2.059 (1.76), 2.065 (3.87), 2.076 (0.71), 2.084 (4.27), 2.177 (1.02), 2.194 (1.48), 2.211 (1.04), 2.331 (1.04), 2.336 (0.48), 2.420 (0.76), 2.438 (0.76), 2.518 (6.10), 2.522 (3.69), 2.673 (1.07), 2.678 (0.48), 3.232 (0.64), 3.251 (1.30), 3.272 (1.37), 3.291 (0.74), 3.405 (0.43), 3.417 (0.53), 3.422 (1.20), 3.435 (1.30), 3.440 (1.25), 3.452 (1.20), 3.839 (1.98), 3.877 (16.00), 4.078 (3.92), 4.108 (0.69), 4.116 (0.76), 4.132 (1.76), 4.147 (1.73), 4.162 (0.71), 4.170 (0.48), 4.344 (0.59), 4.357 (1.09), 4.370 (0.56), 4.703 (1.98), 4.738 (1.91), 5.147 (1.04), 5.185 (1.37), 5.425 (0.76), 5.464 (0.61), 5.759 (0.86), 6.791 (1.27), 6.800 (1.60), 6.806 (2.14), 6.816 (1.63), 6.822 (1.50), 7.064 (0.64), 7.080 (1.27), 7.098 (0.81), 7.141 (0.79), 7.144 (0.79), 7.163 (1.65), 7.172 (2.62), 7.179 (1.20), 7.194 (2.59), 7.210 (2.06), 7.214 (2.01), 7.229 (1.17), 7.233 (1.07), 7.334 (0.86), 7.341 (0.99), 7.357 (1.35), 7.363 (1.50), 7.379 (0.92), 7.385 (0.99), 7.400 (0.59), 7.421 (2.14), 7.438 (4.99), 7.455 (0.56), 7.640 (1.53), 7.646 (1.60), 7.666 (1.55), 7.672 (1.55), 7.724 (2.06), 7.745 (1.88), 8.238 (1.32), 8.253 (1.35), 8.262 (1.35), 8.276 (1.30).

The title compound (355 mg) was separated into enantiomers by preparative chiral HPLC to give enantiomer 1 (156 mg, see Example 58) and enantiomer 2 (170 mg, see Example 59).

Preparative Chiral HPLC Method:

Instrument: Sepiatec: Prep SFC100; column: Chiralpak IG 5 μm 250×30 mm; Eluent A: CO₂, Eluent B: 2-propanol+0.4 Vol-% N-ethylethanamine (99%); isocratic 23% B; Flow 100.0 mL/min Temperature: 40° C.; BPR: 150 bar; MWD @ 220 nm

Analytical Chiral HPLC Method:

Instrument: Agilent: 1260, Aurora SFC-Modul; column: Chiralpak IG 5 μm 100×4.6 mm; Eluent A: CO₂, Eluent B: 2-propanol+0.2 Vol-% N-ethylethanamine (99%); isocratic 23% B; Flow 4.0 mL/min; Temperature: 37.5° C.; BPR: 100 bar; MWD @ 220 nm

### Example 58

### (+)-3-Chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid-N-ethylethanamine Salt (Enantiomer 1)

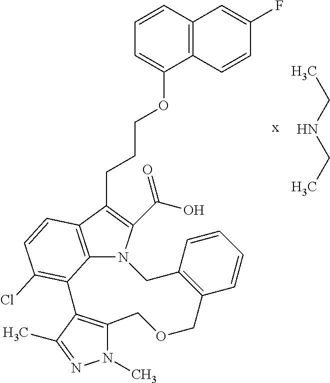

For the preparation of the racemic title compound see Example 57. Separation of enantiomers by preparative chiral HPLC (method see Example 57) gave the title compound (156 mg).

Analytical Chiral HPLC (method see Example 57): Rₜ=2.41 min.

Specific Optical Rotation (Method O1): +57.6° (c=10 mg/mL, DMSO)

LC-MS (Method 2): Rₜ=0.99 min, MS (ESIpos): m/z=638 [M+H]⁺

¹H-NMR (400 MHz, DMSO-d6) δ [ppm]: 0.774 (3.66), 0.793 (8.15), 0.811 (3.73), 1.129 (6.89), 1.147 (16.00), 1.166 (6.96), 1.909 (0.41), 1.928 (0.64), 1.946 (1.06), 1.965 (1.02), 1.988 (1.22), 2.007 (1.17), 2.024 (0.69), 2.043 (0.49), 2.211 (0.94), 2.228 (1.37), 2.246 (0.97), 2.327 (0.92), 2.331 (0.68), 2.518 (3.55), 2.523 (2.36), 2.669 (0.96), 2.673 (0.68), 2.854 (1.86), 2.872 (5.99), 2.890 (5.79), 2.908 (1.70), 3.232 (1.15), 3.250 (1.95), 3.268 (1.17), 3.839 (1.73), 3.874 (1.81), 3.900 (13.38), 4.054 (1.14), 4.084 (1.58), 4.113 (0.59), 4.121 (0.59), 4.137 (1.06), 4.155 (0.84), 4.172 (1.19), 4.183 (1.50), 4.196 (0.71), 4.213 (1.02), 4.665 (1.73), 4.700 (1.67), 4.990 (0.79), 5.028 (0.89), 6.800 (1.19), 6.805 (1.22), 6.816 (1.14), 6.822 (1.22), 6.880 (0.74), 6.899 (1.25), 6.937 (0.68), 6.954 (1.04), 6.973 (0.48), 7.017 (1.39), 7.038 (1.44), 7.071 (0.69), 7.090 (1.27), 7.108 (0.68), 7.200 (1.60), 7.219 (1.22), 7.344 (0.76), 7.351 (0.89), 7.367 (1.20), 7.374 (1.30), 7.389 (1.29), 7.396 (0.96), 7.411 (1.91), 7.428 (4.11), 7.445 (0.51), 7.539 (1.12), 7.560 (1.01), 7.634 (1.40), 7.640 (1.42), 7.660 (1.40), 7.666 (1.37), 8.260 (1.15), 8.275 (1.29), 8.283 (1.20), 8.298 (1.12).

### Example 59

### (−)-3-Chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid-N-ethylethanamine Salt (Enantiomer 2)

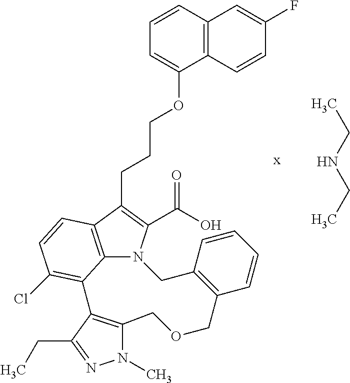

For the preparation of the racemic title compound see Example 57. Separation of enantiomers by preparative chiral HPLC (method see Example 57) gave the title compound (170 mg).

Analytical Chiral HPLC (method see Example 57): Rₜ=5.56 min.

LC-MS (Method 2): Rₜ=0.98 min, MS (ESIpos): m/z=638 [M+H]⁺

Specific Optical Rotation (Method O1): −54.0° (c=10 mg/mL, DMSO)

¹H-NMR (400 MHz, DMSO-d6) δ [ppm]: 0.770 (3.42), 0.789 (7.60), 0.808 (3.48), 1.026 (1.18), 1.042 (1.15), 1.124 (7.03), 1.142 (16.00), 1.160 (7.03), 1.920 (0.57), 1.938 (0.89), 1.957 (0.86), 1.965 (0.48), 1.984 (1.12), 2.003 (1.09), 2.021 (0.67), 2.039 (0.45), 2.212 (0.93), 2.229 (1.34), 2.246 (0.96), 2.327 (1.79), 2.331 (1.31), 2.336 (0.57), 2.518 (6.80), 2.523 (4.57), 2.669 (1.85), 2.673 (1.31), 2.678 (0.57), 2.849 (1.85), 2.867 (5.81), 2.885 (5.65), 2.903 (1.69), 3.228 (1.02), 3.247 (1.76), 3.264 (1.02), 3.838 (1.72), 3.872 (1.79), 3.902 (13.29), 4.055 (1.12), 4.085 (1.56), 4.115 (0.57), 4.122 (0.57), 4.138 (1.02), 4.156 (0.70), 4.175 (1.05), 4.191 (1.53), 4.220 (0.80), 4.662 (1.66), 4.696 (1.60), 4.976 (0.61), 5.014 (0.70), 6.803 (1.15), 6.808 (1.18), 6.819 (1.09), 6.824 (1.21), 6.880 (0.67), 6.900 (1.21), 6.929 (0.64), 6.947 (0.89), 6.966 (0.42), 7.005 (0.99), 7.026 (1.02), 7.067 (0.61), 7.086 (1.18), 7.106 (0.64), 7.202 (1.60), 7.219 (1.18), 7.346 (0.77), 7.353 (0.83), 7.369 (1.15), 7.376 (1.28), 7.391 (1.21), 7.398 (0.93), 7.413 (1.92), 7.425 (2.40), 7.429 (4.50), 7.445 (0.48), 7.525 (0.80), 7.546 (0.73), 7.634 (1.37), 7.641 (1.41), 7.660 (1.37), 7.667 (1.34), 8.263 (1.18), 8.278 (1.21), 8.286 (1.18), 8.301 (1.12).

### Example 60

### (rac)-1-(3-(4-Chloro-3,5-dimethylphenoxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylic Acid

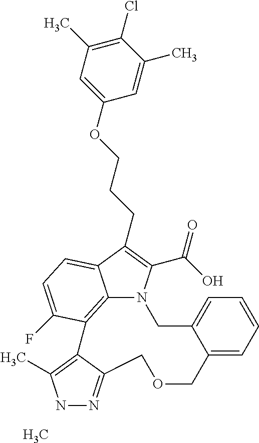

To a stirred solution of (rac)-ethyl 1-(3-(4-chloro-3,5-dimethylphenoxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 67; 59.7 mg, 94.7 μmol, 1.00 eq.) in ethanol (378 μL, 0.25 M) was added a 2.0 M solution of sodium hydroxide in water (118 μL, 237 μmol, 2.50 eq.). The resulting yellow solution was heated to 70° C. for 24 hours, cooled to room temperature and was then acidified with 1.0 M aqueous hydrochloric acid. Celite was added to the mixture, volatiles were removed under reduced pressure, and the residue was subjected to reverse phase column chromatography (10-100% acetonitrile/water with 0.1% formic acid gradient) to give the title compound as a white solid (53.1 mg).

LC-MS (Method 3): Rₜ=4.85 min; MS (ESIpos): m/z=602 [M+H]⁺.

¹H-NMR (300 MHz, DMSO-d) δ [ppm]: 13.26 (s, 1H), 7.69 (dd, 1H), 7.11 (m, 5H), 6.69 (s, 2H), 5.29 (d, 2H), 4.41 (d, 1H), 4.15 (m, 2H), 3.98 (d, 1H), 3.83 (s, 5H), 3.05 (m, 2H), 2.26 (s, 6H), 1.93 (s, 5H).

### Example 61

### (rac)-1-(3-((2,3-Dihydro-1H-inden-4-yl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylic Acid

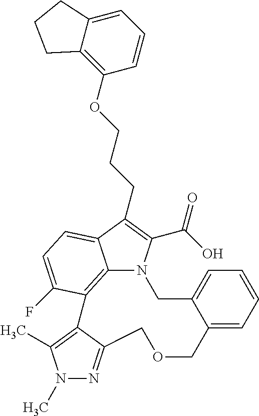 [SMILES: Cc1c2c(nn1C)COCc1ccccc1Cn1c(C(=O)O)c(CCCOc3cccc4c3CCC4)c3ccc(F)c-2c31]

To a stirred solution of (rac)-ethyl 1-(3-((2,3-dihydro-1H-inden-4-yl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 68, 58.6 mg, 96.4 μmol, 1.00 eq.) in ethanol (385 μL, 0.25 M) was added a 2.0 M solution of sodium hydroxide in water (120 μL, 241 μmol, 2.50 eq.). The resulting yellow solution was heated to 70° C. for 24 hours, cooled to room temperature and was then acidified with 1.0 M aqueous hydrochloric acid. Celite was added to the resulting mixture, volatiles were removed under reduced pressure, and the residue was subjected to reverse phase column chromatography (10-100% acetonitrile/water with 0.1% formic acid gradient) to give the title compound as a white solid (50.2 mg).

LC-MS (Method 3): Rₜ=4.45 min, MS (ESIpos): m/z=580 [M+H]⁺.

¹H-NMR (300 MHz, DMSO-d) δ [ppm]: 13.26 (s, 1H), 7.69 (dd, 1H), 7.11 (m, 6H), 6.79 (d, 1H), 6.56 (d, 1H), 5.29 (s, 2H), 4.41 (d, 1H), 4.19 (d, 1H), 4.11 (d, 1H), 3.99 (d, 1H), 3.90 (q, 5H), 3.08 (m, 2H), 2.80 (dt, 4H), 1.96 (m, 7H).

### Example 62

### (rac)-15-Fluoro-13,14-dimethyl-1-(3-((5,6,7,8-tetrahydronaphthalen-1-yl)oxy)propyl)-4,9,11,13-tetrahydrobenzo[3,4]pyrazolo[4′,3′:9,10][1,6]oxaazacycloundecino[8,7,6-hi]indole-2-carboxylic Acid

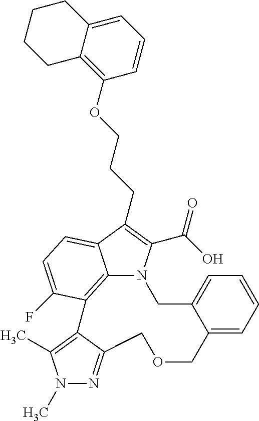 [SMILES: Cc1c2c(nn1C)COCc1ccccc1Cn1c(C(=O)O)c(CCCOc3cccc4c3CCCC4)c3ccc(F)c-2c31]

To a stirred solution of (rac)-ethyl 15-fluoro-13,14-dimethyl-1-(3-((5,6,7,8-tetrahydronaphthalen-1-yl)oxy)propyl)-4,9,11,13-tetrahydrobenzo[3,4]pyrazolo[4′,3′:9,10]-[1,6]oxaazacycloundecino[8,7,6-hi]indole-2-carboxylate (see intermediate 69, 52.7 mg, 84.8 μmol, 1.00 eq.) in ethanol (339 μL, 0.25 M) was added a 2.0 M solution of sodium hydroxide in water (105 μL, 212 μmol, 2.50 eq.). The resulting yellow solution was heated to 70° C. for 24 hours, cooled to room temperature and was then acidified with 1.0 M aqueous hydrochloric acid. Celite was added to the resulting mixture, volatiles were removed under reduced pressure, and the residue was subjected to purified by reverse phase column chromatography (10-100% acetonitrile/water with 0.1% formic acid gradient) to give the title compound as a white solid (43.4 mg).

LC-MS (Method 3): Rₜ=4.71 min, MS (ESIpos): m/z=594 [M+H]⁺.

¹H-NMR (300 MHz, DMSO-d) δ [ppm]: 13.27 (s, 1H), 7.69 (dd, 1H), 7.15 (m, 4H), 7.00 (m, 2H), 6.58 (dd, 2H), 5.29 (s, 2H), 4.41 (d, 1H), 4.19 (d, 1H), 4.11 (d, 1H), 3.99 (d, 1H), 3.83 (s, 5H), 3.09 (m, 2H), 2.66 (m, 2H), 2.57 (t, 2H), 1.94 (s, 5H), 1.69 (q, 4H).

### Example 63

### (rac)-15-Fluoro-1-(3-((4-fluoronaphthalen-1-yl)oxy)propyl)-13,14-dim4,9,11,13-tetrahydrobenzo[3,4]pyrazolo[4′,3′:9,10][1,6]oxaazacycloundecino[8,7,6-hi]indole-2-carboxylic Acid

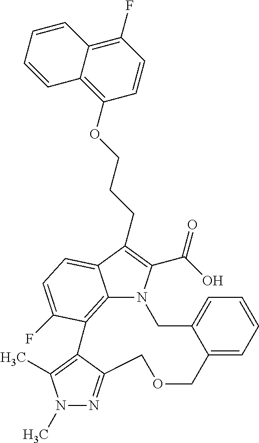 [SMILES: Cc1c2c(nn1C)COCc1ccccc1Cn1c(C(=O)O)c(CCCOc3ccc(F)c4ccccc34)c3ccc(F)c-2c31]

To a stirred solution of (rac)-ethyl 15-fluoro-1-(3-((4-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 70, 148 mg, 233 μmol, 1.00 eq.) in ethanol (931 μL, 0.25 M) was added a 2.0 M solution of sodium hydroxide in water (291 μL, 5842 μmol, 2.50 eq.). The resulting yellow solution was heated to 70° C. for 24 hours, cooled to room temperature and was then acidified with 1.0 M aqueous hydrochloric acid. Celite was added to the resulting mixture, volatiles were removed under reduced pressure, and the residue was subjected to reverse phase column chromatography (10-100% acetonitrile/water with 0.1% formic acid gradient) to give the title compound as an off-white solid (126 mg).

LC-MS (Method 3): Rₜ=4.51 min, MS (ESIpos): m/z=608 [M+H]⁺.

¹H-NMR (300 MHz, DMSO-d) δ [ppm]: 13.34 (s, 1H), 8.22 (dt, 1H), 7.98 (dd, 1H), 7.73 (dd, 1H), 7.63 (m, 2H), 7.18 (m, 5H), 7.02 (t, 1H), 6.74 (dd, 1H), 5.30 (d, 2H), 4.41 (d, 1H), 4.08 (m, 5H), 3.18 (m, 2H), 2.10 (m, 2H), 1.93 (s, 3H).

### Example 64

### (rac)-15-Fluoro-1-(3-((6-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylic Acid

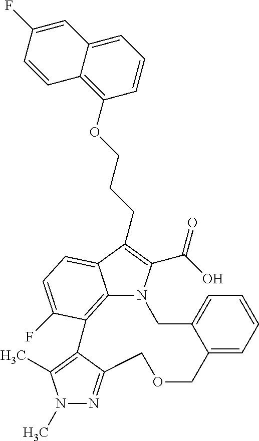 [SMILES: Cc1c2c(nn1C)COCc1ccccc1Cn1c(C(=O)O)c(CCCOc3cccc4cc(F)ccc34)c3ccc(F)c-2c31]

To a stirred solution of (rac)-ethyl 15-fluoro-1-(3-((6-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylate (see intermediate 71, 155 mg, 244 μmol, 1.00 eq.) in ethanol (975 μL, 0.25 M) was added a 2.0 M solution of sodium hydroxide in water (304 μL, 610 μmol, 2.50 eq.). The resulting yellow solution was heated to 70° C. for 22 hours, cooled to room temperature and was then acidified with trifluroacetic acid. Celite was added to the resulting mixture, volatiles were removed under reduced pressure, and the residue was subjected to reverse phase column chromatography (10-100% acetonitrile/water with 0.1% formic acid gradient) to give the title compound as a yellow solid (141 mg).

LC-MS (Method 3): Rₜ=4.45 min; MS (ESIpos): m/z=608 [M+H]⁺.

¹H-NMR (300 MHz, DMSO-d) δ [ppm]: 13.35 (s, 1H), 8.23 (dd, 1H), 7.74 (dd, 1H), 7.65 (dd, 1H), 7.40 (m, 3H), 7.17 (m, 4H), 7.02 (t, 1H), 6.78 (dd, 1H), 5.30 (d, 2H), 4.41 (d, 1H), 4.09 (m, 5H), 3.83 (s, 3H), 3.19 (m, 2H), 2.09 (d, 2H), 1.93 (s, 3H).

The title compound (114 mg) was separated into enantiomers by preparative chiral HPLC to give enantiomer 1 (25 mg, see Example 65) and enantiomer 2 (47 mg, see Example 66).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; isocratic 50% A+50% B; Flow 50.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 65

### (−)-3-fluoro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine Salt (Enantiomer 1)

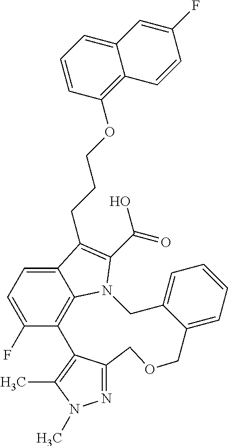 [SMILES: Cc1c2c(nn1C)COCc1ccccc1Cn1c(C(=O)O)c(CCCOc3cccc4cc(F)ccc34)c3ccc(F)c-2c31]

For the preparation of the racemic title compound see Example 64. Separation of enantiomers by preparative chiral HPLC (method see Example 64) gave the title compound (25 mg).

Analytical Chiral HPLC (method see Example 64): Rₜ=2.26 min.

LC-MS (Method 1): Rₜ=1.50 min; MS (ESIpos): m/z=608 [M+H]⁺

Specific Optical Rotation (Method O1): −24.4° (c=10 mg/mL, DMSO)

¹H-NMR (400 MHz, DMSO-d6) δ [ppm]: 1.135 (7.42), 1.154 (16.00), 1.172 (7.57), 1.874 (13.90), 2.113 (1.22), 2.130 (1.80), 2.147 (1.27), 2.165 (0.43), 2.323 (0.70), 2.327 (0.95), 2.331 (0.68), 2.522 (2.90), 2.665 (0.75), 2.669 (0.98), 2.673 (0.72), 2.841 (2.13), 2.858 (6.37), 2.877 (6.30), 2.895 (1.97), 3.134 (0.62), 3.147 (0.82), 3.166 (1.38), 3.185 (1.00), 3.195 (1.37), 3.213 (1.02), 3.228 (0.87), 3.828 (15.50), 4.042 (1.48), 4.066 (2.83), 4.083 (1.30), 4.099 (0.68), 4.113 (2.38), 4.137 (2.20), 4.153 (0.80), 4.160 (0.85), 4.175 (2.07), 4.207 (2.27), 4.368 (2.43), 4.401 (1.92), 5.064 (1.00), 5.101 (1.15), 5.628 (0.72), 5.663 (0.65), 6.771 (1.33), 6.778 (1.37), 6.786 (1.28), 6.792 (1.40), 6.838 (0.88), 6.861 (1.57), 6.884 (0.92), 7.069 (0.68), 7.087 (5.35), 7.093 (8.18), 7.340 (0.82), 7.346 (0.93), 7.362 (1.53), 7.369 (1.80), 7.384 (2.02), 7.391 (2.03), 7.409 (2.65), 7.418 (2.98), 7.424 (5.68), 7.438 (0.50), 7.515 (0.78), 7.528 (0.92), 7.536 (0.90), 7.550 (0.75), 7.626 (1.57), 7.633 (1.60), 7.652 (1.58), 7.659 (1.55), 8.231 (1.37), 8.246 (1.45), 8.254 (1.42), 8.269 (1.32).

### Example 66

### (+)-3-Fluoro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine Salt (Enantiomer 2)

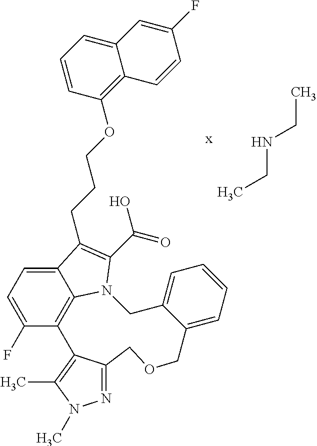

For the preparation of the racemic title compound see Example 64. Separation of enantiomers by preparative chiral HPLC (method see Example 64) gave the title compound (47 mg).

Analytical Chiral HPLC (method see Example 64): Rₜ=5.42 min.

LC-MS (Method 1): Rₜ=1.50 min, MS (ESIpos): m/z=608 [M+H]⁺

Specific Optical Rotation (Method O1): +25.8° (c=10 mg/mL, DMSO)

¹H-NMR (400 MHz, DMSO-d6) δ [ppm]: 0.797 (0.48), 0.814 (0.54), 0.821 (0.54), 0.904 (0.50), 1.132 (7.21), 1.150 (16.00), 1.168 (7.40), 1.230 (0.45), 1.873 (13.48), 2.112 (1.14), 2.130 (1.69), 2.147 (1.18), 2.165 (0.41), 2.327 (1.10), 2.331 (0.81), 2.518 (4.73), 2.523 (2.91), 2.669 (1.12), 2.673 (0.83), 2.836 (1.98), 2.854 (5.95), 2.872 (5.71), 2.890 (1.84), 3.133 (0.56), 3.146 (0.76), 3.166 (1.30), 3.176 (0.87), 3.193 (1.30), 3.211 (0.91), 3.226 (0.76), 3.245 (0.66), 3.828 (15.15), 4.042 (1.41), 4.068 (2.62), 4.083 (1.22), 4.099 (0.62), 4.114 (2.25), 4.139 (2.09), 4.153 (0.74), 4.161 (0.79), 4.175 (1.94), 4.207 (2.17), 4.368 (2.36), 4.400 (1.84), 5.059 (0.89), 5.095 (1.03), 5.634 (0.62), 5.670 (0.56), 6.772 (1.28), 6.779 (1.28), 6.787 (1.18), 6.794 (1.34), 6.834 (0.79), 6.857 (1.41), 6.880 (0.83), 7.068 (0.66), 7.086 (4.86), 7.093 (7.67), 7.340 (0.79), 7.347 (0.89), 7.362 (1.43), 7.369 (1.67), 7.385 (1.80), 7.391 (1.98), 7.410 (2.60), 7.418 (2.81), 7.424 (5.62), 7.439 (0.46), 7.511 (0.70), 7.524 (0.81), 7.531 (0.81), 7.545 (0.68), 7.627 (1.51), 7.633 (1.57), 7.653 (1.53), 7.659 (1.51), 8.232 (1.34), 8.246 (1.38), 8.255 (1.36), 8.270 (1.26).

### Example 67

### (rac)-15-Fluoro-13,14-dimethyl-1-(3-(naphthalen-1-yloxy)propyl)-4,9,11,13-tetrahydrobenzo[3,4]pyrazolo[4′,3′:9,10][1,6]oxaazacycloundecino[8,7,6-hi]indole-2-carboxylic Acid

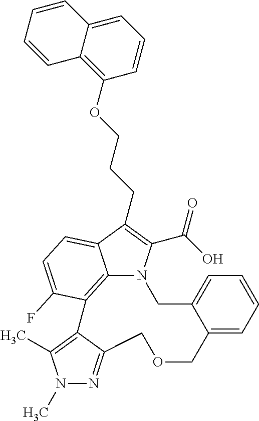 [SMILES: Cc1c2c(nn1C)COCc1ccccc1Cn1c(C(=O)O)c(CCCOc3cccc4ccccc34)c3ccc(F)c-2c31]

To a stirred solution of (rac)-ethyl 15-fluoro-13,14-dimethyl-1-(3-(naphthalen-1-yloxy)propyl)-4,9,11,13-tetrahydrobenzo[3,4]pyrazolo[4′,3′:9,10][1,6]oxaazacycloundecino[8,7,6-hi]indole-2-carboxylate (see intermediate 72, 150 mg, 243 μmol, 1.00 eq.) in ethanol (971 μL, 0.25 M) was added a 2.0 M solution of sodium hydroxide in water (303 μL, 607 μmol, 2.50 eq.). The resulting yellow solution was heated to 70° C. for 22 hours, cooled to room temperature and was then acidified with trifluroacetic acid. Celite was added to the resulting mixture, volatiles were removed under reduced pressure, and the residue was subjected to reverse phase column chromatography (10-100% acetonitrile/water with 0.1% formic acid gradient) to give the title compound as a light yellow solid (129 mg).

LC-MS (Method 3): Rₜ=4.38 min, MS (ESIpos): m/z=590 [M+H]⁺.

¹H-NMR (300 MHz, DMSO-d) δ [ppm]: 13.35 (s, 1H), 8.19 (m, 1H), 7.86 (m, 1H), 7.74 (dd, 1H), 7.46 (m, 4H), 7.17 (m, 4H), 7.02 (t, 1H), 6.80 (dd, 1H), 5.30 (d, 2H), 4.41 (d, 1H), 4.08 (m, 5H), 3.83 (s, 3H), 3.18 (m, 2H), 2.10 (m, 2H), 1.93 (s, 3H).

The title compound (121 mg) was separated into enantiomers by preparative chiral HPLC to give enantiomer 1 (40 mg, see Example 68) and enantiomer 2 (68 mg, see Example 69).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; isocratic 50% A+50% B; Flow 50.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 68

### (−)-3-Fluoro-4,5-dimethyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine Salt (Enantiomer 1)

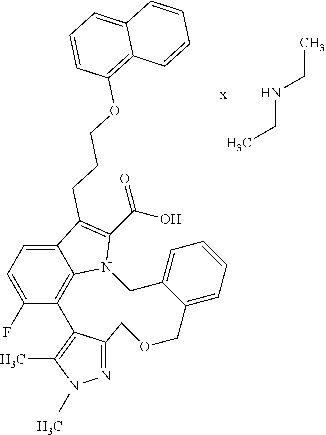

For the preparation of the racemic title compound see Example 67. Separation of enantiomers by preparative chiral HPLC (method see Example 67) gave the title compound (40 mg).

Analytical Chiral HPLC (method see Example 67): Rₜ=2.34 min.

LC-MS (Method 1): Rₜ=1.48 min; MS (ESIpos): m/z=590 [M+H]⁺

Specific Optical Rotation (Method O1): −25.5° (c=10 mg/mL, DMSO)

¹H-NMR (400 MHz, DMSO-d6) δ [ppm]: 1.137 (7.19), 1.155 (16.00), 1.173 (7.41), 1.876 (13.95), 2.119 (1.23), 2.136 (1.85), 2.153 (1.30), 2.170 (0.42), 2.331 (0.77), 2.518 (5.00), 2.522 (3.06), 2.669 (1.08), 2.673 (0.79), 2.841 (2.09), 2.859 (6.30), 2.877 (6.06), 2.895 (1.94), 3.136 (0.66), 3.150 (0.84), 3.169 (1.37), 3.186 (1.15), 3.202 (1.41), 3.219 (1.10), 3.235 (1.03), 3.828 (15.56), 4.044 (1.48), 4.068 (2.76), 4.084 (1.32), 4.101 (0.70), 4.113 (2.40), 4.138 (2.12), 4.153 (0.79), 4.160 (0.86), 4.173 (2.01), 4.205 (2.25), 4.368 (2.45), 4.400 (1.94), 5.066 (0.95), 5.102 (1.10), 5.627 (0.66), 5.662 (0.60), 6.798 (1.98), 6.816 (2.12), 6.836 (0.88), 6.858 (1.54), 6.880 (0.88), 7.073 (0.57), 7.089 (5.66), 7.095 (7.54), 7.346 (1.32), 7.366 (2.75), 7.385 (3.00), 7.427 (2.86), 7.448 (1.61), 7.464 (0.53), 7.467 (0.66), 7.481 (1.56), 7.485 (1.45), 7.495 (1.78), 7.500 (2.76), 7.505 (1.83), 7.515 (1.74), 7.519 (2.18), 7.532 (1.34), 7.536 (1.39), 7.543 (0.90), 7.557 (0.71), 7.841 (1.72), 7.859 (1.72), 7.864 (1.45), 8.195 (1.48), 8.199 (1.54), 8.218 (1.45).

### Example 69

### (+)-3-Fluoro-4,5-dimethyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine Salt (Enantiomer 2)

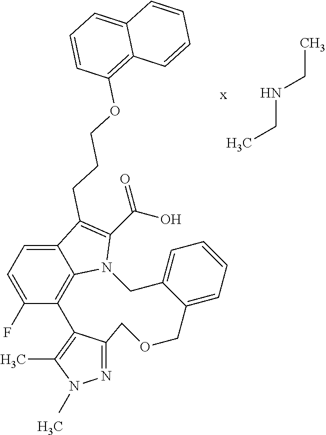

For the preparation of the racemic title compound see Example 67. Separation of enantiomers by preparative chiral HPLC (method see Example 67) gave the title compound (68 mg).

Analytical Chiral HPLC (method see Example 67): Rₜ=6.03 min.

LC-MS (Method 1): Rₜ=1.48 min, MS (ESIpos): m/z=590 [M+H]⁺

Specific Optical Rotation (Method O1): +21.2° (c=10 mg/mL, DMSO)

¹H-NMR (400 MHz, DMSO-d6) δ [ppm]: 0.797 (0.50), 0.814 (0.55), 0.821 (0.52), 0.904 (0.55), 1.127 (7.52), 1.145 (16.00), 1.163 (7.73), 1.872 (13.42), 2.118 (1.20), 2.136 (1.77), 2.153 (1.25), 2.170 (0.44), 2.327 (1.49), 2.331 (1.07), 2.518 (7.10), 2.522 (4.31), 2.669 (1.54), 2.673 (1.12), 2.825 (2.04), 2.843 (5.95), 2.861 (5.82), 2.879 (1.88), 3.136 (0.63), 3.150 (0.84), 3.169 (1.38), 3.187 (1.02), 3.197 (1.44), 3.215 (1.10), 3.231 (1.02), 3.828 (14.90), 4.047 (1.31), 4.060 (0.97), 4.073 (2.51), 4.084 (1.33), 4.101 (0.63), 4.117 (2.32), 4.141 (1.98), 4.155 (0.78), 4.163 (0.89), 4.174 (1.91), 4.205 (2.22), 4.366 (2.40), 4.398 (1.85), 5.050 (0.91), 5.085 (1.02), 5.649 (0.63), 5.684 (0.57), 6.800 (1.88), 6.819 (2.30), 6.846 (1.46), 6.868 (0.86), 7.068 (0.68), 7.086 (4.80), 7.091 (7.44), 7.346 (1.23), 7.367 (2.51), 7.386 (2.66), 7.427 (2.77), 7.448 (1.57), 7.468 (0.65), 7.481 (1.54), 7.485 (1.41), 7.495 (1.77), 7.501 (2.92), 7.505 (2.30), 7.515 (1.96), 7.519 (2.35), 7.531 (1.23), 7.542 (0.78), 7.841 (1.64), 7.859 (1.62), 7.864 (1.38), 8.196 (1.44), 8.201 (1.46), 8.219 (1.38).

### Example 70

### (rac)-14-Ethyl-15-fluoro-13-methyl-1-(3-(naphthalen-1-yloxy)propyl)-4,9,11,13-tetrahydrobenzo[3,4]pyrazolo[4′,3′:9,10][1]oxa[6]azacycloundecino[8,7,6-hi]indole-2-carboxylic Acid

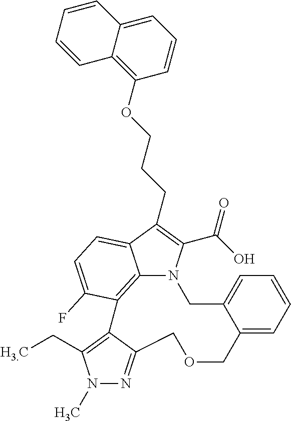

To a stirred solution of (rac)-ethyl 14-ethyl-15-fluoro-13-methyl-1-(3-(naphthalen-1-yloxy)propyl)-4,9,11,13-tetrahydrobenzo[3,4]pyrazolo[4′,3′:9,10][1]oxa[6]azacycloundecino-[8,7,6-hi]indole-2-carboxylate (see intermediate 77, 137 mg, 217 μmol, 1.00 eq.) in ethanol (867 μL, 0.25 M) was added a 2.0 M solution of sodium hydroxide in water (270 μL, 250 μmol, 2.50 eq.). The resulting yellow solution was heated to 70° C. for 2 days and then cooled to room temperature. The mixture was diluted with dimethyl sulfoxide, acidified with trifluoroacetic acid and was then purified by reverse phase column chromatography (10-100% acetonitrile/water with 0.1% formic acid gradient) to give the title compound as an off-white solid (104 mg).

LC-MS (Method 3): Rₜ=4.55 min, MS (ESIpos): m/z=604 [M+H]⁺.

¹H-NMR (300 MHz, DMSO-d) δ [ppm]: 13.36 (s, 1H), 8.20 (m, 1H), 7.85 (m, 1H), 7.74 (dd, 1H), 7.46 (m, 4H), 7.15 (m, 5H), 6.78 (dd, 1H), 5.30 (m, 2H), 4.42 (d, 1H), 4.08 (m, 5H), 3.19 (ddq, 2H), 2.31 (m, 2H), 2.09 (m, 2H), 0.92 (t, 3H).

The title compound (83 mg) was separated into enantiomers by preparative chiral HPLC to give enantiomer 1 (39 mg, see Example 71) and enantiomer 2 (36 mg, see Example 72).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; isocratic 48% B; Flow 45.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 71

### (−)-4-Ethyl-3-fluoro-5-methyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine Salt (Enantiomer 1)

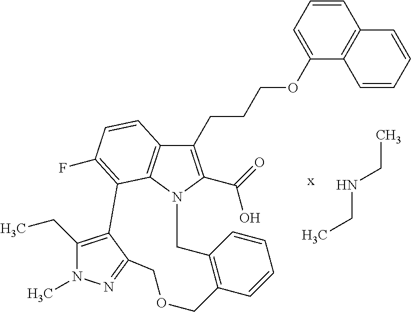

For the preparation of the racemic title compound see Example 70. Separation of enantiomers by preparative chiral HPLC (method see Example 70) gave the title compound (39 mg).

Analytical Chiral HPLC (method see Example 70): Rₜ=2.14 min.

LC-MS (Method 1): Rₜ=1.52 min; MS (ESIpos): m/z=604 [M+H]⁺

Specific Optical Rotation (Method O1): −42.8° (c=10 mg/mL, DMSO)

¹H-NMR (400 MHz, DMSO-d6) δ [ppm]: 0.863 (2.56), 0.882 (5.68), 0.901 (2.60), 1.128 (7.15), 1.146 (16.00), 1.164 (7.23), 2.118 (0.94), 2.135 (1.42), 2.152 (1.01), 2.170 (0.39), 2.208 (0.57), 2.226 (0.96), 2.245 (0.90), 2.256 (0.85), 2.275 (0.87), 2.293 (0.55), 2.327 (1.29), 2.331 (0.96), 2.518 (5.07), 2.523 (3.41), 2.673 (0.94), 2.678 (0.42), 2.825 (1.84), 2.843 (5.64), 2.861 (5.44), 2.879 (1.70), 3.161 (0.87), 3.174 (1.46), 3.179 (1.46), 3.193 (0.92), 3.857 (13.68), 4.052 (1.46), 4.069 (1.22), 4.077 (1.88), 4.109 (0.55), 4.123 (2.25), 4.148 (1.51), 4.171 (1.55), 4.203 (1.75), 4.370 (2.01), 4.403 (1.62), 5.069 (0.70), 5.104 (0.79), 5.639 (0.46), 5.673 (0.44), 6.778 (1.55), 6.797 (1.68), 6.824 (0.68), 6.847 (1.18), 6.870 (0.68), 7.088 (5.55), 7.095 (4.55), 7.111 (0.48), 7.340 (1.09), 7.360 (2.10), 7.379 (1.66), 7.426 (2.93), 7.446 (1.46), 7.462 (0.48), 7.466 (0.59), 7.479 (1.36), 7.483 (1.22), 7.495 (1.51), 7.500 (2.16), 7.504 (1.64), 7.514 (1.64), 7.518 (1.77), 7.531 (1.25), 7.545 (0.57), 7.840 (1.36), 7.845 (0.85), 7.858 (1.46), 7.863 (1.16), 8.194 (1.22), 8.198 (1.22), 8.217 (1.16).

### Example 72

### (+)-4-Ethyl-3-fluoro-5-methyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine Salt (Enantiomer 2)

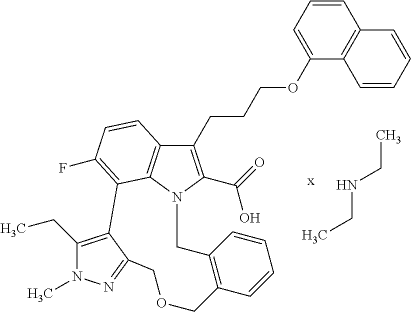

For the preparation of the racemic title compound see Example 70. Separation of enantiomers by preparative chiral HPLC (method see Example 70) gave the title compound (36 mg).

Analytical Chiral HPLC (method see Example 70): Rₜ=5.23 min.

LC-MS (Method 1): Rₜ=1.52 min, MS (ESIpos): m/z=604 [M+H]⁺

Specific Optical Rotation (Method O1): +40.6° (c=10 mg/mL, DMSO)

¹H-NMR (400 MHz, DMSO-d6) δ [ppm]: 0.864 (2.67), 0.883 (5.96), 0.901 (2.72), 1.128 (7.57), 1.146 (16.00), 1.165 (7.80), 2.118 (1.02), 2.135 (1.54), 2.153 (1.09), 2.170 (0.43), 2.208 (0.61), 2.227 (1.02), 2.245 (0.93), 2.257 (0.93), 2.276 (0.93), 2.294 (0.59), 2.322 (0.96), 2.327 (1.28), 2.331 (0.98), 2.518 (5.15), 2.523 (3.39), 2.669 (1.28), 2.673 (0.93), 2.825 (1.89), 2.843 (5.67), 2.862 (5.46), 2.879 (1.74), 3.162 (0.89), 3.174 (1.52), 3.179 (1.50), 3.194 (0.93), 3.857 (14.20), 4.053 (1.54), 4.069 (1.30), 4.078 (1.98), 4.110 (0.59), 4.123 (2.41), 4.148 (1.59), 4.171 (1.61), 4.203 (1.85), 4.371 (2.13), 4.403 (1.67), 5.069 (0.74), 5.106 (0.83), 5.638 (0.48), 5.673 (0.46), 6.778 (1.65), 6.796 (1.76), 6.825 (0.72), 6.848 (1.24), 6.870 (0.72), 7.089 (5.74), 7.096 (4.85), 7.112 (0.50), 7.339 (1.15), 7.360 (2.20), 7.379 (1.70), 7.426 (3.15), 7.446 (1.57), 7.462 (0.50), 7.466 (0.63), 7.479 (1.41), 7.483 (1.30), 7.495 (1.63), 7.500 (2.28), 7.504 (1.76), 7.514 (1.78), 7.518 (1.87), 7.524 (0.87), 7.531 (1.30), 7.546 (0.59), 7.840 (1.48), 7.845 (0.91), 7.858 (1.52), 7.863 (1.22), 8.194 (1.28), 8.198 (1.33), 8.217 (1.22).

### Example 73

### (rac)-14-Ethyl-15-fluoro-1-(3-((6-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylic Acid

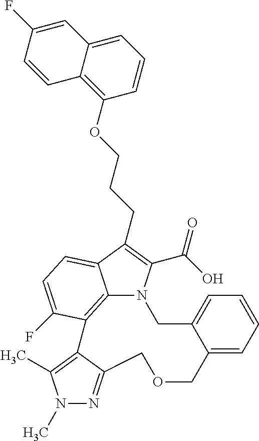 [SMILES: Cc1c2c(nn1C)COCc1ccccc1Cn1c(C(=O)O)c(CCCOc3cccc4cc(F)ccc34)c3ccc(F)c-2c31]

To a stirred solution of (rac)-ethyl 14-ethyl-15-fluoro-1-(3-((6-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylate (see intermediate 78, 148 mg, 227 μmol, 1.00 eq.) in ethanol (910 μL, 0.25 M) was added a 2.0 M solution of sodium hydroxide in water (284 μL, 569 μmol, 2.50 eq.). The resulting yellow solution was heated to 70° C. for 2 days and then cooled to room temperature. The mixture was diluted with dimethyl sulfoxide, acidified with trifluoroacetic acid and was then purified by reverse phase column chromatography (10-100% acetonitrile/water with 0.1% formic acid gradient) to give the title compound as an off-white solid (110 mg).

LC-MS (Method 3): Rₜ=4.63 min, MS (ESIpos): m/z=622 [M+H]⁺.

¹H-NMR (300 MHz, DMSO-d) δ [ppm]: 13.40 (s, 1H), 8.24 (dd, 1H), 7.69 (ddd, 2H), 7.39 (m, 3H), 7.17 (m, 4H), 7.02 (t, 1H), 6.76 (dd, 1H), 5.31 (m, 2H), 4.42 (d, 1H), 4.08 (m, 5H), 3.86 (s, 3H), 3.16 (m, 2H), 2.31 (m, 2H), 2.10 (m, 2H), 0.91 (t, 3H).

The title compound (87 mg) was separated into enantiomers by preparative chiral HPLC to give enantiomer 1 (32 mg, see Example 74) and enantiomer 2 (31 mg, see Example 75).

Preparative Chiral HPLC Method:

Instrument: Labomatic HD5000, Labocord-5000; Gilson GX-241, Labcol Vario 4000, column: Chiralpak IG 5μ 250×30 mm; Eluent A: hexane+0.1 Vol-% N-ethylethanamine (99%); Eluent B: ethanol; isokratic 60% A+40% B; Flow 50.0 mL/min; UV 254 nm

Analytical Chiral HPLC Method:

Instrument: Agilent HPLC 1260; column: Chiralpak IG 3μ 100×4.6 mm; Eluent A: hexane+0.1 Vol-% N-ethylethanamine (99%); Eluent B: ethanol; Gradient: 20-50% B in 7 min; Flow 1.4 mL/min; Temperature: 25° C.; DAD 254 nm

### Example 74

### (−)-4-Ethyl-3-fluoro-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic acid-N-ethylethanamine Salt (Enantiomer 1)

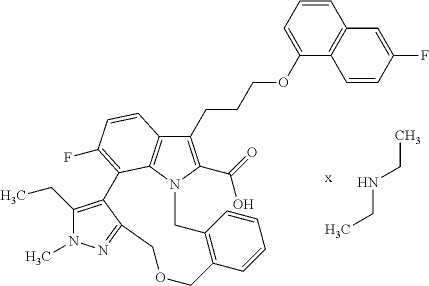

For the preparation of the racemic title compound see Example 73. Separation of enantiomers by preparative chiral HPLC (method see Example 73) gave the title compound (32 mg).

Analytical Chiral HPLC (method see Example 73): Rₜ=2.16 min.

LC-MS (Method 1): Rₜ=1.53 min; MS (ESIpos): m/z=622 [M+H]⁺

Specific Optical Rotation (Method O1): −39.0° (c=10 mg/mL, DMSO)

¹H-NMR (400 MHz, DMSO-d6) δ [ppm]: 0.814 (0.43), 0.821 (0.42), 0.862 (3.17), 0.881 (6.95), 0.900 (3.27), 1.134 (6.90), 1.151 (14.98), 1.170 (7.20), 1.229 (0.47), 2.112 (1.14), 2.130 (1.71), 2.147 (1.23), 2.164 (0.45), 2.188 (0.45), 2.207 (0.74), 2.225 (1.21), 2.244 (1.12), 2.256 (1.09), 2.275 (1.11), 2.293 (0.69), 2.312 (0.43), 2.331 (0.81), 2.518 (4.58), 2.522 (2.94), 2.673 (0.80), 2.834 (1.95), 2.853 (5.93), 2.870 (5.73), 2.889 (1.85), 3.156 (1.14), 3.170 (1.92), 3.190 (1.26), 3.856 (16.00), 4.049 (1.78), 4.074 (2.40), 4.108 (0.66), 4.120 (2.73), 4.146 (1.83), 4.172 (1.89), 4.204 (2.18), 4.372 (2.42), 4.404 (1.94), 5.075 (0.97), 5.110 (1.12), 5.629 (0.71), 5.665 (0.64), 6.750 (1.35), 6.755 (1.38), 6.766 (1.31), 6.772 (1.44), 6.832 (0.90), 6.854 (1.59), 6.877 (0.93), 7.087 (6.18), 7.095 (6.56), 7.110 (0.50), 7.338 (0.81), 7.344 (0.95), 7.360 (1.42), 7.367 (1.54), 7.382 (1.38), 7.389 (1.04), 7.403 (2.51), 7.419 (6.12), 7.435 (1.18), 7.509 (0.80), 7.523 (0.92), 7.530 (0.90), 7.544 (0.74), 7.625 (1.57), 7.631 (1.61), 7.651 (1.57), 7.657 (1.56), 8.229 (1.35), 8.244 (1.42), 8.253 (1.40), 8.267 (1.31).

### Example 75

### (+)-4-Ethyl-3-fluoro-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic acid-N-ethylethanamine Salt (Enantiomer 2)

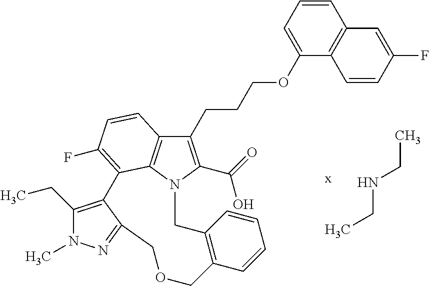

For the preparation of the racemic title compound see Example 73. Separation of enantiomers by preparative chiral HPLC (method see Example 73) gave the title compound (31 mg).

Analytical Chiral HPLC (method see Example 73): Rₜ=5.08 min.

LC-MS (Method 1): Rₜ=1.53 min, MS (ESIpos): m/z=622 [M+H]⁺

Specific Optical Rotation (Method O1): +42.0° (c=10 mg/mL, DMSO)

¹H-NMR (400 MHz, DMSO-d6) δ [ppm]: 0.798 (0.66), 0.814 (0.76), 0.821 (0.72), 0.840 (0.41), 0.862 (3.03), 0.881 (6.66), 0.900 (3.03), 0.904 (1.57), 0.922 (0.42), 1.132 (6.35), 1.150 (13.99), 1.168 (6.63), 1.231 (0.48), 1.259 (0.46), 2.112 (1.00), 2.130 (1.51), 2.147 (1.07), 2.188 (0.42), 2.207 (0.66), 2.225 (1.09), 2.244 (1.00), 2.257 (0.96), 2.275 (0.98), 2.294 (0.63), 2.332 (0.83), 2.518 (4.50), 2.522 (3.10), 2.673 (0.79), 2.833 (1.70), 2.852 (5.09), 2.869 (5.02), 2.888 (1.59), 3.156 (0.96), 3.170 (1.66), 3.189 (1.01), 3.857 (16.00), 4.048 (1.55), 4.074 (2.18), 4.108 (0.59), 4.121 (2.45), 4.145 (1.66), 4.171 (1.73), 4.204 (2.05), 4.372 (2.31), 4.404 (1.81), 5.074 (0.81), 5.111 (0.94), 5.627 (0.55), 5.663 (0.52), 6.750 (1.25), 6.756 (1.29), 6.767 (1.22), 6.772 (1.29), 6.832 (0.76), 6.855 (1.35), 6.877 (0.79), 7.088 (5.72), 7.095 (5.72), 7.110 (0.48), 7.338 (0.83), 7.344 (0.89), 7.360 (1.31), 7.367 (1.40), 7.382 (1.33), 7.389 (1.01), 7.403 (2.40), 7.419 (5.61), 7.435 (0.92), 7.510 (0.66), 7.524 (0.78), 7.531 (0.76), 7.544 (0.63), 7.625 (1.46), 7.632 (1.51), 7.651 (1.48), 7.658 (1.46), 8.229 (1.27), 8.244 (1.35), 8.253 (1.27), 8.267 (1.24).

### Example 76

### (rac)-14-Ethyl-15-fluoro-1-(3-((4-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylic Acid

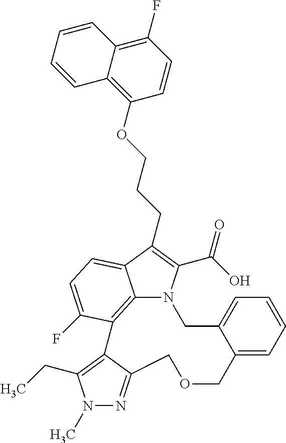 [SMILES: CCc1c2c(nn1C)COCc1ccccc1Cn1c(C(=O)O)c(CCCOc3ccc(F)c4ccccc34)c3ccc(F)c-2c31]

To a stirred solution of (rac)-ethyl 14-ethyl-15-fluoro-1-(3-((4-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylate (see intermediate 79, 122 mg, 188 μmol, 1.00 eq.) in ethanol (750 μL, 0.25 M) was added a 2.0 M solution of sodium hydroxide in water (234 μL, 469 μmol, 2.50 eq.). The resulting yellow solution was heated to 70° C. for 2 days and then cooled to room temperature. The mixture was diluted with dimethyl sulfoxide, acidified with trifluoroacetic acid and was then purified by reverse phase column chromatography (10-100% acetonitrile/water with 0.1% formic acid gradient) to give the title compound as an off-white solid (94.7 mg).

LC-MS (Method 3): Rₜ=4.66 min, MS (ESIpos): m/z=622 [M+H]⁺.

¹H-NMR (300 MHz, DMSO-d) δ [ppm]: 13.35 (s, 1H), 8.22 (m, 1H), 7.98 (dd, 1H), 7.73 (dd, 1H), 7.64 (m, 2H), 7.17 (m, 5H), 7.02 (t, 1H), 6.72 (dd, 1H), 5.30 (m, 2H), 4.41 (d, 1H), 4.07 (m, 5H), 3.86 (s, 3H), 3.17 (m, 2H), 2.30 (m, 2H), 2.10 (m, 2H), 0.91 (t, 3H).

### Example 77

### (rac)-14-Ethyl-15-fluoro-13-methyl-1-(3-((5,6,7,8-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylic Acid

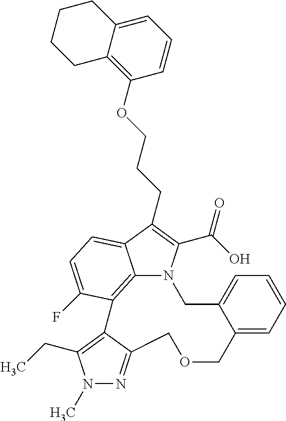 [SMILES: CCc1c2c(nn1C)COCc1ccccc1Cn1c(C(=O)O)c(CCCOc3cccc4c3CCCC4)c3ccc(F)c-2c31]

To a stirred solution of (rac)-ethyl 14-ethyl-15-fluoro-13-methyl-1-(3-((5,6,7,8-tetrahydronaphthalen-1-yl)oxy)propyl)-4,9,11,13-tetrahydrobenzo[3,4]pyrazolo[4′,3′:9,10]-[1]oxa[6]azacycloundecino[8,7,6-hu]indole-2-carboxylate (see intermediate 80, 61.0 mg, 95.9 μmol, 1.00 eq.) in ethanol (383 μL, 0.25 M) was added a 2.0 M solution of sodium hydroxide in water (119 μL, 240 μmol, 2.50 eq.). The resulting yellow solution was heated to 70° C. for 2 days and then cooled to room temperature. The mixture was diluted with dimethyl sulfoxide, acidified with trifluoroacetic acid and was then purified by reverse phase column chromatography (10-100% acetonitrile/water with 0.1% formic acid gradient) to give the title compound as a white solid (38.3 mg).

LC-MS (Method 3): Rₜ=4.89 min, (Method 3)MS (ESIpos): m/z=608 [M+H]⁺.

¹H-NMR (300 MHz, DMSO-d) δ [ppm]: 13.28 (s, 1H), 7.69 (dd, 1H), 7.12 (m, 6H), 6.62 (d, 1H), 6.52 (d, 1H), 5.31 (m, 2H), 4.42 (d, 1H), 4.15 (m, 2H), 3.99 (d, 1H), 3.86 (m, 5H), 3.08 (m, 2H), 2.67 (t, 2H), 2.57 (m, 2H), 2.32 (m, 2H), 1.96 (m, 2H), 1.68 (m, 4H), 0.92 (t, 3H).

### Example 78

### (rac)-1-(3-((2,3-Dihydro-1H-inden-4-yl)oxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylic

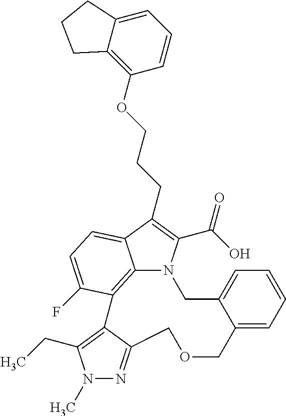 [SMILES: CCc1c2c(nn1C)COCc1ccccc1Cn1c(C(=O)O)c(CCCOc3cccc4c3CCC4)c3ccc(F)c-2c31]

To a stirred solution of (rac)-ethyl 1-(3-((2,3-dihydro-1H-inden-4-yl)oxy)propyl)-14-ethyl-15-fluoro-13-methyl-4,9,11,13-tetrahydrobenzo[3,4]pyrazolo[4′,3′:9,10][1]oxa[6]azacycloundecino-[8,7,6-hi]indole-2-carboxylate (see intermediate 82, 51.0 mg, 82.0 μmol, 1.00 eq.) in ethanol (328 μL, 0.25 M) was added a 2.0 M solution of sodium hydroxide in water (102 μL, 205 μmol, 2.50 eq.). The resulting yellow solution was heated to 70° C. for 2 days and then cooled to room temperature. The mixture was diluted with dimethyl sulfoxide, acidified with trifluoroacetic acid and was then purified by reverse phase column chromatography (10-100% acetonitrile/water with 0.1% formic acid gradient) to give 1 the title compound as as a white solid (25.4 mg).

LC-MS (Method 3): Rₜ=4.63 min, MS (ESIpos): m/z=594 [M+H]⁺.

¹H-NMR (300 MHz, DMSO-d) δ [ppm]: 13.26 (s, 1H), 7.69 (dd, 1H), 7.13 (m, 6H), 6.79 (d, 1H), 6.53 (d, 1H), 5.31 (m, 2H), 4.42 (d, 1H), 4.14 (m, 2H), 3.93 (m, 6H), 3.07 (m, 2H), 2.80 (m, 4H), 2.32 (m, 2H), 1.99 (m, 4H), 0.92 (t, 3H).

### Example 79

### (rac)-1-(3-(4-Chloro-3,5-dimethylphenoxy)propyl)-14-15-fluoro-13-methyl-4,9,11,13-tetrahydrobenzo[3,4]pyrazolo[4′,3′:9,10][1]oxa[6]azacycloundecino[8,7,6-hi]indole-2-carboxylic Acid

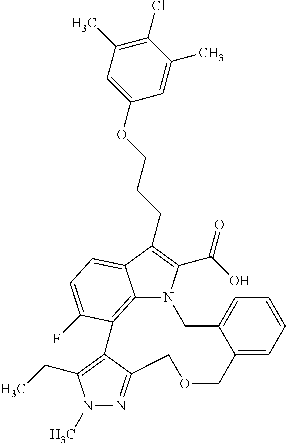 [SMILES: CCc1c2c(nn1C)COCc1ccccc1Cn1c(C(=O)O)c(CCCOc3cc(C)c(Cl)c(C)c3)c3ccc(F)c-2c31]

To a stirred solution of (rac)-ethyl 1-(3-(4-chloro-3,5-dimethylphenoxy)propyl)-14-ethyl-15-fluoro-13-methyl-4,9,11,13-tetrahydrobenzo[3,4]pyrazolo[4′,3′:9,10][1,6]oxaazacycloundecino-[8,7,6-hi]indole-2-carboxylate (see intermediate 83, 53.0 mg, 82.0 μmol, 1.00 eq.) in ethanol (329 μL, 0.25 M) was added a 2.0 M solution of sodium hydroxide in water (102 μL, 206 μmol, 2.50 eq.). The resulting yellow solution was heated to 70° C. for 2 days and was then cooled to room temperature. The mixture was diluted with dimethylsulfoxide, acidified with trifluoroacetic acid and was then purified by reverse phase column chromatography (10-100% acetonitrile/water with 0.1% formic acid gradient) to give the title compound as as a white solid (45.3 mg).

LC-MS (Method 3): Rₜ=4.82 min, MS (ESIpos): m/z=616 [M+H]⁺.

¹H-NMR (300 MHz, DMSO-d) δ [ppm]: 13.24 (s, 1H), 7.70 (dd, 1H), 7.18 (m, 3H), 7.09 (m, 2H), 6.68 (s, 2H), 5.29 (d, 2H), 4.42 (d, 1H), 4.15 (m, 2H), 3.99 (d, 1H), 3.85 (m, 5H), 3.04 (m, 2H), 2.25 (m, 8H), 1.94 (m, 2H), 0.91 (t, 3H)

### Example 80

### (rac)-15-Fluoro-12,14-dimethyl-1-(3-(naphthalen-1-yloxy)propyl)-4,9,11,12-tetrahydrobenzo-[3,4]pyrazolo[4′,3′:9,10][1]oxa[6]azacycloundecino[8,7,6-hi]indole-2-carboxylic Acid

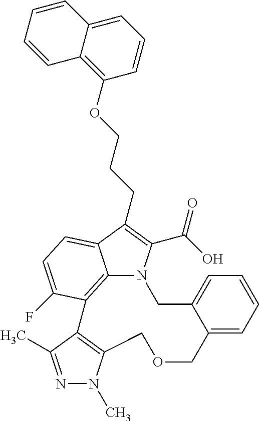 [SMILES: Cc1nn(C)c2c1-c1c(F)ccc3c(CCCOc4cccc5ccccc45)c(C(=O)O)n(c13)Cc1ccccc1COC2]

To a stirred solution of (rac)-ethyl 15-fluoro-12,14-dimethyl-1-(3-(naphthalen-1-yloxy)propyl)-4,9,11,12-tetrahydrobenzo[3,4]pyrazolo[4′,3′:9,10][1]oxa[6]azacycloundecino[8,7,6-hi]indole-2-carboxylate (see intermediate 88, 168 mg, 270 μmol, 1.00 eq.) in ethanol (1.08 mL, 0.25 M) was added a 2.0 M solution of sodium hydroxide in water (337 μL, 675 μmol, 2.50 eq.). The resulting yellow solution was heated to 70° C. for 22 hours, cooled to room temperature and was then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as a white solid (149.4 mg).

LC-MS (Method 3): Rₜ=4.51 min, MS (ESIpos): m/z=590 [M+H]⁺.

¹H-NMR (300 MHz, CHLOROFORM-d) δ [ppm]: 8.34 (m, 1H), 7.80 (m, 1H), 7.65 (dd, 1H), 7.47 (m, 2H), 7.41 (d, 1H), 7.32 (m, 2H), 7.13 (t, 1H), 7.01 (t, 1H), 6.85 (t, 1H), 6.72 (dd, 2H), 5.58 (d, 1H), 5.38 (d, 1H), 4.60 (d, 1H), 4.16 (m, 5H), 3.97 (s, 3H), 3.45 (m, 2H), 2.36 (m, 2H), 1.90 (s, 3H).

### Example 81

### (rac)-15-Fluoro-1-(3-((6-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylic Acid

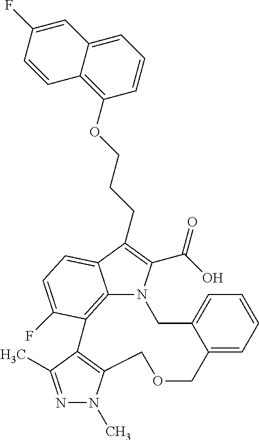 [SMILES: Cc1nn(C)c2c1-c1c(F)ccc3c(CCCOc4cccc5cc(F)ccc45)c(C(=O)O)n(c13)Cc1ccccc1COC2]

To a stirred solution of (rac)-ethyl 15-fluoro-1-(3-((6-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 89, 166 mg, 259 μmol, 1.00 eq.) in ethanol (1.03 mL, 0.25 M) was added a 2.0 M solution of sodium hydroxide in water (323 μL, 647 μmol, 2.50 eq.). The resulting yellow solution was heated to 70° C. for 22 hours, cooled to room temperature and was then acidified with 1.0 M aqueous hydrochloric acid. Celite was added to the resulting mixture, volatiles were removed under reduced pressure, and the residue subjected to reverse phase column chromatography (50-100% acetonitrile/water with 0.1% formic acid gradient) to give the title compound as a white solid (131 mg).

LC-MS (Method 3): Rₜ=4.59 min, MS (ESIpos): m/z=608 [M+H]⁺.

¹H-NMR (300 MHz, CHLOROFORM-d) δ [ppm]: 8.32 (dd, 1H), 7.63 (dd, 1H), 7.34 (m, 4H), 7.18 (m, 2H), 7.01 (t, 1H), 6.86 (t, 1H), 6.72 (d, 1H), 6.65 (m, 1H), 5.57 (d, 1H), 5.39 (d, 1H), 4.60 (d, 1H), 4.16 (m, 5H), 3.97 (s, 3H), 3.40 (m, 2H), 2.35 (m, 2H), 1.90 (s, 3H).

### Example 82

### (rac)-15-Fluoro-1-(3-((4-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylic Acid

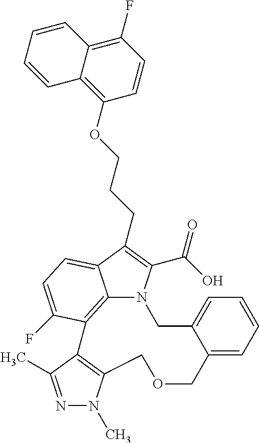 [SMILES: Cc1nn(C)c2c1-c1c(F)ccc3c(CCCOc4ccc(F)c5ccccc45)c(C(=O)O)n(c13)Cc1ccccc1COC2]

To a stirred solution of (rac)-ethyl 15-fluoro-1-(3-((4-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 90, 111 mg, 174 μmol, 1.00 eq.) in ethanol (696 μL, 0.25 M) was added a 2.0 M solution of sodium hydroxide in water (217 μL, 435 μmol, 2.50 eq.). The resulting yellow solution was heated to 70° C. for 22 hours, cooled to room temperature and then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as an off-white solid (96.0 mg).

LC-MS (Method 3): Rₜ=4.62 min, MS (ESIpos): m/z=608 [M+H]⁺.

¹H-NMR (300 MHz, CHLOROFORM-d) δ [ppm]: ¹H NMR (Chloroform-d) δ: 8.31 (dt, 1H), 8.03 (dd, 1H), 7.64 (dd, 1H), 7.53 (m, 2H), 7.28 (d, 1H), 7.12 (m, 1H), 6.98 (m, 2H), 6.86 (t, 1H), 6.71 (d, 1H), 6.56 (dd, 1H), 5.58 (d, 1H), 5.38 (d, 1H), 4.60 (d, 1H), 4.25 (d, 1H), 4.12 (m, 4H), 3.98 (s, 3H), 3.41 (m, 2H), 2.35 (m, 2H), 1.90 (s, 3H).

### Example 83

### (rac)-15-Fluoro-12,14-dimethyl-1-(3-((5,6,7,8-tetrahydronaphthalen-1-yl)oxy)propyl)-4,9,11,12-tetrahydrobenzo[3,4]pyrazolo[4′,3′:9,10][1]oxa[6]azacycloundecino[8,7,6-hi]indole-2-carboxylic Acid

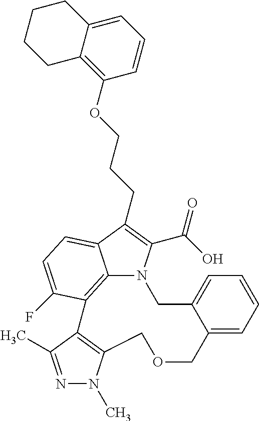 [SMILES: Cc1nn(C)c2c1-c1c(F)ccc3c(CCCOc4cccc5c4CCCC5)c(C(=O)O)n(c13)Cc1ccccc1COC2]

To a stirred solution of (rac)-ethyl 15-fluoro-12,14-dimethyl-1-(3-((5,6,7,8-tetrahydronaphthalen-1-yl)oxy)propyl)-4,9,11,12-tetrahydrobenzo[3,4]pyrazolo[4′,3′:9,10][1]-oxa[6]azacycloundecino[8,7,6-hi]indole-2-carboxylate (see intermediate 91, 104 mg, 270 μmol, 1.00 eq.) in ethanol (660 μL, 0.25 M) was added a 2.0 M solution of sodium hydroxide in water (337 μL, 675 μmol, 2.50 eq.). The resulting yellow solution was heated to 70° C. for 22 hours, cooled to room temperature and was then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as a white solid (81.4 mg).

LC-MS (Method 3): Rₜ=4.84 min, MS (ESIpos): m/z=594 [M+H]⁺.

¹H-NMR (300 MHz, CHLOROFORM-d) δ [ppm]: 7.64 (dd, 1H), 7.29 (d, 1H), 7.14 (t, 1H), 7.02 (t, 2H), 6.92 (t, 1H), 6.71 (dd, 2H), 6.56 (d, 1H), 5.58 (d, 1H), 5.39 (d, 1H), 4.61 (d, 1H), 4.22 (m, 2H), 4.10 (d, 1H), 3.99 (d, 5H), 3.32 (m, 2H), 2.73 (m, 4H), 2.21 (s, 2H), 1.90 (s, 3H), 1.77 (m, 4H).

### Example 84

### (rac)-1-(3-((2,3-Dihydro-1H-inden-4-yl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylic Acid

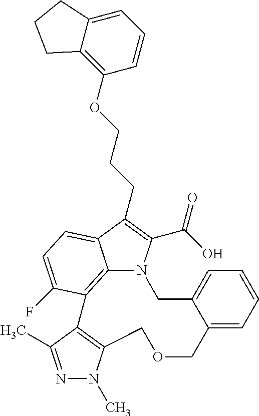 [SMILES: Cc1nn(C)c2c1-c1c(F)ccc3c(CCCOc4cccc5c4CCC5)c(C(=O)O)n(c13)Cc1ccccc1COC2]

To a stirred solution of (rac)-ethyl 1-(3-((2,3-dihydro-1H-inden-4-yl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 92, 94.4 mg, 155 μmol, 1.00 eq.) in ethanol (620 μL, 0.25 M) was added a 2.0 M solution of sodium hydroxide in water (193 μL, 387 μmol, 2.50 eq.). The resulting yellow solution was heated to 70° C. for 22 hours, cooled to room temperature and was then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as a white solid (83.0 mg).

LC-MS (Method 3): Rₜ=4.59 min, MS (ESIpos): m/z=580 [M+H]⁺.

¹H-NMR (300 MHz, CHLOROFORM-d) δ [ppm]: 7.64 (dd, 1H), 7.29 (s, 1H), 7.09 (m, 3H), 6.91 (t, 1H), 6.84 (d, 1H), 6.73 (d, 1H), 6.57 (d, 1H), 5.58 (d, 1H), 5.39 (d, 1H), 4.61 (d, 1H), 4.22 (m, 2H), 4.10 (d, 1H), 4.03 (m, 2H), 3.97 (s, 3H), 3.33 (m, 2H), 2.92 (t, 4H), 2.20 (m, 2H), 2.07 (m, 2H), 1.90 (s, 3H).

### Example 85

### (rac)-1-(3-(4-chloro-3,5-dimethylphenoxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylic Acid

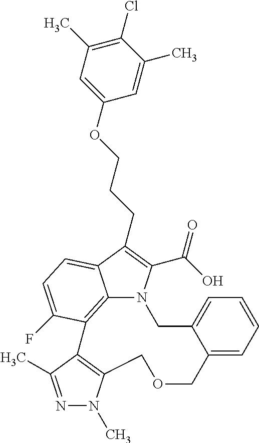 [SMILES: Cc1cc(OCCCc2c(C(=O)O)n3c4c(c(F)ccc24)-c2c(C)nn(C)c2COCc2ccccc2C3)cc(C)c1Cl]

To a stirred solution of (rac)-ethyl 1-(3-(4-chloro-3,5-dimethylphenoxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylate (see intermediate 93, 95.2 mg, 151 μmol, 1.00 eq.) in ethanol (604 μL, 0.25 M) was added a 2.0 M solution of sodium hydroxide in water (188 μL, 377 μmol, 2.50 eq.). The resulting yellow solution was heated to 70° C. for 22 hours, cooled to room temperature and was then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as a white solid (68.8 mg).

LC-MS (Method 3): Rₜ=4.77 min, MS (ESIpos): m/z=604 [M+H]⁺.

¹H-NMR (300 MHz, CHLOROFORM-d) δ [ppm]: 7.62 (dd, 1H), 7.12 (t, 1H), 7.00 (t, 1H), 6.91 (t, 1H), 6.72 (d, 1H), 6.58 (s, 2H), 5.56 (d, 1H), 5.39 (d, 1H), 4.61 (d, 1H), 4.17 (m, 3H), 3.95 (m, 5H), 3.29 (m, 2H), 2.31 (s, 6H), 2.19 (m, 2H), 1.90 (s, 3H).

### Example 86

### (rac)-1-(3-(4-Chloro-3,5-dimethylphenoxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylic Acid

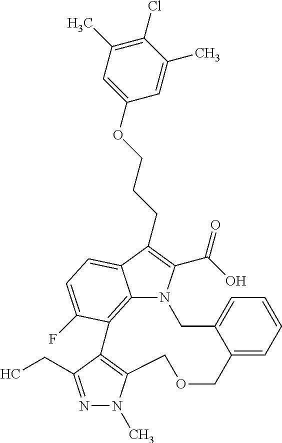 [SMILES: CCc1nn(C)c2c1-c1c(F)ccc3c(CCCOc4cc(C)c(Cl)c(C)c4)c(C(=O)O)n(c13)Cc1ccccc1COC2]

To a stirred solution of (rac)-ethyl 1-(3-(4-chloro-3,5-dimethylphenoxy)propyl)-14-ethyl-15-fluoro-12-methyl-4,9,11,12-tetrahydrobenzo[3,4]pyrazolo[4′,3′:9,10][1,6]oxaazacycloundecino-[8,7,6-hi]indole-2-carboxylate (see intermediate 98, 105 mg, 163 μmol, 1.00 eq.) in ethanol (0.65 mL, 0.25 M) was added a 2.0 M solution of sodium hydroxide in water (203 μL, 407 μmol, 2.50 eq.). The resulting yellow solution was heated to 70° C. for 20 hours, cooled to room temperature and was then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as a white solid (46.6 mg).

LC-MS (Method 3): Rₜ=4.94 min, MS (ESIpos): m/z=616 [M+H]⁺.

¹H-NMR (300 MHz, DMSO-d) δ [ppm]: 13.32 (s, 1H), 7.73 (dd, 1H), 7.20 (m, 2H), 7.06 (m, 2H), 6.82 (dd, 1H), 6.70 (s, 2H), 5.44 (d, 1H), 5.22 (d, 1H), 4.72 (d, 1H), 4.12 (m, 2H), 3.92 (m, 6H), 3.12 (tt, 2H), 2.25 (s, 6H), 2.08 (m, 4H), 0.87 (t, 3H)

### Example 87

### (rac)-1-(3-((2,3-Dihydro-1H-inden-4-yl)oxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylic Acid

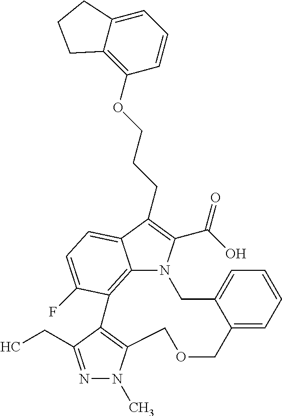 [SMILES: CCc1nn(C)c2c1-c1c(F)ccc3c(CCCOc4cccc5c4CCC5)c(C(=O)O)n(c13)Cc1ccccc1COC2]

To a stirred solution of (rac)-ethyl 1-(3-((2,3-dihydro-1H-inden-4-yl)oxy)propyl)-14-ethyl-15-fluoro-12-methyl-4,9,11,12-tetrahydrobenzo[3,4]pyrazolo[4′,3′:9,10][1,6]oxaazacycloundecino-[8,7,6-hi]indole-2-carboxylate (see intermediate 99, 100 mg, 161 μmol, 1.00 eq.) in ethanol (0.64 mL, 0.25 M) was added a 2.0 M solution of sodium hydroxide in water (201 μL, 402 μmol, 2.50 eq.). The resulting yellow solution was heated to 70° C. for 20 hours, cooled to room temperature and was then acidified with 1.0 M aqueous hydrochloric acid. Celite was added to the resulting mixture, volatiles were removed under reduced pressure, and the residue was subjected to reverse phase column chromatography (50-100% acetonitrile/water with 0.1% formic acid gradient) to give the title compound as a white solid (82.9 mg).

LC-MS (Method 3): Rₜ=4.75 min, MS (ESIpos): m/z=594 [M+H]⁺.

¹H-NMR (300 MHz, DMSO-d) δ [ppm]: 13.32 (s, 1H), 7.72 (dd, 1H), 7.12 (m, 5H), 6.82 (m, 2H), 6.59 (d, 1H), 5.43 (d, 1H), 5.22 (d, 1H), 4.72 (d, 1H), 4.10 (d, 2H), 3.93 (m, 6H), 3.14 (m, 2H), 2.81 (m, 4H), 2.07 (m, 6H), 0.88 (t, 3H).

### Example 88

### (rac)-14-Ethyl-15-fluoro-12-methyl-1-(3-((5,6,7,8-tetrahydronaphthalen-1-yl)oxy)propyl)-4,9,11,12-tetrahydrobenzo[3,4]pyrazolo[4′,3′:9,10][1,6]oxaazacycloundecino[8,7,6-hi]indole-2-carboxylic Acid

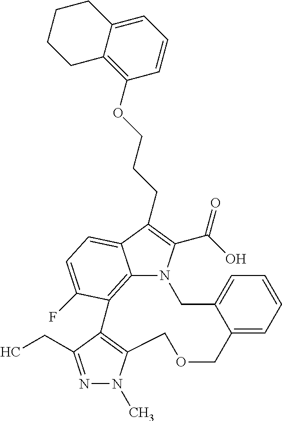 [SMILES: CCc1nn(C)c2c1-c1c(F)ccc3c(CCCOc4cccc5c4CCCC5)c(C(=O)O)n(c13)Cc1ccccc1COC2]

To a stirred solution of (rac)-ethyl 14-ethyl-15-fluoro-12-methyl-1-(3-((5,6,7,8-tetrahydronaphthalen-1-yl)oxy)propyl)-4,9,11,12-tetrahydrobenzo[3,4]pyrazolo[4′,3′:9,10][1,6]-oxaazacycloundecino[8,7,6-hi]indole-2-carboxylate (see intermediate 100, 108 mg, 170 μmol, 1.00 eq.) in ethanol (0.68 mL, 0.25 M) was added a 2.0 M solution of sodium hydroxide in water (212 μL, 424 μmol, 2.50 eq.). The resulting yellow solution was heated to 70° C. for 20 hours, cooled to room temperature and was then acidified with 1.0 M aqueous hydrochloric acid. Celite was added to the resulting mixture, volatiles were removed under reduced pressure, and the residue was subjected to reverse phase column chromatography (10-100% acetonitrile/water with 0.1% formic acid gradient) to give the title compound as a white solid (60.3 mg).

LC-MS (Method 3): Rₜ=5.01 min MS (ESIpos): m/z=608 [M+H]⁺.

¹H-NMR (300 MHz, DMSO-d) δ [ppm]: 13.33 (s, 1H), 7.72 (dd, 1H), 7.12 (m, 5H), 6.84 (dd, 1H), 6.60 (m, 2H), 5.43 (d, 1H), 5.23 (d, 1H), 4.72 (d, 1H), 4.10 (d, 2H), 3.93 (m, 6H), 3.15 (m, 2H), 2.67 (t, 2H), 2.59 (t, 2H), 2.10 (m, 4H), 1.68 (m, 4H), 0.88 (t, 3H).

### Example 89

### (rac)-14-Ethyl-15-fluoro-1-(3-((4-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylic Acid

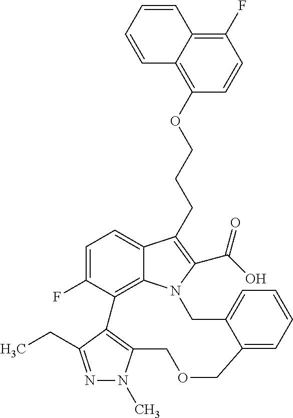 [SMILES: CCc1nn(C)c2c1-c1c(F)ccc3c(CCCOc4ccc(F)c5ccccc45)c(C(=O)O)n(c13)Cc1ccccc1COC2]

To a stirred solution of (rac)-ethyl 14-ethyl-15-fluoro-1-(3-((4-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylate (see intermediate 101, 115 mg, 177 μmol, 1.00 eq.) in ethanol (0.71 mL, 0.25 M) was added a 2.0 M solution of sodium hydroxide in water (221 μL, 442 μmol, 2.50 eq.). The resulting yellow solution was heated to 70° C. for 20 hours, cooled to room temperature and was then acidified with 1.0 M aqueous hydrochloric acid. Celite was added to the resulting mixture, volatiles were removed under reduced pressure, and the residue was was subjected to reverse phase column chromatography (10-100% acetonitrile/water with 0.1% formic acid gradient) to give the title compound as an off-white solid (46.1 mg).

LC-MS (Method 3): Rₜ=4.78 min, MS (ESIpos): m/z=622 [M+H]⁺.

¹H-NMR (300 MHz, DMSO-d) δ [ppm]: 13.39 (s, 1H), 8.25 (m, 1H), 7.99 (m, 1H), 7.75 (m, 1H), 7.63 (m, 2H), 7.20 (m, 3H), 7.10 (td, 1H), 6.97 (t, 1H), 6.81 (m, 2H), 5.44 (d, 1H), 5.23 (d, 1H), 4.71 (d, 1H), 4.11 (dd, 4H), 3.92 (d, 1H), 3.87 (s, 3H), 3.29 (m, 4H), 2.12 (m, 4H), 0.87 (t, 3H).

### Example 90

### (rac)-14-Ethyl-15-fluoro-1-(3-((6-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylic Acid

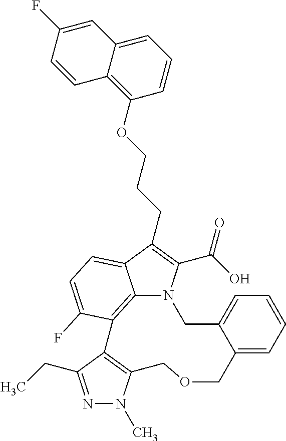 [SMILES: CCc1nn(C)c2c1-c1c(F)ccc3c(CCCOc4cccc5cc(F)ccc45)c(C(=O)O)n(c13)Cc1ccccc1COC2]

To a stirred solution of (rac)-ethyl 14-ethyl-15-fluoro-1-(3-((6-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylate (see intermediate 102, 181 mg, 279 μmol, 1.00 eq.) in ethanol (1.11 mL, 0.25 M) was added a 2.0 M solution of sodium hydroxide in water (348 μL, 696 μmol, 2.50 eq.). The resulting yellow solution was heated to 70° C. for 20 hours, cooled to room temperature and was then acidified with 1.0 M aqueous hydrochloric acid. Celite was added to the resulting mixture, volatiles were removed under reduced pressure, and the residue was subjected to reverse phase column chromatography (10-100% acetonitrile/water with 0.1% formic acid gradient) to give the title compound as an off-white solid (142 mg).

LC-MS (Method 3): Rₜ=4.74 min, MS (ESIpos): m/z=622 [M+H]⁺.

¹H-NMR (300 MHz, DMSO-d) δ [ppm]: 13.38 (s, 1H), 8.27 (m, 1H), 7.76 (dd, 1H), 7.65 (dd, 1H), 7.40 (m, 3H), 7.21 (m, 2H), 7.12 (m, 1H), 6.97 (t, 1H), 6.84 (m, 2H), 5.45 (d, 1H), 5.23 (d, 1H), 4.71 (d, 1H), 4.12 (m, 4H), 3.92 (d, 1H), 3.87 (s, 3H), 3.25 (dd, 2H), 2.13 (m, 4H), 0.87 (t, 3H).

### Example 91

### (rac)-14-Ethyl-15-fluoro-12-methyl-1-(3-(naphthalen-1-yloxy)propyl)-4,9,11,12-tetrahydrobenzo[3,4]pyrazolo[4′,3′:9,10][1,6]oxaazacycloundecino[8,7,6-hi]indole-2-carboxylic Acid

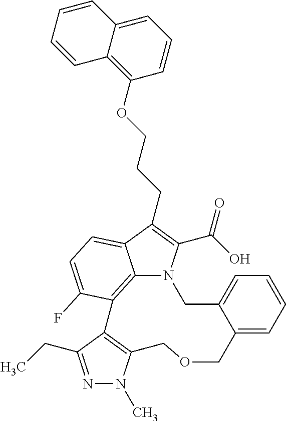 [SMILES: CCc1nn(C)c2c1-c1c(F)ccc3c(CCCOc4cccc5ccccc45)c(C(=O)O)n(c13)Cc1ccccc1COC2]

To a stirred solution of (rac)-ethyl 14-ethyl-15-fluoro-12-methyl-1-(3-(naphthalen-1-yloxy)propyl)-4,9,11,12-tetrahydrobenzo[3,4]pyrazolo[4′,3′:9,10][1,6]oxaazacycloundecino-[8,7,6-hi]indole-2-carboxylate (see intermediate 103, 169 mg, 268 μmol, 1.00 eq.) in ethanol (1.07 mL, 0.25 M) was added a 2.0 M solution of sodium hydroxide in water (334 μL, 669 μmol, 2.50 eq.). The resulting yellow solution was heated to 70° C. for 20 hours, cooled to room temperature and was then acidified with 1.0 M aqueous hydrochloric acid. Celite was added to the resulting mixture, volatiles were removed under reduced pressure, and the residue subjected to reverse phase column chromatography (10-100% acetonitrile/water with 0.1% formic acid gradient) to give the title compound as a white solid (127 mg).

LC-MS (Method 3): Rₜ=4.57 min, MS (ESIpos): m/z=604 [M+H]⁺.

¹H-NMR (300 MHz, DMSO-d) δ [ppm]: 13.40 (s, 1H), 8.23 (m, 1H), 7.82 (m, 2H), 7.51 (m, 2H), 7.41 (m, 2H), 7.17 (m, 3H), 6.97 (t, 1H), 6.85 (m, 2H), 5.45 (d, 1H), 5.23 (d, 1H), 4.71 (d, 1H), 4.13 (m, 4H), 3.92 (d, 1H), 3.87 (s, 3H), 3.25 (m, 2H), 2.14 (m, 4H), 0.88 (t, 3H).

### Example 92

### (rac)-14-Ethyl-15-fluoro-13-methyl-1-(3-((1,2,3,4-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylic Acid

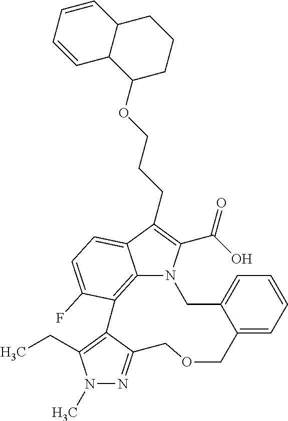 [SMILES: CCc1c2c(nn1C)COCc1ccccc1Cn1c(C(=O)O)c(CCCOC3CCCC4C=CC=CC43)c3ccc(F)c-2c31]

To a stirred solution of (rac)-ethyl 14-ethyl-15-fluoro-13-methyl-1-(3-((1,2,3,4-tetrahydronaphthalen-1-yl)oxy)propyl)-4,9,11,13-tetrahydrobenzo[3,4]pyrazolo[4′,3′:9,10][1]-oxa[6]azacycloundecino[8,7,6-hi]indole-2-carboxylate (see intermediate 81, 11.0 mg, 17.3 μmol, 1.00 eq.) in ethanol (69.2 μL, 0.25 M) was added a 2.0 M solution of sodium hydroxide in water (21.6 μL, 206 μmol, 2.50 eq.). The resulting yellow solution was heated to 70° C. for 2 days and then cooled to room temperature. The mixture was diluted with dimethyl sulfoxide, acidified with trifluoroacetic acid and was then purified by reverse phase column chromatography (10-100% acetonitrile/water with 0.1% formic acid gradient) to give the title compound as a white solid (6.95 mg).

LC-MS (Method 3): Rₜ=4.49 min, MS (ESIpos): m/z=608 [M+H]⁺.

¹H-NMR (300 MHz, CHLOROFORM-d) δ [ppm]: 7.57 (dd, 1H), 7.20 (m, 9H), 5.50 (dd, 1H), 5.33 (dd, 1H), 4.69 (dd, 1H), 4.48 (t, 1H), 4.32 (dd, 1H), 4.20 (m, 3H), 3.93 (s, 3H), 3.36 (m, 1H), 3.14 (m, 3H), 2.67 (m, 2H), 2.41 (m, 2H), 1.94 (m, 3H), 1.55 (m, 3H), 1.25 (m, 1H), 1.04 (t, 3H), 0.84 (m, 1H).

### Example 93

### (rac)-3-Chloro-17-{3-[(6-fluoronaphthalen-1-yl)oxy]propyl}-4,5-dimethyl-5,7,9,14-tetrahydropyrazino[2′,3′:3,4]pyrazolo[4′,3′:9,10][1,6]oxazacycloundecino[8,7,6-hi]indole-16-carboxylic Acid

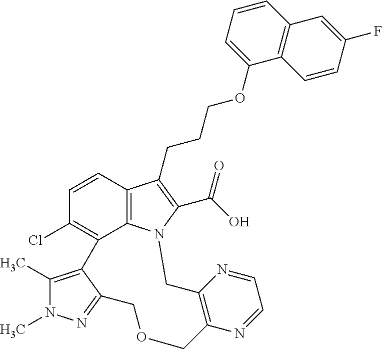 [SMILES: Cc1c2c(nn1C)COCc1nccnc1Cn1c(C(=O)O)c(CCCOc3cccc4cc(F)ccc34)c3ccc(Cl)c-2c31]

(rac)-Ethyl-3-chloro-17-{3-[(6-fluoronaphthalen-1-yl)oxy]propyl}-4,5-dimethyl-5,7,9,14-tetrahydropyrazino[2′,3′:3,4]pyrazolo[4′,3′:9,10][1,6]oxazacycloundecino[8,7,6-hi]indole-16-carboxylate (see intermediate 51, 89.0 mg, 50% purity, 68.0 μmol) was dissolved in a mixture of 1 mL of THF and 100 μL of ethanol, and aqueous lithium hydroxide solution (140 μL, 1.0 M, 140 μmol) was added. The mixture was stirred at 70° C. for 20 hours in a sealed tube. Aqueous lithium hydroxide solution (140 μL, 1.0 M, 140 μmol) was added, and stirring was continued at 70° C. for 3 days in a sealed tube. The reaction mixture was diluted with water and acidified using aqueous, saturated citric acid solution until a pH value of 3-4 was reached. The mixture was extracted with ethyl acetate and the combined organic layers were concentrated under reduced pressure. The crude material was purified by flash chromatography using a silica gel (gradient dichloromethane/ethanol). The product was further purified by preparative HPLC (Method P3) to obtain the title compound (7.9 mg, 19% yield).

LC-MS (Method 1): Rₜ=1.46 min, MS (ESIpos): m/z=626 [M+H]⁺

¹H-NMR (400 MHz, DMSO-d6) δ [ppm]: 1.232 (2.08), 1.846 (16.00), 2.073 (1.46), 2.133 (1.46), 2.151 (2.13), 2.167 (1.56), 2.327 (1.54), 2.669 (1.56), 3.220 (1.32), 3.235 (2.35), 3.251 (2.62), 3.269 (1.94), 3.848 (12.68), 4.087 (0.92), 4.095 (0.92), 4.111 (1.43), 4.126 (0.70), 4.142 (0.73), 4.157 (1.48), 4.173 (0.97), 4.181 (1.00), 4.207 (1.54), 4.232 (1.73), 4.367 (1.73), 4.399 (2.19), 4.486 (2.02), 4.511 (1.78), 4.560 (1.51), 4.591 (1.16), 5.050 (1.51), 5.088 (1.83), 5.526 (1.54), 5.563 (1.40), 6.799 (1.54), 6.806 (1.62), 6.813 (1.54), 6.820 (1.67), 7.169 (3.24), 7.190 (3.51), 7.347 (0.94), 7.354 (1.05), 7.369 (1.70), 7.376 (1.83), 7.392 (1.05), 7.399 (1.19), 7.425 (3.10), 7.432 (3.64), 7.438 (6.69), 7.607 (3.26), 7.628 (3.21), 7.635 (2.13), 7.642 (2.05), 7.661 (1.86), 7.668 (1.89), 8.169 (0.73), 8.220 (1.56), 8.234 (1.70), 8.243 (1.67), 8.257 (1.56), 8.317 (3.40), 8.323 (4.26), 8.355 (4.45), 8.362 (3.53).

### Example 94

### (rac)-3-chloro-4,5-dimethyl-16-{3-[(naphthalen-1-yl)oxy]propyl}-5,7-dihydro-9H,13H-[1,2]oxazolo[3′,4′:3,4]pyrazolo[4′,3′:9,10][1,6]oxazacycloundecino[8,7,6-hi]indole-15-carboxylic Acid or (rac)-3-chloro-4,5-dimethyl-16-[3-(1-naphthyloxy)propyl]-5,7-dihydro-9H,13H-[1,2]oxazolo[4′,3′:3,4]pyrazolo[4′,3′:9,10][1,6]oxazacycloundecino[8,7,6-hi]indole-15-carboxylic Acid (it is not Determined which Regio-Isomer was Synthesized)

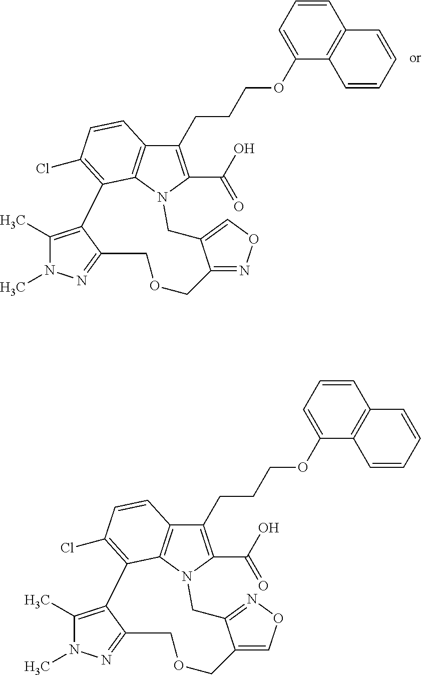 [SMILES: Cc1c2c(nn1C)COCc1nocc1Cn1c(C(=O)O)c(CCCOc3cccc4ccccc34)c3ccc(Cl)c-2c31 ; Cc1c2c(nn1C)COCc1conc1Cn1c(C(=O)O)c(CCCOc3cccc4ccccc34)c3ccc(Cl)c-2c31]

(rac)-Ethyl-3-chloro-4,5-dimethyl-16-{3-[(naphthalen-1-yl)oxy]propyl}-5,7-dihydro-9H,13H-[1,2]oxazolo[3′,4′:3,4]pyrazolo[4′,3′:9,10][1,6]oxazacycloundecino[8,7,6-hi]indole-15-carboxylate (see intermediate 114, 35.0 mg) was dissolved in a mixture of 1.3 mL tetrahydrofuran and 1.3 mL of ethanol and an aqueous solution of lithium hydroxide (1.1 mL, 1.0 M, 1.1 mmol) was added. The reaction was heated to 60° C. for 3 days. The mixture was concentrated, the residue diluted with water, acidified with 2N aqueous hydrochloric acid and extracted with dichloromethane thrice. The combined organic extracts were dried over magnesium sulfate and evaporated. The crude material was purified by preparative HPLC, eluting a gradient of 70% to 80% acetonitrile in a 0.1% aqueous solution of formic acid. Appropriate fractions were combined and lyophilised to give 3.3 mg of the title compound.

¹H NMR (CDCl3, 400 MHz): 2.00, 2.09 (2×s, 3H), 2.22-2.36 (m 2H), 3.30-3.68 (m, 3H), 3.87, 3.92 (2×s, 3H), 4.14, 4.20 (2×t, 2H), 4.25-4.79 (m, 4H), 4.91, 5.55 (2×d, 1H), 6.74 (dd, 1H), 7.19 (dd, 1H), 7.31-7.37 (m, 1H), 7.38-7.44 (m, 1H), 7.45-7.52 (m, 2H), 7.58-7.64 (m, 1H), 7.77-7.83 (m, 1H), 8.25 (s, 1H), 8.31-8.37 (m, 1H).

UPLC1 (CSH C18 long acid 2-95%): Rt=3.09 min, MS (ESIpos): [M+H]+ 597.

### Experimental Section—Biological Assays

Examples were tested in selected biological assays one or more times. When tested more than once, data are reported as either average values, median values or as geometric mean values, wherein
the average value, also referred to as the arithmetic mean value, represents the sum of the values obtained divided by the number of times tested,
the median value represents the middle number of the group of values when ranked in ascending or descending order. If the number of values in the data set is odd, the median is the middle value. If the number of values in the data set is even, the median is the arithmetic mean of the two middle values, and
the geometric mean value represents the nth root of the product of n numbers.

Examples were synthesized one or more times. When synthesized more than once, data from biological assays represent average values or median values calculated utilizing data sets obtained from testing of one or more synthetic batch.

An empty field in any of the following tables means that the respective compound has not been tested in that Assay.

The in vitro activity of the compounds of the present invention can be demonstrated in the following assays.

Assay 1

Protein-Protein Interaction Assay: MCL-1/Noxa BH3 Peptide (MCL-1 Assay)

The dose-dependent inhibition by the compounds described in this invention of the interaction between MCL-1 and the BH3 domain of Noxa (both human) was determined using a steady state binding competition assay with time-resolved fluorescence energy transfer (TR-FRET) readout. For that purpose MCL-1 (amino acids 173-321, N-terminal fused to Maltose Binding Protein (MBP), SEQ ID 1) and a synthetic Noxa BH3-derived peptide of sequence Biotin-PEG2-PEG2-PAELEVE-Nva-ATQLRRFGDKLNFRQKLL-amide (SEQ ID 2) served as protein receptor and tracer ligand respectively. The MBP-MCL-1 was purchased from Beryllium (Bedford, Mass., USA). The expression and purification of this protein construct has been described elsewhere (DOI:10.1371/journal.pone.0125010). The Noxa BH3-derived peptide can be obtained from e.g. Biosyntan (Berlin, Germany).

In the assay 11 different concentrations of each compound (0.1 nM, 0.33 nM, 1.1 nM, 3.8 nM, 13 nM, 44 nM, 0.15 μM, 0.51 μM, 1.7 μM, 5.9 μM and 20 μM) were typically measured as duplicates in the same microtiter plate. For that, 100-fold concentrated DMSO solutions were prepared by serial dilutions (1:3.4) of a 2 mM stock solution in a clear, 384-well microtiter plate (Greiner Bio-One, Frickenhausen, Germany). From there, 50 nl were transferred in a dark test plate (Greiner Bio-One, Frickenhausen, Germany). The assay was initiated by addition of 2 μl of a 2,5-fold concentrated MBP-MCL-1 solution (usually for a 1 nM end concentration in 5 μl reaction volume) in aqueous assay buffer [50 mM Tris/HCl pH 7, 100 mM sodium chloride (NaCl), 50 mM potassium fluoride (KF), 0.005% Tween-20, 2 mM DTT, 0.1% bovine gamma globulin (BGG)] to the compounds in the assay plate. This was followed by a 10-minute incubation step at 22° C. for pre-equilibration of the putative complex between MBP-MCL-1 and the compounds. After that, 3 μl of a 1.67-fold concentrated solution (in assay buffer) consisting of Noxa BH3-derived peptide (1 nM end concentration) and TR-FRET detection reagents [1.67 nM anti-MBP-Eu cryptate and 1.67 nM streptavidin-XL665 (both from Cisbio Bioassays, Codolet, France)], were added.

The mixture was incubated in the dark for one hour at 22° C. and then overnight at 4° C. The formation of MCL-1/Noxa complexes was determined by measuring the resonance energy transfer of the anti-MBP-Eu-cryptate antibody to the streptavidin-XL665 present in the reaction. For that purpose, the fluorescence emission at 620 nm and 665 nm after excitation at 330-350 nm was measured in a TR-FRET measuring instrument, for instance a Rubystar or a Pherastar (both from BMG Lab Technologies, Offenburg, Germany) or a Viewlux (Perkin-Elmer). The ratio of the emission at 665 nm and at 622 nm was used as indicator of the amount of MCL-1/NOXA complexes present.

The resulting data (ratio) were normalized, taking 0% inhibition as the mean value of control measurements (usually 32 data points) where all reagents were included. In this case 50 nl DMSO were used instead of compounds. A 100% inhibition corresponded to the mean value of control measurements (usually 32 data points) where all reagents except MCL-1 were included. IC₅₀ values were determined by regression analysis based on a 4 parameter equation (minimum, maximum, IC₅₀, Hill; Y=Max+(Min−Max)/(1+(X/IC₅₀)\hat{ }Hill) using the Screener Software (Genedata).

SEQ ID 1:
GKIEEGKLVIWINGDKGYNGLAEVGKKFEKDTGIKVTVEHPDKLEEKFP
QVAATGDGPDIIFWAHDRFGGYAQSGLLAEITPDKAFQDKLYPFTWDAV
RYNGKLIAYPIAVEALSLIYNKDLLPNPPKTWEEIPALDKELKAKGKSA
LMFNLQEPYFTWPLIAADGGYAFKYENGKYDIKDVGVDNAGAKAGLTFL
VDLIKNKHMNADTDYSIAEAAFNKGETAMTINGPWAWSNIDTSKVNYGV
TVLPTFKGQPSKPFVGVLSAGINAASPNKELAKEFLENYLLTDEGLEAV
NKDKPLGAVALKSYEEELAKDPRIAATMENAQKGEIMPNIPQMSAFWYA
VRTAVINAASGRQTVDEALKDAQTGSSELYRQSLEIISRYLREQATGAA
DTAPMGASGATSRKALETLRRVGDGVQRNHETAFQGMLRKLDIKNEDDV
KSLSRVMIHVFSDGVTNWGRIVTLISFGAFVAKHLKTINQESCIEPLAE
SITDVLVRTKRDWLVKQRGWDGFVEFFHV
SEQ ID 2:
Biotin-PEG2-PEG2-PAELEVE-Nva-ATQLRRFGDKLNFRQKLL-
amide

Assay 2

Protein-Protein Interaction Assay: BCL-XL/Bad BH3 Peptide (BCL-XL Assay)

The dose-dependent inhibition by the compounds described in this invention of the interaction between BCL-XL and the BH3 domain of Bad (both human) was determined using a steady state binding competition assay with time-resolved fluorescence energy transfer (TR-FRET) readout. For that purpose BCL-XL (amino acids 1-212, C-terminal fused to a hexahistidine (6×His) tag (SEQ ID 3) and a synthetic Bad BH3-derived peptide of sequence Biotin-PEG2-PEG2-NLWAAQRYGRELRR-Nle-SDEFVDSFKK-amide (SEQ ID 4) served as protein receptor and tracer ligand respectively. The recombinant BCL-XL protein (expressed in E. coli) was purchased from BPS Bioscience (San Diego, Calif., USA). The Bad BH3-derived peptide can be obtained from e.g. Biosyntan (Berlin, Germany).

In the assay 11 different concentrations of each compound (0.1 nM, 0.33 nM, 1.1 nM, 3.8 nM, 13 nM, 44 nM, 0.15 μM, 0.51 μM, 1.7 μM, 5.9 μM and 20 μM) were typically measured as duplicates in the same microtiter plate. For that, 100-fold concentrated DMSO solutions were prepared by serial dilutions (1:3.4) of a 2 mM stock solution in a clear, 384-well microtiter plate (Greiner Bio-One, Frickenhausen, Germany). From there, 50 nl were transferred in a dark test plate (Greiner Bio-One, Frickenhausen, Germany). The assay was initiated by addition of 2 μl of a 2,5-fold concentrated His-BCL-XL solution (usually for a 1 nM end concentration in 5 μl reaction volume) in aqueous assay buffer [50 mM Tris/HCl pH 7, 100 mM sodium chloride (NaCl), 50 mM potassium fluoride (KF), 0.005% Tween-20, 2 mM DTT, 0.1% bovine gamma globulin (BGG)] to the compounds in the assay plate. This was followed by a 10-minute incubation step at 22° C. for pre-equilibration of the putative complex between His-BCL-XL and the compounds. After that, 3 μl of a 1.67-fold concentrated solution (in assay buffer) consisting of Bad BH3-derived peptide (1 nM end concentration) and TR-FRET detection reagents [1.67 nM anti-His-Eu cryptate and 1.67 nM streptavidin-XL665 (both from Cisbio Bioassays, Codolet, France)], were added.

The mixture was incubated in the dark for one hour at 22° C. and then overnight at 4° C. The formation of BCL-XL/Bad complexes was determined by measuring the resonance energy transfer of the anti-His- Eu-cryptate antibody to the streptavidin-XL665 present in the reaction. For that purpose, the fluorescence emission at 620 nm and 665 nm after excitation at 330-350 nm was measured in a TR-FRET measuring instrument, for instance a Rubystar or a Pherastar (both from BMG Lab Technologies, Offenburg, Germany) or a Viewlux (Perkin-Elmer). The ratio of the emission at 665 nm and at 622 nm was used as indicator of the amount of BCL-XL/Bad complexes present.

The resulting data (ratio) were normalized, taking 0% inhibition as the mean value of control measurements (usually 32 data points) where all reagents were included. In this case 50 nl DMSO were used instead of compounds. A 100% inhibition corresponded to the mean value of control measurements (usually 32 data points) where all reagents except BCL-XL were included. IC₅₀ values were determined by regression analysis based on a 4 parameter equation (minimum, maximum, IC₅₀, Hill; Y=Max+(Min−Max)/(1+(X/IC₅₀)\hat{ }Hill) using the Screener Software (Genedata).

MSQSNRELVV DFLSYKLSQK GYSWSQFSDV EENRTEAPEG
TESEMETPSA INGNPSWHLA DSPAVNGATG HSSSLDAREV
IPMAAVKQAL REAGDEFELR YRRAFSDLTS QLHITPGTAY
QSFEQVVNEL FRDGVNWGRI VAFFSFGGAL CVESVDKEMQ
VLVSRIAAWM ATYLNDHLEP WIQENGGWDT FVELYGNNAA
AESRKGQERF NR
SEQ ID 3
Biotin-PEG2-PEG2-NLWAAQRYGRELRR-Nle-SDEFVDSFKK-
amide
SEQ ID 4

Assay 3

Protein-Protein Interaction Assay: BCL-2/Bad BH3 Peptide (BCL-2 Assay)

The dose-dependent inhibition by the compounds described in this invention of the interaction between BCL-2 and the BH3 domain of Bad (both human) was determined using a steady state binding competition assay with time-resolved fluorescence energy transfer (TR-FRET) readout. For that purpose BCL-2 (amino acids 1-211, C-terminal fused to a hexahistidine (6×His) tag (SEQ ID 5) and a synthetic Bad BH3-derived peptide of sequence Biotin-PEG2-PEG2-NLWAAQRYGRELRR-Nle-SDEFVDSFKK-amide (SEQ ID 4) served as protein receptor and tracer ligand respectively. The recombinant BCL-2 protein (expressed in E. coli) was purchased from BPS Bioscience (San Diego, Calif., USA). The Bad BH3-derived peptide can be obtained from e.g. Biosyntan (Berlin, Germany).

In the assay 11 different concentrations of each compound (0.1 nM, 0.33 nM, 1.1 nM, 3.8 nM, 13 nM, 44 nM, 0.15 μM, 0.51 μM, 1.7 μM, 5.9 μM and 20 μM) were typically measured as duplicates in the same microtiter plate. For that, 100-fold concentrated DMSO solutions were prepared by serial dilutions (1:3.4) of a 2 mM stock solution in a clear, 384-well microtiter plate (Greiner Bio-One, Frickenhausen, Germany). From there, 50 nl were transferred in a dark test plate (Greiner Bio-One, Frickenhausen, Germany). The assay was initiated by addition of 2 μl of a 2,5-fold concentrated His-BCL-2 solution (usually for a 1 nM end concentration in 5 μl reaction volume) in aqueous assay buffer [50 mM Tris/HCl pH 7, 100 mM sodium chloride (NaCl), 50 mM potassium fluoride (KF), 0.005% Tween-20, 2 mM DTT, 0.1% bovine gamma globulin (BGG)] to the compounds in the assay plate. This was followed by a 10-minute incubation step at 22° C. for pre-equilibration of the putative complex between His-BCL-2 and the compounds. After that, 3 μl of a 1.67-fold concentrated solution (in assay buffer) consisting of Bad BH3-derived peptide (1 nM end concentration) and TR-FRET detection reagents [1.67 nM anti-His-Eu cryptate and 1.67 nM streptavidin-XL665 (both from Cisbio Bioassays, Codolet, France)], were added.

The mixture was incubated in the dark for one hour at 22° C. and then overnight at 4° C. The formation of BCL-2/Bad complexes was determined by measuring the resonance energy transfer of the anti-His-Eu-cryptate antibody to the streptavidin-XL665 present in the reaction. For that purpose, the fluorescence emission at 620 nm and 665 nm after excitation at 330-350 nm was measured in a TR-FRET measuring instrument, for instance a Rubystar or a Pherastar (both from BMG Lab Technologies, Offenburg, Germany) or a Viewlux (Perkin-Elmer). The ratio of the emission at 665 nm and at 622 nm was used as indicator of the amount of BCL-2/Bad complexes present.

The resulting data (ratio) were normalized, taking 0% inhibition as the mean value of control measurements (usually 32 data points) where all reagents were included. In this case 50 nl DMSO were used instead of compounds. A 100% inhibition corresponded to the mean value of control measurements (usually 32 data points) where all reagents except BCL-2 were included. IC₅₀ values were determined by regression analysis based on a 4 parameter equation (minimum, maximum, IC₅₀, Hill; Y=Max+(Min−Max)/(1+(X/IC₅₀)\hat{ }Hill) using the Screener Software (Genedata).

SEQ ID 5:
MAHAGRTGYD NREIVMKYIH YKLSQRGYEW DAGDVGAAPP
GAAPAPGIFS SQPGHTPHPA ASRDPVARTS PLQTPAAPGA
AAGPALSPVP PVVHLTLRQA GDDFSRRYRR DFAEMSSQLH
LTPFTARGRF ATVVEELFRD GVNWGRIVAF FEFGGVMCVE
SVNREMSPLV DNIALWMTEY LNRHLHTWIQ DNGGWDAFVE
LYGPSMRPLF D

TABLE 2
IC₅₀ values of selected examples in biochemical
MCL-1 assay (Assay 1) and biochemical BCL-2
assay (Assay 3), BCL-XL assay (Assay 2)

| | MCL-1 | BCL-2 | BCL-XL |
|---|---|---|---|
| | Assay [M] | Assay [M] | Assay [M] |
| Example | (median) | (median) | (median) |
| 1 | 1.4E−8 | | |
| 2 | 2.3E−9 | >2.00E−05 | >2.00E−05 |
| 3 | 2.7E−8 | | |
| 4 | 5.3E−9 | | |
| 5 | 2.9E−9 | | |
| 6 | 1.1E−8 | | |
| 7 | 1.7E−9 | | |
| 8 | 1.5E−9 | >2.00E−05 | >2.00E−05 |
| 9 | 3.2E−8 | | |
| 10 | 2.8E−9 | >2.00E−05 | >2.00E−05 |
| 11 | 5.4E−9 | | |
| 12 | 2.9E−9 | >2.00E−05 | >2.00E−05 |
| 13 | 1.7E−7 | | |
| 14 | 5.2E−9 | | |
| 15 | 6.9E−9 | >2.00E−05 | >2.00E−05 |
| 16 | 1.1E−7 | | |
| 17 | 3.4E−8 | | |
| 18 | 2.9E−8 | | |
| 19 | 9.4E−7 | | |
| 20 | 1.8E−7 | | |
| 21 | 3.5E−8 | | |
| 22 | 6.5E−7 | | |
| 23 | 3.9E−8 | | |
| 24 | 5.3E−8 | | |
| 25 | 2.6E−7 | | |
| 26 | 3.8E−8 | | |
| 27 | 3.1E−8 | | |
| 28 | 2.8E−7 | | |
| 29 | 3.0E−8 | | |
| 30 | 3.7E−8 | | |
| 31 | 1.3E−8 | | |
| 32 | 1.8E−7 | | |
| 33 | 1.7E−9 | | |
| 34 | 6.6E−10 | >2.00E−05 | >2.00E−05 |
| 35 | 5.4E−9 | | |
| 36 | 3.2E−9 | | |
| 37 | 1.3E−9 | >2.00E−05 | >2.00E−05 |
| 38 | 7.1E−9 | | |
| 39 | 1.6E−9 | | |
| 40 | 2.8E−9 | >2.00E−05 | 1.94E−5 |
| 41 | 5.8E−9 | | |
| 42 | 1.1E−9 | | |
| 43 | 5.1E−10 | >2.00E−05 | >2.00E−05 |
| 44 | 5.2E−9 | | |
| 45 | 2.5E−9 | | |
| 46 | 1.6E−9 | >2.00E−05 | >2.00E−05 |
| 47 | 9.5E−9 | | |
| 48 | 5.9E−10 | | |
| 49 | 4.7E−10 | 2.18E−5 | >2.00E−05 |
| 50 | 1.4E−9 | | |
| 51 | 1.5E−9 | | |
| 52 | 9.6E−10 | >2.00E−05 | >2.00E−05 |
| 53 | 8.3E−9 | | |
| 54 | 8.3E−10 | | |
| 55 | 1.1E−9 | 1.80E−5 | >2.00E−05 |
| 56 | 3.2E−9 | | |
| 57 | 2.7E−9 | | |
| 58 | 2.6E−9 | 1.06E−5 | 1.73E−5 |
| 59 | 1.0E−8 | | |
| 60 | 2.9E−9 | 1.98E−5 | >2.00E−05 |
| 61 | 2.1E−9 | >2.00E−05 | >2.00E−05 |
| 62 | 2.6E−9 | >2.00E−05 | >2.00E−05 |
| 63 | 4.5E−9 | 1.81E−5 | >2.00E−05 |
| 64 | 1.5E−9 | | |
| 65 | 1.3E−9 | 1.69E−5 | >2.00E−05 |
| 66 | 6.2E−9 | | |
| 67 | 1.7E−9 | | |
| 68 | 3.2E−9 | >2.00E−05 | >2.00E−05 |
| 69 | 2.1E−8 | | |
| 70 | 2.1E−9 | | |
| 71 | 1.2E−9 | 1.28E−5 | >2.00E−05 |
| 72 | 5.2E−9 | | |
| 73 | 7.8E−10 | | |
| 74 | 9.2E−10 | 1.98E−5 | >2.00E−05 |
| 75 | 3.0E−9 | | |
| 76 | 3.1E−9 | 1.58E−5 | >2.00E−05 |
| 77 | 2.8E−9 | 1.26E−5 | >2.00E−05 |
| 78 | 1.9E−9 | >2.00E−05 | >2.00E−05 |
| 79 | 2.6E−9 | 1.56E−5 | 1.96E−5 |
| 80 | 7.6E−9 | >2.00E−05 | >2.00E−05 |
| 81 | 4.6E−9 | 1.80E−5 | >2.00E−05 |
| 82 | 1.2E−8 | | |
| 83 | 1.0E−8 | | |
| 84 | 6.2E−9 | >2.00E−05 | >2.00E−05 |
| 85 | 1.2E−8 | | |
| 86 | 1.1E−8 | | |
| 87 | 4.5E−9 | 1.37E−5 | >2.00E−05 |
| 88 | 7.4E−9 | 1.37E−5 | >2.00E−05 |
| 89 | 1.2E−8 | | |
| 90 | 3.2E−9 | 1.78E−5 | >2.00E−05 |
| 91 | 8.6E−9 | 2.05E−5 | >2.00E−05 |
| 92 | 1.8E−8 | | |
| 93 | 1.5E−9 | >2.00E−05 | >2.00E−05 |
| 94 | 2.8.E−9 | | |

Cellular Assays

Assay 4

Induction of Caspase-3/7 Activity Upon Treatment of Cells with Selected Compounds

The BH3-domain of MCL-1 sequesters pro-apoptotic proteins, thereby inhibiting apoptosis. In contrast, MCL-1 inhibitors are expected to antagonize this effect leading to an increase in apoptosis, which can be determined by measuring the activity of caspase-3/7.

The activity of caspase-3/7 was determined in DLBCL (Diffuse large B-cell lymphoma) cell lines (SUDHL5 and SUDHL10) upon treatment with different compounds, using the Caspase-Glo® 3/7 reagent from Promega (G8092).

The different cell lines were plated in culture medium (RPMI 1640 [GIBCO #22400-089] supplemented with 10% Fetal Bovine Serum) at a density of 3,300 cells in 30 μl/well in a sterile, solid black, flat bottom, polystyrene, TC-treated 384-well microplate (Corning #3571) using Multidrop Combi Reagent Dispenser. As a control, medium without cells was also added to the plate. Cells were incubated in a humidified incubator at 37° C. overnight.

On the next day, the cells were treated with compounds (stock solution, 10 mM in DMSO) using the HP D300 Digital Dispenser in a concentration range of 3.3×10e-5 M (33 μM) to 5×10e-9 M (5 nM) in a single-dot curve with at least 16 dilutions and a DMSO concentration of 0.33%. Rim wells were excluded. The cells were incubated for 3 hours in a humidified incubator at 37° C. After this incubation, 30 μl of Caspase-Glo® 3/7 reagent (Promega G8092) was added to each well using the Multidrop Combi Reagent Dispenser, followed by 1 hour incubation at 37° C. Finally, luminescence was read at 0.1 ms, with a gain of 3000 using the PHERAstar FS microplate reader (BMG Labtech).

For the evaluation of the results, the background measured with “medium-only” was subtracted from all other values. Then, the values were normalized to DMSO-only treated cells (every value was divided by the mean of the DMSO control). The Bella DRC Master Sheet was used to calculate EC₅₀s, with fixed C0=1 and CI=plateau/max induction for the reference compound.

TABLE 3
EC₅₀ values of selected examples in cellular
caspase induction assay (Assay 4)

| | Caspase | Caspase |
|---|---|---|
| | SUDHL5 [M] | SUDHL10 [M] |
| Example | (median) | (median) |
| 1 | 1.0E−5 | >3.3E−5 |
| 2 | 2.0E−6 | 7.7E−6 |
| 3 | >1.2E−5 | >3.3E−5 |
| 4 | 1.5E−5 | >3.2E−5 |
| 5 | 5.4E−6 | 1.4E−5 |
| 6 | >2.2E−5 | >3.3E−5 |
| 7 | 3.0E−6 | 1.6E−5 |
| 8 | 2.5E−6 | 9.9E−6 |
| 9 | >3.3E−5 | >3.3E−5 |
| 10 | 4.3E−6 | 1.8E−5 |
| 11 | 5.6E−6 | 1.6E−5 |
| 12 | 2.4E−6 | 4.9E−6 |
| 13 | 1.4E−5 | >3.3E−5 |
| 14 | 2.7E−6 | 7.6E−6 |
| 15 | 2.6E−6 | 1.2E−5 |
| 16 | 2.4E−5 | >3.3E−5 |
| 17 | 3.0E−6 | 8.5E−6 |
| 18 | 9.2E−7 | 5.2E−6 |
| 19 | 1.7E−5 | >3.3E−5 |
| 20 | >3.3E−5 | >3.3E−5 |
| 21 | 2.0E−5 | >3.3E−5 |
| 22 | 1.4E−5 | >3.3E−5 |
| 23 | 4.2E−6 | 1.2E−5 |
| 24 | >3.3E−5 | >3.3E−5 |
| 25 | >3.3E−5 | >3.3E−5 |
| 26 | 1.8E−5 | >3.3E−5 |
| 27 | >3.3E−5 | >3.3E−5 |
| 28 | >3.3E−5 | >3.3E−5 |
| 29 | 1.0E−5 | 2.3E−5 |
| 30 | 8.5E−6 | >3.3E−5 |
| 31 | 5.2E−6 | 9.7E−6 |
| 32 | >3.3E−5 | >3.3E−5 |
| 33 | 1.9E−6 | 4.9E−6 |
| 34 | 4.9E−7 | 1.0E−6 |
| 35 | 4.2E−6 | 1.3E−5 |
| 36 | 2.4E−6 | 5.5E−6 |
| 37 | 8.4E−7 | 2.5E−6 |
| 38 | 1.7E−6 | 1.1E−5 |
| 39 | 3.7E−6 | 5.7E−6 |
| 40 | 2.5E−6 | 1.4E−5 |
| 41 | 6.1E−6 | 1.6E−5 |
| 42 | 1.5E−6 | 3.0E−6 |
| 43 | 1.2E−6 | 3.7E−6 |
| 44 | 7.8E−6 | 1.5E−5 |
| 45 | 2.2E−6 | 7.9E−6 |
| 46 | 1.8E−6 | 7.3E−6 |
| 47 | 4.7E−6 | 1.6E−5 |
| 48 | 1.1E−6 | 3.1E−6 |
| 49 | 7.7E−7 | 2.1E−6 |
| 50 | 7.3E−7 | 3.2E−6 |
| 51 | 8.7E−7 | 1.8E−6 |
| 52 | 4.9E−7 | 7.1E−7 |
| 53 | 1.8E−6 | 5.9E−6 |
| 54 | 8.4E−7 | 1.8E−6 |
| 55 | 6.6E−7 | 8.7E−7 |
| 56 | 1.3E−6 | 5.4E−6 |
| 57 | 1.5E−6 | 4.3E−6 |
| 58 | 8.0E−7 | 2.1E−6 |
| 59 | 1.9E−6 | 7.9E−6 |
| 60 | 3.0E−6 | 1.2E−5 |
| 61 | 2.5E−6 | 5.8E−6 |
| 62 | 3.2E−6 | 1.0E−5 |
| 63 | 9.3E−6 | 2.3E−5 |
| 64 | 1.7E−6 | 6.0E−6 |
| 65 | 8.1E−7 | 2.1E−6 |
| 66 | 3.3E−6 | 1.2E−5 |
| 67 | 2.2E−6 | 5.9E−6 |
| 68 | 1.2E−6 | 1.1E−5 |
| 69 | 1.3E−5 | >3.3E−5 |
| 70 | 1.6E−6 | 8.6E−6 |
| 71 | 1.6E−6 | 4.0E−6 |
| 72 | 1.2E−5 | >2.8E−5 |
| 73 | 9.9E−7 | 3.1E−6 |
| 74 | 8.2E−7 | 1.2E−6 |
| 75 | 4.4E−6 | 1.5E−5 |
| 76 | 3.3E−6 | 1.3E−5 |
| 77 | 2.4E−6 | 8.4E−6 |
| 78 | 2.1E−6 | 5.2E−6 |
| 79 | 2.9E−6 | 1.3E−5 |
| 80 | 5.2E−6 | >2.5E−5 |
| 81 | 2.1E−6 | 5.2E−6 |
| 82 | 2.2E−5 | >3.3E−5 |
| 83 | 4.6E−6 | 1.2E−5 |
| 84 | 9.8E−6 | 2.7E−5 |
| 85 | 3.7E−6 | 2.0E−5 |
| 86 | 2.7E−6 | 6.9E−6 |
| 87 | 2.3E−6 | 9.4E−6 |
| 88 | 6.2E−6 | 1.7E−5 |
| 89 | 2.9E−6 | 8.3E−6 |
| 90 | 1.4E−6 | 6.6E−6 |
| 91 | 2.8E−6 | 1.2E−5 |
| 92 | 1.5E−5 | >3.3E−5 |
| 93 | 2.5E−6 | 1.0E−5 |
| 94 | 8.3E−7 | 2.3E−6 |

Assay 5

PIxEL: Protein-Protein Interaction in Permeabilized Cells by ELISA

Most MCL1 protein molecules are localized at the mitochondria outer membrane and sequester pro-apoptotic proteins through binding of their BCL2 homology domain 3 (BH3 domain). MEB buffer (150 mM mannitol, 10 mM HEPES pH 7.5, 50 mM KCl, 20 μM EDTA, 20 μM EGTA, 5 mM potassium succinate, 0.1% protease-free BSA (SIGMA) with low dose digitonin (0.002%) permeabilizes plasma membrane while leaves live mitochondria, where MCL1 maintains its native localization and conformation. Unlike biophysical assays (e.g. TR-FRET) that use truncated recombinant MCL1 protein, this assay uses full length endogenous MCL1 protein at mitochondria outer membrane. It measures the interaction between MCL1 protein and biotinylated BIM BH3 peptide. Compounds can compete with BIM BH3 peptide to bind to MCL1 protein. This serum free assay measures the affinity between MCL1 protein and compound in permeabilized cells, therefore it is not affected by serum binding and cell permeability, and can measure the intrinsic compound affinity.

On day 1, RKO colon cancer cell line cells were plated at 0.8 million cells/ml, 100 μl/well in 96-well flat bottom TC plates (Corning). MCL1 antibody (Santa Cruz sc-12756) were diluted at 200 fold (final concentration 1 μg/ml) in carbonate buffer (Thermo Fisher Scientific, pH 9.6), and 50 μl of diluted antibody was added to each well of high bind ELISA plates (SARSTEDT). Each plate was tapped to make sure liquid covering entire bottom of wells and incubate at 37° C. overnight.

On the second day, MCL1 antibody was washed from ELISA plate. 250 μl Odyssey® Blocking Buffer (PBS) (Li-Cor) was added to each well, incubated at room temperature for at least 1 hour, then washed once with 250 μl 1×PBST. Plates with RKO cells were gently washed once with 100 μl/well PBS, once with 100 μl/well MEB buffer without digitonin, then 100 μl of MEB buffer with 0.002% digitonin was gently added to each well. Compounds were added with HP Tecan compound dispenser in 3-fold dilution series, highest dose 30 μM, 10-dose per compound in quadruplicates. Biotin-BIM peptide (synthesized by 21st Century) was added with HP Tecan compound dispenser at 0.2 μM immediately after the addition of compounds. Plates were rocked for 1 h at room temperature. Then MEB buffer was aspirated and 50 μl of CHAPS buffer (50 mM Tris-Cl, pH 7.4, 150 mM NaCl, 1% CHAPS, 1 mM EDTA, 1 mM EGTA, complete protease inhibitors (Roche), PhosSTOP (Roche)) was added to each well. Plates were rocked for 1 hour at 4° C., then 45 μl cell lysate from each well were transferred to ELISA plates coated with MCL1 antibody. Plates were incubated overnight in the cold room with rocking.

On the third day, ELISA plates were washed once with 250 μl 1×PBST. Streptavidin-poly-HRP (Thermo Fisher Scientific) was diluted to 20 ng/ml in Odyssey blocking buffer plus 0.05% Triton-100, and 100 μl was added to each well of the ELISA plate. Plates were incubated at RT for 1 hour with rocking, then washed with 100 μL 1×PBST for 3 times. Each SuperSignal ELISA Femto Maximum Sensitivity substrate was added to a 50-ml tube and mixed, then 100 μl of mixed substrate was added to each well. Plates were shaken for 1 minute then luminescence was measured by Envision plate reader (HP). Signal of each well were normalized by no-compound control and no-cell control. IC₅₀ was calculated using Graphic Pad PRISM software.

Table 4 shows the results of the protein-protein interaction in permeabilized cells by ELISA assay (Assay 5).

TABLE 4
IC₅₀ values of selected examples in protein-protein interaction
in permeabilized cells by ELISA assay (Assay 5)

| Example | PIxEL [M] (median) |
|---|---|
| 1 | |
| 2 | |
| 3 | |
| 4 | |
| 5 | |
| 6 | |
| 7 | |
| 8 | 1.82E−05 |
| 9 | |
| 10 | |
| 11 | |
| 12 | |
| 13 | |
| 14 | |
| 15 | |
| 16 | |
| 17 | |
| 18 | |
| 19 | |
| 20 | |
| 21 | |
| 22 | |
| 23 | |
| 24 | |
| 25 | |
| 26 | |
| 27 | |
| 28 | |
| 29 | |
| 30 | |
| 31 | |
| 32 | |
| 33 | |
| 34 | 1.11E−06 |
| 35 | 1.85E−05 |
| 36 | |
| 37 | 1.61E−06 |
| 38 | |
| 39 | |
| 40 | |
| 41 | |
| 42 | |
| 43 | 2.97E−06 |
| 44 | |
| 45 | |
| 46 | 4.49E−06 |
| 47 | 1.80E−05 |
| 48 | |
| 49 | 7.88E−07 |
| 50 | 1.69E−06 |
| 51 | |
| 52 | 3.68E−07 |
| 53 | |
| 54 | |
| 55 | 3.11E−07 |
| 56 | 2.35E−06 |
| 57 | |
| 58 | 8.34E−07 |
| 59 | |
| 60 | |
| 61 | |
| 62 | |
| 63 | |
| 64 | |
| 65 | 1.04E−06 |
| 66 | |
| 67 | |
| 68 | 2.16E−06 |
| 69 | |
| 70 | |
| 71 | |
| 72 | |
| 73 | |
| 74 | |
| 75 | |
| 76 | |
| 77 | |
| 78 | |
| 79 | |
| 80 | |
| 81 | |
| 82 | |
| 83 | |
| 84 | |
| 85 | |
| 86 | |
| 87 | |
| 88 | |
| 89 | |
| 90 | |
| 91 | |
| 92 | |
| 93 | |
| 94 | 3.68E−06 |

Assay 6

Induction of Cytotoxicity Upon Treatment of Cells with Selected Compounds

In principle, compounds that induce apoptosis will concomitantly induce cell cytotoxicity. Therefore, cytotoxicity assays were run in parallel in SUDHL5 and SUDHL10 cells.

The different cell lines were plated in culture medium (RPMI 1640 [GIBCO #22400-089] supplemented with 10% Fetal Bovine Serum) at a density of 3,300 cells in 30 μl/well in a sterile, solid black, flat bottom, polystyrene, TC-treated 384-well microplate (Corning #3571) using Multidrop Combi Reagent Dispenser. As a control, medium without cells was also added to the plate. Cells were incubated in a humidified incubator at 37° C. overnight. On the next day, the cells were treated with compounds (stock solution, 10 mM in DMSO) using the HP D300 Digital Dispenser in a concentration range of 3.3×10e-5 M (33 μM) to 5×10e-9 M (5 nM) in a single-dot curve with at least 16 dilutions and a DMSO concentration of 0.33%. Rim wells were excluded. The cells were incubated for 5 hours in a humidified incubator at 37° C. After this incubation, 30 μl of CellTiter-Glo® Luminescent Cell Viability reagent (Promega, G7573) was added to each well using the Multidrop Combi Reagent Dispenser, followed by 15 minutes incubation on a shaker at room temperature. Finally, luminescence was read at 0.1 ms, with a gain of 3000 using the PHERAstar FS microplate reader (BMG Labtech).

For the evaluation of the results, each value was normalized to DMSO-only treated cells (every value was divided by the mean of the DMSO control). The Bella DRC Master Sheet was used to calculate IC₅₀s, with fixed CI=0 and C0=1.

Assay 7

Assessment of the Anti-Proliferative Effect of Compounds in Different Cell Lines

The impact of compounds on the proliferation of different cell lines was assessed using the CellTiter-Glo® Luminescent Cell Viability reagent from Promega (G7573). The cell lines used for the proliferation assays are examples of tumor indications and listed in the table below.

TABLE 5
cell lines, sources and indications

| Cell line | Source | Indication |
|---|---|---|
| SUDHL5 | DSMZ | B-cell lymphoma (GC-DLBCL) |
| SUDHL10 | DSMZ | B-cell lymphoma (GC-DLBCL) |
| MV-4-11 | ATCC | Acute monocytic leukemia |
| HMC-1-8 | JCRB | Triple-negative Breast Cancer |
| AMO-1 | DSMZ | Multiple Myeloma |
| A2780 | ECACC | Ovarian carcinoma |

The different cell lines were plated in culture medium (RPMI 1640 [Biochrom; #FG 1215] supplemented with 10% Fetal Calf Serum [Biochrom; #S 0415]) at a density of 3,300 cells (for suspension cells) or 800 cells (for adherent cells) in 30 μl/well in a sterile, solid black, flat bottom, polystyrene, TC-treated 384-well microplate (Corning #3571) using Multidrop Combi Reagent Dispenser. In parallel, cells were plated in a reference (day 0) plate for time zero determination. Cells were incubated in a humidified incubator at 37° C. overnight.

On the next day, cells were treated with compounds (stock solution, 10 mM in DMSO) using the HP D300 Digital Dispenser in a concentration range of 3.3×10e-5 M (33 μM) to 5×10e-9 M (5 nM) in a single-dot curve with at least 16 dilutions and a DMSO concentration of 0.33%. Rim wells were excluded. The cells were incubated for 72 hours in a humidified incubator at 37° C. The day 0 plate was measured by adding 30 μL/well of CTG solution (CellTiter-Glo® Luminescent Cell Viability reagent, Promega G7573) to time zero wells in the reference plate followed by a 10 minutes incubation and luminescence reading at 0.1 ms. using the PHERAstar FS microplate reader (BMG Labtech).

After 72 hours incubation, the treated plates were measured in the same way as the day 0 plate mentioned above. The Bella DRC Master Sheet was used to calculate IC₅₀s, with CI=day 0 values and C0=DMSO control values.

Table 6 shows the results of the SUDHL5 and SUDHL10 cytotoxicity and antiproliferation assays.

TABLE 6
IC₅₀ values of selected examples in cellular cytotoxicity induction
assay (Assay 5) and antiproliferation assay (Assay 6)

| | Cytotox | Cytotox | Antiproli | Antiproli |
|---|---|---|---|---|
| | SUDHL5 | SUDHL10 | SUDHL5 | SUDHL10 |
| | [M] | [M] | [M] | [M] |
| Example | (median) | (median) | (median) | (median) |
| 1 | 1.1E−5 | >1.6E−5 | 9.0E−6 | |
| 2 | 2.3E−6 | 7.3E−6 | 2.9E−6 | |
| 3 | 8.3E−6 | >3.3E−5 | 7.7E−6 | |
| 4 | 1.2E−5 | >2.3E−5 | 1.4E−5 | |
| 5 | 4.6E−6 | 1.3E−5 | 5.2E−6 | |
| 6 | >1.9E−5 | >3.3E−5 | 1.2E−5 | |
| 7 | 3.6E−6 | 1.5E−5 | 4.6E−6 | |
| 8 | 3.0E−6 | 7.2E−6 | 3.2E−6 | |
| 9 | >3.3E−5 | >3.3E−5 | 2.0E−5 | |
| 10 | 5.6E−6 | 1.4E−5 | 5.1E−6 | |
| 11 | 5.6E−6 | 1.5E−5 | 7.2E−6 | |
| 12 | 1.9E−6 | 5.0E−6 | | |
| 13 | 1.7E−5 | 2.5E−5 | | |
| 14 | 3.0E−6 | 7.0E−6 | 3.4E−6 | |
| 15 | 3.3E−6 | 1.1E−5 | 2.8E−6 | |
| 16 | 2.4E−5 | 3.0E−5 | | |
| 17 | 3.1E−6 | 1.4E−5 | 3.9E−6 | |
| 18 | 1.4E−6 | 3.8E−6 | | |
| 19 | 2.3E−5 | >3.3E−5 | | |
| 20 | >3.3E−5 | >3.3E−5 | 1.2E−5 | |
| 21 | 1.3E−5 | >3.3E−5 | 1.1E−5 | |
| 22 | 1.7E−5 | >3.3E−5 | | |
| 23 | 4.4E−6 | 1.1E−5 | | |
| 24 | >3.3E−5 | >3.3E−5 | >3.3E−5 | |
| 25 | >3.3E−5 | >3.3E−5 | | |
| 26 | 1.6E−5 | >3.3E−5 | | |
| 27 | >3.3E−5 | >3.3E−5 | 2.7E−5 | |
| 28 | >3.3E−5 | >3.3E−5 | | |
| 29 | 1.4E−5 | 2.3E−5 | | |
| 30 | 8.6E−6 | 2.9E−5 | 4.6E−6 | |
| 31 | 5.3E−6 | >7.8E−5 | 4.8E−6 | |
| 32 | >3.3E−5 | >3.3E−5 | 4.5E−6 | |
| 33 | 1.6E−6 | 3.5E−6 | 1.9E−6 | |
| 34 | 6.8E−7 | 1.4E−6 | 4.8E−7 | 1.2E−6 |
| 35 | 7.0E−6 | 1.4E−5 | 7.1E−6 | |
| 36 | 3.8E−6 | 4.9E−6 | 3.6E−6 | |
| 37 | 8.6E−7 | 2.7E−6 | | |
| 38 | 2.3E−6 | 6.8E−6 | | |
| 39 | 3.3E−6 | 6.7E−6 | 2.4E−6 | |
| 40 | 3.3E−6 | 8.3E−6 | 2.2E−6 | |
| 41 | 6.0E−6 | 1.4E−5 | 5.2E−6 | |
| 42 | 1.7E−6 | 3.5E−6 | 1.5E−6 | |
| 43 | 1.4E−6 | 2.9E−6 | 1.2E−6 | 1.5E−6 |
| 44 | 7.9E−6 | 1.5E−5 | 5.7E−6 | |
| 45 | 2.7E−6 | 8.2E−6 | 2.5E−6 | |
| 46 | 1.8E−6 | 7.0E−6 | 2.5E−6 | |
| 47 | 4.8E−6 | 1.9E−5 | 4.6E−6 | |
| 48 | 1.4E−6 | 2.6E−6 | 1.6E−6 | |
| 49 | 8.8E−7 | 1.6E−6 | 1.0E−6 | 1.2E−6 |
| 50 | 9.3E−7 | 2.8E−6 | 1.5E−6 | 1.2E−6 |
| 51 | 1.1E−6 | 1.9E−6 | 7.1E−7 | |
| 52 | 5.2E−7 | 8.8E−7 | 5.7E−7 | |
| 53 | 2.3E−6 | 6.5E−6 | 2.8E−6 | |
| 54 | 9.3E−7 | 2.6E−6 | 1.4E−6 | |
| 55 | 6.4E−7 | 1.3E−6 | 1.0E−6 | |
| 56 | 1.6E−6 | 3.7E−6 | 2.1E−6 | |
| 57 | 1.7E−6 | 4.6E−6 | 2.4E−6 | |
| 58 | 8.1E−7 | 2.6E−6 | 1.2E−6 | |
| 59 | 2.2E−6 | 8.2E−6 | 2.6E−6 | |
| 60 | 3.7E−6 | 7.6E−6 | 5.1E−6 | |
| 61 | 2.3E−6 | 5.0E−6 | 2.1E−6 | |
| 62 | 2.9E−6 | 5.2E−6 | 3.1E−6 | |
| 63 | 8.3E−6 | 1.5E−5 | 6.2E−6 | |
| 64 | 2.2E−6 | 3.2E−6 | 1.5E−6 | |
| 65 | 1.0E−6 | 2.1E−6 | 1.1E−6 | |
| 66 | 4.1E−6 | 8.6E−6 | 3.6E−6 | |
| 67 | 1.8E−6 | 4.9E−6 | 2.4E−6 | |
| 68 | 1.9E−6 | 4.6E−6 | 1.4E−6 | |
| 69 | 1.4E−5 | >3.3E−5 | 1.2E−5 | |
| 70 | 1.7E−6 | 6.1E−6 | 2.2E−6 | |
| 71 | 1.9E−6 | 3.6E−6 | 2.5E−6 | |
| 72 | 8.5E−6 | 2.6E−5 | 1.2E−5 | |
| 73 | 1.1E−6 | 3.1E−6 | 1.5E−6 | 1.5E−6 |
| 74 | 1.1E−6 | 1.4E−6 | 1.3E−6 | |
| 75 | 5.2E−6 | 1.0E−5 | 5.8E−6 | |
| 76 | 3.4E−6 | 9.4E−6 | 3.7E−6 | |
| 77 | 3.0E−6 | 6.2E−6 | 5.8E−6 | |
| 78 | 2.1E−6 | 3.7E−6 | 3.3E−6 | 1.9E−6 |
| 79 | 2.1E−6 | 1.0E−5 | 9.2E−6 | |
| 80 | 4.3E−6 | >1.1E−5 | 7.7E−6 | |
| 81 | 2.5E−6 | 4.0E−6 | 3.3E−6 | 3.0E−6 |
| 82 | 2.4E−5 | >3.3E−5 | 1.0E−5 | |
| 83 | 3.4E−6 | 5.5E−6 | 4.9E−6 | |
| 84 | 1.1E−5 | 2.2E−5 | 7.7E−6 | |
| 85 | 3.3E−6 | 6.5E−6 | 6.9E−6 | |
| 86 | 2.9E−6 | 5.2E−6 | 4.3E−6 | |
| 87 | 3.2E−6 | 6.0E−6 | 2.1E−6 | |
| 88 | 7.4E−6 | 1.5E−5 | 6.0E−6 | |
| 89 | 2.6E−6 | 7.5E−6 | 4.0E−6 | |
| 90 | 1.7E−6 | 3.5E−6 | 2.0E−6 | 2.1E−6 |
| 91 | 3.2E−6 | 7.5E−6 | 3.6E−6 | |
| 92 | 8.1E−6 | >3.3E−5 | >3.3E−5 | |
| 93 | 2.8E−6 | 6.7E−6 | 2.5E−6 | |
| 94 | 1.1E−6 | 1.5E−6 | 2.1E−6 | |

Table 7 shows the results of the MV4-11, AMO-1, HMC-1-8, A2780 proliferation assays.

TABLE 7
IC₅₀ values of selected examples in
antiproliferation assay (Assay 6)

| | Antiproli | Antiproli | Antiproli | Antiproli |
|---|---|---|---|---|
| Example | MV-4-11 [M] | AMO-1 [M] | HMC-1-8 [M] | A2780 [M] |
| 01 | 1.97E−5 | | | >3.30E−5 |
| | 2.36E−5 | | | 3.26E−5 |
| 02 | 1.77E−5 | | | 2.09E−5 |
| | 1.09E−5 | | | 1.49E−5 |
| | 1.32E−5 | | | 2.19E−5 |
| 03 | 1.15E−5 | | | 1.82E−5 |
| | 1.29E−5 | | | 1.94E−5 |
| | 9.57E−6 | | | 2.57E−5 |
| 04 | 2.09E−5 | | | 3.11E−5 |
| | 1.01E−5 | | | >3.30E−5 |
| 05 | 5.48E−6 | 8.78E−6 | | 6.65E−6 |
| 06 | 5.38E−6 | 1.99E−5 | | 8.66E−6 |
| 07 | 5.15E−6 | | | 2.96E−5 |
| | 5.50E−6 | | | >3.30E−5 |
| 08 | 7.10E−6 | 8.95E−6 | | >3.30E−5 |
| | 6.71E−6 | | | |
| 09 | 3.17E−5 | | | >3.30E−5 |
| 10 | 2.14E−5 | | | >3.30E−5 |
| | 8.09E−6 | | | 2.06E−5 |
| 11 | 7.59E−6 | | | 1.18E−5 |
| | 7.42E−6 | | | 1.22E−5 |
| 12 | | | | |
| 13 | | | | |
| 14 | 6.41E−6 | | | 1.75E−5 |
| 15 | 6.82E−6 | 6.25E−6 | | >3.30E−5 |
| | 2.99E−6 | 5.63E−6 | | |
| 16 | 1.15E−5 | 2.03E−5 | | |
| 17 | 5.93E−6 | | | 4.91E−6 |
| | 2.39E−6 | | | 5.77E−6 |
| 18 | | | | |
| 19 | | | | |
| 20 | 1.08E−5 | | | 1.37E−5 |
| 21 | 8.33E−6 | | | 1.19E−5 |
| 22 | | | | |
| 23 | | | | |
| 24 | >3.30E−5 | | | >3.30E−5 |
| 25 | | | | |
| 26 | | | | |
| 27 | 2.48E−5 | | | 3.14E−5 |
| 28 | | | | |
| 29 | | | | |
| 30 | 7.85E−6 | | | 1.71E−5 |
| 31 | 5.49E−6 | 5.93E−6 | | 6.72E−6 |
| 32 | 8.42E−6 | 1.35E−5 | | 1.08E−5 |
| 33 | 3.98E−6 | | | 9.04E−6 |
| | 4.97E−6 | | | 6.67E−6 |
| 34 | 1.73E−6 | 2.95E−6 | | 5.03E−6 |
| | 1.51E−6 | | | 4.68E−6 |
| | 1.95E−6 | | | |
| 35 | 1.19E−5 | | | 9.21E−6 |
| 36 | 1.38E−6 | | | 2.31E−5 |
| 37 | | | | |
| 38 | | | | |
| 39 | 1.56E−5 | | | >3.30E−5 |
| 40 | 1.50E−5 | 7.53E−6 | | 2.19E−5 |
| | 4.11E−6 | 9.07E−6 | | |
| 41 | 1.59E−5 | 8.40E−6 | | >3.30E−5 |
| | 8.20E−6 | 1.12E−5 | | |
| 42 | 3.34E−6 | 1.14E−5 | | 1.95E−5 |
| | 6.69E−6 | | | |
| 43 | 3.23E−6 | 5.16E−6 | | 2.34E−5 |
| | 2.83E−6 | 7.47E−6 | | |
| | 4.42E−6 | | | |
| 44 | 1.54E−5 | | | 2.37E−5 |
| 45 | 1.19E−5 | 9.56E−6 | | |
| 46 | 5.27E−6 | 6.15E−6 | 1.19E−5 | |
| 47 | 1.17E−5 | 1.15E−5 | 1.61E−5 | |
| 48 | 4.13E−6 | | | 1.05E−5 |
| | 4.81E−6 | | | 2.82E−5 |
| 49 | 3.09E−6 | 1.92E−6 | | 7.62E−6 |
| | 3.41E−6 | | | 5.26E−6 |
| | 3.78E−6 | | | |
| 50 | 2.21E−6 | 2.82E−6 | | 1.17E−5 |
| | 3.07E−6 | | | 8.65E−6 |
| | 2.53E−6 | | | |
| 51 | 1.96E−6 | 4.26E−6 | | |
| | 2.58E−6 | 2.64E−6 | | |
| 52 | 1.07E−6 | 2.27E−6 | | 2.41E−6 |
| | 2.66E−6 | | | |
| 53 | 4.91E−6 | 9.44E−6 | | 8.15E−6 |
| | 8.20E−6 | | | |
| 54 | 3.83E−6 | 1.77E−6 | | 6.59E−6 |
| 55 | 9.64E−7 | 1.19E−6 | 2.39E−6 | |
| 56 | 2.77E−6 | 4.20E−6 | 1.09E−5 | |
| 57 | 2.19E−6 | 2.65E−6 | | 7.02E−6 |
| 58 | 1.36E−6 | 1.61E−6 | 3.87E−6 | |
| 59 | 4.50E−6 | 7.14E−6 | | |
| 60 | 5.75E−6 | | | 2.30E−5 |
| 61 | 3.57E−6 | | | 2.28E−5 |
| 62 | 6.36E−6 | | | 1.39E−5 |
| 63 | >3.30E−5 | | | 1.97E−5 |
| 64 | 5.30E−6 | 7.46E−6 | | 1.71E−5 |
| | 1.07E−5 | | | |
| 65 | 2.94E−6 | 2.00E−6 | | 1.01E−5 |
| | 1.14E−6 | 1.75E−6 | | |
| 66 | 1.00E−5 | 7.13E−6 | | >3.30E−5 |
| | 4.01E−6 | 7.07E−6 | | |
| 67 | 3.08E−6 | | | 2.05E−5 |
| 68 | 7.51E−6 | 4.40E−6 | | 2.90E−5 |
| | 2.11E−6 | 4.89E−6 | | |
| 69 | 1.51E−5 | 2.60E−5 | | |
| 70 | 2.41E−6 | | | 2.28E−5 |
| | 2.27E−6 | | | 2.25E−5 |
| 71 | 4.81E−6 | 3.48E−6 | | 9.41E−6 |
| 72 | 9.57E−6 | 1.89E−5 | | 1.50E−5 |
| 73 | 3.82E−6 | 4.04E−6 | | 1.12E−5 |
| | 2.45E−6 | | | 1.42E−5 |
| | 2.83E−6 | | | |
| 74 | 3.65E−6 | 1.78E−6 | | |
| 75 | 1.64E−5 | 1.25E−5 | | |
| 76 | 9.05E−6 | | | 1.66E−5 |
| | 1.01E−5 | | | 2.50E−5 |
| 77 | 2.38E−6 | 7.22E−6 | | 1.02E−5 |
| | 8.66E−6 | | | |
| 78 | 8.37E−7 | 6.61E−6 | | 5.04E−6 |
| | 5.61E−6 | 6.82E−6 | | |
| | 7.38E−6 | | | |
| 79 | 2.33E−6 | | | 1.70E−5 |
| 80 | 5.10E−6 | 2.57E−5 | | 1.78E−5 |
| | 2.53E−5 | | | |
| 81 | 7.69E−6 | 6.52E−6 | | |
| | 7.82E−6 | 7.08E−6 | | |
| | 6.41E−6 | 4.13E−6 | | |
| 82 | 1.45E−5 | | | 2.85E−5 |
| 83 | 1.18E−5 | 1.61E−5 | | |
| 84 | 1.25E−5 | | | 3.10E−5 |
| 85 | 1.70E−5 | 2.37E−5 | | |
| 86 | 1.42E−5 | 1.69E−5 | | |
| 87 | 1.24E−5 | 1.29E−5 | | |
| | 6.98E−6 | 6.64E−6 | | |
| 88 | 9.93E−6 | | | 1.51E−5 |
| 89 | 8.44E−6 | 1.28E−5 | | |
| 90 | 4.98E−6 | 5.58E−6 | | |
| | 8.53E−6 | 1.03E−5 | | |
| | 6.99E−6 | 6.91E−6 | | |
| 91 | 1.24E−5 | 1.87E−5 | | |
| | 1.17E−5 | 1.53E−5 | | |
| 92 | 6.15E−6 | | | >3.30E−5 |
| 93 | 1.51E−5 | 7.19E−6 | 3.2E−5 | >3.30E−5 |
| 94 | 3.39E−6 | 6.44E−6 | 7.4E−6 | |
| | | 4.6E−6 | | |
| | | 1.0E−5 | | |

Assay 8

Protein-Compound Interaction Assay (SPR Assay)

The ability of the compounds described in this invention to bind to MCL-1 may be determined using surface plasmon resonance (SPR). This allows for the quantification of binding in terms of the equilibrium dissociation constant (KD [M]), as well as association and dissociation rate constants (kon [1/M 1/s] and koff [1/s], respectively). The measurements may be performed using Biacore® T200 or Biacore® S200 instruments (GE Healthcare).

For SPR measurements, recombinant MCL-1 (amino acids 173-321, N-terminal fused to Maltose Binding Protein (MBP) (SEQ ID 1) purchased from Beryllium (Bedford, Mass., USA)) was immobilized using standard amine coupling (Johnsson B et al, Anal Biochem. 1991 Nov. 1; 198(2):268-77). Briefly, carboxymethylated dextran biosensor chips (Series S Sensor Chip CM5, GE Healthcare) were activated with 1-ethyl-3-(3-dimethylaminopropyl)-carbodiimide hydrochloride (EDC) and N-hydroxysuccinimide (NHS) according to the supplier's instructions. MBP-MCL-1 was diluted in 1×PBS-P+ (GE Healthcare) and injected on the activated chip surface. Subsequently, a solution of 1 M ethanolamine-HCl (GE Healthcare) was injected to block unreacted groups, resulting in approximately 400-2500 response units (RU) of immobilized protein. A reference surface was generated by treatment with NHS-EDC and ethanolamine-HCl. Compounds were dissolved in 100% dimethylsulfoxide (DMSO) to a concentration of 10 mM and subsequently diluted in running buffer (1×PBS-P+ (GE Healthcare) [generated from PBS-P+ Buffer 10× (GE Healthcare): 0.2 M phosphate buffer with 27 mM KCl, 1.37 M NaCl and 0.5% Surfactant P20 (Tween 20)], 1% v/v DMSO). For SPR binding-measurements, serial dilutions of compound (eight dilution steps, typically ranging from 0.2 nM up to 1 μM) were injected over immobilized protein. Binding affinity and kinetics were measured at 25° C. with a flow rate of 100 μl/min in running buffer. Compounds were injected for 60 s followed by a dissociation time of up to 1000 s.

The double-referenced sensorgrams were fit to a simple reversible Langmuir 1:1 reaction mechanism as implemented in the Biacore® T200 and S200 evaluation software (T200 evaluation software version 2.0 and S200 evaluation software version 1.0, GE Healthcare).

TABLE 8
K_{D}, kₒₙ and k_{off} values (geometric mean values)
of MCL-1 compound interactions of selected examples
as determined in SPR assay (Assay 8)

| Example | kon [1/M 1/s] | koff [1/s] | KD [M] |
|---|---|---|---|
| 1 | | | |
| 2 | 1.1E7 | 6.8E−2 | 6.0E−9 |
| 3 | 2.8E5 | 4.6E−2 | 1.7E−7 |
| 4 | | | |
| 5 | | | |
| 6 | | | |
| 7 | | | |
| 8 | 6.2E6 | 2.1E−2 | 3.4E−9 |
| 9 | | | |
| 10 | | | |
| 11 | | | |
| 12 | | | |
| 13 | | | |
| 14 | | | |
| 15 | | | |
| 16 | | | |
| 17 | | | |
| 18 | | | |
| 19 | | | |
| 20 | | | |
| 21 | | | |
| 22 | | | |
| 23 | | | |
| 24 | | | |
| 25 | | | |
| 26 | | | |
| 27 | | | |
| 28 | | | |
| 29 | | | |
| 30 | | | |
| 31 | | | |
| 32 | | | |
| 33 | | | |
| 34 | 2.0E6 | 8.0E−3 | 4.0E−9 |
| 35 | 5.8E5 | 6.6E−2 | 1.1E−7 |
| 36 | | | |
| 37 | 2.4E6 | 1.1E−2 | 4.7E−9 |
| 38 | | | |
| 39 | | | |
| 40 | | | |
| 41 | | | |
| 42 | | | |
| 43 | 5.6E6 | 9.6E−3 | 1.7E−9 |
| 44 | | | |
| 45 | | | |
| 46 | | | |
| 47 | | | |
| 48 | 7.5E5 | 6.0E−3 | 8.0E−9 |
| 49 | 1.9E6 | 5.8E−3 | 3.0E−9 |
| 50 | 1.6E6 | 6.1E−3 | 3.8E−9 |
| 51 | | | |
| 52 | 1.6E7 | 2.4E−2 | 1.5E−9 |
| 53 | | | |
| 54 | | | |
| 55 | 1.0E6 | 4.0E−3 | 3.9E−9 |
| 56 | | | |
| 57 | | | |
| 58 | | | |
| 59 | | | |
| 60 | | | |
| 61 | | | |
| 62 | | | |
| 63 | | | |
| 64 | | | |
| 65 | 8.0E6 | 1.1E−2 | 1.4E−9 |
| 66 | | | |
| 67 | | | |
| 68 | | | |
| 69 | | | |
| 70 | | | |
| 71 | | | |
| 72 | | | |
| 73 | | | |
| 74 | | | |
| 75 | | | |
| 76 | | | |
| 77 | | | |
| 78 | | | |
| 79 | | | |
| 80 | | | |
| 81 | | | |
| 82 | | | |
| 83 | | | |
| 84 | | | |
| 85 | | | |
| 86 | | | |
| 87 | | | |
| 88 | | | |
| 89 | | | |
| 90 | | | |
| 91 | | | |
| 92 | | | |
| 93 | | | |
| 94 | | | |

Assay 9

Equilibrium Shake Flask Solubility Assay

Thermodynamic solubility can be determined by an equilibrium shake flask method [Edward H. Kerns and Li Di (2008) Solubility Methods in: Drug-like Properties: Concepts, Structure Design and Methods, p 276-286. Burlington, Mass.: Academic Press].

A saturated solution of the drug is prepared and the solution is mixed for 24 h to ensure that equilibrium has been reached. The solution is centrifuged to remove the insoluble fraction and the concentration of the compound in solution is determined using a standard calibration curve. To prepare the sample, 2 mg solid compound are weighed in a 4 mL glass vial. 1 mL phosphate buffer pH 6.5 respectively borate Buffer pH 8 is added. The suspension is put on a stirrer and mixed for 24 hrs at room temperature. The solution is centrifuged afterwards. To prepare the sample for the standard calibration, 1-2 mg (accurate weight) solid sample is dissolved in acetonitrile/water 50:50 and diluted to 20 mL. Sample and standards can be quantified by HPLC with UV-detection. For each sample two injection volumes (5 and 50 μL) in triplicates are made. Three injection volumes (5 μL, 10 μL and 20 μL) are made for the standard.

Chromatographic conditions are as follows:

HPLC column: Xterra MS C18 2.5 μm 4.6×30 mm

Injection volume: Sample: 3×5 μl and 3×50 μl
Standard: 5 μl, 10 μl, 20 μl

Flow: 1.5 mL/min

Mobile phase: acidic gradient:
A: Water/0.01% trifluoroacidic acid
B: Acetonitrile/0.01% trifluoroacidic acid
0 min→95% A 5% B
0-3 min→35% A 65% B, linear gradient
3-5 min→35% A 65% B, isocratic
5-6 min→95% A 5% B, isocratic

UV detector: wavelength near the absorption maximum (between 200 and 400 nm)

The areas of sample- and standard injections as well as the calculation of the solubility value (in mg/L) can be determined by using HPLC software (Waters Empower 2 FR).

Assay 10

Caco-2 Permeation Assay

Caco-2 cells (purchased from DSMZ Braunschweig, Germany) were seeded at a density of 4.5×10⁴ cell per well on 24 well insert plates, 0.4 μm pore size, and grown for 15 days in DMEM medium supplemented with 10% fetal bovine serum, 1% GlutaMAX (100×, GIBCO), 100 U/mL penicillin, 100 μg/mL streptomycin (GIBCO) and 1% non essential amino acids (100×). Cells were maintained at 37° C. in a humidified 5% CO₂ atmosphere. Medium was changed every 2-3 day. Before running the permeation assay, the culture medium was replaced by an FCS-free hepes-carbonate transport puffer (pH 7.2). For assessment of monolayer integrity the transepithelial electrical resistance (TEER) was measured. Test compounds were predissolved in DMSO and added either to the apical or basolateral compartment in final concentration of 2 μM. Before and after 2 h incubation at 37° C. samples were taken from both compartments. Analysis of compound content was done after precipitation with methanol by LC/MS/MS analysis. Permeability (Papp) was calculated in the apical to basolateral (A→B) and basolateral to apical (B→A) directions. The apparent permeability was calculated using following equation:

Pₐₚₚ=(Vᵣ/Pₒ)(1/S)(P₂/t)

Where Vᵣ is the volume of medium in the receiver chamber, Pₒ is the measured peak area of the test drug in the donor chamber at t=o, S the surface area of the monolayer, P₂ is the measured peak area of the test drug in the acceptor chamber after 2 h of incubation, and t is the incubation time. The efflux ratio basolateral (B) to apical (A) was calculated by dividing the Pₐₚₚ B-A by the Pₐₚₚ A-B. In addition the compound recovery was calculated. As assay control reference compounds were analyzed in parallel.

Assay 11

CYP Inhibition Assay

The inhibitory potency of the test compounds towards cytochrome P450 dependent metabolic pathways can be determined in human liver microsomes applying individual CYP isoform-selective standard probes (phenacetin, coumarin, bupropion, amodiaquine, diclofenac, S-mephenytoin, dextromethorphan, chlorzoxazone, midazolam, testosterone). Reference inhibitors are included as positive controls. Incubation conditions (protein and substrate concentration, incubation time) are optimized with regard to linearity of metabolite formation. The assay is processed by using Genesis Workstation (Tecan, Crailsheim, FRG) in 96-well plates at 37° C. After protein precipitation the metabolite formation is quantified by LC-MS/MS analysis followed by inhibition evaluation and IC₅₀ calculation.

The potential of an investigational drug to inhibit CYP enzymes, given by determined IC₅₀ values of test compounds in vitro, is a basic requirement in order to assess potential drug-drug interactions (DDI) with comedicated drugs which are relevant substrates of studied CYP isoforms. Such investigations are recommended by pertinent guidelines (i.e. EMA and FDA) for the evaluation of DDIs.

Assay 12

CYP Induction Assay

To evaluate the CYP induction potential in vitro, cultured human hepatocytes from three separate livers are treated once daily for three consecutive days with vehicle control, one of eight concentrations of test compound and known human CYP inducers (e.g. omeprazole, phenobarbital, and rifampin). After treatment, the cells are incubated in situ with the appropriate marker substrates for the analysis of CYP3A4, CYP2B6 and CYP1A2 activity by LC-MS/MS. Following the in situ incubation, the same hepatocytes from the same treatment groups are harvested for RNA isolation and analyzed by qRT-PCR to assess the effect of test compound on CYP1A2, CYP2B6 and CYP3A4 mRNA expression levels.

Assay 13

Investigation of In Vitro Metabolic Stability in Rat Hepatocytes (Including Calculation of Hepatic In Vivo Blood Clearance (CL))

Hepatocytes from Han Wistar rats were isolated via a 2-step perfusion method. After perfusion, the liver was carefully removed from the rat: the liver capsule was opened and the hepatocytes were gently shaken out into a Petri dish with ice-cold WME. The resulting cell suspension was filtered through sterile gaze in 50 mL falcon tubes and centrifuged at 50×g for 3 min at room temperature. The cell pellet was resuspended in 30 mL WME and centrifuged through a Percoll® gradient for 2 times at 100×g. The hepatocytes were washed again with Williams' medium E (WME) and resuspended in medium containing 5% FCS. Cell viability was determined by trypan blue exclusion.

For the metabolic stability assay liver cells were distributed in WME containing 5% FCS to glass vials at a density of 1.0×10⁶ vital cells/mL. The test compound was added to a final concentration of 1 μM. During incubation, the hepatocyte suspensions were continuously shaken and aliquots were taken at 2, 8, 16, 30, 45 and 90 min, to which equal volumes of cold methanol were immediately added. Samples were frozen at −20° C. over night, after subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, amount of liver cells in vivo and in vitro. The hepatic in vivo blood clearance (CLblood) and the maximal oral bioavailability (Fₘₐₓ) was calculated using the following formulae: CL′intrinsic [ml/(min*kg)]=kel [1/min]/((cellno/volume of incubation [ml])*fu,inc)*(cellno/liver weight [g])*(specific liver weight [g liver/kg body weight]); CLblood well-stirred [L/(h*kg)]=(QH [L/(h*kg)]*fu,blood*CL′intrinsic [L/(h*kg)])/(QH [L/(h*kg)]+fu,blood*CL′intrinsic [L/(h*kg)]); Fmax=1−CLblood/QH. The following parameter values were used: Liver blood flow—4.2 L/h/kg rat; specific liver weight—32 g/kg rat body weight; liver cells in vivo—1.1×10⁸ cells/g liver, liver cells in vitro—1.0×10⁶/ml; fu,inc and fu,blood is taken as 1.

Assay 14

Investigation of In Vitro Metabolic Stability in Rat Hepatocytes in Liver Microsomes (Including Calculation of Hepatic In Vivo Blood Clearance (CL) and of Maximal Oral Bioavailability (Fmax))

The in vitro metabolic stability of test compounds was determined by incubating them at 1 μM in a suspension liver microsomes in 100 mM phosphate buffer, pH7.4 (sodium dihydrogen phosphate monohydrate (NaH₂PO₄×H₂O)+disodium hydrogen phosphate dihydrate (Na₂HPO₄×2H₂O) and at a protein concentration of 0.5 mg/mL at 37° C. The microsomes were activated by adding a co-factor mix containing 8 mM Glukose-6-Phosphat, 4 mM magnesium chloride; 0.5 mM NADP and 1 IU/ml G-6-P-Dehydrogenase in phosphate buffer, pH 7.4. The metabolic assay was started shortly afterwards by adding the test compound to the incubation at a final volume of 1 mL. Organic solvent in the incubations was limited to ≤0.01% dimethylsulfoxide (DMSO) and ≤1% acetonitrile. During incubation, the microsomal suspensions were continuously shaken at 580 rpm and aliquots were taken at 2, 8, 16, 30, 45 and 60 min, to which equal volumes of cold methanol were immediately added. Samples were frozen at −20° C. over night, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, specific liver weight and microsomal protein content the hepatic in vivo blood clearance (CL) and the maximal oral bioavailability (Fₘₐₓ) were calculated for the different species. The hepatic in vivo blood clearance (CLblood) and the maximal oral bioavailability (Fₘₐₓ) was calculated using the following formulae: CL′intrinsic [ml/(min*kg)]=kel [1/min]/((mg protein/volume of incubation [ml])*fu,inc)*(mg protein/liver weight [g])*(specific liver weight [g liver/kg body weight]); CLblood well-stirred [L/(h*kg)]=(QH [L/(h*kg)]*fu,blood*CL′intrinsic [L/(h*kg)])/(QH [L/(h*kg)]+fu,blood*CL′intrinsic [L/(h*kg)]); Fmax=1−CLblood/QH and using the following parameter values: Liver blood flow—1.32 L/h/kg (human), 2.1 L/h/kg (dog), 4.2 L/h/kg (rat); specific liver weight—21 g/kg (human), 39 g/kg (dog), 32 g/kg (rat); microsomal protein content—40 mg/g.; fu,inc and fu,blood is taken as 1.

Assay 15

In Vivo Pharmacokinetics in Rats

For in vivo pharmacokinetic experiments test compounds were administered to male Wistar rats intravenously at doses of 0.3 to 1 mg/kg and intragastral at doses of 0.5 to 10 mg/kg formulated as solutions using solubilizers such as PEG400 in well-tolerated amounts.

For pharmacokinetics after intravenous administration test compounds were given as i.v. bolus and blood samples were taken at 2 min, 8 min, 15 min, 30 min, 45 min, 1 h, 2 h, 4 h, 6 h, 8 h and 24 h after dosing. Depending on the expected half-life additional samples were taken at later time points (e.g., 48 h, 72 h). For pharmacokinetics after intragastral administration test compounds were given intragastral to fasted rats and blood samples were taken at 5 min, 15 min, 30 min, 45 min, 1 h, 2 h, 4 h, 6 h, 8 h and 24 h after dosing. Depending on the expected half-life additional samples were taken at later time points (e.g., 48 h, 72 h). Blood was collected into Lithium-Heparintubes (Monovetten®, Sarstedt) and centrifuged for 15 min at 3000 rpm. An aliquot of 100 μL from the supernatant (plasma) was taken and precipitated by addition of 400 μL cold acetonitrile and frozen at −20° C. over night. Samples were subsequently thawed and centrifuged at 3000 rpm, 4° C. for 20 minutes. Aliquots of the supernatants were taken for analytical testing using an Agilent 1200 HPLC-system with LCMS/MS detection. PK parameters were calculated by non-compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. PK parameters calculated from concentration time profiles after i.g.: Cmax: Maximal plasma concentration (in mg/L); Cmaxnorm: Cmax divided by the administered dose (in kg/L); Tmax: Time point at which Cmax was observed (in h). Parameters calculated from both, i.v. and i.g. concentration-time profiles: AUCnorm: Area under the concentration-time curve from t=0 h to infinity (extrapolated) divided by the administered dose (in kg*h/L); AUC(0-tlast)norm: Area under the concentration-time curve from t=0 h to the last time point for which plasma concentrations could be measured divided by the administered dose (in kg*h/L); t½: terminal half-life (in h); F: oral bioavailability: AUCnorm after intragastral administration divided by AUCnorm after intravenous administration (in %).

The suitability of the compounds of the present invention for the treatment of hyperproliferative disorders can be demonstrated in animal models of the following cancer types: Lymphoma, Non-Hodgkin-Lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; melanoma; ovarian cancer; pancreas cancer For this purpose, human tumor cells of the respective cancer type are injected subcutaneously into immunocompromised mice. Once the primary tumor growth is established the animals will be then randomized to receive treatment with either compound at maximum tolerated dose or vehicle control for a certain period of time. The difference between those groups in terms of the tumor growth will be used to access the treatment efficacy. The principles of such xenograft studies are summarized in Richmond, A.; Su, Y. (2008). “Mouse xenograft models vs GEM models for human cancer therapeutics”. Disease Models and Mechanisms 1 (2-3): 78-82. doi:10.1242/dmm.000976.

Assay 16

In Vivo Pharmacokinetics in Mouse

For in vivo pharmacokinetic experiments test compounds are administered to female CD1 mouse intravenously at doses of 0.3 to 1 mg/kg formulated as solutions using solubilizers such as PEG400 in well-tolerated amounts.

For pharmacokinetics after intravenous administration test compounds are given as i.v. bolus and blood samples are taken at 2 min, 5 min, 15 min, 30 min, 1 h, 2 h, 4 h, 7 h, and 24 h after dosing. Blood is collected via a vena jugularis catheter into Lithium-Heparin coated tubes (Eppendorf) and centrifuged for 15 min at 3000 rpm. An aliquot from the supernatant (plasma) is taken and precipitated by addition of 1:10 (v/v) ice cold methanol and frozen at −20° C. over night. Samples are subsequently thawed and centrifuged at 3000 rpm, 4° C. for 20 minutes. Aliquots of the supernatants are taken for analytical testing using an Agilent 1200 HPLC-system with LCMS/MS detection. PK parameters are calculated by non- compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. AUCnorm: Area under the concentration-time curve from t=0 h to infinity (extrapolated) divided by the administered dose (in kg*h/L); AUC(0-tlast)norm: Area under the concentration-time curve from t=0 h to the last time point for which plasma concentrations could be measured divided by the administered dose (in kg*h/L); t½: terminal half-life (in h); MRT iv (h): mean residence time.

Assay 17

In Vivo Pharmacokinetics in Dog

For in vivo pharmacokinetic experiments test compounds are administered to Beagle dogs intravenously at doses of 0.3 to 1 mg/kg formulated as solutions using solubilizers such as PEG400 in well-tolerated amounts.

For pharmacokinetics after intravenous administration test compounds are given in dogs as short term infusion (10 min). Blood samples are taken e.g. at 5 min, 10 min (end of short term infusion), 15 min, 30 min, 1 h, 2 h, 4 h, 6 h, 8 h and 24 h after dosing from the vena saphena. Blood is collected into K-EDTA (Monovetten®, Sarstedt) and centrifuged for 15 min at 3000 rpm. An aliquot of 100 μL from the supernatant (plasma) is taken and precipitated by addition of 400 μL cold acetonitrile and frozen at −20° C. over night. Samples are subsequently thawed and centrifuged at 3000 rpm, 4° C. for 20 minutes. Aliquots of the supernatants are taken for analytical testing using an Agilent HPLC-system with LCMS/MS detection. PK parameters are calculated by non-compartmental analysis using a PK calculation software (e.g. Phoenix WinNonlin, Certara USA, Inc.).

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. AUCnorm: Area under the concentration-time curve from t=0 h to infinity (extrapolated) divided by the administered dose (in kg*h/L); AUC(0-tlast)norm: Area under the concentration-time curve from t=0 h to the last time point for which plasma concentrations could be measured divided by the administered dose (in kg*h/L); t½: terminal half-life (in h); MRT iv (h): mean residence time.

1. A compound of general formula (I): 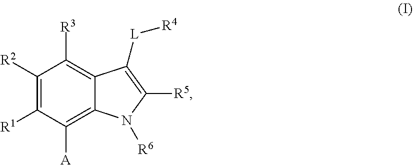
wherein
A is 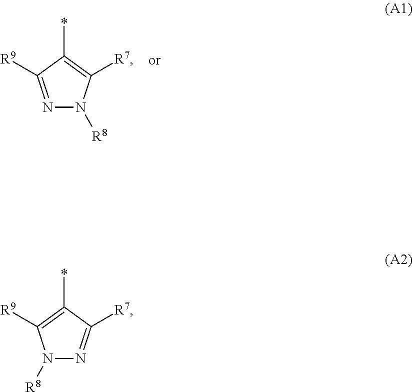
wherein R⁶ and R⁷, together with two carbon atoms of the pyrazole ring, two carbon atoms of the indole moiety and the nitrogen atom to which R⁶ is attached, form a 9- to 16-membered ring and * is the point of attachment of these moieties to the indole carbon atom bearing the A substituent
or
A is 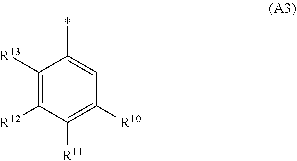
wherein optionally one or two of the groups selected from CR¹¹, CR¹² and CR¹³ are replaced by a nitrogen atom,
wherein R⁶ and R¹⁰, together with three carbon atoms of the phenyl ring, two carbon atoms of the indole moiety and the nitrogen atom to which R⁶ is attached, form a 9- to 16-membered ring and * is the point of attachment of these moieties to the indole carbon atom bearing the A substituent;
R¹ and R² are each independently selected from a hydrogen atom, a halogen atom, a cyano group, a C₁-C₃-alkyl group, and a C₁-C₃-alkoxy group;
R³ is selected from a hydrogen atom, a halogen atom, a cyano group, a C₁-C₃-alkyl group, a C₁-C₃-haloalkyl group, a C₁-C₃-alkoxy group, a C₁-C₃-alkylthio group, a —S(O)—(C₁-C₃-alkyl) group, a —S(O)₂—(C₁-C₃-alkyl) group, a C₁-C₃-haloalkoxy group, a C₁-C₃-haloalkylthio group, and a C₃-C₅-cycloalkyl group;
R⁴ is selected from an aryl group and a heteroaryl group, each of which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group, a C₁-C₃-thioalkyl group, a C₁-C₃-haloalkoxy group, a (C₁-C₃)-haloalkyl-S— group, and a C₃-C₅-cycloalkyl group;
L is a group —(CH₂)ₘ-E- wherein any CH₂ group is unsubstituted or substituted with one or two substituents and each substituent is independently selected from a halogen atom, a cyano group, a hydroxyl group, a C₁-C₃-alkyl group, and a C₁-C₃-alkoxy group, or two substituents are optionally taken together with their intervening atoms to form a saturated or partially unsaturated 3-6-membered cycloalkyl ring, or a 3-8 membered saturated or partially unsaturated heterocyclic ring having 1-2 heteroatoms independently selected from an oxygen atom, a sulfur atom, a —S(O)— group, a —S(O)₂— group, and a —NR¹⁴— group;
E is a bond, an oxygen atom, a sulfur atom, a —S(O)— group, a —S(O)₂— group or a —NR¹⁴— group and constitutes the connecting element to R⁴,
m is 2, 3, or 4;
R⁵ is selected from a COOH group, a 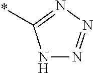 [SMILES: *c1nnn[nH]1]
group, a —C(O)—NHS(O)₂(C₁-C₆-alkyl) group, a —C(O)—NHS(O)₂(C₃-C₆-cycloalkyl) group, a —C(O)—NHS(O)₂(aryl) group, a —C(O)—NHS(O)₂(CH₂)ₛNHC(C₁-C₆-alkyl) group, a —C(O)—NHS(O)₂(CH₂)ₛNHCO(C₃-C₆-cycloalkyl) group, and a —C(O)—NHS(O)₂(CH₂)ₛNHCO(aryl) group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, a C₁-C₃-alkoxy group, a C₁-C₃-haloalkoxy group, and a (heterocycloalkyl)-(C₁-C₃-alkylene)- group, and where a —CH═CH— group in any alkenylene group can be replaced by a 1,2-cyclopropylene group, and said 1,2-cyclopropylene group is unsubstituted or substituted one or two times with a halogen atom or a C₁-C₂-alkyl group, wherein ^{#} is the point of attachment with the indole nitrogen atom and ^{##} is the point of attachment with the pyrazole carbon atom bearing the R⁷ substituent;
—R⁶-R¹⁰— is ^{#}—(CH₂)ₙ-(G)-(CH₂)ᵣ—(B)ₜ—(CH₂)ₚ—^{##}, where one or more CH₂ groups are unsubstituted or substituted with one or more substituents selected from a halogen atom, a hydroxyl group, a NR¹⁷R¹⁸ group, a C₁-C₃-alkyl group, a C₁-C₃-haloalkyl group, a C₁-C₃-alkoxy group, a C₁-C₃-haloalkoxy group and a (heterocycloalkyl)-(C₁-C₃-alkylene)- group, wherein ^{#} is the point of attachment with the indole nitrogen atom and ^{##} is the point of attachment with the carbon atom of the phenyl moiety bearing the R¹⁰ substituent;
n is 1, 2, 3, 4, 5, 6, 7, 8, 9, or 10;
t is 0 or 1;
r is 0, 1, 2, or 3;
s is 0, 1, 2, or 3;
p is 0, 1, 2, 3, 4, 5, or 6;
where the integers selected for variables n, t, r, s, and p result in forming a 9- to 16-membered ring independently from the selection of variable A1, A2 or A3;
B is independently selected from a —C(O)NR¹⁵— group, a —NR¹⁵C(O)— group, a —N(R¹⁵)— group, a —N(R¹⁵)—C(═O)—N(R¹⁵)— group, a —O—C(═O)—N(R¹⁵)— group, a —N(R¹⁵)—C(═O)—O— group, —O—, —S—, —S(O)—, —S(O)₂—, a —S(O)NR¹⁵— group, a —NR¹⁵S(O)— group, a —S(O)₂NR¹⁵— group, a —NR¹⁵S(O)₂— group, a 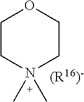
group and a —[N⁺(R²¹R²²)—(R¹⁶)⁻]— group,
G is a 1,2-arylene group or
a mono- or bicyclic heteroarylene group wherein two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group;
R⁸ is selected from a hydrogen atom,
a C₁-C₆-alkyl group, which is unsubstituted or substituted with one or more substituents independently selected from
a halogen atom, a hydroxyl group, a C₁-C₃-alkoxy group, a C₁-C₃-haloalkoxy group, a C₃-C₆-cycloalkyl group, a heterocycloalkyl group, and a NR²¹R²² group, or
a C₁-C₃-haloalkyl group, a C₃-C₆-cycloalkyl group, or a C₁-C₆-alkyl group in which one or two not directly adjacent carbon atoms are independently replaced by a heteroatom selected from —O— and —NH—,
R⁹ is selected from a hydrogen atom,
a C₁-C₄-alkyl group,
a C₁-C₃-hydroxyalkyl group,
a C₁-C₄-haloalkyl group,
a C₁-C₄-haloalkyl-NH—C(O)—O—(C₁-C₃-alkylene)- group,
a C₂-C₆-haloalkenyl group,
a C₁-C₆-alkyl-O— group,
a C₁-C₄-haloalkoxy group,
a C₁-C₆-alkyl-O—(C₁-C₃-alkylene)- group,
a (C₃-C₇)-cycloalkyl group,
a (C₃-C₇)-cycloalkyl-O—(C₁-C₃-alkylene)- group,
a phenyl-O—(C₁-C₃-alkylene)- group,
a phenyl-(C₁-C₃-alkylene)-O—(C₁-C₃-alkylene)- group,
a R¹⁹-(phenylene)-(C₁-C₃-alkylene)-O—(C₁-C₃-alkylene)- group,
a R¹⁹-(phenylene)-O—(C₁-C₃-alkylene)- group,
a R¹⁹-(phenylene)-(heteroarylene)-O—(C₁-C₃-alkylene) group,
a (R¹⁹)-(heterocycloalkylene)-(C₁-C₃-alkylene)- group,
a (R¹⁹)-(heterocycloalkylene)-(phenylene)-O—(C₁-C₃-alkylene)- group,
a (heterocycloalkenyl)-(phenylene)-O—(C₁-C₃-alkylene)- group,
a (R¹⁹)-(heteroarylene)-(C₁-C₃-alkylene)-O—(C₁-C₃-alkylene)- group,
a (R¹⁹)-(heteroarylene)-(phenylene)-O—(C₁-C₃-alkylene)- group,
a (R²⁰)—S(O)₂-(phenylene)-O—(C₁-C₃-alkylene)- group,
a (R²⁰)—S(O)₂—NH-(phenylene)-O—(C₁-C₃-alkylene)- group,
a (R²⁰)—S(O)₂—NH-(phenylene)-O—(C₁-C₃-alkylene)- group,
a (R²⁰)—S(O)₂—N(C₁-C₆-alkyl)-(phenylene)-O—(C₁-C₃-alkylene)- group,
a (R²⁰)—S(O)₂-(heterocycloalkylene)-(phenylene)-O—(C₁-C₃-alkylene)- group,
a (R¹⁹)-(heterocycloalkylene)-(heteroarylene)-O—(C₁-C₃-alkylene)- group,
a (R²⁰)-(heteroarylene)-O—(C₁-C₃-alkylene)- group,
a (R¹⁹)-(heteroarylene)-O—(C₁-C₃-alkylene)- group,
a (R²⁰)—S(O)₂-(heteroarylene)-O—(C₁-C₃-alkylene)- group,
a (R²⁰)—S(O)₂-(heterocycloalkylene)-(heteroarylene)-O—(C₁-C₃-alkylene)- group,
a NR²¹R²²—(C₁-C₃-alkylene)- group,
a (C₁-C₃-alkyl)-NH—(C₁-C₃-alkylene)- group,
a (C₁-C₃-haloalkyl)-(C₁-C₃-alkylene)-NH—(C₁-C₃-alkylene)- group,
a (C₁-C₃-haloalkyl)-NH—(C₁-C₃-alkylene)- group,
a (C₁-C₃-alkyl)-NH—C(O)—(C₁-C₃-alkylene)- group,
a (C₁-C₃-alkyl)-NR¹⁵—C(O)—(C₁-C₃-alkylene)- group,
a (C₁-C₃-alkyl)-C(O)—NH—(C₁-C₃-alkylene)- group,
a (C₁-C₃-alkyl)-C(O)—NR¹⁵—(C₁-C₃-alkylene)- group,
a 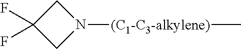
group, and a 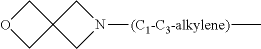
group, where the phenyl ring is unsubstituted or substituted with a halogen atom, a hydroxyl group, or a C₁-C₃-alkoxy group and
the heterocycloalkyl group is unsubstituted or substituted with an oxo (═O) group or is unsubstituted or substituted with one or more substituents independently selected from a halogen atom, a hydroxyl group, a C₁-C₃-alkyl group, and a C₁-C₃-alkoxy group,
or R⁸ and R⁹ together form a 5- or 6-membered ring optionally comprising one or two heteroatoms independently selected from —O— and —NR¹⁴—;
R¹¹ and R¹³ are each independently selected from a hydrogen atom, a halogen atom, a C₁-C₃-alkyl group, and a C₁-C₃-alkoxy group;
R¹² is selected from a hydrogen atom, a C₁-C₃-alkoxy group, a C₁-C₃-alkyl group, a C₁-C₃-haloalkyl group, a C₁-C₃-haloalkoxy group, and a NR¹⁷R¹⁸ group;
R¹⁴ is a hydrogen atom or a C₁-C₃-alkyl group;
R¹⁵ is independently selected from a hydrogen atom,
a C₁-C₆-alkyl group
which is unsubstituted or substituted with one or more substituents selected from a halogen atom, a C₁-C₃-alkyl group, a C₁-C₃-haloalkyl group, a C₁-C₃-hydroxyalkyl group, a C₁-C₃-alkoxy group, a C₁-C₃-haloalkoxy group, a heterocycloalkyl group, an aryl group, a (R¹⁹)-(heterocycloalkylene)-(arylene)-O— group, a (heterocycloalkyl)-(arylene)-O— group, an aryl-O— group, an aryl-(C₁-C₃-alkylene)-O— group, a (R²⁰)—S(O)₂-arylene-O— group, a (R²⁰)S(O)₂-heterocycloalkylene-arylene-O— group, an aryl-heteroarylene-O— group, an aryl-heteroarylene-O—(C₁-C₃-alkylene)- group, a heterocycloalkyl-(C₁-C₃-alkylene)-C(O)— group, a heterocycloalkyl-NH—C(O)— group, an aryl-(C₁-C₃-alkylene)-NH—C(O)— group, a heterocycloalkylene-(C₁-C₃-alkylene)-S(O)₂— group, and a heterocycloalkylene-heteroarylene-S(O)₂— group;
a C₁-C₃-alkylene-C(O)— group,
a heterocycloalkyl-(C₁-C₃-alkylene)-C(O)— group,
a heterocycloalkyl-(C₁-C₃-alkylene)-S(O)₂— group,
a heterocyclyl-NH—C(O)— group,
a heterocycloalkyl-(C₁-C₃-alkylene)-NH—C(O)— group,
an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group,
a heterocycloalkyl-heteroarylene-S(O)₂— group,
a phenyl group,
a group 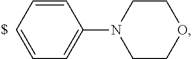
a group 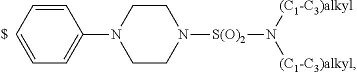
and
a group 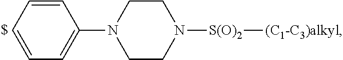
where $ is the point of attachment to the nitrogen atom, to which R¹⁵ is attached;
R¹⁶ is a pharmaceutically acceptable anion;
R¹⁷ and R¹⁸ are each independently selected from a hydrogen atom, a C₁-C₆-alkyl group, a C₁-C₆-haloalkyl group, a C₃-C₅-cycloalkyl group, a C₁-C₃-alkyl-C(O)— group, a C₁-C₃-alkylS(O)₂— group, and a C₁-C₃-alkyl-O—C(═O)— group;
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
R²⁰ is selected from a C₁-C₃-alkyl group, a C₃-C₆-cycloalkyl group, and a NR²¹R²² group; and
R²¹ and R²² are independently selected from a hydrogen atom or a C₁-C₆-alkyl group;
or a tautomer, an N-oxide, or a salt thereof, or a salt of a tautomer, or a salt of an N-oxide, or a mixture of same.

2. The compound of general formula (I) according to claim 1
wherein
A is 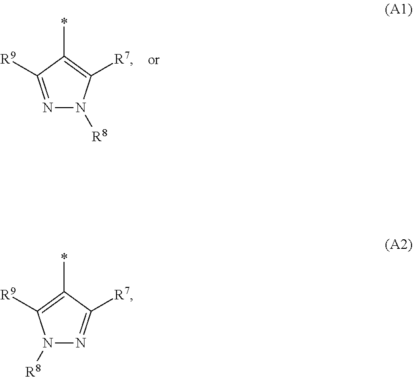
wherein R⁶ and R⁷, together with two carbon atoms of the pyrazole ring, two carbon atoms of the indole moiety and the nitrogen atom to which R⁶ is attached, form a 9- to 13-membered ring and * is the point of attachment of these moieties to the indole carbon atom bearing the A substituent;
R¹ and R² are each independently selected from a hydrogen atom, a halogen atom, a cyano group, a C₁-C₃-alkyl group, and a C₁-C₃-alkoxy group;
R³ is selected from a hydrogen atom, a halogen atom, a cyano group, a C₁-C₃-alkyl group, a C₁-C₃-haloalkyl group, a C₁-C₃-alkoxy group and a C₃-C₅-cycloalkyl group;
R⁴ is selected from an aryl group and a heteroaryl group, each of which is unsubstituted or substituted with one, two, three, four or five substituents and each substituent is independently selected from a halogen atom, a cyano group, a C₁-C₃-alkyl group, a C₁-C₃-haloalkyl group, a C₁-C₃-alkoxy group and a C₃-C₅-cycloalkyl group;
L is a group —(CH₂)ₘ-E- wherein any CH₂ group is unsubstituted or substituted with one or two substituents and each substituent is independently selected from a halogen atom, a cyano group, a hydroxyl group, a C₁-C₃-alkyl group and a C₁-C₃-alkoxy group;
E is a bond, an oxygen atom, a sulfur atom, or a —NR¹⁴— group and constitutes the connecting element to R⁴,
m is 2, 3, or 4;
R⁵ is selected from a COOH group and a 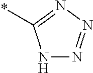 [SMILES: *c1nnn[nH]1]
group;
—R⁶-R⁷— is ^{#}—(CH₂)ₙ-(G)-(CH₂)ᵣ—(B)ₜ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, and a C₁-C₃-alkoxy group, wherein ^{#} is the point of attachment with the indole nitrogen atom and ^{##} is the point of attachment with the pyrazole carbon atom bearing the R⁷ substituent;
n is 1, 2, or 3;
t is 1;
r is 1, 2, or 3;
p is 1, 2, or 3;
where the integers selected for variables n, t, r, and p result in forming a 9- to 13-membered ring independently from the selection of variable A1 or A2;
B is independently selected from a —C(O)NR¹⁵— group, a —NR¹⁵C(O)— group, a —N(R¹⁵)— group, and —O—;
G is a 1,2-arylene group or
a mono- or bicyclic heteroarylene group wherein two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group;
R⁸ is selected from a hydrogen atom, and
a C₁-C₆-alkyl group, which is unsubstituted or substituted with one or more substituents independently selected from
a halogen atom, a hydroxy group, a C₁-C₃-alkoxy group, a C₁-C₃-haloalkoxy group, a C₃-C₆-cycloalkyl group, a heterocycloalkyl group, and a NR²¹R²² group,
R⁹ is selected from a hydrogen atom,
a C₁-C₄-alkyl group,
a C₁-C₃-hydroxyalkyl group,
a C₁-C₄-haloalkyl group,
a C₁-C₄-haloalkyl-NH—C(O)—O—(C₁-C₃-alkylene)- group,
a C₂-C₆-haloalkenyl group,
a C₁-C₆-alkyl-O— group,
a C₁-C₄-haloalkoxy group,
a C₁-C₆-alkyl-O—(C₁-C₃-alkylene)- group,
a (C₃-C₇)-cycloalkyl group,
a (C₃-C₇)-cycloalkyl-O—(C₁-C₃-alkylene)- group,
a phenyl-O—(C₁-C₃-alkylene)- group,
a phenyl-(C₁-C₃-alkylene)-O—(C₁-C₃-alkylene)- group,
a R¹⁹-(phenylene)-(C₁-C₃-alkylene)-O—(C₁-C₃-alkylene)- group,
a R¹⁹-(phenylene)-O—(C₁-C₃-alkylene)- group,
a R¹⁹-(phenylene)-(heteroarylene)-O—(C₁-C₃-alkylene)- group,
a (R¹⁹)-(heterocycloalkylene)-(C₁-C₃-alkylene)- group,
a (R¹⁹)-(heterocycloalkylene)-(phenylene)-O—(C₁-C₃-alkylene)- group,
a (heterocycloalkenyl)-(phenylene)-O—(C₁-C₃-alkylene)- group,
a (R¹⁹)-(heteroarylene)-(C₁-C₃-alkylene)-O—(C₁-C₃-alkylene)- group,
a (R¹⁹)-(heteroarylene)-(phenylene)-O—(C₁-C₃-alkylene)- group,
a (R²⁰)—S(O)₂-(phenylene)-O—(C₁-C₃-alkylene)- group,
a (R²⁰)—S(O)₂—NH-(phenylene)-O—(C₁-C₃-alkylene)- group,
a (R²⁰)—S(O)₂—NH-(phenylene)-O—(C₁-C₃-alkylene)- group,
a (R²⁰)—S(O)₂—N(C₁-C₆-alkyl)-(phenylene)-O—(C₁-C₃-alkylene)- group,
a (R²⁰)—S(O)₂-(heterocycloalkylene)-(phenylene)-O—(C₁-C₃-alkylene)- group,
a (R¹⁹)-(heterocycloalkylene)-(heteroarylene)-O—(C₁-C₃-alkylene)- group,
a (R²⁰)-(heteroarylene)-O—(C₁-C₃-alkylene)- group,
a (R¹⁹)-(heteroarylene)-O—(C₁-C₃-alkylene)- group,
a (R²⁰)—S(O)₂-(heteroarylene)-O—(C₁-C₃-alkylene)- group,
a (R²⁰)—S(O)₂-(heterocycloalkylene)-(heteroarylene)-O—(C₁-C₃-alkylene)- group,
a NR²¹R²²—(C₁-C₃-alkylene)- group,
a (C₁-C₃-alkyl)-NH—(C₁-C₃-alkylene)- group,
a (C₁-C₃-haloalkyl)-(C₁-C₃-alkylene)-NH—(C₁-C₃-alkylene)- group,
a (C₁-C₃-haloalkyl)-NH—(C₁-C₃-alkylene)- group,
a (C₁-C₃-alkyl)-NH—C(O)—(C₁-C₃-alkylene)- group,
a (C₁-C₃-alkyl)-NR¹⁵—C(O)—(C₁-C₃-alkylene)- group,
a (C₁-C₃-alkyl)-C(O)—NH—(C₁-C₃-alkylene)- group,
a (C₁-C₃-alkyl)-C(O)—NR¹⁵—(C₁-C₃-alkylene)- group,
a 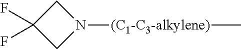
group, and a 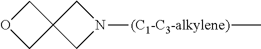
group, where the phenyl ring is unsubstituted or substituted with a halogen atom, a hydroxyl group, or a C₁-C₃-alkoxy group and
the heterocycloalkyl group is unsubstituted or substituted with an oxo (═O) group or is unsubstituted or substituted with one or more substituents independently selected from a halogen atom, a hydroxyl group, a C₁-C₃-alkyl group, and a C₁-C₃-alkoxy group,
R¹⁴ is a hydrogen atom or a C₁-C₃-alkyl group;
R¹⁵ is independently selected from a hydrogen atom,
a C₁-C₆-alkyl group
which is unsubstituted or substituted with one or more substituents selected from a C₁-C₃-alkyl group, a heterocycloalkyl group, and an aryl group;
a C₁-C₃-alkylene-C(O)— group,
a heterocycloalkyl-(C₁-C₃-alkylene)-C(O)— group,
a heterocycloalkyl-(C₁-C₃-alkylene)-S(O)₂— group,
a heterocycloalkyl-NH—C(O)— group,
a heterocycloalkyl-(C₁-C₃-alkylene)-NH—C(O)—,
an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group,
a heterocycloalkyl-heteroarylene-S(O)₂— group,
a phenyl group,
a group 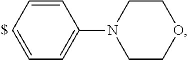
a group 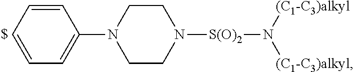
and
a group 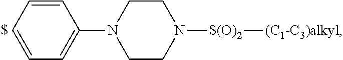
where $ is the point of attachment to the nitrogen atom, to which R¹⁵ is attached,
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
R²⁰ is selected from a C₁-C₃-alkyl group, a C₃-C₆-cycloalkyl group, and a NR²¹R²² group; and
R²¹ and R²² are independently selected from a hydrogen atom and a C₁-C₆-alkyl group;
or a tautomer, an N-oxide, or a salt thereof, or a salt of a tautomer, or a salt of an N-oxide, or a mixture of same.

3. The compound of general formula (I) according to claim 1
wherein
A is 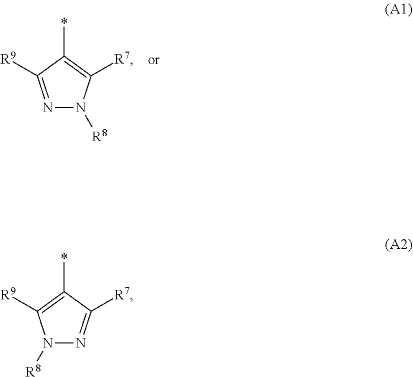
wherein R⁶ and R⁷, together with two carbon atoms of the pyrazole ring, two carbon atoms of the indole moiety and the nitrogen atom to which R⁶ is attached, form a 10- to 12-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three, substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 2, 3, or 4;
R⁵ is a COOH group;
—R⁶-R⁷— is ^{#}—(CH₂)ₙ-(G)-(CH₂)ᵣ—(B)ᵣ—(CH₂)ₚ—^{##}, wherein any —CH₂— group is unsubstituted or substituted with one or more substituents selected from a halogen atom, a hydroxyl group, a NR¹⁷R¹⁸ group, a C₁-C₃-alkyl group, a C₁-C₃-haloalkyl group, and a C₁-C₃-alkoxy group, wherein ^{#} is the point of attachment with the indole nitrogen atom and ^{##} is the point of attachment with the pyrazole carbon atom bearing the R⁷ substituent;
n is 1 or 2;
t is 1;
r is 1 or 2;
p is 1 or 2;
where the integers selected for variables n, t, r, and p result in forming a 10- to 12-membered ring independently from the selection of variable A1 or A2;
B is selected from a —N(R¹⁵)— group and —O—,
G is a 1,2-arylene group or
a monocyclic heteroarylene group wherein two vicinal carbon atoms thereof are each bound to one of the adjacent alkylene groups,
which are unsubstituted or substituted with one or more substituents independently selected from a halogen atom, a cyano group, a C₁-C₃-alkyl group, and a C₁-C₃-alkoxy group;
R⁸ is selected from a hydrogen atom, and
a C₁-C₆-alkyl group, which is unsubstituted or substituted with one or more substituents independently selected from
a halogen atom, a hydroxy group, a C₃-C₆-cycloalkyl group and a heterocycloalkyl group;
R⁹ is selected from a hydrogen atom,
a C₁-C₄-alkyl group,
a C₁-C₃-hydroxyalkyl group,
a C₁-C₄-haloalkyl group,
a C₁-C₆-alkyl-O— group,
a C₁-C₄-haloalkoxy group,
a C₁-C₆-alkyl-O—(C₁-C₃-alkylene)- group,
a (C₃-C₇)-cycloalkyl group,
a R¹⁹-(phenylene)-O—(C₁-C₃-alkylene)- group,
a NR²¹R²²—(C₁-C₃-alkylene)- group, and
a (C₁-C₃-alkyl)-NH—(C₁-C₃-alkylene)- group;
R¹⁵ is independently selected from a hydrogen atom,
a C₁-C₆-alkyl group,
a heterocycloalkyl-(C₁-C₃-alkylene)-C(O)— group,
a heterocycloalkyl-(C₁-C₃-alkylene)-S(O)₂— group,
a heterocycloalkyl-NH—C(O)— group,
a heterocycloalkyl-(C₁-C₃-alkylene)-NH—C(O)— group,
an aryl-(C₁-C₃-alkylene)-NH—C(O)— group, which is unsubstituted or substituted with 1, 2, or 3 substituents independently selected from a halogen atom, a C₁-C₃-alkyl group and a C₁-C₃-alkoxy group, and
a heterocycloalkyl-heteroarylene-S(O)₂— group;
R¹⁹ is selected from a hydrogen atom, a hydroxyl group, a cyano group, a C₁-C₃-alkyl group, a C₁-C₆-hydroxyalkyl group, a C₁-C₃-alkoxy group, a R²¹OC(O)—(C₁-C₃-alkylene)- group, a —C(O)OR²¹ group, a —C(O)NR²¹R²² group, a (C₁-C₃-alkyl)-O—(C₁-C₃-alkylene)-C(O)— group, a (C₁-C₆-alkyl)-C(O)— group, and a C₃-C₆-cycloalkyl-C(O)— group;
and
R²¹ and R²² are independently selected from a hydrogen atom and a C₁-C₆-alkyl group;
or a tautomer, an N-oxide, or a salt thereof, or a salt of a tautomer, or a salt of an N-oxide, or a mixture of same.

4. The compound of general formula (I) according to claim 1,
wherein
A is 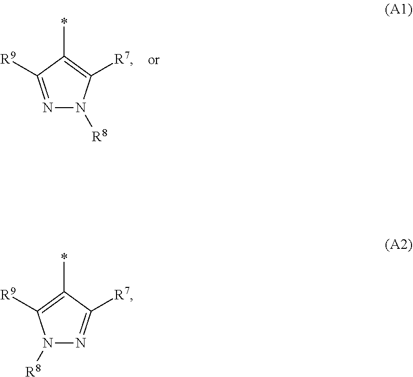
wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom and a halogen atom;
R³ is a hydrogen atom;
R⁴ is selected from an aryl group and a heteroaryl group, each of which is unsubstituted or substituted with one, two, or three substituents and each substituent is independently selected from a halogen atom, and a C₁-C₃-alkyl group;
L is a group —(CH₂)ₘ-E-;
E is a bond or an oxygen atom and constitutes the connecting element to R⁴;
m is 3;
R⁵ is a COOH group;
—R⁶-R⁷— is ^{#}—(CH₂)ₙ-(G)-(CH₂)ᵣ—(B)ₜ—(CH₂)ₚ—^{##}, wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p result in forming a 11-membered ring independently from the selection of variable A1 or A2;
B is independently selected from a —N(R¹⁵)— group and —O—;
G is a 1,2-arylene group or
a monocyclic heteroarylene group wherein two vicinal carbon atoms thereof are each bound to one of the adjacent alkylene groups,
which are each independently unsubstituted or substituted with one or more substituents selected from a halogen atom and a C₁-C₃-alkyl group;
R⁸ is selected from a hydrogen atom, and
a C₁-C₆-alkyl group, which is unsubstituted or substituted with a heterocycloalkyl group;
R⁹ is selected from a hydrogen atom,
a C₁-C₄-alkyl group,
a C₁-C₆-alkyl-O—(C₁-C₃-alkylene)- group,
R¹⁵ is independently selected from a hydrogen atom,
a C₁-C₆-alkyl group,
a heterocycloalkyl-(C₁-C₃-alkylene)-C(O)— group,
a heterocycloalkyl-(C₁-C₃-alkylene)-S(O)₂— group,
a heterocycloalkyl-NH—C(O)— group,
a heterocycloalkyl-(C₁-C₃-alkylene)-NH—C(O)— group,
a heterocycloalkyl-heteroarylene-S(O)₂— group, and
an aryl-(C₁-C₃-alkylene)-NH—C(O)— group, which unsubstituted or substituted with 1, 2, or 3 substituents independently selected from a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

5. The compound of general formula (I) according to claim 1,
wherein
A is 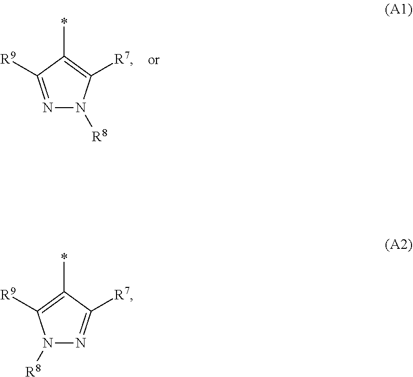
wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from an aryl group and a heteroaryl group, each of which is unsubstituted or substituted with one, two, or three, substituents and each substituent is independently selected from a halogen atom and a C₁-C₃-alkyl group;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p, result in forming a 11-membered ring independently from the selection of variable A1, or A2;
B is independently selected from a —N(R¹⁵)— group and —O—;
G is an 1,2-arylene group or
a monocyclic heteroarylene group whereby two vicinal carbon atoms thereof are each bound to one of the adjacent alkylene groups, and which each are unsubstituted;
R⁸ is selected from a hydrogen atom, a methyl group, and a —CH₂—CH₂—(N-morpholino) group;
R⁹ is selected from a hydrogen atom and a C₁-C₃-alkyl group;
R¹⁵ is a hydrogen atom,
a C₁-C₃-alkyl group,
a heterocycloalkyl-(C₁-C₃-alkylene)-C(O)— group,
a heterocycloalkyl-(C₁-C₃-alkylene)-S(O)₂— group,
a heterocycloalkyl-NH—C(O)— group,
a heterocycloalkyl-(C₁-C₃-alkylene)-NH—C(O)— group,
a heterocycloalkyl-heteroarylene-S(O)₂— group, and
an aryl-(C₁-C₃-alkylene)-NH—C(O)— group, which unsubstituted or substituted with 1, 2, or 3 substituents independently selected form a halogen atom, a C₁-C₃-alkyl group and a C₁-C₃-alkoxy group;
or a tautomer, an N-oxide, or a salt thereof, or a salt of a tautomer, or a salt of an N-oxide, or a mixture of same.

6. The compound of general formula (I) according to claim 1,
wherein
A is 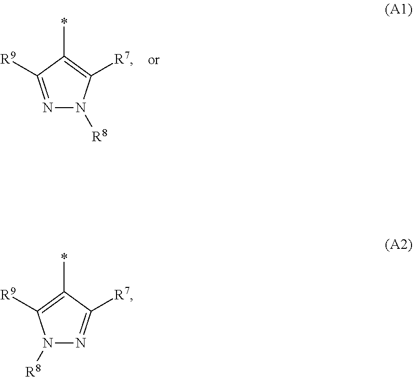
wherein R⁶ and R⁷, together with two carbon atoms of the pyrazole ring, two carbon atoms of the indole moiety and the nitrogen atom to which R⁶ is attached, form a 11-membered ring and * is the point of attachment of these moieties to the indole carbon atom bearing the A substituent;
R¹ and R² are each independently selected from a hydrogen atom, a fluorine atom and a chlorine atom;
R³ is a hydrogen atom;
R⁴ is selected from an aryl group and a heteroaryl group, each of which is unsubstituted or substituted with one, two, or three, substituents and each substituent is independently selected from a halogen atom and a C₁-C₃-alkyl group;
L is a group —(CH₂)₃—O—;
R⁵ is a COOH group;
—R⁶-R⁷— is ^{#}—(CH₂)ₙ-(G)-(CH₂)ᵣ—(B)ₜ—(CH₂)ₚ—^{##} wherein ^{#} is the point of attachment with the indole nitrogen atom and ^{##} is the point of attachment with the pyrazole carbon atom bearing the R⁷ substituent;
n is 1;
t is 1;
r is 1;
p is 1;
where the integers selected for variables n, t, r, and p, result in forming a 11-membered ring independently from the selection of variable A1, or A2;
B is independently selected from a —N(R¹⁵)— group and —O—;
G is an 1,2-arylene group or
a monocyclic heteroarylene group having 5 or 6 ring atoms which contains at least one heteroatom and wherein two vicinal carbon atoms thereof are each bound to one of the adjacent alkylene groups, and which each are unsubstituted;
R⁸ is selected from a hydrogen atom, a methyl group, and a —CH₂—CH₂—(N-morpholino) group;
R⁹ is selected from a hydrogen atom and a C₁-C₃-alkyl group;
R¹⁵ is a hydrogen atom,
a C₁-C₃-alkyl group,
a heterocycloalkyl-(C₁-C₃-alkylene)-C(O)— group,
a heterocycloalkyl-(C₁-C₃-alkylene)-S(O)₂— group,
a heterocycloalkyl-NH—C(O)— group,
a heterocycloalkyl-(C₁-C₃-alkylene)-NH—C(O)— group,
a heterocycloalkyl-heteroarylene-S(O)₂— group, and
an aryl-(C₁-C₃-alkylene)-NH—C(O)— group, which unsubstituted or substituted with 1, 2, or 3 substituents independently selected form a halogen atom, a C₁-C₃-alkyl group and/or a C₁-C₃-alkoxy group;
or a tautomer, an N-oxide, or a salt thereof, or a salt of a tautomer, or a salt of an N-oxide, or a mixture of same.

7. The compound of general formula (I) according to claim 1, which is selected from
(rac)-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid,
4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid-N-ethylethanamine salt (enantiomer 1),
4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid,
4,5-dimethyl-17-[3-(naphthalen-1-yloxy)propyl]-5,7,9,14-tetrahydropyrazolo[4′,3′:9,10]-pyrido[3′,2′:3,4][1,6]oxazacycloundecino[8,7,6-hi]indole-16-carboxylic acid (enantiomer 1),
4,5-dimethyl-17-[3-(naphthalen-1-yloxy)propyl]-5,7,9,14-tetrahydropyrazolo[4′,3′:9,10]-pyrido[3′,2′:3,4][1,6]oxazacycloundecino[8,7,6-hi]indole-16-carboxylic acid (enantiomer 2),
(rac)-4,5-dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid,
4,5-dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid-N-ethylethanamine salt (enantiomer 1),
4,5-dimethyl-17-[3-(naphthalen-1-yloxy)propyl]-5,7,9,14-tetrahydropyrazolo[4′,3′:9,10]-pyrido[2′,3′:3,4][1,6]oxazacycloundecino[8,7,6-hi]indole-16-carboxylic acid-N-ethylethanamine salt (enantiomer 2),
(rac)-4,5-dimethyl-19-[3-(naphthalen-1-yloxy)propyl]-5,7,9,16-tetrahydroindolo[1′,7′:6,7,8]-pyrazolo[4′,3′:9,10][1,6]oxazacycloundecino[3,4-b]quinoxaline-18-carboxylic acid,
(rac)-4,5-dimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-7,8,9,14-tetrahydro-5H-indolo[1,7-bc]pyrazolo-[4,3-e][2,8]benzodiazacycloundecine-16-carboxylic acid trifluoroacetate salt (enantiomer 1),
4,5-dimethyl-17-[3-(naphthalen-1-yloxy)propyl]-7,8,9,14-tetrahydro-5H-indolo[1,7-bc]pyrazolo-[4,3-e][2,8]benzodiazacycloundecine-16-carboxylic acid trifluoroacetate salt (enantiomer 2),
(rac)-4,5,8-trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid,
4,5,8-trimethyl-17-[3-(naphthalen-1-yloxy)propyl]-7,8,9,14-tetrahydro-5H-indolo[1,7-bc]-pyrazolo[4,3-e][2,8]benzodiazacycloundecine-16-carboxylic acid-N-ethylethanamine salt (enantiomer 1),
4,5,8-trimethyl-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid acetate salt,
4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid (enantiomer 1),
4,5-dimethyl-8-{[6-(morpholin-4-yl)pyridin-3-yl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-17-[3-(naphthalen-1-yloxy)propyl]-8-[(3,4,5-trimethoxybenzyl)carbamoyl]-7,8,9,14-tetrahydro-5H-indolo[1,7-bc]pyrazolo[4,3-e][2,8]benzodiazacycloundecine-16-carboxylic acid,
(rac)-4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic acid (enantiomer 1),
4,5-dimethyl-17-[3-(naphthalen-1-yloxy)propyl]-8-(tetrahydro-2H-pyran-4-ylacetyl)-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic acid (enantiomer 1),
4,5-dimethyl-17-[3-(naphthalen-1-yloxy)propyl]-8-(tetrahydro-2H-pyran-4-ylcarbamoyl)-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-17-[3-(naphthalen-1-yloxy)propyl]-8-[(tetrahydro-2H-pyran-4-ylmethyl)-carbamoyl]-7,8,9,14-tetrahydro-5H-indolo[1,7-bc]pyrazolo[4,3-e][2,8]benzodiazacycloundecine-16-carboxylic acid,
4,5-dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylic acid (enantiomer 1),
4,5-dimethyl-17-[3-(naphthalen-1-yloxy)propyl]-8-[(tetrahydro-2H-pyran-4-ylmethyl)carbamoyl]-7,8,9,14-tetrahydro-5H-indolo[1,7-bc]pyrazolo[4,3-e][2,8]benzodiazacycloundecine-16-carboxylic acid (enantiomer 2),
(rac)-4,5-dimethyl-8-{[2-(morpholin-4-yl)ethyl]sulfonyl}-17-[3-(naphthalen-1-yloxy)propyl]-7,8,9,14-tetrahydro-5H-indolo[1,7-bc]pyrazolo[4,3-e][2,8]benzodiazacycloundecine-16-carboxylic acid,
4,5-dimethyl-8-[2-(morpholin-4-yl)ethanesulfonyl]-17-{3-[(naphthalen-1-yl)oxy]propyl}-7,8,9,14-tetrahydro-5H-indolo[1,7-bc]pyrazolo[4,3-e][2,8]benzodiazacycloundecine-16-carboxylic acid-N-ethylethanamine salt (enantiomer 1),
4,5-dimethyl-8-[2-(morpholin-4-yl)ethanesulfonyl]-17-{3-[(naphthalen-1-yl)oxy]propyl}-7,8,9,14-tetrahydro-5H-indolo[1,7-bc]pyrazolo[4,3-e][2,8]benzodiazacycloundecine-16-carboxylic acid-N-ethylethanamine salt (enantiomer 2),
(rac)-3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid,
3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid-N-ethylethanamine salt (enantiomer 1),
3-chloro-4-ethyl-6-[2-(morpholin-4-yl)ethyl]-17-[3-(naphthalen-1-yloxy)propyl]-6,7,9,14-tetrahydroindolo[7,1-fg]pyrazolo[3,4-d][2,8]benzoxazacycloundecine-16-carboxylic acid-N-ethylethanamine salt (enantiomer 2),
(rac)-3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid,
3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid (enantiomer 1),
3-chloro-4,5-dimethyl-17-[3-(naphthalen-1-yloxy)propyl]-5,7,9,14-tetrahydroindolo[7,1-fg]pyrazolo[3,4-d][2,8]benzoxazacycloundecine-16-carboxylic acid (enantiomer 2),
(rac)-3-chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid,
3-chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid-N-ethylethanamine salt (enantiomer 1),
3-chloro-4,5-dimethyl-17-[3-(naphthalen-1-yloxy)propyl]-5,7,9,14-tetrahydropyrazolo-[4′,3′:9,10]pyrido[3′,2′:3,4][1,6]oxazacycloundecino[8,7,6-hi]indole-16-carboxylic acid-N-ethylethanamine salt (enantiomer 2),
(rac)-3-chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid,
3-chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid-N-ethylethanamine salt (enantiomer 1),
3-chloro-4,5-dimethyl-17-[3-(naphthalen-1-yloxy)propyl]-5,7,9,14-tetrahydropyrazolo-[4′,3′:9,10]pyrido[2′,3′:3,4][1,6]oxazacycloundecino[8,7,6-hi]indole-16-carboxylic acid-N-ethylethanamine salt (enantiomer 2),
(rac)-3-chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic acid,
3-chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic acid (enantiomer 1),
3-chloro-4,5-dimethyl-17-{3-[(naphthalen-1-yl)oxy]propyl}-5,7,9,14-tetrahydropyrazino-[2′,3′:3,4]pyrazolo[4′,3′:9,10][1,6]oxazacycloundecino[8,7,6-hi]indole-16-carboxylic acid (enantiomer 2),
(rac)-3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid,
3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 1),
3-chloro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic acid,
3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic acid (enantiomer 1),
3-chloro-4-ethyl-17-{3-[(6-fluoronaphthalen-1-yl)oxy]propyl}-6-[2-(morpholin-4-yl)ethyl]-6,7,9,14-tetrahydroindolo[7,1-fg]pyrazolo[3,4-d][2,8]benzoxazacycloundecine-16-carboxylic acid (enantiomer 2),
(rac)-3-chloro-4-ethyl-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic acid,
3-chloro-4-ethyl-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic acid N-ethylethanamine salt (enantiomer 1),
3-chloro-4-ethyl-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic acid N-ethylethanamine salt (enantiomer 2),
(rac)-3-chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid,
3-chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid-N-ethylethan-amine salt (enantiomer 1),
3-chloro-4-ethyl-17-{3-[(6-fluoronaphthalen-1-yl)oxy]propyl}-6-methyl-6,7,9,14-tetrahydro-indolo[7,1-fg]pyrazolo[3,4-d][2,8]benzoxazacycloundecine-16-carboxylic acid-N-ethylethanamine salt (enantiomer 2),
(rac)-1-(3-(4-chloro-3,5-dimethylphenoxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylic acid,
(rac)-1-(3-((2,3-dihydro-1H-inden-4-yl)oxy)propyl)-15-fluoro-13,14-dimethyl-4,9,11,13-tetrahydrobenzo[3,4]pyrazolo[4′,3′:9,10][1,6]oxaazacycloundecino[8,7,6-hi]indole-2-carboxylic acid,
(rac)-15-fluoro-13,14-dimethyl-1-(3-((5,6,7,8-tetrahydronaphthalen-1-yl)oxy)propyl)-4,9,11,13-tetrahydrobenzo[3,4]pyrazolo[4′,3′:9,10][1,6]oxaazacycloundecino[8,7,6-hi]indole-2-carboxylic acid,
(rac)-15-fluoro-1-(3-((4-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylic acid,
(rac)-15-fluoro-1-(3-((6-fluoronaphthalen-1-yl)oxy)propyl)-13,14-dimethyl-4,9,11,13-tetrahydrobenzo[3,4]pyrazolo[4′,3′:9,10][1,6]oxaazacycloundecino[8,7,6-hi]indole-2-carboxylic acid,
3-fluoro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 1),
3-fluoro-17-{3-[(6-fluoronaphthalen-1-yl)oxy]propyl}-4,5-dimethyl-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-15-fluoro-13,14-dimethyl-1-(3-(naphthalen-1-yloxy)propyl)-4,9,11,13-tetrahydrobenzo[3,4]pyrazolo[4′,3′:9,10][1,6]oxaazacycloundecino[8,7,6-hi]indole-2-carboxylic acid,
3-fluoro-4,5-dimethyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 1),
3-fluoro-4,5-dimethyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-14-ethyl-15-fluoro-13-methyl-1-(3-(naphthalen-1-yloxy)propyl)-4,9,11,13-tetrahydrobenzo[3,4]pyrazolo[4′,3′:9,10][1]oxa[6]azacycloundecino[8,7,6-hi]indole-2-carboxylic acid,
4-ethyl-3-fluoro-5-methyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 1),
4-ethyl-3-fluoro-5-methyl-17-{3-[(naphthalen-1-yl)oxy]propyl}-5,7,9,14-tetrahydroindolo[7,1-fg]pyrazolo[3,4-d][2,8]benzoxazacycloundecine-16-carboxylic acid-N-ethylethanamine salt (enantiomer 2),
(rac)-14-ethyl-15-fluoro-1-(3-((6-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylic acid,
4-ethyl-3-fluoro-17-{3-[(6-fluoronaphthalen-1-yl)oxy]propyl}-5-methyl-5,7,9,14-tetrahydroindolo[7,1-fg]pyrazolo[3,4-d][2,8]benzoxazacycloundecine-16-carboxylic acid-N-ethylethanamine salt (enantiomer 1),
4-ethyl-3-fluoro-17-{3-[(6-fluoronaphthalen-1-yl)oxy]propyl}-5-methyl-5,7,9,14-tetrahydro-indolo[7,1-fg]pyrazolo[3,4-d][2,8]benzoxazacycloundecine-16-carboxylic acid-N-ethylethan-amine salt (enantiomer 2),
(rac)-14-ethyl-15-fluoro-1-(3-((4-fluoronaphthalen-1-yl)oxy)propyl)-13-methyl-4,9,11,13-tetrahydrobenzo[3,4]pyrazolo[4′,3′:9,10][1]oxa[6]azacycloundecino[8,7,6-hi]indole-2-carboxylic acid,
(rac)-14-ethyl-15-fluoro-13-methyl-1-(3-((5,6,7,8-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylic acid,
(rac)-1-(3-((2,3-dihydro-1H-inden-4-yl)oxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylic acid,
(rac)-1-(3-(4-chloro-3,5-dimethylphenoxy)propyl)-14-ethyl-15-fluoro-13-methyl-4,9,11,13-tetrahydrobenzo[3,4]pyrazolo[4′,3′:9,10][1]oxa[6]azacycloundecino[8,7,6-hi]indole-2-carboxylic acid,
(rac)-15-fluoro-12,14-dimethyl-1-(3-(naphthalen-1-yloxy)propyl)-4,9,11,12-tetrahydrobenzo-[3,4]pyrazolo[4′,3′:9,10][1]oxa[6]azacycloundecino[8,7,6-hi]indole-2-carboxylic acid,
(rac)-15-fluoro-1-(3-((6-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-15-fluoro-1-(3-((4-fluoronaphthalen-1-yl)oxy)propyl)-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-15-fluoro-12,14-dimethyl-1-(3-((5,6,7,8-tetrahydronaphthalen-1-yl)oxy)propyl)-4,9,11,12-tetrahydrobenzo[3,4]pyrazolo[4′,3′:9,10][1]oxa[6]azacycloundecino[8,7,6-hi]indole-2-carboxylic acid,
(rac)-1-(3-((2,3-dihydro-1H-inden-4-yl)oxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-1-(3-(4-chloro-3,5-dimethylphenoxy)propyl)-15-fluoro-12,14-dimethyl-4,9,11,12-tetrahydrobenzo[3,4]pyrazolo[4′,3′:9,10][1]oxa[6]azacycloundecino[8,7,6-hi]indole-2-carboxylic acid,
(rac)-1-(3-(4-chloro-3,5-dimethylphenoxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-1-(3-((2,3-dihydro-1H-inden-4-yl)oxy)propyl)-14-ethyl-15-fluoro-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-12-methyl-1-(3-((5,6,7,8-tetrahydronaphthalen-1-yl)oxy)propyl)-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-1-(3-((4-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-1-(3-((6-fluoronaphthalen-1-yl)oxy)propyl)-12-methyl-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-12-methyl-1-(3-(naphthalen-1-yloxy)propyl)-4,9,11,12-tetrahydrobenzo[3,4]pyrazolo[4′,3′:9,10][1,6]oxaazacycloundecino[8,7,6-hi]indole-2-carboxylic acid,
(rac)-14-ethyl-15-fluoro-13-methyl-1-(3-((1,2,3,4-tetrahydronaphthalen-1-yl)oxy)propyl)-4,9,11,13-tetrahydrobenzo[3,4]pyrazolo[4′,3′:9,10][1]oxa[6]azacycloundecino[8,7,6-hi]indole-2-carboxylic acid,
(rac)-3-chloro-17-{3-[(6-fluoronaphthalen-1-yl)oxy]propyl}-4,5-dimethyl-5,7,9,14-tetrahydropyrazino[2′,3′:3,4]pyrazolo[4′,3′:9,10][1,6]oxazacycloundecino[8,7,6-hi]indole-16-carboxylic acid and
(rac)-3-chloro-4,5-dimethyl-16-[3-(1-naphthyloxy)propyl]-5,7-dihydro-9H,13H-[1,2]oxazolo[3′,4′:3,4]pyrazolo[4′,3′:9,10][1,6]oxazacycloundecino[8,7,6-hi]indole-15-carboxylic acid,
or a tautomer, an N-oxide, or a salt thereof, or a salt of a tautomer, or a salt of an N-oxide, or a mixture of same.

8. A method of preparing a compound of general formula (I) according to any one of claims 1 to 7, said method comprising the step of reacting an intermediate compound of general formula (II): 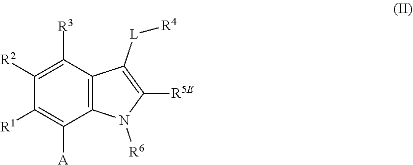
wherein R¹, R², R³, R⁴, R⁶, A and L are as defined for the compound of general formula (I) according to any one of claims 1 to 6, and R^{5E} represents a —C(═O)O—C₁₋₄-alkyl group or a benzyl ester group,
with an alkali hydroxide in a mixture of water and tetrahydrofuran and/or an aliphatic alcohol of formula C₁-C₃-alkyl-OH, at a temperature from 0° C. to 100° C.,
to transform the group R^{5E} into a group R⁵ as defined for the compounds of general formula (I), and subsequently optionally to convert the free acid group R⁵ into a pharmaceutically acceptable salt thereof to obtain a compound of general formula (I) 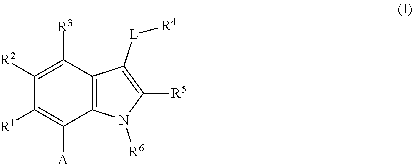
wherein R¹, R², R³, R⁴, R⁵, R⁶, A and L are as defined for the compound of general formula (I) according to any one of claims 1 to 6,
or a stereoisomer, a tautomer, an N-oxide, a hydrate, a solvate, or a salt thereof, or a mixture of same, and
optionally separating the enantiomers by means of preparative HPLC on a chiral stationary phase.

9. A compound of general formula (I) for use in a method of inhibiting proliferation of a cell and/or the induction of apoptosis in a cell, comprising contacting the cell with a compound of general formula (I) according to any one of claims 1-7.

10. A compound of general formula (I) according to any one of claims 1-7 for use in the treatment of diseases.

11. A compound for use according to claim 10, wherein the disease is a hyperproliferative disease.

12. A compound for use according to claim 11, wherein the hyperproliferative disease is cancer.

13. A compound for use according to claim 12, wherein the cancer is selected from breast cancer, lymphoma, leukemia, multiple myeloma, and ovarian cancer.

14. Use of a compound of claim 1 for the manufacture of a medicament for the treatment of a cancer selected from breast cancer, lymphoma, leukemia, multiple myeloma, and ovarian cancer.

15. A pharmaceutical composition comprising a compound of general formula (I) according to any one of claims 1 to 7 and one or more pharmaceutically acceptable excipients.

16. A pharmaceutical composition according to claim 15 for use according to claim 14.

17. A pharmaceutical composition according to claim 15 for use according to any one of claims 9-13.

18. A pharmaceutical combination comprising:
one or more compounds of general formula (I) according to any one of claims 1 to 7, and
one or more further anti-cancer agents.

19. An intermediate compound of general formula (II): 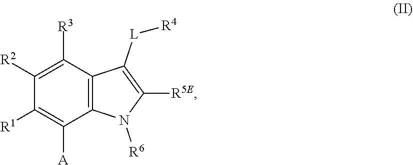
wherein R¹, R², R³, R⁴, R⁶, A and L are as defined for the compound of general formula (I) according to any one of claims 1 to 7, and R^{5E} represents a carboxylic ester group.

20. A method of using the intermediate compound of general formula (II) according to claim 19 for the preparation of a compound of general formula (I) according to any one of claims 1 to 7.
